(19) United States
(12) Patent Application Publication
(10) Pub. No.: US 2026/0226173 A1
(43) Pub. Date: Aug. 6, 2026

(54) COMPOSITIONS INCLUDING ANTI-WT-1 ANTIBODIES & ANTIGEN BINDING FRAGMENTS AND USES THEREOF

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US); Eureka Therapeutics, Inc., Emeryville, CA (US)

(72) Inventors: David A. SCHEINBERG, New York, NY (US); Tao DAO, New York, NY (US); Guangyan XIONG, Emeryville, CA (US); Jingyi XIANG, Emeryville, CA (US); Ziyou CUI, Emeryville, CA (US); Cheng LIU, Emeryville, CA (US)

(21) Appl. No.: 19/153,961

(22) PCT Filed: Feb. 5, 2024
(86) PCT No.: PCT/US2024/014515

§ 371 Date: Aug. 5, 2025

Related U.S. Application Data
(60) Provisional application No. 63/483,468, filed on Feb. 6, 2023.

(51) Int. Cl. C07K 16/28; A61K 35/17; A61K 38/00; A61K 40/11; C07K 14/705; C12N 15/62; G01N 33/53

(52) U.S. Cl. CPC C07K 16/2833; A61K 35/17; A61K 38/00; A61K 40/11; C07K 14/7051; C12N 15/625; G01N 33/53; A61K 2239/48; C07K 2317/21; C07K 2317/24; C07K 2317/31; C07K 2317/52; C07K 2317/565; C07K 2319/02; C07K 2319/03

(57) ABSTRACT

The present technology relates generally to compositions that specifically recognize and bind to a WT-1 peptide complexed with a major histocompatibility antigen (e.g., HL A-A*02). The compositions of the present technology are useful in methods for treating WT-1-associated diseases (e.g., cancers) in a subject in need thereof.

### CROSS-REFERENCE TO RELATED APPLICATIONS

This application is a 371 U.S. national phase application of International Application No. PCT/US2024/014515, filed Feb. 5, 2024, which claims the benefit of and priority to U.S. Provisional Patent Application No. 63/483,468, filed Feb. 6, 2023, the contents of both of which are incorporated herein by reference in their entirety.

### STATEMENT OF GOVERNMENT SUPPORT

This invention was made with government support under CA55349, CA23766, CA241894 and CA265328-01 awarded by the National Institutes of Health. The government has certain rights in the invention.

### SEQUENCE LISTING

The instant application contains a Sequence Listing which has been submitted in XML format and is hereby incorporated by reference in its entirety. Said XML copy, created on Apr. 9, 2024, is named 115872-2818_SL.xml and is 306,947 bytes.

### TECHNICAL FIELD

The present disclosure provides compositions that specifically bind to WT1-peptide complexed with MHC, including antibodies such as human, humanized, or chimeric antibodies, antibody fragments, chimeric antibody-T cell receptors (caTCRs), chimeric antigen receptors (CARs), chimeric signaling receptors (CSRs), fusion proteins, and conjugates thereof. The compositions of the present technology bind to HLA-A*02-restricted WT1 peptides and are useful for the treatment of WT1-associated diseases, including but not limited to cancers.

### BACKGROUND

The following description of the background of the present technology is provided simply as an aid in understanding the present technology and is not admitted to describe or constitute prior art to the present technology.

Chimeric antigen receptor (CAR) T cell therapy has achieved remarkable clinical efficacy in B cell hematological malignancies (1, 2). However, CAR T cells also elicit toxicities including cytokine release syndrome (CRS), neurologic toxicity and on target/off tumor recognition (3). Current CAR constructs include an antigen-binding domain, usually a single-chain variable fragment (scFv) derived from a monoclonal antibody that recognizes antigen on tumor cells, which is linked to an intracellular signaling domain derived from CD3ζ, a component of the T cell receptor (TCR) complex, as well as a costimulatory domain that often comprises a region of at least one costimulatory molecule such as CD28 or 4-1BB. Direct fusion of the antigen recognition domain to the downstream T cell activation domain from CD3ζ, however, can create an exaggerated synthetic activation signal that results in excessive T cell activation and cytokine release as well as exhaustion of T cells (4-6).

Additional challenges limit the traditional class of CAR T cell agents for the treatment of cancer types other than B cell cancers. Most important is the paucity of tumor-specific antigens for solid tumors and other hematological malignancies, such AML, that may be addressed without significant risk of life-threatening normal cell killing (8, 9). Identification of ideal target antigens that are ubiquitously expressed on all tumor cells but are absent or only minimally expressed on normal tissues is a major challenge. Most currently used targets of CAR-T therapy may result in “on-target/off-tumor” toxicity. In AML, myeloid lineage antigens such as CD33, CD123 and many others are shared between leukemia blasts and normal myeloid cells, which could lead to severe myelosuppression after effective CAR T cell therapy. In contrast, intracellular tumor antigens that may be more tumor-specific have been infrequently explored in CAR-T cell therapy (10). The generation and therapeutic efficacy in models of a human TCR mimic mAb (TCRm), ESK1, specific for the Wilm's tumor protein (WT1)-derived RMFPNAPYL (RMF) (SEQ ID NO: 113) peptide in the context of HLA-A0201 molecules has been previously described (11, 12). WT1 oncoprotein is an intracellular, oncogenic transcription factor that is selectively overexpressed in a wide range of leukemias (13) and solid cancers (14). RMFPNAPYL (RMF) (SEQ ID NO: 113), is a well-validated epitope of WT1 that has been explored preclinically and clinically, including peptide or dendritic cell vaccinations, and adoptive T cell therapy and in CAR T cell and bispecific antibody formats (2, 15-19). The original RMF/HLA-A2-specific TCRm ESK1 had liabilities related to off-target reactivity (20, 21).

Accordingly, there is an urgent need for anti-WT1 peptide/MHC TCRm antibody agents that exhibit improved target specificity.

### SUMMARY OF THE PRESENT TECHNOLOGY

The present application in one aspect provides constructs (such as isolated constructs) that bind to a complex comprising a WT1 peptide and an MHC class I protein (referred to herein as a “WT1/MHC class I complex,” or “WTMC”). In some embodiments, the constructs (“anti-WTMC constructs”) comprise an antibody moiety (referred to herein as an “anti-WTMC antibody moiety”) that specifically binds to a complex comprising a WT1 peptide and an MHC class I protein.

In some embodiments, there is provided an anti-WTMC (WT1/major histocompatibility class I protein complex) construct comprising an antibody moiety that specifically binds to a complex comprising WT1 peptide (e.g., WT1-RMF) and a major histocompatibility (MHC) class I protein, wherein the WT1 comprises the amino acid sequence of SEQ ID NO: 113. In some embodiments of the anti-WTMC construct, the antibody moiety specifically recognizes amino acid residues FPNA (SEQ ID NO: 349) of SEQ ID NO: 113. In some embodiments, the MHC class I protein is HLA-A*02:01.

In some embodiments according to the anti-WTMC described above, the antibody moiety comprises: a V_{H} comprising a HC-CDR1 comprising the amino acid sequence of any one of SEQ ID NOs: 1, 7, 13, 19, 25, 31, 37, 43, 49, 55, 61, 67, 73, or 79, or a variant thereof comprising up to about 3 amino acid substitutions, an HC-CDR2 comprising the amino acid sequence of any one of SEQ ID NOs: 2, 8, 14, 20, 26, 32, 38, 44, 50, 56, 62, 68, 74, or 80, or a variant thereof comprising up to about 3 amino acid substitutions, and an HC-CDR3 comprising the amino acid sequence of any one of SEQ ID NOs: 3, 9, 15, 21, 27, 33, 39, 45, 51, 57, 63, 69, 75 or 81, or a variant thereof comprising up to about 3 amino acid substitutions; and a V_{L} comprising a LC-CDR1 comprising the amino acid sequence of any one of SEQ ID NOs: 4, 10, 16, 22, 28, 34, 40, 46, 52, 58, 64, 70, 76, or 82, or a variant thereof comprising up to about 3 amino acid substitutions, an LC-CDR2 comprising the amino acid sequence of any one of SEQ ID NOs: 5, 11, 17, 23, 29, 35, 41, 47, 53, 59, 65, 71, 77, or 83, or a variant thereof comprising about 2 amino acid substitutions, and an LC-CDR3 comprising the amino acid sequence of any one of SEQ ID NOs: 6, 12, 18, 24, 30, 36, 42, 48, 54, 60, 66, 72, 78 or 84, or a variant thereof comprising up to about 3 amino acid substitutions.

In some embodiments, the antibody moiety comprises: (i) a V_{H} comprising an HC-CDR1 comprising the amino acid sequence of any one of SEQ ID NOs: 1, 7, 13, 19, 25, 31, 37, 43, 49, 55, 61, 67, 73, or 79, an HC-CDR2 comprising the amino acid sequence of any one of SEQ ID NOs: 2, 8, 14, 20, 26, 32, 38, 44, 50, 56, 62, 68, 74, or 80, and an HC-CDR3 comprising the amino acid sequence of any one of SEQ ID NOs: 3, 9, 15, 21, 27, 33, 39, 45, 51, 57, 63, 69, 75 or 81; and (ii) a V_{L} comprising an LC-CDR1 comprising the amino acid sequence of any one of SEQ ID NOs: 4, 10, 16, 22, 28, 34, 40, 46, 52, 58, 64, 70, 76, or 82, an LC-CDR2 comprising the amino acid sequence of any one of SEQ ID NOs: 5, 11, 17, 23, 29, 35, 41, 47, 53, 59, 65, 71, 77, or 83, and an LC-CDR3 comprising the amino acid sequence of any one of SEQ ID NOs: 6, 12, 18, 24, 30, 36, 42, 48, 54, 60, 66, 72, 78 or 84.

In some embodiments, there is provided an anti-WTMC construct comprising an antibody moiety that comprises: (i) a heavy chain immunoglobulin variable domain (V_{H}) comprising a heavy chain complementarity determining region (HC-CDR) 1 sequence, a HC-CDR2 sequence, and a HC-CDR3 sequence of the V_{H} sequence of SEQ ID NO: 85, and a light chain immunoglobulin variable domain (V_{L}) comprising a light chain complementarity determining region (LC-CDR) 1 sequence, a LC-CDR2 sequence, and a LC-CDR3 sequence of the V_{L} sequence SEQ ID NO: 86; or (ii) a V_{H} comprising a HC-CDR1 sequence, a HC-CDR2 sequence, and a HC-CDR3 sequence of the V_{H} sequence of SEQ ID NO: 87, and a V_{L} comprising a LC-CDR1 sequence, a LC-CDR2 sequence, and a LC-CDR3 sequence of the V_{L} sequence SEQ ID NO: 88; or (iii) a V_{H} comprising aHC-CDR1 sequence, a HC-CDR2 sequence, and a HC-CDR3 sequence of the V_{H} sequence of SEQ ID NO: 89, and a V_{L} comprising a LC-CDR1 sequence, a LC-CDR2 sequence, and a LC-CDR3 sequence of the V_{L} sequence SEQ ID NO: 90; or (iv) a V_{H} comprising a HC-CDR1 sequence, a HC-CDR2 sequence, and a HC-CDR3 sequence of the V_{H} sequence of SEQ ID NO: 91, and a V_{L} comprising a LC-CDR1 sequence, a LC-CDR2 sequence, and a LC-CDR3 sequence of the V_{L} sequence SEQ ID NO: 92; or (v) a V_{H} comprising a HC-CDR1 sequence, a HC-CDR2 sequence, and a HC-CDR3 sequence of the V_{H} sequence of SEQ ID NO: 93, and a V_{L} comprising a LC-CDR1 sequence, a LC-CDR2 sequence, and a LC-CDR3 sequence of the V_{L} sequence SEQ ID NO: 94; or (vi) a V_{H} comprising a HC-CDR1 sequence, a HC-CDR2 sequence, and a HC-CDR3 sequence of the V_{H} sequence of SEQ ID NO: 95, and a V_{L} comprising a LC-CDR1 sequence, a LC-CDR2 sequence, and a LC-CDR3 sequence of the V_{L} sequence SEQ ID NO: 96; or (vii) a V_{H} comprising a HC-CDR1 sequence, a HC-CDR2 sequence, and a HC-CDR3 sequence of the V_{H} sequence of SEQ ID NO: 97, and a V_{L} comprising a LC-CDR1 sequence, a LC-CDR2 sequence, and a LC-CDR3 sequence of the V_{L} sequence SEQ ID NO: 98; or (viii) a V_{H} comprising a HC-CDR1 sequence, a HC-CDR2 sequence, and a HC-CDR3 sequence of the V_{H} sequence of SEQ ID NO: 99, and a V_{L} comprising a LC-CDR1 sequence, a LC-CDR2 sequence, and a LC-CDR3 sequence of the V_{L} sequence SEQ ID NO: 100; or (ix) a V_{H} comprising a HC-CDR1 sequence, a HC-CDR2 sequence, and a HC-CDR3 sequence of the V_{H} sequence of SEQ ID NO: 101, and a V_{L} comprising a LC-CDR1 sequence, a LC-CDR2 sequence, and a LC-CDR3 sequence of the V_{L} sequence SEQ ID NO: 102; or (x) a V_{H} comprising a HC-CDR1 sequence, a HC-CDR2 sequence, and a HC-CDR3 sequence of the V_{H} sequence of SEQ ID NO: 103, and a V_{L} comprising a LC-CDR1 sequence, a LC-CDR2 sequence, and a LC-CDR3 sequence of the V_{L} sequence SEQ ID NO: 104; or (xi) a V_{H} comprising a HC-CDR1 sequence, a HC-CDR2 sequence, and a HC-CDR3 sequence of the V_{H} sequence of SEQ ID NO: 105, and a V_{L} comprising a LC-CDR1 sequence, a LC-CDR2 sequence, and a LC-CDR3 sequence of the V_{L} sequence SEQ ID NO: 106; or (xii) a V_{H} comprising a HC-CDR1 sequence, a HC-CDR2 sequence, and a HC-CDR3 sequence of the V_{H} sequence of SEQ ID NO: 107, and a V_{L} comprising a LC-CDR1 sequence, a LC-CDR2 sequence, and a LC-CDR3 sequence of the V_{L} sequence SEQ ID NO: 108; or (xiii) a V_{H} comprising a HC-CDR1 sequence, a HC-CDR2 sequence, and a HC-CDR3 sequence of the V_{H} sequence of SEQ ID NO: 109, and a V_{L} comprising a LC-CDR1 sequence, a LC-CDR2 sequence, and a LC-CDR3 sequence of the V_{L} sequence SEQ ID NO: 110; or (xiv) a V_{H} comprising a HC-CDR1 sequence, a HC-CDR2 sequence, and a HC-CDR3 sequence of the V_{H} sequence of SEQ ID NO: 111, and a V_{L} comprising a LC-CDR1 sequence, a LC-CDR2 sequence, and a LC-CDR3 sequence of the V_{L} sequence SEQ ID NO: 112.

In some embodiments, the construct specifically binds to a complex comprising WT1 and an MHC class I protein, wherein the WT1-RMF comprises the amino acid sequence of SEQ ID NO: 113. In some embodiments of the anti-WTMC construct, the antibody moiety specifically recognizes amino acid residues FPNA (SEQ ID NO: 349) of SEQ ID NO: 113. In some embodiments, the MHC class I protein is HLA-A*02:01.

In some embodiments according to any of the anti-WTMC constructs described above, the antibody moiety comprises: (a) i) a V_{H} comprising an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 3; and ii) a V_{L} comprising an LC-CDR1 comprising the amino acid sequence of SEQ ID NO: 4, an LC-CDR2 comprising the amino acid sequence of SEQ ID NO: 5, and an LC-CDR3 comprising the amino acid sequence of SEQ ID NO: 6; (b) i) a V_{H} comprising an HC-CDR1 comprising the amino acid sequence of SEQ ID NO: 7, an HC-CDR2 comprising the amino acid sequence of SEQ ID NO: 8, and an HC-CDR3 comprising the amino acid sequence of SEQ ID NO: 9; and ii) a V_{L} comprising an LC-CDR1 comprising the amino acid sequence of SEQ ID NO: 10, an LC-CDR2 comprising the amino acid sequence of SEQ ID NO: 11, and an LC-CDR3 comprising the amino acid sequence of SEQ ID NO: 12; (c) i) a V_{H} comprising an HC-CDR1 comprising the amino acid sequence of SEQ ID NO: 13, an HC-CDR2 comprising the amino acid sequence of SEQ ID NO: 14, and an HC-CDR3 comprising the amino acid sequence of SEQ ID NO: 15; and ii) a light chain variable domain (V_{L}) comprising an LC-CDR1 comprising the amino acid sequence of SEQ ID NO: 16, an LC-CDR2 comprising the amino acid sequence of SEQ ID NO: 17, and an LC-CDR3 comprising the amino acid sequence of SEQ ID NO: 18; (d) i) a V_{H} comprising an HC-CDR1 comprising the amino acid sequence of SEQ ID NO: 19, an HC-CDR2 comprising the amino acid sequence of SEQ ID NO: 20, and an HC-CDR3 comprising the amino acid sequence of SEQ ID NO: 21; and ii) a light chain variable domain (V_{L}) comprising an LC-CDR1 comprising the amino acid sequence of SEQ ID NO: 22, an LC-CDR2 comprising the amino acid sequence of SEQ ID NO: 23, and an LC-CDR3 comprising the amino acid sequence of SEQ ID NO: 24; or (e) i) a V_{H} comprising an HC-CDR1 comprising the amino acid sequence of SEQ ID NO: 25, an HC-CDR2 comprising the amino acid sequence of SEQ ID NO: 26, and an HC-CDR3 comprising the amino acid sequence of SEQ ID NO: 27; and ii) a light chain variable domain (V_{L}) comprising an LC-CDR1 comprising the amino acid sequence of SEQ ID NO: 28, an LC-CDR2 comprising the amino acid sequence of SEQ ID NO: 29, and an LC-CDR3 comprising the amino acid sequence of SEQ ID NO: 30; or (f) i) a V_{H} comprising an HC-CDR1 comprising the amino acid sequence of SEQ ID NO: 31, an HC-CDR2 comprising the amino acid sequence of SEQ ID NO: 32, and an HC-CDR3 comprising the amino acid sequence of SEQ ID NO: 33; and ii) a light chain variable domain (V_{L}) comprising an LC-CDR1 comprising the amino acid sequence of SEQ ID NO: 34, an LC-CDR2 comprising the amino acid sequence of SEQ ID NO: 35, and an LC-CDR3 comprising the amino acid sequence of SEQ ID NO: 36; or (g) i) a V_{H} comprising an HC-CDR1 comprising the amino acid sequence of SEQ ID NO: 37, an HC-CDR2 comprising the amino acid sequence of SEQ ID NO: 38, and an HC-CDR3 comprising the amino acid sequence of SEQ ID NO: 39; and ii) a light chain variable domain (V_{L}) comprising an LC-CDR1 comprising the amino acid sequence of SEQ ID NO: 40, an LC-CDR2 comprising the amino acid sequence of SEQ ID NO: 41, and an LC-CDR3 comprising the amino acid sequence of SEQ ID NO: 42; or (h) i) a V_{H} comprising an HC-CDR1 comprising the amino acid sequence of SEQ ID NO: 43, an HC-CDR2 comprising the amino acid sequence of SEQ ID NO: 44, and an HC-CDR3 comprising the amino acid sequence of SEQ ID NO: 45; and ii) a light chain variable domain (V_{L}) comprising an LC-CDR1 comprising the amino acid sequence of SEQ ID NO: 46, an LC-CDR2 comprising the amino acid sequence of SEQ ID NO: 47, and an LC-CDR3 comprising the amino acid sequence of SEQ ID NO: 48; or (i) i) a V_{H} comprising an HC-CDR1 comprising the amino acid sequence of SEQ ID NO: 49, an HC-CDR2 comprising the amino acid sequence of SEQ ID NO: 50, and an HC-CDR3 comprising the amino acid sequence of SEQ ID NO: 51; and ii) a light chain variable domain (V_{L}) comprising an LC-CDR1 comprising the amino acid sequence of SEQ ID NO: 52, an LC-CDR2 comprising the amino acid sequence of SEQ ID NO: 53, and an LC-CDR3 comprising the amino acid sequence of SEQ ID NO: 54; or (j) i) a V_{H} comprising an HC-CDR1 comprising the amino acid sequence of SEQ ID NO: 55, an HC-CDR2 comprising the amino acid sequence of SEQ ID NO: 56, and an HC-CDR3 comprising the amino acid sequence of SEQ ID NO: 57; and ii) a light chain variable domain (V_{L}) comprising an LC-CDR1 comprising the amino acid sequence of SEQ ID NO: 58, an LC-CDR2 comprising the amino acid sequence of SEQ ID NO: 59, and an LC-CDR3 comprising the amino acid sequence of SEQ ID NO: 60; or (k) i) a V_{H} comprising an HC-CDR1 comprising the amino acid sequence of SEQ ID NO: 61, an HC-CDR2 comprising the amino acid sequence of SEQ ID NO: 62, and an HC-CDR3 comprising the amino acid sequence of SEQ ID NO: 63; and ii) a light chain variable domain (V_{L}) comprising an LC-CDR1 comprising the amino acid sequence of SEQ ID NO: 64, an LC-CDR2 comprising the amino acid sequence of SEQ ID NO: 65, and an LC-CDR3 comprising the amino acid sequence of SEQ ID NO: 66; or (1) i) a V_{H} comprising an HC-CDR1 comprising the amino acid sequence of SEQ ID NO: 67, an HC-CDR2 comprising the amino acid sequence of SEQ ID NO: 68, and an HC-CDR3 comprising the amino acid sequence of SEQ ID NO: 69; and ii) a light chain variable domain (V_{L}) comprising an LC-CDR1 comprising the amino acid sequence of SEQ ID NO: 70, an LC-CDR2 comprising the amino acid sequence of SEQ ID NO: 71, and an LC-CDR3 comprising the amino acid sequence of SEQ ID NO: 72; or (m) i) a V_{H} comprising an HC-CDR1 comprising the amino acid sequence of SEQ ID NO: 73, an HC-CDR2 comprising the amino acid sequence of SEQ ID NO: 74, and an HC-CDR3 comprising the amino acid sequence of SEQ ID NO: 75; and ii) a light chain variable domain (V_{L}) comprising an LC-CDR1 comprising the amino acid sequence of SEQ ID NO: 76, an LC-CDR2 comprising the amino acid sequence of SEQ ID NO: 77, and an LC-CDR3 comprising the amino acid sequence of SEQ ID NO: 78; or (n) i) a V_{H} comprising an HC-CDR1 comprising the amino acid sequence of SEQ ID NO: 79, an HC-CDR2 comprising the amino acid sequence of SEQ ID NO: 80, and an HC-CDR3 comprising the amino acid sequence of SEQ ID NO: 81; and ii) a light chain variable domain (V_{L}) comprising an LC-CDR1 comprising the amino acid sequence of SEQ ID NO: 82, an LC-CDR2 comprising the amino acid sequence of SEQ ID NO: 83, and an LC-CDR3 comprising the amino acid sequence of SEQ ID NO: 84.

In some embodiments according to any of the anti-WTMC constructs described above, the antibody moiety comprises: (1) i) a V_{H} comprising the amino acid sequence of SEQ ID NO: 85, or a variant thereof having at least about 95% sequence identity to the amino acid sequence of SEQ ID NO: 85; and ii) a V_{L} comprising the amino acid sequence of SEQ ID NO: 86, or a variant thereof having at least about 95% sequence identity to the amino acid sequence of SEQ ID NO: 86; or (2) i) a V_{H} comprising the amino acid sequence of SEQ ID NO: 87, or a variant thereof having at least about 95% sequence identity to the amino acid sequence of SEQ ID NO: 87; and ii) a V_{L} comprising the amino acid sequence of SEQ ID NO: 88, or a variant thereof having at least about 95% sequence identity to the amino acid sequence of SEQ ID NO: 88; or (3) i) a V_{H} comprising the amino acid sequence of SEQ ID NO: 89, or a variant thereof having at least about 95% sequence identity to the amino acid sequence of SEQ ID NO: 89; and ii) a V_{L} comprising the amino acid sequence of SEQ ID NO: 90, or a variant thereof having at least about 95% sequence identity to the amino acid sequence of SEQ ID NO: 90; or (4) i) a V_{H} comprising the amino acid sequence of SEQ ID NO: 91, or a variant thereof having at least about 95% sequence identity to the amino acid sequence of SEQ ID NO: 91; and ii) a V_{L} comprising the amino acid sequence of SEQ ID NO: 92, or a variant thereof having at least about 95% sequence identity to the amino acid sequence of SEQ ID NO: 92; or (5) i) a V_{H} comprising the amino acid sequence of SEQ ID NO: 93, or a variant thereof having at least about 95% sequence identity to the amino acid sequence of SEQ ID NO: 93; and ii) a V_{L} comprising the amino acid sequence of SEQ ID NO: 94, or a variant thereof having at least about 95% sequence identity to the amino acid sequence of SEQ ID NO: 94; or (6) i) a V_{H} comprising the amino acid sequence of SEQ ID NO: 95, or a variant thereof having at least about 95% sequence identity to the amino acid sequence of SEQ ID NO: 95; and ii) a V_{L} comprising the amino acid sequence of SEQ ID NO: 96, or a variant thereof having at least about 95% sequence identity to the amino acid sequence of SEQ ID NO: 96; or (7) i) a V_{H} comprising the amino acid sequence of SEQ ID NO: 97, or a variant thereof having at least about 95% sequence identity to the amino acid sequence of SEQ ID NO: 97; and ii) a V_{L} comprising the amino acid sequence of SEQ ID NO: 98, or a variant thereof having at least about 95% sequence identity to the amino acid sequence of SEQ ID NO: 98; or (8) i) a V_{H} comprising the amino acid sequence of SEQ ID NO: 99, or a variant thereof having at least about 95% sequence identity to the amino acid sequence of SEQ ID NO: 99; and ii) a V_{L} comprising the amino acid sequence of SEQ ID NO: 100, or a variant thereof having at least about 95% sequence identity to the amino acid sequence of SEQ ID NO: 100; or (9) i) a V_{H} comprising the amino acid sequence of SEQ ID NO: 101, or a variant thereof having at least about 95% sequence identity to the amino acid sequence of SEQ ID NO: 101; and ii) a V_{L} comprising the amino acid sequence of SEQ ID NO: 102, or a variant thereof having at least about 95% sequence identity to the amino acid sequence of SEQ ID NO: 102; or (10) i) a V_{H} comprising the amino acid sequence of SEQ ID NO: 103, or a variant thereof having at least about 95% sequence identity to the amino acid sequence of SEQ ID NO: 103; and ii) a V_{L} comprising the amino acid sequence of SEQ ID NO: 104, or a variant thereof having at least about 95% sequence identity to the amino acid sequence of SEQ ID NO: 104; or (11) i) a V_{H} comprising the amino acid sequence of SEQ ID NO: 105, or a variant thereof having at least about 95% sequence identity to the amino acid sequence of SEQ ID NO: 105; and ii) a V_{L} comprising the amino acid sequence of SEQ ID NO: 106, or a variant thereof having at least about 95% sequence identity to the amino acid sequence of SEQ ID NO: 106; or (12) i) a V_{H} comprising the amino acid sequence of SEQ ID NO: 107, or a variant thereof having at least about 95% sequence identity to the amino acid sequence of SEQ ID NO: 107; and ii) a V_{L} comprising the amino acid sequence of SEQ ID NO: 108, or a variant thereof having at least about 95% sequence identity to the amino acid sequence of SEQ ID NO: 108; or (13) i) a V_{H} comprising the amino acid sequence of SEQ ID NO: 109, or a variant thereof having at least about 95% sequence identity to the amino acid sequence of SEQ ID NO: 109; and ii) a V_{L} comprising the amino acid sequence of SEQ ID NO: 110, or a variant thereof having at least about 95% sequence identity to the amino acid sequence of SEQ ID NO: 110; or (14) i) a V_{H} comprising the amino acid sequence of SEQ ID NO: 111, or a variant thereof having at least about 95% sequence identity to the amino acid sequence of SEQ ID NO: 111; and ii) a V_{L} comprising the amino acid sequence of SEQ ID NO: 112, or a variant thereof having at least about 95% sequence identity to the amino acid sequence of SEQ ID NO: 112.

In some embodiments according to any of the anti-WTMC constructs described above, the antibody moiety comprises (1) i) a V_{H} comprising the amino acid sequence of SEQ ID NO: 85; and ii) a V_{L} comprising the amino acid sequence of SEQ ID NO: 86; or (2) i) a V_{H} comprising the amino acid sequence of SEQ ID NO: 87; and ii) a V_{L} comprising the amino acid sequence of SEQ ID NO: 88; or (3) i) a V_{H} comprising the amino acid sequence of SEQ ID NO: 89; and ii) a V_{L} comprising the amino acid sequence of SEQ ID NO: 90; or (4) i) a V_{H} comprising the amino acid sequence of SEQ ID NO: 91; and ii) a V_{L} comprising the amino acid sequence of SEQ ID NO: 92; or (5) i) a V_{H} comprising the amino acid sequence of SEQ ID NO: 93; and ii) a V_{L} comprising the amino acid sequence of SEQ ID NO: 94; or (6) i) a V_{H} comprising the amino acid sequence of SEQ ID NO: 95; and ii) a V_{L} comprising the amino acid sequence of SEQ ID NO: 96; or (7) i) a V_{H} comprising the amino acid sequence of SEQ ID NO: 97; and ii) a V_{L} comprising the amino acid sequence of SEQ ID NO: 98; or (8) i) a V_{H} comprising the amino acid sequence of SEQ ID NO: 99; and ii) a V_{L} comprising the amino acid sequence of SEQ ID NO: 100; or (9) i) a V_{H} comprising the amino acid sequence of SEQ ID NO: 101; and ii) a V_{L} comprising the amino acid sequence of SEQ ID NO: 102; or (10) i) a V_{H} comprising the amino acid sequence of SEQ ID NO: 103; and ii) a V_{L} comprising the amino acid sequence of SEQ ID NO: 104; or (11) i) a V_{H} comprising the amino acid sequence of SEQ ID NO: 105; and ii) a V_{L} comprising the amino acid sequence of SEQ ID NO: 106; or (12) i) a V_{H} comprising the amino acid sequence of SEQ ID NO: 107; and ii) a V_{L} comprising the amino acid sequence of SEQ ID NO: 108; or (13) i) a V_{H} comprising the amino acid sequence of SEQ ID NO: 109; and ii) a V_{L} comprising the amino acid sequence of SEQ ID NO: 110; or (14) i) a V_{H} comprising the amino acid sequence of SEQ ID NO: 111; and ii) a V_{L} comprising the amino acid sequence of SEQ ID NO: 112.

In some embodiments according to any of the anti-WTMC constructs described above, the antibody moiety is chimeric, humanized, partially human, fully human, or semi-synthetic. In some embodiments, the antibody moiety is a full-length antibody, a Fab, a Fab′, a F(ab′)₂, an Fv, or a single chain Fv (scFv). In some embodiments, the antibody moiety is an scFv. In some embodiments, the scFv comprises the amino acid sequence of any one of SEQ ID NOs: 335-348. In some embodiments, the antibody moiety is a Fab or Fab′. In some embodiments, the antibody moiety specifically recognizing WTMC is fused to an Fc fragment, optionally via a linker. In some embodiments, the Fc fragment is an IgG Fc fragment. In some embodiments, the IgG is an IgG1, IgG2, IgG3, or IgG4. In some embodiments, the anti-WTMC construct is a full-length antibody. In some embodiments, the anti-WTMC construct is monospecific.

In some embodiments according to any of the anti-WTMC constructs described above, the anti-WTMC construct is multispecific. In some embodiments, the anti-WTMC construct is bispecific. In some embodiments, the anti-WTMC construct is a tandem scFv, a diabody (Db), a single chain diabody (scDb), a dual-affinity retargeting (DART) antibody, a F(ab′)2, a dual variable domain (DVD) antibody, a knob-into-hole (KiH) antibody, a dock and lock (DNL) antibody, a chemically cross-linked antibody, a heteromultimeric antibody, or a heteroconjugate antibody. In some embodiments, the anti-WTMC construct is a tandem scFv comprising two scFvs linked by a peptide linker. In some embodiments, the anti-WTMC construct further comprises a second antibody moiety specifically recognizing a second antigen. In some embodiments, the second antigen is an antigen on the surface of a T cell. In some embodiments, the second antigen is selected from the group consisting of CD3, CD3γ, CD3δ, CD3ε, CD3ζ, CD28, OX40, GITR, CD137, CD27, CD40L, and HVEM. In some embodiments, the second antigen is CD3ε. In some embodiments, the anti-WTMC construct is a tandem scFv comprising an N-terminal scFv specifically recognizing WTMC and a C-terminal scFv specifically recognizing CD3ε. In some embodiments, the T cell is selected from the group consisting of a cytotoxic T cell, a helper T cell, and a natural killer T (NKT) cell. In some embodiments, the expression of the anti-WTMC construct is induced by the activation of an engineered T cell or natural killer (NK) cell. In some embodiments, the engineered T cell or NK cell is a T cell or NK cell comprising a chimeric antigen receptor (CAR). In some embodiments, the engineered T cell or NK cell is a T cell or NK cell comprising a chimeric antibody-T cell receptor construct (caTCR). In some embodiments, the second antigen is an antigen on the surface of a B cell, a natural killer cell, a dendritic cell, a macrophage, a monocyte, or a neutrophil.

In some embodiments according to any of the anti-WTMC constructs described above, the anti-WTMC construct is a CAR comprising: (a) an extracellular domain comprising the antibody moiety; (b) a transmembrane domain; and (c) an intracellular signaling domain. In some embodiments, the intracellular signaling domain comprises a primary immune cell signaling sequence derived from CD3ζ (i.e., TCRζ), FcRγ, FcRβ, CD3γ, CD3δ, CD3ε, CD5, CD22, CD79a, CD79b, or CD66d. In some embodiments, the intracellular signaling domain further comprise a costimulatory signaling sequence derived from CD28, CD30, 4-1BB, DAP10, ICOS, or OX40. In some embodiments, the intracellular signaling domain comprises a CD3ζ intracellular signaling sequence and a CD28 intracellular signaling sequence. In some embodiments, the intracellular signaling domain comprises a CD3ζ intracellular signaling sequence and a CD30 intracellular signaling sequence. In some embodiments, the intracellular signaling domain comprises a CD3ζ intracellular signaling sequence and a 4-1BB intracellular signaling sequence.

In some embodiments according to any of the anti-WTMC constructs described above, the anti-WTMC construct is a caTCR comprising: (a) an extracellular domain comprising the antibody moiety; and (b) a T cell receptor module (TCRM) comprising a first TCR domain (TCRD) comprising a first TCR transmembrane domain (TCR-TM) and a second TCRD comprising a second TCR-TM, wherein the TCRM is capable of associating with at least one TCR-associated signaling molecule. In some embodiments, the caTCR lacks a functional primary immune cell signaling domain of a TCR-associated T cell activation molecule selected from the group consisting of CD3ζ (TCRζ), FcRγ, FcRβ, CD3γ, CD3δ, CD3ε, CD5, CD22, CD79a, CD79b, and CD66d. Additionally or alternatively, in some embodiments, the caTCR lacks any primary immune cell signaling sequences. In some embodiments, the first TCR-TM is derived from one of the transmembrane domains of a first naturally occurring TCR and the second TCR-TM is derived from the other transmembrane domain of the first naturally occurring TCR. In some embodiments, at least one of the TCR-TMs is non-naturally occurring. In some embodiments, the TCRM comprising the at least one non-naturally occurring TCR-TM allows for enhanced association of the at least one TCR-associated signaling molecule as compared to a TCRM comprising the first naturally occurring T cell receptor transmembrane domains. In some embodiments, the first TCR-TM and the second TCR-TM are derived from a γ/δ TCR. In some embodiments, the first TCR-TM and the second TCR-TM are derived from an α/β TCR. In some embodiments, the TCR-associated signaling molecule is selected from the group consisting of CD3δε, CD3γε, and ζζ.

In some embodiments according to any of the anti-WTMC constructs described above, the construct is a chimeric signaling receptor (CSR) comprising: i) a target-binding module comprising the antibody moiety; ii) a transmembrane module; and iii) a co-stimulatory immune cell signaling module that is capable of providing a co-stimulatory signal to the effector cell, and wherein the CSR lacks a functional primary immune cell signaling domain of a TCR-associated T cell activation molecule selected from the group consisting of CD3ζ (TCRζ), FcRγ, FcRβ, CD3γ, CD3δ, CD3ε, CD5, CD22, CD79a, CD79b, and CD66d. In some embodiments, the CSR lacks any primary immune cell signaling sequences. In some embodiments, the transmembrane module of the CSR and the co-stimulatory immune cell signaling module of the CSR are from the same molecule. In some embodiments, the transmembrane module of the CSR and the co-stimulatory immune cell signaling module of the CSR are from different molecules. In some embodiments, the transmembrane module of the CSR comprises one or more transmembrane domains derived from CD28, CD30, CD3ε, CD3ζ, CD45, CD4, CD5, CD8, CD9, CD16, CD22, CD27, CD33, CD37, CD64, CD80, CD86, OX40 (CD134), 4-1BB (CD137), CD154, or ICOS. In some embodiments, the co-stimulatory immune cell signaling module is derived from the intracellular domain of a co-stimulatory receptor of a TCR. In some embodiments, the co-stimulatory receptor is selected from the group consisting of CD28, 4-1BB, OX40, ICOS, CD27, CD30, CD40, and DAP10. In some embodiments, the co-stimulatory receptor is CD28. In some embodiments, the co-stimulatory receptor is CD30.

In some embodiments according to any of the anti-WTMC constructs described above, the anti-WTMC construct is a conjugate comprising the antibody moiety and an effector molecule. In some embodiments, the effector molecule is a therapeutic agent selected from the group consisting of a drug, a toxin, a radioisotope, a protein, a peptide, and a nucleic acid. In some embodiments, the therapeutic agent is a drug or a toxin. In some embodiments, the effector molecule is a detectable label.

In some embodiments, there is provided an isolated nucleic acid or a set of isolated nucleic acids encoding the polypeptide component(s) of the anti-WTMC construct of any one of the anti-WTMC constructs described above.

In some embodiments, there is provided a vector or a set of vectors comprising the nucleic acid(s) encoding the polypeptide component(s) of the anti-WTMC construct of any one of the anti-WTMC constructs described above.

In some embodiments, there is provided a host cell comprising any one of the anti-WTMC constructs described above, the nucleic acid(s) encoding the polypeptide component(s) of any one of the anti-WTMC constructs described above, or the vector(s) comprising the nucleic acid(s) encoding the polypeptide component(s) of any of the anti-WTMC constructs described above.

In some embodiments, there is provided a method of producing any one of the anti-WTMC constructs described above, comprising culturing any of the host cells described above under conditions where the anti-WTMC construct is expressed, and recovering the anti-WTMC construct produced by the host cell.

In some embodiments, there is provided an effector cell expressing any of the anti-WTMC constructs described above.

In some embodiments, there is provided an effector cell expressing any one of the anti-WTMC caTCRs described above. In some embodiments, the effector cell further expresses a CSR comprising: i) a target-binding domain that specifically binds a target; ii) a transmembrane module; and iii) a co-stimulatory immune cell signaling module that is capable of providing a co-stimulatory signal to the effector cell, wherein the target-binding domain and the co-stimulatory immune cell signaling module are not derived from the same molecule, and wherein the CSR lacks a functional primary immune cell signaling domain of a TCR-associated T cell activation molecule selected from the group consisting of CD3ζ (TCRζ), FcRγ, FcRβ, CD3γ, CD3δ, CD3ε, CD5, CD79a, CD79b, and CD66d.

In some embodiments, the CSR specifically binds CD33. In some embodiments, the CSR specifically binds CD33, which is different from the WT1/MHC complex bound by the caTCR. In some embodiments, the CSR specifically binds a target that is identical to the WT1/MHC complex bound by the caTCR. In some embodiments, the CSR specifically binds a target that is not a WTMC. In some embodiments, the target bound by the CSR is expressed on a cancer cell. In some embodiments, the target bound by the CSR is expressed on a cancer cell selected from among chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma.

In some embodiments, the target bound by the CSR is expressed on a WT1-positive cancer cell and is CD33, CD371, CD123, or CD15. In some embodiments, the CSR specifically binds CD33, CD371, CD123, or CD15. In certain embodiments, the CSR specifically binds both CD33 and any one of CD371, CD123, and CD15. In some embodiments, the CSR specifically binds HER2 or MUC16.

In some embodiments, there is provided an effector cell expressing any one of the anti-WTMC CARs described above. In some embodiments, the effector cell further expresses a CSR comprising: i) a target-binding domain that specifically binds a target; ii) a transmembrane module; and iii) a co-stimulatory immune cell signaling module that is capable of providing a co-stimulatory signal to the effector cell, wherein the target-binding domain and the co-stimulatory immune cell signaling module are not derived from the same molecule, and wherein the CSR lacks a functional primary immune cell signaling domain of a TCR-associated T cell activation molecule selected from the group consisting of CD3ζ, TCRζ, FcRγ, FcRβ, CD3γ, CD3δ, CD3ε, CD5, CD22, CD79a, CD79b, and CD66d. In some embodiments, the CSR specifically binds a WTMC. In some embodiments, the CSR specifically binds a WTMC that is different from the WTMC bound by the CAR. In some embodiments, the CSR specifically binds a WTMC that is identical to the WTMC bound by the CAR. In some embodiments, the CSR specifically binds a target that is not a WTMC. In some embodiments, the target bound by the CSR is expressed on a cancer cell. In some embodiments, the target bound by the CSR is expressed on a cancer cell selected from among chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma. In some embodiments, the CSR specifically binds CD33, CD371, CD123, or CD15. In certain embodiments, the CSR specifically binds both CD33 and any one of CD371, CD123, and CD15. In some embodiments, the CSR specifically binds HER2 or MUC16.

In some embodiments, there is provided an effector cell expressing any one of the anti-WTMC CSRs described above, and wherein the effector cell further expresses a CAR comprising: (a) an extracellular domain that specifically binds a target; (b) a transmembrane domain; and (c) an intracellular signaling domain. In some embodiments, the CAR specifically binds a WTMC. In some embodiments, the CAR specifically binds a WTMC that is different from the WTMC bound by the CSR. In some embodiments, the CAR specifically binds a WTMC that is identical to the WTMC bound by the CSR. In some embodiments, the CAR specifically binds a target that is not a WTMC. In some embodiments, the target bound by the CAR is expressed on a cancer cell. In some embodiments, the target bound by the CAR is expressed on a cancer cell selected from among chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma. In some embodiments, the CSR specifically binds CD33, CD371, CD123, or CD15. In certain embodiments, the CSR specifically binds both CD33 and any one of CD371, CD123, and CD15. In some embodiments, the CSR specifically binds HER2 or MUC16.

In some embodiments, there is provided an effector cell expressing any one of the anti-WTMC CSRs described above, and wherein the effector cell further expresses a caTCR comprising: (a) an extracellular domain that specifically binds a target; and (b) a T cell receptor module (TCRM) comprising a first TCR domain (TCRD) comprising a first TCR transmembrane domain (TCR-TM) and a second TCRD comprising a second TCR-TM, wherein the TCRM is capable of associating with at least one TCR-associated signaling molecule. In some embodiments, the caTCR lacks a functional primary immune cell signaling domain of a TCR-associated T cell activation molecule selected from the group consisting of CD3ζ (TCRζ), FcRγ, FcRβ, CD3γ, CD3δ, CD3ε, CD5, CD22, CD79a, CD79b, and CD66d. Additionally or alternatively, in some embodiments, the caTCR lacks any primary immune cell signaling sequences. In some embodiments, the caTCR specifically binds a WTMC. In some embodiments, the caTCR specifically binds a WTMC that is different from CD33 that is bound by the CSR. In some embodiments, the caTCR specifically binds a WTMC that is identical to the target bound by the CSR. In some embodiments, the caTCR specifically binds a target that is not a WTMC. In some embodiments, the target bound by the caTCR is expressed on a cancer cell. In some embodiments, the target bound by the caTCR is expressed on a cancer cell selected from among chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma. In some embodiments, the CSR specifically binds CD33, CD371, CD123, or CD15. In certain embodiments, the CSR specifically binds both CD33 and any one of CD371, CD123, and CD15. In some embodiments, the CSR specifically binds HER2 or MUC16.

In some embodiments, the target bound by the caTCR is WT1/MHC complex, In some embodiments, there is provided an effector cell expressing any one of the anti-WT1 CSRs described above, the effector cell further expresses a TCR that specifically binds a complex comprising a peptide and an MHC protein. In some embodiments, the TCR specifically binds a WTMC. In some embodiments, the TCR specifically binds a target/MHC complex that is different from the WTMC bound by the CSR. In some embodiments, the TCR specifically binds a WTMC that is identical to the WTMC bound by the CSR. In some embodiments, the TCR specifically binds a target that is not a WTMC. In some embodiments, the target bound by the TCR is expressed on a cancer cell. In some embodiments, the target bound by the TCR is expressed on a cancer cell selected from among chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma.

In some embodiments, the target bound by the TCR is a WT1/MHC complex. In some embodiments, the target bound by the TCR is expressed on a cancer cell selected from among chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma.

In some embodiments, the TCR specifically binds a complex comprising a peptide and an MHC protein, and wherein the peptide is derived from WT1.

In some embodiments according to any of the effector cells described above, the effector cell is an immune cell. In some embodiments, the immune cell is a T cell. In some embodiments, the T cell is cytotoxic T cell, a helper T cell, a NKT cell, or a suppressor T cell. In some embodiments, the immune cell is a NK cell.

In some embodiments, there is provided a pharmaceutical composition comprising any of the anti-WTMC constructs described above, any of the nucleic acid(s) described above, any of the vector(s) described above, any of the host cells described above, or any of the effector cells described above, and a pharmaceutical acceptable carrier.

In some embodiments, there is provided a kit comprising any of the anti-WTMC constructs described above, any of the nucleic acid(s) described above, any of the vector(s) described above, any of the host cells described above, or any of the effector cells described above.

In some embodiments, there is provided a method of detecting WT1 in a sample, comprising contacting the sample with any of the anti-WTMC construct conjugates comprising the antibody moiety and an effector molecule described above, wherein the effector molecule is a label, and detecting the presence of the label.

In some embodiments, there is provided a method of treating an individual having a disease or disorder associated with expression, aberrant expression and/or aberrant activity of WT1, comprising administering to the individual an effective amount of any one of the anti-WTMC constructs described above, wherein the anti-WTMC construct is an antibody or antigen-binding fragment thereof, a multispecific antibody, a CAR, caTCR, a CSR, or an immunoconjugate. In some embodiments, the method further comprises administering to the individual an additional therapy.

In some embodiments, there is provided a method of treating an individual having a disease or disorder associated with expression, aberrant expression and/or aberrant activity of WT1, comprising administering to the individual an effective amount of any one of the effector cells described above, wherein the effector cell expresses an anti-WTMC CAR and a CSR. In some embodiments, the method further comprises administering to the individual an additional therapy.

In some embodiments, there is provided a method of treating an individual having a disease or disorder associated with expression, aberrant expression and/or aberrant activity of WT1, comprising administering to the individual an effective amount of any one of the effector cells described above, wherein the effector cell expresses an anti-WTMC caTCR and a CSR. In some embodiments, the method further comprises administering to the individual an additional therapy.

In some embodiments, there is provided a method of treating an individual having a disease or disorder associated with expression, aberrant expression and/or aberrant activity of WT1, comprising administering to the individual an effective amount of any one of the effector cells described above, wherein the effector cell expresses an anti-WTMC TCR and a CSR. In some embodiments, the method further comprises administering to the individual an additional therapy.

In some embodiments, there is provided a method of treating an individual having a disease or disorder associated with expression, aberrant expression and/or aberrant activity of WT1, comprising administering to the individual an effective amount of any one of the effector cells described above, wherein the effector cell expresses an anti-WTMC CSR and a CAR. In some embodiments, the method further comprises administering to the individual an additional therapy.

In some embodiments, there is provided a method of treating an individual having a disease or disorder associated with expression, aberrant expression and/or aberrant activity of WT1, comprising administering to the individual an effective amount of any one of the effector cells described above, wherein the effector cell expresses an anti-WTMC CSR and a caTCR. In some embodiments, the method further comprises administering to the individual an additional therapy.

In some embodiments, there is provided a method of treating an individual having a disease or disorder associated with expression, aberrant expression and/or aberrant activity of WT1, comprising administering to the individual an effective amount of any one of the effector cells described above, wherein the effector cell expresses an anti-WTMC CSR and a TCR. In some embodiments, the method further comprises administering to the individual an additional therapy.

In some embodiments, there is provided a method of diagnosing an individual having a disease or disorder associated with expression, aberrant expression and/or aberrant activity of WT1, comprising: a) administering an effective amount of any of the anti-WTMC construct conjugates comprising the antibody moiety and an effector molecule described above, wherein the effector molecule is a label, to the individual; and b) determining the level of the label in the individual, wherein a level of the label above a threshold level indicates that the individual has the disease or disorder associated with expression of WT1.

In some embodiments, there is provided a method of diagnosing an individual having a disease or disorder associated with expression, aberrant expression and/or aberrant activity of WT1, comprising: a) contacting a sample derived from the individual with any of the anti-WTMC construct conjugates comprising the antibody moiety and an effector molecule described above, wherein the effector molecule is a label; and b) determining the number of cells bound with the anti-WTMC construct in the sample, wherein a value for the number of cells bound with the anti-WTMC construct above a threshold level indicates that the individual has the disease or disorder associated with expression, aberrant expression, and/or aberrant activity of WT1.

In some embodiments, according to any of the methods of treating or diagnosing described above, the disease or disorder associated with expression, aberrant expression, and/or aberrant activity of WT1 is cancer. In some embodiments, the cancer is WT1-positive cancer. In some embodiments, the cancer is chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma.

In some embodiments, there is provided an anti-WTMC construct comprising an antibody moiety, wherein the antibody moiety comprises: (1) i) a V_{H} comprising the amino acid sequence of SEQ ID NO: 85; and ii) a V_{L} comprising the amino acid sequence of SEQ ID NO: 86; or (2) i) a V_{H} comprising the amino acid sequence of SEQ ID NO: 87; and ii) a V_{L} comprising the amino acid sequence of SEQ ID NO: 88; or (3) i) a V_{H} comprising the amino acid sequence of SEQ ID NO: 89; and ii) a V_{L} comprising the amino acid sequence of SEQ ID NO: 90; or (4) i) a V_{H} comprising the amino acid sequence of SEQ ID NO: 91; and ii) a V_{L} comprising the amino acid sequence of SEQ ID NO: 92; or (5) i) a V_{H} comprising the amino acid sequence of SEQ ID NO: 93; and ii) a V_{L} comprising the amino acid sequence of SEQ ID NO: 94; or (6) i) a V_{H} comprising the amino acid sequence of SEQ ID NO: 95; and ii) a V_{L} comprising the amino acid sequence of SEQ ID NO: 96; or (7) i) a V_{H} comprising the amino acid sequence of SEQ ID NO: 97; and ii) a V_{L} comprising the amino acid sequence of SEQ ID NO: 98; or (8) i) a V_{H} comprising the amino acid sequence of SEQ ID NO: 99; and ii) a V_{L} comprising the amino acid sequence of SEQ ID NO: 100; or (9) i) a V_{H} comprising the amino acid sequence of SEQ ID NO: 101; and ii) a V_{L} comprising the amino acid sequence of SEQ ID NO: 102; or (10) i) a V_{H} comprising the amino acid sequence of SEQ ID NO: 103; and ii) a V_{L} comprising the amino acid sequence of SEQ ID NO: 104; or (11) i) a V_{H} comprising the amino acid sequence of SEQ ID NO: 105; and ii) a V_{L} comprising the amino acid sequence of SEQ ID NO: 106; or (12) i) a V_{H} comprising the amino acid sequence of SEQ ID NO: 107; and ii) a V_{L} comprising the amino acid sequence of SEQ ID NO: 108; or (13) i) a V_{H} comprising the amino acid sequence of SEQ ID NO: 109; and ii) a V_{L} comprising the amino acid sequence of SEQ ID NO: 110; or (14) i) a V_{H} comprising the amino acid sequence of SEQ ID NO: 111; and ii) a V_{L} comprising the amino acid sequence of SEQ ID NO: 112.

In some embodiments, there is provided a method of killing a target cell that expresses a complex comprising WT1 and an MHC class I protein, the method comprising contacting the target cell an effective amount of an anti-WTMC construct according to any one of the anti-WTMC constructs described above or any of the effector cells described above. In some embodiments, the target cell is a cancer cell. In some embodiments, the cancer cell is a leukemia cell. In some embodiments, the cancer cell is a solid tumor.

Also provided are methods of making any of the anti-WTMC constructs described herein, articles of manufacture, and kits that are suitable for the methods described herein.

### BRIEF DESCRIPTION OF THE DRAWINGS

Various aspects of the disclosure are set forth with particularity in the appended claims. The file of this patent contains at least one drawing/photograph executed in color. Copies of this patent with color drawing(s)/photograph(s) will be provided by the Office upon request and payment of the necessary fee. A better understanding of the features and advantages of the present disclosure will be obtained by reference to the following detailed description that sets forth illustrative embodiments, in which the principles of the disclosure are utilized, and the accompanying drawings of which:

FIGS. 1A-1D show binding of the anti-WT1 peptide/MHC TCRm (a.k.a., ESK2) clone #18 and #34 and epitope specificity. FIG. 1A shows binding of anti-WT1 BiTE clones to T2 cells pulsed with or without indicated peptides. WT1 RMF or mutant peptides at a concentration of 50 g/ml were pulsed onto T2 cells overnight. Cells were washed and stained with BiTEs of ESK2 clone #18, #34 or ESK1 at 1 μg/ml. T2 cells alone or pulsed with irrelevant peptide HPV-E7(39) were used as controls. FIG. 1B shows HLA-A2 expression as determined by staining the cells with the anti-HLA-A2 mAb BB7 clone. FIG. 1C shows alanine screening by anti-WT1 BiTE clones. WT1 RMF sequences were substituted with alanine at positions 1, 3, 4, 5, 7, 8, or with glycine at position 6 indicated as WT1-A1 to WT1-A8 or WT1-G6 (FIG. 14) and the binding of ESK2-clones #18 and #34 (FIG. 1C) was determined by flow cytometric analysis. FIG. 1D shows stabilization of HLA on the surface of T2 cells after incubation with the alanine substituted peptides disclosed in FIG. 1C as measured by BB7 antibody to HLA-A2. The data are representative results from six similar experiments. For FIGS. 1A, 1C—the top-bottom order of the legend reflect the order of the columns from left-right.

FIGS. 2A-2F show specific recognition of tumor cells by Clone #34 BiTEs. Recognition and cytolytic activity of the naturally presented WT1 RMF/A2 complex on the tumor cell surface by the ESK2-clone #34 BiTE was probed. Mac-1 T cell lymphoma (FIG. 2A), JMN mesothelioma (FIG. 2B) or SW-620 colon cancer (FIG. 2C) cell lines were incubated with PBMC at an E:T ratio of 20:1, in the presence or absence of clone #34 BiTE at the indicated concentrations overnight, and the cytotoxicity was measured by BLI. BV173 CLL (FIG. 2D), SET-2 AML (FIG. 2E), or HL-60 AML cell lines (FIG. 2F) were incubated with PBMC at an E:T ratio of 20:1, in the presence or absence of clone #34 BiTE at the concentrations of 1 μg, 0.3 μg or 0.1 μg/ml for 5 hours and the cytotoxicity was measured using a ⁵¹Cr-release assay. The mean shown is the average of triplicate microwells plus/minus SD. The data are representative of ten experiments. The effector cells were used from several different donors; while differences among the experimental groups were similar, the baselines were variable among the individuals and therefore representative data only are shown. For FIGS. 2A-2C—ESK1 BiTE is the top most lines, #34 BiTE is the second top most line, and control is the most bottom line. For FIGS. 2D-2F-#34 BiTE is the top most line and control is the bottom line.

FIGS. 3A-3D show cytotoxicity of T cells comprising ESK-2 caTCR clones and an anti-CD33 costimulatory signaling receptor (CSR) against AML cells. T cells comprising (i) an anti-CD33 CSR and (ii) ESK-2 caTCR clones #18 (FIG. 3A), and #34 (FIG. 3B) were incubated with the indicated leukemia target cells at an E:T ratio of 1:1 overnight. FIG. 3C: T cells comprising (i) an anti-CD33 CSR and (ii) ESK-2 caTCR clone #34 was incubated with PBMCs of HLA-A2 positive or negative healthy donors at an E:T ratio of 1:1 overnight. A direct comparison of both clones and the control in another experiment is shown in FIG. 3D. The cytotoxicity was measured by LDH assay. Each data point was the average of triplicate cultures and representative of three similar experiments.

FIGS. 4A-4F show cytotoxicity mediated by “two signals” of T cells comprising ESK-2 caTCR clones and an anti-CD33 CSR. FIG. 4A: A cartoon depicting the design of the AbTCR and the costimulatory receptor only “CSR,” which together form the AbTCR-CSR. AbTCR is signal #1. CSR is signal #2. FIGS. 4B-4C: ESK2 AbTCR, or ESK2 AbTCR-CSR T cells were incubated with the indicated leukemia target cells at an E:T ratio of 1:1 overnight. The cytotoxicity was measured by luciferase-based assay. Each data point was the average of triplicate cultures+/−SD and representative of three similar experiments with different donors. To test if the cytotoxicity of AbTCR-CSR T cells required the primary signal, AML-14 (FIG. 4D), HL-60 (FIG. 4E) or SKOV3/A2 (FIG. 4F) cell lines were labeled with CFSE, washed and incubated with mock-T cells (untransduced), anti-CD33 CSR “costim only” T cells with no ESK2 (anti-WT1/MHC caTCR) but a non-relevant scFv as the recognition receptor, T cells with anti-CD33 CSR and ESK-2 caTCR clone #18, or T cells with anti-CD33 CSR and ESK-2 caTCR clone #34 at an E:T ratio of 1:1 overnight. The cells were harvested, washed and stained with mAb to CD33 and subjected to flow cytometry. The analysis was done by gating on larger tumor cells based on forward and side scatters and the percentage of CD33+ cells was shown in CFSE target population. For FIGS. 4B, 4C—the top-bottom order of the legend reflect the order of the columns from left-right.

FIGS. 5A-5E demonstrate that no cytotoxicity against normal PBMCs were observed with T cells comprising ESK-2 caTCR clones and an anti-CD33 CSR. Mock-T cells, anti-CD33 CSR “costim only” T cells (irrelevant scFv and no ESK2), T cells with anti-CD33 CSR and ESK-2 caTCR clone #18, or T cells with anti-CD33 CSR and ESK-2 caTCR clone #34 were incubated with CFSE-labeled AML-14 (FIG. 5A) (as a positive control), PBMCs from HLA-A2 positive donor (FIG. 5B) or negative donor (FIG. 5C) at an E:T ratio of 1:1 overnight. The cells were harvested, washed and stained with mAb to CD33 and Zombie dye (to stain dead cells) and analyzed by flow cytometry. The flow plots show a representative profile of CD33 vs CFSE double positive cells; the percentage CD33+ cell death was summarized in FIG. 5D. In the same experiments, CFSE+ target PBMCs were also stained with CD3 and CD19 and the percentage of each lineage cells were shown in bar graphs described in FIG. 5E. The data represent one of four separate experiments with different donors. For FIGS. 5D, 5E—the top-bottom order of the legend reflect the order of the columns from left-right.

FIGS. 6A-6D demonstrate that no cytotoxicity against neutrophils from normal donors were observed with T cells comprising ESK-2 caTCR clones and anti-CD33 CSR. Neutrophils from HLA-A*02:01 positive donor (FIG. 6A) or HLA-A*02:01 negative donor (FIG. 6B) were isolated using a human whole blood neutrophil isolation kit (Miltenyi) and incubated with mock-T cells, “costim only” T cells (irrelevant scFv and no ESK2), T cells with anti-CD33 CSR and ESK-2 caTCR clone #18, or T cells with anti-CD33 CSR and ESK-2 caTCR clone #34 at an E:T ratio of 1:1 overnight. The cells were stained with CD15 (neutrophils) or CD3 (T cells comprising ESK-2 caTCR clones and anti-CD33 CSR). The percentage of CD15+CFSE+ cells after co-culture are shown in FIG. 6C and the positive control AML-14 is shown in FIG. 6D. Since AML-14 cells do not express CD15, CD33 was used as its marker. The data represents one set of results from four independent donors. For FIG. 6C—the top-bottom order of the legend reflect the order of the columns from left-right.

FIGS. 7A-7C show therapeutic trials of T cells comprising ESK-2 caTCR constructs and anti-CD33 CSR in AML-14 xenograft animal models. AML-14 AML cells (5 million) were injected intravenously into NSG mice and leukemia engraftment was confirmed on day 13 by bioluminescent imaging (FIG. 7A). Control mock-T cells, T cells with anti-CD33 CSR and ESK-2 caTCR clone #18, or T cells with anti-CD33 CSR and ESK-2 caTCR clone #34 (6 million/mouse) were injected i.v., and tumor burden was monitored by bioluminescent imaging on day 18 and 21. Tumor burden (FIG. 7B) was calculated by summing the luminescent signal of each mouse and average signal for each group (n=5 per group). Survival of mice from experiment groups and the statistics of T cells with anti-CD33 CSR and ESK-2 caTCR clone #18, or T cells with anti-CD33 CSR and ESK-2 caTCR clone #34 over two control groups was calculated using the Mantal-Cox test (FIG. 7C).

FIGS. 8A-8C show therapeutic trials of T cells comprising ESK-2 caTCR constructs and anti-CD33 CSR in an OCI-AML-2 AML animal model. OCI-AML-2 cells (0.5 million) were injected intravenously into NSG mice. Groups were blindly assigned to treatment groups. Mock T cells, costim only T cells, or T cells comprising ESK-2 caTCR constructs and anti-CD33 CSR (one million) were injected i.v. on day 4 and day 12 post tumor cell injection. Tumor burden was assessed by BLI on the indicated days (FIG. 8A). Tumor burden was calculated by summing the luminescent signal of each mouse and average signal for each group (n=4 per group) was plotted (FIG. 8B). Survival of mice from experimental groups was shown and the statistics of T cells with anti-CD33 CSR and ESK-2 caTCR clone #18, or T cells with anti-CD33 CSR and ESK-2 caTCR clone #34 over three control groups was determined by Mantal-Cox test (FIG. 8C).

FIG. 9 shows binding kinetics of various concentrations of ESK2 BiTE antibodies to HLA-A2-WT1 RMF complex measured by Surface Plasmon Resonance (SPR).

FIGS. 10A-10G show characterization of ESK2 clone #34 BiTEs of the present technology. Whole blood from an HLA-A2 positive healthy donor (FIG. 10A) was stained with CD15 (neutrophil marker) vs isotype control (FIG. 10B) or ESK2-clone #34 mouse IgG1 (FIG. 10C) at 3 μg/ml; the percentage of CD15 vs ESK2-clone #34 was shown in neutrophil gates. Similarly, PBMCs from an HLA-A2 positive healthy donor were stained with anti-CD33 mAb vs isotype or clone #34 (FIGS. 10D-10E), or CD19 mAb vs isotype or #34 (FIGS. 10F-10G). The data are representative of three separate experiments with multiple donors.

FIGS. 11A-11F show identification and characterization of caTCR specific for WT1 RMF/HLA-A*02:01. FIG. 11A (top): Fourteen ESK2 caTCR clones were introduced into primary T cells and tested for in vitro cytotoxicity and specificity in a 16-hour co-culture with T2 cells loaded with RMF or other control peptides (irrelevant peptides or potential off-targets). FIG. 11A (bottom) demonstrates that ESK2 clones show specificity to RMF Mini-gene (MG) expressing cells. Eight ESK2 caTCR T cell clones were characterized using stable target cell lines ectopically expressing the RMF minigene (MG) using an in vitro cytotoxicity assay. Killing of cell lines HepG2 or SK-Hep1 (WT-) cells were compared to cells expressing the RMF minigene using an overnight LDH assay.

FIG. 11B: Eight ESK2 caTCR T cells were further screened for cytotoxicity by co-culture with an artificial SK-Hep1-WT1 MG cell line ectopically expressing RMF peptide region (RMF mini-gene, MG) in HLA-A*02:01-positive SK-Hep1. FIG. 11C: ESK2 caTCR clone 2, 15, 18 and 34 were selected and co-cultured with natural AML cell line SET-2 and SKM-1 which are WT1-positive and HLA-A*02:01-positive. After 16-hour co-culture, caTCR clone 18 and 34 showed significant killing activity against SKM-1 and modest activity against SET-2. Meanwhile, neither clone showed cytotoxicity against control cell line K562, which is WT1-positive and HLA-A*02:01-negative. FIG. 11D: The expression level of CD28 ligands CD80, CD86 or 41BB ligand CD137L on SET-2 and SKM-1 were measured by flow cytometric analysis. FIG. 11E: ESK2 caTCR 18 and 34 were co-cultured with SET-2 or SKM-1, with or without anti-human CD28 mAb (2 μg/ml) for external co-stimulatory signal, and the cytotoxicity against SET-2 was significantly improved. FIG. 11F shows killing activities and IFN-gamma release of ESK2 caTCR T cells+anti-hCD28 antibody hCD28. Selected ESK2 clones 18 and 34 caTCR T cells were tested for specific killing of WT1⁺/HLA-02⁻ K562 cells and in WT1+/HLA-02⁺ SET-2 cell lines. The ESK2 clone T cells were further tested in WT+ SKM-1 cells for killing and IFN-gamma release in the presence or absence of a costimulatory anti-CD28 antibody. For FIG. 11A, 11F—the top-bottom order of the legend reflect the order of the columns from left-right.

FIG. 12 shows activation of T cells comprising ESK2 caTCR clones 18 and 34, with or without co-expressing anti-CD33 CSR. T cell activation was assessed by IFN-γ release in the supernatants after 16-hour co-culture with SET-2, using IFN-γ ELISA. For FIG. 12—the top-bottom order of the legend reflect the order of the columns from left-right.

FIGS. 13A-13D show characterization of T cells comprising anti-CD33 CSR and ESK2 caTCR clones 18 and 34. Colony forming unit assays were done with AML-14 cell line as a positive control (FIG. 13A) and cord blood isolated CD34 positive HSCs from an HLA-A2 positive donor (FIG. 13B). Cells were plated at 1:1 co-culture of T cells comprising anti-CD33 CSR and ESK2 caTCR clones 18 and 34 and target cells. CD34 vs HLA-A2 staining before isolation of CD34+ cells and HLA-A2 positive cells in total HSC population is shown in FIGS. 13C-13D. Data represents one of two experiments. For FIG. 13A-13B—the top-bottom order of the legend reflect the order of the columns from left-right.

FIG. 14 shows peptide sequences (SEQ ID NOs: 113, and 115-121, 114 and 122 in order of appearance) used in the alanine mapping studies. Alanine or glycine-mutated peptides were named based on the position where the substitution was made. The WT1-AAA peptide was used to select for the antibody clones that recognize middle amino acids of the RMF peptide. Light font denotes the mutated amino acid. An irrelevant HLA-A2-binding peptide derived from HPV-E7 was used as a negative control. Position 2 and 9 are anchor residues for HLA and therefore were left intact.

FIG. 15 shows tumor cell lines and normal hematopoietic cells used for characterization of T cells comprising anti-CD33 CSR and ESK2 caTCR clones of the present technology. Determination of the phenotype of the cells was described in Table 2. PBMC: peripheral blood mononuclear cells.

FIG. 16 shows trans activation killing by AbTCR-CSR. Mock T Cells, AbTCR-CSR clone #18, or clone #34) were incubated with two different adherent targets (WT1+/HLA-A2+/CD33-JMN) and (WT1+/HLA-A2−/CD33−, MSTO), at an E:T ratio of 1:1 overnight. The suspension effector cells were then collected through aspiration and plated with AML-14 (WT1+/HLA-A2+/CD33+) and HL-60 target cells (WT1+/HLA-A2−/CD33+) at 1:1, 1:3 and 1:9 E:T ratios. After an overnight coculture, cytotoxicity was measured with a luciferase assay. The data points are averaged from triplicate wells+/−SD. The data shown are one of two separate experiments.

FIGS. 17A-17E show transgene design and surface expression of AbTCR-CSR. FIG. 17A: Schematic diagram of the lentiviral vector expressing AbTCR-CSR transgenes. The heavy and light chain of ESK2 Fab are fused to partial TCR delta and gamma chain to form Ab-TCR, while anti-CD33 scfv is linked to truncated CD28 as CSR. SP: signal peptide; S: spacer. FIG. 17B: Representative flow cytometry analysis of surface expression of AbTCR clones and CSR in primary T cells, detected by anti-Fab and anti-Myc staining respectively. FIG. 17C: Transduction rates of AbTCR and CSR in primary T cells from five donors. FIG. 17D: Representative expansion rate of AbTCR-CSR and un-transduced Mock T cells. FIG. 17E: Expansion of AbTCR-CSR and Mock T cells were derived from five donors. For FIG. 17E—the top-bottom order of the legend reflect the order of the columns from left-right.

FIGS. 18A-18K show representative flow plot showing total reduction of WT1+/HLA-A2+/CD33+ primary AML cells by AbTCR and AbTCR-CSR T cells. Primary AML samples were labeled with far-red, and co-incubated with control Mock-T cells (FIGS. 18A-18B), ESK2 Ab-TCR #18 (FIGS. 18C-18D), #34 (FIGS. 18E-18F) or AbTCR-CSR T cells #18 (FIGS. 18G-18H), #34 (FIGS. 18I-18J) at an E: T ratio of 1:1 for overnight. The cells were harvested and were stained with anti-CD33 mAb and analyzed by flow cytometry. The cells were gated on live cells and the same gates were applied to all groups. The percentage of CD33 and far-red double positive cells in the live cell gates were analyzed and the percentage of total CD33+/far-red+ were calculated (including CD33^{low} cells in AbTCR-CSR groups that show down modulation of CD33). FIG. 18K: WT1 RMF expression is shown by staining the cells with ESK1, ESK2 or isotype control mouse IgG conjugated with PE at 3 μg/ml.

### DETAILED DESCRIPTION

It is to be appreciated that certain aspects, modes, embodiments, variations and features of the present methods are described below in various levels of detail in order to provide a substantial understanding of the present technology. It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

The present application provides constructs (referred to herein as “anti-WTMC constructs”) that comprise an antibody moiety (referred to herein as an “anti-WTMC antibody moiety”) that specifically binds to a complex comprising WT1 peptide (e.g., WT1-RMF) and a major histocompatibility (MHC) class I protein, (referred to herein as a “WT1/MHC class I complex,” “WTMC”, or “WTMC complex”).

Using phage display technology, multiple monoclonal antigen-binding antibody fragments that are specific and high affinity against a WT1 peptide (e.g., WT1-RMF)/HLA-A*02:01 complex were generated. Flow cytometry assays demonstrated that the antibodies specifically recognized WT1 peptide (e.g., WT1-RMF)-pulsed T2 cells. The data presented herein demonstrate that antibodies against WT1 peptides in the context of an HLA complex can be effective therapeutic agents for a cancer characterized by aberrant expression of a target protein (such as WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma).

The anti-WTMC constructs allow for specific targeting of cells expressing WTMC, such as disease cells expressing (or overexpressing) WT1. When present in a chimeric antigen receptor (CAR) or chimeric antibody-T cell receptor construct (caTCR) expressed by a T cell, the anti-WTMC antibody moiety specifically redirects human T cells to kill target cells (e.g., cancer cells) expressing WTMC. Furthermore, when fused to a detectable label, the anti-WTMC antibody moiety may be used to visualize changes in the number and localization of WT1-expressing cells. Such information can, in turn, be used to diagnose and/or prognose target-associated diseases or disorders.

The present application provides constructs comprising an antibody moiety that specifically binds to a WTMC complex. Exemplary constructs include, but are not limited to, e.g., full-length anti-WTMC antibodies, multispecific anti-WTMC constructs (such as a bispecific anti-WTMC antibodies), anti-WTMC chimeric antigen receptors (“CARs”), anti-WTMC chimeric antibody-T cell receptor constructs (caTCRs), anti-WTMC chimeric signaling receptors (CSRs), an anti-WTMC immunoconjugates, as well as other constructs, as described in further detail below. Each of the anti-WTMC constructs described herein demonstrates high specificity for MHC-restricted human WT1 peptide (e.g., WT1-RMF).

The present application also provides nucleic acids that encode the anti-WTMC constructs described herein (or the polypeptide portion(s) thereof).

Also provided herein are compositions (such as pharmaceutical compositions or formulations) comprising an anti-WTMC construct described herein, or an effector cell expressing or associated with anti-WTMC construct described herein (such as a T cell expressing an anti-WTMC CAR, an anti-WTMC caTCR, or an anti-WTMC chimeric signaling receptor (CSR)).

The present application also provides methods of making and using the anti-WTMC constructs (or effector cells expressing or associated with the anti-WTMC constructs) for treatment, for diagnostic purposes, for prognostic purposes, and for inclusion into kits and articles of manufacture useful for the treatment, diagnosis, and/or prognosis of WT1-associated diseases and disorders.

Disclosed herein is the development of a second-generation anti-WT1 peptide/MHC TCRm (ESK2), which exhibited improved target specificity both in the BiTE® format and the caTCR T cell format.

### Definitions

Before describing the disclosed embodiments in detail, it is to be understood that the present disclosure is not limited to particular compositions or biological systems, which can, of course, vary. It is also to be understood that the terminology used herein is for the purpose of describing particular embodiments only and is not intended to be limiting.

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers.

As used herein “Wilm's Tumor 1 peptide” or “WT1” refers to any naturally occurring WT1 (e.g., WT1-RMF) from any vertebrate source, including mammals such as primates (e.g., humans, non-human primates (e.g., cynomolgus or rhesus monkeys)) and rodents (e.g., mice and rats), unless otherwise indicated. The term also encompasses naturally occurring variants of WT1 (e.g., WT1-RMF), e.g., allelic variants, and isoforms. WT1 (e.g., WT1-RMF) may be a part of a complex comprising a major histocompatibility (MHC) protein (e.g., a MHC class I protein). A complex comprising WT1 (e.g., WT1-RMF) and an MHC class I protein is class I protein, referred to herein as “WTMC” or a “WTMC complex”. An exemplary amino acid sequence for human WT1 (e.g., WT1-RMF) is SEQ ID NO: 113. The term “an anti-WTMC antibody moiety” as used herein specifically refers to an antibody moiety that specifically binds to a complex comprising a WT1 peptide (e.g., WT1-RMF) and an MHC class I protein. In some embodiments, the antibody moiety specifically recognizes amino acid residues FPNA (SEQ ID NO: 349) of SEQ ID NO: 113. In some embodiments, the MHC class I protein is HLA-A*02:01.

As used herein, “treatment” or “treating” is an approach for obtaining beneficial or desired results, including clinical results. For purposes of the present application, beneficial or desired clinical results include, but are not limited to, one or more of the following: alleviating one or more symptoms resulting from the disease, diminishing the extent of the disease, stabilizing the disease (e.g., preventing or delaying the worsening of the disease), preventing or delaying the spread (e.g., metastasis) of the disease, preventing or delaying the recurrence of the disease, delay or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of cancer (such as, for example, tumor volume). The methods provided herein contemplate any one or more of these aspects of treatment.

“Activation,” as used herein in relation to T cells, refers to the state of a T cell that has been sufficiently stimulated to induce detectable cellular proliferation. Activation can also be associated with induced cytokine production, and detectable effector functions.

The term “antibody moiety” includes full-length antibodies and antigen-binding fragments thereof. A full-length antibody comprises two heavy chains and two light chains. The variable regions of the light and heavy chains are responsible for antigen binding. The variable regions in each chain generally comprise three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and heavy chains, respectively. Several of the major antibody classes are divided into subclasses such as IgG1 (γI heavy chain), IgG2 (γ2 heavy chain), IgG3 (γ3 heavy chain), IgG4 (γ4 heavy chain), IgAQ1 (α1 heavy chain), or IgA2 (α2 heavy chain).

The term “antigen-binding fragment” as used herein refers to an antibody fragment including, but not limited to, e.g., a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “epitope” as used herein refers to the specific group of atoms or amino acids on an antigen to which an antibody or antibody moiety binds. Two antibodies or antibody moieties may bind the same epitope (or overlapping epitopes) within an antigen if they exhibit competitive binding for the antigen.

As used herein, a first antibody moiety “competes” for binding to WTMC with a second antibody moiety when the first antibody moiety inhibits binding of the second antibody moiety to WTMC by at least about 50% (such as at least about any of 55%, 60%, 65%, 70%, 75%, 80%, 85%, 90%, 95%, 98% or 99%) in the presence of an equimolar concentration of the first antibody moiety, or vice versa. A high throughput process for “binning” antibodies based upon their cross-competition is described in PCT Publication No. WO 03/48731.

As use herein, the term “specifically binds,” “specifically recognizes” or “is specific for” refers to measurable and reproducible interactions (such as binding between a target and an antibody or an antibody moiety) that are determinative of the presence of the target in the presence of a heterogeneous population of molecules, including biological molecules. For example, an antibody or antibody moiety that specifically binds to a target (which can be an epitope) is an antibody or antibody moiety that binds the target with greater affinity, avidity, more readily, and/or with greater duration than it binds to other targets. In some embodiments, an antibody or antibody moiety that specifically binds to an antigen reacts with one or more antigenic determinants of the antigen (for example an epitope on WT1) with a binding affinity that is at least about 10 times its binding affinity for other targets.

An “isolated” anti-WTMC construct as used herein refers to an anti-WTMC construct that (1) is not associated with proteins found in nature, (2) is free of other proteins from the same source, (3) is expressed by a cell from a different species, or (4) does not occur in nature.

The term “isolated nucleic acid” as used herein is intended to mean a nucleic acid of genomic, cDNA, or synthetic origin or some combination thereof, which by virtue of its origin the “isolated nucleic acid” (1) is not associated with all or a portion of a polynucleotide in which the “isolated nucleic acid” is found in nature, (2) is operably linked to a polynucleotide which it is not linked to in nature, or (3) does not occur in nature as part of a larger sequence.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table A as a comparison.

TABLE A
CDR Definitions

| | Kabat¹ | Chothia² | MacCallum³ | IMGT⁴ | AHo⁵ |
|---|---|---|---|---|---|
| V_{H} CDR1 | 31-35 | 26-32 | 30-35 | 27-38 | 25-40 |
| V_{H} CDR2 | 50-65 | 53-55 | 47-58 | 56-65 | 58-77 |
| V_{H} CDR3 | 95-102 | 96-101 | 93-101 | 105-117 | 109-137 |
| V_{L} CDR1 | 24-34 | 26-32 | 30-36 | 27-38 | 25-40 |
| V_{L} CDR2 | 50-56 | 50-52 | 46-55 | 56-65 | 58-77 |
| V_{L} CDR3 | 89-97 | 91-96 | 89-96 | 105-117 | 109-137 |

¹Residue numbering follows the nomenclature of Kabat et al., J. Biol. Chem. 252: 6609-6616 (1977); Kabat et al., U.S. Dept. of Health and Human Services, “Sequences of proteins of immunological interest” (1991).
²Residue numbering follows the nomenclature of Chothia et al., J. Mol. Biol. 196: 901-917 (1987); Al-Lazikani B. et al., J. Mol. Biol., 273: 927-948 (1997).
³Residue numbering follows the nomenclature of MacCallum et al., J. Mol. Biol. 262: 732-745 (1996); Abhinandan and Martin, Mol. Immunol., 45: 3832-3839 (2008).
⁴Residue numbering follows the nomenclature of Lefranc M. P. et al., Dev. Comp. Immunol., 27: 55-77 (2003); and Honegger and Plückthun, J. Mol. Biol., 309: 657-670 (2001).
⁵Residue numbering follows the nomenclature of Honegger and Plückthun, J. Mol. Biol., 309: 657-670 (2001).

The term “chimeric antibodies” refer to antibodies in which a portion of the heavy and/or light chain is identical or homologous to corresponding sequences in antibodies derived from a particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit a biological activity of interest (e.g., binding to WTMC) (see U.S. Pat. No. 4,816,567; and Morrison et al., Proc. Natl. Acad. Sci. USA, 81:6851-6855 (1984)).

The term “semi-synthetic” in reference to an antibody or antibody moiety means that the antibody or antibody moiety has one or more naturally occurring sequences and one or more non-naturally occurring (i.e., synthetic) sequences or amino acids.

“Fv” is the minimum antibody fragment which contains a complete antigen-recognition and antigen-binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which permits the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing scFv fragments (see preceding paragraph) typically with short linkers (such as about 5 to about 10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a complementarity determining region (CDR) of the recipient are replaced by residues from a CDR of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance (e.g., affinity for the target antigen). In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

“Percent (%) amino acid sequence identity” with respect to the polypeptide and antibody sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

The terms “Fc receptor” or “FcR” are used to describe a receptor that binds to the Fc region of an antibody. In some embodiments, an FcR is one that binds an IgG antibody (a γ receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain (see review M. in Daëron, Annu. Rev. Immunol. 15:203-234 (1997)). The term includes allotypes, such as FcγRIIIA allotypes: FcγRIIIA-Phel58, FcγRIIIA-Val158, FcγRIIA-R131 and/or FcγRIIA-H131. FcRs are reviewed in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

The term “FcRn” refers to the neonatal Fc receptor (FcRn). FcRn is structurally similar to the proteins of the major histocompatibility complex (MHC) and consists of an α-chain noncovalently bound to β2-microglobulin. The multiple functions of the neonatal Fc receptor FcRn are reviewed in Ghetie and Ward (2000) Annu. Rev. Immunol. 18, 739-766. FcRn plays a role in the passive delivery of immunoglobulin IgGs from mother to young and the regulation of serum IgG levels. FcRn can act as a salvage receptor, binding and transporting pinocytosed IgGs in intact form both within and across cells, rescuing them from a default degradative pathway.

The “CH1 domain” of a human IgG Fc region (also referred to as “C1” of “H1” domain) usually extends from about amino acid 118 to about amino acid 215 (EU numbering system).

The “hinge region” is generally defined as stretching from Glu216 to Pro230 of human IgG1 (Burton, Molec. Immunol. 22:161-206 (1985)). Hinge regions of other IgG isotypes may be aligned with the IgG1 sequence by placing the first and last cysteine residues forming inter-heavy chain S—S bonds in the same positions.

The “CH2 domain” of a human IgG Fc region (also referred to as “C2” of “H2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilize the CH2 domain. Burton, Molec Immunol. 22:161-206 (1985).

The “CH3 domain” (also referred to as “C2” or “H3” domain) comprises the stretch of residues C-terminal to a CH2 domain in an Fc region (i.e., from about amino acid residue 341 to the C-terminal end of an antibody sequence, typically at amino acid residue 446 or 447 of an IgG).

A “functional Fc fragment” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding domain (e.g., an antibody variable domain) and can be assessed using various assays known in the art.

An antibody with a variant IgG Fc with “altered” FcR binding affinity or ADCC activity is one which has either enhanced or diminished FcR binding activity (e.g., FcγR or FcRn) and/or ADCC activity compared to a parent polypeptide or to a polypeptide comprising a native sequence Fc region. The variant Fc which “exhibits increased binding” to an FcR binds at least one FcR with higher affinity (e.g., lower apparent K_{d} or IC₅₀ value) than the parent polypeptide or a native sequence IgG Fc. According to some embodiments, the improvement in binding compared to a parent polypeptide is about 3-fold, such as about any of 5, 10, 25, 50, 60, 100, 150, 200, or up to 500-fold, or about 25% to 1000% improvement in binding. The polypeptide variant which “exhibits decreased binding” to an FcR, binds at least one FcR with lower affinity (e.g., higher apparent K_{d} or higher IC₅₀ value) than a parent polypeptide. The decrease in binding compared to a parent polypeptide may be about 40% or more decrease in binding.

“Antibody-dependent cell-mediated cytotoxicity” or “ADCC” refers to a form of cytotoxicity in which secreted Ig bound to Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

The polypeptide comprising a variant Fc region which “exhibits increased ADCC” or mediates ADCC in the presence of human effector cells more effectively than a polypeptide having wild type IgG Fc or a parent polypeptide is one which in vitro or in vivo is substantially more effective at mediating ADCC, when the amounts of polypeptide with variant Fc region and the polypeptide with wild type Fc region (or the parent polypeptide) in the assay are essentially the same. Generally, such variants will be identified using any in vitro ADCC assay known in the art, such as assays or methods for determining ADCC activity, e.g., in an animal model etc. In some embodiments, the variant is from about 5 fold to about 100 fold, e.g., from about 25 to about 50 fold, more effective at mediating ADCC than the wild type Fc (or parent polypeptide).

“Complement dependent cytotoxicity” or “CDC” refers to the lysis of a target cell in the presence of complement. Activation of the classical complement pathway is initiated by the binding of the first component of the complement system (Clq) to antibodies (of the appropriate subclass) which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences and increased or decreased C1q binding capability are described in U.S. Pat. No. 6,194,551B1 and WO99/51642. The contents of those patent publications are specifically incorporated herein by reference. See, also, Idusogie et al. J. Immunol. 164: 4178-4184 (2000).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may, in some version, contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared times 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

An “effective amount” of an anti-WTMC construct or composition as disclosed herein, is an amount sufficient to carry out a specifically stated purpose. An “effective amount” can be determined empirically and by known methods relating to the stated purpose.

The term “therapeutically effective amount” refers to an amount of an anti-WTMC construct or composition as disclosed herein, effective to “treat” a disease or disorder in an individual. In the case of cancer, the therapeutically effective amount of the anti-WTMC construct or composition as disclosed herein can reduce the number of cancer cells; reduce the tumor size or weight; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the anti-WTMC construct or composition as disclosed herein can prevent growth and/or kill existing cancer cells, it can be cytostatic and/or cytotoxic. In some embodiments, the therapeutically effective amount is a growth inhibitory amount. In some embodiments, the therapeutically effective amount is an amount that extends the survival of a patient. In some embodiments, the therapeutically effective amount is an amount that improves progression free survival of a patient.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

The term “label” when used herein refers to a detectable compound or composition which can be conjugated directly or indirectly to the anti-WTMC antibody moiety. The label may be detectable by itself (e.g., radioisotope labels or fluorescent labels) or, in the case of an enzymatic label, may catalyze chemical alteration of a substrate compound or composition which is detectable.

The term “specific binding” or “specifically binds to” or is “specific for” a particular polypeptide or an epitope on a particular polypeptide target as used herein can be exhibited, for example, by a molecule having a K_{D} for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or less. In some embodiments, the term “specific binding” refers to binding where a molecule binds to a particular complex (e.g., WTMC) or epitope on a particular complex (e.g., WT1 (e.g., WT1-RMF) or HLA-A*02:01) without substantially binding to any other polypeptide or polypeptide epitope.

The term “chimeric antigen receptor (CAR)”, as used herein, refers to an artificially constructed hybrid single-chain protein or single-chain polypeptide containing an extracellular target-binding (e.g., antigen-binding) domain, linked directly or indirectly to a transmembrane domain (“TM domain”, e.g., the transmembrane domain of a costimulatory molecule), which is in turn linked directly or indirectly to an intracellular signaling domain (ISD) comprising a primary immune cell signaling domain (e.g., one involved in T cell or NK cell activation). The extracellular target-binding domain can be a single-chain variable fragment derived from an antibody (scFv). In addition to scFvs, other single chain antigen binding domains can be used in CAR, e.g., tandem scFvs, single-domain antibody fragments (V_{H}Hs or sdAbs), single domain bispecific antibodies (BsAbs), intrabodies, nanobodies, immunokines in a single chain format, and Fab, Fab′, or (Fab′)₂ in single chain formats. The extracellular target-binding domain can be joined to the TM domain via a flexible hinge/spacer region. The intracellular signaling domain (ISD) comprises a primary signaling sequence, or primary immune cell signaling sequence, which can be from an antigen-dependent, TCR-associated T cell activation molecule, e.g., a portion of the intracellular domain of CD3ζ (TCRζ), FcRγ, FcRβ, CD3γ, CD3δ, CD3ε, CD5, CD79a, CD79b, or CD66d. The ISD can further comprise a costimulatory signaling sequence; e.g., a portion of the intracellular domain of an antigen-independent, costimulatory molecule such as CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds CD83, Dap10, or the like. Characteristics of CARs include their ability to redirect immune cell (e.g., T cell or NK cell) specificity and reactivity toward a selected target in either MHC-restricted (in cases of TCR-mimic antibodies) or non-MHC-restricted (in cases of antibodies against cell surface proteins) manners, exploiting the antigen-binding properties of monoclonal antibodies. The non-MHC-restricted antigen recognition gives immune cells (e.g., T cells or NK cells) expressing CARs the ability to recognize antigen independent of antigen processing, thus bypassing a major mechanism of tumor escape.

There are currently three generations of CARs. The “first generation” CARs are typically single-chain polypeptides composed of an scFv as the antigen-binding domain fused to a transmembrane domain fused to the cytoplasmic/intracellular domain, which comprises a primary immune cell signaling sequence such as the intracellular domain from the CD3ζ chain, which is the primary transmitter of signals from endogenous TCRs. The “first generation” CARs can provide de novo antigen recognition and cause activation of both CD4⁺ and CD8⁺ T cells through their CD3ζ chain signaling domain in a single fusion molecule, independent of HLA-mediated antigen presentation. The “second generation” CARs add intracellular domains from various costimulatory molecules (e.g., CD28, 4-1BB, ICOS, OX40) to the primary immune cell signaling sequence of the CAR to provide additional signals to the T cell. Thus, the “second generation” CARs comprise fragments that provide costimulation (e.g., CD28 or 4-IBB) and activation (e.g., CD3ζ). Preclinical studies have indicated that the “second generation” CARs can improve the antitumor activity of T cells.

The “third generation” CARs comprise those that provide multiple costimulation (e.g., CD28 and 4-1BB) and activation (e.g., CD3ζ). Examples of CAR T therapies are described, see, e.g., U.S. Pat. No. 10,221,245 describing CAR CTL019 which has an anti-CD33 extracellular target-binding domain, a transmembrane domain from CD8, a costimulatory domain from 4-1BB, and a primary signaling domain from CD3ζ, as well as U.S. Pat. No. 9,855,298 which describes a CAR having an anti-CD33 extracellular target-binding domain, a costimulatory domain from CD28, and a primary signaling domain from CD3ζ.

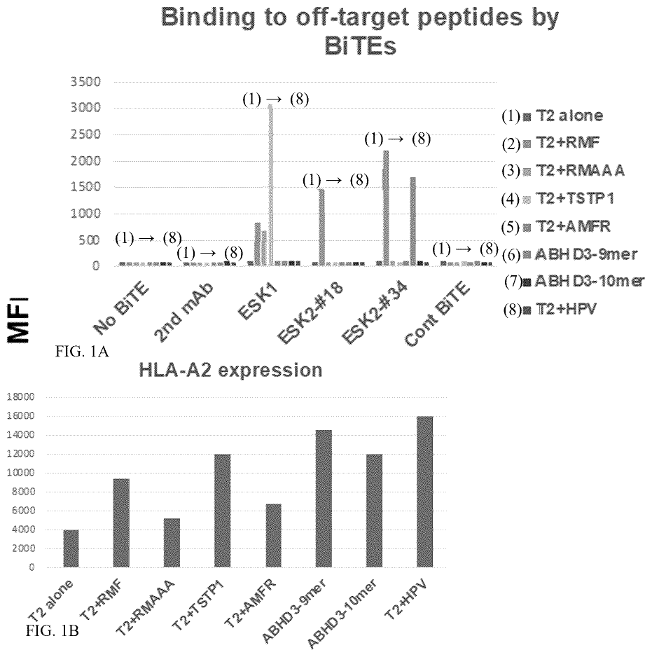

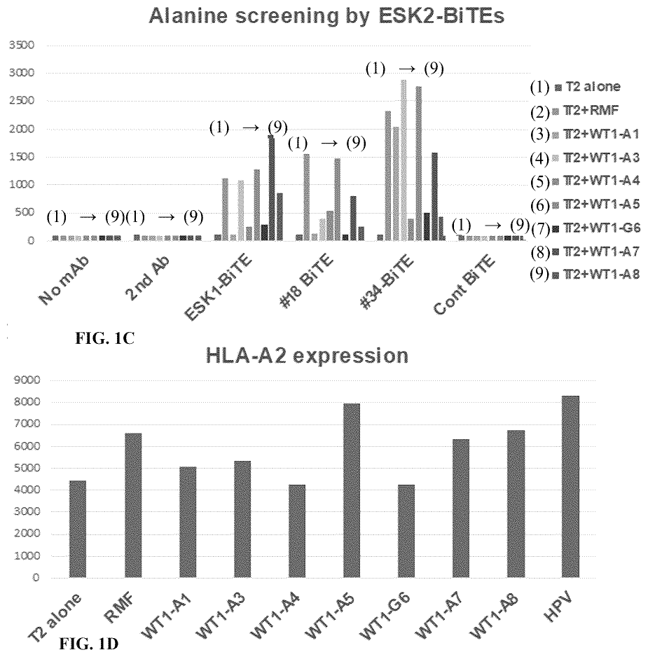

As used herein, the term “chimeric antibody-T cell receptor” or “caTCR” construct refers to a functional multi-chain polypeptide complex comprising one or more antibody moieties linked to a T cell receptor module (TCRM) derived from transmembrane domains and optionally intracellular domains of T cell receptor (TCR) chains, such as TCRα/β or TCRγ/δ chains. Upon binding of the one or more antibody moieties to the target(s), the TCRM associates with at least one TCR-associated signaling module, such as CD3γ, CD3δ, CD3ε, and/or CD3ζ. caTCRs can be monospecific or multispecific. Suitable antibody moieties include, but are not limited to, an Fv, a Fab, and an scFv. Various constructs of caTCRs have been described, for example, see U.S. Ser. No. 10/822,413B2 (e.g., FIG. 1), which is incorporated herein by reference in its entirety. In some embodiments, a caTCR comprises a first polypeptide chain comprising an antibody heavy chain variable region (V_{H}), a first transmembrane domain and a first intracellular domain derived from a first T cell receptor chain, and a second polypeptide chain comprising an antibody light chain variable region (V_{L}), a second transmembrane domain and a second intracellular domain derived from a second TCR chain, wherein the VH and the VL associate with each other to form an antigen binding domain that specifically binds a target. In some embodiments, the first polypeptide comprises a first antibody constant region (e.g., CH1) and the second polypeptide comprises a second antibody constant region (e.g., C_{L}). In some embodiments, the first polypeptide and the second polypeptide do not comprise antibody constant regions. In some embodiments, a caTCR does not comprise a primary immune cell signaling sequence (e.g., a portion of the intracellular domain of CD3γ, CD3δ, CD3ε, or CD3ζ). In some embodiments, a caTCR does not include a functional primary immune cell signaling sequence. In some embodiments, an anti-WTMC caTCR comprises a) an extracellular domain comprising an anti-WTMC antibody moiety that specifically binds to a complex comprising a WT1 peptide (e.g., WT1-RMF) (SEQ ID NO: 113) and a MHC class I protein, and b) a T cell receptor module (TCRM) capable of associating with at least one TCR-associated signaling module. In some embodiments, the antibody moiety specifically recognizes amino acid residues FPNA (SEQ ID NO: 349) of SEQ ID NO: 113. In some embodiments, the MHC class I protein is HLA-A*02:01. The terms “chimeric antibody-T cell receptor” (caTCR) and “antibody-T cell receptor” or “antibody/T-cell receptor” chimeric molecule or construct (abTCR or AbTCR) are used interchangeably herein. Further descriptions and examples of caTCR and abTCR may be found in, e.g., US 2019/0248865 and US 2021/0101954, the contents of which are incorporated by reference herein in their entirety.

The terms “chimeric stimulatory receptor” of “CSR” refers to an artificially constructed receptor comprising a target-binding domain linked directly or indirectly to a transmembrane, and an intracellular signaling domain derived from one or more co-stimulatory molecules of an immune cell, wherein the intracellular signaling domain does not comprise a primary immune cell signaling sequence or a functional primary immune cell signaling sequence, such as a CD3ζ signaling sequence or a functional CD3ζ signaling sequence. In some embodiments, the transmembrane domain of a CSR is not a transmembrane domain of a TCR or a derivative thereof. In nature, activation of effector immune cells (e.g., T cells) typically require a primary signal through antigen-specific receptors (e.g., TCR) on the effector immune cell and a secondary signal provided by the interaction between co-stimulatory molecules expressed on the membrane of antigen presenting cells and effector immune cells. Upon target binding, a CSR can send a secondary signal that regulates activation of immune cells, e.g., T cells. The target-binding domain in a CSR may be an antibody moiety or an extracellular domain of a receptor.

As used herein, “primary immune cell signaling sequences” refer to sequences that act in a stimulatory manner may contain signaling motifs which are known as immunoreceptor tyrosine-based activation motifs or ITAMs. Examples of ITAM-containing primary immune cell signaling sequences include those derived from CD3ζ (TCRζ), FcRγ, FcRβ, CD3γ, CD3δ, CD3ε, CD5, CD79a, CD79b, and CD66d. A “functional” primary immune cell signaling sequence is a sequence that is capable of transducing an immune cell activation signal when operably coupled to an appropriate receptor. “Non-functional” primary immune cell signaling sequences, which may comprise fragments or variants of primary immune cell signaling sequences, are unable to transduce an immune cell activation signal.

It is understood that embodiments of the present application described herein include “consisting of” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat cancer of type X means the method is used to treat cancer of types other than X.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the terms “subject”, “patient”, or “individual” can be an individual organism, a vertebrate, a mammal, or a human. In some embodiments, the subject, patient or individual is a human.

### Anti-WTMC Constructs

In one aspect, the present application provides WT1/MHC class I complex-specific constructs (anti-WTMC constructs) that comprise an antibody moiety that specifically binds to a complex comprising a WT1 peptide (e.g., WT1-RMF) and an MHC class I protein (“WT1/MHC class I complex,” or “WTMC”). In some embodiments, the anti-WTMC construct is an isolated anti-WTMC construct. The specificity of the anti-WTMC construct derives from an anti-WTMC antibody moiety, such as a full-length antibody or antigen-binding fragment thereof that specifically binds to the WTMC. In some embodiments, the WT1 peptide (e.g., WT1-RMF) comprises the amino acid sequence set forth in SEQ ID NO: 113. In some embodiments of the anti-WTMC construct, the antibody moiety specifically recognizes amino acid residues FPNA (SEQ ID NO: 349) of SEQ ID NO: 113. In some embodiments, the MHC class I protein is HLA-A*02:01.

Anti-WTMC constructs within the scope of the present application include, without limitation, e.g., full-length anti-WTMC antibodies, multispecific anti-WTMC constructs, anti-WTMC chimeric antigen receptors (CARs), anti-WTMC chimeric antibody-T cell receptor constructs (caTCRs), anti-WTMC chimeric signaling receptors (CSRs), anti-WTMC immunoconjugates, and others, as described herein below.

For example, in some embodiments, the anti-WTMC construct comprises an anti-WTMC antibody moiety that specifically binds to a WTMC complex (e.g., a complex comprising a WT1 peptide (e.g., WT1-RMF) and an MHC class I protein expressed on the surface of a cell, such as a cancer cell). In some embodiments, the extent of binding of the anti-WTMC antibody to a non-target polypeptide is less than about 10% of the binding of the anti-WTMC antibody moiety to a WTMC as determined by methods known in the art, such as ELISA, fluorescence activated cell sorting (FACS) analysis, or radioimmunoprecipitation (RIA). Specific binding can be measured, for example, by determining the binding of a molecule compared to the binding of a control molecule, which generally is a molecule of similar structure that does not have binding activity. For example, specific binding can be determined by competition with a control molecule that is similar to the target, for example, an excess of unlabeled target. In this case, specific binding is indicated if the binding of the labeled target to a probe is competitively inhibited by excess unlabeled target.

For example, in some embodiments, there is provided an anti-WTMC construct comprising an anti-WTMC antibody moiety that specifically binds to a complex comprising a WT1 peptide (e.g., WT1-RMF) and an MHC class I protein. In some embodiments, the WT1 peptide (e.g., WT1-RMF) comprises (such as consists of) the amino acid sequence of SEQ ID NO: 113. In some embodiments of the anti-WTMC construct, the antibody moiety specifically recognizes amino acid residues FPNA (SEQ ID NO: 349) of SEQ ID NO: 113. In some embodiments, the MHC class I protein is HLA-A02. In some embodiments, the MHC class I protein is HLA-A*02:01 (GenBank Accession No.: AAO20853). In some embodiments, the anti-WTMC construct is non-naturally occurring. In some embodiments, the anti-WTMC construct is a full-length antibody. In some embodiments, the anti-WTMC construct is a multispecific (such as bispecific) molecule. In some embodiments, the anti-WTMC construct is a chimeric receptor (e.g., a CAR, caTCR, CSR). In some embodiments, the anti-WTMC construct is an immunoconjugate. In some embodiments, the anti-WTMC construct binds the WTMC complex with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values).

In some embodiments, the anti-WTMC construct comprises any one of the anti-WTMC antibody moieties described in the “Anti-WTMC antibody moieties” section below. In some embodiments, the anti-WTMC construct is non-naturally occurring. In some embodiments, the anti-WTMC construct is a full-length antibody. In some embodiments, the anti-WTMC construct is a multispecific (such as bispecific) molecule. In some embodiments, the anti-WTMC construct is a chimeric receptor (e.g., a CAR, caTCR, CSR). In some embodiments, the anti-WTMC construct is an immunoconjugate.

In some embodiments, the anti-WTMC construct comprises an anti-WTMC antibody moiety that comprises: (a) a HC-CDR1 comprising an amino acid sequence set forth in SEQ ID NO: 1; (b) a HC-CDR2 comprising an amino acid sequence set forth in SEQ ID NO: 2; (c) a HC-CDR3 comprising an amino acid sequence set forth in SEQ ID NO: 3; (d) a LC-CDR1 comprising an amino acid sequence set forth in SEQ ID NO: 4; (e) a LC-CDR2 comprising an amino acid sequence set forth in SEQ ID NO: 5; and (f) a LC-CDR3 comprising an amino acid sequence set forth in SEQ ID NO: 6. In some embodiments, the CDRs are human CDRs. In some embodiments, the anti-WTMC construct comprises an anti-WTMC antibody moiety that comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 85 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 86. In some embodiments, the anti-WTMC construct comprises an anti-WTMC antibody moiety that comprises a V_{H} domain comprising SEQ ID NO: 85 and a V_{L} domain comprising SEQ ID NO: 86. An anti-WTMC antibody moiety comprising SEQ ID NO: 85 and SEQ ID NO: 86 is alternatively referred to herein as a “Clone 1 anti-WTMC antibody moiety”.

In some embodiments, the anti-WTMC construct comprises an anti-WTMC antibody moiety that comprises: (a) a HC-CDR1 comprising an amino acid sequence set forth in SEQ ID NO: 7; (b) a HC-CDR2 comprising an amino acid sequence set forth in SEQ ID NO: 8; (c) a HC-CDR3 comprising an amino acid sequence set forth in SEQ ID NO: 9; (d) a LC-CDR1 comprising an amino acid sequence set forth in SEQ ID NO: 10; (e) a LC-CDR2 comprising an amino acid sequence set forth in SEQ ID NO: 11; and (f) a LC-CDR3 comprising an amino acid sequence set forth in SEQ ID NO: 12. In some embodiments, the CDRs are human CDRs. In some embodiments, the anti-WTMC construct comprises an anti-WTMC antibody moiety that comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 87 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 88. In some embodiments, the anti-WTMC construct comprises an anti-WTMC antibody moiety that comprises a V_{H} domain comprising SEQ ID NO: 87 and a V_{L} domain comprising SEQ ID NO: 88. An anti-WTMC antibody moiety comprising SEQ ID NO: 87 and SEQ ID NO: 88 is alternatively referred to herein as a “Clone 2 anti-WTMC antibody moiety”.

In some embodiments, the anti-WTMC construct comprises an anti-WTMC antibody moiety that comprises: (a) a HC-CDR1 comprising an amino acid sequence set forth in SEQ ID NO: 13; (b) a HC-CDR2 comprising an amino acid sequence set forth in SEQ ID NO: 14; (c) a HC-CDR3 comprising an amino acid sequence set forth in SEQ ID NO: 15; (d) a LC-CDR1 comprising an amino acid sequence set forth in SEQ ID NO: 16; (e) a LC-CDR2 comprising an amino acid sequence set forth in SEQ ID NO: 17; and (f) a LC-CDR3 comprising an amino acid sequence set forth in SEQ ID NO: 18. In some embodiments, the CDRs are human CDRs. In some embodiments, the anti-WTMC construct comprises an anti-WTMC antibody moiety that comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 89 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 90. In some embodiments, the anti-WTMC construct comprises an anti-WTMC antibody moiety that comprises a V_{H} domain comprising SEQ ID NO: 89 and a V_{L} domain comprising SEQ ID NO: 90. An anti-WTMC antibody moiety comprising SEQ ID NO: 89 and SEQ ID NO: 90 is alternatively referred to herein as a “Clone 10 anti-WTMC antibody moiety”.

In some embodiments, the anti-WTMC construct comprises an anti-WTMC antibody moiety that comprises: (a) a HC-CDR1 comprising an amino acid sequence set forth in SEQ ID NO: 19; (b) a HC-CDR2 comprising an amino acid sequence set forth in SEQ ID NO: 20; (c) a HC-CDR3 comprising an amino acid sequence set forth in SEQ ID NO: 21; (d) a LC-CDR1 comprising an amino acid sequence set forth in SEQ ID NO: 22; (e) a LC-CDR2 comprising an amino acid sequence set forth in SEQ ID NO: 23; and (f) a LC-CDR3 comprising an amino acid sequence set forth in SEQ ID NO: 24. In some embodiments, the CDRs are human CDRs. In some embodiments, the anti-WTMC construct comprises an anti-WTMC antibody moiety that comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 91 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 92. In some embodiments, the anti-WTMC construct comprises an anti-WTMC antibody moiety that comprises a V_{H} domain comprising SEQ ID NO: 91 and a V_{L} domain comprising SEQ ID NO: 92. An anti-WTMC antibody moiety comprising SEQ ID NO: 91 and SEQ ID NO: 92 is alternatively referred to herein as a “Clone 11 anti-WTMC antibody moiety”.

In some embodiments, the anti-WTMC construct comprises an anti-WTMC antibody moiety that comprises: (a) a HC-CDR1 comprising an amino acid sequence set forth in SEQ ID NO: 25; (b) a HC-CDR2 comprising an amino acid sequence set forth in SEQ ID NO: 26; (c) a HC-CDR3 comprising an amino acid sequence set forth in SEQ ID NO: 27; (d) a LC-CDR1 comprising an amino acid sequence set forth in SEQ ID NO: 28; (e) a LC-CDR2 comprising an amino acid sequence set forth in SEQ ID NO: 29; and (f) a LC-CDR3 comprising an amino acid sequence set forth in SEQ ID NO: 30. In some embodiments, the CDRs are human CDRs. In some embodiments, the anti-WTMC construct comprises an anti-WTMC antibody moiety that comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 93 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 94. In some embodiments, the anti-WTMC construct comprises an anti-WTMC antibody moiety that comprises a V_{H} domain comprising SEQ ID NO: 93 and a V_{L} domain comprising SEQ ID NO: 94. An anti-WTMC antibody moiety comprising SEQ ID NO: 93 and SEQ ID NO: 94 is alternatively referred to herein as a “Clone 12 anti-WTMC antibody moiety”.

In some embodiments, the anti-WTMC construct comprises an anti-WTMC antibody moiety that comprises: (a) a HC-CDR1 comprising an amino acid sequence set forth in SEQ ID NO: 31; (b) a HC-CDR2 comprising an amino acid sequence set forth in SEQ ID NO: 32; (c) a HC-CDR3 comprising an amino acid sequence set forth in SEQ ID NO: 33; (d) a LC-CDR1 comprising an amino acid sequence set forth in SEQ ID NO: 34; (e) a LC-CDR2 comprising an amino acid sequence set forth in SEQ ID NO: 35; and (f) a LC-CDR3 comprising an amino acid sequence set forth in SEQ ID NO: 36. In some embodiments, the CDRs are human CDRs. In some embodiments, the anti-WTMC construct comprises an anti-WTMC antibody moiety that comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 95 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 96. In some embodiments, the anti-WTMC construct comprises an anti-WTMC antibody moiety that comprises a V_{H} domain comprising SEQ ID NO: 95 and a V_{L} domain comprising SEQ ID NO: 96. An anti-WTMC antibody moiety comprising SEQ ID NO: 95 and SEQ ID NO: 96 is alternatively referred to herein as a “Clone 14 anti-WTMC antibody moiety”.

In some embodiments, the anti-WTMC construct comprises an anti-WTMC antibody moiety that comprises: (a) a HC-CDR1 comprising an amino acid sequence set forth in SEQ ID NO: 37; (b) a HC-CDR2 comprising an amino acid sequence set forth in SEQ ID NO: 38; (c) a HC-CDR3 comprising an amino acid sequence set forth in SEQ ID NO: 39; (d) a LC-CDR1 comprising an amino acid sequence set forth in SEQ ID NO: 40; (e) a LC-CDR2 comprising an amino acid sequence set forth in SEQ ID NO: 41; and (f) a LC-CDR3 comprising an amino acid sequence set forth in SEQ ID NO: 42. In some embodiments, the CDRs are human CDRs. In some embodiments, the anti-WTMC construct comprises an anti-WTMC antibody moiety that comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 97 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 98. In some embodiments, the anti-WTMC construct comprises an anti-WTMC antibody moiety that comprises a V_{H} domain comprising SEQ ID NO: 97 and a V_{L} domain comprising SEQ ID NO: 98. An anti-WTMC antibody moiety comprising SEQ ID NO: 97 and SEQ ID NO: 98 is alternatively referred to herein as a “Clone 15 anti-WTMC antibody moiety”.

In some embodiments, the anti-WTMC construct comprises an anti-WTMC antibody moiety that comprises: (a) a HC-CDR1 comprising an amino acid sequence set forth in SEQ ID NO: 43; (b) a HC-CDR2 comprising an amino acid sequence set forth in SEQ ID NO: 44; (c) a HC-CDR3 comprising an amino acid sequence set forth in SEQ ID NO: 45; (d) a LC-CDR1 comprising an amino acid sequence set forth in SEQ ID NO: 46; (e) a LC-CDR2 comprising an amino acid sequence set forth in SEQ ID NO: 47; and (f) a LC-CDR3 comprising an amino acid sequence set forth in SEQ ID NO: 48. In some embodiments, the CDRs are human CDRs. In some embodiments, the anti-WTMC construct comprises an anti-WTMC antibody moiety that comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 99 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 100. In some embodiments, the anti-WTMC construct comprises an anti-WTMC antibody moiety that comprises a V_{H} domain comprising SEQ ID NO: 99 and a V_{L} domain comprising SEQ ID NO: 100. An anti-WTMC antibody moiety comprising SEQ ID NO: 99 and SEQ ID NO: 100 is alternatively referred to herein as a “Clone 17 anti-WTMC antibody moiety”.

In some embodiments, the anti-WTMC construct comprises an anti-WTMC antibody moiety that comprises: (a) a HC-CDR1 comprising an amino acid sequence set forth in SEQ ID NO: 49; (b) a HC-CDR2 comprising an amino acid sequence set forth in SEQ ID NO: 50; (c) a HC-CDR3 comprising an amino acid sequence set forth in SEQ ID NO: 51; (d) a LC-CDR1 comprising an amino acid sequence set forth in SEQ ID NO: 52; (e) a LC-CDR2 comprising an amino acid sequence set forth in SEQ ID NO: 53; and (f) a LC-CDR3 comprising an amino acid sequence set forth in SEQ ID NO: 54. In some embodiments, the CDRs are human CDRs. In some embodiments, the anti-WTMC construct comprises an anti-WTMC antibody moiety that comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 101 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 102. In some embodiments, the anti-WTMC construct comprises an anti-WTMC antibody moiety that comprises a V_{H} domain comprising SEQ ID NO: 101 and a V_{L} domain comprising SEQ ID NO: 102. An anti-WTMC antibody moiety comprising SEQ ID NO: 101 and SEQ ID NO: 102 is alternatively referred to herein as a “Clone 18 anti-WTMC antibody moiety”.

In some embodiments, the anti-WTMC construct comprises an anti-WTMC antibody moiety that comprises: (a) a HC-CDR1 comprising an amino acid sequence set forth in SEQ ID NO: 55; (b) a HC-CDR2 comprising an amino acid sequence set forth in SEQ ID NO: 56; (c) a HC-CDR3 comprising an amino acid sequence set forth in SEQ ID NO: 57; (d) a LC-CDR1 comprising an amino acid sequence set forth in SEQ ID NO: 58; (e) a LC-CDR2 comprising an amino acid sequence set forth in SEQ ID NO: 59; and (f) a LC-CDR3 comprising an amino acid sequence set forth in SEQ ID NO: 60. In some embodiments, the CDRs are human CDRs. In some embodiments, the anti-WTMC construct comprises an anti-WTMC antibody moiety that comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 103 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 104. In some embodiments, the anti-WTMC construct comprises an anti-WTMC antibody moiety that comprises a V_{H} domain comprising SEQ ID NO: 103 and a V_{L} domain comprising SEQ ID NO: 104. An anti-WTMC antibody moiety comprising SEQ ID NO: 103 and SEQ ID NO: 104 is alternatively referred to herein as a “Clone 26 anti-WTMC antibody moiety”.

In some embodiments, the anti-WTMC construct comprises an anti-WTMC antibody moiety that comprises: (a) a HC-CDR1 comprising an amino acid sequence set forth in SEQ ID NO: 61; (b) a HC-CDR2 comprising an amino acid sequence set forth in SEQ ID NO: 62; (c) a HC-CDR3 comprising an amino acid sequence set forth in SEQ ID NO: 63; (d) a LC-CDR1 comprising an amino acid sequence set forth in SEQ ID NO: 64; (e) a LC-CDR2 comprising an amino acid sequence set forth in SEQ ID NO: 65; and (f) a LC-CDR3 comprising an amino acid sequence set forth in SEQ ID NO: 66. In some embodiments, the CDRs are human CDRs. In some embodiments, the anti-WTMC construct comprises an anti-WTMC antibody moiety that comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 105 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 106. In some embodiments, the anti-WTMC construct comprises an anti-WTMC antibody moiety that comprises a V_{H} domain comprising SEQ ID NO: 105 and a V_{L} domain comprising SEQ ID NO: 106. An anti-WTMC antibody moiety comprising SEQ ID NO: 105 and SEQ ID NO: 106 is alternatively referred to herein as a “Clone 30 anti-WTMC antibody moiety”.

In some embodiments, the anti-WTMC construct comprises an anti-WTMC antibody moiety that comprises: (a) a HC-CDR1 comprising an amino acid sequence set forth in SEQ ID NO: 67; (b) a HC-CDR2 comprising an amino acid sequence set forth in SEQ ID NO: 68; (c) a HC-CDR3 comprising an amino acid sequence set forth in SEQ ID NO: 69; (d) a LC-CDR1 comprising an amino acid sequence set forth in SEQ ID NO: 70; (e) a LC-CDR2 comprising an amino acid sequence set forth in SEQ ID NO: 71; and (f) a LC-CDR3 comprising an amino acid sequence set forth in SEQ ID NO: 72. In some embodiments, the CDRs are human CDRs. In some embodiments, the anti-WTMC construct comprises an anti-WTMC antibody moiety that comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 107 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 108. In some embodiments, the anti-WTMC construct comprises an anti-WTMC antibody moiety that comprises a V_{H} domain comprising SEQ ID NO: 107 and a V_{L} domain comprising SEQ ID NO: 108. An anti-WTMC antibody moiety comprising SEQ ID NO: 107 and SEQ ID NO: 108 is alternatively referred to herein as a “Clone 32 anti-WTMC antibody moiety”.

In some embodiments, the anti-WTMC construct comprises an anti-WTMC antibody moiety that comprises: (a) a HC-CDR1 comprising an amino acid sequence set forth in SEQ ID NO: 73; (b) a HC-CDR2 comprising an amino acid sequence set forth in SEQ ID NO: 74; (c) a HC-CDR3 comprising an amino acid sequence set forth in SEQ ID NO: 75; (d) a LC-CDR1 comprising an amino acid sequence set forth in SEQ ID NO: 76; (e) a LC-CDR2 comprising an amino acid sequence set forth in SEQ ID NO: 77; and (f) a LC-CDR3 comprising an amino acid sequence set forth in SEQ ID NO: 78. In some embodiments, the CDRs are human CDRs. In some embodiments, the anti-WTMC construct comprises an anti-WTMC antibody moiety that comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 109 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 110. In some embodiments, the anti-WTMC construct comprises an anti-WTMC antibody moiety that comprises a V_{H} domain comprising SEQ ID NO: 109 and a V_{L} domain comprising SEQ ID NO: 110. An anti-WTMC antibody moiety comprising SEQ ID NO: 109 and SEQ ID NO: 110 is alternatively referred to herein as a “Clone 34 anti-WTMC antibody moiety”.

In some embodiments, the anti-WTMC construct comprises an anti-WTMC antibody moiety that comprises: (a) a HC-CDR1 comprising an amino acid sequence set forth in SEQ ID NO: 79; (b) a HC-CDR2 comprising an amino acid sequence set forth in SEQ ID NO: 80; (c) a HC-CDR3 comprising an amino acid sequence set forth in SEQ ID NO: 81; (d) a LC-CDR1 comprising an amino acid sequence set forth in SEQ ID NO: 82; (e) a LC-CDR2 comprising an amino acid sequence set forth in SEQ ID NO: 83; and (f) a LC-CDR3 comprising an amino acid sequence set forth in SEQ ID NO: 84. In some embodiments, the CDRs are human CDRs. In some embodiments, the anti-WTMC construct comprises an anti-WTMC antibody moiety that comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 111 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 112. In some embodiments, the anti-WTMC construct comprises an anti-WTMC antibody moiety that comprises a V_{H} domain comprising SEQ ID NO: 111 and a V_{L} domain comprising SEQ ID NO: 112. An anti-WTMC antibody moiety comprising SEQ ID NO: 111 and SEQ ID NO: 112 is alternatively referred to herein as a “Clone 36 anti-WTMC antibody moiety”.

### Anti-WTMC Antibody Moieties

The anti-WTMC constructs comprise an anti-WTMC antibody moiety that specifically binds to a complex comprising a WT1 peptide (e.g., WT1-RMF) and an MHC class I protein.

In some embodiments, the anti-WTMC antibody moiety specifically binds to an WTMC present on the surface of a cell. In some embodiments, the cell is a cancer cell. In some embodiments, the cancer cell is in a solid tumor. In some embodiments, the cancer cell is a metastatic cancer cell.

In some embodiments, the WT1 peptide (e.g., WT1-RMF) is an MHC class I-restricted peptide. In some embodiments, the WT1 peptide (e.g., WT1-RMF) is from about 8 to about 12 (such as about any of 8, 9, 10, 11, or 12) amino acids in length. In some embodiments, the WT1 peptide (e.g., WT1-RMF) comprises (e.g., consists of) the amino acid sequence of SEQ ID NO: 113. In some embodiments, the antibody moiety specifically recognizes amino acid residues FPNA (SEQ ID NO: 349) of SEQ ID NO: 113.

In some embodiments, the MHC class I protein is HLA-A, HLA-B, HLA-C, HLA-E, HLA-F, or HLA-G. In some embodiments, the MHC class I protein is HLA-A. In some embodiments, the HLA-A is HLA-A02. In some embodiments, the HLA-A02 is HLA-A*02:01.

In some embodiments, the anti-WTMC antibody moiety is a full-length antibody. In some embodiments, the anti-WTMC antibody moiety is an antigen-binding fragment, for example an antigen-binding fragment selected from the group consisting of a Fab, a Fab′, a F(ab′)2, an Fv fragment, a disulfide stabilized Fv fragment (dsFv), and a single-chain antibody molecule (scFv). In some embodiments, the anti-WTMC antibody moiety is an scFv. In some embodiments, the anti-WTMC antibody moiety is human, humanized, or semi-synthetic.

In some embodiments, the anti-WTMC antibody moiety of the anti-WTMC construct is a full-length antibody. In some embodiments, the anti-WTMC antibody moiety of the anti-WTMC construct is an antigen-binding fragment of an anti-WTMC antibody, for example, an antigen-binding fragment selected from the group consisting of a Fab, a Fab′, a F(ab′)2, an Fv fragment, a disulfide stabilized Fv fragment (dsFv), and a single-chain antibody molecule (scFv). In some embodiments, the anti-WTMC antibody moiety of the anti-WTMC construct is an scFv. In some embodiments, the anti-WTMC antibody moiety is human, humanized, or semi-synthetic.

In some embodiments, the anti-WTMC construct comprises an anti-WTMC antibody moiety that binds a human WTMC complex, a mouse WTMC complex, a rat WTMC complex, a cynomolgus monkey WTMC complex, and/or a rhesus WTMC complex. In some embodiments, the anti-WTMC construct comprises an anti-WTMC antibody moiety that specifically binds a human WTMC complex. In some embodiments, the anti-WTMC construct comprises an anti-WTMC antibody moiety that specifically binds to a WTMC complex present on or expressed on the surface of a cell. In some embodiments, the cell is a cancer cell. In some embodiments, the cell expresses abnormally high levels of a WTMC complex, as compared to a reference cell. In some embodiments, the reference cell is a cell obtained from or derived from non-diseased (such as non-cancerous) tissue. In some embodiments, the cell that expresses abnormally high levels of a WTMC complex is a cancer cell. In some embodiments, the cancer cell is in a solid tumor. In some embodiments, the cancer cell is a WT1-positive cancer cell (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma). In some embodiments, the cancer cell is a metastatic cancer cell.

In some embodiments, the anti-WTMC antibody moiety (or the anti-WTMC construct comprising the anti-WTMC antibody moiety) binds to the complex comprising the WT1 peptide (e.g., WT1-RMF) and the MHC class I protein with an affinity that is at least about 10 (including for example at least about any of 10, 20, 30, 40, 50, 75, 100, 200, 300, 400, 500, 750, 1000 or more) times its binding affinity for each of full-length cathepsin, free WT1 peptide (e.g., WT1-RMF), MHC class I protein not bound to a peptide, and/or MHC class I protein bound to a non-WT1 peptide. In some embodiments, the anti-WTMC antibody moiety (or the anti-WTMC construct comprising the anti-WTMC antibody moiety) binds to the complex comprising the WT1 peptide (e.g., WT1-RMF) and the MHC class I protein with a K_{d} no more than about 1/10 (such as no more than about any of 1/10, 1/20, 1/30, 1/40, 1/50, 1/75, 1/100, 1/200, 1/300, 1/400, 1/500, 1/750, 1/1000 or less) times its K_{d} for binding to each of full-length cathepsin, free WT1 peptide (e.g., WT1-RMF), MHC class I protein not bound to a peptide, and/or MHC class I protein bound to a non-WT1 peptide.

In some embodiments, the anti-WTMC antibody moiety (or the anti-WTMC construct comprising the anti-WTMC antibody moiety) binds to the complex comprising the WT1 peptide (e.g., WT1-RMF) and the MHC class I protein with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the anti-WTMC antibody moiety (or the anti-WTMC construct comprising the anti-WTMC antibody moiety) binds to the complex comprising the WT1 peptide (e.g., WT1-RMF) and the MHC class I protein with a K_{d} between about 1 pM to about 250 pM (such as about any of 1, 10, 25, 50, 75, 100, 150, 200, or 250 pM, including any ranges between these values). In some embodiments, the anti-WTMC antibody moiety (or the anti-WTMC construct comprising the anti-WTMC antibody moiety) binds to the complex comprising the WT1 peptide (e.g., WT1-RMF) and the MHC class I protein with a K_{d} between about 1 nM to about 500 nM (such as about any of 1, 10, 25, 50, 75, 100, 150, 200, 250, 300, 350, 400, 450, or 500 nM, including any ranges between these values).

In some embodiments, the anti-WTMC antibody moiety specifically binds to a complex comprising a WT1 peptide (e.g., WT1-RMF) comprising the amino acid sequence of SEQ ID NO: 113 and HLA-A*02:01, wherein the anti-WTMC antibody moiety cross-reacts with at least one (including at least about any of 2, 3, 4, 5, or 6) of: a complex comprising the WT1 peptide (e.g., WT1-RMF) and HLA-A*02:02 (GenBank Accession No.: AFL91480), a complex comprising the WT1 peptide (e.g., WT1-RMF) and HLA-A*02:03 (GenBank Accession No.: AAA03604), a complex comprising the WT1 peptide (e.g., WT1-RMF) and HLA-A*02:05 (GenBank Accession No.: AAA03603), a complex comprising the WT1 peptide (e.g., WT1-RMF) and HLA-A*02:06 (GenBank Accession No.: CCB78868), a complex comprising the WT1 peptide (e.g., WT1-RMF) and HLA-A*02:07 (GenBank Accession No.: ACR55712), and a complex comprising the WT1 peptide (e.g., WT1-RMF) and HLA-A*02:11 (GenBank Accession No.: CAB56609). In some embodiments, the antibody moiety specifically recognizes amino acid residues FPNA (SEQ ID NO: 349) of SEQ ID NO: 113.

In some embodiments, the anti-WTMC antibody moiety is a semi-synthetic antibody moiety comprising fully human sequences and one or more synthetic regions. In some embodiments, the anti-WTMC antibody moiety is a semi-synthetic antibody moiety comprising a fully human light chain variable domain and a semi-synthetic heavy chain variable domain comprising fully human FR1, HC-CDR1, FR2, HC-CDR2, FR3, and FR4 regions and a synthetic HC-CDR3. In some embodiments, the semi-synthetic heavy chain variable domain comprises a fully synthetic HC-CDR3 having a sequence from about 5 to about 25 (such as about any of 5, 6, 7, 8, 9, 10, 11, 12, 13, 14, 15, 16, 17, 18, 19, 20, 21, 22, 23, 24, or 25) amino acids in length. In some embodiments, the semi-synthetic heavy chain variable domain or the synthetic HC-CDR3 is obtained from a semi-synthetic library (such as a semi-synthetic human library) comprising fully synthetic HC-CDR3s having a sequence from about 5 to about 25 (such as about any of 5, 6, 7, 8, 9, 10, 11, 12, 13, 14, 15, 16, 17, 18, 19, 20, 21, 22, 23, 24, or 25) amino acids in length, wherein each amino acid in the sequence is randomly selected from the standard human amino acids, minus cysteine. In some embodiments, the synthetic HC-CDR3 is from about 7 to about 15 (such as about any of 7, 8, 9, 10, 11, 12, 13, 14, or 15) amino acids in length.

The anti-WTMC antibody moieties in some embodiments comprise specific sequences or certain variants of such sequences. In some embodiments, the amino acid substitutions in the variant sequences do not substantially reduce the ability of the anti-WTMC antibody moiety to bind the WTMC. For example, alterations that do not substantially reduce WTMC binding affinity may be made. Alterations that substantially improve WTMC binding affinity or affect some other property, such as specificity and/or cross-reactivity with related variants of the WTMC, are also contemplated.

In some embodiments, the anti-WTMC antibody moiety specifically binds to a WTMC complex (e.g., a complex comprising a WT1 peptide (e.g., WT1-RMF) and an MHC class I protein expressed on the surface of a cell, such as a cancer cell) and competes for binding to WTMC with a second anti-WTMC antibody (or antibody moiety) comprising: (a) a heavy chain complementarity determining region (HC-CDR) 1 comprising an amino acid sequence set forth in any one of SEQ ID NOs: 1, 7, 13, 19, 25, 31, 37, 43, 49, 55, 61, 67, 73, or 79 or a variant thereof comprising up to about 3 (such as about any of 1, 2, or 3) amino acid substitutions; (b) a HC-CDR2 comprising an amino acid sequence set forth in any one of SEQ ID NOs: 2, 8, 14, 20, 26, 32, 38, 44, 50, 56, 62, 68, 74, or 80 or a variant thereof comprising up to about 3 (such as about any of 1, 2, or 3) amino acid substitutions; (c) a HC-CDR3 comprising an amino acid sequence set forth in any one of SEQ ID NOs: 3, 9, 15, 21, 27, 33, 39, 45, 51, 57, 63, 69, 75 or 81, or a variant thereof comprising up to about 3 (such as about any of 1, 2, or 3) amino acid substitutions; (d) a light chain complementarity determining region (LC-CDR) 1 comprising an amino acid sequence set forth any one of SEQ ID NOs: 4, 10, 16, 22, 28, 34, 40, 46, 52, 58, 64, 70, 76, or 82, or a variant thereof comprising up to about 3 (such as about any of 1, 2, or 3) amino acid substitutions; (e) a LC-CDR2 comprising an amino acid sequence set forth any one of SEQ ID NOs: 5, 11, 17, 23, 29, 35, 41, 47, 53, 59, 65, 71, 77, or 83, or a variant thereof comprising up to about 2 (such as about any of 1 or 2) amino acid substitutions; and (f) a LC-CDR3 comprising an amino acid sequence set forth any one of SEQ ID NOs: 6, 12, 18, 24, 30, 36, 42, 48, 54, 60, 66, 72, 78 or 84, or a variant thereof comprising up to about 3 (such as about any of 1, 2, or 3) amino acid substitutions.

In some embodiments, the anti-WTMC antibody moiety specifically binds to a WTMC complex (e.g., a complex comprising a WT1 peptide (e.g., WT1-RMF) and an MHC class I protein expressed on the surface of a cell, such as a cancer cell) and competes for binding to WTMC with a second anti-WTMC antibody (or antibody moiety) that specifically binds a WTMC complex (e.g., a complex comprising a WT1 peptide (e.g., WT1-RMF) and an MHC class I protein expressed on the surface of a cell, such as a cancer cell) and comprises: (a) a heavy chain complementarity determining region (HC-CDR) 1 comprising an amino acid sequence set forth in any one of SEQ ID NOs: 1, 7, 13, 19, 25, 31, 37, 43, 49, 55, 61, 67, 73, or 79; (b) a HC-CDR2 comprising an amino acid sequence set forth in any one of SEQ ID NOs: 2, 8, 14, 20, 26, 32, 38, 44, 50, 56, 62, 68, 74, or 80; (c) a HC-CDR3 comprising an amino acid sequence set forth in any one of SEQ ID NOs: 3, 9, 15, 21, 27, 33, 39, 45, 51, 57, 63, 69, 75 or 81; (d) a light chain complementarity determining region (LC-CDR) 1 comprising an amino acid sequence set forth any one of SEQ ID NOs: 4, 10, 16, 22, 28, 34, 40, 46, 52, 58, 64, 70, 76, or 82; (e) a LC-CDR2 comprising an amino acid sequence set forth any one of SEQ ID NOs: 5, 11, 17, 23, 29, 35, 41, 47, 53, 59, 65, 71, 77, or 83; and (f) a LC-CDR3 comprising an amino acid sequence set forth any one of SEQ ID NOs: 6, 12, 18, 24, 30, 36, 42, 48, 54, 60, 66, 72, 78 or 84.

In some embodiments, the anti-WTMC antibody moiety comprises one, two, three, four, five, or six complementarity determining region (CDR) sequences selected from the group consisting of: (a) a HC-CDR1 comprising an amino acid sequence set forth in any one of SEQ ID NOs: 1, 7, 13, 19, 25, 31, 37, 43, 49, 55, 61, 67, 73, or 79, or a variant thereof comprising up to about 3 (such as about any of 1, 2, or 3) amino acid substitutions; (b) a HC-CDR2 comprising an amino acid sequence set forth in any one of SEQ ID NOs: 2, 8, 14, 20, 26, 32, 38, 44, 50, 56, 62, 68, 74, or 80, or a variant thereof comprising up to about 3 (such as about any of 1, 2, or 3) amino acid substitutions; (c) a HC-CDR3 comprising an amino acid sequence set forth in any one of SEQ ID NOs: 3, 9, 15, 21, 27, 33, 39, 45, 51, 57, 63, 69, 75 or 81, or a variant thereof comprising up to about 3 (such as about any of 1, 2, or 3) amino acid substitutions; (d) a LC-CDR1 comprising an amino acid sequence set forth in any one of SEQ ID NOs: 4, 10, 16, 22, 28, 34, 40, 46, 52, 58, 64, 70, 76, or 82, or a variant thereof comprising up to about 3 (such as about any of 1, 2, or 3) amino acid substitutions; (e) a LC-CDR2 comprising an amino acid sequence set forth in any one of SEQ ID NOs: 5, 11, 17, 23, 29, 35, 41, 47, 53, 59, 65, 71, 77, or 83, or a variant thereof comprising up to about 2 (such as about any of 1 or 2) amino acid substitutions; and (f) a LC-CDR3 comprising an amino acid sequence set forth in any one of SEQ ID NOs: 6, 12, 18, 24, 30, 36, 42, 48, 54, 60, 66, 72, 78 or 84, or a variant thereof comprising up to about 3 (such as about any of 1, 2, or 3) amino acid substitutions.

In some embodiments, the anti-WTMC construct comprises an anti-WTMC antibody moiety that comprises (a) a HC-CDR1 comprising an amino acid sequence set forth in any one of SEQ ID NOs: 1, 7, 13, 19, 25, 31, 37, 43, 49, 55, 61, 67, 73, or 79, or a variant thereof comprising up to about 3 (such as about any of 1, 2, or 3) amino acid substitutions; (b) a HC-CDR2 comprising an amino acid sequence set forth in any one of SEQ ID NOs: 2, 8, 14, 20, 26, 32, 38, 44, 50, 56, 62, 68, 74, or 80, or a variant thereof comprising up to about 3 (such as about any of 1, 2, or 3) amino acid substitutions; (c) a HC-CDR3 comprising an amino acid sequence set forth in any one of SEQ ID NOs: 3, 9, 15, 21, 27, 33, 39, 45, 51, 57, 63, 69, 75 or 81, or a variant thereof comprising up to about 3 (such as about any of 1, 2, or 3) amino acid substitutions; (d) a LC-CDR1 comprising an amino acid sequence set forth in any one of SEQ ID NOs: 4, 10, 16, 22, 28, 34, 40, 46, 52, 58, 64, 70, 76, or 82, or a variant thereof comprising up to about 3 (such as about any of 1, 2, or 3) amino acid substitutions; (e) a LC-CDR2 comprising an amino acid sequence set forth in any one of SEQ ID NOs: 5, 11, 17, 23, 29, 35, 41, 47, 53, 59, 65, 71, 77, or 83, or a variant thereof comprising up to about 2 (such as about any of 1 or 2) amino acid substitutions; and (f) a LC-CDR3 comprising an amino acid sequence set forth in any one of SEQ ID NOs: 6, 12, 18, 24, 30, 36, 42, 48, 54, 60, 66, 72, 78 or 84, or a variant thereof comprising up to about 3 (such as about any of 1, 2, or 3) amino acid substitutions.

In some embodiments, the anti-WTMC antibody moiety comprises one, two, three, four, five, or six complementarity determining region (CDR) sequences selected from the group consisting of: (a) a HC-CDR1 comprising an amino acid sequence set forth in any one of SEQ ID NOs: 1, 7, 13, 19, 25, 31, 37, 43, 49, 55, 61, 67, 73, or 79; (b) a HC-CDR2 comprising an amino acid sequence set forth in any one of SEQ ID NOs: 2, 8, 14, 20, 26, 32, 38, 44, 50, 56, 62, 68, 74, or 80; (c) a HC-CDR3 comprising an amino acid sequence set forth in any one of SEQ ID NOs: 3, 9, 15, 21, 27, 33, 39, 45, 51, 57, 63, 69, 75 or 81; (d) a LC-CDR1 comprising an amino acid sequence set forth in any one of SEQ ID NOs: 4, 10, 16, 22, 28, 34, 40, 46, 52, 58, 64, 70, 76, or 82; (e) a LC-CDR2 comprising an amino acid sequence set forth in any one of SEQ ID NOs: 5, 11, 17, 23, 29, 35, 41, 47, 53, 59, 65, 71, 77, or 83; and (f) a LC-CDR3 comprising an amino acid sequence set forth in any one of SEQ ID NOs: 6, 12, 18, 24, 30, 36, 42, 48, 54, 60, 66, 72, 78 or 84.

In some embodiments, the anti-WTMC antibody moiety comprises: (a) a HC-CDR1 comprising an amino acid sequence set forth in any one of SEQ ID NOs: 1, 7, 13, 19, 25, 31, 37, 43, 49, 55, 61, 67, 73, or 79; (b) a HC-CDR2 comprising an amino acid sequence set forth in any one of SEQ ID NOs: 2, 8, 14, 20, 26, 32, 38, 44, 50, 56, 62, 68, 74, or 80; (c) a HC-CDR3 comprising an amino acid sequence set forth in any one of SEQ ID NOs: 3, 9, 15, 21, 27, 33, 39, 45, 51, 57, 63, 69, 75 or 81; (d) a LC-CDR1 comprising an amino acid sequence set forth in any one of SEQ ID NOs: 4, 10, 16, 22, 28, 34, 40, 46, 52, 58, 64, 70, 76, or 82; (e) a LC-CDR2 comprising an amino acid sequence set forth in any one of SEQ ID NOs: 5, 11, 17, 23, 29, 35, 41, 47, 53, 59, 65, 71, 77, or 83; and (f) a LC-CDR3 comprising an amino acid sequence set forth in any one of SEQ ID NOs: 6, 12, 18, 24, 30, 36, 42, 48, 54, 60, 66, 72, 78 or 84.

In some embodiments, the anti-WTMC antibody moiety comprises one, two, three, four five, or six CDRs of antibody clone 1 as shown in Tables B1-B2. In some embodiments, the antibody comprises the VH and/or the VL of antibody clone 1 as shown in Table D.

In some embodiments, the anti-WTMC antibody moiety comprises: (a) a HC-CDR1 comprising an amino acid sequence set forth in SEQ ID NO: 1, or a variant thereof comprising up to about 3 (such as about any of 1, 2, or 3) amino acid substitutions; (b) a HC-CDR2 comprising an amino acid sequence set forth in SEQ ID NO: 2, or a variant thereof comprising up to about 3 (such as about any of 1, 2, or 3) amino acid substitutions; (c) a HC-CDR3 comprising an amino acid sequence set forth in SEQ ID NO: 3, or a variant thereof comprising up to about 3 (such as about any of 1, 2, or 3) amino acid substitutions; (d) a LC-CDR1 comprising an amino acid sequence set forth in SEQ ID NO: 4, or a variant thereof comprising up to about 3 (such as about any of 1, 2, or 3) amino acid substitutions; (e) a LC-CDR2 comprising an amino acid sequence set forth in SEQ ID NO: 5, or a variant thereof comprising up to about 2 (such as about any of 1 or 2) amino acid substitutions; and (f) a LC-CDR3 comprising an amino acid sequence set forth in SEQ ID NO: 6, or a variant thereof comprising up to about 3 (such as about any of 1, 2, or 3) amino acid substitutions.

In some embodiments, the anti-WTMC antibody moiety comprises: (a) a HC-CDR1 comprising an amino acid sequence set forth in SEQ ID NO: 1; (b) a HC-CDR2 comprising an amino acid sequence set forth in SEQ ID NO: 2; (c) a HC-CDR3 comprising an amino acid sequence set forth in SEQ ID NO: 3; (d) a LC-CDR1 comprising an amino acid sequence set forth in SEQ ID NO: 4; (e) a LC-CDR2 comprising an amino acid sequence set forth in SEQ ID NO: 5; and (f) a LC-CDR3 comprising an amino acid sequence set forth in SEQ ID NO: 6.

In some embodiments, the anti-WTMC antibody moiety comprises a V_{H} comprising the amino acid sequences set forth in SEQ ID NOs: 1-3, and a V_{L} comprising the amino acid sequences set forth in SEQ ID NOs: 4-6.

In some embodiments, the anti-WTMC antibody moiety comprises one, two, three, four five, or six CDRs of antibody clone 2 as shown in Tables B & C. In some embodiments, the antibody comprises the V_{H} and/or the V_{L} of antibody clone 2 as shown in Table 4.

In some embodiments, the anti-WTMC antibody moiety comprises: (a) a HC-CDR1 comprising an amino acid sequence set forth in SEQ ID NO: 7, or a variant thereof comprising up to about 3 (such as about any of 1, 2, or 3) amino acid substitutions; (b) a HC-CDR2 comprising an amino acid sequence set forth in SEQ ID NO: 8, or a variant thereof comprising up to about 3 (such as about any of 1, 2, or 3) amino acid substitutions; (c) a HC-CDR3 comprising an amino acid sequence set forth in SEQ ID NO: 9, or a variant thereof comprising up to about 3 (such as about any of 1, 2, or 3) amino acid substitutions; (d) a LC-CDR1 comprising an amino acid sequence set forth in SEQ ID NO: 10, or a variant thereof comprising up to about 3 (such as about any of 1, 2, or 3) amino acid substitutions; (e) a LC-CDR2 comprising an amino acid sequence set forth in SEQ ID NO: 11, or a variant thereof comprising up to about 2 (such as about any of 1 or 2) amino acid substitutions; and (f) a LC-CDR3 comprising an amino acid sequence set forth in SEQ ID NO: 12, or a variant thereof comprising up to about 3 (such as about any of 1, 2, or 3) amino acid substitutions.

In some embodiments, the anti-WTMC antibody moiety comprises: (a) a HC-CDR1 comprising an amino acid sequence set forth in SEQ ID NO: 7; (b) a HC-CDR2 comprising an amino acid sequence set forth in SEQ ID NO: 8; (c) a HC-CDR3 comprising an amino acid sequence set forth in SEQ ID NO: 9; (d) a LC-CDR1 comprising an amino acid sequence set forth in SEQ ID NO: 10; (e) a LC-CDR2 comprising an amino acid sequence set forth in SEQ ID NO: 11; and (f) a LC-CDR3 comprising an amino acid sequence set forth in SEQ ID NO: 12.

In some embodiments, the anti-WTMC antibody moiety comprises a VH comprising the amino acid sequences set forth in SEQ ID NOs: 7-9, and a VL comprising the amino acid sequences set forth in SEQ ID NOs: 10-12.

In some embodiments, the anti-WTMC antibody moiety comprises one, two, three, four five, or six CDRs of antibody clone 10 as shown in Tables B & C. In some embodiments, the antibody comprises the VH and/or the VL of antibody clone 10 as shown in Table 4.

In some embodiments, the anti-WTMC antibody moiety comprises: (a) a HC-CDR1 comprising an amino acid sequence set forth in SEQ ID NO: 13, or a variant thereof comprising up to about 3 (such as about any of 1, 2, or 3) amino acid substitutions; (b) a HC-CDR2 comprising an amino acid sequence set forth in SEQ ID NO: 14, or a variant thereof comprising up to about 3 (such as about any of 1, 2, or 3) amino acid substitutions; (c) a HC-CDR3 comprising an amino acid sequence set forth in SEQ ID NO: 15, or a variant thereof comprising up to about 3 (such as about any of 1, 2, or 3) amino acid substitutions; (d) a LC-CDR1 comprising an amino acid sequence set forth in SEQ ID NO: 16, or a variant thereof comprising up to about 3 (such as about any of 1, 2, or 3) amino acid substitutions; (e) a LC-CDR2 comprising an amino acid sequence set forth in SEQ ID NO: 17, or a variant thereof comprising up to about 2 (such as about any of 1 or 2) amino acid substitutions; and (f) a LC-CDR3 comprising an amino acid sequence set forth in SEQ ID NO: 18, or a variant thereof comprising up to about 3 (such as about any of 1, 2, or 3) amino acid substitutions.

In some embodiments, the anti-WTMC antibody moiety comprises: (a) a HC-CDR1 comprising an amino acid sequence set forth in SEQ ID NO: 13; (b) a HC-CDR2 comprising an amino acid sequence set forth in SEQ ID NO: 14; (c) a HC-CDR3 comprising an amino acid sequence set forth in SEQ ID NO: 15; (d) a LC-CDR1 comprising an amino acid sequence set forth in SEQ ID NO: 16; (e) a LC-CDR2 comprising an amino acid sequence set forth in SEQ ID NO: 17; and (f) a LC-CDR3 comprising an amino acid sequence set forth in SEQ ID NO: 18.

In some embodiments, the anti-WTMC antibody moiety comprises a VH comprising the amino acid sequences set forth in SEQ ID NOs: 13-15, and a VL comprising the amino acid sequences set forth in SEQ ID NOs: 16-18.

In some embodiments, the anti-WTMC antibody moiety comprises one, two, three, four five, or six CDRs of antibody clone 11 as shown in Tables B & C. In some embodiments, the antibody comprises the VH and/or the VL of antibody clone 11 as shown in Table 4.

In some embodiments, the anti-WTMC antibody moiety comprises: (a) a HC-CDR1 comprising an amino acid sequence set forth in SEQ ID NO: 19, or a variant thereof comprising up to about 3 (such as about any of 1, 2, or 3) amino acid substitutions; (b) a HC-CDR2 comprising an amino acid sequence set forth in SEQ ID NO: 20, or a variant thereof comprising up to about 3 (such as about any of 1, 2, or 3) amino acid substitutions; (c) a HC-CDR3 comprising an amino acid sequence set forth in SEQ ID NO: 21, or a variant thereof comprising up to about 3 (such as about any of 1, 2, or 3) amino acid substitutions; (d) a LC-CDR1 comprising an amino acid sequence set forth in SEQ ID NO: 22, or a variant thereof comprising up to about 3 (such as about any of 1, 2, or 3) amino acid substitutions; (e) a LC-CDR2 comprising an amino acid sequence set forth in SEQ ID NO: 23, or a variant thereof comprising up to about 2 (such as about any of 1 or 2) amino acid substitutions; and (f) a LC-CDR3 comprising an amino acid sequence set forth in SEQ ID NO: 24, or a variant thereof comprising up to about 3 (such as about any of 1, 2, or 3) amino acid substitutions.

In some embodiments, the anti-WTMC antibody moiety comprises: (a) a HC-CDR1 comprising an amino acid sequence set forth in SEQ ID NO: 19; (b) a HC-CDR2 comprising an amino acid sequence set forth in SEQ ID NO: 20; (c) a HC-CDR3 comprising an amino acid sequence set forth in SEQ ID NO: 21; (d) a LC-CDR1 comprising an amino acid sequence set forth in SEQ ID NO: 22; (e) a LC-CDR2 comprising an amino acid sequence set forth in SEQ ID NO: 23; and (f) a LC-CDR3 comprising an amino acid sequence set forth in SEQ ID NO: 24.

In some embodiments, the anti-WTMC antibody moiety comprises a VH comprising the amino acid sequences set forth in SEQ ID NOs: 19-21, and a VL comprising the amino acid sequences set forth in SEQ ID NOs: 22-24.

In some embodiments, the anti-WTMC antibody moiety comprises one, two, three, four five, or six CDRs of antibody clone 12 as shown in Tables B & C. In some embodiments, the antibody comprises the VH and/or the VL of antibody clone 12 as shown in Table 4.

In some embodiments, the anti-WTMC antibody moiety comprises: (a) a HC-CDR1 comprising an amino acid sequence set forth in SEQ ID NO: 25, or a variant thereof comprising up to about 3 (such as about any of 1, 2, or 3) amino acid substitutions; (b) a HC-CDR2 comprising an amino acid sequence set forth in SEQ ID NO: 26, or a variant thereof comprising up to about 3 (such as about any of 1, 2, or 3) amino acid substitutions; (c) a HC-CDR3 comprising an amino acid sequence set forth in SEQ ID NO: 27, or a variant thereof comprising up to about 3 (such as about any of 1, 2, or 3) amino acid substitutions; (d) a LC-CDR1 comprising an amino acid sequence set forth in SEQ ID NO: 28, or a variant thereof comprising up to about 3 (such as about any of 1, 2, or 3) amino acid substitutions; (e) a LC-CDR2 comprising an amino acid sequence set forth in SEQ ID NO: 29, or a variant thereof comprising up to about 2 (such as about any of 1 or 2) amino acid substitutions; and (f) a LC-CDR3 comprising an amino acid sequence set forth in SEQ ID NO: 30, or a variant thereof comprising up to about 3 (such as about any of 1, 2, or 3) amino acid substitutions.

In some embodiments, the anti-WTMC antibody moiety comprises: (a) a HC-CDR1 comprising an amino acid sequence set forth in SEQ ID NO: 25; (b) a HC-CDR2 comprising an amino acid sequence set forth in SEQ ID NO: 26; (c) a HC-CDR3 comprising an amino acid sequence set forth in SEQ ID NO: 27; (d) a LC-CDR1 comprising an amino acid sequence set forth in SEQ ID NO: 28; (e) a LC-CDR2 comprising an amino acid sequence set forth in SEQ ID NO: 29; and (f) a LC-CDR3 comprising an amino acid sequence set forth in SEQ ID NO: 30.

In some embodiments, the anti-WTMC antibody moiety comprises a VH comprising the amino acid sequences set forth in SEQ ID NOs: 25-27, and a VL comprising the amino acid sequences set forth in SEQ ID NOs: 28-30.

In some embodiments, the anti-WTMC antibody moiety comprises one, two, three, four five, or six CDRs of antibody clone 14 as shown in Tables B & C. In some embodiments, the antibody comprises the VH and/or the VL of antibody clone 14 as shown in Table 4.

In some embodiments, the anti-WTMC antibody moiety comprises: (a) a HC-CDR1 comprising an amino acid sequence set forth in SEQ ID NO: 31, or a variant thereof comprising up to about 3 (such as about any of 1, 2, or 3) amino acid substitutions; (b) a HC-CDR2 comprising an amino acid sequence set forth in SEQ ID NO: 32, or a variant thereof comprising up to about 3 (such as about any of 1, 2, or 3) amino acid substitutions; (c) a HC-CDR3 comprising an amino acid sequence set forth in SEQ ID NO: 33, or a variant thereof comprising up to about 3 (such as about any of 1, 2, or 3) amino acid substitutions; (d) a LC-CDR1 comprising an amino acid sequence set forth in SEQ ID NO: 34, or a variant thereof comprising up to about 3 (such as about any of 1, 2, or 3) amino acid substitutions; (e) a LC-CDR2 comprising an amino acid sequence set forth in SEQ ID NO: 35, or a variant thereof comprising up to about 2 (such as about any of 1 or 2) amino acid substitutions; and (f) a LC-CDR3 comprising an amino acid sequence set forth in SEQ ID NO: 36, or a variant thereof comprising up to about 3 (such as about any of 1, 2, or 3) amino acid substitutions.

In some embodiments, the anti-WTMC antibody moiety comprises: (a) a HC-CDR1 comprising an amino acid sequence set forth in SEQ ID NO: 31; (b) a HC-CDR2 comprising an amino acid sequence set forth in SEQ ID NO: 32; (c) a HC-CDR3 comprising an amino acid sequence set forth in SEQ ID NO: 33; (d) a LC-CDR1 comprising an amino acid sequence set forth in SEQ ID NO: 34; (e) a LC-CDR2 comprising an amino acid sequence set forth in SEQ ID NO: 35; and (f) a LC-CDR3 comprising an amino acid sequence set forth in SEQ ID NO: 36.

In some embodiments, the anti-WTMC antibody moiety comprises a VH comprising the amino acid sequences set forth in SEQ ID NOs: 31-33, and a VL comprising the amino acid sequences set forth in SEQ ID NOs: 34-36.

In some embodiments, the anti-WTMC antibody moiety comprises one, two, three, four five, or six CDRs of antibody clone 15 as shown in Tables B & C. In some embodiments, the antibody comprises the VH and/or the VL of antibody clone 15 as shown in Table 4.

In some embodiments, the anti-WTMC antibody moiety comprises: (a) a HC-CDR1 comprising an amino acid sequence set forth in SEQ ID NO: 37, or a variant thereof comprising up to about 3 (such as about any of 1, 2, or 3) amino acid substitutions; (b) a HC-CDR2 comprising an amino acid sequence set forth in SEQ ID NO: 38, or a variant thereof comprising up to about 3 (such as about any of 1, 2, or 3) amino acid substitutions; (c) a HC-CDR3 comprising an amino acid sequence set forth in SEQ ID NO: 39, or a variant thereof comprising up to about 3 (such as about any of 1, 2, or 3) amino acid substitutions; (d) a LC-CDR1 comprising an amino acid sequence set forth in SEQ ID NO: 40, or a variant thereof comprising up to about 3 (such as about any of 1, 2, or 3) amino acid substitutions; (e) a LC-CDR2 comprising an amino acid sequence set forth in SEQ ID NO: 41, or a variant thereof comprising up to about 2 (such as about any of 1 or 2) amino acid substitutions; and (f) a LC-CDR3 comprising an amino acid sequence set forth in SEQ ID NO: 42, or a variant thereof comprising up to about 3 (such as about any of 1, 2, or 3) amino acid substitutions.

In some embodiments, the anti-WTMC antibody moiety comprises: (a) a HC-CDR1 comprising an amino acid sequence set forth in SEQ ID NO: 37; (b) a HC-CDR2 comprising an amino acid sequence set forth in SEQ ID NO: 38; (c) a HC-CDR3 comprising an amino acid sequence set forth in SEQ ID NO: 39; (d) a LC-CDR1 comprising an amino acid sequence set forth in SEQ ID NO: 40; (e) a LC-CDR2 comprising an amino acid sequence set forth in SEQ ID NO: 41; and (f) a LC-CDR3 comprising an amino acid sequence set forth in SEQ ID NO: 42.

In some embodiments, the anti-WTMC antibody moiety comprises a VH comprising the amino acid sequences set forth in SEQ ID NOs: 37-39, and a VL comprising the amino acid sequences set forth in SEQ ID NOs: 40-42.

In some embodiments, the anti-WTMC antibody moiety comprises one, two, three, four five, or six CDRs of antibody clone 17 as shown in Tables B & C. In some embodiments, the antibody comprises the VH and/or the VL of antibody clone 17 as shown in Table 4.

In some embodiments, the anti-WTMC antibody moiety comprises: (a) a HC-CDR1 comprising an amino acid sequence set forth in SEQ ID NO: 43, or a variant thereof comprising up to about 3 (such as about any of 1, 2, or 3) amino acid substitutions; (b) a HC-CDR2 comprising an amino acid sequence set forth in SEQ ID NO: 44, or a variant thereof comprising up to about 3 (such as about any of 1, 2, or 3) amino acid substitutions; (c) a HC-CDR3 comprising an amino acid sequence set forth in SEQ ID NO: 45, or a variant thereof comprising up to about 3 (such as about any of 1, 2, or 3) amino acid substitutions; (d) a LC-CDR1 comprising an amino acid sequence set forth in SEQ ID NO: 46, or a variant thereof comprising up to about 3 (such as about any of 1, 2, or 3) amino acid substitutions; (e) a LC-CDR2 comprising an amino acid sequence set forth in SEQ ID NO: 47, or a variant thereof comprising up to about 2 (such as about any of 1 or 2) amino acid substitutions; and (f) a LC-CDR3 comprising an amino acid sequence set forth in SEQ ID NO: 48, or a variant thereof comprising up to about 3 (such as about any of 1, 2, or 3) amino acid substitutions.

In some embodiments, the anti-WTMC antibody moiety comprises: (a) a HC-CDR1 comprising an amino acid sequence set forth in SEQ ID NO: 43; (b) a HC-CDR2 comprising an amino acid sequence set forth in SEQ ID NO: 44; (c) a HC-CDR3 comprising an amino acid sequence set forth in SEQ ID NO: 45; (d) a LC-CDR1 comprising an amino acid sequence set forth in SEQ ID NO: 46; (e) a LC-CDR2 comprising an amino acid sequence set forth in SEQ ID NO: 47; and (f) a LC-CDR3 comprising an amino acid sequence set forth in SEQ ID NO: 48.

In some embodiments, the anti-WTMC antibody moiety comprises a VH comprising the amino acid sequences set forth in SEQ ID NOs: 43-45, and a VL comprising the amino acid sequences set forth in SEQ ID NOs: 46-48.

In some embodiments, the anti-WTMC antibody moiety comprises one, two, three, four five, or six CDRs of antibody clone 18 as shown in Tables B & C. In some embodiments, the antibody comprises the VH and/or the VL of antibody clone 18 as shown in Table 4.

In some embodiments, the anti-WTMC antibody moiety comprises: (a) a HC-CDR1 comprising an amino acid sequence set forth in SEQ ID NO: 49, or a variant thereof comprising up to about 3 (such as about any of 1, 2, or 3) amino acid substitutions; (b) a HC-CDR2 comprising an amino acid sequence set forth in SEQ ID NO: 50, or a variant thereof comprising up to about 3 (such as about any of 1, 2, or 3) amino acid substitutions; (c) a HC-CDR3 comprising an amino acid sequence set forth in SEQ ID NO: 51, or a variant thereof comprising up to about 3 (such as about any of 1, 2, or 3) amino acid substitutions; (d) a LC-CDR1 comprising an amino acid sequence set forth in SEQ ID NO: 52, or a variant thereof comprising up to about 3 (such as about any of 1, 2, or 3) amino acid substitutions; (e) a LC-CDR2 comprising an amino acid sequence set forth in SEQ ID NO: 53, or a variant thereof comprising up to about 2 (such as about any of 1 or 2) amino acid substitutions; and (f) a LC-CDR3 comprising an amino acid sequence set forth in SEQ ID NO: 54, or a variant thereof comprising up to about 3 (such as about any of 1, 2, or 3) amino acid substitutions.

In some embodiments, the anti-WTMC antibody moiety comprises: (a) a HC-CDR1 comprising an amino acid sequence set forth in SEQ ID NO: 49; (b) a HC-CDR2 comprising an amino acid sequence set forth in SEQ ID NO: 50; (c) a HC-CDR3 comprising an amino acid sequence set forth in SEQ ID NO: 51; (d) a LC-CDR1 comprising an amino acid sequence set forth in SEQ ID NO: 52; (e) a LC-CDR2 comprising an amino acid sequence set forth in SEQ ID NO: 53; and (f) a LC-CDR3 comprising an amino acid sequence set forth in SEQ ID NO: 54.

In some embodiments, the anti-WTMC antibody moiety comprises a VH comprising the amino acid sequences set forth in SEQ ID NOs: 49-51, and a VL comprising the amino acid sequences set forth in SEQ ID NOs: 52-54.

In some embodiments, the anti-WTMC antibody moiety comprises one, two, three, four five, or six CDRs of antibody clone 26 as shown in Tables B & C. In some embodiments, the antibody comprises the VH and/or the VL of antibody clone 26 as shown in Table 4.

In some embodiments, the anti-WTMC antibody moiety comprises: (a) a HC-CDR1 comprising an amino acid sequence set forth in SEQ ID NO: 55, or a variant thereof comprising up to about 3 (such as about any of 1, 2, or 3) amino acid substitutions; (b) a HC-CDR2 comprising an amino acid sequence set forth in SEQ ID NO: 56, or a variant thereof comprising up to about 3 (such as about any of 1, 2, or 3) amino acid substitutions; (c) a HC-CDR3 comprising an amino acid sequence set forth in SEQ ID NO: 57, or a variant thereof comprising up to about 3 (such as about any of 1, 2, or 3) amino acid substitutions; (d) a LC-CDR1 comprising an amino acid sequence set forth in SEQ ID NO: 58, or a variant thereof comprising up to about 3 (such as about any of 1, 2, or 3) amino acid substitutions; (e) a LC-CDR2 comprising an amino acid sequence set forth in SEQ ID NO: 59, or a variant thereof comprising up to about 2 (such as about any of 1 or 2) amino acid substitutions; and (f) a LC-CDR3 comprising an amino acid sequence set forth in SEQ ID NO: 60, or a variant thereof comprising up to about 3 (such as about any of 1, 2, or 3) amino acid substitutions.

In some embodiments, the anti-WTMC antibody moiety comprises: (a) a HC-CDR1 comprising an amino acid sequence set forth in SEQ ID NO: 55; (b) a HC-CDR2 comprising an amino acid sequence set forth in SEQ ID NO: 56; (c) a HC-CDR3 comprising an amino acid sequence set forth in SEQ ID NO: 57; (d) a LC-CDR1 comprising an amino acid sequence set forth in SEQ ID NO: 58; (e) a LC-CDR2 comprising an amino acid sequence set forth in SEQ ID NO: 59; and (f) a LC-CDR3 comprising an amino acid sequence set forth in SEQ ID NO: 60.

In some embodiments, the anti-WTMC antibody moiety comprises a VH comprising the amino acid sequences set forth in SEQ ID NOs: 55-57, and a VL comprising the amino acid sequences set forth in SEQ ID NOs: 58-60.

In some embodiments, the anti-WTMC antibody moiety comprises one, two, three, four five, or six CDRs of antibody clone 30 as shown in Tables B & C. In some embodiments, the antibody comprises the VH and/or the VL of antibody clone 30 as shown in Table 4.

In some embodiments, the anti-WTMC antibody moiety comprises: (a) a HC-CDR1 comprising an amino acid sequence set forth in SEQ ID NO: 61, or a variant thereof comprising up to about 3 (such as about any of 1, 2, or 3) amino acid substitutions; (b) a HC-CDR2 comprising an amino acid sequence set forth in SEQ ID NO: 62, or a variant thereof comprising up to about 3 (such as about any of 1, 2, or 3) amino acid substitutions; (c) a HC-CDR3 comprising an amino acid sequence set forth in SEQ ID NO: 63, or a variant thereof comprising up to about 3 (such as about any of 1, 2, or 3) amino acid substitutions; (d) a LC-CDR1 comprising an amino acid sequence set forth in SEQ ID NO: 64, or a variant thereof comprising up to about 3 (such as about any of 1, 2, or 3) amino acid substitutions; (e) a LC-CDR2 comprising an amino acid sequence set forth in SEQ ID NO: 65, or a variant thereof comprising up to about 2 (such as about any of 1 or 2) amino acid substitutions; and (f) a LC-CDR3 comprising an amino acid sequence set forth in SEQ ID NO: 66, or a variant thereof comprising up to about 3 (such as about any of 1, 2, or 3) amino acid substitutions.

In some embodiments, the anti-WTMC antibody moiety comprises: (a) a HC-CDR1 comprising an amino acid sequence set forth in SEQ ID NO: 61; (b) a HC-CDR2 comprising an amino acid sequence set forth in SEQ ID NO: 62; (c) a HC-CDR3 comprising an amino acid sequence set forth in SEQ ID NO: 63; (d) a LC-CDR1 comprising an amino acid sequence set forth in SEQ ID NO: 64; (e) a LC-CDR2 comprising an amino acid sequence set forth in SEQ ID NO: 65; and (f) a LC-CDR3 comprising an amino acid sequence set forth in SEQ ID NO: 66.

In some embodiments, the anti-WTMC antibody moiety comprises a VH comprising the amino acid sequences set forth in SEQ ID NOs: 61-63, and a VL comprising the amino acid sequences set forth in SEQ ID NOs: 64-66.

In some embodiments, the anti-WTMC antibody moiety comprises one, two, three, four five, or six CDRs of antibody clone 32 as shown in Tables B & C. In some embodiments, the antibody comprises the VH and/or the VL of antibody clone 32 as shown in Table 4.

In some embodiments, the anti-WTMC antibody moiety comprises: (a) a HC-CDR1 comprising an amino acid sequence set forth in SEQ ID NO: 67, or a variant thereof comprising up to about 3 (such as about any of 1, 2, or 3) amino acid substitutions; (b) a HC-CDR2 comprising an amino acid sequence set forth in SEQ ID NO: 68, or a variant thereof comprising up to about 3 (such as about any of 1, 2, or 3) amino acid substitutions; (c) a HC-CDR3 comprising an amino acid sequence set forth in SEQ ID NO: 69, or a variant thereof comprising up to about 3 (such as about any of 1, 2, or 3) amino acid substitutions; (d) a LC-CDR1 comprising an amino acid sequence set forth in SEQ ID NO: 70, or a variant thereof comprising up to about 3 (such as about any of 1, 2, or 3) amino acid substitutions; (e) a LC-CDR2 comprising an amino acid sequence set forth in SEQ ID NO: 71, or a variant thereof comprising up to about 2 (such as about any of 1 or 2) amino acid substitutions; and (f) a LC-CDR3 comprising an amino acid sequence set forth in SEQ ID NO: 72, or a variant thereof comprising up to about 3 (such as about any of 1, 2, or 3) amino acid substitutions.

In some embodiments, the anti-WTMC antibody moiety comprises: (a) a HC-CDR1 comprising an amino acid sequence set forth in SEQ ID NO: 67; (b) a HC-CDR2 comprising an amino acid sequence set forth in SEQ ID NO: 68; (c) a HC-CDR3 comprising an amino acid sequence set forth in SEQ ID NO: 69; (d) a LC-CDR1 comprising an amino acid sequence set forth in SEQ ID NO: 70; (e) a LC-CDR2 comprising an amino acid sequence set forth in SEQ ID NO: 71; and (f) a LC-CDR3 comprising an amino acid sequence set forth in SEQ ID NO: 72.

In some embodiments, the anti-WTMC antibody moiety comprises a V_{H} comprising the amino acid sequences set forth in SEQ ID NOs: 67-69, and a V_{L} comprising the amino acid sequences set forth in SEQ ID NOs: 70-72.

In some embodiments, the anti-WTMC antibody moiety comprises one, two, three, four five, or six CDRs of antibody clone 34 as shown in Tables B & C. In some embodiments, the antibody comprises the VH and/or the VL of antibody clone 34 as shown in Table 4.

In some embodiments, the anti-WTMC antibody moiety comprises: (a) a HC-CDR1 comprising an amino acid sequence set forth in SEQ ID NO: 73, or a variant thereof comprising up to about 3 (such as about any of 1, 2, or 3) amino acid substitutions; (b) a HC-CDR2 comprising an amino acid sequence set forth in SEQ ID NO: 74, or a variant thereof comprising up to about 3 (such as about any of 1, 2, or 3) amino acid substitutions; (c) a HC-CDR3 comprising an amino acid sequence set forth in SEQ ID NO: 75, or a variant thereof comprising up to about 3 (such as about any of 1, 2, or 3) amino acid substitutions; (d) a LC-CDR1 comprising an amino acid sequence set forth in SEQ ID NO: 76, or a variant thereof comprising up to about 3 (such as about any of 1, 2, or 3) amino acid substitutions; (e) a LC-CDR2 comprising an amino acid sequence set forth in SEQ ID NO: 77, or a variant thereof comprising up to about 2 (such as about any of 1 or 2) amino acid substitutions; and (f) a LC-CDR3 comprising an amino acid sequence set forth in SEQ ID NO: 78, or a variant thereof comprising up to about 3 (such as about any of 1, 2, or 3) amino acid substitutions.

In some embodiments, the anti-WTMC antibody moiety comprises: (a) a HC-CDR1 comprising an amino acid sequence set forth in SEQ ID NO: 73; (b) a HC-CDR2 comprising an amino acid sequence set forth in SEQ ID NO: 74; (c) a HC-CDR3 comprising an amino acid sequence set forth in SEQ ID NO: 75; (d) a LC-CDR1 comprising an amino acid sequence set forth in SEQ ID NO: 76; (e) a LC-CDR2 comprising an amino acid sequence set forth in SEQ ID NO: 77; and (f) a LC-CDR3 comprising an amino acid sequence set forth in SEQ ID NO: 78.

In some embodiments, the anti-WTMC antibody moiety comprises a VH comprising the amino acid sequences set forth in SEQ ID NOs: 73-75, and a VL comprising the amino acid sequences set forth in SEQ ID NOs: 76-78.

In some embodiments, the anti-WTMC antibody moiety comprises one, two, three, four five, or six CDRs of antibody clone 36 as shown in Tables B & C. In some embodiments, the antibody comprises the VH and/or the VL of antibody clone 36 as shown in Table 4.

In some embodiments, the anti-WTMC antibody moiety comprises: (a) a HC-CDR1 comprising an amino acid sequence set forth in SEQ ID NO: 79, or a variant thereof comprising up to about 3 (such as about any of 1, 2, or 3) amino acid substitutions; (b) a HC-CDR2 comprising an amino acid sequence set forth in SEQ ID NO: 80, or a variant thereof comprising up to about 3 (such as about any of 1, 2, or 3) amino acid substitutions; (c) a HC-CDR3 comprising an amino acid sequence set forth in SEQ ID NO: 81, or a variant thereof comprising up to about 3 (such as about any of 1, 2, or 3) amino acid substitutions; (d) a LC-CDR1 comprising an amino acid sequence set forth in SEQ ID NO: 82, or a variant thereof comprising up to about 3 (such as about any of 1, 2, or 3) amino acid substitutions; (e) a LC-CDR2 comprising an amino acid sequence set forth in SEQ ID NO: 83, or a variant thereof comprising up to about 2 (such as about any of 1 or 2) amino acid substitutions; and (f) a LC-CDR3 comprising an amino acid sequence set forth in SEQ ID NO: 84, or a variant thereof comprising up to about 3 (such as about any of 1, 2, or 3) amino acid substitutions.

In some embodiments, the anti-WTMC antibody moiety comprises: (a) a HC-CDR1 comprising an amino acid sequence set forth in SEQ ID NO: 79; (b) a HC-CDR2 comprising an amino acid sequence set forth in SEQ ID NO: 80; (c) a HC-CDR3 comprising an amino acid sequence set forth in SEQ ID NO: 81; (d) a LC-CDR1 comprising an amino acid sequence set forth in SEQ ID NO: 82; (e) a LC-CDR2 comprising an amino acid sequence set forth in SEQ ID NO: 83; and (f) a LC-CDR3 comprising an amino acid sequence set forth in SEQ ID NO 84

In some embodiments, the anti-WTMC antibody moiety comprises a V_{H} comprising the amino acid sequences set forth in SEQ TD NOs: 79-81, and a V_{L} comprising the amino acid sequences set forth in SEQ ID NOs: 82-84.

The sequences of the HC-CDRs from exemplary anti-WTMC antibody clones are provided in Table B below and the LC-CDRs from exemplary anti-WTMC antibody clones are provided in Table C below.

TABLE B
Anti-WTMC antibody moiety HC-CDR sequences.

| | SEQ | | SEQ | | SEQ | |
|---|---|---|---|---|---|---|
| Clone | ID | | ID | | ID | |
| No. | NO: | HCDR1 | NO: | HCDR2 | NO: | HCDR3 |
| ESK2-1 | 1 | GYNFTSYG | 2 | ISAYNGNT | 3 | ARDWDYDFLTGWDGMDV |
| ESK2-2 | 7 | GYTFTDYY | 8 | IDPDNGGT | 9 | ARALWFGYGFLDY |
| ESK2-10 | 13 | GFTFDDYA | 14 | ISGDGGST | 15 | AKDMVAAAGWNPYYYYYGMDV |
| ESK2-11 | 19 | GYTFTGYY | 20 | INPNSGGT | 21 | ARHSSRDHYYYGMDV |
| ESK2-12 | 25 | GITVSNNY | 26 | IYSDGST | 27 | ASDKVAVAWTDGLDS |
| ESK2-14 | 31 | GYTFTDYY | 32 | VDPYDGYT | 33 | ARFSGTRQDS |
| ESK2-15 | 37 | GYTFTDFY | 38 | VNPKSGGT | 39 | ARSWQGMSWESVEDV |
| ESK2-17 | 43 | GYSFTSYW | 44 | IYPGDSDT | 45 | ARGGYIYYDA |
| ESK2-18 | 49 | GFTFSSYA | 50 | ISGSGGST | 51 | RWGWGPVGRVESHTSGDS |
| ESK2-26 | 55 | GFTFDDYA | 56 | ISWNSGSI | 57 | ARGYMGHNWYD |
| ESK2-30 | 61 | GFTFSSYW | 62 | INSDGSST | 63 | ARSGYYMSDI |
| ESK2-32 | 67 | GDTFSSYA | 68 | INLIFGTV | 69 | ARGHWSQVWWTSHSYDL |
| ESK2-34 | 73 | GYIFTGYY | 74 | INPNSGVT | 75 | AREGVWGYYDS |
| ESK2-36 | 79 | GYSFTSYW | 80 | IDPSDSYT | 81 | ARMEIYSPDY |

TABLE C
Anti-WTMC antibody moiety LC-CDR sequences.

| | SEQ | | SEQ | | SEQ | |
|---|---|---|---|---|---|---|
| Clone | ID | | ID | | ID | |
| No. | NO: | LCDR1 | NO: | LCDR2 | NO: | LCDR3 |
| ESK2-1 | 4 | SSNIGNNY | 5 | YNN | 6 | GTWDSSLSAGQV |
| ESK2-2 | 10 | SSDLGGYPF | 11 | DVT | 12 | TSYTDANTLV |
| ESK2-10 | 16 | SGGIATNY | 17 | VDN | 18 | QSYDSNNHVV |
| ESK2-11 | 22 | SSNIGNNY | 23 | ENN | 24 | GTWDSSLNAGV |
| ESK2-12 | 28 | SLRSYY | 29 | GQT | 30 | SSRDSSGNHWV |
| ESK2-14 | 34 | SSDVGGYNY | 35 | EVS | 36 | SSYAGSNNLAVV |
| ESK2-15 | 40 | SSNIGAGYD | 41 | GNS | 42 | QSYDSSLSGAV |
| ESK2-17 | 46 | RSNVGNNA | 47 | YDD | 48 | AAWDDSLNGLV |
| ESK2-18 | 52 | SSNIGAGYV | 53 | DNS | 54 | QSYDSSLSGWV |
| ESK2-26 | 58 | DIGSKA | 59 | YNR | 60 | QVWDSFSDHYV |
| ESK2-30 | 64 | NIGSKS | 65 | YDS | 66 | QVWDSSSDQYV |
| ESK2-32 | 70 | NIGSKS | 71 | DDS | 72 | QVWDSSSDHYV |
| ESK2-34 | 76 | SLRSYY | 77 | DKN | 78 | NSRDSSGNHHVV |
| ESK2-36 | 82 | QSINKY | 83 | GAL | 84 | QQSYSTPLT |

In some embodiments, the anti-WTMC antibody moiety comprises a heavy chain framework region (FR1) comprising an amino acid sequence selected from the group consisting of SEQ ID NOs: 137, 145, 153, 161, 169, 177, 185, 193, 201, 209, 217, 225, 233, and 241, a heavy chain FR2 comprising an amino acid sequence selected from the group consisting of SEQ ID NOs: 138, 146, 154, 162, 170, 178, 186, 194, 202, 210, 218, 226, 234, and 242, a heavy chain FR3 comprising an amino acid sequence selected from the group consisting of SEQ ID NOs: 139, 147, 155, 163, 171, 179, 187, 195, 203, 211, 219, 227, 235 and 243, and/or a heavy chain FR4 comprising an amino acid sequence selected from the group consisting of SEQ ID NOs: 140, 148, 156, 164, 172, 180, 188, 196, 204, 212, 220, 228, 236, and 244.

In some embodiments, the anti-WTMC antibody moiety comprises a light chain FR1 comprising an amino acid sequence selected from the group consisting of SEQ ID NOs: 141, 149, 157, 165, 173, 181, 189, 197, 205, 213, 221, 229, 237, and 245, a light chain FR2 comprising an amino acid sequence selected from the group consisting of SEQ ID NOs: 142, 150, 158, 166, 174, 182, 190, 198, 206, 214, 222, 230, 238, and 246, a light chain FR3 comprising an amino acid sequence selected from the group consisting of SEQ ID NOs: 143, 151, 159, 167, 175, 183, 191, 199, 207, 215, 223, 231, 239, and 247, and/or a light chain FR4 comprising an amino acid sequence selected from the group consisting of SEQ ID NOs: 144, 152, 160, 168, 176, 184, 192, 200, 208, 216, 224, 232, 240, and 248.

In some embodiments, the anti-WTMC antibody moiety comprises a heavy chain variable domain (VH) comprising an amino acid sequence that is at least about 85%, 90%, 95%, 96%, 97%, 98%, 99%, or 100% identical to an amino acid sequence set forth in any one of SEQ ID NOs: 85, 87, 89, 91, 93, 95, 97, 99, 101, 103, 105, 107, 109, 111, 123, 124, 125, 130, 131, 132, or 133 and/or a light chain variable domain (VL) comprising an amino acid sequence that is at least about 85%, 90%, 95%, 96%, 97%, 98%, 99%, or 100% identical to an amino acid sequence set forth in any one of SEQ ID NOs: 86, 88, 90, 92, 94, 96, 98, 100, 102, 104, 106, 108, 110, 112, 126, 127, 128, 129, 134, 135 or 136.

In some embodiments, the anti-WTMC antibody moiety comprises a VH comprising an amino acid sequence set forth in any one of SEQ ID NOs: 85, 87, 89, 91, 93, 95, 97, 99, 101, 103, 105, 107, 109, 111, 123, 124, 125, 130, 131, 132, or 133 and/or a VL comprising an amino acid sequence set forth in any one of SEQ ID NOs: 86, 88, 90, 92, 94, 96, 98, 100, 102, 104, 106, 108, 110, 112, 126, 127, 128, 129, 134, 135 or 136.

In some embodiments, the anti-WTMC antibody moiety comprising heavy chain and light chain variable domains comprising the amino acid sequence of SEQ ID NOs: 85 and 86, respectively, SEQ ID NOs: 87 and 88, respectively, SEQ ID NOs: 89 and 90, respectively, SEQ ID NOs: 91 and 92, respectively, in SEQ ID NOs: 93 and 94, respectively, SEQ ID NOs: 95 and 96, respectively, SEQ ID NOs: 97 and 98, respectively, SEQ ID NOs: 99 and 100, respectively, SEQ ID NOs: 101 and 102, respectively, in SEQ ID NOs: 103 and 104, respectively, SEQ ID NOs: 105 and 106, respectively, SEQ ID NOs: 107 and 108, respectively, SEQ ID NOs: 109 and 110, respectively, SEQ ID NOs: 111 and 112, respectively, SEQ ID NOs: 123 and 102, respectively, SEQ ID NOs: 124 and 102, respectively, SEQ ID NOs: 125 and 102, respectively, SEQ ID NOs: 123 and 126, respectively, SEQ ID NOs: 124 and 126, respectively, SEQ ID NOs: 125 and 126, respectively, SEQ ID NOs: 123 and 127, respectively, SEQ ID NOs: 124 and 127, respectively, SEQ ID NOs: 125 and 127, respectively, SEQ ID NOs: 123 and 128, respectively, SEQ ID NOs: 124 and 128, respectively, SEQ ID NOs: 125 and 128, respectively, SEQ ID NOs: 123 and 129, respectively, SEQ ID NOs: 124 and 129, respectively, SEQ ID NOs: 125 and 129, respectively, SEQ ID NOs: 101 and 126, respectively, SEQ ID NOs: 101 and 127, respectively, SEQ ID NOs: 101 and 128, respectively, SEQ ID NOs: 101 and 129, respectively, SEQ ID NOs: 130 and 110, respectively, SEQ ID NOs: 130 and 134, respectively, SEQ ID NOs: 130 and 135, respectively, SEQ ID NOs: 130 and 136, respectively, SEQ ID NOs: 131 and 110, respectively, SEQ ID NOs: 131 and 134, respectively, SEQ ID NOs: 131 and 135, respectively, SEQ ID NOs: 131 and 136, respectively, SEQ ID NOs: 132 and 110, respectively, SEQ ID NOs: 132 and 134, respectively, SEQ ID NOs: 132 and 135, respectively, SEQ ID NOs: 132 and 136, respectively, SEQ ID NOs: 133 and 110, respectively, SEQ ID NOs: 133 and 134, respectively, SEQ ID NOs: 133 and 135, respectively, SEQ ID NOs: 133 and 136, respectively, SEQ ID NOs: 109 and 134, respectively, SEQ ID NOs: 109 and 135, respectively, SEQ ID NOs: 109 and 136, respectively; or variants thereof having, individually, at least about 95% (for example at least about any of 96%, 97%, 98%, or 99%) sequence identity.

The sequences of the heavy chain variable domains and light chain variable domains from exemplary anti-WTMC antibody clones are provided in Table 4 below.

| SEQ | |
|---|---|
| ID | |
| NO | |
| | V_{H} sequences |
| 85 | EVQLVQSGAEVKKPGASVKVSCKASGYNFTSYGISWVRQAPGQGLEWMGWISAYNGNTNY |
| | AQKLQGRVTMTTDTSTSTAYMELRSLRSDDTAVYYCARDWDYDFLTGWDGMDVWGQGTT |
| | VTVSS |
| 87 | QVQLVQSGAEVKKPGASVKVSCKASGYTFTDYYIHWVRQAPGQGLEWMGWIDPDNGGTNY |
| | AQNFQDRVTMTRDTSVSTAYLEVTSLKSDDAAVYYCARALWFGYGFLDYHWGQGTLVTVS |
| | S |
| 89 | EVQLVESGGGVVQPGGSLRLSCAASGFTFDDYAMHWVRQAPGKGLEWVSLISGDGGSTYYA |
| | DSVKGRFTISRDNSKNSLYLQMNSLRTEDTALYYCAKDMVAAAGWNPYYYYYGMDVWGQ |
| | GTTVTVSS |
| 91 | EVQLVQSGAEVKKPGASVKVSCKASGYTFTGYYMHWVRQAPGQGLEWMGWINPNSGGTN |
| | YAQKFQGRVTMTRDTSISTAYMELGRLRSDDTAVYYCARHSSRDHYYYGMDVWGQGTTVT |
| | VSS |
| 93 | EVQLVESGGGLVQPGGSLRLSCVVSGITVSNNYMTWVRQAPGKGLEWVSVIYSDGSTYYADS |
| | VRGRFTISRDISKNTVFLQMNSLRAEDTAMYYCASDKVAVAWTDGLDSWGQGTMVTVSS |
| 95 | EVQLVQSGAEVKKPGASVKISCKTSGYTFTDYYLHWVRQAPVQGLEWMGYVDPYDGYTHY |
| | AQNFQGRVTMTTDTSTSTAYMELSSLRSEDTAVYYCARFSGTRQDSHWGQGTLVTVSS |
| 97 | QVQLVQSGAEVKKPGASVKVSCKASGYTFTDFYIHWVRQAPGQGPEWMGWVNPKSGGTNY |
| | AQKFQGRVTMTRDTSISTAYMALSRLRSDDTAVYYCARSWQGMSWESVEDVWGQGTLVTV |
| | SS |
| 99 | EVQLVQSGAEVKKPGESLKISCKGSGYSFTSYWIGWVRQMPGKGLEWMGIIYPGDSDTRYSP |
| | SFQGQVTISADKSISTAYLQWSSLKASDTAVYYCARGGYIYYDAWGQGTLVTVSS |
| 101 | EVQLVESGGGLVQPGGSLRLSCAASGFTFSSYAMSWVRQAPGKGLEWVSAISGSGGSTYYAD |
| | SVKGRFTISRDNSKNTLYLQMNSLRAEDTAVYYCARWGWGPVGRVESHTSGDSWGQGTLVT |
| | VSS |
| 103 | QVQLVQSGGGLVQPGRSLRLSCAASGFTFDDYAMHWVRQAPGKGLEWVSGISWNSGSIGYA |
| | DSVKGRFTISRDNAKNSLYLQMNSLRPEDTAVYYCARGYMGHNWYDWGQGTLVTVSS |
| 105 | QVQLQESGGGLVQPGGSLRLSCAASGFTFSSYWMHWVRQAPGKGLVWVSRINSDGSSTSYA |
| | DSVKGRFTISRDNAKNTLYLQMNSLRAEDTAVYYCARSGYYMSDIWGQGTLVTVSS |
| 107 | QVQLVQSGAEVKKPGSSVKVSCKASGDTFSSYAVSWVRQAPGQGLEWMGAINLIFGTVKYA |
| | QKFQGRITITADESTSTAYMELSSLRSEDTAVYYCARGHWSQVWWTSHSYDLWGQGTLVTV |
| | SS |
| 109 | QVQLVQSGTEVRKPGASLKVSCTTSGYIFTGYYIHWVRQVPGQGLEWMGWINPNSGVTEFAQ |
| | GFQGRITMTRDTSTSTVYMELSRLTSDDTAVYYCAREGVWGYYDSWGQGTLVTVSS |
| 111 | EVQLVQSGAEVKKPGESLRISCKDSGYSFTSYWISWVRQMPGKGLEWMGRIDPSDSYTNYSP |
| | SFQGHVTISADKSISTAYLQWSSLKASDTAMYYCARMEIYSPDYWGQGTLVTVSS |
| 123 | EVQLLESGGGLVQPGGSLRLSCAASGFTFSSYAMSWVRQAPGKGLEWVSAISGSGGSTYYGD |
| | SVKGRFTISRDNSKNTLYLQMNSLRAEDTAVYYCARWGWGPVGRVESHTSGDSWGQGTLVT |
| | VSS |
| 124 | EVQLVESGGGLVQPGGSLRLSCAASGFTFSSYAMSWVRQAPGKGLEWVSAISGSGGSTYYGD |
| | SVKGRFTISRDNSKNTLYLQMNSLRAEDTAVYYCARWGWGPVGRVESHTSGDSWGQGTLVT |
| | VSS |
| 125 | EVQLLESGGGLVQPGGSLRLSCAASGFTFSSYAMSWVRQAPGKGLEWVSAISGSGGSTYYAD |
| | SVKGRFTISRDNSKNTLYLQMNSLRAEDTAVYYCARWGWGPVGRVESHTSGDSWGQGTLVT |
| | VSS |
| 130 | QVQLVQSGAEVKKPGASLKVSCTTSGYIFTGYYIHWVRQAPGQGLEWMGWINPNSGVTEFA |
| | QGFQGRITMTRDTSTSTAYMELSRLTSDDTAVYYCAREGVWGYYDSWGQGTLVTVSS |
| 131 | QVQLVQSGTEVKKPGASLKVSCTTSGYIFTGYYIHWVRQVPGQGLEWMGWINPNSGVTEFA |
| | QGFQGRITMTRDTSTSTAYMELSRLTSDDTAVYYCAREGVWGYYDSWGQGTLVTVSS |
| 132 | QVQLVQSGAEVRKPGASLKVSCTTSGYIFTGYYIHWVRQAPGQGLEWMGWINPNSGVTEFA |
| | QGFQGRITMTRDTSTSTVYMELSRLTSDDTAVYYCAREGVWGYYDSWGQGTLVTVSS |
| 133 | QVQLVQSGTEVKKPGASLKVSCTTSGYIFTGYYIHWVRQAPGQGLEWMGWINPNSGVTEFA |
| | QGFQGRITMTRDTSTSTAYMELSRLTSDDTAVYYCAREGVWGYYDSWGQGTLVTVSS |
| | V_{L} sequences |
| 86 | QSVVTQPPSVSAAPGQKVTISCSGSSSNIGNNYVSWYQQLPGTAPKLLIYYNNKRPSGIPDRFS |
| | GSKSGTSATLGITGLQTGDEADYYCGTWDSSLSAGQVFGGGTKLTVLG |
| 88 | QSALTQPASVSGSPGQSITISCTGSSSDLGGYPFVSWYQQHPDKAPKLIIYDVTNRPSGISNRFS |
| | GSKSGYRASLTISGLQAEDEADYYCTSYTDANTLVFGTGTKVTVLG |
| 90 | NFMLTQPHSVSESPGKTVTISCTRSSGGIATNYVQWYQQRPGNAPTAVIYVDNERPSGVPERFS |
| | GSIDTSSNSASLTISGLETEDEADYYCQSYDSNNHVVFGGGTKVTVLG |
| 92 | QSVVTQPPSVSAAPGQRVTISCSGGSSNIGNNYVSWYQHLPGTAPKLLIYENNLKRPSGIPDRF |
| | SGSKSGTSATLGITGLQTGDEADYYCGTWDSSLNAGVFGGGTKLTVLG |
| 94 | SSELTQDPTVSVALGQTVRITCQGDSLRSYYGGWYQQKPGQAPILVFHGQTNRPSGIPDRFSGS |
| | SSGNTVSLTITGAQAEDEADYYCSSRDSSGNHWVFGGGTKLTVLG |
| 96 | QSALTQPPSASGSPGQSVTISCTGTSSDVGGYNYVSWYQQHPGKAPKLMIYEVSKRPSGVPDR |
| | FSGSKSGNTASLTVSGLQAEDEADYYCSSYAGSNNLAVVFGGGTKLTVLG |
| 98 | QSVLTQPPSVSGAPGQRVTISCTGSSSNIGAGYDVHWYQQLPGTAPKLLIYGNSNRPSGVPDRF |
| | SGSKSGTSASLAITGLQAEDEADYYCQSYDSSLSGAVFGTGTKVTVLG |
| 100 | SYVLTQPPSVSEAPRQRVTISCSGSRSNVGNNAVSWYQHLPGKAPKLLIYYDDLMPSGVSDRF |
| | SGSKSGTSASLAISGLQSEDEADYYCAAWDDSLNGLVFGGGTKLTVLG |
| 102 | QSVLTQPPSVSGAPGQRVSISCTGSSSNIGAGYVVHWYQHLPGTVPKLLIYDNSNRPSGVPDRF |
| | SGSNSGSSASLAVTGLQAEDEADYYCQSYDSSLSGWVFGGGTKLTVLG |
| 104 | LPVLTQPPSVSVAPGKTARITCGGNDIGSKAVHWYQQKPGQAPVLVIYYNRDRPSGIPERFSGS |
| | NSGNTATLTISRVEAGDEADYYCQVWDSFSDHYVFGTGTKVTVLG |
| 106 | NFMLTQPPSLSVAPGKTATITCGGNNIGSKSVHWYQQKPGQAPVLVISYDSDRPSGIPERFSGS |
| | KSGNTATLTISRVEAGDEADYYCQVWDSSSDQYVFGSGTKVTVLG |
| 108 | QAVLTQPPSVSVAPGKTARITCGGNNIGSKSVHWYQQKPGQAPVLVVYDDSDRPSGIPERFSG |
| | SNSGNTATLTISRVEAGDEADYYCQVWDSSSDHYVFGTGTKVTVLG |
| 110 | SSELTQDPAVSVALGQTVRITCQGDSLRSYYASWYQQKPGQAPVLVIYDKNNRPSGIPDRFSG |
| | SSSGNTASLTITGAQAEDEADYYCNSRDSSGNHHVVFGGGTKLTVLG |
| 112 | DIQLTQSPSSLSASVGDRVTISCRASQSINKYLNWYQQKPGEAPKLLIYGALRLQSGVPSRFSGS |
| | GSGTDYALTITSLQPEDFATYYCQQSYSTPLTFGGGTKVDIKR |
| 126 | QSVLTQPPSVSGAPGQRVTISCTGSSSNIGAGYVVHWYQQLPGTAPKLLIYDNSNRPSGVPDR |
| | FSGSKSGTSASLAVTGLQAEDEADYYCQSYDSSLSGWVFGGGTKLTVL |
| 127 | QSVLTQPPSVSGAPGQRVTISCTGSSSNIGAGYVVHWYQHLPGTAPKLLIYDNSNRPSGVPDRF |
| | SGSNSGSSASLAVTGLQAEDEADYYCQSYDSSLSGWVFGGGTKLTVL |
| 128 | QSVLTQPPSVSGAPGQRVSISCTGSSSNIGAGYVVHWYQQLPGTVPKLLIYDNSNRPSGVPDRF |
| | SGSNSGTSASLAVTGLQAEDEADYYCQSYDSSLSGWVFGGGTKLTVL |
| 129 | QSVLTQPPSVSGAPGQRVTISCTGSSSNIGAGYVVHWYQHLPGTVPKLLIYDNSNRPSGVPDRF |
| | SGSKSGTSASLAVTGLQAEDEADYYCQSYDSSLSGWVFGGGTKLTVL |
| 134 | SYELTQDPAVSVALGQTVRITCQGDSLRSYYASWYQQKPGQAPVRVIYDKNNRPSGIPDRFSG |
| | SSSGNTASLTITGAQAEDEADYYCNSRDSSGNHHVVFGGGTKLTVL |
| 135 | SYELTQDPAVSVALGQTVRITCQGDSLRSYYASWYQQKPGQAPVLVIYDKNNRPSGIPDRFSG |
| | SSSGNTASLTITGAQAEDEADYYCNSRDSSGNHHVVFGGGTKLTVL |
| 136 | SSELTQDPAVSVALGQTVRITCQGDSLRSYYASWYQQKPGQAPVRVIYDKNNRPSGIPDRFSG |
| | SSSGNTASLTITGAQAEDEADYYCNSRDSSGNHHVVFGGGTKLTVL |

The heavy and light chain variable domains can be combined in pair-wise combinations to generate additional anti-WTMC antibody moieties that can be incorporated into and/or used with the anti-WTMC constructs of the present disclosure.

In some embodiments according to any of the anti-WTMC constructs described above, the antibody moiety is chimeric, humanized, partially human, fully human, or semi-synthetic. In some embodiments, the antibody moiety is a full-length antibody, a Fab, a Fab′, a F(ab′)₂, an Fv, or a single chain Fv (scFv). In some embodiments, the antibody moiety is an scFv. Exemplary scFv amino acid sequences include, but are not limited to, SEQ ID NOs: 335-348. A person of ordinary skill in the art would understand that the V_{H} and V_{L} domains can be combined in different pair-wise combinations, different orders and linker sequences to generate additional anti-WTMC scFvs that can be incorporated into and/or used with the anti-WTMC constructs of the present disclosure.

The anti-WTMC antibodies or antibody moieties may be identified by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al., Methods in Molecular Biology 178:1-37 (O'Brien et al., ed., Human Press, Totowa, N.J., 2001) and further described, e.g., in McCafferty et al., Nature 348:552-554; Clackson et al., Nature 352: 624-628 (1991); Marks et al., J. Mol. Biol. 222: 581-597 (1992); Marks and Bradbury, Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natd. Acad. Sci. USA 101(34): 12467-12472 (2004); and Lee et al., J. Immunol. Methods 284(1-2): 119-132(2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naïve repertoire can be cloned (e.g., from human) to provide a single source of antibodies to a wide range of non-self and also self antigens without any immunization as described by Griffiths et al., EMBO J, 12: 725-734 (1993). Finally, naïve libraries can also be made synthetically by cloning unrearranged V-gene segments from stem cells and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

The antibodies or antigen-binding fragments thereof can be prepared using phage display to screen libraries for antibodies specific to a complex comprising a WT1 peptide and an MHC class I protein. The library can be a human scFv phage display library having a diversity of at least one×10⁹ (such as at least about any of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naïve human library constructed from DNA extracted from human PMBCs and spleens from healthy donors, encompassing all human heavy and light chain subfamilies. In some embodiments, the library is a naïve human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see, e.g., Hoet, R. M. et al., Nat. Biotechnol. 23(3):344-348, 2005). In some embodiments, the heavy chain CDR3 of the semi-synthetic human library has a length from about 5 to about 24 (such as about any of 5, 6, 7, 8, 9, 10, 11, 12, 13, 14, 15, 16, 17, 18, 19, 20, 21, 22, 23, or 24) amino acids. In some embodiments, the library is a non-human phage display library.

Phage clones that bind to the WTMC with high affinity can be selected by iterative binding of phage to the WTMC, which is bound to a solid support (such as, for example, beads for solution panning or mammalian cells for cell panning), followed by removal of non-bound phage and by elution of specifically bound phage. In an example of solution panning, the WTMC can be biotinylated for immobilization to a solid support. The biotinylated WTMC is mixed with the phage library and a solid support, such as streptavidin-conjugated Dynabeads™ M-280 (ThermoFisher), and then WTMC-phage-bead complexes are isolated. The bound phage clones are then eluted and used to infect an appropriate host cell, such as E. coli XL1-Blue, for expression and purification. In an example of cell panning, T2 cells (a TAP-deficient, HLA-A*02:01⁺ lymphoblast cell line) loaded with the WT1 peptide of the WTMC are mixed with the phage library, after which the cells are collected, and the bound clones are eluted and used to infect an appropriate host cell for expression and purification. The panning can be performed for multiple (such as about any of 2, 3, 4, 5, 6 or more) rounds with either solution panning, cell panning, or a combination of both, to enrich for phage clones binding specifically to the WTMC. Enriched phage clones can be tested for specific binding to the WTMC by any methods known in the art, including for example ELISA and FACS.

### MHC Class I Proteins

MHC class I proteins are one of two primary classes of major histocompatibility complex (MHC) molecules (the other being MHC class II) and are found on nearly every nucleated cell of the body. Their function is to display fragments of proteins from within the cell to T cells; healthy cells will be ignored, while cells containing foreign proteins will be attacked by the immune system. Because MHC class I proteins present peptides derived from cytosolic proteins, the pathway of MHC class I presentation is often called the cytosolic or endogenous pathway. Class I MHC molecules bind peptides generated mainly from degradation of cytosolic proteins by the proteasome. The MHC I:peptide complex is then inserted into the plasma membrane of the cell. The peptide is bound to the extracellular part of the class I MHC molecule. Thus, the function of the class I MHC is to display intracellular proteins to cytotoxic T cells (CTLs). However, class I MHC can also present peptides generated from exogenous proteins, in a process known as cross-presentation.

MHC class I proteins consist of two polypeptide chains, α and β2-microglobulin (β2M). The two chains are linked noncovalently via interaction of β2M and the α3 domain. Only the α chain is polymorphic and encoded by a HLA gene, while the β2M subunit is not polymorphic and encoded by the J-2 microglobulin gene. The α3 domain is plasma membrane-spanning and interacts with the CD8 co-receptor of T cells. The α3-CD8 interaction holds the MHC I molecule in place while the T cell receptor on the surface of the cytotoxic T cell binds its α1-α2 heterodimer ligand and checks the coupled peptide for antigenicity. The α1 and α2 domains fold to make up a groove for peptides to bind. MHC class I proteins bind peptides that are 8-10 amino acids in length.

The human leukocyte antigen (HLA) genes are the human versions of the MHC genes. The three major MHC class I proteins in humans are HLA-A, HLA-B, and HLA-C, while the 3 minor ones are HLA-E, HLA-F, and HLA-G. HLA-A is ranked among the genes in humans with the fastest-evolving coding sequence. As of December 2013, there were 2432 known HLA-A alleles coding for 1740 active proteins and 117 null proteins. The HLA-A gene is located on the short arm of chromosome 6 and encodes the larger, α-chain, constituent of HLA-A. Variation of HLA-A α-chain is key to HLA function. This variation promotes genetic diversity in the population. Since each HLA has a different affinity for peptides of certain structures, greater variety of HLAs means greater variety of antigens to be ‘presented’ on the cell surface, enhancing the likelihood that a subset of the population will be resistant to any given foreign invader. This decreases the likelihood that a single pathogen has the capability to wipe out the entire human population. Each individual can express up to two types of HLA-A, one from each of their parents. Some individuals will inherit the same HLA-A from both parents, decreasing their individual HLA diversity; however, the majority of individuals will receive two different copies of HLA-A. This same pattern follows for all HLA groups. In other words, a person can only express either one or two of the 2432 known HLA-A alleles.

All alleles receive at least a four-digit classification, e.g., HLA-A*02:12. The A signifies which HLA gene the allele belongs to. There are many HLA-A alleles, so that classification by serotype simplifies categorization. The next pair of digits indicates this assignment. For example, HLA-A*02:02, HLA-A*02:04, and HLA-A*02:324 are all members of the A2 serotype (designated by the *02 prefix). This group is the primary factor responsible for HLA compatibility. All numbers after this cannot be determined by serotyping and are designated through gene sequencing. The second set of digits indicates what HLA protein is produced. These are assigned in order of discovery and as of December 2013 there are 456 different HLA-A02 proteins known (assigned names HLA-A*02:01 to HLA-A*02:456). The shortest possible HLA name includes both of these details. Each extension beyond that signifies a nucleotide change that may or may not change the protein.

In some embodiments, the anti-WTMC antibody moiety specifically binds to a complex comprising a WT1 peptide (e.g., WT1-RMF) and an MHC class I protein, wherein the MHC class I protein is HLA-A, HLA-B, HLA-C, HLA-E, HLA-F, or HLA-G. In some embodiments, the MHC class I protein is HLA-A, HLA-B, or HLA-C. In some embodiments, the MHC class I protein is HLA-A. In some embodiments, the MHC class I protein is HLA-B. In some embodiments, the MHC class I protein is HLA-C. In some embodiments, the MHC class I protein is HLA-A01, HLA-A02, HLA-A03, HLA-A09, HLA-A10, HLA-A11, HLA-A19, HLA-A23, HLA-A24, HLA-A25, HLA-A26, HLA-A28, HLA-A29, HLA-A30, HLA-A31, HLA-A32, HLA-A33, HLA-A34, HLA-A36, HLA-A43, HLA-A66, HLA-A68, HLA-A69, HLA-A74, or HLA-A80. In some embodiments, the MHC class I protein is HLA-A02. In some embodiments, the MHC class I protein is any one of HLA-A*02:01-555, such as HLA-A*02:01, HLA-A*02:02, HLA-A*02:03, HLA-A*02:04, HLA-A*02:05, HLA-A*02:06, HLA-A*02:07, HLA-A*02:08, HLA-A*02:09, HLA-A*02:10, HLA-A*02:11, HLA-A*02:12, HLA-A*02:13, HLA-A*02:14, HLA-A*02:15, HLA-A*02:16, HLA-A*02:17, HLA-A*02:18, HLA-A*02:19, HLA-A*02:20, HLA-A*02:21, HLA-A*02:22, or HLA-A*02:24. In some embodiments, the MHC class I protein is HLA-A*02:01. HLA-A*02:01 is expressed in 39-46% of all Caucasians, and therefore represents a suitable choice of MHC class I protein for use in the present application.

WT1 peptides suitable for use in generating anti-WTMC antibody moieties can be determined, for example, based on the presence of HLA-A*02:01-binding motifs and cleavage sites for proteasomes and immune-proteasomes using computer prediction models known to those of skill in the art. For predicting MHC binding sites, such models include, but are not limited to, IEDB (Vita et al., The immune epitope database (IEDB) 3.0. Nucleic Acids Res. 2014 Oct. 9. pii: gku938), ProPred1 (described in more detail in Singh and Raghava, ProPred: prediction of HLA-DR binding sites. BIOINFORMATICS 17(12):1236-1237, 2001), and SYFPEITHI (see Schuler et al. SYFPEITHI, Database for Searching and T-Cell Epitope Prediction. in Immunoinformatics Methods in Molecular Biology, vol 409(1): 75-93, 2007).

Once appropriate peptides have been identified, peptide synthesis may be done in accordance with protocols well known to those of skill in the art. Because of their relatively small size, the peptides of the present application may be directly synthesized in solution or on a solid support in accordance with conventional peptide synthesis techniques. Various automatic synthesizers are commercially available and can be used in accordance with known protocols. The synthesis of peptides in solution phase has become a well-established procedure for large-scale production of synthetic peptides and as such is a suitable alternative method for preparing the peptides of the present application (See for example, Solid Phase Peptide Synthesis by John Morrow Stewart and Martin et al. Application of Almez-mediated Amidation Reactions to Solution Phase Peptide Synthesis, Tetrahedron Letters Vol. 39, pages 1517-1520, 1998).

The binding activity of candidate WT1 peptides (e.g., WT1-RMF) can be tested using the antigen-processing-deficient T2 cell line, which increases expression of HLA-A when stabilized by a peptide in the antigen-presenting groove. T2 cells are pulsed with the candidate peptide for a time sufficient to stabilize HLA-A expression on the cell surface, which can be measured using any methods known in the art, such as by immunostaining with a fluorescently labeled monoclonal antibody specific for HLA-A (for example, BB7.2) followed by fluorescence-activated cell-sorting (FACS) analysis.

### Monoclonal Anti-WTMC Antibodies and Antibody Moieties

In some embodiments, an anti-WTMC construct of the present disclosure comprises a monoclonal anti-WTMC antibody or a monoclonal anti-WTMC antibody moiety. Monoclonal antibodies can be prepared, e.g., using hybridoma methods, such as those described by Kohler and Milstein, Nature, 256:495 (1975) and Sergeeva et al., Blood, 117(16):4262-4272, using the phage display methods described herein and in the Examples below, or using recombinant DNA methods (see, e.g., U.S. Pat. No. 4,816,567).

In a hybridoma method, a hamster, mouse, or other appropriate host animal is typically immunized with an immunizing agent to elicit lymphocytes that produce or are capable of producing antibodies that will specifically bind to the immunizing agent. Alternatively, the lymphocytes can be immunized in vitro. The immunizing agent can include a polypeptide or a fusion protein of the protein of interest, or a complex comprising at least two molecules. Generally, peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. See, e.g., Goding, Monoclonal Antibodies: Principles and Practice (New York: Academic Press, 1986), pp. 59-103. Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which prevents the growth of HGPRT-deficient cells.

In some embodiments, the immortalized cell lines fuse efficiently, support stable high-level expression of antibody by the selected antibody-producing cells, and are sensitive to a medium such as HAT medium. In some embodiments, the immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of human monoclonal antibodies. Kozbor, J. Immunol., 133:3001 (1984); Brodeur et al. Monoclonal Antibody Production Techniques and Applications (Marcel Dekker, Inc.: New York, 1987) pp. 51-63.

The culture medium in which the hybridoma cells are cultured can then be assayed for the presence of monoclonal antibodies directed against the polypeptide. The binding specificity of monoclonal antibodies produced by the hybridoma cells can be determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be sub cloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the sub clones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

In certain embodiments, the anti-WTMC antibody or antibody moiety is monovalent. Methods for preparing monovalent antibodies are known in the art. One exemplary method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using any method known in the art.

Antibody variable domains with the desired binding specificities (antibody-antigen combining sites) can be fused to immunoglobulin constant-domain sequences. The fusion preferably is with an immunoglobulin heavy-chain constant domain, comprising at least part of the hinge, CH2, and CH3 regions. In some embodiments, the first heavy-chain constant region (CH1) containing the site necessary for light-chain binding is present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies, see, for example, Suresh et al., Methods in Enzymology, 121: 210 (1986).

Monoclonal antibodies can also be made by recombinant DNA methods, such as those described in U.S. Pat. No. 4,816,567. DNA encoding the monoclonal anti-WTMC antibodies can be readily isolated and sequenced using conventional procedures (e.g., by using oligonucleotide probes that are capable of binding specifically to genes encoding the heavy and light chains of murine antibodies). Hybridoma cells as described above or WTMC-specific phage clones can serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains and/or framework regions in place of the homologous non-human sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an anti-WTMC antibody or can be substituted for the variable domains of one antigen-combining site of an anti-WTMC antibody to create a chimeric bivalent antibody.

### Human and Humanized Anti-WTMC Antibodies and Antibody Moieties, and Preparation Thereof

The anti-WTMC antibodies or antibody moieties can be humanized antibodies or human antibodies. Humanized forms of non-human (e.g., murine) antibodies are chimeric immunoglobulins, immunoglobulin chains, or fragments thereof (such as Fv, Fab, Fab′, F(ab′)₂, scFv, or other antigen-binding subsequences of antibodies) that typically contain minimal sequence derived from non-human immunoglobulin.

Humanized antibodies include human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody can comprise substantially all of at least one, and typically two, variable domains, in which all or substantially all of the CDR regions correspond to those of a non-human immunoglobulin, and all or substantially all of the FR regions are those of a human immunoglobulin consensus sequence. In some embodiments, the humanized antibody will comprise at least a portion of an immunoglobulin constant region (Fc), typically that of a human immunoglobulin. See, e.g., Jones et al., Nature, 321: 522-525 (1986); Riechmann et al., Nature, 332: 323-329 (1988); Presta, Curr. Op. Struct. Biol., 2:593-596 (1992).

Generally, a humanized antibody has one or more amino acid residues introduced into it from a source that is non-human. These non-human amino acid residues are often referred to as “import” residues, which are typically taken from an “import” variable domain. According to some embodiments, humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody. Accordingly, such “humanized” antibodies are antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., PNAS USA, 90:2551 (1993); Jakobovits et al., Nature, 362:255-258 (1993); Bruggemann et al., Year in Immunol., 7:33 (1993); U.S. Pat. Nos. 5,545,806, 5,569,825, 5,591,669; 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and Marks et al., Bio Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Human antibodies may also be generated by in vitro activated B cells (see U.S. Pat. Nos. 5,567,610 and 5,229,275) or by using various techniques known in the art, including phage display libraries. Hoogenboom and Winter, J. Mol. Biol., 227:381 (1991); Marks et al., J. Mol. Biol., 222:581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

### Full-Length Anti-WTMC Antibodies

In some embodiments, the anti-WTMC construct provided herein is or comprises a full-length antibody, e.g., a full-length antibody comprising an anti-WTMC antibody moiety, also referred to herein as a “full-length anti-WTMC antibody.” In some embodiments, the full-length antibody is a monoclonal antibody, as described in further detail elsewhere herein.

In some embodiments, the full-length anti-WTMC antibody comprises an Fc sequence from an immunoglobulin, e.g., a human immunoglobulin such as IgA, IgD, IgE, IgG, or IgM. In some embodiments, the full-length anti-WTMC antibody comprises an Fc sequence of IgG, e.g., a human IgG, such as any of IgG1, IgG2, IgG3, or IgG4. In some embodiments, the full-length anti-WTMC antibody comprises an Fc sequence of a rabbit, rat, or mouse immunoglobulin. In some embodiments, the full-length anti-WTMC antibody comprises an Fc sequence of a non-human primate (e.g., a rhesus monkey or cynomolgus monkey). In some embodiments, the full-length anti-WTMC antibody comprises an Fc sequence that has been altered or otherwise changed so that it has enhanced antibody dependent cellular cytotoxicity (ADCC) function and/or enhanced complement dependent cytotoxicity (CDC) effector function, as described in further detail elsewhere herein.

Thus, for example, in some embodiments, there is provided a full-length anti-WTMC antibody comprising a) any one of the anti-WTMC antibody moieties described herein that specifically binds to a complex comprising a WT1 peptide (e.g., WT1-RMF) and an MHC class I protein, and b) an Fc region. In some embodiments, the WT1 peptide (e.g., WT1-RMF) comprises the amino acid sequence of SEQ ID NO: 113. In some embodiments, the antibody moiety specifically recognizes amino acid residues FPNA (SEQ ID NO: 349) of SEQ ID NO: 113. In some embodiments, the MHC class I protein is HLA-A02. In some embodiments, the MHC class I protein is HLA-A*02:01. In some embodiments, the Fc region comprises an IgG1 Fc sequence. In some embodiments, the Fc region comprises a human IgG1 Fc sequence. In some embodiments, the Fc region comprises a mouse IgG1 Fc sequence.

In some embodiments, there is provided a full-length anti-WTMC antibody comprising a) any one of the anti-WTMC antibody moieties described herein, and b) an Fc region. In some embodiments, the Fc region comprises an IgG1 Fc sequence. In some embodiments, the Fc region comprises a human IgG1 Fc sequence. In some embodiments, the Fc region comprises a mouse IgG1 Fc sequence.

In some embodiments, the full-length anti-WTMC IgG1 antibody comprises: (a) a HC-CDR1 comprising an amino acid sequence set forth in SEQ ID NO: 1; (b) a HC-CDR2 comprising an amino acid sequence set forth in SEQ ID NO: 2; (c) a HC-CDR3 comprising an amino acid sequence set forth in SEQ ID NO: 3; (d) a LC-CDR1 comprising an amino acid sequence set forth in SEQ ID NO: 4; (e) a LC-CDR2 comprising an amino acid sequence set forth in SEQ ID NO: 5; and (f) a LC-CDR3 comprising an amino acid sequence set forth in SEQ ID NO: 6. In some embodiments, the CDRs are human CDRs. In some embodiments, the full-length anti-WTMC IgG1 antibody comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 85 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 86. In some embodiments, the full-length anti-WTMC IgG1 antibody comprises a V_{H} domain comprising SEQ ID NO: 85 and a V_{L} domain comprising SEQ ID NO: 86.

In some embodiments, the full-length anti-WTMC IgG1 antibody comprises: (a) a HC-CDR1 comprising an amino acid sequence set forth in SEQ ID NO: 7; (b) a HC-CDR2 comprising an amino acid sequence set forth in SEQ ID NO: 8; (c) a HC-CDR3 comprising an amino acid sequence set forth in SEQ ID NO: 9; (d) a LC-CDR1 comprising an amino acid sequence set forth in SEQ ID NO: 10; (e) a LC-CDR2 comprising an amino acid sequence set forth in SEQ ID NO: 11; and (f) a LC-CDR3 comprising an amino acid sequence set forth in SEQ ID NO: 12. In some embodiments, the CDRs are human CDRs. In some embodiments, the full-length anti-WTMC IgG1 antibody comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 87 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 88. In some embodiments, the full-length anti-WTMC IgG1 antibody comprises a V_{H} domain comprising SEQ ID NO: 87 and a V_{L} domain comprising SEQ ID NO: 88.

In some embodiments, the full-length anti-WTMC IgG1 antibody comprises: (a) a HC-CDR1 comprising an amino acid sequence set forth in SEQ ID NO: 13; (b) a HC-CDR2 comprising an amino acid sequence set forth in SEQ ID NO: 14; (c) a HC-CDR3 comprising an amino acid sequence set forth in SEQ ID NO: 15; (d) a LC-CDR1 comprising an amino acid sequence set forth in SEQ ID NO: 16; (e) a LC-CDR2 comprising an amino acid sequence set forth in SEQ ID NO: 17; and (f) a LC-CDR3 comprising an amino acid sequence set forth in SEQ ID NO: 18. In some embodiments, the CDRs are human CDRs. In some embodiments, the full-length anti-WTMC IgG1 antibody comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 89 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 90. In some embodiments, the full-length anti-WTMC IgG1 antibody comprises a V_{H} domain comprising SEQ ID NO: 89 and a V_{L} domain comprising SEQ ID NO: 90.

In some embodiments, the full-length anti-WTMC IgG1 antibody comprises: (a) a HC-CDR1 comprising an amino acid sequence set forth in SEQ ID NO: 19; (b) a HC-CDR2 comprising an amino acid sequence set forth in SEQ ID NO: 20; (c) a HC-CDR3 comprising an amino acid sequence set forth in SEQ ID NO: 21; (d) a LC-CDR1 comprising an amino acid sequence set forth in SEQ ID NO: 22; (e) a LC-CDR2 comprising an amino acid sequence set forth in SEQ ID NO: 23; and (f) a LC-CDR3 comprising an amino acid sequence set forth in SEQ ID NO: 24. In some embodiments, the CDRs are human CDRs. In some embodiments, the full-length anti-WTMC IgG1 antibody comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 91 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 92. In some embodiments, the full-length anti-WTMC IgG1 antibody comprises a V_{H} domain comprising SEQ ID NO: 91 and a V_{L} domain comprising SEQ ID NO: 92.

In some embodiments, the full-length anti-WTMC IgG1 antibody comprises: (a) a HC-CDR1 comprising an amino acid sequence set forth in SEQ ID NO: 25; (b) a HC-CDR2 comprising an amino acid sequence set forth in SEQ ID NO: 26; (c) a HC-CDR3 comprising an amino acid sequence set forth in SEQ ID NO: 27; (d) a LC-CDR1 comprising an amino acid sequence set forth in SEQ ID NO: 28; (e) a LC-CDR2 comprising an amino acid sequence set forth in SEQ ID NO: 29; and (f) a LC-CDR3 comprising an amino acid sequence set forth in SEQ ID NO: 30. In some embodiments, the CDRs are human CDRs. In some embodiments, the full-length anti-WTMC IgG1 antibody comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 93 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 94. In some embodiments, the full-length anti-WTMC IgG1 antibody comprises a V_{H} domain comprising SEQ ID NO: 93 and a V_{L} domain comprising SEQ ID NO: 94.

In some embodiments, the full-length anti-WTMC IgG1 antibody comprises: (a) a HC-CDR1 comprising an amino acid sequence set forth in SEQ ID NO: 31; (b) a HC-CDR2 comprising an amino acid sequence set forth in SEQ ID NO: 32; (c) a HC-CDR3 comprising an amino acid sequence set forth in SEQ ID NO: 33; (d) a LC-CDR1 comprising an amino acid sequence set forth in SEQ ID NO: 34; (e) a LC-CDR2 comprising an amino acid sequence set forth in SEQ ID NO: 35; and (f) a LC-CDR3 comprising an amino acid sequence set forth in SEQ ID NO: 36. In some embodiments, the CDRs are human CDRs. In some embodiments, the full-length anti-WTMC IgG1 antibody comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 95 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 96. In some embodiments, the full-length anti-WTMC IgG1 antibody comprises a V_{H} domain comprising SEQ ID NO: 95 and a V_{L} domain comprising SEQ ID NO: 96.

In some embodiments, the full-length anti-WTMC IgG1 antibody comprises: (a) a HC-CDR1 comprising an amino acid sequence set forth in SEQ ID NO: 37; (b) a HC-CDR2 comprising an amino acid sequence set forth in SEQ ID NO: 38; (c) a HC-CDR3 comprising an amino acid sequence set forth in SEQ ID NO: 39; (d) a LC-CDR1 comprising an amino acid sequence set forth in SEQ ID NO: 40; (e) a LC-CDR2 comprising an amino acid sequence set forth in SEQ ID NO: 41; and (f) a LC-CDR3 comprising an amino acid sequence set forth in SEQ ID NO: 42. In some embodiments, the CDRs are human CDRs. In some embodiments, the full-length anti-WTMC IgG1 antibody comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 97 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 98. In some embodiments, the full-length anti-WTMC IgG1 antibody comprises a V_{H} domain comprising SEQ ID NO: 97 and a V_{L} domain comprising SEQ ID NO: 98.

In some embodiments, the full-length anti-WTMC IgG1 antibody comprises: (a) a HC-CDR1 comprising an amino acid sequence set forth in SEQ ID NO: 43; (b) a HC-CDR2 comprising an amino acid sequence set forth in SEQ ID NO: 44; (c) a HC-CDR3 comprising an amino acid sequence set forth in SEQ ID NO: 45; (d) a LC-CDR1 comprising an amino acid sequence set forth in SEQ ID NO: 46; (e) a LC-CDR2 comprising an amino acid sequence set forth in SEQ ID NO: 47; and (f) a LC-CDR3 comprising an amino acid sequence set forth in SEQ ID NO: 48. In some embodiments, the CDRs are human CDRs. In some embodiments, the full-length anti-WTMC IgG1 antibody comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 99 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 100. In some embodiments, the full-length anti-WTMC IgG1 antibody comprises a V_{H} domain comprising SEQ ID NO: 99 and a V_{L} domain comprising SEQ ID NO: 100.

In some embodiments, the full-length anti-WTMC IgG1 antibody comprises: (a) a HC-CDR1 comprising an amino acid sequence set forth in SEQ ID NO: 49; (b) a HC-CDR2 comprising an amino acid sequence set forth in SEQ ID NO: 50; (c) a HC-CDR3 comprising an amino acid sequence set forth in SEQ ID NO: 51; (d) a LC-CDR1 comprising an amino acid sequence set forth in SEQ ID NO: 52; (e) a LC-CDR2 comprising an amino acid sequence set forth in SEQ ID NO: 53; and (f) a LC-CDR3 comprising an amino acid sequence set forth in SEQ ID NO: 54. In some embodiments, the CDRs are human CDRs. In some embodiments, the full-length anti-WTMC IgG1 antibody comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 101 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 102. In some embodiments, the full-length anti-WTMC IgG1 antibody comprises a V_{H} domain comprising SEQ ID NO: 101 and a V_{L} domain comprising SEQ ID NO: 102.

In some embodiments, the full-length anti-WTMC IgG1 antibody comprises: (a) a HC-CDR1 comprising an amino acid sequence set forth in SEQ ID NO: 55; (b) a HC-CDR2 comprising an amino acid sequence set forth in SEQ ID NO: 56; (c) a HC-CDR3 comprising an amino acid sequence set forth in SEQ ID NO: 57; (d) a LC-CDR1 comprising an amino acid sequence set forth in SEQ ID NO: 58; (e) a LC-CDR2 comprising an amino acid sequence set forth in SEQ ID NO: 59; and (f) a LC-CDR3 comprising an amino acid sequence set forth in SEQ ID NO: 60. In some embodiments, the CDRs are human CDRs. In some embodiments, the full-length anti-WTMC IgG1 antibody comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 103 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 104. In some embodiments, the full-length anti-WTMC IgG1 antibody comprises a V_{H} domain comprising SEQ ID NO: 103 and a V_{L} domain comprising SEQ ID NO: 104.

In some embodiments, the full-length anti-WTMC IgG1 antibody comprises: (a) a HC-CDR1 comprising an amino acid sequence set forth in SEQ ID NO: 61; (b) a HC-CDR2 comprising an amino acid sequence set forth in SEQ ID NO: 62; (c) a HC-CDR3 comprising an amino acid sequence set forth in SEQ ID NO: 63; (d) a LC-CDR1 comprising an amino acid sequence set forth in SEQ ID NO: 64; (e) a LC-CDR2 comprising an amino acid sequence set forth in SEQ ID NO: 65; and (f) a LC-CDR3 comprising an amino acid sequence set forth in SEQ ID NO: 66. In some embodiments, the CDRs are human CDRs. In some embodiments, the full-length anti-WTMC IgG1 antibody comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 105 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 106. In some embodiments, the full-length anti-WTMC IgG1 antibody comprises a V_{H} domain comprising SEQ ID NO: 105 and a V_{L} domain comprising SEQ ID NO: 106.

In some embodiments, the full-length anti-WTMC IgG1 antibody comprises: (a) a HC-CDR1 comprising an amino acid sequence set forth in SEQ ID NO: 67; (b) a HC-CDR2 comprising an amino acid sequence set forth in SEQ ID NO: 68; (c) a HC-CDR3 comprising an amino acid sequence set forth in SEQ ID NO: 69; (d) a LC-CDR1 comprising an amino acid sequence set forth in SEQ ID NO: 70; (e) a LC-CDR2 comprising an amino acid sequence set forth in SEQ ID NO: 71; and (f) a LC-CDR3 comprising an amino acid sequence set forth in SEQ ID NO: 72. In some embodiments, the CDRs are human CDRs. In some embodiments, the full-length anti-WTMC IgG1 antibody comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 107 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 108. In some embodiments, the full-length anti-WTMC IgG1 antibody comprises a V_{H} domain comprising SEQ ID NO: 107 and a V_{L} domain comprising SEQ ID NO: 108.

In some embodiments, the full-length anti-WTMC IgG1 antibody comprises: (a) a HC-CDR1 comprising an amino acid sequence set forth in SEQ ID NO: 73; (b) a HC-CDR2 comprising an amino acid sequence set forth in SEQ ID NO: 74; (c) a HC-CDR3 comprising an amino acid sequence set forth in SEQ ID NO: 75; (d) a LC-CDR1 comprising an amino acid sequence set forth in SEQ ID NO: 76; (e) a LC-CDR2 comprising an amino acid sequence set forth in SEQ ID NO: 77; and (f) a LC-CDR3 comprising an amino acid sequence set forth in SEQ ID NO: 78. In some embodiments, the CDRs are human CDRs. In some embodiments, the full-length anti-WTMC IgG1 antibody comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 109 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 110. In some embodiments, the full-length anti-WTMC IgG1 antibody comprises a V_{H} domain comprising SEQ ID NO: 109 and a V_{L} domain comprising SEQ ID NO: 110.

In some embodiments, the full-length anti-WTMC IgG1 antibody comprises: (a) a HC-CDR1 comprising an amino acid sequence set forth in SEQ ID NO: 79; (b) a HC-CDR2 comprising an amino acid sequence set forth in SEQ ID NO: 80; (c) a HC-CDR3 comprising an amino acid sequence set forth in SEQ ID NO: 81; (d) a LC-CDR1 comprising an amino acid sequence set forth in SEQ ID NO: 82; (e) a LC-CDR2 comprising an amino acid sequence set forth in SEQ ID NO: 83; and (f) a LC-CDR3 comprising an amino acid sequence set forth in SEQ ID NO: 84. In some embodiments, the CDRs are human CDRs. In some embodiments, the full-length anti-WTMC IgG1 antibody comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 111 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 112. In some embodiments, the full-length anti-WTMC IgG1 antibody comprises a V_{H} domain comprising SEQ ID NO: 111 and a V_{L} domain comprising SEQ ID NO: 112.

In some embodiments, the full-length anti-WTMC antibody binds to a complex comprising a WT1 peptide (e.g., WT1-RMF) and an MHC class I protein with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the full-length anti-WTMC antibody binds to a complex comprising a WT1 peptide (e.g., WT1-RMF) and an MHC class I protein with a K_{d} between about 1 pM to about 250 pM (such as about any of 1, 10, 25, 50, 75, 100, 150, 200, or 250 pM, including any ranges between these values).

### Multispecific Anti-WTMC Molecules

The anti-WTMC constructs in some embodiments comprise a multispecific anti-WTMC molecule comprising an anti-WTMC antibody moiety and a second binding moiety (such as a second antigen-binding moiety). In some embodiments, the multi-specific anti-WTMC molecule comprises an anti-WTMC antibody moiety and a second antigen-binding moiety.

Multi-specific molecules are molecules that have binding specificities for at least two different antigens or epitopes (e.g., bispecific antibodies have binding specificities for two antigens or epitopes). Multi-specific molecules with more than two valencies and/or specificities are also contemplated. For example, trispecific antibodies can be prepared. Tutt et al. J. Immunol. 147: 60 (1991). It is to be appreciated that one of skill in the art could select appropriate features of individual multi-specific molecules described herein to combine with one another to form a multi-specific anti-WTMC molecule of the present application.

Thus, for example, in some embodiments, there is provided a multi-specific (e.g., bispecific) anti-WTMC molecule comprising a) any one of the anti-WTMC antibody moieties described herein that specifically binds to a complex comprising a WT1 peptide (e.g., WT1-RMF) and an MHC class I protein, and b) a second binding moiety (such as an antigen-binding moiety). In some embodiments, the second binding moiety specifically binds to a complex comprising a different WT1 peptide (e.g., a peptide other than WT1-RMF) bound to the MHC class I protein. In some embodiments, the second scFv specifically binds to a complex comprising the WT1 peptide (e.g., WT1-RMF) bound to a different MHC class I protein. In some embodiments, the second binding moiety specifically binds to a different epitope on the complex comprising the WT1 peptide (e.g., WT1-RMF) and the MHC class I protein. In some embodiments, the second binding moiety specifically binds to a different antigen. In some embodiments, the second binding moiety specifically binds to an antigen on the surface of a cell, such as a cytotoxic cell. In some embodiments, the second binding moiety specifically binds to an antigen on the surface of a lymphocyte, such as a T cell, an NK cell, a neutrophil, a monocyte, a macrophage, or a dendritic cell. In some embodiments, the second binding moiety specifically binds to an effector T cell, such as a cytotoxic T cell (also known as cytotoxic T lymphocyte (CTL) or T killer cell).

In some embodiments, there is provided a multispecific anti-WTMC molecule comprising a) an anti-WTMC antibody moiety, and b) a second antigen-binding moiety that binds specifically to CD3. In some embodiments, the second antigen-binding moiety specifically binds to CD3ε. In some embodiments, the second antigen-binding moiety specifically binds to an agonistic epitope of CD3ε. The term “agonistic epitope”, as used herein, means (a) an epitope that, upon binding of the multi-specific molecule, optionally upon binding of several multi-specific molecules on the same cell, allows said multi-specific molecules to activate T cell receptor signaling and induce T cell activation, and/or (b) an epitope that is solely composed of amino acid residues of the epsilon chain of CD3 and is accessible for binding by the multi-specific molecule, when presented in its natural context on T cells (i.e. surrounded by the TCR, the CD3γ chain, etc.), and/or (c) an epitope that, upon binding of the multi-specific molecule, does not lead to stabilization of the spatial position of CD3ε relative to CD3γ.

In some embodiments, there is provided a multispecific anti-WTMC molecule comprising a) an anti-WTMC antibody moiety, and b) a second antigen-binding moiety that binds specifically to an antigen on the surface of an effector cell, including for example CD3γ, CD3δ, CD3ε, CD3ζ, CD28, CD16a, CD56, CD68, and GDS2D.

In some embodiments, there is provided a multispecific anti-WTMC molecule comprising a) an anti-WTMC antibody moiety, and b) a second antigen-binding moiety that binds specifically to a component of the complement system, such as C1q. C1q is a subunit of the C1 enzyme complex that activates the serum complement system.

In some embodiments, the second antigen-binding moiety specifically binds to an Fc receptor. In some embodiments, the second antigen-binding moiety specifically binds to an Fcγ receptor (FcγR). The FcγR may be an FcγRIII present on the surface of natural killer (NK) cells or one of FcγRI, FcγRIIA, FcγRIIBI, FcγRIIB2, and FcγRIIIB present on the surface of macrophages, monocytes, neutrophils and/or dendritic cells. In some embodiments, the second antigen-binding moiety is an Fc region or functional fragment thereof. A “functional fragment” as used in this context refers to a fragment of an antibody Fc region that is still capable of binding to an FcR, in particular to an FcγR, with sufficient specificity and affinity to allow an FcγR bearing effector cell, in particular a macrophage, a monocyte, a neutrophil and/or a dendritic cell, to kill the target cell by cytotoxic lysis or phagocytosis. A functional Fc fragment is capable of competitively inhibiting the binding of the original, full-length Fc portion to an FcR such as the activating FcγRI. In some embodiments, a functional Fc fragment retains at least 30%, 40%, 50%, 60%, 70%, 80%, 90% or 95% of its affinity to an activating FcγR. In some embodiments, the Fc region or functional fragment thereof is an enhanced Fc region or functional fragment thereof. The term “enhanced Fc region”, as used herein, refers to an Fc region that is modified to enhance Fc receptor-mediated effector-functions, in particular antibody-dependent cell-mediated cytotoxicity (ADCC), complement-dependent cytotoxicity (CDC), and antibody-mediated phagocytosis. This can be achieved as known in the art, for example by altering the Fc region in a way that leads to an increased affinity for an activating receptor (e.g., FcγRIIIA (CD16A) expressed on natural killer (NK) cells) and/or a decreased binding to an inhibitory receptor (e.g., FcγRIIB1/B2 (CD32B)). In yet other embodiments, the second antigen-binding moiety is an antibody or antigen-binding fragment thereof that specifically binds to an FcR, in particular to an FcγR, with sufficient specificity and affinity to allow an FcγR bearing effector cell, in particular a macrophage, a monocyte, a neutrophil and/or a dendritic cell, to kill the target cell by cytotoxic lysis or phagocytosis.

In some embodiments, the multispecific anti-WTMC molecule allows killing of WTMC-presenting target cells and/or can effectively redirect CTLs to lyse WTMC-presenting target cells. In some embodiments, the multi-specific (e.g., bispecific) anti-WTMC molecule of the present application shows an in vitro EC₅₀ ranging from 10 to 500 ng/mL and is able to induce redirected lysis of about 50% of the target cells through CTLs at a ratio of CTLs to target cells of from about 1:1 to about 50:1 (such as from about 1:1 to about 15:1, or from about 2:1 to about 10:1).

In some embodiments, the multispecific (e.g., bispecific) anti-WTMC molecule is capable of cross-linking a stimulated or unstimulated CTL and the target cell in such a way that the target cell is lysed. This offers the advantage that no generation of target-specific T cell clones or common antigen presentation by dendritic cells is required for the multispecific anti-WTMC molecule to exert its desired activity. In some embodiments, the multispecific anti-WTMC molecule of the present application is capable of redirecting CTLs to lyse the target cells in the absence of other activating signals. In some embodiments, the second antigen-binding moiety of the multispecific anti-WTMC molecule specifically binds to CD3 (e.g., specifically binds to CD3ε), and signaling through CD28 and/or IL-2 is not required for redirecting CTLs to lyse the target cells.

Methods for measuring the preference of the multispecific anti-WTMC molecule to simultaneously bind to two antigens (e.g., antigens on two different cells) are within the normal capabilities of a person skilled in the art. For example, when the second binding moiety specifically binds to CD3, the multi-specific anti-WTMC molecule may be contacted with a mixture of CD3⁺/WT1⁻ cells and CD3⁻/WT1⁺ cells. The number of multispecific anti-WTMC molecule-positive single cells and the number of cells cross-linked by multispecific anti-WTMC molecules may then be assessed by microscopy or fluorescence-activated cell sorting (FACS) as known in the art.

For example, in some embodiments, there is provided a multi-specific anti-WTMC molecule comprising a) any one of the anti-WTMC antibody moieties described herein that specifically binds to a complex comprising a WT1 peptide (e.g., WT1-RMF) and an MHC class I protein, and b) a second antigen-binding moiety. In some embodiments, the WT1 peptide (e.g., WT1-RMF) comprises SEQ ID NO: 113. In some embodiments, the antibody moiety specifically recognizes amino acid residues FPNA (SEQ ID NO: 349) of SEQ ID NO: 113. In some embodiments, the MHC class I protein is HLA-A02. In some embodiments, the MHC class I protein is HLA-A*02:01. In some embodiments, the second antigen-binding moiety specifically binds to a complex comprising a different WT1 peptide (e.g., a peptide other than WT1-RMF) bound to the MHC class I protein. In some embodiments, the second antigen-binding moiety specifically binds to a complex comprising the WT1 peptide (e.g., WT1-RMF) bound to a different MHC class I protein. In some embodiments, the second antigen-binding moiety specifically binds to a different epitope on the complex comprising the WT1 peptide (e.g., WT1-RMF) and the MHC class I protein. In some embodiments, the second antigen-binding moiety specifically binds to another antigen. In some embodiments, the second antigen-binding moiety specifically binds to an antigen on the surface of a cell, such as a WTMC complex-presenting cell. In some embodiments, the second antigen-binding moiety specifically binds to an antigen on the surface of a cell that does not express a WTMC complex. In some embodiments, the second antigen-binding moiety specifically binds to an antigen on the surface of a cytotoxic cell. In some embodiments, the second antigen-binding moiety specifically binds to an antigen on the surface of a lymphocyte, such as a T cell, an NK cell, a neutrophil, a monocyte, a macrophage, or a dendritic cell. In some embodiments, the second antigen-binding moiety specifically binds to an antigen on the surface of an effector T cell, such as a cytotoxic T cell. In some embodiments, the second antigen-binding moiety specifically binds to an antigen on the surface of an effector cell, including for example CD3γ, CD3δ, CD3ε, CD3ζ, CD28, CD16a, CD56, CD68, and GDS2D. In some embodiments, the anti-WTMC antibody moiety is human, humanized, or semi-synthetic. In some embodiments, the second antigen-binding moiety is an antibody moiety. In some embodiments, the second antigen-binding moiety is a human, humanized, or semi-synthetic antibody moiety. In some embodiments, the multispecific anti-WTMC molecule further comprises at least one (such as at least about any of 2, 3, 4, 5, or more) additional antigen-binding moieties.

In some embodiments, there is provided a multispecific anti-WTMC molecule comprising a) an anti-WTMC antibody moiety as described in the present application, and b) a second antigen-binding moiety. In some embodiments, the second antigen-binding moiety specifically binds CD3. In some embodiments, the second antigen-binding moiety specifically binds CD3ε. In some embodiments, the second antigen-binding moiety is a CD3ε antibody that specifically binds CD3ε. For additional description of CD3ε antibodies, see, for example, U.S. Ser. No. 10/968,276, the contents of which are herein incorporated by reference in its entirety.

In some embodiments, the second antigen-binding moiety comprises a full-length antibody, a Fab, a Fab′, a F(ab′)₂, an Fv, or a scFv.

In some embodiments, the multispecific anti-WTMC construct is, for example, a bispecific antibody, a diabody (Db), a single-chain diabody (scDb), a tandem scDb (Tandab), a linear dimeric scDb (LD-scDb), a circular dimeric scDb (CD-scDb), a di-diabody, a tandem scFv, a tandem di-scFv, a tandem tri-scFv, a tri(a)body, a bispecific Fab2, a di-miniantibody, a tetrabody, an scFv-Fc-scFv fusion, a dual-affinity retargeting (DART) antibody, a dual variable domain (DVD) antibody, an IgG-scFab, an scFab-ds-scFv, an Fv2-Fc, an IgG-scFv fusion, a dock and lock (DNL) antibody, a knob-into-hole (KiH) antibody (bispecific IgG prepared by the KiH technology), a DuoBody (bispecific IgG prepared by the Duobody technology), a heteromultimeric antibody, or a heteroconjugate antibody. In some embodiments, the multispecific anti-WTMC molecule is a tandem scFv (e.g., a tandem di-scFv). It is to be appreciated that one of ordinary skill in the art could select appropriate features of various multispecific constructs known in the art and combine them with one another to form a further multispecific anti-WTMC construct within the scope of this disclosure.

Suitable methods for making multispecific constructs (e.g., bispecific antibodies) are well known in the art. For example, the production of bispecific antibodies can be based on the co-expression of two immunoglobulin heavy-chain/light-chain pairs, where the two pairs each have different specificities, and upon association result in a heterodimeric antibody (see, e.g., Milstein and Cuello, Nature, 305: 537-539 (1983); WO 93/08829, and Traunecker et al., EMBO J. 10: 3655 (1991)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829 and in Traunecker et al., EMBO, 10: 3655-3659 (1991). Alternatively, the combining of heavy and light chains can be directed by taking advantage of species-restricted pairing (see, e.g., Lindhofer et al., J. Immunol., 155:219-225 (1995)) and the pairing of heavy chains can be directed by use of “knob-into hole” engineering of CH3 domains (see, e.g., U.S. Pat. No. 5,731,168; Ridgway et al., Protein Eng., 9(7):617-621 (1996)). Multispecific antibodies may also be made by engineering electrostatic steering effects for making antibody Fc-heterodimeric molecules (see, e.g., WO 2009/089004A1). In yet another method, stable bispecific antibodies can be generated by controlled Fab-arm exchange, where two parental antibodies having distinct antigen specificity and matched point mutations in the CH3 domains are mixed in reducing condition to allow for separation, reassembly, and reoxidation to form highly pure bispecific antibodies. Labrigin et al., Proc. Natl. Acad. Sci., 110(13):5145-5150 (2013). Such antibodies, comprising a mixture of heavy-chain/light-chain pairs, are also referred to herein as “heteromultimeric antibodies.”

Antibodies or antigen-binding fragments thereof having different specificities can also be chemically cross-linked to generate multispecific heteroconjugate antibodies. For example, two F(ab′)2 molecules, each having specificity for a different antigen, can be chemically linked. Pullarkat et al., Trends Biotechnol., 48:9-21 (1999). Such antibodies have, for example, been proposed to target immune-system cells to unwanted cells (U.S. Pat. No. 4,676,980), and for treatment of HIV infection. WO 91/00360; WO 92/200373; EP 03089. It is contemplated that the antibodies can be prepared in vitro using known methods in synthetic protein chemistry, including those involving crosslinking agents. For example, immunotoxins can be constructed using a disulfide-exchange reaction or by forming a thioether bond. Examples of suitable reagents for this purpose include iminothiolate and methyl-4-mercaptobutyrimidate and those disclosed, for example, in U.S. Pat. No. 4,676,980.

In some embodiments, multispecific anti-WTMC constructs can be prepared using recombinant DNA techniques. For example, a bispecific antibody can be engineered by fusing two scFvs, such as by fusing them through a peptide linker, resulting in a tandem scFv (such as a tandem di-scFv). The terms “anti-WTMC tandem di-scFv” and “bispecific anti-WTMC antibody” are used interchangeably herein. In some embodiments, the tandem scFv comprises an anti-CD3 scFv to an scFv comprising an anti-WTMC binding moiety described herein, resulting in the redirection of T cells to target cells that express (such as overexpress) the target. Additional details regarding the construction and expression of tandem scFvs are provided in, e.g., Mack et al., Proc. Natl. Acad. Sci., 92:7021-7025 (1995); Brischwein et al., Mol. Immunol., 43(8):1129-1143 (2006). Additional details regarding tandem scFvs of the present disclosure are provided elsewhere herein.

By shortening the length of a peptide linker between two variable domains, the variable domains can be prevented from self-assembling and forced to pair with domains on a second polypeptide, resulting in a compact bispecific antibody called a diabody (Db). Holliger et al., Proc. Natl. Acad. Sci., 90:6444-6448 (1993). The two polypeptides of a Db each comprise a V_{H} connected to a V_{L} by a linker which is too short to allow pairing between the two domains on the same chain. Accordingly, the V_{H} and V_{L} domains of one polypeptide are forced to pair with the complementary V_{L} and V_{H} domains of another polypeptide, thereby forming two antigen-binding sites. In a modification of this format, the two polypeptides are linked by another peptide linker, resulting in a single chain diabody (scDb). In yet another modification of the Db format, dual-affinity retargeting (DART) bispecific antibodies can be generated by introducing a disulfide linkage between cysteine residues at the C-terminus of each polypeptide, optionally including domains prior to the C-terminal cysteine residues that drive assembly of the desired heterodimeric structure. Veri et al., Arthritis Rheum., 62(7):1933-1943 (2010). Dual-variable-domain immunoglobulins (DVD-Ig™), in which the target-binding variable domains of two monoclonal antibodies are combined via naturally occurring linkers to yield a tetravalent, bispecific antibody, are also known in the art. Gu and Ghayur, Methods Enzymol., 502:25-41 (2012). In yet another format, Dock and Lock (DNL), bispecific antibodies are prepared by taking advantage of the dimerization of a peptide (DDD2) derived from the regulatory subunit of human cAMP-dependent protein kinase (PKA) with a peptide (AD2) derived from the anchoring domains of human A kinase anchor proteins (AKAPs). Rossi et al., Proc. Natl. Acad. Sci., 103:6841-6846 (2006).

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). This method can also be utilized for the production of antibody homodimers.

Tandem scFv Constructs

In some embodiments, the multispecific anti-WTMC construct is a tandem scFv construct (“anti-WTMC tandem scFv”) comprising a first scFv that comprises an anti-WTMC antibody moiety (such as described herein) and a second scFv that binds to a second target. In some embodiments, the tandem scFv is a di-scFv (comprising two scFv) or a tandem tri-scFv (comprising three scFv). In some embodiments, the anti-WTMC tandem scFv further comprises at least 3, 4, 5, 6, 7, 8, 9, 10, or more scFvs. In some embodiments, the second scFv specifically binds to a WTMC complex (such as an epitope that does not overlap the epitope bound by the anti-WTMC antibody moiety of the first scFv. In some embodiments, the second scFv specifically binds to another antigen (i.e., an antigen other than the target). In some embodiments, the second scFv binds to a target ligand other than a WTMC complex. In some embodiments, the second scFv specifically binds to an antigen on the surface of a cell, such as a cell that expresses a WTMC complex (e.g., a cancer cell). In some embodiments, the second scFv specifically binds to an antigen on the surface of a cell that does not express a WTMC complex. In some embodiments, the second scFv specifically binds to an antigen on the surface of a cytotoxic cell. In some embodiments, the second scFv specifically binds to an antigen on the surface of a lymphocyte, such as a T cell, an NK cell, a neutrophil, a monocyte, a macrophage, or a dendritic cell. In some embodiments, the second scFv specifically binds to an antigen on the surface of an effector T cell, such as a cytotoxic T cell. In some embodiments, the second scFv specifically binds to an antigen on the surface of an effector cell, including for example CD3γ, CD3δ, CD3ε, CD3ζ, CD28, CD16a, CD56, CD68, and GDS2D. In some embodiments, the first scFv and/or the second scFv is human, humanized, or semi-synthetic.

In some embodiments, there is provided a tandem scFv multi-specific (e.g., bispecific) anti-WTMC antibody comprising a) a first scFv that specifically binds to a complex comprising a WT1 peptide (e.g., WT1-RMF) and an MHC class I protein, and b) a second scFv. In some embodiments, the WT1 peptide (e.g., WT1-RMF) comprises SEQ ID NO: 113. In some embodiments, the first scFv specifically recognizes amino acid residues FPNA (SEQ ID NO: 349) of SEQ ID NO: 113. In some embodiments, the MHC class I protein is HLA-A02. In some embodiments, the MHC class I protein is HLA-A*02:01. In some embodiments, the second scFv specifically binds to a complex comprising a different WT1 peptide bound to the MHC class I protein. In some embodiments, the second scFv specifically binds to a complex comprising the WT1 peptide bound to a different MHC class I protein. In some embodiments, the second scFv specifically binds to a different epitope on the complex comprising the WT1 peptide and the MHC class I protein. In some embodiments, the second scFv specifically binds to another antigen. In some embodiments, the second scFv specifically binds to an antigen on the surface of a cell, such as an WTMC-presenting cell. In some embodiments, the second scFv specifically binds to an antigen on the surface of a cell that does not express WT1. In some embodiments, the second scFv specifically binds to an antigen on the surface of a cytotoxic cell. In some embodiments, the second scFv specifically binds to an antigen on the surface of a lymphocyte, such as a T cell, an NK cell, a neutrophil, a monocyte, a macrophage, or a dendritic cell. In some embodiments, the second scFv specifically binds to an antigen on the surface of an effector T cell, such as a cytotoxic T cell. In some embodiments, the second scFv specifically binds to an antigen on the surface of an effector cell, including for example CD3γ, CD3δ, CD3ε, CD3ζ, CD28, CD16a, CD56, CD68, and GDS2D. In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic. In some embodiments, the tandem scFv multi-specific anti-WTMC antibody further comprises at least one (such as at least about any of 2, 3, 4, 5, or more) additional scFv. In some embodiments, the anti-WTMC antibody moiety cross-reacts with at least one (such as at least any of 2, 3, 4, 5, or 6) complex comprising the MHC class I protein and a variant of the WT1 peptide having one amino acid substitution (such as a conservative amino acid substitution). In some embodiments, the anti-WTMC antibody moiety cross-reacts with at least one (such as at least any of 2, 3, 4, or 5) complex comprising the WT1 peptide (e.g., WT1-RMF) and a different subtype of the MHC class I protein.

In some embodiments, there is provided a tandem scFv multi-specific (e.g., bispecific) anti-WTMC antibody comprising a) a first scFv that specifically binds to a WTMC as described in the present application; and b) a second scFv.

In some embodiments, there is provided a tandem scFv multi-specific (e.g., bispecific) anti-WTMC antibody comprising a) a first scFv that specifically binds to a WTMC, and b) a second scFv, wherein the tandem scFv multispecific anti-WTMC antibody is a tandem di-scFv or a tandem tri-scFv. In some embodiments, the tandem scFv multispecific anti-WTMC antibody is a tandem di-scFv. In some embodiments, the tandem scFv multispecific anti-WTMC antibody is a bispecific T-cell engager.

For example, in some embodiments, there is provided a tandem di-scFv bispecific anti-WTMC antibody comprising a) a first scFv that specifically binds to a complex comprising a WT1 peptide (e.g., WT1-RMF) and an MHC class I protein, and b) a second scFv that specifically binds to an antigen on the surface of a T cell. In some embodiments, the WT1 peptide (e.g., WT1-RMF) comprises SEQ ID NO: 113. In some embodiments, the first scFv specifically recognizes amino acid residues FPNA (SEQ ID NO: 349) of SEQ ID NO: 113. In some embodiments, the MHC class I protein is HLA-A02. In some embodiments, the MHC class I protein is HLA-A*02:01. In some embodiments, the second scFv specifically binds to an antigen on the surface of an effector T cell, such as a cytotoxic T cell. In some embodiments, the second scFv specifically binds to an antigen selected, for example, from the group consisting of CD3, CD3γ, CD3δ, CD3ε, CD3ζ, CD28, OX40, GITR, CD137, CD27, CD40L, and HVEM. In some embodiments, the second scFv specifically binds to an agonistic epitope on an antigen on the surface of a T cell, wherein the binding of the second scFv to the antigen enhances T cell activation. In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic.

In some embodiments, there is provided a tandem di-scFv bispecific anti-WTMC antibody comprising a) a first scFv that specifically binds to a WTMC as described in the present application; and b) a second scFv that specifically binds to an antigen on the surface of a T cell.

In some embodiments, there is provided a tandem di-scFv bispecific anti-WTMC antibody comprising a) a first scFv that specifically binds to a complex comprising a WT1 peptide (e.g., WT1-RMF) and an MHC class I protein, and b) a second scFv that specifically binds to CD3ε. In some embodiments, the WT1 peptide (e.g., WT1-RMF) comprises SEQ ID NO: 113. In some embodiments, the first scFv specifically recognizes amino acid residues FPNA (SEQ ID NO: 349) of SEQ ID NO: 113. In some embodiments, the MHC class I protein is HLA-A02. In some embodiments, the MHC class I protein is HLA-A*02:01. In some embodiments, the first scFv is fused to the second scFv through linkage with a peptide linker. In some embodiments, the peptide linker is between about 5 to about 20 (such as about any of 5, 10, 15, or 20, including any ranges between these values) amino acids in length. In some embodiments, the peptide linker comprises (and in some embodiments consists of) the amino acid sequence SRGGGGSGGGGSGGGGSLEMA (SEQ ID NO: 249). In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic.

In some embodiments, there is provided a tandem di-scFv bispecific anti-WTMC antibody comprising a) a first scFv that specifically binds to a complex comprising a WT1 peptide (e.g., WT1-RMF) comprising SEQ ID NO: 113 and HLA-A*02:01, and b) a second scFv that specifically binds to CD3ε. In some embodiments, the first scFv specifically recognizes amino acid residues FPNA (SEQ ID NO: 349) of SEQ ID NO: 113. In some embodiments, the first scFv is fused to the second scFv through linkage with a peptide linker. In some embodiments, the peptide linker is between about 5 to about 20 (such as about any of 5, 10, 15, or 20, including any ranges between these values) amino acids in length. In some embodiments, the peptide linker comprises (and in some embodiments consists of) the amino acid sequence of SEQ ID NO: 249. In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic.

In some embodiments, there is provided a tandem di-scFv bispecific anti-WTMC antibody comprising a) a first scFv that specifically binds to a WTMC as described in the present application; and b) a second scFv that specifically binds to CD3 g. In some embodiments, the first scFv is fused to the second scFv through linkage with a peptide linker. In some embodiments, the peptide linker is between about 5 to about 20 (such as about any of 5, 10, 15, or 20, including any ranges between these values) amino acids in length. In some embodiments, the peptide linker comprises (and in some embodiments consists of) the amino acid sequence of SEQ ID NO: 249. In some embodiments, the first scFv is human, humanized, or semi-synthetic. In some embodiments, the second scFv is human, humanized, or semi-synthetic. In some embodiments, both the first scFv and the second scFv are human, humanized, or semi-synthetic.

In some embodiments, there is provided a tandem di-scFv bispecific anti-WTMC antibody comprising a) a first scFv that specifically binds to a WTMC as described in the present application; and b) a second scFv that specifically binds to CD3.

In some embodiments, the tandem di-scFv bispecific anti-WTMC antibody comprises (i) a first scFv comprising (a) a HC-CDR1 comprising an amino acid sequence set forth in SEQ ID NO: 1; (b) a HC-CDR2 comprising an amino acid sequence set forth in SEQ ID NO: 2; (c) a HC-CDR3 comprising an amino acid sequence set forth in SEQ ID NO: 3; (d) a LC-CDR1 comprising an amino acid sequence set forth in SEQ ID NO: 4; (e) a LC-CDR2 comprising an amino acid sequence set forth in SEQ ID NO: 5; and (f) a LC-CDR3 comprising an amino acid sequence set forth in SEQ ID NO: 6; and (ii) a second scFv that specifically binds to CD3. In some embodiments, the tandem di-scFv bispecific anti-WTMC antibody comprises a) a first scFv comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 85 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 86; and b) a second scFv that specifically binds to CD3. In some embodiments, the tandem di-scFv bispecific anti-WTMC antibody comprises a) a first scFv comprising a V_{H} domain comprising the amino acid sequence of SEQ ID NO: 85 and a V_{L} domain comprising the amino acid sequence of SEQ ID NO: 86; and b) a second scFv that specifically binds to CD3.

In some embodiments, the tandem di-scFv bispecific anti-WTMC antibody comprises (i) a first scFv comprising (a) a HC-CDR1 comprising an amino acid sequence set forth in SEQ ID NO: 7; (b) a HC-CDR2 comprising an amino acid sequence set forth in SEQ ID NO: 8; (c) a HC-CDR3 comprising an amino acid sequence set forth in SEQ ID NO: 9; (d) a LC-CDR1 comprising an amino acid sequence set forth in SEQ ID NO: 10; (e) a LC-CDR2 comprising an amino acid sequence set forth in SEQ ID NO: 11; and (f) a LC-CDR3 comprising an amino acid sequence set forth in SEQ ID NO: 12; and (ii) a second scFv that specifically binds to CD3. In some embodiments, the tandem di-scFv bispecific anti-WTMC antibody comprises a) a first scFv comprising V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 87 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 88; and b) a second scFv that specifically binds to CD3. In some embodiments, the tandem di-scFv bispecific anti-WTMC antibody comprises a) a first scFv comprising a V_{H} domain comprising the amino acid sequence of SEQ ID NO: 87 and a V_{L} domain comprising the amino acid sequence of SEQ ID NO: 88; and b) a second scFv that specifically binds to CD3.

In some embodiments, the tandem di-scFv bispecific anti-WTMC antibody comprises (i) a first scFv comprising (a) a HC-CDR1 comprising an amino acid sequence set forth in SEQ ID NO: 13; (b) a HC-CDR2 comprising an amino acid sequence set forth in SEQ ID NO: 14; (c) a HC-CDR3 comprising an amino acid sequence set forth in SEQ ID NO: 15; (d) a LC-CDR1 comprising an amino acid sequence set forth in SEQ ID NO: 16; (e) a LC-CDR2 comprising an amino acid sequence set forth in SEQ ID NO: 17; and (f) a LC-CDR3 comprising an amino acid sequence set forth in SEQ ID NO: 18; and (ii) a second scFv that specifically binds to CD3. In some embodiments, the tandem di-scFv bispecific anti-WTMC antibody comprises a) a first scFv comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 89 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 90; and b) a second scFv that specifically binds to CD3. In some embodiments, the tandem di-scFv bispecific anti-WTMC antibody comprises a) a first scFv comprising a V_{H} domain comprising the amino acid sequence of SEQ ID NO: 89 and a V_{L} domain comprising the amino acid sequence of SEQ ID NO: 90; and b) a second scFv that specifically binds to CD3.

In some embodiments, the tandem di-scFv bispecific anti-WTMC antibody comprises (i) a first scFv comprising (a) a HC-CDR1 comprising an amino acid sequence set forth in SEQ ID NO: 19; (b) a HC-CDR2 comprising an amino acid sequence set forth in SEQ ID NO: 20; (c) a HC-CDR3 comprising an amino acid sequence set forth in SEQ ID NO: 21; (d) a LC-CDR1 comprising an amino acid sequence set forth in SEQ ID NO: 22; (e) a LC-CDR2 comprising an amino acid sequence set forth in SEQ ID NO: 23; and (f) a LC-CDR3 comprising an amino acid sequence set forth in SEQ ID NO: 24; and (ii) a second scFv that specifically binds to CD3. In some embodiments, the tandem di-scFv bispecific anti-WTMC antibody comprises a) a first scFv comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 91 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 92; and b) a second scFv that specifically binds to CD3. In some embodiments, the tandem di-scFv bispecific anti-WTMC antibody comprises a) a first scFv comprising a V_{H} domain comprising the amino acid sequence of SEQ ID NO: 91 and a V_{L} domain comprising the amino acid sequence of SEQ ID NO: 92; and b) a second scFv that specifically binds to CD3.

In some embodiments, the tandem di-scFv bispecific anti-WTMC antibody comprises (i) a first scFv comprising (a) a HC-CDR1 comprising an amino acid sequence set forth in SEQ ID NO: 25; (b) a HC-CDR2 comprising an amino acid sequence set forth in SEQ ID NO: 26; (c) a HC-CDR3 comprising an amino acid sequence set forth in SEQ ID NO: 27; (d) a LC-CDR1 comprising an amino acid sequence set forth in SEQ ID NO: 28; (e) a LC-CDR2 comprising an amino acid sequence set forth in SEQ ID NO: 29; and (f) a LC-CDR3 comprising an amino acid sequence set forth in SEQ ID NO: 30; and (ii) a second scFv that specifically binds to CD3. In some embodiments, the tandem di-scFv bispecific anti-WTMC antibody comprises a) a first scFv comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 93 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 94; and b) a second scFv that specifically binds to CD3. In some embodiments, the tandem di-scFv bispecific anti-WTMC antibody comprises a) a first scFv comprising a V_{H} domain comprising the amino acid sequence of SEQ ID NO: 93 and a V_{L} domain comprising the amino acid sequence of SEQ ID NO: 94; and b) a second scFv that specifically binds to CD3.

In some embodiments, the tandem di-scFv bispecific anti-WTMC antibody comprises (i) a first scFv comprising (a) a HC-CDR1 comprising an amino acid sequence set forth in SEQ ID NO: 31; (b) a HC-CDR2 comprising an amino acid sequence set forth in SEQ ID NO: 32; (c) a HC-CDR3 comprising an amino acid sequence set forth in SEQ ID NO: 33; (d) a LC-CDR1 comprising an amino acid sequence set forth in SEQ ID NO: 34; (e) a LC-CDR2 comprising an amino acid sequence set forth in SEQ ID NO: 35; and (f) a LC-CDR3 comprising an amino acid sequence set forth in SEQ ID NO: 36; and (ii) a second scFv that specifically binds to CD3. In some embodiments, the tandem di-scFv bispecific anti-WTMC antibody comprises a) a first scFv comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 95 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 96; and b) a second scFv that specifically binds to CD3. In some embodiments, the tandem di-scFv bispecific anti-WTMC antibody comprises a) a first scFv comprising a V_{H} domain comprising the amino acid sequence of SEQ ID NO: 95 and a V_{L} domain comprising the amino acid sequence of SEQ ID NO: 96; and b) a second scFv that specifically binds to CD3.

In some embodiments, the tandem di-scFv bispecific anti-WTMC antibody comprises (i) a first scFv comprising (a) a HC-CDR1 comprising an amino acid sequence set forth in SEQ ID NO: 37; (b) a HC-CDR2 comprising an amino acid sequence set forth in SEQ ID NO: 38; (c) a HC-CDR3 comprising an amino acid sequence set forth in SEQ ID NO: 39; (d) a LC-CDR1 comprising an amino acid sequence set forth in SEQ ID NO: 40; (e) a LC-CDR2 comprising an amino acid sequence set forth in SEQ ID NO: 41; and (f) a LC-CDR3 comprising an amino acid sequence set forth in SEQ ID NO: 42; and (ii) a second scFv that specifically binds to CD3. In some embodiments, the tandem di-scFv bispecific anti-WTMC antibody comprises a) a first scFv comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 97 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 98; and b) a second scFv that specifically binds to CD3. In some embodiments, the tandem di-scFv bispecific anti-WTMC antibody comprises a) a first scFv comprising a V_{H} domain comprising the amino acid sequence of SEQ ID NO: 97 and a V_{L} domain comprising the amino acid sequence of SEQ ID NO: 98; and b) a second scFv that specifically binds to CD3.

In some embodiments, the tandem di-scFv bispecific anti-WTMC antibody comprises (i) a first scFv comprising (a) a HC-CDR1 comprising an amino acid sequence set forth in SEQ ID NO: 43; (b) a HC-CDR2 comprising an amino acid sequence set forth in SEQ ID NO: 44; (c) a HC-CDR3 comprising an amino acid sequence set forth in SEQ ID NO: 45; (d) a LC-CDR1 comprising an amino acid sequence set forth in SEQ ID NO: 46; (e) a LC-CDR2 comprising an amino acid sequence set forth in SEQ ID NO: 47; and (f) a LC-CDR3 comprising an amino acid sequence set forth in SEQ ID NO: 48; and (ii) a second scFv that specifically binds to CD3. In some embodiments, the tandem di-scFv bispecific anti-WTMC antibody comprises a) a first scFv comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 99 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 100; and b) a second scFv that specifically binds to CD3. In some embodiments, the tandem di-scFv bispecific anti-WTMC antibody comprises a) a first scFv comprising a V_{H} domain comprising the amino acid sequence of SEQ ID NO: 99 and a V_{L} domain comprising the amino acid sequence of SEQ ID NO: 100; and b) a second scFv that specifically binds to CD3.

In some embodiments, the tandem di-scFv bispecific anti-WTMC antibody comprises (i) a first scFv comprising (a) a HC-CDR1 comprising an amino acid sequence set forth in SEQ ID NO: 49; (b) a HC-CDR2 comprising an amino acid sequence set forth in SEQ ID NO: 50; (c) a HC-CDR3 comprising an amino acid sequence set forth in SEQ ID NO: 51; (d) a LC-CDR1 comprising an amino acid sequence set forth in SEQ ID NO: 52; (e) a LC-CDR2 comprising an amino acid sequence set forth in SEQ ID NO: 53; and (f) a LC-CDR3 comprising an amino acid sequence set forth in SEQ ID NO: 54; and (ii) a second scFv that specifically binds to CD3. In some embodiments, the tandem di-scFv bispecific anti-WTMC antibody comprises a) a first scFv comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 101 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 102; and b) a second scFv that specifically binds to CD3. In some embodiments, the tandem di-scFv bispecific anti-WTMC antibody comprises a) a first scFv comprising a V_{H} domain comprising the amino acid sequence of SEQ ID NO: 101 and a V_{L} domain comprising the amino acid sequence of SEQ ID NO: 102; and b) a second scFv that specifically binds to CD3.

In some embodiments, the tandem di-scFv bispecific anti-WTMC antibody comprises (i) a first scFv comprising (a) a HC-CDR1 comprising an amino acid sequence set forth in SEQ ID NO: 55; (b) a HC-CDR2 comprising an amino acid sequence set forth in SEQ ID NO: 56; (c) a HC-CDR3 comprising an amino acid sequence set forth in SEQ ID NO: 57; (d) a LC-CDR1 comprising an amino acid sequence set forth in SEQ ID NO: 58; (e) a LC-CDR2 comprising an amino acid sequence set forth in SEQ ID NO: 59; and (f) a LC-CDR3 comprising an amino acid sequence set forth in SEQ ID NO: 60; and (ii) a second scFv that specifically binds to CD3. In some embodiments, the tandem di-scFv bispecific anti-WTMC antibody comprises a) a first scFv comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 103 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 104; and b) a second scFv that specifically binds to CD3. In some embodiments, the tandem di-scFv bispecific anti-WTMC antibody comprises a) a first scFv comprising a V_{H} domain comprising the amino acid sequence of SEQ ID NO: 103 and a V_{L} domain comprising the amino acid sequence of SEQ ID NO: 104; and b) a second scFv that specifically binds to CD3.

In some embodiments, the tandem di-scFv bispecific anti-WTMC antibody comprises (i) a first scFv comprising (a) a HC-CDR1 comprising an amino acid sequence set forth in SEQ ID NO: 61; (b) a HC-CDR2 comprising an amino acid sequence set forth in SEQ ID NO: 62; (c) a HC-CDR3 comprising an amino acid sequence set forth in SEQ ID NO: 63; (d) a LC-CDR1 comprising an amino acid sequence set forth in SEQ ID NO: 64; (e) a LC-CDR2 comprising an amino acid sequence set forth in SEQ ID NO: 65; and (f) a LC-CDR3 comprising an amino acid sequence set forth in SEQ ID NO: 66; and (ii) a second scFv that specifically binds to CD3. In some embodiments, the tandem di-scFv bispecific anti-WTMC antibody comprises a) a first scFv comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 105 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 106; and b) a second scFv that specifically binds to CD3. In some embodiments, the tandem di-scFv bispecific anti-WTMC antibody comprises a) a first scFv comprising a V_{H} domain comprising the amino acid sequence of SEQ ID NO: 105 and a V_{L} domain comprising the amino acid sequence of SEQ ID NO: 106; and b) a second scFv that specifically binds to CD3.

In some embodiments, the tandem di-scFv bispecific anti-WTMC antibody comprises (i) a first scFv comprising (a) a HC-CDR1 comprising an amino acid sequence set forth in SEQ ID NO: 67; (b) a HC-CDR2 comprising an amino acid sequence set forth in SEQ ID NO: 68; (c) a HC-CDR3 comprising an amino acid sequence set forth in SEQ ID NO: 69; (d) a LC-CDR1 comprising an amino acid sequence set forth in SEQ ID NO: 70; (e) a LC-CDR2 comprising an amino acid sequence set forth in SEQ ID NO: 71; and (f) a LC-CDR3 comprising an amino acid sequence set forth in SEQ ID NO: 72; and (ii) a second scFv that specifically binds to CD3. In some embodiments, the tandem di-scFv bispecific anti-WTMC antibody comprises a) a first scFv comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 107 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 108; and b) a second scFv that specifically binds to CD3. In some embodiments, the tandem di-scFv bispecific anti-WTMC antibody comprises a) a first scFv comprising a V_{H} domain comprising the amino acid sequence of SEQ ID NO: 107 and a V_{L} domain comprising the amino acid sequence of SEQ ID NO: 108; and b) a second scFv that specifically binds to CD3.

In some embodiments, the tandem di-scFv bispecific anti-WTMC antibody comprises (i) a first scFv comprising (a) a HC-CDR1 comprising an amino acid sequence set forth in SEQ ID NO: 73; (b) a HC-CDR2 comprising an amino acid sequence set forth in SEQ ID NO: 74; (c) a HC-CDR3 comprising an amino acid sequence set forth in SEQ ID NO: 75; (d) a LC-CDR1 comprising an amino acid sequence set forth in SEQ ID NO: 76; (e) a LC-CDR2 comprising an amino acid sequence set forth in SEQ ID NO: 77; and (f) a LC-CDR3 comprising an amino acid sequence set forth in SEQ ID NO: 78; and (ii) a second scFv that specifically binds to CD3. In some embodiments, the tandem di-scFv bispecific anti-WTMC antibody comprises a) a first scFv comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 109 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 110; and b) a second scFv that specifically binds to CD3. In some embodiments, the tandem di-scFv bispecific anti-WTMC antibody comprises a) a first scFv comprising a V_{H} domain comprising the amino acid sequence of SEQ ID NO: 109 and a V_{L} domain comprising the amino acid sequence of SEQ ID NO: 110; and b) a second scFv that specifically binds to CD3.

In some embodiments, the tandem di-scFv bispecific anti-WTMC antibody comprises (i) a first scFv comprising (a) a HC-CDR1 comprising an amino acid sequence set forth in SEQ ID NO: 79; (b) a HC-CDR2 comprising an amino acid sequence set forth in SEQ ID NO: 80; (c) a HC-CDR3 comprising an amino acid sequence set forth in SEQ ID NO: 81; (d) a LC-CDR1 comprising an amino acid sequence set forth in SEQ ID NO: 82; (e) a LC-CDR2 comprising an amino acid sequence set forth in SEQ ID NO: 83; and (f) a LC-CDR3 comprising an amino acid sequence set forth in SEQ ID NO: 84; and (ii) a second scFv that specifically binds to CD3. In some embodiments, the tandem di-scFv bispecific anti-WTMC antibody comprises a) a first scFv comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 111 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 112; and b) a second scFv that specifically binds to CD3. In some embodiments, the tandem di-scFv bispecific anti-WTMC antibody comprises a) a first scFv comprising a V_{H} domain comprising the amino acid sequence of SEQ ID NO: 111 and a V_{L} domain comprising the amino acid sequence of SEQ ID NO: 112; and b) a second scFv that specifically binds to CD3.

In some embodiments, the tandem di-scFv bispecific anti-WTMC antibody binds to a complex comprising a WT1 peptide and an MHC class I protein with a K_{d} between about 0.1 pM to about 500 nM (such as about any of 0.1 pM, 1.0 pM, 10 pM, 50 pM, 100 pM, 500 pM, 1 nM, 10 nM, 50 nM, 100 nM, or 500 nM, including any ranges between these values). In some embodiments, the tandem di-scFv bispecific anti-WTMC antibody binds to a complex comprising a WT1 peptide and an MHC class I protein with a K_{d} between about 1 nM to about 500 nM (such as about any of 1, 10, 25, 50, 75, 100, 150, 200, 250, 300, 350, 400, 450, or 500 nM, including any ranges between these values).

In some embodiments, the multi-specific anti-WTMC molecule (such as di-scFv) comprises an anti-WTMC antibody moiety, such as any of the anti-WTMC antibody moieties described in the “Anti-WTMC Antibodies” Section.

### Chimeric Receptors and Effector Cells

In some aspects, the anti-WTMC construct provided herein in is a chimeric receptor comprising an anti-WTMC antibody moiety (such as any one of the anti-WTMC antibody moieties described in the “Anti-WTMC Antibody Moieties” section). As described in further detail elsewhere herein, the present disclosure also provides chimeric receptor effector cells (e.g., T cells) that comprise, express, or as associated with an anti-WTMC chimeric receptor. Such effector cells are also referred to herein as an “anti-WTMC effector cells”. Anti-WTMC chimeric receptors and/or effector cells within the scope of the present application include, without limitation, e.g., anti-WTMC CARs, anti-WTMC chimeric antibody-T cell receptor constructs (caTCRs), anti-WTMC chimeric signaling receptors (CSRs), and others, as described herein below.

### Chimeric Antigen Receptors (CARs)

The present application in some embodiments provides chimeric antigen receptors (CARs). In some embodiments, the present disclosure also provides CAR effector cells (e.g., T cells) that comprise, express, or as associated with a CAR. Such effector cells are also referred to herein as “CAR effector cells”, e.g., “CAR immune cells” or “CAR T cells”). In some embodiments, the anti-WTMC construct provided herein in is a CAR (also referred to herein as an “anti-WTMC CAR”) comprising an anti-WTMC antibody moiety (such as any one of the anti-WTMC antibody moieties described in the “Anti-WTMC Antibody Moieties” section). In some embodiments, the present disclosure also provides CAR effector cells (e.g., T cells) that comprise, express, or as associated with an anti-WTMC CAR. Such effector cells are also referred to herein as “anti-WTMC CAR effector cells”, e.g., “anti-WTMC CAR immune cells” or “anti-WTMC CAR T cells”). The features of CARs described in this section apply to both anti-WTMC CARs and CARs that do not target WTMC.

In some embodiments, the chimeric receptor is a chimeric antigen receptor (CAR), such as an anti-WTMC CAR. In some embodiments, the CAR is a bispecific CAR. In some embodiments, the CAR comprises a) an extracellular domain comprising an antibody moiety that specifically binds to a target and b) an intracellular signaling domain. In some embodiments, the CAR comprises a transmembrane domain between the extracellular domain and the intracellular domain. In some embodiments, the CAR further comprises a spacer. In some embodiments, the spacer connects the extracellular domain and the transmembrane domain of the CAR. In some embodiments, the spacer connects the intracellular domain and the transmembrane domain of the CAR. In some embodiments, the spacer domain is any oligo- or polypeptide that functions to link the transmembrane domain to the extracellular domain or the intracellular domain in the polypeptide chain. For example, a spacer domain may comprise up to about 300 amino acids, including for example between about 10 and about 100 amino acids, or between about 25 and about 50 amino acids.

In some embodiments, the CAR comprises transmembrane domain that naturally is associated with one of the sequences in the CAR's intracellular domain of. For example, if a CAR intracellular domain comprises a CD28 co-stimulatory sequence, the transmembrane domain of the CAR is derived from the CD28 transmembrane domain. In some embodiments, the CAR comprises a transmembrane domain that has been selected or modified by amino acid substitution to minimize interactions with other members of the receptor complex and/or to avoid binding to the transmembrane domains of the same or different surface membrane proteins.

The intracellular signaling domain of the CAR is responsible for activation of at least one of the normal effector functions of the immune cell in which the CAR is expressed. Effector function of a T cell, for example, may be cytolytic activity or helper activity, including the secretion of cytokines. Thus, in some embodiments, the term “intracellular signaling domain” refers to the portion of a CAR that transduces the effector function signal and directs the cell to perform a specialized function.

It is known that signals generated through the TCR alone are insufficient for full activation of the T cell and that a secondary or co-stimulatory signal is also required. Thus, T cell activation can be said to be mediated by two distinct classes of intracellular signaling sequences: those that initiate antigen-dependent primary activation through the TCR (primary signaling sequences or primary immune cell signaling sequences) and those that act in an antigen-independent manner to provide a secondary or co-stimulatory signal (co-stimulatory signaling sequences).

Primary signaling sequences, or primary immune cell signaling sequences, regulate primary activation of the TCR complex in a stimulatory way or in an inhibitory way. Primary signaling sequences that act in a stimulatory manner may contain signaling motifs which are known as immunoreceptor tyrosine-based activation motifs (or ITAMs). Thus, in some embodiments, the CAR comprises one or more ITAMs. In some embodiments, the CAR comprises a primary immune cell signaling sequence derived from, without limitation, TCRζ, FcRγ, FcRβ, CD3γ, CD3δ, CD3ε, CD5, CD22, CD79a, CD79b, and CD66d. In some embodiments, the CAR further comprises a costimulatory signaling sequence. In some embodiments, the costimulatory signaling sequence is a portion of the CD intracellular domain of a costimulatory molecule including, for example, CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, and the like. In some embodiments, the CAR comprises more than one costimulatory signaling sequence.

In some embodiments, the CAR comprises a primary immune cell signaling sequence derived from CD3ζ. In some embodiments, the CAR comprises a primary immune cell signaling sequence derived from CD3ζ by itself or combined with any other desired intracellular signaling sequence(s) useful in the context of the CAR provided herein. For example, in some embodiments, the CAR comprises an intracellular domain that comprises a primary immune cell signaling sequence derived from CD3ζ and a costimulatory signaling sequence. In some embodiments, the costimulatory signaling sequence is a portion of the intracellular domain of a costimulatory molecule including, for example, CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, and the like. In some embodiments, the costimulatory signaling sequence is derived from, e.g., CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, and the like. In some embodiments, the CAR comprises more than one costimulatory signaling sequence.

In some embodiments, the CAR comprises the intracellular signaling domain that comprises a primary immune cell signaling sequence derived from CD3ζ and a costimulatory signaling sequence derived from CD28. In some embodiments, the CAR comprises the intracellular signaling domain that comprises a primary immune cell signaling sequence derived from CD3ζ and a costimulatory signaling sequence derived from CD30. In some embodiments, the CAR comprises the intracellular signaling domain that comprises a primary immune cell signaling sequence derived from CD3ζ and a costimulatory signaling sequence derived from 4-1BB. In some embodiments, the intracellular signaling domain of the CAR comprises a primary immune cell signaling sequence derived from CD3ζ and costimulatory signaling sequences derived from CD28 and 4-1BB. In some embodiments, the intracellular signaling domain of the CAR comprises a primary immune cell signaling sequence derived from CD3ζ and costimulatory signaling sequences derived from CD28 and OX40 or CD28 and ICOS. In some embodiments, the intracellular signaling domain of the CAR comprises a primary immune cell signaling sequence derived from CD3ζ and costimulatory signaling sequences derived from at least one of CD27, CD28, 4-1BB (CD137), OX40, CD30, CD40, PD-1, ICOS, LFA-1, CD2, CD7, LIGHT, NKG2C, B7-H3, a ligand that specifically binds with CD83.

In some embodiments, the CAR comprises a target-binding domain (e.g., an antibody moiety) that specifically binds a WTMC. In some embodiments, the CAR comprises a target-binding domain (e.g., an antibody moiety) that does not specifically bind a WTMC. In some embodiments, the CAR comprises two or more target-binding domains (e.g., antibody moieties) that specifically bind to the same or different targets. In some embodiments, the CAR is monospecific. In some embodiments, the CAR is multispecific, such as bispecific.

In some embodiments, the target-binding domain is a single-chain antibody moiety, such as a single chain Fv (scFv). In some embodiments, the target-binding domain specifically binds a cell surface protein. In some embodiments, the target-binding domain specifically binds a complex comprising a peptide and an MHC molecule.

In some embodiments, the target-binding domain specifically binds a target expressed or present on a cancer cell. In some embodiments, the target-binding domain specifically binds a target expressed or present on a WT1-positive cell. In some embodiments, the target-binding domain specifically binds CD33, CD371, CD123, and/or CD15. In some embodiments, the target-binding domain specifically binds a peptide/MHC complex, wherein the peptide is WT1. In some embodiments, the target-binding domain specifically binds a target expressed or present on a cancer type.

In some embodiments, there is provided an anti-WTMC CAR comprising a) an extracellular domain comprising any one of the anti-WTMC antibody moieties described herein (e.g., scFv) that specifically binds to a complex comprising a WT1 peptide (e.g., WT1-RMF) and an MHC class I protein, b) a transmembrane domain, and c) an intracellular signaling domain. In some embodiments, the WT1 peptide (e.g., WT1-RMF) comprises SEQ ID NO: 113. In some embodiments, the antibody moiety specifically recognizes amino acid residues FPNA (SEQ ID NO: 349) of SEQ ID NO: 113. In some embodiments, the MHC class I protein is HLA-A02. In some embodiments, the MHC class I protein is HLA-A*02:01. In some embodiments, the intracellular signaling domain is capable of activating an immune cell. In some embodiments, the intracellular signaling domain comprises a primary signaling sequence and a co-stimulatory signaling sequence. In some embodiments, the primary signaling sequence comprises a CD3ζ intracellular signaling sequence. In some embodiments, the co-stimulatory signaling sequence comprises a CD28 and/or 4-1BB intracellular signaling sequence. In some embodiments, the intracellular domain comprises a CD3ζ intracellular signaling sequence and a CD28 and/or 4-1BB intracellular signaling sequence. In some embodiments, the anti-WTMC antibody moiety cross-reacts with at least one (such as at least any of 2, 3, 4, or 5) complex comprising the WT1 peptide (e.g., WT1-RMF) and a different subtype of the MHC class I protein.

In some embodiments, the anti-WTMC CAR comprises: a) any one of the anti-WTMC antibody moieties described herein; b) a transmembrane module; and c) an intracellular signaling domain. In some embodiments, the anti-WTMC CAR comprises: (a) an anti-WTMC antibody moiety comprising a HC-CDR1 comprising an amino acid sequence set forth in SEQ ID NO: 1; a HC-CDR2 comprising an amino acid sequence set forth in SEQ ID NO: 2; a HC-CDR3 comprising an amino acid sequence set forth in SEQ ID NO: 3; a LC-CDR1 comprising an amino acid sequence set forth in SEQ ID NO: 4; a LC-CDR2 comprising an amino acid sequence set forth in SEQ ID NO: 5; and a LC-CDR3 comprising an amino acid sequence set forth in SEQ ID NO: 6; (b) a transmembrane domain; and (c) an intracellular signaling domain. In some embodiments, the anti-WTMC CAR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 85 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 86. In some embodiments, the anti-WTMC CAR comprises an anti-WTMC antibody moiety comprising a V_{H} domain comprising SEQ ID NO: 85 and a V_{L} domain comprising SEQ ID NO: 86.

In some embodiments, the anti-WTMC CAR comprises: a) any one of the anti-WTMC antibody moieties described herein; b) a transmembrane module; and c) an intracellular signaling domain. In some embodiments, the anti-WTMC CAR comprises: (a) an anti-WTMC antibody moiety comprising a HC-CDR1 comprising an amino acid sequence set forth in SEQ ID NO: 7; a HC-CDR2 comprising an amino acid sequence set forth in SEQ ID NO: 8; a HC-CDR3 comprising an amino acid sequence set forth in SEQ ID NO: 9; a LC-CDR1 comprising an amino acid sequence set forth in SEQ ID NO: 10; a LC-CDR2 comprising an amino acid sequence set forth in SEQ ID NO: 11; and a LC-CDR3 comprising an amino acid sequence set forth in SEQ ID NO: 12; (b) a transmembrane domain; and (c) an intracellular signaling domain. In some embodiments, the anti-WTMC CAR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 87 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 88. In some embodiments, the anti-WTMC CAR comprises an anti-WTMC antibody moiety comprising a V_{H} domain comprising SEQ ID NO: 87 and a V_{L} domain comprising SEQ ID NO: 88.

In some embodiments, the anti-WTMC CAR comprises: a) any one of the anti-WTMC antibody moieties described herein; b) a transmembrane module; and c) an intracellular signaling domain. In some embodiments, the anti-WTMC CAR comprises: (a) an anti-WTMC antibody moiety comprising a HC-CDR1 comprising an amino acid sequence set forth in SEQ ID NO: 13; a HC-CDR2 comprising an amino acid sequence set forth in SEQ ID NO: 14; a HC-CDR3 comprising an amino acid sequence set forth in SEQ ID NO: 15; a LC-CDR1 comprising an amino acid sequence set forth in SEQ ID NO: 16; a LC-CDR2 comprising an amino acid sequence set forth in SEQ ID NO: 17; and a LC-CDR3 comprising an amino acid sequence set forth in SEQ ID NO: 18; (b) a transmembrane domain; and (c) an intracellular signaling domain. In some embodiments, the anti-WTMC CAR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 89 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 90. In some embodiments, the anti-WTMC CAR comprises an anti-WTMC antibody moiety comprising a V_{H} domain comprising SEQ ID NO: 89 and a V_{L} domain comprising SEQ ID NO: 90.

In some embodiments, the anti-WTMC CAR comprises: a) any one of the anti-WTMC antibody moieties described herein; b) a transmembrane module; and c) an intracellular signaling domain. In some embodiments, the anti-WTMC CAR comprises: (a) an anti-WTMC antibody moiety comprising a HC-CDR1 comprising an amino acid sequence set forth in SEQ ID NO: 19; a HC-CDR2 comprising an amino acid sequence set forth in SEQ ID NO: 20; a HC-CDR3 comprising an amino acid sequence set forth in SEQ ID NO: 21; a LC-CDR1 comprising an amino acid sequence set forth in SEQ ID NO: 22; a LC-CDR2 comprising an amino acid sequence set forth in SEQ ID NO: 23; and a LC-CDR3 comprising an amino acid sequence set forth in SEQ ID NO: 24; (b) a transmembrane domain; and (c) an intracellular signaling domain. In some embodiments, the anti-WTMC CAR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 91 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 92. In some embodiments, the anti-WTMC CAR comprises an anti-WTMC antibody moiety comprising a V_{H} domain comprising SEQ ID NO: 91 and a V_{L} domain comprising SEQ ID NO: 92.

In some embodiments, the anti-WTMC CAR comprises: a) any one of the anti-WTMC antibody moieties described herein; b) a transmembrane module; and c) an intracellular signaling domain. In some embodiments, the anti-WTMC CAR comprises: (a) an anti-WTMC antibody moiety comprising a HC-CDR1 comprising an amino acid sequence set forth in SEQ ID NO: 25; a HC-CDR2 comprising an amino acid sequence set forth in SEQ ID NO: 26; a HC-CDR3 comprising an amino acid sequence set forth in SEQ ID NO: 27; a LC-CDR1 comprising an amino acid sequence set forth in SEQ ID NO: 28; a LC-CDR2 comprising an amino acid sequence set forth in SEQ ID NO: 29; and a LC-CDR3 comprising an amino acid sequence set forth in SEQ ID NO: 30; (b) a transmembrane domain; and (c) an intracellular signaling domain. In some embodiments, the anti-WTMC CAR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 93 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 94. In some embodiments, the anti-WTMC CAR comprises an anti-WTMC antibody moiety comprising a V_{H} domain comprising SEQ ID NO: 93 and a V_{L} domain comprising SEQ ID NO: 94.

In some embodiments, the anti-WTMC CAR comprises: a) any one of the anti-WTMC antibody moieties described herein; b) a transmembrane module; and c) an intracellular signaling domain. In some embodiments, the anti-WTMC CAR comprises: (a) an anti-WTMC antibody moiety comprising a HC-CDR1 comprising an amino acid sequence set forth in SEQ ID NO: 31; a HC-CDR2 comprising an amino acid sequence set forth in SEQ ID NO: 32; a HC-CDR3 comprising an amino acid sequence set forth in SEQ ID NO: 33; a LC-CDR1 comprising an amino acid sequence set forth in SEQ ID NO: 34; a LC-CDR2 comprising an amino acid sequence set forth in SEQ ID NO: 35; and a LC-CDR3 comprising an amino acid sequence set forth in SEQ ID NO: 36; (b) a transmembrane domain; and (c) an intracellular signaling domain. In some embodiments, the anti-WTMC CAR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 95 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 96. In some embodiments, the anti-WTMC CAR comprises an anti-WTMC antibody moiety comprising a V_{H} domain comprising SEQ ID NO: 95 and a V_{L} domain comprising SEQ ID NO: 96.

In some embodiments, the anti-WTMC CAR comprises: a) any one of the anti-WTMC antibody moieties described herein; b) a transmembrane module; and c) an intracellular signaling domain. In some embodiments, the anti-WTMC CAR comprises: (a) an anti-WTMC antibody moiety comprising a HC-CDR1 comprising an amino acid sequence set forth in SEQ ID NO: 37; a HC-CDR2 comprising an amino acid sequence set forth in SEQ ID NO: 38; a HC-CDR3 comprising an amino acid sequence set forth in SEQ ID NO: 39; a LC-CDR1 comprising an amino acid sequence set forth in SEQ ID NO: 40; a LC-CDR2 comprising an amino acid sequence set forth in SEQ ID NO: 41; and a LC-CDR3 comprising an amino acid sequence set forth in SEQ ID NO: 42; (b) a transmembrane domain; and (c) an intracellular signaling domain. In some embodiments, the anti-WTMC CAR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 97 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 98. In some embodiments, the anti-WTMC CAR comprises an anti-WTMC antibody moiety comprising a V_{H} domain comprising SEQ ID NO: 97 and a V_{L} domain comprising SEQ ID NO: 98.

In some embodiments, the anti-WTMC CAR comprises: a) any one of the anti-WTMC antibody moieties described herein; b) a transmembrane module; and c) an intracellular signaling domain. In some embodiments, the anti-WTMC CAR comprises: (a) an anti-WTMC antibody moiety comprising a HC-CDR1 comprising an amino acid sequence set forth in SEQ ID NO: 43; a HC-CDR2 comprising an amino acid sequence set forth in SEQ ID NO: 44; a HC-CDR3 comprising an amino acid sequence set forth in SEQ ID NO: 45; a LC-CDR1 comprising an amino acid sequence set forth in SEQ ID NO: 46; a LC-CDR2 comprising an amino acid sequence set forth in SEQ ID NO: 47; and a LC-CDR3 comprising an amino acid sequence set forth in SEQ ID NO: 48; (b) a transmembrane domain; and (c) an intracellular signaling domain. In some embodiments, the anti-WTMC CAR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 99 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 100. In some embodiments, the anti-WTMC CAR comprises an anti-WTMC antibody moiety comprising a V_{H} domain comprising SEQ ID NO: 99 and a V_{L} domain comprising SEQ ID NO: 100.

In some embodiments, the anti-WTMC CAR comprises: a) any one of the anti-WTMC antibody moieties described herein; b) a transmembrane module; and c) an intracellular signaling domain. In some embodiments, the anti-WTMC CAR comprises: (a) an anti-WTMC antibody moiety comprising a HC-CDR1 comprising an amino acid sequence set forth in SEQ ID NO: 49; a HC-CDR2 comprising an amino acid sequence set forth in SEQ ID NO: 50; a HC-CDR3 comprising an amino acid sequence set forth in SEQ ID NO: 51; a LC-CDR1 comprising an amino acid sequence set forth in SEQ ID NO: 52; a LC-CDR2 comprising an amino acid sequence set forth in SEQ ID NO: 53; and a LC-CDR3 comprising an amino acid sequence set forth in SEQ ID NO: 54; (b) a transmembrane domain; and (c) an intracellular signaling domain. In some embodiments, the anti-WTMC CAR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 101 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 102. In some embodiments, the anti-WTMC CAR comprises an anti-WTMC antibody moiety comprising a V_{H} domain comprising SEQ ID NO: 101 and a V_{L} domain comprising SEQ ID NO: 102.

In some embodiments, the anti-WTMC CAR comprises: a) any one of the anti-WTMC antibody moieties described herein; b) a transmembrane module; and c) an intracellular signaling domain. In some embodiments, the anti-WTMC CAR comprises: (a) an anti-WTMC antibody moiety comprising a HC-CDR1 comprising an amino acid sequence set forth in SEQ ID NO: 55; a HC-CDR2 comprising an amino acid sequence set forth in SEQ ID NO: 56; a HC-CDR3 comprising an amino acid sequence set forth in SEQ ID NO: 57; a LC-CDR1 comprising an amino acid sequence set forth in SEQ ID NO: 58; a LC-CDR2 comprising an amino acid sequence set forth in SEQ ID NO: 59; and a LC-CDR3 comprising an amino acid sequence set forth in SEQ ID NO: 60; (b) a transmembrane domain; and (c) an intracellular signaling domain. In some embodiments, the anti-WTMC CAR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 103 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 104. In some embodiments, the anti-WTMC CAR comprises an anti-WTMC antibody moiety comprising a V_{H} domain comprising SEQ ID NO: 103 and a V_{L} domain comprising SEQ ID NO: 104.

In some embodiments, the anti-WTMC CAR comprises: a) any one of the anti-WTMC antibody moieties described herein; b) a transmembrane module; and c) an intracellular signaling domain. In some embodiments, the anti-WTMC CAR comprises: (a) an anti-WTMC antibody moiety comprising a HC-CDR1 comprising an amino acid sequence set forth in SEQ ID NO: 61; a HC-CDR2 comprising an amino acid sequence set forth in SEQ ID NO: 62; a HC-CDR3 comprising an amino acid sequence set forth in SEQ ID NO: 63; a LC-CDR1 comprising an amino acid sequence set forth in SEQ ID NO: 64; a LC-CDR2 comprising an amino acid sequence set forth in SEQ ID NO: 65; and a LC-CDR3 comprising an amino acid sequence set forth in SEQ ID NO: 66; (b) a transmembrane domain; and (c) an intracellular signaling domain. In some embodiments, the anti-WTMC CAR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 105 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 106. In some embodiments, the anti-WTMC CAR comprises an anti-WTMC antibody moiety comprising a V_{H} domain comprising SEQ ID NO: 105 and a V_{L} domain comprising SEQ ID NO: 106.

In some embodiments, the anti-WTMC CAR comprises: a) any one of the anti-WTMC antibody moieties described herein; b) a transmembrane module; and c) an intracellular signaling domain. In some embodiments, the anti-WTMC CAR comprises: (a) an anti-WTMC antibody moiety comprising a HC-CDR1 comprising an amino acid sequence set forth in SEQ ID NO: 67; a HC-CDR2 comprising an amino acid sequence set forth in SEQ ID NO: 68; a HC-CDR3 comprising an amino acid sequence set forth in SEQ ID NO: 69; a LC-CDR1 comprising an amino acid sequence set forth in SEQ ID NO: 70; a LC-CDR2 comprising an amino acid sequence set forth in SEQ ID NO: 71; and a LC-CDR3 comprising an amino acid sequence set forth in SEQ ID NO: 72; (b) a transmembrane domain; and (c) an intracellular signaling domain. In some embodiments, the anti-WTMC CAR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 107 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 108. In some embodiments, the anti-WTMC CAR comprises an anti-WTMC antibody moiety comprising a V_{H} domain comprising SEQ ID NO: 107 and a V_{L} domain comprising SEQ ID NO: 108.

In some embodiments, the anti-WTMC CAR comprises: a) any one of the anti-WTMC antibody moieties described herein; b) a transmembrane module; and c) an intracellular signaling domain. In some embodiments, the anti-WTMC CAR comprises: (a) an anti-WTMC antibody moiety comprising a HC-CDR1 comprising an amino acid sequence set forth in SEQ ID NO: 73; a HC-CDR2 comprising an amino acid sequence set forth in SEQ ID NO: 74; a HC-CDR3 comprising an amino acid sequence set forth in SEQ ID NO: 75; a LC-CDR1 comprising an amino acid sequence set forth in SEQ ID NO: 76; a LC-CDR2 comprising an amino acid sequence set forth in SEQ ID NO: 77; and a LC-CDR3 comprising an amino acid sequence set forth in SEQ ID NO: 78; (b) a transmembrane domain; and (c) an intracellular signaling domain. In some embodiments, the anti-WTMC CAR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 109 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 110. In some embodiments, the anti-WTMC CAR comprises an anti-WTMC antibody moiety comprising a V_{H} domain comprising SEQ ID NO: 109 and a V_{L} domain comprising SEQ ID NO: 110.

In some embodiments, the anti-WTMC CAR comprises: a) any one of the anti-WTMC antibody moieties described herein; b) a transmembrane module; and c) an intracellular signaling domain. In some embodiments, the anti-WTMC CAR comprises: (a) an anti-WTMC antibody moiety comprising a HC-CDR1 comprising an amino acid sequence set forth in SEQ ID NO: 79; a HC-CDR2 comprising an amino acid sequence set forth in SEQ ID NO: 80; a HC-CDR3 comprising an amino acid sequence set forth in SEQ ID NO: 81; a LC-CDR1 comprising an amino acid sequence set forth in SEQ ID NO: 82; a LC-CDR2 comprising an amino acid sequence set forth in SEQ ID NO: 83; and a LC-CDR3 comprising an amino acid sequence set forth in SEQ ID NO: 84; (b) a transmembrane domain; and (c) an intracellular signaling domain. In some embodiments, the anti-WTMC CAR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 111 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 112. In some embodiments, the anti-WTMC CAR comprises an anti-WTMC antibody moiety comprising a V_{H} domain comprising SEQ ID NO: 111 and a V_{L} domain comprising SEQ ID NO: 112.

In any and all embodiments of the anti-WTMC CAR disclosed herein, the intracellular signaling domain is capable of activating an immune cell. In some embodiments, the intracellular signaling domain comprises a primary signaling sequence and a co-stimulatory signaling sequence. In some embodiments, the primary signaling sequence comprises a CD3ζ intracellular signaling sequence. In some embodiments, the co-stimulatory signaling sequence comprises a CD28 and/or 4-1BB intracellular signaling sequence. In some embodiments, the intracellular domain comprises a CD3ζ intracellular signaling sequence and a CD28 and/or 4-1BB intracellular signaling sequence. In some embodiments, the co-stimulatory signaling sequence comprises a CD28 and/or OX40 intracellular signaling sequence. In some embodiments, the intracellular domain comprises a CD3ζ intracellular signaling sequence and a CD28 and/or ICOS intracellular signaling sequence. In certain embodiments, the intracellular signaling domain comprises a CD3ζ intracellular signaling sequence and one or more costimulatory signaling sequences derived from CD27, CD28, 4-1BB (CD137), OX40, CD30, CD40, PD-1, ICOS, LFA-1, CD2, CD7, LIGHT, NKG2C, B7-H3, a ligand that specifically binds with CD83.

In some embodiments, the anti-WTMC CAR comprises an anti-WTMC antibody moiety comprising heavy chain and light chain variable domains comprising the amino acid sequence of SEQ ID NOs: 85 and 86, respectively, SEQ ID NOs: 87 and 88, respectively, SEQ ID NOs: 89 and 90, respectively, SEQ ID NOs: 91 and 92, respectively, in SEQ ID NOs: 93 and 94, respectively, SEQ ID NOs: 95 and 96, respectively, SEQ ID NOs: 97 and 98, respectively, SEQ ID NOs: 99 and 100, respectively, SEQ ID NOs: 101 and 102, respectively, in SEQ ID NOs: 103 and 104, respectively, SEQ ID NOs: 105 and 106, respectively, SEQ ID NOs: 107 and 108, respectively, SEQ ID NOs: 109 and 110, respectively, SEQ ID NOs: 111 and 112, respectively, SEQ ID NOs: 123 and 102, respectively, SEQ ID NOs: 124 and 102, respectively, SEQ ID NOs: 125 and 102, respectively, SEQ ID NOs: 123 and 126, respectively, SEQ ID NOs: 124 and 126, respectively, SEQ ID NOs: 125 and 126, respectively, SEQ ID NOs: 123 and 127, respectively, SEQ ID NOs: 124 and 127, respectively, SEQ ID NOs: 125 and 127, respectively, SEQ ID NOs: 123 and 128, respectively, SEQ ID NOs: 124 and 128, respectively, SEQ ID NOs: 125 and 128, respectively, SEQ ID NOs: 123 and 129, respectively, SEQ ID NOs: 124 and 129, respectively, SEQ ID NOs: 125 and 129, respectively, SEQ ID NOs: 101 and 126, respectively, SEQ ID NOs: 101 and 127, respectively, SEQ ID NOs: 101 and 128, respectively, SEQ ID NOs: 101 and 129, respectively, SEQ ID NOs: 130 and 110, respectively, SEQ ID NOs: 130 and 134, respectively, SEQ ID NOs: 130 and 135, respectively, SEQ ID NOs: 130 and 136, respectively, SEQ ID NOs: 131 and 110, respectively, SEQ ID NOs: 131 and 134, respectively, SEQ ID NOs: 131 and 135, respectively, SEQ ID NOs: 131 and 136, respectively, SEQ ID NOs: 132 and 110, respectively, SEQ ID NOs: 132 and 134, respectively, SEQ ID NOs: 132 and 135, respectively, SEQ ID NOs: 132 and 136, respectively, SEQ ID NOs: 133 and 110, respectively, SEQ ID NOs: 133 and 134, respectively, SEQ ID NOs: 133 and 135, respectively, SEQ ID NOs: 133 and 136, respectively, SEQ ID NOs: 109 and 134, respectively, SEQ ID NOs: 109 and 135, respectively, SEQ ID NOs: 109 and 136, respectively; or variants thereof having, individually, at least about 95% (for example at least about any of 96%, 97%, 98%, or 99%) sequence identity.

In some embodiments, the anti-WTMC CAR comprises an anti-WTMC antibody moiety comprising heavy chain and light chain variable domains comprising the amino acid sequence of SEQ ID NOs: 85 and 86, respectively, SEQ ID NOs: 87 and 88, respectively, SEQ ID NOs: 89 and 90, respectively, SEQ ID NOs: 91 and 92, respectively, in SEQ ID NOs: 93 and 94, respectively, SEQ ID NOs: 95 and 96, respectively, SEQ ID NOs: 97 and 98, respectively, SEQ ID NOs: 99 and 100, respectively, SEQ ID NOs: 101 and 102, respectively, in SEQ ID NOs: 103 and 104, respectively, SEQ ID NOs: 105 and 106, respectively, SEQ ID NOs: 107 and 108, respectively, SEQ ID NOs: 109 and 110, respectively, SEQ ID NOs: 111 and 112, respectively, SEQ ID NOs: 123 and 102, respectively, SEQ ID NOs: 124 and 102, respectively, SEQ ID NOs: 125 and 102, respectively, SEQ ID NOs: 123 and 126, respectively, SEQ ID NOs: 124 and 126, respectively, SEQ ID NOs: 125 and 126, respectively, SEQ ID NOs: 123 and 127, respectively, SEQ ID NOs: 124 and 127, respectively, SEQ ID NOs: 125 and 127, respectively, SEQ ID NOs: 123 and 128, respectively, SEQ ID NOs: 124 and 128, respectively, SEQ ID NOs: 125 and 128, respectively, SEQ ID NOs: 123 and 129, respectively, SEQ ID NOs: 124 and 129, respectively, SEQ ID NOs: 125 and 129, respectively, SEQ ID NOs: 101 and 126, respectively, SEQ ID NOs: 101 and 127, respectively, SEQ ID NOs: 101 and 128, respectively, SEQ ID NOs: 101 and 129, respectively, SEQ ID NOs: 130 and 110, respectively, SEQ ID NOs: 130 and 134, respectively, SEQ ID NOs: 130 and 135, respectively, SEQ ID NOs: 130 and 136, respectively, SEQ ID NOs: 131 and 110, respectively, SEQ ID NOs: 131 and 134, respectively, SEQ ID NOs: 131 and 135, respectively, SEQ ID NOs: 131 and 136, respectively, SEQ ID NOs: 132 and 110, respectively, SEQ ID NOs: 132 and 134, respectively, SEQ ID NOs: 132 and 135, respectively, SEQ ID NOs: 132 and 136, respectively, SEQ ID NOs: 133 and 110, respectively, SEQ ID NOs: 133 and 134, respectively, SEQ ID NOs: 133 and 135, respectively, SEQ ID NOs: 133 and 136, respectively, SEQ ID NOs: 109 and 134, respectively, SEQ ID NOs: 109 and 135, respectively, or SEQ ID NOs: 109 and 136, respectively.

In some embodiments, the anti-WTMC CAR comprises: a) the anti-WTMC antibody moiety of Clone 1, 2, 10, 11, 12, 14, 15, 17, 18, 26, 30, 32, 34, or 36; b) a transmembrane module; and c) an intracellular signaling domain. In some embodiments, the intracellular signaling domain comprises a CD3ζ intracellular signaling sequence and a CD28 intracellular signaling sequence. In some embodiments, the intracellular signaling domain comprises the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 289. In some embodiments, the intracellular signaling domain comprises the amino acid sequence of SEQ ID NO: 289. In some embodiments, the intracellular signaling domain comprises the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 283. In some embodiments, the intracellular signaling domain comprises the amino acid sequence of SEQ ID NO: 283. In some embodiments, the intracellular signaling domain comprises the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 289 and 283. In some embodiments, the intracellular signaling domain comprises the amino acid sequence of SEQ ID NO: 289 and 283.

In some embodiments, the anti-WTMC CAR comprises: a) the anti-WTMC antibody moiety of Clone 1, 2, 10, 11, 12, 14, 15, 17, 18, 26, 30, 32, 34, or 36; b) a transmembrane module; and c) an intracellular signaling domain. In some embodiments, the intracellular domain comprises a CD3ζ intracellular signaling sequence and a CD28 intracellular signaling sequence and transmembrane domain. In some embodiments, the anti-WTMC CAR comprises the anti-WTMC antibody moiety of Clone 18 or Clone 34. In some embodiments, the intracellular signaling domain comprises the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 289. In some embodiments, the intracellular signaling domain comprises the amino acid sequence of SEQ ID NO: 289. In some embodiments, the intracellular signaling domain comprises the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 290. In some embodiments, the intracellular signaling domain comprises the amino acid sequence of SEQ ID NO: 290.

In some embodiments, the intracellular signaling domain comprises the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 290. In some embodiments, the intracellular signaling domain comprises the amino acid sequence of SEQ ID NO: 290.

In some embodiments, the intracellular signaling domain comprises a CD3ζ intracellular signaling sequence and a costimulatory intracellular signaling sequence (e.g., CD27, CD28, 4-1BB (CD137), OX40, CD30, CD40, PD-1, ICOS, LFA-1, CD2, CD7, LIGHT, NKG2C, B7-H3, a ligand that specifically binds with CD83) and transmembrane domain. In some embodiments, the intracellular signaling domain comprises the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 289. In some embodiments, the intracellular signaling domain comprises the amino acid sequence of SEQ ID NO: 289. In some embodiments, the intracellular signaling domain comprises the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 284. In some embodiments, the intracellular signaling domain comprises the amino acid sequence of SEQ ID NO: 284.

In some embodiments, the intracellular signaling domain comprises the amino acid sequence of SEQ ID NO: 297. In some embodiments, the intracellular signaling domain comprises the amino acid sequence of SEQ ID NO: 289 and 284. In some embodiments, the intracellular signaling domain comprises the amino acid sequence of SEQ ID NO: 289 and 297.

Chimeric Antibody-T Cell Receptor Constructs (caTCRs)

In some embodiments, provided herein is a chimeric antibody-T cell receptor construct (caTCR). Such construct is also referred to herein as a “caTCR.” Exemplary caTCRs are discussed in U.S. Ser. No. 10/098,951B2, the contents of which are incorporated herein by reference in their entirety. In some embodiments, the caTCR specifically bind to a target and is capable of associating with at least one TCR-associated signaling molecule (such as CD3δε, CD3γε, and/or CD3ζζ). In some embodiments, the caTCR does not comprise a variable domain of a TCR. In some embodiments, the caTCR does not comprise a constant domain of a TCR. In some embodiments, the caTCR is a bispecific caTCR. The features of caTCRs described in this section apply to both anti-WTMC caTCR and caTCRs that do not target WTMC.

As described in further detail below, the present disclosure also provides an effector cell (e.g., T cell) that comprises, expresses, or is associated with a caTCR, such as an anti-WTMC caTCR. Such effector cells are also referred to herein as a “caTCR effector cells” (e.g., “caTCR T cells”), including “anti-WTMC caTCR effector cells” (e.g., “anti-WTMC caTCR T cells”).

In some embodiments, the caTCR comprises a) an antigen-binding module, and b) a T cell receptor module (TCRM) comprising a first TCR domain (TCRD) comprising a first TCR transmembrane domain (TCR-TM) derived from one of the transmembrane domains of a naturally occurring TCR (such as an αβTCR or a γδTCR) and a second TCRD comprising a second TCR-TM derived from the other transmembrane domain of the naturally occurring TCR (such as an αβTCR or a γδTCR), wherein the TCRM is capable of associating with at least one TCR-associated signaling molecule (such as CD3δε, CD3γε, and/or CD3ζζ), and wherein the antibody moiety is linked to the first and/or second TCRDs. In some embodiments, the first TCR-TM and the second TCR-TM are derived from a γ/δ TCR. In some embodiments, the first TCR-TM is derived from a TCR γ chain and the second TCR-TM is derived from a TCR δ chain. In some embodiments, the first TCR-TM is derived from a TCR δ chain and the second TCR-TM is derived from a TCR γ chain. In some embodiments, the first TCR-TM and the second TCR-TM are derived from an α/β TCR. In some embodiments, the first TCR-TM is derived from a TCR α chain and the second TCR-TM is derived from a TCR β chain. In some embodiments, the first TCR-TM is derived from a TCR β chain and the second TCR-TM is derived from a TCR α chain. In some embodiments, the caTCR comprises naturally occurring TCR domains. In some embodiments, the caTCR comprises at least one non-naturally occurring TCR domain. For example, the γ/δ TCR, the α/β TCR, the TCR γ chain, the TCR δ chain, the TCR α chain, and/or the TCR β chain may ne naturally occurring or non-naturally occurring. The antigen-binding module of the anti-WTMC caTCR provides the antigen specificity and a TCRM that allows for CD3 recruitment and signaling. In some embodiments, the antigen-binding module is not a naturally occurring T cell receptor antigen-binding moiety. In some embodiments, the antigen-binding module is linked to the N-terminus of a polypeptide chain in the TCRM. In some embodiments, the antigen binding module is an antibody moiety selected from the group consisting of. a Fab, a Fab′, a F(ab′)₂, an Fv, or an scFv. The TCRM comprises a transmembrane module derived from the transmembrane domains of one or more TCRs (TCR-TMs), such as an αβ and/or γδ TCR, and optionally further comprises one or both of the connecting peptides or fragments thereof of a TCR and/or one or more TCR intracellular domains or fragments thereof. In some embodiments, the TCRM comprises two polypeptide chains, each polypeptide chain comprising, from N-terminus to C-terminus, a connecting peptide, a transmembrane domain, and optionally a TCR intracellular domain. In some embodiments, the TCRM comprises one or more non-naturally occurring TCR domains. For example, in some embodiments, the TCRM comprises one or two non-naturally occurring TCR transmembrane domains. A non-naturally occurring TCR domain may be a corresponding domain of a naturally occurring TCR modified by substitution of one or more amino acids, and/or by replacement of a portion of the corresponding domain with a portion of an analogous domain from another TCR. In some embodiments, the anti-WTMC caTCR comprises a first polypeptide chain and a second polypeptide chain, wherein the first and second polypeptide chains together form the antigen-binding module and the TCRM. In some embodiments, the first and second polypeptide chains are separate polypeptide chains, and the caTCR is a multimer, such as a dimer. In some embodiments, the first and second polypeptide chains are covalently linked, such as by a peptide linkage, or by another chemical linkage, such as a disulfide linkage. In some embodiments, the first polypeptide chain and the second polypeptide chain are linked by at least one disulfide bond. In some embodiments, the caTCR further comprises one or more T cell co-stimulatory signaling sequences. The one or more co-stimulatory signaling sequences can be, individually, all or a portion of the intracellular domain of a co-stimulatory molecule including, for example, CD27, CD28, 4-1BB (CD137), OX40, CD30, CD40, ICOS, lymphocyte function-associated antigen-1 (LFA-1), CD2, CD7, LIGHT, NKG2C, B7-H3, a ligand that specifically binds with CD83, and the like. In some embodiments, the one or more co-stimulatory signaling sequences are between the first TCR-TM and the first TCR intracellular domain and/or between the second TCR-TM and the second TCR intracellular domain. In some embodiments, the one or more co-stimulatory signaling sequences are C-terminal to the first TCRD and/or the second TCRD. In some embodiments, the caTCR lacks a T cell co-stimulatory signaling sequence. In some embodiments, the caTCR lacks a functional primary immune cell signaling domain. In some embodiments, the caTCR lacks a functional primary immune cell signaling domain of a TCR-associated T cell activation molecule selected from the group consisting of CD3ζ (TCRζ), FcRγ, FcRβ, CD3γ, CD3δ, CD3ε, CD5, CD22, CD79a, CD79b, and CD66d. In some embodiments, the caTCR lacks any primary immune cell signaling sequences. In some embodiments, the caTCR further comprises a stabilization module comprising a first stabilization domain and a second stabilization domain, wherein the first and second stabilization domains have a binding affinity for each other that stabilizes the caTCR. In some embodiments, the stabilization module is located between the antigen-binding module and the TCRM. In some embodiments, the stabilization module comprises an antibody constant domain or a TCR constant domain, or a fragment thereof. In some embodiments, the stabilization module comprises a pair of antibody constant domains selected from the group consisting of C_{H1}-C_{L}, C_{H2}-C_{H2}, C_{H3}-C_{H3}, and C_{H4}-C_{H4}, or fragments thereof. In some embodiments, the stabilization module comprises a pair of TCR constant domains selected from the group consisting of Cα-Cβ and Cγ-Cδ, or fragments thereof. In some embodiments, the caTCR further comprises a spacer module between any two caTCR modules or domains. In some embodiments, the spacer module comprises one or more peptide linkers connecting two caTCR modules or domains.

In some embodiments, a caTCR comprises a first polypeptide chain and a second polypeptide chain, in which the first polypeptide chain comprises an antibody V_{H} fused to a first antibody constant domain (e.g., C_{H1}) fused to a transmembrane domain and an intracellular immune cell signaling domain, and the second polypeptide comprises a second antibody V_{L} fused to an antibody constant domain (e.g., C_{L}) fused to a transmembrane domain and an intracellular immune cell signaling domain. In some embodiments, the first and second polypeptide chains are linked, such as by a covalent linkage (e.g., peptide or other chemical linkage) or non-covalent linkage. In some embodiments, the caTCR is a heterodimer comprising the first polypeptide chain and the second polypeptide chain. In some embodiments, the first polypeptide chain and the second polypeptide chain are linked by at least one disulfide bond. A caTCR as used herein is non-naturally occurring. In some embodiments, the first antibody constant domain and the second antibody constant domain are selected from the group consisting of CH1/CL, CH2/CH2, CH3/CH3, and CH4/CH4. In some embodiments, the caTCR does not comprise variable domains of a TCR.

In some embodiments, the caTCR comprises: a) a first polypeptide chain comprising a first antigen-binding domain comprising a V_{H} domain, a first stabilization domain comprising a C_{H}1 domain, and a first T cell receptor domain (TCRD) comprising a first transmembrane domain of a first TCR subunit; and b) a second polypeptide chain comprising a second antigen-binding domain comprising a V_{L} domain, a second stabilization domain comprising a C_{L} domain, and a second TCRD comprising a second transmembrane domain of a second TCR subunit, wherein the V_{H} domain of the first antigen-binding domain and the V_{L} domain of the second antigen-binding domain form an antigen-binding module that specifically binds to a target (e.g., a WTMC complex), wherein the C_{H}1 domain of the first stabilization domain and the C_{L} domain of the second stabilization domain form a stabilization module, and wherein the first TCRD and the second TCRD form a T cell receptor module (TCRM) that is capable of associating with at least one TCR-associated signaling module. In some embodiments, the stabilization module comprises a disulfide bond between a residue in the C_{H}1 domain and a residue in the C_{L} domain. In some embodiments, the C_{H}1 domain is fused to the first TCRD via a first hinge region, and the C_{L} domain is fused to the second TCRD via a second hinge region. In some embodiments, the C_{H}1 domain is fused to the first TCRD via a first peptide linker, and the C_{L} domain is fused to the second TCRd via a second peptide linker.

In some embodiments, the caTCR comprises: a) a first polypeptide chain comprising a first antigen-binding domain comprising a V_{H} antibody domain and a first TCRD comprising a first transmembrane domain of a first TCR subunit; and b) a second polypeptide chain comprising a second antigen-binding domain comprising a V_{L} antibody domains and a second TCRD comprising a second transmembrane domain of a second TCR subunit, wherein the V_{H} domain of the first antigen-binding domain and the V_{L} domain of the second antigen-binding domain form an antigen-binding module, wherein the first TCRD and the second TCRD form a T cell receptor module (TCRM) that is capable of associating with at least one TCR-associated signaling module. In some embodiments, the caTCR does not comprise an antibody constant domain.

In some embodiments, the caTCR comprises: a) a first polypeptide chain comprising a first antigen-binding domain comprising a first V_{H} domain, a first stabilization domain comprising a first C_{H}1 domain, and a first T cell receptor domain (TCRD) comprising a first transmembrane domain of a first TCR subunit; b) a second polypeptide chain comprising a second antigen-binding domain comprising a first V_{L} domain, and a second stabilization domain comprising a first C_{L} domain; c) a third polypeptide chain comprising a third antigen-binding domain comprising a second V_{H} domain. a third stabilization domain comprising a second C_{H}1 domain, and a second TCRD comprising a second transmembrane domain of a second TCR subunit; and d) a fourth polypeptide chain comprising a fourth antigen-binding domain comprising a second V_{L} domain, and a fourth stabilization domain comprising a second C_{L} domain, wherein the first V_{H} domain of the first antigen-binding domain and the first V_{L} domain of the second antigen-binding domain form a first antigen-binding module that specifically binds to a first target, wherein the second V_{H} domain of the third antigen-binding domain and the second V_{L} domain of the fourth antigen-binding domain form a second antigen-binding module that specifically binds to a second target, wherein the first C_{H}1 domain of the first stabilization domain and the first C_{L} domain of the second stabilization domain form a first stabilization module, wherein the second C_{H}1 domain of the third stabilization domain and the second C_{L} domain of the fourth stabilization domain form a second stabilization module, and wherein the first TCRD and the second TCRD form a T cell receptor module (TCRM) that is capable of associating with at least one TCR-associated signaling module. In some embodiments, the first C_{H}1 domain is fused to the first TCRD via a first hinge region, and the second C_{H}1 domain is fused to the second TCRD via a second hinge region.

In some embodiments, the caTCR comprises: a) a first polypeptide chain comprising a first antigen-binding domain comprising an scFv domain and a first TCRD comprising a first transmembrane domain of a first TCR subunit; and b) a second polypeptide chain comprising a second TCRD comprising a second transmembrane domain of a second TCR subunit, wherein the first TCRD and the second TCRD form a T cell receptor module (TCRM) that is capable of associating with at least one TCR-associated signaling module.

In some embodiments, the caTCR comprises: a) a first polypeptide chain comprising a first antigen-binding domain comprising a first scFv domain and a first TCRD comprising a first transmembrane domain of a first TCR subunit; and b) a second polypeptide chain comprising a second scFv domain and a second TCRD comprising a second transmembrane domain of a second TCR subunit, wherein the first TCRD and the second TCRD form a T cell receptor module (TCRM) that is capable of associating with at least one TCR-associated signaling module. In some embodiments, the first scFv and the second scFv bind to the same target. In some embodiments, the first scFv and the second scFv bind to different targets.

In some embodiments, the caTCR comprises a TCRM that comprises a) a first T cell receptor domain (TCRD) comprising a first TCR transmembrane domain (TCR-TM) and b) a second TCRD comprising a second TCR-TM, wherein the TCRM is capable of associating with at least one TCR-associated signaling molecule. In some embodiments, both of the TCR-TMs are naturally occurring. In some embodiments, at least one of the TCR-TMs is non-naturally occurring. In some embodiments, both of the TCR-TMs are non-naturally occurring. In some embodiments, the first TCR-TM is derived from one of the transmembrane domains of a T cell receptor (such as an αβ TCR or a γδ TCR) and the second TCR-TM is derived from the other transmembrane domain of the T cell receptor. In some embodiments, the TCRM allows for enhanced association with the at least one TCR-associated signaling molecule as compared to a TCRM comprising the transmembrane domains of the T cell receptor. Association or recruitment of TCR-associated signaling molecules can be determined by methods known in the art, such as FACS analysis for TCR-CD3 complex surface expression or co-immunoprecipitation of CD3 subunits with the caTCR.

In some embodiments, the caTCR comprises an antigen-binding module that comprises a first antigen-binding domain comprising a V_{H} antibody domain (e.g., a V_{H} antibody domain described herein) and a second antigen-binding domain comprising a V_{L} antibody domain (e.g., a V_{L} antibody domain described herein). In some embodiments, the V_{H} antibody domain and V_{L} antibody domain CDRs are derived from the same anti-WTMC antibody moiety. In some embodiments, some of the V_{H} antibody domain and V_{L} antibody domain CDRs are derived from different antibody moieties. In some embodiments, the V_{H} antibody domain and/or V_{L} antibody domain are human, humanized, chimeric, semi-synthetic, or fully synthetic.

In some embodiments, the caTCR comprises an antigen-binding module linked to a TCRM described herein, optionally including a stabilization module. For example, in some embodiments, the caTCR comprises the antigen-binding module linked to the N-terminus of one or both of the TCRDs. In some embodiments, the caTCR comprises a stabilization module between a TCRM and an antigen-binding module. In some embodiments, the caTCR further comprises a spacer module between any two caTCR modules or domains. In some embodiments, the spacer module comprises one or more peptide linkers between about 5 to about 70 (such as about any of 5, 10, 15, 20, 25, 30, 35, 40, 45, 50, 55, 60, 65, or 70, including any ranges between these values) amino acids in length. In some embodiments, the caTCR further comprises one or more accessory intracellular domains. In some embodiments, the one or more accessory intracellular domains are carboxy-terminal to the first and/or second TCRD. In some embodiments, the one or more accessory intracellular domains are between the first TCR-TM and the first TCR intracellular domain and/or between the second TCR-TM and the second TCR intracellular domain. In some embodiments, the one or more accessory intracellular domains comprise, individually, a T-cell costimulatory domain. In some embodiments, the T-cell costimulatory domain comprises all or a portion of the intracellular domain of an immune co-stimulatory molecule (such as CD27, CD28, 4-1BB (CD137), OX40, CD30, CD40, ICOS, lymphocyte function-associated antigen-1 (LFA-1), CD2, CD7, LIGHT, NKG2C, B7-H3, a ligand that specifically binds with CD83, and the like).

In some embodiments, the caTCR comprises a) an antigen-binding module comprising an antibody moiety, and b) a T cell receptor module (TCRM) comprising a first TCR domain (TCRD) comprising a first TCR transmembrane domain (TCR-TM) derived from one of the transmembrane domains of a naturally occurring TCR (such as an aPTCR or a γδTCR) and a second TCRD comprising a second TCR-TM derived from the other transmembrane domain of the naturally occurring TCR (such as an αβTCR or a γδTCR), wherein the TCRM is capable of associating with at least one TCR-associated signaling molecule (such as CD3δε, CD3γε, and/or CD3ζζ), and wherein the antibody moiety is linked to the first and/or second TCRDs.

In some embodiments, the anti-WTMC construct is a chimeric antibody-T cell receptor construct (caTCR) comprising an anti-WTMC antibody moiety (such as any one of the anti-WTMC antibody moieties described in the “Anti-WTMC Antibody Moieties” section). Such construct is also referred to herein as an “anti-WTMC caTCR.” In some embodiments, the anti-WTMC caTCR specifically bind to a WTMC complex and is capable of associating with at least one TCR-associated signaling molecule (such as CD3δ8, CD37F, and/or CD3ζ). In some embodiments, the caTCR does not comprise a constant domain of a TCR.

In some embodiments, a caTCR comprises a first polypeptide chain and a second polypeptide chain, in which the first polypeptide chain comprises an antibody V_{H} fused to an antibody CH1 fused to a transmembrane domain and an intracellular immune cell signaling domain, and the second polypeptide comprises an antibody V_{L} fused to an antibody C_{L} fused to a transmembrane domain and an intracellular immune cell signaling domain. In some embodiments, the first and second polypeptide chains are linked, such as by a covalent linkage (e.g., peptide or other chemical linkage) or non-covalent linkage. In some embodiments, the caTCR is a heterodimer comprising the first polypeptide chain and the second polypeptide chain. In some embodiments, the first polypeptide chain and the second polypeptide chain are linked by at least one disulfide bond. The specificity of the anti-WTMC caTCR derives from an antibody moiety that confers binding specificity to a complex comprising a WT1 peptide (e.g., WT1-RMF) (SEQ ID NO: 113) and a MHC class I protein. In some embodiments, the antibody moiety specifically recognizes amino acid residues FPNA (SEQ ID NO: 349) of SEQ ID NO: 113. In some embodiments, the MHC class I protein is HLA-A*02:01. A caTCR as used herein is non-naturally occurring.

In some embodiments, the caTCR comprises a target-binding domain (e.g., an antibody moiety) that specifically binds a WTMC. In some embodiments, the caTCR comprises a target-binding domain (e.g., an antibody moiety) that does not specifically bind a WTMC. In some embodiments, the caTCR comprises two or more target-binding domains (e.g., antibody moieties) that specifically bind to the same or different targets. In some embodiments, the caTCR is monospecific. In some embodiments, the caTCR is multispecific, such as bispecific.

In some embodiments, the target-binding domain is an antibody moiety, such as a Fab, a Fab′, a (Fab′)2, an Fv, or a single chain Fv (scFv). In some embodiments, the target-binding domain is an extracellular domain of a receptor or a ligand. In some embodiments, the target-binding domain specifically binds a cell surface protein. In some embodiments, the target-binding domain specifically binds a complex comprising a peptide and an MHC molecule.

In some embodiments, the target-binding domain specifically binds a target expressed or present on a cancer cell. In some embodiments, the target-binding domain specifically binds a target expressed or present on a leukemia cell. In some embodiments, the target-binding domain specifically binds CD33, CD371, CD123, and/or CD15. In some embodiments, the target-binding domain specifically binds a peptide/MHC complex, wherein the peptide is WT1. In some embodiments, the target-binding domain specifically binds a target expressed or present on a cancer type.

In some embodiments, the anti-WTMC caTCR comprises any one of the anti-WTMC antibody moieties described herein. In some embodiments, the anti-WTMC caTCR comprises an anti-WTMC antibody moiety comprising (a) a HC-CDR1 comprising an amino acid sequence set forth in SEQ ID NO: 1; (b) a HC-CDR2 comprising an amino acid sequence set forth in SEQ ID NO: 2; (c) a HC-CDR3 comprising an amino acid sequence set forth in SEQ ID NO: 3; (d) a LC-CDR1 comprising an amino acid sequence set forth in SEQ ID NO: 4; (e) a LC-CDR2 comprising an amino acid sequence set forth in SEQ ID NO: 5; and (f) a LC-CDR3 comprising an amino acid sequence set forth in SEQ ID NO: 6. In some embodiments, the CDRs are human CDRs. In some embodiments, the anti-WTMC caTCR comprises an anti-WTMC antibody moiety comprising one, two, or three CDRs of a V_{H} domain comprising SEQ ID NO: 85 and one, two, or three CDRs of a V_{L} domain comprising SEQ ID NO: 86. In some embodiments, the anti-WTMC caTCR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 85 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 86. In some embodiments, the anti-WTMC caTCR comprises an anti-WTMC antibody moiety comprising a V_{H} domain comprising SEQ ID NO: 85 and a V_{L} domain comprising SEQ ID NO: 86.

In some embodiments, the anti-WTMC caTCR comprises any one of the anti-WTMC antibody moieties described herein. In some embodiments, the anti-WTMC caTCR comprises an anti-WTMC antibody moiety comprising (a) a HC-CDR1 comprising an amino acid sequence set forth in SEQ ID NO: 7; (b) a HC-CDR2 comprising an amino acid sequence set forth in SEQ ID NO: 8; (c) a HC-CDR3 comprising an amino acid sequence set forth in SEQ ID NO: 9; (d) a LC-CDR1 comprising an amino acid sequence set forth in SEQ ID NO: 10; (e) a LC-CDR2 comprising an amino acid sequence set forth in SEQ ID NO: 11; and (f) a LC-CDR3 comprising an amino acid sequence set forth in SEQ ID NO: 12. In some embodiments, the CDRs are human CDRs. In some embodiments, the anti-WTMC caTCR comprises an anti-WTMC antibody moiety comprising one, two, or three CDRs of a V_{H} domain comprising SEQ ID NO: 87 and one, two, or three CDRs of a V_{L} domain comprising SEQ ID NO: 88. In some embodiments, the anti-WTMC caTCR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 87 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 88. In some embodiments, the anti-WTMC caTCR comprises an anti-WTMC antibody moiety comprising a V_{H} domain comprising SEQ ID NO: 87 and a V_{L} domain comprising SEQ ID NO: 88.

In some embodiments, the anti-WTMC caTCR comprises any one of the anti-WTMC antibody moieties described herein. In some embodiments, the anti-WTMC caTCR comprises an anti-WTMC antibody moiety comprising (a) a HC-CDR1 comprising an amino acid sequence set forth in SEQ ID NO: 13; (b) a HC-CDR2 comprising an amino acid sequence set forth in SEQ ID NO: 14; (c) a HC-CDR3 comprising an amino acid sequence set forth in SEQ ID NO: 15; (d) a LC-CDR1 comprising an amino acid sequence set forth in SEQ ID NO: 16; (e) a LC-CDR2 comprising an amino acid sequence set forth in SEQ ID NO: 17; and (f) a LC-CDR3 comprising an amino acid sequence set forth in SEQ ID NO: 18. In some embodiments, the CDRs are human CDRs. In some embodiments, the anti-WTMC caTCR comprises an anti-WTMC antibody moiety comprising one, two, or three CDRs of a V_{H} domain comprising SEQ ID NO: 89 and one, two, or three CDRs of a V_{L} domain comprising SEQ ID NO: 90. In some embodiments, the anti-WTMC caTCR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 89 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 90. In some embodiments, the anti-WTMC caTCR comprises an anti-WTMC antibody moiety comprising a V_{H} domain comprising SEQ ID NO: 89 and a V_{L} domain comprising SEQ ID NO: 90.

In some embodiments, the anti-WTMC caTCR comprises any one of the anti-WTMC antibody moieties described herein. In some embodiments, the anti-WTMC caTCR comprises an anti-WTMC antibody moiety comprising (a) a HC-CDR1 comprising an amino acid sequence set forth in SEQ ID NO: 19; (b) a HC-CDR2 comprising an amino acid sequence set forth in SEQ ID NO: 20; (c) a HC-CDR3 comprising an amino acid sequence set forth in SEQ ID NO: 21; (d) a LC-CDR1 comprising an amino acid sequence set forth in SEQ ID NO: 22; (e) a LC-CDR2 comprising an amino acid sequence set forth in SEQ ID NO: 23; and (f) a LC-CDR3 comprising an amino acid sequence set forth in SEQ ID NO: 24. In some embodiments, the CDRs are human CDRs. In some embodiments, the anti-WTMC caTCR comprises an anti-WTMC antibody moiety comprising one, two, or three CDRs of a V_{H} domain comprising SEQ ID NO: 91 and one, two, or three CDRs of a V_{L} domain comprising SEQ ID NO: 92. In some embodiments, the anti-WTMC caTCR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 91 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 92. In some embodiments, the anti-WTMC caTCR comprises an anti-WTMC antibody moiety comprising a V_{H} domain comprising SEQ ID NO: 91 and a V_{L} domain comprising SEQ ID NO: 92.

In some embodiments, the anti-WTMC caTCR comprises any one of the anti-WTMC antibody moieties described herein. In some embodiments, the anti-WTMC caTCR comprises an anti-WTMC antibody moiety comprising (a) a HC-CDR1 comprising an amino acid sequence set forth in SEQ ID NO: 25; (b) a HC-CDR2 comprising an amino acid sequence set forth in SEQ ID NO: 26; (c) a HC-CDR3 comprising an amino acid sequence set forth in SEQ ID NO: 27; (d) a LC-CDR1 comprising an amino acid sequence set forth in SEQ ID NO: 28; (e) a LC-CDR2 comprising an amino acid sequence set forth in SEQ ID NO: 29; and (f) a LC-CDR3 comprising an amino acid sequence set forth in SEQ ID NO: 30. In some embodiments, the CDRs are human CDRs. In some embodiments, the anti-WTMC caTCR comprises an anti-WTMC antibody moiety comprising one, two, or three CDRs of a V_{H} domain comprising SEQ ID NO: 93 and one, two, or three CDRs of a V_{L} domain comprising SEQ ID NO: 94. In some embodiments, the anti-WTMC caTCR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 93 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 94. In some embodiments, the anti-WTMC caTCR comprises an anti-WTMC antibody moiety comprising a V_{H} domain comprising SEQ ID NO: 93 and a V_{L} domain comprising SEQ ID NO: 94.

In some embodiments, the anti-WTMC caTCR comprises any one of the anti-WTMC antibody moieties described herein. In some embodiments, the anti-WTMC caTCR comprises an anti-WTMC antibody moiety comprising (a) a HC-CDR1 comprising an amino acid sequence set forth in SEQ ID NO: 31; (b) a HC-CDR2 comprising an amino acid sequence set forth in SEQ ID NO: 32; (c) a HC-CDR3 comprising an amino acid sequence set forth in SEQ ID NO: 33; (d) a LC-CDR1 comprising an amino acid sequence set forth in SEQ ID NO: 34; (e) a LC-CDR2 comprising an amino acid sequence set forth in SEQ ID NO: 35; and (f) a LC-CDR3 comprising an amino acid sequence set forth in SEQ ID NO: 36. In some embodiments, the CDRs are human CDRs. In some embodiments, the anti-WTMC caTCR comprises an anti-WTMC antibody moiety comprising one, two, or three CDRs of a V_{H} domain comprising SEQ ID NO: 95 and one, two, or three CDRs of a V_{L} domain comprising SEQ ID NO: 96. In some embodiments, the anti-WTMC caTCR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 95 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 96. In some embodiments, the anti-WTMC caTCR comprises an anti-WTMC antibody moiety comprising a V_{H} domain comprising SEQ ID NO: 95 and a V_{L} domain comprising SEQ ID NO: 96.

In some embodiments, the anti-WTMC caTCR comprises any one of the anti-WTMC antibody moieties described herein. In some embodiments, the anti-WTMC caTCR comprises an anti-WTMC antibody moiety comprising (a) a HC-CDR1 comprising an amino acid sequence set forth in SEQ ID NO: 37; (b) a HC-CDR2 comprising an amino acid sequence set forth in SEQ ID NO: 38; (c) a HC-CDR3 comprising an amino acid sequence set forth in SEQ ID NO: 39; (d) a LC-CDR1 comprising an amino acid sequence set forth in SEQ ID NO: 40; (e) a LC-CDR2 comprising an amino acid sequence set forth in SEQ ID NO: 41; and (f) a LC-CDR3 comprising an amino acid sequence set forth in SEQ ID NO: 42. In some embodiments, the CDRs are human CDRs. In some embodiments, the anti-WTMC caTCR comprises an anti-WTMC antibody moiety comprising one, two, or three CDRs of a V_{H} domain comprising SEQ ID NO: 97 and one, two, or three CDRs of a V_{L} domain comprising SEQ ID NO: 98. In some embodiments, the anti-WTMC caTCR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 97 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 98. In some embodiments, the anti-WTMC caTCR comprises an anti-WTMC antibody moiety comprising a V_{H} domain comprising SEQ ID NO: 97 and a V_{L} domain comprising SEQ ID NO: 98.

In some embodiments, the anti-WTMC caTCR comprises any one of the anti-WTMC antibody moieties described herein. In some embodiments, the anti-WTMC caTCR comprises an anti-WTMC antibody moiety comprising (a) a HC-CDR1 comprising an amino acid sequence set forth in SEQ ID NO: 43; (b) a HC-CDR2 comprising an amino acid sequence set forth in SEQ ID NO: 44; (c) a HC-CDR3 comprising an amino acid sequence set forth in SEQ ID NO: 45; (d) a LC-CDR1 comprising an amino acid sequence set forth in SEQ ID NO: 46; (e) a LC-CDR2 comprising an amino acid sequence set forth in SEQ ID NO: 47; and (f) a LC-CDR3 comprising an amino acid sequence set forth in SEQ ID NO: 48. In some embodiments, the CDRs are human CDRs. In some embodiments, the anti-WTMC caTCR comprises an anti-WTMC antibody moiety comprising one, two, or three CDRs of a V_{H} domain comprising SEQ ID NO: 99 and one, two, or three CDRs of a V_{L} domain comprising SEQ ID NO: 100. In some embodiments, the anti-WTMC caTCR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 99 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 100. In some embodiments, the anti-WTMC caTCR comprises an anti-WTMC antibody moiety comprising a V_{H} domain comprising SEQ ID NO: 99 and a V_{L} domain comprising SEQ ID NO: 100.

In some embodiments, the anti-WTMC caTCR comprises any one of the anti-WTMC antibody moieties described herein. In some embodiments, the anti-WTMC caTCR comprises an anti-WTMC antibody moiety comprising (a) a HC-CDR1 comprising an amino acid sequence set forth in SEQ ID NO: 49; (b) a HC-CDR2 comprising an amino acid sequence set forth in SEQ ID NO: 50; (c) a HC-CDR3 comprising an amino acid sequence set forth in SEQ ID NO: 51; (d) a LC-CDR1 comprising an amino acid sequence set forth in SEQ ID NO: 52; (e) a LC-CDR2 comprising an amino acid sequence set forth in SEQ ID NO: 53; and (f) a LC-CDR3 comprising an amino acid sequence set forth in SEQ ID NO: 54. In some embodiments, the CDRs are human CDRs. In some embodiments, the anti-WTMC caTCR comprises an anti-WTMC antibody moiety comprising one, two, or three CDRs of a V_{H} domain comprising SEQ ID NO: 101 and one, two, or three CDRs of a V_{L} domain comprising SEQ ID NO: 102. In some embodiments, the anti-WTMC caTCR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 101 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 102. In some embodiments, the anti-WTMC caTCR comprises an anti-WTMC antibody moiety comprising a V_{H} domain comprising SEQ ID NO: 101 and a V_{L} domain comprising SEQ ID NO: 102.

In some embodiments, the anti-WTMC caTCR comprises any one of the anti-WTMC antibody moieties described herein. In some embodiments, the anti-WTMC caTCR comprises an anti-WTMC antibody moiety comprising (a) a HC-CDR1 comprising an amino acid sequence set forth in SEQ ID NO: 55; (b) a HC-CDR2 comprising an amino acid sequence set forth in SEQ ID NO: 56; (c) a HC-CDR3 comprising an amino acid sequence set forth in SEQ ID NO: 57; (d) a LC-CDR1 comprising an amino acid sequence set forth in SEQ ID NO: 58; (e) a LC-CDR2 comprising an amino acid sequence set forth in SEQ ID NO: 59; and (f) a LC-CDR3 comprising an amino acid sequence set forth in SEQ ID NO: 60. In some embodiments, the CDRs are human CDRs. In some embodiments, the anti-WTMC caTCR comprises an anti-WTMC antibody moiety comprising one, two, or three CDRs of a V_{H} domain comprising SEQ ID NO: 103 and one, two, or three CDRs of a V_{L} domain comprising SEQ ID NO: 104. In some embodiments, the anti-WTMC caTCR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 103 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 104. In some embodiments, the anti-WTMC caTCR comprises an anti-WTMC antibody moiety comprising a V_{H} domain comprising SEQ ID NO: 103 and a V_{L} domain comprising SEQ ID NO: 104.

In some embodiments, the anti-WTMC caTCR comprises any one of the anti-WTMC antibody moieties described herein. In some embodiments, the anti-WTMC caTCR comprises an anti-WTMC antibody moiety comprising (a) a HC-CDR1 comprising an amino acid sequence set forth in SEQ ID NO: 61; (b) a HC-CDR2 comprising an amino acid sequence set forth in SEQ ID NO: 62; (c) a HC-CDR3 comprising an amino acid sequence set forth in SEQ ID NO: 63; (d) a LC-CDR1 comprising an amino acid sequence set forth in SEQ ID NO: 64; (e) a LC-CDR2 comprising an amino acid sequence set forth in SEQ ID NO: 65; and (f) a LC-CDR3 comprising an amino acid sequence set forth in SEQ ID NO: 66. In some embodiments, the CDRs are human CDRs. In some embodiments, the anti-WTMC caTCR comprises an anti-WTMC antibody moiety comprising one, two, or three CDRs of a V_{H} domain comprising SEQ ID NO: 105 and one, two, or three CDRs of a V_{L} domain comprising SEQ ID NO: 106. In some embodiments, the anti-WTMC caTCR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 105 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 106. In some embodiments, the anti-WTMC caTCR comprises an anti-WTMC antibody moiety comprising a V_{H} domain comprising SEQ ID NO: 105 and a V_{L} domain comprising SEQ ID NO: 106.

In some embodiments, the anti-WTMC caTCR comprises any one of the anti-WTMC antibody moieties described herein. In some embodiments, the anti-WTMC caTCR comprises an anti-WTMC antibody moiety comprising (a) a HC-CDR1 comprising an amino acid sequence set forth in SEQ ID NO: 67; (b) a HC-CDR2 comprising an amino acid sequence set forth in SEQ ID NO: 68; (c) a HC-CDR3 comprising an amino acid sequence set forth in SEQ ID NO: 69; (d) a LC-CDR1 comprising an amino acid sequence set forth in SEQ ID NO: 70; (e) a LC-CDR2 comprising an amino acid sequence set forth in SEQ ID NO: 71; and (f) a LC-CDR3 comprising an amino acid sequence set forth in SEQ ID NO: 72. In some embodiments, the CDRs are human CDRs. In some embodiments, the anti-WTMC caTCR comprises an anti-WTMC antibody moiety comprising one, two, or three CDRs of a V_{H} domain comprising SEQ ID NO: 107 and one, two, or three CDRs of a V_{L} domain comprising SEQ ID NO: 108. In some embodiments, the anti-WTMC caTCR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 107 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 108. In some embodiments, the anti-WTMC caTCR comprises an anti-WTMC antibody moiety comprising a V_{H} domain comprising SEQ ID NO: 107 and a V_{L} domain comprising SEQ ID NO: 108.

In some embodiments, the anti-WTMC caTCR comprises any one of the anti-WTMC antibody moieties described herein. In some embodiments, the anti-WTMC caTCR comprises an anti-WTMC antibody moiety comprising (a) a HC-CDR1 comprising an amino acid sequence set forth in SEQ ID NO: 73; (b) a HC-CDR2 comprising an amino acid sequence set forth in SEQ ID NO: 74; (c) a HC-CDR3 comprising an amino acid sequence set forth in SEQ ID NO: 75; (d) a LC-CDR1 comprising an amino acid sequence set forth in SEQ ID NO: 76; (e) a LC-CDR2 comprising an amino acid sequence set forth in SEQ ID NO: 77; and (f) a LC-CDR3 comprising an amino acid sequence set forth in SEQ ID NO: 78. In some embodiments, the CDRs are human CDRs. In some embodiments, the anti-WTMC caTCR comprises an anti-WTMC antibody moiety comprising one, two, or three CDRs of a V_{H} domain comprising SEQ ID NO: 109 and one, two, or three CDRs of a V_{L} domain comprising SEQ ID NO: 110. In some embodiments, the anti-WTMC caTCR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 109 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 110. In some embodiments, the anti-WTMC caTCR comprises an anti-WTMC antibody moiety comprising a V_{H} domain comprising SEQ ID NO: 109 and a V_{L} domain comprising SEQ ID NO: 110.

In some embodiments, the anti-WTMC caTCR comprises any one of the anti-WTMC antibody moieties described herein. In some embodiments, the anti-WTMC caTCR comprises an anti-WTMC antibody moiety comprising (a) a HC-CDR1 comprising an amino acid sequence set forth in SEQ ID NO: 79; (b) a HC-CDR2 comprising an amino acid sequence set forth in SEQ ID NO: 80; (c) a HC-CDR3 comprising an amino acid sequence set forth in SEQ ID NO: 81; (d) a LC-CDR1 comprising an amino acid sequence set forth in SEQ ID NO: 82; (e) a LC-CDR2 comprising an amino acid sequence set forth in SEQ ID NO: 83; and (f) a LC-CDR3 comprising an amino acid sequence set forth in SEQ ID NO: 84. In some embodiments, the CDRs are human CDRs. In some embodiments, the anti-WTMC caTCR comprises an anti-WTMC antibody moiety comprising one, two, or three CDRs of a V_{H} domain comprising SEQ ID NO: 111 and one, two, or three CDRs of a V_{L} domain comprising SEQ ID NO: 112. In some embodiments, the anti-WTMC caTCR comprises an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 111 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 112. In some embodiments, the anti-WTMC caTCR comprises an anti-WTMC antibody moiety comprising a V_{H} domain comprising SEQ ID NO: 111 and a V_{L} domain comprising SEQ ID NO: 112.

In some embodiments, the anti-WTMC caTCR comprises the anti-WTMC antibody moiety of Clone 1, 2, 10, 11, 12, 14, 15, 17, 18, 26, 30, 32, 34, or 36. In some embodiments, the anti-WTMC caTCR comprises the anti-WTMC antibody moiety of Clone 18 or Clone 34.

### Chimeric Stimulatory Receptor Constructs (CSRs)

Also provided herein are WTMC-specific chimeric stimulatory receptor constructs, which are alternatively referred to herein as chimeric signaling receptor constructs (i.e., “anti-WTMC CSRs”). In some embodiments, there is provided WTMC-specific chimeric stimulatory receptor constructs, which are alternatively referred to herein as chimeric signaling receptor constructs (i.e., “anti-WTMC CSRs”), comprising an anti-WTMC antibody moiety (such as any one of the anti-WTMC antibody moieties described in the “Anti-WTMC Antibody Moieties” section).

The features of CSRs described in this section apply to both anti-WTMC CSRs and CSRs that do not target WTMC.

Exemplary CSRs are discussed in US 2021/0107976, the contents of which are incorporated herein by reference in their entirety. In some embodiments, the CSR is expressed on the surface of an immune cell (such as a T cell). The CSR binds to a target, and upon binding to the target, is capable of stimulating the immune cell on which the CSR is expressed. In some embodiments, the CSR is a bispecific CSR. In some embodiments, the CSR comprises a target-binding module, a transmembrane (TM) module, and a co-stimulatory immune cell signaling module that allows for stimulating the immune cell in or on which the CSR is expressed. In some embodiments, the CSR lacks a functional primary immune cell signaling sequence. In some embodiments, the CSR lacks a primary immune cell signaling sequence. In some embodiments, the CSR comprises a single polypeptide chain comprising the target-binding module, transmembrane module, and co-stimulatory signaling module. In some embodiments, the CSR comprises a first polypeptide chain and a second polypeptide chain, wherein the first and second polypeptide chains together form the target-binding module, the transmembrane module, and the co-stimulatory signaling module. In some embodiments, the first and second polypeptide chains are separate polypeptide chains, and the CSR is a multimer, such as a dimer. In some embodiments, the first and second polypeptide chains are covalently linked, such as by a peptide linkage, or by another chemical linkage, such as a disulfide linkage. In some embodiments, the first polypeptide chain and the second polypeptide chain are linked by at least one disulfide bond.

Also provided are effector cells (such as T cells) expressing a CSR of the present disclosure. Such effector cells (such as T cells) are produced by introducing (e.g., transducing or transfecting) a nucleic acid encoding a CSR described herein (or a vector comprising such a nucleic acid) into the effector cell (e.g., T cell).

Examples of co-stimulatory immune cell signaling domains for use in an CSR include, but are not limited to, the cytoplasmic sequences of co-receptors of the T cell receptor, which can act in concert with a caTCR to initiate signal transduction following caTCR engagement, as well as any derivative or variant of these sequences and any synthetic sequence that has the same functional capability. Thus, in some embodiments provided is an effector cell (such as a T cell) that expresses a caTCR and a CSR. Effector cells (such as T cells) expressing a caTCR and a CSR (i.e., “caTCR plus CSR effector cells”) are described in further detail below.

It is known that signals generated through the TCR alone are insufficient for full activation of the T cell and that a secondary or co-stimulatory signal is also required. Thus, T cell activation can be said to be mediated by two distinct classes of intracellular (IC) signaling sequence: those that initiate antigen-dependent primary activation through the TCR (referred to herein as “primary T cell signaling sequences”) and those that act in an antigen-independent manner to provide a secondary or co-stimulatory signal (referred to herein as “co-stimulatory T cell signaling sequences”).

As used herein, “primary immune cell signaling sequences” refer to sequences that act in a stimulatory manner may contain signaling motifs which are known as immunoreceptor tyrosine-based activation motifs or ITAMs. Examples of ITAM-containing primary immune cell signaling sequences include those derived from CD3ζ (TCRζ), FcRγ, FcRβ, CD3γ, CD3δ, CD3ε, CD5, CD79a, CD79b, and CD66d. A “functional” primary immune cell signaling sequence is a sequence that is capable of transducing an immune cell activation signal when operably coupled to an appropriate receptor. “Non-functional” primary immune cell signaling sequences, which may comprise fragments or variants of primary immune cell signaling sequences, are unable to transduce an immune cell activation signal. Thus, in some embodiments, a CSR as described herein lacks a functional primary immune cell signaling sequence, such as a functional signaling sequence comprising an ITAM. In some embodiments, the CSR described herein lack any primary immune cell signaling sequence.

In some embodiments, the CSR comprises a co-stimulatory signaling module that comprises (such as consists of or consists essentially of) all or a portion of the intracellular (IC) signaling domain of an immune cell co-stimulatory molecule including, for example, CD27, CD28, 4-1BB (CD137), OX40, CD30, CD40, ICOS, lymphocyte function-associated antigen-1 (LFA-1), CD2, CD7, LIGHT, NKG2C, B7-H3, a ligand that specifically binds with CD83, and the like. In some embodiments, the CSR comprises a fragment of an immune cell co-stimulatory molecule (fCSM), wherein the fCSM comprises the CSR transmembrane (TM) domain and CSR intracellular (IC) co-stimulatory signaling domain. Exemplary IC co-stimulatory immune cell signaling module sequences and signaling module sequence plus TM domains are provided below:

4-1BB IC signaling sequence:
(SEQ ID NO: 281)
KRGRKKLLYIFKQPFMRPVQTTQEEDGCSCRFPEEEEGGCEL
CD27 IC signaling sequence:
(SEQ ID NO: 282)
QRRKYRSNKGESPVEPAEPCRYSCPREEEGSTIPIQEDYRKPEPACSP
CD28 IC signaling sequence:
(SEQ ID NO: 283)
RSKRSRLLHSDYMNMTPRRPGPTRKHYQPYAPPRDFAAYRS
CD30 IC signaling sequence:
(SEQ ID NO: 284)
HRRACRKRIRQKLHLCYPVQTSQPKLELVDSRPRRSSTQLRSGASVTEPVAEERGL
MSQPLMETCHSVGAAYLESLPLQDASPAGGPSSPRDLPEPRVSTEHTNNKIEKIYIM
KADTVIVGTVKAELPEGRGLAGPAEPELEEELEADHTPHYPEQETEPPLGSCSDVML
SVEEEGKEDPLPTAASGK
OX40 IC signaling sequence:
(SEQ ID NO: 285)
ALYLLRRDQRLPPDAHKPPGGGSFRTPIQEEQADAHSTLAKI
myc tag + truncated CD28 sequence:
(SEQ ID NO: 291)
EQKLISEEDLAAAIEVMYPPPYLDNEKSNGTIIHVKGKHLCPSPLFPGPSKPFWVLV
VVGGVLACYSLLVTVAFIIFWVRSKRSRLLHSDYMNMTPRRPGPTRKHYQPYAPPR
DFAAYRS
truncated CD28 sequence:
(SEQ ID NO: 290)
IEVMYPPPYLDNEKSNGTIIHVKGKHLCPSPLFPGPSKPFWVLVVVGGVLACYSLLV
TVAFIIFWVRSKRSRLLHSDYMNMTPRRPGPTRKHYQPYAPPRDFAAYRS
myc tag + truncated 4-1BB sequence:
(SEQ ID NO: 292)
EQKLISEEDLAAATGPADLSPGASSVTPPAPAREPGHSPQIISFFLALTSTALLFLLFF
LTLRFSVVKRGRKKLLYIFKQPFMRPVQTTQEEDGCSCRFPEEEEGGCEL
truncated 4-1BB sequence:
(SEQ ID NO: 293)
AAATGPADLSPGASSVTPPAPAREPGHSPQIISFFLALTSTALLFLLFFLTLRFSVVKR
GRKKLLYIFKQPFMRPVQTTQEEDGCSCRFPEEEEGGCEL
myc tag + truncated CD27 sequence:
(SEQ ID NO: 294)
EQKLISEEDLAAATGPTHLPYVSEMLEARTAGHMQTLADFRQLPARTLSTHWPPQ
RSLCSSDFIRILVIFSGMFLVFTLAGALFLHQRRKYRSNKGESPVEPAEPCRYSCPRE
EEGSTIPIQEDYRKPEPACSP
truncated CD27 sequence:
(SEQ ID NO: 295)
AAATGPTHLPYVSEMLEARTAGHMQTLADFRQLPARTLSTHWPPQRSLCSSDFIRIL
VIFSGMFLVFTLAGALFLHQRRKYRSNKGESPVEPAEPCRYSCPREEEGSTIPIQEDY
RKPEPACSP
myc tag + truncated CD30 sequence:
(SEQ ID NO: 296)
EQKLISEEDLAAATGAPPLGTQPDCNPTPENGEAPASTSPTQSLLVDSQASKTLPIPT
SAPVALSSTGKPVLDAGPVLFWVILVLVVVVGSSAFLLCHRRACRKRIRQKLHLCY
PVQTSQPKLELVDSRPRRSSTQLRSGASVTEPVAEERGLMSQPLMETCHSVGAAYL
ESLPLQDASPAGGPSSPRDLPEPRVSTEHTNNKIEKIYIMKADTVIVGTVKAELPEGR
GLAGPAEPELEEELEADHTPHYPEQETEPPLGSCSDVMLSVEEEGKEDPLPTAASGK
truncated CD30 sequence:
(SEQ ID NO: 297)
AAATGAPPLGTQPDCNPTPENGEAPASTSPTQSLLVDSQASKTLPIPTSAPVALSSTG
KPVLDAGPVLFWVILVLVVVVGSSAFLLCHRRACRKRIRQKLHLCYPVQTSQPKLE
LVDSRPRRSSTQLRSGASVTEPVAEERGLMSQPLMETCHSVGAAYLESLPLQDASP
AGGPSSPRDLPEPRVSTEHTNNKIEKIYIMKADTVIVGTVKAELPEGRGLAGPAEPEL
EEELEADHTPHYPEQETEPPLGSCSDVMLSVEEEGKEDPLPTAASGK
myc tag + truncated OX40 sequence:
(SEQ ID NO: 298)
EQKLISEEDLAAATGDRDPPATQPQETQGPPARPITVQPTEAWPRTSQGPSTRPVEV
PGGRAVAAILGLGLVLGLLGPLAILLALYLLRRDQRLPPDAHKPPGGGSFRTPIQEE
QADAHSTLAKI
truncated OX40 sequence:
(SEQ ID NO: 299)
AAATGDRDPPATQPQETQGPPARPITVQPTEAWPRTSQGPSTRPVEVPGGRAVAAIL
GLGLVLGLLGPLAILLALYLLRRDQRLPPDAHKPPGGGSFRTPIQEEQADAHSTLAK
I
myc tag + CD8 TM sequence and CD27 IC signaling sequence:
(SEQ ID NO: 300)
EQKLISEEDLAAATGTTTPAPRPPTPAPTIASQPLSLRPEACRPAAGGAVHTRGLDF
ACDIYIWAPLAGTGCVLLLSLVITLYCQRRKYRSNKGESPVEPAEPCRYSCPREEEG
STIPIQEDYRKPEPACSP
CD8 TM sequence and CD27 IC signaling sequence:
(SEQ ID NO: 301)
AAATGTTTPAPRPPTPAPTIASQPLSLRPEACRPAAGGAVHTRGLDFACDIYIWAPL
AGTGCVLLLSLVITLYCQRRKYRSNKGESPVEPAEPCRYSCPREEEGSTIPIQEDYRK
PEPACSP
myc tag + CD8 TM sequence and CD30 IC signaling sequence:
(SEQ ID NO: 302)
EQKLISEEDLAAATGTTTPAPRPPTPAPTIASQPLSLRPEACRPAAGGAVHTRGLDF
ACDIYIWAPLAGTGCVLLLSLVITLYCHRRACRKRIRQKLHLCYPVQTSQPKLELVD
SRPRRSSTQLRSGASVTEPVAEERGLMSQPLMETCHSVGAAYLESLPLQDASPAGG
PSSPRDLPEPRVSTEHTNNKIEKIYIMKADTVIVGTVKAELPEGRGLAGPAEPELEEE
LEADHTPHYPEQETEPPLGSCSDVMLSVEEEGKEDPLPTAASGK
CD8 TM sequence and CD30 IC signaling sequence:
(SEQ ID NO: 303)
AAATGTTTPAPRPPTPAPTIASQPLSLRPEACRPAAGGAVHTRGLDFACDIYIWAPL
AGTGCVLLLSLVITLYCHRRACRKRIRQKLHLCYPVQTSQPKLELVDSRPRRSSTQL
RSGASVTEPVAEERGLMSQPLMETCHSVGAAYLESLPLQDASPAGGPSSPRDLPEPR
VSTEHTNNKIEKIYIMKADTVIVGTVKAELPEGRGLAGPAEPELEEELEADHTPHYP
EQETEPPLGSCSDVMLSVEEEGKEDPLPTAASGK
myc tag + CD8 TM sequence and OX40 IC signaling sequence:
(SEQ ID NO: 304)
EQKLISEEDLAAATGTTTPAPRPPTPAPTIASQPLSLRPEACRPAAGGAVHTRGLDF
ACDIYIWAPLAGTGCVLLLSLVITLYCALYLLRRDQRLPPDAHKPPGGGSFRTPIQEE
QADAHSTLAKI
CD8 TM sequence and OX40 IC signaling sequence:
(SEQ ID NO: 305)
AAATGTTTPAPRPPTPAPTIASQPLSLRPEACRPAAGGAVHTRGLDFACDIYIWAPL
AGTGCVLLLSLVITLYCALYLLRRDQRLPPDAHKPPGGGSFRTPIQEEQADAHSTLA
KI
myc tag + CD8 TM sequence and 4-1BB IC signaling sequence:
(SEQ ID NO: 306)
EQKLISEEDLAAATGTTTPAPRPPTPAPTIASQPLSLRPEACRPAAGGAVHTRGLDF
ACDIYIWAPLAGTGCVLLLSLVITLYCKRGRKKLLYIFKQPFMRPVQTTQEEDGCSC
RFPEEEEGGCEL
CD8 TM sequence and 4-1BB IC signaling sequence:
(SEQ ID NO: 307)
AAATGTTTPAPRPPTPAPTIASQPLSLRPEACRPAAGGAVHTRGLDFACDIYIWAPL
AGTGCVLLLSLVITLYCKRGRKKLLYIFKQPFMRPVQTTQEEDGCSCRFPEEEEGGC
EL
ICOS IC signaling sequence:
(SEQ ID NO: 286)
CWLTKKKYSSSVHDPNGEYMFMRAVNTAKKSRLTDVTL
DAP10 IC signaling sequence:
(SEQ ID NO: 287)
CARPRRSPAQEDGKVYINMPGRG
CD40 IC signaling sequence:
(SEQ ID NO: 288)
KKVAKKPTNKAPHPKQEPQEINFPDDLPGSNTAAPVQETLHGCQPVTQEDGKESRI
SVQERQ

In some embodiments, the CSR comprises an intracellular signaling domain of 4-1BB. In some embodiments, the CSR comprises an intracellular signaling domain of CD27. In some embodiments, the CSR comprises an intracellular signaling domain of CD28. In some embodiments, the CSR comprises an intracellular signaling domain of ICOS. In some embodiments, the CSR comprises an intracellular signaling domain of OX40. In some embodiments, the CSR comprises an intracellular signaling domain comprising an amino acid sequence of any one of SEQ ID NOs: 281-288, and 290-299.

In some embodiments, the transmembrane module of a CSR of the present disclosure comprises one or more transmembrane domains derived from, for example, CD28, CD3ε, CD3ζ, CD45, CD4, CD5, CD8, CD9, CD16, CD22, CD33, CD37, CD64, CD80, CD86, CD134, CD137, or CD154. In some embodiments, the CSR comprises a fragment of a transmembrane protein (fTMP), wherein the fTMP comprises the CSR transmembrane domain. Exemplary transmembrane domain (TM) sequences are provided below:

CD8 TM sequence:
(SEQ ID NO: 308)
IYIWAPLAGTCGVLLLSLVIT
4-1BB TM sequence:
(SEQ ID NO: 309)
IISFFLALTSTALLFLLFFLTLRFSVV
CD27 TM sequence:
(SEQ ID NO: 310)
ILVIFSGMFLVFTLAGALFLH
CD28 TM sequence:
(SEQ ID NO: 311)
FWVLVVVGGVLACYSLLVTVAFIIFWV
CD30 TM sequence:
(SEQ ID NO: 312)
PVLDAGPVLFWVILVLVVVVGSSAFLLC
OX40 TM sequence:
(SEQ ID NO: 313)
VAAILGLGLVLGLLGPLAILL
ICOS TM sequence:
(SEQ ID NO: 314)
FWLPIGCAAFVVCILGCILI
DAP10 TM sequence:
(SEQ ID NO: 315)
LLAGLVAADAVASLLIVGAVFL
CD40 TM sequence:
(SEQ ID NO: 316)
ALVVIPIIFGILFAILLVLVFI

In some embodiments, the CSR comprises a transmembrane domain of CD8. In some embodiments, the CSR comprises a transmembrane domain of 4-1BB. In some embodiments, the CSR comprises a transmembrane domain of CD27. In some embodiments, the CSR comprises a transmembrane domain of CD28. In some embodiments, the CSR comprises a transmembrane domain of CD30. In some embodiments, the CSR comprises a transmembrane domain of OX40. In some embodiments, the CSR comprises a transmembrane domain comprising an amino acid sequence of any one of SEQ ID NOs: 308-316.

In some embodiments, the CSR comprises a transmembrane domain (e.g., CD8 transmembrane domain) and an intracellular signaling domain of CD27. In some embodiments, the CSR comprises a CD8 transmembrane domain and an intracellular signaling domain of CD30. In some embodiments, the CSR comprises a CD8 transmembrane domain and an intracellular signaling domain of OX40. In some embodiments, the CSR comprises a CD8 transmembrane domain and an intracellular signaling domain of 4-1BB. In some embodiments, the CSR comprises a transmembrane domain and an intracellular signaling domain comprising any amino acid sequence of any one of SEQ ID NOs: 300-307.

In some embodiments, the CSR further comprises a spacer module between any of the antigen-binding module, the transmembrane module, and the co-stimulatory signaling module. In some embodiments, the spacer module comprises one or more peptide linkers connecting two CSR modules. In some embodiments, the spacer module comprises one or more peptide linkers between about 5 to about 70 (such as about any of 5, 10, 15, 20, 25, 30, 35, 40, 45, 50, 55, 60, 65, or 70, including any ranges between these values) amino acids in length.

In some embodiments, the target-binding module is an antibody moiety. In some embodiments, the antibody moiety is a Fab, a Fab′, a (Fab′)2, an Fv, or a single chain Fv (scFv). In some embodiments, the anti-WTMC antibody moiety comprises the CDRs or variables domains (V_{H} and/or V_{L} domains) of an antibody moiety that specifically binds to a target, such as any of antibody moieties described elsewhere herein.

The present disclosure also provides effector cells (such as T cells) that co-express a caTCR and a CSR or co-express a CAR and a CSR. Such effector cells are also referred to herein as “caTCR plus CSR effector cells” or “caTCR plus CSR effector cells”, respectively. In some embodiments, the caTCR plus CSR effector cell (such as a T cell) comprises a nucleic acid sequence encoding the CSR operably linked to an inducible promoter, including any of the inducible promoters described herein. In some embodiments, the expression of the CSR in the caTCR plus CSR effector cell (such as a T cell) is inducible upon signaling through the caTCR. In some such embodiments, the caTCR plus CSR effector cell (such as a T cell) comprises a nucleic acid sequence encoding the CSR operably linked to a promoter or regulatory element that is responsive to signaling through the caTCR. In some embodiments, the nucleic acid sequence encoding the CSR is operably linked to a nuclear-factor of the activated T-cell (NFAT)-derived promoter. In some embodiments, the NFAT-derived promoter is an NFAT-derived minimal promoter (see for example Durand, D. et. al., Molec. Cell. Biol. 8, 1715-1724 (1988); Clipstone, NA, Crabtree, GR. Nature. 1992 357(6380): 695-7; Chmielewski, M., et al. Cancer research 71.17 (2011): 5697-5706; and Zhang, L., et al. Molecular therapy 19.4 (2011): 751-759). Further description of CSRs may be found in U.S. Application No. 62/490,578, filed Apr. 26, 2017, and WO2018/200583, which are incorporated by reference herein in their entirety.

The present disclosure also provides effector cells (such as T cells) that express a caTCR or a CAR and anti-WTMC CSR (such as an anti-WTMC CSR described herein). Such effector cells are also referred to herein as “caTCR plus anti-WTMC CSR effector cells.” In some embodiments, the caTCR plus anti-WTMC CSR effector cell (such as a T cell) comprises a nucleic acid sequence encoding the anti-WTMC CSR operably linked to an inducible promoter, including any of the inducible promoters described herein. In some embodiments, the expression of the anti-WTMC CSR in the caTCR plus anti-WTMC CSR effector cell (such as a T cell) is inducible upon signaling through the caTCR. In some such embodiments, the caTCR plus anti-WTMC CSR effector cell (such as a T cell) comprises a nucleic acid sequence encoding the anti-WTMC CSR operably linked to a promoter or regulatory element that is responsive to signaling through the caTCR. In some embodiments, the nucleic acid sequence encoding the anti-WTMC CSR is operably linked to a nuclear-factor of the activated T-cell (NFAT)-derived promoter. In some embodiments, the caTCR expressed by the caTCR plus anti-WTMC CSR effector cell (such as a T cell) is an anti-WTMC caTCR. In some embodiments, the caTCR expressed by the caTCR plus anti-WTMC CSR effector cell (such as a T cell) is not an anti-WTMC caTCR and targets a different antigen.

In some embodiments, the CSR comprises a target-binding domain (e.g., an antibody moiety) that specifically binds a WTMC. In some embodiments, the CSR comprises a target-binding domain (e.g., an antibody moiety) that does not specifically bind a WTMC. In some embodiments, the CSR comprises two or more target-binding domains (e.g., antibody moieties) that specifically bind to the same or different targets. In some embodiments, the CSR is monospecific. In some embodiments, the CSR is multispecific, such as bispecific.

In some embodiments, the target-binding domain is an antibody moiety, such as a Fab, a Fab′, a (Fab′)2, an Fv, or a single chain Fv (scFv). In some embodiments, the target-binding domain is an extracellular domain of a receptor or a ligand. In some embodiments, the target-binding domain is monospecific. In some embodiments, the target-binding domain is multispecific, e.g., bispecific. In some embodiments, the target-binding domain specifically binds a cell surface protein. In some embodiments, the target-binding domain specifically binds a complex comprising a peptide and an MHC molecule.

In some embodiments, the target-binding domain specifically binds a target expressed or present on a cancer cell. In some embodiments, the target-binding domain specifically binds a target expressed or present on a leukemia cell. In some embodiments, the target-binding domain specifically binds CD33, CD371, CD123, and/or CD15. In some embodiments, the target-binding domain specifically binds a peptide/MHC complex, wherein the peptide is WT1. In some embodiments, the target-binding domain specifically binds a target expressed or present on a cancer type.

In some embodiments, the anti-WTMC CSR comprises a single polypeptide chain comprising: a) any one of the anti-WTMC antibody moieties described herein; b) a transmembrane module; and c) a co-stimulatory signaling module. In some embodiments, the anti-WTMC CSR comprises a single polypeptide chain comprising: a) an anti-WTMC antibody moiety comprising a HC-CDR1 comprising an amino acid sequence set forth in SEQ ID NO: 1; a HC-CDR2 comprising an amino acid sequence set forth in SEQ ID NO: 2; a HC-CDR3 comprising an amino acid sequence set forth in SEQ ID NO: 3; a LC-CDR1 comprising an amino acid sequence set forth in SEQ ID NO: 4; a LC-CDR2 comprising an amino acid sequence set forth in SEQ ID NO: 5; and a LC-CDR3 comprising an amino acid sequence set forth in SEQ ID NO: 6; b) a transmembrane module; and c) a co-stimulatory signaling module. In some embodiments, the CDRs are human CDRs. In some embodiments, the anti-WTMC CSR comprises a single polypeptide chain comprising: a) an anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 85 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 86; b) a transmembrane module; and c) a co-stimulatory signaling module. In some embodiments, the anti-WTMC CSR comprises a single polypeptide chain comprising: a) an anti-WTMC antibody moiety comprising a V_{H} domain comprising SEQ ID NO: 85 and a V_{L} domain comprising SEQ ID NO: 86 b) a transmembrane module; and c) a co-stimulatory signaling module.

In some embodiments, the anti-WTMC CSR comprises a single polypeptide chain comprising: a) an anti-WTMC antibody moiety comprising a HC-CDR1 comprising an amino acid sequence set forth in SEQ ID NO: 7; a HC-CDR2 comprising an amino acid sequence set forth in SEQ ID NO: 8; a HC-CDR3 comprising an amino acid sequence set forth in SEQ ID NO: 9; a LC-CDR1 comprising an amino acid sequence set forth in SEQ ID NO: 10; a LC-CDR2 comprising an amino acid sequence set forth in SEQ ID NO: 11; and a LC-CDR3 comprising an amino acid sequence set forth in SEQ ID NO: 12; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 87 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 88; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising SEQ ID NO: 87 and a V_{L} domain comprising SEQ ID NO: 88; b) a transmembrane module; and c) a co-stimulatory signaling module.

In some embodiments, the anti-WTMC CSR comprises a single polypeptide chain comprising: a) an anti-WTMC antibody moiety comprising a HC-CDR1 comprising an amino acid sequence set forth in SEQ ID NO: 13; a HC-CDR2 comprising an amino acid sequence set forth in SEQ ID NO: 14; a HC-CDR3 comprising an amino acid sequence set forth in SEQ ID NO: 15; a LC-CDR1 comprising an amino acid sequence set forth in SEQ ID NO: 16; a LC-CDR2 comprising an amino acid sequence set forth in SEQ ID NO: 17; and a LC-CDR3 comprising an amino acid sequence set forth in SEQ ID NO: 18; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 89 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 90; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising SEQ ID NO: 89 and a V_{L} domain comprising SEQ ID NO: 90; b) a transmembrane module; and c) a co-stimulatory signaling module.

In some embodiments, the anti-WTMC CSR comprises a single polypeptide chain comprising: a) an anti-WTMC antibody moiety comprising a HC-CDR1 comprising an amino acid sequence set forth in SEQ ID NO: 19; a HC-CDR2 comprising an amino acid sequence set forth in SEQ ID NO: 20; a HC-CDR3 comprising an amino acid sequence set forth in SEQ ID NO: 21; a LC-CDR1 comprising an amino acid sequence set forth in SEQ ID NO: 22; a LC-CDR2 comprising an amino acid sequence set forth in SEQ ID NO: 23; and a LC-CDR3 comprising an amino acid sequence set forth in SEQ ID NO: 24; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 91 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 92; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising SEQ ID NO: 91 and a V_{L} domain comprising SEQ ID NO: 92; b) a transmembrane module; and c) a co-stimulatory signaling module.

In some embodiments, the anti-WTMC CSR comprises a single polypeptide chain comprising: a) an anti-WTMC antibody moiety comprising a HC-CDR1 comprising an amino acid sequence set forth in SEQ ID NO: 25; a HC-CDR2 comprising an amino acid sequence set forth in SEQ ID NO: 26; a HC-CDR3 comprising an amino acid sequence set forth in SEQ ID NO: 27; a LC-CDR1 comprising an amino acid sequence set forth in SEQ ID NO: 28; a LC-CDR2 comprising an amino acid sequence set forth in SEQ ID NO: 29; and a LC-CDR3 comprising an amino acid sequence set forth in SEQ ID NO: 30; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 93 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 94; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising SEQ ID NO: 93 and a V_{L} domain comprising SEQ ID NO: 94; b) a transmembrane module; and c) a co-stimulatory signaling module.

In some embodiments, the anti-WTMC CSR comprises a single polypeptide chain comprising: a) an anti-WTMC antibody moiety comprising a HC-CDR1 comprising an amino acid sequence set forth in SEQ ID NO: 31; a HC-CDR2 comprising an amino acid sequence set forth in SEQ ID NO: 32; a HC-CDR3 comprising an amino acid sequence set forth in SEQ ID NO: 33; a LC-CDR1 comprising an amino acid sequence set forth in SEQ ID NO: 34; a LC-CDR2 comprising an amino acid sequence set forth in SEQ ID NO: 35; and a LC-CDR3 comprising an amino acid sequence set forth in SEQ ID NO: 36; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 95 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 96; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising SEQ ID NO: 95 and a V_{L} domain comprising SEQ ID NO: 96; b) a transmembrane module; and c) a co-stimulatory signaling module.

In some embodiments, the anti-WTMC CSR comprises a single polypeptide chain comprising: a) an anti-WTMC antibody moiety comprising a HC-CDR1 comprising an amino acid sequence set forth in SEQ ID NO: 37; a HC-CDR2 comprising an amino acid sequence set forth in SEQ ID NO: 38; a HC-CDR3 comprising an amino acid sequence set forth in SEQ ID NO: 39; a LC-CDR1 comprising an amino acid sequence set forth in SEQ ID NO: 40; a LC-CDR2 comprising an amino acid sequence set forth in SEQ ID NO: 41; and a LC-CDR3 comprising an amino acid sequence set forth in SEQ ID NO: 42; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 97 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 98; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising SEQ ID NO: 97 and a V_{L} domain comprising SEQ ID NO: 98; b) a transmembrane module; and c) a co-stimulatory signaling module.

In some embodiments, the anti-WTMC CSR comprises a single polypeptide chain comprising: a) an anti-WTMC antibody moiety comprising a HC-CDR1 comprising an amino acid sequence set forth in SEQ ID NO: 43; a HC-CDR2 comprising an amino acid sequence set forth in SEQ ID NO: 44; a HC-CDR3 comprising an amino acid sequence set forth in SEQ ID NO: 45; a LC-CDR1 comprising an amino acid sequence set forth in SEQ ID NO: 46; a LC-CDR2 comprising an amino acid sequence set forth in SEQ ID NO: 47; and a LC-CDR3 comprising an amino acid sequence set forth in SEQ ID NO: 48; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 99 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 100; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising SEQ ID NO: 99 and a V_{L} domain comprising SEQ ID NO: 100; b) a transmembrane module; and c) a co-stimulatory signaling module.

In some embodiments, the anti-WTMC CSR comprises a single polypeptide chain comprising: a) an anti-WTMC antibody moiety comprising a HC-CDR1 comprising an amino acid sequence set forth in SEQ ID NO: 49; a HC-CDR2 comprising an amino acid sequence set forth in SEQ ID NO: 50; a HC-CDR3 comprising an amino acid sequence set forth in SEQ ID NO: 51; a LC-CDR1 comprising an amino acid sequence set forth in SEQ ID NO: 52; a LC-CDR2 comprising an amino acid sequence set forth in SEQ ID NO: 53; and a LC-CDR3 comprising an amino acid sequence set forth in SEQ ID NO: 54; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 101 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 102; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising SEQ ID NO: 101 and a V_{L} domain comprising SEQ ID NO: 102; b) a transmembrane module; and c) a co-stimulatory signaling module.

In some embodiments, the anti-WTMC CSR comprises a single polypeptide chain comprising: a) an anti-WTMC antibody moiety comprising a HC-CDR1 comprising an amino acid sequence set forth in SEQ ID NO: 55; a HC-CDR2 comprising an amino acid sequence set forth in SEQ ID NO: 56; a HC-CDR3 comprising an amino acid sequence set forth in SEQ ID NO: 57; a LC-CDR1 comprising an amino acid sequence set forth in SEQ ID NO: 58; a LC-CDR2 comprising an amino acid sequence set forth in SEQ ID NO: 59; and a LC-CDR3 comprising an amino acid sequence set forth in SEQ ID NO: 60; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 103 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 104; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising SEQ ID NO: 103 and a V_{L} domain comprising SEQ ID NO: 104; b) a transmembrane module; and c) a co-stimulatory signaling module.

In some embodiments, the anti-WTMC CSR comprises a single polypeptide chain comprising: a) an anti-WTMC antibody moiety comprising a HC-CDR1 comprising an amino acid sequence set forth in SEQ ID NO: 61; a HC-CDR2 comprising an amino acid sequence set forth in SEQ ID NO: 62; a HC-CDR3 comprising an amino acid sequence set forth in SEQ ID NO: 63; a LC-CDR1 comprising an amino acid sequence set forth in SEQ ID NO: 64; a LC-CDR2 comprising an amino acid sequence set forth in SEQ ID NO: 65; and a LC-CDR3 comprising an amino acid sequence set forth in SEQ ID NO: 66; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 105 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 106; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising SEQ ID NO: 105 and a V_{L} domain comprising SEQ ID NO: 106; b) a transmembrane module; and c) a co-stimulatory signaling module.

In some embodiments, the anti-WTMC CSR comprises a single polypeptide chain comprising: a) an anti-WTMC antibody moiety comprising a HC-CDR1 comprising an amino acid sequence set forth in SEQ ID NO: 67; a HC-CDR2 comprising an amino acid sequence set forth in SEQ ID NO: 68; a HC-CDR3 comprising an amino acid sequence set forth in SEQ ID NO: 69; a LC-CDR1 comprising an amino acid sequence set forth in SEQ ID NO: 70; a LC-CDR2 comprising an amino acid sequence set forth in SEQ ID NO: 71; and a LC-CDR3 comprising an amino acid sequence set forth in SEQ ID NO: 72; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 107 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 108; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising SEQ ID NO: 107 and a V_{L} domain comprising SEQ ID NO: 108; b) a transmembrane module; and c) a co-stimulatory signaling module.

In some embodiments, the anti-WTMC CSR comprises a single polypeptide chain comprising: a) an anti-WTMC antibody moiety comprising a HC-CDR1 comprising an amino acid sequence set forth in SEQ ID NO: 73; a HC-CDR2 comprising an amino acid sequence set forth in SEQ ID NO: 74; a HC-CDR3 comprising an amino acid sequence set forth in SEQ ID NO: 75; a LC-CDR1 comprising an amino acid sequence set forth in SEQ ID NO: 76; a LC-CDR2 comprising an amino acid sequence set forth in SEQ ID NO: 77; and a LC-CDR3 comprising an amino acid sequence set forth in SEQ ID NO: 78; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 109 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 110; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising SEQ ID NO: 109 and a V_{L} domain comprising SEQ ID NO: 110; b) a transmembrane module; and c) a co-stimulatory signaling module.

In some embodiments, the anti-WTMC CSR comprises a single polypeptide chain comprising: a) an anti-WTMC antibody moiety comprising a HC-CDR1 comprising an amino acid sequence set forth in SEQ ID NO: 79; a HC-CDR2 comprising an amino acid sequence set forth in SEQ ID NO: 80; a HC-CDR3 comprising an amino acid sequence set forth in SEQ ID NO: 81; a LC-CDR1 comprising an amino acid sequence set forth in SEQ ID NO: 82; a LC-CDR2 comprising an amino acid sequence set forth in SEQ ID NO: 83; and a LC-CDR3 comprising an amino acid sequence set forth in SEQ ID NO: 84; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 111 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 112; b) a transmembrane module; and c) a co-stimulatory signaling module. In some embodiments, the anti-WTMC CSR comprises a single polypeptide chain comprising: a) anti-WTMC antibody moiety comprising a V_{H} domain comprising SEQ ID NO: 111 and a V_{L} domain comprising SEQ ID NO: 112; b) a transmembrane module; and c) a co-stimulatory signaling module.

In some embodiments, the anti-WTMC CSR comprises a single polypeptide chain comprising: a) the anti-WTMC antibody moiety of Clone 1, 2, 10, 11, 12, 14, 15, 17, 18, 26, 30, 32, 34, or 36; b) a transmembrane module; and c) a co-stimulatory signaling module. In some embodiments, the anti-WTMC CSR comprises the anti-WTMC antibody moiety of Clone 18 or Clone 34.

In some embodiments, the CSR comprises a) an anti-CD33 antibody moiety comprising a HC-CDR1 comprising an amino acid sequence set forth in SEQ ID NO: 319, a HC-CDR2 comprising an amino acid sequence set forth in SEQ ID NO: 320, a HC-CDR3 comprising an amino acid sequence set forth in SEQ ID NO: 321, a LC-CDR1 comprising an amino acid sequence set forth in SEQ ID NO: 322, a LC-CDR2 comprising an amino acid sequence set forth in SEQ ID NO: 323, and a LC-CDR3 comprising an amino acid sequence set forth in SEQ ID NO: 324; b) a transmembrane module; and c) a co-stimulatory signaling module.

In some embodiments, the anti-CD33 CSR comprises the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to the V_{H} domain comprising the amino acid sequence of SEQ ID NO: 317 and/or the V_{L} domain comprising the amino acid sequence of SEQ ID NO: 318. In some embodiments, the anti-CD33 CSR comprises a V_{H} domain comprising the amino acid sequence of SEQ ID NO: 317 and a V_{L} domain comprising the amino acid sequence of SEQ ID NO: 318.

In any of the preceding embodiments, the co-stimulatory signaling module comprises an intracellular signaling domain of CD28. In some embodiments, the intracellular signaling domain comprises the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 283. In some embodiments, the intracellular signaling domain comprises the amino acid sequence of SEQ ID NO: 283.

In any of the foregoing embodiments, the intracellular domain comprises a CD30 costimulatory intracellular signaling sequence. In some embodiments, the intracellular signaling domain comprises the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 284. In some embodiments, the intracellular signaling domain comprises the amino acid sequence of SEQ ID NO: 284. In some embodiments, the co-stimulatory signaling module comprises an intracellular signaling domain of 4-1BB. In some embodiments, the co-stimulatory signaling module comprises an intracellular signaling domain of ICOS.

### Construct Combinations

In some aspects, provided herein are construct combinations that comprise at least two different constructs described herein. In some embodiments, at least one of the constructs is an anti-WTMC construct described herein, comprising an anti-WTMC antibody moiety (such as any one of the anti-WTMC antibody moieties described in the “Anti-WTMC Antibody Moieties” section). In some embodiments, the at least two different constructs are the same format, e.g., at least two different antibodies (e.g., two different full-length IgG antibodies or two different bispecific antibodies), at least two different CARs, or at least two different caTCRs. In some embodiments, the at least two different constructs are different formats, e.g., an antibody and a CAR; an antibody and a caTCR; a CAR and a CSR; a caTCR and a CSR, etc.

Also provided are construct combinations that comprise at least two different anti-WTMC constructs described herein, comprising an anti-WTMC antibody moiety (such as any one of the anti-WTMC antibody moieties described in the “Anti-WTMC Antibody Moieties” section). In some embodiments, the at least two different anti-WTMC constructs are the same format, e.g., at least two different antibodies (e.g., two different full-length IgG antibodies or two different bispecific antibodies), at least two different CARs, at least two different caTCRs, or at least two different CSRs. In some embodiments, the at least two different anti-WTMC constructs are different formats, e.g., an antibody and a CAR; an antibody and a caTCR; a CAR and a CSR; a caTCR and a CSR, etc.

In some embodiments, the caTCR, CAR or TCR and the CSR of an anti-WTMC construct combination provided herein are encoded on separate nucleic acids. In some embodiments, the separate nucleic acids are each expressed (e.g., separately) and translated (e.g., separately) in a cell (such as an anti-WTMC effector cell, which is described in further detail elsewhere herein). In some embodiments, the caTCR, CAR or TCR and the CSR of an anti-WTMC construct combination provided herein are encoded on the same nucleic acid (e.g., a single nucleic acid). In some embodiments, the single nucleic acid encoding the caTCR, CAR or TCR and CSR construct combination is expressed and translated to generate a single polypeptide which is subsequently processed (e.g., such as cleaved during or following translation) into separate polypeptides, e.g., the caTCR, CAR or TCR polypeptide(s) and CSR polypeptide.

In some embodiments, a single nucleic acid encoding a caTCR, CAR or TCR and a CSR construct combination expresses a polypeptide comprising (from N-terminus to C-terminus) the amino acid sequence(s) of a caTCR, CAR or TCR construct, a peptide linker, and the amino acid sequence of a CSR construct. In some embodiments, a single nucleic acid encoding a caTCR, CAR or TCR and a CSR construct combination expresses a polypeptide comprising (from N-terminus to C-terminus) the amino acid sequence of a CSR construct, a peptide linker, and the amino acid sequence(s) of a caTCR, CAR or TCR construct. In some embodiments, the nucleic acid further encodes, e.g., one or more peptide linkers, peptide spacers, peptide tags, signal peptides and/or other amino acid sequences (see, e.g., Tables F-1 and F-2 for exemplary linker sequences and tag sequences).

In some embodiments, the anti-WTMC construct combination comprises a CSR, CAR, or caTCR construct that binds to a target other than a WTMC complex. In some embodiments, the anti-WTMC construct combination comprises a CSR that binds to a target other than a WTMC complex, wherein the CSR comprises a target-binding domain selected from the group consisting of: (i) a CD33-binding module (e.g., an anti-CD33 antibody moiety), (ii) a CD371, CD123, or CD15-binding module (e.g., an anti-CD371, anti-CD123, or anti-CD15 antibody moiety), (iii) a CD33- and a CD371-, CD123-, or CD15-binding module (e.g., a bispecific antibody moiety that specifically binds CD33 and any one of CD371, CD123, or CD15), (iv) a MUC16 binding module (e.g. an anti-MUC16 antibody moiety), and (v) a HER2-binding module (e.g., an anti-HER2 antibody moiety).

In some embodiments, the anti-WTMC construct combination comprises a CAR that binds to a target other than a WTMC complex, wherein the CAR comprises a target-binding domain selected from the group consisting of: (i) a CD33-binding module (e.g., an anti-CD33 antibody moiety), (ii) a CD371, CD123, or CD15-binding module (e.g., an anti-CD371, anti-CD123, or anti-CD15 antibody moiety), (iii) a CD33- and a CD371, CD123, or CD15-binding module (e.g., a bispecific antibody moiety that specifically binds CD33 and any one of CD371, CD123, or CD15), (iv) a MUC16-binding module (e.g., an anti-MUC16 antibody moiety), and (v) a HER2-binding module (e.g., an anti-HER2 antibody moiety).

In some embodiments, the anti-WTMC construct combination comprises a caTCR that binds to a target ligand other than a WTMC complex, wherein the caTCR comprises a target-binding domain selected from the group consisting of (i) a CD33-binding module (e.g., an anti-CD33 antibody moiety), (ii) a CD371, CD123, or CD15-binding module (e.g., an anti-CD371, anti-CD123, or anti-CD15 antibody moiety), (iii) a CD33- and a CD371, CD123, or CD15-binding module (e.g., a bispecific antibody moiety that specifically binds CD33 and any one of CD371, CD123, or CD15), (iv) a MUC16-binding module (e.g., an anti-MUC16 antibody moiety), and (v) a HER2-binding module (e.g., an anti-HER2 antibody moiety).

In some embodiments, the non-WTMC target bound by the CSR, CAR, or caTCR construct is expressed on a cancer cell. In some embodiments, the non-WTMC target bound by the CSR, CAR, or caTCR construct is expressed on a WT1-positive cancer cell (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma).

In some embodiments, a CD33-binding module (e.g., an anti-CD33 antibody moiety) specifically binds CD33. In some embodiments, the anti-CD33 antibody moiety comprises a HC-CDR1 comprising an amino acid sequence set forth in SEQ ID NO: 319, a HC-CDR2 comprising an amino acid sequence set forth in SEQ ID NO: 320, a HC-CDR3 comprising an amino acid sequence set forth in SEQ ID NO: 321, a LC-CDR1 comprising an amino acid sequence set forth in SEQ ID NO: 322, a LC-CDR2 comprising an amino acid sequence set forth in SEQ ID NO: 323, and a LC-CDR3 comprising an amino acid sequence set forth in SEQ ID NO: 324. In some embodiments, the anti-CD33 antibody moiety comprises a heavy chain variable domain (V_{H}) comprising the amino acid sequence of SEQ ID NO: 317, or a variant thereof having at least about 85% (e.g., at least about any one of 85%, 86%, 87%, 88%, 89%, 90%, 91%, 92%, 93%, 94%, 95%, 96%, 97%, 98%, 99% or 100%) sequence identity, and a light chain variable domain (V_{L}) comprising the amino acid sequence of SEQ ID NO: 318, or a variant thereof having at least about 85% (e.g., at least about any one of 85%, 86%, 87%, 88%, 89%, 90%, 91%, 92%, 93%, 94%, 95%, 96%, 97%, 98%, 99% or 100%) sequence identity. In some embodiments, the anti-CD33 antibody moiety comprises a V_{H} comprising the amino acid sequence of SEQ ID NO: 317 and a V_{L} comprising the amino acid sequence of SEQ ID NO: 318. Additional anti-CD33 antibody, including scFv, sequences can be found in the art. In some embodiments, the anti-CD33 CSR is a bispecific anti-CD33 CSR.

In some embodiments, a CD371, CD123, or CD15-binding module (e.g., an anti-CD371, anti-CD123, or anti-CD15 antibody moiety) specifically binds CD371, CD123, or CD15. The binding module that specifically binds to CD371, CD123, or CD15 may comprise any anti-CD371, anti-CD123, or anti-CD15 antibody moieties comprising the CDR, V_{H}, and/or V_{L} sequences as described in the art.

In some embodiments, a MUC16-binding module (e.g., an anti-MUC16 antibody moiety) specifically binds MUC16. The binding module that specifically binds to MUC16 may comprise any anti-MUC16 antibody moieties comprising the CDR, V_{H}, and/or V_{L} sequences as described in the art (WO2011119979, WO2020/102555, WO2020/227538, and WO2016/149368, which are incorporated by reference herein).

In some embodiments, a HER2-binding module (e.g., an anti-HER2 antibody moiety) specifically binds HER2. The binding module that specifically binds to HER2 may comprise any anti-HER2 antibody moieties comprising the CDR, V_{H}, and/or V_{L} sequences as described in the art (e.g., trastuzumab).

In some embodiments, a WT1 peptide (e.g., WT1-RMF) and a MHC class I protein (WT1/MHC)-binding module (e.g., an anti-WT1/MHC antibody moiety) specifically binds to a WTMC. In some embodiments, the anti-WT1/MHC antibody moiety comprises (a) a HC-CDR1 comprising an amino acid sequence set forth in SEQ ID NO: 1; (b) a HC-CDR2 comprising an amino acid sequence set forth in SEQ ID NO: 2; (c) a HC-CDR3 comprising an amino acid sequence set forth in SEQ ID NO: 3; (d) a LC-CDR1 comprising an amino acid sequence set forth in SEQ ID NO: 4; (e) a LC-CDR2 comprising an amino acid sequence set forth in SEQ ID NO: 5; and (f) a LC-CDR3 comprising an amino acid sequence set forth in SEQ ID NO: 6. In some embodiments, the CDRs are human CDRs. In some embodiments, the anti-WT1/MHC antibody moiety comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 85 and/or a V_{L}. domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 86. In some embodiments, the anti-WT1/MHC antibody moiety comprises a V_{H} domain comprising SEQ ID NO: 85 and a V_{L}. domain comprising SEQ ID NO: 86.

In some embodiments, the anti-WT1/MHC antibody moiety comprises (a) a IC-CDR1 comprising an amino acid sequence set forth in SEQ ID NO: 7; (b) a HC-CDR2 comprising an amino acid sequence set forth in SEQ ID NO: 8; (c) a HC-CDR3 comprising an amino acid sequence set forth in SEQ ID NO: 9; (d) a LC-CDR1 comprising an amino acid sequence set forth in SEQ ID NO: 10; (e) a LC-CDR2 comprising an amino acid sequence set forth in SEQ ID NO: 11; and (f) a LC-CDR3 comprising an amino acid sequence set forth in SEQ ID NO: 12. In some embodiments, the CDRs are human CDRs. In some embodiments, the anti-WT1/MHC antibody moiety comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 87 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 88. In some embodiments, the anti-WT1/MHC antibody moiety comprises a V_{H} domain comprising SEQ ID NO: 87 and a V_{L} domain comprising SEQ ID NO: 88.

In some embodiments, the anti-WT1/MHC antibody moiety comprises: (a) a HC-CDR1 comprising an amino acid sequence set forth in SEQ ID NO: 13; (b) a HC-CDR2 comprising an amino acid sequence set forth in SEQ ID NO: 14; (c) a HC-CDR3 comprising an amino acid sequence set forth in SEQ ID NO: 15; (d) a LC-CDR1 comprising an amino acid sequence set forth in SEQ ID NO: 16; (e) a LC-CDR2 comprising an amino acid sequence set forth in SEQ ID NO: 17; and (f) a LC-CDR3 comprising an amino acid sequence set forth in SEQ ID NO: 18. In some embodiments, the CDRs are human CDRs. In some embodiments, the WT1/MHC antibody moiety comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 89 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 90. In some embodiments, the anti-WT1/MHC antibody moiety comprises a V_{H} domain comprising SEQ ID NO: 89 and a V_{L} domain comprising SEQ ID NO: 90.

In some embodiments, the anti-WT1/MHC antibody moiety comprises (a) a HC-CDR1 comprising an amino acid sequence set forth in SEQ ID NO: 19; (b) a HC-CDR2 comprising an amino acid sequence set forth in SEQ ID NO: 20; (c) a HC-CDR3 comprising an amino acid sequence set forth in SEQ ID NO: 21; (d) a LC-CDR1 comprising an amino acid sequence set forth in SEQ ID NO: 22; (e) a LC-CDR2 comprising an amino acid sequence set forth in SEQ ID NO: 23; and (f) a LC-CDR3 comprising an amino acid sequence set forth in SEQ ID NO: 24. In some embodiments, the CDRs are human CDRs. In some embodiments, the anti-WT1/MHC antibody moiety comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 91 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 92. In some embodiments, the anti-WT1/MIC antibody moiety comprises a V_{H} domain comprising SEQ ID NO: 91 and a V_{L} domain comprising SEQ ID NO: 92.

In some embodiments, the anti-WT1/H1-IC antibody moiety comprises (a) a HC-CDR1 comprising an amino acid sequence set forth in SEQ ID NO: 25; (b) a HC-CDR2 comprising an amino acid sequence set forth in SEQ ID NO: 26; (c) a HC-CDR3 comprising an amino acid sequence set forth in SEQ ID NO: 27; (d) a LC-CDR1 comprising an amino acid sequence set forth in SEQ ID NO: 28; (e) a LC-CDR2 comprising an amino acid sequence set forth in SEQ ID NO: 29; and (1) a LC-CDR3 comprising an amino acid sequence set forth in SEQ ID NO: 30. In some embodiments, the CDRs are human CDRs. In some embodiments, the anti-WT1/MHC antibody moiety comprises a V_{H}1 domain comprising the amino acid sequence that is at least about 85% (e.g., at least about any one of 85% 86%, 87%, 88% 89%, 90%, 91%, 92%, 93%, 94% 95% 96%, 97%, 98%, 99% or 100%) identical to SEQ ID NO: 93 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 94. In some embodiments, the anti-WT1/MHC antibody moiety comprises a V_{H} domain comprising SEQ ID NO: 93 and a V_{L} domain comprising SEQ ID NO: 94.

In some embodiments, the anti-WT1/MHC antibody moiety comprises (a) a HC-CDR1 comprising an amino acid sequence set forth in SEQ ID NO: 31; (b) a HC-CDR2 comprising an amino acid sequence set forth in SEQ ID NO: 32; (c) a HC-CDR3 comprising an amino acid sequence set forth in SEQ ID NO: 33; (d) a LC-CDR1 comprising an amino acid sequence set forth in SEQ ID NO: 34; (e) a LC-CDR2 comprising an amino acid sequence set forth in SEQ ID NO: 35; and (f) a LC-CDR3 comprising an amino acid sequence set forth in SEQ ID NO: 36. In some embodiments, the CDRs are human CDRs. In some embodiments, the anti-WT1/MHC antibody moiety comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 95 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 96. In some embodiments, the anti-WT1/MHC antibody moiety comprises a V_{H} domain comprising SEQ ID NO: 95 and a V_{L} domain comprising SEQ ID NO: 96.

In some embodiments, the anti-WT1/MHC antibody moiety comprises (a) a HC-CDR1 comprising an amino acid sequence set forth in SEQ ID NO: 37; (b) a HC-CDR2 comprising an amino acid sequence set forth in SEQ ID NO: 38; (c) a HC-CDR3 comprising an amino acid sequence set forth in SEQ ID NO: 39; (d) a LC-CDR1 comprising an amino acid sequence set forth in SEQ ID NO: 40; (e) a LC-CDR2 comprising an amino acid sequence set forth in SEQ ID NO: 41; and (f) a LC-CDR3 comprising an amino acid sequence set forth in SEQ ID NO: 42. In some embodiments, the CDRs are human CDRs. In some embodiments, the anti-WT1/MHC antibody moiety comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 97 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 98. In some embodiments, the anti-WT1/MHC antibody moiety comprises a V_{H} domain comprising SEQ ID NO: 97 and a V_{L} domain comprising SEQ ID NO: 98.

In some embodiments, the anti-WT1/MHC antibody moiety comprises (a) a HC-CDR1 comprising an amino acid sequence set forth in SEQ ID NO: 43; (b) a HC-CDR2 comprising an amino acid sequence set forth in SEQ ID NO: 44; (c) a HC-CDR3 comprising an amino acid sequence set forth in SEQ ID NO: 45; (d) a LC-CDR I comprising an amino acid sequence set forth in SEQ ID NO: 46; (e) a LC-CDR2 comprising an amino acid sequence set forth in SEQ ID NO: 47; and (f) a LC-CDR3 comprising an amino acid sequence set forth in SEQ ID NO: 48. In some embodiments, the CDRs are human CDRs. In some embodiments, the anti-WT1/MHC antibody moiety comprises a V_{H} domain comprising the amino acid sequence that is at least about 85% (e.g., at least about any one of 85%, 86%, 87%, 88%, 89%, 90%, 91%, 92%, 93%, 94%, 95%, 96% 97%, 98%, 99% or 100%) identical to SEQ ID NO: 99 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 100. In some embodiments, the anti-WT1/MHC antibody moiety comprises a V_{H} domain comprising SEQ ID NO: 99 and a V_{L} domain comprising SEQ ID NO: 100.

In some embodiments, the anti-WT1/MIC antibody moiety comprises (a) a IC-CDR1 comprising an amino acid sequence set forth in SEQ ID NO: 49; (b) a HC-CDR2 comprising an amino acid sequence set forth in SEQ ID NO: 50; (c) a HC-CDR3 comprising an amino acid sequence set forth in SEQ ID NO: 51; (d) a LC-CDR1 comprising an amino acid sequence set forth in SEQ ID NO: 52; (e) a LC-CDR2 comprising an amino acid sequence set forth in SEQ ID NO: 53; and (f) a LC-CDR3 comprising an amino acid sequence set forth in SEQ ID NO: 54. In some embodiments, the CDRs are human CDRs. In some embodiments, the anti-WT1/MHC antibody moiety comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 101 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 102. In some embodiments, the anti-WT1/MHC antibody moiety comprises a V_{H} domain comprising SEQ ID NO: 101 and a V_{L} domain comprising SEQ ID NO: 102.

In some embodiments, the anti-WT1/MHC antibody moiety comprises (a) a HC-CDR1 comprising an amino acid sequence set forth in SEQ ID NO: 55; (b) a HC-CDR2 comprising an amino acid sequence set forth in SEQ ID NO: 56; (c) a HC-CDR3 comprising an amino acid sequence set forth in SEQ ID NO: 57; (d) a LC-CDR1 comprising an amino acid sequence set forth in SEQ ID NO: 58; (e) a LC-CDR2 comprising an amino acid sequence set forth in SEQ ID NO: 59; and (f) a LC-CDR3 comprising an amino acid sequence set forth in SEQ ID NO: 60. In some embodiments, the CDRs are human CDRs. In some embodiments, the anti-WT1/MHC antibody moiety comprises a V domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 103 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 104. In some embodiments, the anti-WT1/MHC antibody moiety comprises a V_{H} domain comprising SEQ ID NO: 103 and a V_{L} domain comprising SEQ ID NO: 104.

In some embodiments, the anti-WT1/MHC antibody moiety comprises (a) a HC-CDR1 comprising an amino acid sequence set forth in SEQ ID NO: 61; (b) a HC-CDR2 comprising an amino acid sequence set forth in SEQ ID NO: 62; (c) a HC-CDR3 comprising an amino acid sequence set forth in SEQ ID NO: 63; (d) a LC-CDR1 comprising an amino acid sequence set forth in SEQ ID NO: 64; (e) a LC-CDR2 comprising an amino acid sequence set forth in SEQ ID NO: 65; and (f) a LC-CDR3 comprising an amino acid sequence set forth in SEQ ID NO: 66. In some embodiments, the CDRs are human CDRs. In some embodiments, the anti-WT1/MHC antibody moiety comprises a V_{H} domain comprising the amino acid sequence that is at least about 85% (e.g., at least about any one of 85% 86%, 87%, 88% 89%, 90%, 91%, 92%, 93%, 94% 95% 96% 97% 98%, 99% or 100%) identical to SEQ ID NO: 105 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 106. In some embodiments, the anti-WT1/MUC antibody moiety comprises a V_{H} domain comprising SEQ ID NO: 105 and a V_{L} domain comprising SEQ ID NO: 106.

In some embodiments, the anti-WT1/MHC antibody moiety comprises (a) a HC-CDR1 comprising an amino acid sequence set forth in SEQ ID NO: 67; (b) a HC-CDR2 comprising an amino acid sequence set forth in SEQ ID NO: 68; (c) a HC-CDR3 comprising an amino acid sequence set forth in SEQ ID NO: 69; (d) a LC-CDR1 comprising an amino acid sequence set forth in SEQ ID NO: 70; (e) a LC-CDR2 comprising an amino acid sequence set forth in SEQ ID NO: 71; and (f) a LC-CDR3 comprising an amino acid sequence set forth in SEQ ID NO: 72. In some embodiments, the CDRs are human CDRs. In some embodiments, the anti-WT1/MHC antibody moiety comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 107 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 108. In some embodiments, the anti-WT1/MHC antibody moiety comprises a V_{H} domain comprising SEQ ID NO: 107 and a V_{L} domain comprising SEQ ID NO: 108.

In some embodiments, the anti-WT1/MHC antibody moiety comprises (a) a HC-CDR1 comprising an amino acid sequence set forth in SEQ ID NO: 73; (b) a HC-CDR2 comprising an amino acid sequence set forth in SEQ ID NO: 74; (c) a HC-CDR3 comprising an amino acid sequence set forth in SEQ ID NO: 75; (d) a LC-CDR1 comprising an amino acid sequence set forth in SEQ ID NO: 76; (e) a LC-CDR2 comprising an amino acid sequence set forth in SEQ ID NO: 77; and (f) a LC-CDR3 comprising an amino acid sequence set forth in SEQ ID NO: 78. In some embodiments, the CDRs are human CDRs. In some embodiments, anti-WT1/MHC antibody moiety comprises a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 109 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 110. In some embodiments, the anti-WT1/MHC antibody moiety comprises a V_{H} domain comprising SEQ ID NO: 109 and a V_{L} domain comprising SEQ ID NO: 110.

In some embodiments, the anti-WT1/MHC antibody moiety comprises (a) a HC-CDR1 comprising an amino acid sequence set forth in SEQ ID NO: 79; (b) a HC-CDR2 comprising an amino acid sequence set forth in SEQ ID NO: 80; (c) a HC-CDR3 comprising an amino acid sequence set forth in SEQ ID NO: 81; (d) a LC-CDR1 comprising an amino acid sequence set forth in SEQ ID NO: 82; (e) a LC-CDR2 comprising an amino acid sequence set forth in SEQ ID NO: 83; and (f) a LC-CDR3 comprising an amino acid sequence set forth in SEQ ID NO: 84. In some embodiments, the CDRs are human CDRs. In some embodiments, the anti-WT1/MHC antibody moiety comprises a V_{H} domain comprising the amino acid sequence that is at least about 85% (e.g., at least about any one of 85% 86% 87%, 88%, 89%, 90% 91%, 92%, 93%, 94%, 95%, 96%, 97%, 98%, 99% or 100%) identical to SEQ ID NO: 111 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to SEQ ID NO: 112. In some embodiments, the anti-WT1/MHC antibody moiety comprises a V_{H} domain comprising SEQ ID NO: 111 and a V_{L} domain comprising SEQ ID NO: 112.

In some embodiments, the anti-WTMC construct combination provided herein is a CAR and CSR combination construct. In some embodiments, the CAR is an anti-WTMC CAR described herein, comprising an anti-WTMC antibody moiety (such as any one of the anti-WTMC antibody moieties described in the “Anti-WTMC Antibody Moieties” section) that binds to a complex comprising a WT1 peptide (e.g., WT1-RMF) and a MHC class I protein (e.g., a WTMC complex). In some embodiments, the CSR is an anti-WTMC CSR (i.e., a CSR that comprises a WTMC-binding module), e.g., such as described herein. In some embodiments, the anti-WTMC CSR described herein targets a complex comprising a WT1 peptide (e.g., WT1-RMF) peptide and a MHC class I protein (e.g., a WTMC complex). In some embodiments, the WT1 peptide (e.g., WT1-RMF) peptide comprises the amino acid sequence of SEQ ID NO: 113. In some embodiments, the anti-WTMC CSR specifically recognizes amino acid residues FPNA (SEQ ID NO: 349) of SEQ ID NO: 113. In some embodiments, the MHC class I protein is HLA-A*02:01. In some embodiments, the CSR specifically binds a WTMC that is different from the WTMC complex bound by the anti-WTMC CAR. In some embodiments, the CSR specifically binds a WTMC complex that is identical to the WTMC bound by the anti-WTMC CAR. In some embodiments, the CSR binds to a target ligand other than a WTMC complex.

In some embodiments, the anti-WTMC construct combination comprises an anti-WTMC CAR and a CSR, wherein the anti-WTMC CAR specifically binds a WTMC complex and the CSR specifically binds a target ligand that is not a WTMC. In some embodiments, the CSR comprises a CD33-binding module (e.g., any one of the anti-CD33 antibody moieties described herein). In some embodiments, the CSR (e.g., the anti-CD33 antibody moiety of CD33-binding module of the CSR) specifically binds CD33. In some embodiments, the anti-CD33 CSR comprises a single polypeptide chain comprising: a) an extracellular domain comprising any one of the anti-CD33 antibody moieties comprising the CDR, V_{H}, and/or V_{L} sequences as described in the present application; b) a transmembrane domain; and c) an intracellular signaling domain.

In some embodiments, the anti-WTMC construct combination comprises an anti-WTMC CAR and a CSR, wherein the anti-WTMC CAR specifically binds a WTMC complex and the CSR specifically binds CD371, CD123, or CD15. In some embodiments, the CSR comprises a CD371-, CD123-, or CD15-binding module (e.g., an anti-CD371, anti-CD123, or anti-CD15 antibody moiety). In some embodiments, the CSR (e.g., the anti-CD371, anti-CD123, or anti-CD15 antibody moiety of CD371-, CD123-, or CD15-binding module of the CSR) specifically binds CD371, CD123, or CD15. In some embodiments, the anti-CD371, anti-CD123, or anti-CD15 CSR comprises a single polypeptide chain comprising: a) an anti-CD371, anti-CD123, or anti-CD15 antibody moiety comprising any of the anti-CD371, anti-CD123, or anti-CD15 antibody moieties comprising the CDR, V_{H}, and/or V_{L} sequences as described in the present application; b) a transmembrane domain, and c) an intracellular signaling domain.

In some embodiments, the anti-WTMC construct combination comprises an anti-WTMC CAR and a CSR, wherein the anti-WTMC CAR specifically binds a WTMC complex and the CSR specifically binds a target that is not a WTMC, wherein the CSR is a multispecific CSR. In some embodiments, the CSR comprises a CD33-binding module (e.g., any of the anti-CD33 antibody moieties comprising the CDR, V_{H}, and/or V_{L} sequences as described in the present application) and further comprises a CD371, CD123, or CD15 binding module (e.g., any of the anti-CD371, anti-CD123, or anti-CD15 antibody moieties comprising the CDR, V_{H}, and/or V_{L} sequences as described in the art). In some embodiments, the multispecific CSR is a bispecific CSR, and specifically binds both CD33 and any one of CD371, CD123, or CD15.

In some embodiments, the anti-WTMC construct combination comprises an anti-WTMC CAR and a CSR, wherein the anti-WTMC CAR specifically binds a WTMC complex and the CSR specifically binds MUC16. In some embodiments, the CSR comprises a MUC16 binding module (e.g., an anti-MUC16 antibody moiety). In some embodiments, the CSR (e.g., the anti-MUC16 antibody moiety of MUC16 binding module of the CSR) specifically binds MUC16. In some embodiments, the anti-MUC16 CSR comprises a single polypeptide chain comprising: a) an anti-MUC16 antibody moiety comprising any of the anti-MUC16 antibody moieties comprising the CDR, V_{H}, and/or V_{L} sequences as described in the art; b) a transmembrane module; and c) and co-stimulatory signaling module

In some embodiments, the anti-WTMC construct combination comprises an anti-WTMC CAR and a CSR, wherein the anti-WTMC CAR specifically binds a WTMC complex and the CSR specifically binds HER2. In some embodiments, the CSR comprises a HER2-binding module (e.g., an anti-HER2 antibody moiety). In some embodiments, the CSR (e.g., the anti-HER2 antibody moiety of HER2-binding module of the CSR) specifically binds HER2. In some embodiments, the anti-HER2 CSR comprises a single polypeptide chain comprising: a) an anti-HER2 antibody moiety comprising any of the anti-HER2 antibody moieties comprising the CDR, V_{H}, and/or V_{L} sequences as described in the art; b) a transmembrane module; and c) and co-stimulatory signaling module.

In some embodiments, the anti-WTMC construct combination provided herein is a caTCR, and CSR combination construct. In some embodiments, the caTCR is an anti-WTMC caTCR i.e., a caTCR that comprises a WTMC-binding module), e.g., such as described herein. In some embodiments, the anti-WTMC caTCR comprises an anti-WTMC antibody moiety (such as any one of the anti-WTMC antibody moieties described in the “Anti-WTMC Antibody Moieties” section) that binds to a complex comprising a WT1 peptide (e.g., WT1-RMF) and a MHC class I protein (e.g., a WTMC complex). In some embodiments, the CSR is an anti-WTMC CSR (i.e., a CSR that comprises a WTMC-binding module), e.g., such as described herein. In some embodiments, the anti-WTMC CSR comprises an anti-WTMC antibody moiety (such as any one of the anti-WTMC antibody moieties described in the “Anti-WTMC Antibody Moieties” section) that binds to a complex comprising a WT1 peptide (e.g., WT1-RMF) and a MHC class I protein (e.g., a WTMC complex). In some embodiments, the WT1 peptide (e.g., WT1-RMF) comprises the amino acid sequence of SEQ ID NO: 113. In some embodiments of the anti-WTMC CSR, the antibody moiety specifically recognizes amino acid residues FPNA (SEQ ID NO: 349) of SEQ ID NO: 113. In some embodiments, the MHC class I protein is ILA-A*02:01. In some embodiments, the CSR specifically binds a WTMC that is different from the WTMC complex bound by the anti-WTMC CAR. In some embodiments, the CSR specifically binds a WTMC complex that is identical to the WTMC bound by the anti-WTMC CAR. In some embodiments, the CSR binds to a target ligand other than a WTMC complex.

In some embodiments, the anti-WTMC construct combination comprises an anti-WTMC caTCR and a CSR, wherein the anti-WTMC caTCR specifically binds a WTMC complex and the CSR specifically binds a target that is not a WTMC. In some embodiments, the CSR comprises a CD33-binding module (e.g., an anti-CD33 antibody moiety). In some embodiments, the CSR (e.g., the anti-CD33 antibody moiety of CD33-binding module of the CSR) specifically binds CD33. In some embodiments, the anti-CD33 CSR comprises a single polypeptide chain comprising: a) an extracellular domain comprising any one of the anti-CD33 antibody moieties comprising the CDR, V_{H}, and/or V_{L} sequences as described in the present application; b) a transmembrane domain, and c) an intracellular signaling domain.

In some embodiments, the anti-WTMC construct combination comprises an anti-WTMC caTCR and a CSR, wherein the anti-WTMC caTCR specifically binds a WTMC complex and the CSR specifically binds CD371, CD123, or CD15. In some embodiments, the CSR comprises a CD371-, CD123-, or CD15-binding module (e.g, an anti-CD371, anti-CD123, or anti-CD15 antibody moiety). In some embodiments, the CSR (e.g., the anti-CD371, anti-CD123, or anti-CD15 antibody moiety of CD371-, CD123-, or CD15-binding module of the CSR) specifically binds CD371, CD123, or CD15. In some embodiments, the anti-CD371, anti-CD123, or anti-CD15 CSR comprises a single polypeptide chain comprising: a) an extracellular domain comprising any one of the anti-CD371, anti-CD123, or anti-CD15 antibody moieties comprising the CDR, V_{H}, and/or V_{L} sequences as described in the art; b) a transmembrane domain, and c) an intracellular signaling domain.

In some embodiments, the anti-WTMC construct combination comprises an anti-WTMC caTCR and a CSR, wherein the anti-WTMC caTCR specifically binds a WTMC complex and the CSR specifically binds a target that is not a WTMC, wherein the CSR is a multispecific CSR. In some embodiments, the CSR comprises a CD33-binding module (e.g., any of the anti-CD33 antibody moieties comprising the CDR, V_{H}, and/or V_{L} sequences as described in the present application) and a CD371, CD123, or CD15 binding module (e.g., any of the anti-CD371, anti-CD123, or anti-CD15 antibody moieties comprising the CDR, V_{H}, and/or V_{L} sequences as described in the art). In some embodiments, the multispecific CSR is a bispecific CSR, and specifically binds both CD33 and any one of CD371, CD123, or CD15.

In some embodiments, the anti-WTMC caTCR effector cell (such as a lymphocyte, e.g., a T cell) expresses a CSR, wherein the anti-WTMC caTCR specifically binds a WTMC complex and the CSR specifically binds MUC16. In some embodiments, the CSR comprises a MUC16 binding module (e.g., an anti-MUC16 antibody moiety). In some embodiments, the CSR (e.g., the anti-MUC16 antibody moiety of MUC16 binding module of the CSR) specifically binds MUC16. In some embodiments, the anti-MUC16 CSR comprises a single polypeptide chain comprising: a) an extracellular domain comprising any one of the anti-MUC16 antibody moieties comprising the CDR, V_{H}, and/or V_{L} sequences as described in the art; b) a transmembrane module; and c) and co-stimulatory signaling module.

In some embodiments, the anti-WTMC construct combination comprises an anti-WTMC caTCR and a CSR, wherein the anti-WTMC caTCR specifically binds a WTMC complex and the CSR specifically binds HER2. In some embodiments, the CSR comprises a HER2-binding module (e.g., an anti-HER2 antibody moiety). In some embodiments, the CSR (e.g., the anti HER2 antibody moiety of HER2-binding module of the CSR) specifically binds HER2. In some embodiments, the anti-HER2 CSR comprises a single polypeptide chain comprising: a) an extracellular domain comprising any one of the anti-HER2 antibody moieties comprising the CDR, V_{H}, and/or V_{L} sequences as described in the art; b) a transmembrane module; and c) and co-stimulatory signaling module.

In some embodiments, the anti-WTMC construct combination provided herein is a CAR or a caTCR and multispecific construct (i.e., a tandem scFv, e.g., a tandem di-scFv), e.g., such as described herein, combination construct. In some embodiments, the CAR is an anti-WTMC CAR i.e., a CAR that comprises a WTMC-binding module), e.g., such as described herein. In some embodiments, the anti-WTMC CAR comprises an anti-WTMC antibody moiety (such as any one of the anti-WTMC antibody moieties described in the “Anti-WTMC Antibody Moieties” section) that binds to a complex comprising a WT1 peptide (e.g., WT1-RMF) and a MHC class I protein (e.g., a WTMC complex). In some embodiments, the caTCR is an anti-WTMC caTCR i.e., a caTCR that comprises a WTMC-binding module), e.g., such as described herein. In some embodiments, the anti-WTMC caTCR comprises an anti-WTMC antibody moiety (such as any one of the anti-WTMC antibody moieties described in the “Anti-WTMC Antibody Moieties” section) that binds to a complex comprising a WT1 peptide (e.g., WT1-RMF) and a MHC class I protein (e.g., a WTMC complex). In some embodiments, the multispecific construct is an anti-WTMC multispecific construct (i.e., an anti-WTMC tandem scFv, e.g., an anti-WTMC tandem di-scFv that comprises a WTMC-binding module), e.g., such as described herein. In some embodiments, the anti-WTMC multispecific construct comprises an anti-WTMC antibody moiety (such as any one of the anti-WTMC antibody moieties described in the “Anti-WTMC Antibody Moieties” section) that binds to a complex comprising a WT1 peptide (e.g., WT1-RMF) and a MHC class I protein (e.g., a WTMC complex). In some embodiments, the WT1 peptide (e.g., WT1-RMF) comprises the amino acid sequence of SEQ ID NO: 113. In some embodiments of the anti-WTMC multispecific construct, the antibody moiety specifically recognizes amino acid residues FPNA (SEQ ID NO: 349) of SEQ ID NO: 113. In some embodiments, the MHC class I protein is HLA-A*02:01. In some embodiments, the CAR or caTCR specifically binds a WTMC that is different from the WTMC complex bound by the anti-WTMC multispecific construct. In some embodiments, the CAR or caTCR specifically binds a WTMC complex that is identical to the WTMC bound by the anti-WTMC multispecific construct. In some embodiments, the CAR binds to a target ligand other than a WTMC complex. In some embodiments, the caTCR binds to a target ligand other than a WTMC complex.

In some embodiments, the anti-WTMC construct combination provided herein is a CSR and a CAR combination construct, wherein CSR is an anti-WTMC CSR i.e., a CSR that comprises a WTMC-binding module), e.g., such as described herein. In some embodiments, the anti-WTMC CSR comprises an anti-WTMC antibody moiety (such as any one of the anti-WTMC antibody moieties described in the “Anti-WTMC Antibody Moieties” section) that binds to a complex comprising a WT1 peptide (e.g., WT1-RMF) and a MHC class I protein (e.g., a WTMC complex). In some embodiments, the CAR is an anti-WTMC CAR i.e., a CAR that comprises a WTMC-binding module), e.g., such as described herein. In some embodiments, the anti-WTMC CAR comprises an anti-WTMC antibody moiety (such as any one of the anti-WTMC antibody moieties described in the “Anti-WTMC Antibody Moieties” section) that binds to a complex comprising a WT1 peptide (e.g., WT1-RMF) and a MHC class I protein (e.g., a WTMC complex). In some embodiments, the WT1 peptide (e.g., WT1-RMF) comprises the amino acid sequence of SEQ ID NO: 113. In some embodiments, the antibody moiety specifically recognizes amino acid residues FPNA (SEQ ID NO: 349) of SEQ ID NO: 113. In some embodiments, the MHC class I protein is HLA-A*02:01. In some embodiments, the CAR specifically binds a WTMC that is different from the WTMC complex bound by the anti-WTMC CSR. In some embodiments, the CAR specifically binds a WTMC complex that is identical to the WTMC bound by the anti-WTMC CSR. In some embodiments, the CAR binds to a target ligand other than a WTMC complex.

In some embodiments, the anti-WTMC construct combination comprises an anti-WTMC CSR and a CAR, wherein the anti-WTMC CSR specifically binds a WTMC complex and the CAR specifically binds a target that is not a WTMC. In some embodiments, the CAR specifically binds a complex comprising a WT1 peptide and a MHC class I protein. In some embodiments, the CAR comprises a WT1/MHC-binding module (e.g., an anti-WT1/MHC antibody moiety).

In some embodiments, the anti-WT1/MHC CAR (e.g., the anti-WT1/MHC antibody moiety of WT1/MHC-binding module of the anti-WT1/MHC CAR) specifically binds to a WT1/MHC complex. In some embodiments, the anti-WT1/MHC CAR comprises: a) an extracellular domain comprising any one of the anti-WT1/MHC antibody moieties comprising the CDR, V_{H}, and/or V_{L} sequences as described in the present application; b) a transmembrane domain, and c) an intracellular signaling domain.

In some embodiments, the anti-WTMC CSR is in an anti-WTMC construct combination with a CAR, wherein the anti-WTMC CSR specifically binds a WTMC complex and the CAR specifically binds CD33. In some embodiments, the CAR comprises a CD33-binding module (e.g., an anti-CD33 antibody moiety). In some embodiments, the CAR (e.g., the anti-CD33 antibody moiety of CD33-binding module of the CSR) specifically binds CD33. In some embodiments, the anti-CD33 CAR comprises: a) an extracellular domain comprising any one of the anti-CD33 antibody moieties comprising the CDR, V_{H}, and/or V_{L} sequences as described in the present application; b) a transmembrane domain, and c) an intracellular signaling domain.

In some embodiments, the anti-WTMC CSR is in an anti-WTMC construct combination with a CAR, wherein the anti-WTMC CSR specifically binds a WTMC complex and the CAR specifically binds CD371, CD123, or CD15. In some embodiments, the CAR comprises a CD371-, CD123-, or CD15-binding module (e.g., an anti-CD371, anti-CD123, or anti-CD15 antibody moiety). In some embodiments, the CAR (e.g., the anti-CD371, anti-CD123, or anti-CD15 antibody moiety of CD371-, CD123-, or CD15-binding module of the CAR) specifically binds CD371, CD123, or CD15. In some embodiments, the anti-CD371, anti-CD123, or anti-CD15 CAR comprises a) an extracellular domain comprising any one of the anti-CD371, anti-CD123, or anti-CD15 antibody moieties comprising the CDR, V_{H}, and/or V_{L} sequences as described in the art; b) a transmembrane domain, and c) an intracellular signaling domain.

In some embodiments, the anti-WTMC CSR is in an anti-WTMC construct combination with a CAR, wherein the anti-WTMC CSR specifically binds a WTMC complex and the CAR specifically binds a target that is not a WTMC, wherein the CAR is a multispecific CAR. In some embodiments, the CSR comprises a CD33-binding module (e.g., any of the anti-CD33 antibody moieties comprising the CDR, V_{H}, and/or V_{L} sequences as described in the present application) and a binding module to CD371, CD123, or CD15 (e.g., any of the anti-CD371, anti-CD123, or anti-CD15 antibody moieties comprising the CDR, V_{H}, and/or V_{L} sequences as described in the art). In some embodiments, the multispecific CAR is a bispecific CAR, and specifically binds both CD33 and any one of CD371, CD123, or CD15.

In some embodiments, the anti-WTMC CSR is in an anti-WTMC construct combination with a CAR, wherein the anti-WTMC CSR specifically binds a WTMC complex and the CAR specifically binds MUC16. In some embodiments, the CAR comprises a MUC16 binding module (e.g., an anti-MUC16 antibody moiety). In some embodiments, the CAR (e.g., the anti-MUC16 antibody moiety of MUC16 binding module of the CAR) specifically binds MUC16. In some embodiments, the anti-MUC16 CAR comprises a) an extracellular domain comprising any one of the anti-MUC16 antibody moieties comprising the CDR, V_{H}, and/or V_{L} sequences as described in the art; b) a transmembrane module; and c) and co-stimulatory signaling module.

In some embodiments, the anti-WTMC CSR is in an anti-WTMC construct combination with a CAR, wherein the anti-WTMC CSR specifically binds a WTMC complex and the CAR specifically binds HER2. In some embodiments, the CAR comprises an HER2-binding module (e.g., an anti-HER2 antibody moiety). In some embodiments, the CAR (e.g., the anti-HER2 antibody moiety of HER2-binding module of the CAR) specifically binds HER2. In some embodiments, the anti-HER2 CAR comprises a) an extracellular domain comprising any one of the anti-HER2 antibody moieties comprising the CDR, V_{H}, and/or V_{L} sequences as described in the art; b) a transmembrane module; and c) and co-stimulatory signaling module.

In some embodiments, the anti-WTMC construct combination provided herein is a TCR and CSR combination construct. In some embodiments, the TCR is an anti-WTMC TCR described herein, comprising an anti-WTMC antibody moiety (such as any one of the anti-WTMC antibody moieties described in the “Anti-WTMC Antibody Moieties” section) that binds to a complex comprising a WT1 peptide (e.g., WT1-RIMF) and a MIC class I protein (e.g., a WTMC complex). In some embodiments, the CSR is an anti-WTMC CSR described herein, comprising an anti-WTMC antibody moiety (such as any one of the anti-WTMC antibody moieties described in the “Anti-WTMC Antibody Moieties” section) that binds to a complex comprising a WT1 peptide (e.g., WT1-RMF) and a MHC class I protein (e.g., a WTMC complex). In some embodiments, the WT1 peptide (e.g., WT1-RMF) comprises the amino acid sequence of SEQ ID NO: 113. In some embodiments, the antibody moiety specifically recognizes amino acid residues FPNA (SEQ ID NO: 349) of SEQ ID NO: 113. In some embodiments, the MI-IC class I protein is HLA-A*02:01. In some embodiments, TCR specifically binds a WTMC that is different from the WTMC complex bound by the CSR. In some embodiments, the TCR specifically binds a WTMC complex that is identical to the WTMC bound by the CSR. In some embodiments, the TCR specifically binds a target that is not a WTMC.

### Effector Cells and Preparation Thereof

Provided herein is an effector cell (e.g., an immune cell, such as a T cell, e.g., an αβ T cell, a γδ T cell, a cytotoxic T cell, a helper T cell, or a natural killer T cell) that comprises, expresses, or is associated with an anti-WTMC CAR, an anti-WTMC caTCR, an anti-WTMC multispecific construct (e.g., a tandem scFv, such as a tandem di-scFv), an anti-WTMC CSR, or an anti-WTMC construct combination described herein, comprising an anti-WTMC antibody moiety (such as any one of the anti-WTMC antibody moieties described in the “Anti-WTMC Antibody Moieties” section). Such cells are also referred to as “anti-WTMC effector cells.” In some embodiments, the effector cell targets a cell surface antigen. In some embodiments, the cell surface antigen is on a cancer cell. In some embodiments, the effector cell targets a complex comprising a peptide (e.g., a WT1 peptide (e.g., WT1-RMF)) and an MHC protein on a cancer cell.

In some embodiments, the anti-WTMC effector cells (also referred to herein as “anti-WTMC immune cells” or “anti-WTMC T cells”) of the present disclosure are able to replicate in vivo, resulting in long-term persistence that can lead to sustained control of a disease associated with WT1 (such as cancer, e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)). In some embodiments, the anti-WTMC effector cell (such as a lymphocyte, e.g., a T cell) comprises (such as expresses) is an anti-WTMC CAR effector cell that comprises, expresses, or is associated with an anti-WTMC CAR described herein. In some embodiments, the anti-WTMC CAR effector cell that comprises, expresses, or is associated with an anti-WTMC CAR described herein targets a complex comprising a WT1 peptide (e.g., WT1-RMF) and a MHC class I protein (e.g., a WTMC complex), wherein the WT1 peptide (e.g., WT1-RMF) peptide comprises the amino acid sequence of SEQ ID NO: 113, and wherein the MHC class I protein is HLA-A*02:01. In some embodiments, the anti-WTMC CAR specifically recognizes amino acid residues FPNA (SEQ ID NO: 349) of SEQ ID NO: 113. In some embodiments, the anti-WTMC CAR effector cell further comprises (such as expresses) a multispecific construct. Such effector cells are referred to herein as “anti-WTMC CAR plus multispecific construct effector cells.” In some embodiments, the expression of the multispecific construct is inducible. In some embodiments, the expression of the multispecific construct is inducible upon signaling by the anti-WTMC CAR. In some embodiments, the multispecific construct is selected from the group consisting of a tandem scFv, a diabody (Db), a single chain diabody (scDb), a dual-affinity retargeting (DART) antibody, and a dual variable domain (DVD) antibody. In some embodiments, the multispecific construct is a tandem scFv. Such effector cells are also referred to herein as “anti-WTMC CAR plus tandem scFv effector cells.” In some embodiments, the tandem scFv is a tandem di-scFv, e.g., a tandem di-scFv comprising a first scFv and a second scFv, optionally connected by a peptide linker. In some embodiments, the first scFv targets a T cell surface antigen (e.g., CD3 or CD16a), a soluble immunosuppressive agent (e.g., TGF-β 1 to 4, IL-4, or IL-10), or an immune checkpoint inhibitor. In some embodiments, the second scFv targets a disease-associated antigen. In some embodiments, the disease-associated antigen is an antigen other than the target. In some embodiments, the disease-associated antigen is WT1. In some embodiments, the tandem di-scFv is an anti-WTMC anti-CD3 tandem di-scFv that comprises an antibody moiety (such as described herein) that specifically binds to a complex comprising WT1 peptide (e.g., WT1-RMF) and a MHC class I protein and a second binding moiety that specifically binds CD3. In some embodiments, the anti-WTMC anti-CD3 tandem di-scFv is encoded by a nucleic acid that is operably linked to an NFAT-derived promoter. In some embodiments, the NFAT-derived promoter is an NFAT-derived minimal promoter. In some embodiments, the anti-WTMC anti-CD3 tandem di-scFv is encoded by a nucleic acid that is operably linked to an IL-2 promoter. In some embodiments, the CAR is a bispecific CAR.

In some embodiments, the anti-WTMC CAR effector cell further comprises (such as expresses) a CSR (see, e.g., U.S. Application No. 62/490,578, filed Apr. 26, 2017, and WO2018/200583, which are incorporated by reference herein in their entirety). Such effector cells are referred to as “anti-WTMC CAR plus CSR effector cells.” In some embodiments, the CSR is an anti-WTMC CSR (i.e., a CSR that comprises a WTMC-binding module), e.g., such as described herein. In some embodiments, the anti-WTMC CSR described herein targets a complex comprising a WT1 peptide (e.g., WT1-RMF) and a MHC class I protein (e.g., a WTMC complex), wherein the WT1 peptide (e.g., WT1-RMF) comprises the amino acid sequence of SEQ ID NO: 113, and wherein the MHC class I protein is HLA-A*02:01. In some embodiments, the anti-WTMC CSR specifically recognizes amino acid residues FPNA (SEQ ID NO: 349) of SEQ ID NO: 113. In some embodiments, the CSR binds to a target ligand other than a WTMC complex. In some embodiments, the CAR is a bispecific CAR. In some embodiments, the CSR is a bispecific CSR.

In some embodiments, the anti-WTMC CAR effector cell (such as a lymphocyte, e.g., a T cell) co-expresses a CSR. In some embodiments, the anti-WTMC CAR specifically binds a WTMC complex target ligand. In some embodiments, the CSR specifically binds a WTMC complex. In some embodiments, the CSR specifically binds a WTMC that is different from the WTMC complex bound by the anti-WTMC CAR. In some embodiments, the CSR specifically binds a WTMC complex target ligand that is identical to the WTMC complex target ligand bound by the anti-WTMC CAR. In some embodiments, the CSR specifically binds a target ligand that is not a WTMC complex. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is selected from the group consisting of chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma. In some embodiments, the target ligand bound by the effector cell is expressed on a WT1-positive cancer cell.

In some embodiments, the anti-WTMC CAR effector cell (such as a lymphocyte, e.g., a T cell) expresses a CSR, wherein the anti-WTMC CAR specifically binds a WTMC complex target ligand and the CSR specifically binds a target ligand that is not a WTMC. In some embodiments, the CSR comprises a CD33-binding module (e.g., an anti-CD33 antibody moiety). In some embodiments, the CSR (e.g., the anti-CD33 antibody moiety of CD33-binding module of the CSR) specifically binds a CD33 target ligand. In some embodiments, the anti-CD33 CSR comprises a single polypeptide chain comprising: a) an extracellular domain comprising any one of the anti-CD33 antibody moieties comprising the CDR, V_{H}, and/or V_{L} sequences as described in the present application; b) a transmembrane domain, and c) an intracellular signaling domain. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is AML, myelodysplastic syndromes, chronic myelomonocytic leukemia, or acute lymphoblastic leukemia. Examples of cell lines that can be used in experiments to assess the efficacy of the anti-WTMC CAR plus anti-CD33 CSR constructs may include but are not limited to cells such as AML14, OCI-AML02, HLA*02:01+, WT1+, CD33+; and patient blood cells).

In some embodiments, the anti-WTMC CAR effector cell (such as a lymphocyte, e.g., a T cell) expresses a CSR, wherein the anti-WTMC CAR specifically binds a WTMC complex target ligand and the CSR specifically binds a MUC16 target ligand. In some embodiments, the CSR comprises a MUC16-binding module (e.g., an anti-MUC16 antibody moiety). In some embodiments, the CSR (e.g., the anti-MUC16 antibody moiety of MUC16-binding module of the CSR) specifically binds a MUC16 target ligand. In some embodiments, the anti MUC16 CSR comprises a single polypeptide chain comprising: a) an extracellular domain comprising any one of the anti-MUC16 antibody moieties comprising the CDR, V_{H}, and/or V_{L} sequences as described in the art (WO2011119979, WO2020/102555, WO2020/227538, and WO2016/149368, which are incorporated by reference herein); b) a transmembrane domain, and c) an intracellular signaling domain. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is ovarian cancer and breast cancer.

In some embodiments, the anti-WTMC CAR effector cell (such as a lymphocyte, e.g., a T cell) expresses a CSR, wherein the anti-WTMC CAR specifically binds a WTMC complex target ligand and the CSR specifically binds a target ligand that is not a WTMC, and wherein the CSR is a multispecific CSR. In some embodiments, the CSR comprises a CD33-binding module (e.g., any of the anti-CD33 antibody moieties comprising the CDR, V_{H}, and/or V_{L} sequences as described in the present application) and a binding module that specifically binds to CD371, CD123, or CD15 (e.g., any anti-CD371, -CD123, or CD15 antibody moieties comprising the CDR, V_{H}, and/or V_{L} sequences as described in the art). In some embodiments, the multispecific CSR is a bispecific CSR, and specifically binds both CD33 and a myeloid target ligand (e.g., CD371, CD123, or CD15). In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is chronic myelocytic leukemia, acute lymphoblastic leukemia (ALL), acute myeloid/myelogenous leukemia (AML), or myelodysplastic syndrome (MDS).

In some embodiments, the anti-WTMC CAR effector cell (such as a lymphocyte, e.g., a T cell) expresses a CSR, wherein the anti-WTMC CAR specifically binds a WTMC complex target ligand and the CSR specifically binds a HER2 target ligand. In some embodiments, the CSR comprises a HER2 binding module (e.g., an anti-HER2 antibody moiety). In some embodiments, the CSR (e.g., the anti-HER2 antibody moiety of a HER2 binding module of the CSR) specifically binds HER2. In some embodiments, the anti-HER2 CSR comprises a single polypeptide chain comprising: a) an extracellular domain comprising any one of the anti-HER2 antibody moieties comprising the CDR, V_{H}, and/or V_{L} sequences as described in the art (e.g., trastuzumab); b) a transmembrane module; and c) and co-stimulatory signaling module. In some embodiments, the target ligand bound by the effector cell is expressed on a cancer cell. In some embodiments, the target ligand bound by the effector cell is expressed on a WT1-positive cancer cell.

In some embodiments, the anti-WTMC effector cell (such as a lymphocyte, e.g., a T cell) comprises (such as expresses) is an anti-WTMC caTCR effector cell that comprises, expresses, or is associated with an anti-WTMC caTCR described herein. In some embodiments, the anti-WTMC caTCR described herein targets a complex comprising a WT1 peptide (e.g., WT1-RMF) and a MHC class I protein (e.g., a WTMC complex), wherein the WT1 peptide (e.g., WT1-RMF) comprises the amino acid sequence of SEQ ID NO: 113, and wherein the MHC class I protein is HLA-A*02:01. In some embodiments, the anti-WTMC caTCR specifically recognizes amino acid residues FPNA (SEQ ID NO: 349) of SEQ ID NO: 113. In some embodiments, the anti-WTMC caTCR effector cell comprises (such as expresses) a multispecific construct. Such effector cells are referred to herein as “anti-WTMC caTCR plus multispecific construct effector cells.” In some embodiments, the expression of the multispecific construct is inducible. In some embodiments, the expression of the multispecific construct is inducible upon signaling by the anti-WTMC caTCR. In some embodiments, the multispecific construct is selected from the group consisting of a tandem scFv, a diabody (Db), a single chain diabody (scDb), a dual-affinity retargeting (DART) antibody, and a dual variable domain (DVD) antibody. In some embodiments, the multispecific construct is a tandem scFv. Such effector cells are also referred to herein as “anti-WTMC caTCR plus tandem scFv effector cells.” In some embodiments the tandem scFv is a tandem di-scFv, e.g., a tandem di-scFv comprising a first scFv and a second scFv, optionally connected by a peptide linker. In some embodiments, the first scFv targets a T cell surface antigen (e.g., CD3 or CD16a), a soluble immunosuppressive agent (e.g., TGF-β 1 to 4, IL-4, or IL-10), or an immune checkpoint inhibitor. In some embodiments, the second scFv targets a disease-associated antigen. In some embodiments, the disease-associated antigen is an antigen other than the target. In some embodiments, the disease-associated antigen is WT1. In some embodiments, the tandem di-scFv is an anti-WTMC anti-CD3 tandem di-scFv that comprises an antibody moiety (such as described herein) that specifically binds to a complex comprising WT1 peptide (e.g., WT1-RMF) and a MHC class I protein and a second binding moiety that specifically binds CD3. In some embodiments, the anti-WTMC anti-CD3 tandem di-scFv is encoded by a nucleic acid that is operably linked to an NFAT-derived promoter. In some embodiments, the NFAT-derived promoter is an NFAT-derived minimal promoter. In some embodiments, the anti-WTMC anti-CD3 tandem di-scFv is encoded by a nucleic acid that is operably linked to an IL-2 promoter. In some embodiments, the caTCR is a bispecific caTCR.

In some embodiments, the anti-WTMC caTCR effector cell comprises (such as expresses) a CSR (see, e.g., US 2021/0107976, which is incorporated by reference herein in its entirety). Such effector cells are referred to as “anti-WTMC caTCR plus CSR effector cells.” In some embodiments, the CSR is an anti-WTMC CSR (i.e., a CSR that comprises a WTMC-binding module), e.g., such as described herein. In some embodiments, the CSR binds to a target ligand other than a WTMC complex. In some embodiments, the anti-WTMC caTCR plus CSR effector cell comprises (such as expresses) any of the anti-WTMC caTCR plus anti-WTMC CSR construct combinations described elsewhere herein. In some embodiments, the caTCR is a bispecific caTCR. In some embodiments, the CSR is a bispecific CSR.

In some embodiments, the effector cell (such as a lymphocyte, e.g., a T cell) expresses a caTCR and a CSR. In some embodiments, the caTCR specifically binds a WTMC complex target ligand. In some embodiments, the CSR specifically binds a WTMC complex target ligand. In some embodiments, the CSR specifically binds a WTMC complex target ligand that is different from the WTMC complex target ligand bound by the caTCR. In some embodiments, the CSR specifically binds a WTMC complex target ligand that is identical to the WTMC complex target ligand bound by the caTCR. In some embodiments, the CSR specifically binds a target ligand that is not a WTMC complex. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is selected from the group consisting of chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma. In some embodiments, the target ligand bound by the effector cell is expressed on a WT1-positive cancer cell.

In some embodiments, the anti-WTMC caTCR effector cell (such as a lymphocyte, e.g., a T cell) expresses a CSR, wherein the anti-WTMC caTCR specifically binds a WTMC complex target ligand and the CSR specifically binds a target that is not a WTMC target ligand. In some embodiments, the CSR comprises a CD33-binding module (e.g., an anti-CD33 antibody moiety). In some embodiments, the CSR (e.g., the anti-CD33 antibody moiety of CD33-binding module of the CSR) specifically binds CD33. In some embodiments, the anti-CD33 CSR comprises a single polypeptide chain comprising: a) an extracellular domain comprising any one of the anti-CD33 antibody moieties comprising the CDR, V_{H}, and/or V_{L} sequences as described in the present application; b) a transmembrane domain, and c) an intracellular signaling domain. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is chronic myelocytic leukemia, acute lymphoblastic leukemia (ALL), acute myeloid/myelogenous leukemia (AML), myelodysplastic syndrome (MDS). Examples of cell lines that can be used in experiments to assess the efficacy of the anti-WTMC caTCR plus anti-CD33 CSR constructs may include but are not limited to AML14, OCI-AML02 cells (such as HLA*02:01+, WT1+, CD33+; and leukemia patient monocytes).

In some embodiments, the effector cell (such as a lymphocyte, e.g., a T cell) comprises a CAR or a caTCR that does not target a WTMC complex and anti-WTMC multispecific construct (i.e., an anti-WTMC tandem scFv, e.g., an anti-WTMC tandem di-scFv), e.g., such as described herein. In some embodiments, the effector cell referred to as a “CAR plus anti-WTMC tandem scFv effector cell” or “caTCR plus anti-WTMC tandem scFv effector cell.”

In some embodiments, the effector cell (such as a lymphocyte, e.g., a T cell) comprises a CAR or a caTCR that does not target a WTMC complex and anti-WTMC CSR (i.e., a CSR that comprises a WTMC-binding module), e.g., such as described herein. In some embodiments, the effector cell referred to as a “CAR plus anti-WTMC CSR effector cell” or “caTCR plus anti-WTMC CSR effector cell.” In some embodiments, the CAR is a bispecific CAR. In some embodiments, the caTCR is a bispecific caTCR. In some embodiments, the CSR is a bispecific CSR.

In some embodiments, the effector cell (such as a lymphocyte, e.g., a T cell) is a CSR and expresses a CAR. In some embodiments, the CSR specifically binds a WTMC complex target ligand. In some embodiments, the CAR specifically binds a WTMC complex target ligand. In some embodiments, the CAR specifically binds a WTMC complex target ligand that is different from the WTMC complex target ligand bound by the CSR. In some embodiments, the CAR specifically binds a WTMC complex target ligand that is identical to the WTMC complex target ligand bound by the CSR. In some embodiments, the CAR specifically binds a target ligand that is not a WTMC complex. In some embodiments, the CAR is a bispecific CAR. In some embodiments, the CSR is a bispecific CSR. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is selected from the group consisting of chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma. In some embodiments, the target ligand bound by the effector cell is expressed on a WT1-positive cancer cell.

In some embodiments, the anti-WTMC CSR effector cell (such as a lymphocyte, e.g., a T cell) expresses a CAR, wherein the anti-WTMC CSR specifically binds a WTMC complex and the CAR specifically binds a target ligand that is not a WTMC. In some embodiments, the CAR specifically binds a complex comprising a WT1 peptide (e.g., WT1-RMF) and a MHC class I protein (WT1/MHC) target ligand. In some embodiments, the CAR comprises a WT1/MHC-binding module (e.g., an anti-WT1/MHC antibody moiety). In some embodiments, the anti-WT1/MHC CAR (e.g., the anti-WT1/MHC antibody moiety of WT1/MHC-binding module of the anti-WT1/MHC CAR) specifically binds to a WT1/MHC complex. In some embodiments, the anti-WT1/MHC CAR comprises: a) an extracellular domain comprising any one of the anti-WT1/MHC antibody moieties comprising the CDR, V_{H}, and/or V_{L} sequences as described in the present application; b) a transmembrane domain, and c) an intracellular signaling domain. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma.

In some embodiments, the anti-WTMC CSR effector cell (such as a lymphocyte, e.g., a T cell) expresses a CAR, wherein the anti-WTMC CSR specifically binds a WTMC complex target ligand and the CAR specifically binds a subject target ligand. In some embodiments, the CAR comprises a CD33-binding module (e.g., an anti-CD33 antibody moiety). In some embodiments, the CAR (e.g., the anti-CD33 antibody moiety of CD33-binding module of the CSR) specifically binds CD33. In some embodiments, the anti-CD33 CAR comprises: a) an extracellular domain comprising any one of the anti-CD33 antibody moieties comprising the CDR, V_{H}, and/or V_{L} sequences as described in the present application; b) a transmembrane domain, and c) an intracellular signaling domain. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is chronic myelocytic leukemia, acute lymphoblastic leukemia (ALL), acute myeloid/myelogenous leukemia (AML), or myelodysplastic syndrome (MDS).

In some embodiments, the anti-WTMC CSR effector cell (such as a lymphocyte, e.g., a T cell) expresses a CAR, wherein the anti-WTMC CSR specifically binds a WTMC complex target ligand and the CAR specifically binds a target ligand on a myeloid cell (e.g., CD371, CD123, or CD15). In some embodiments, the CAR comprises a binding module to CD371, CD123, or CD15 (e.g., an anti-CD371, -CD123, or -CD15 antibody moiety). In some embodiments, the CAR (e.g., the anti-CD371, -CD123, or -CD15 antibody moiety of CD371, CD123, or CD15-binding module of the CAR) specifically binds a CD371, CD123, or CD15 target ligand. In some embodiments, the anti-CD371, -CD123, or -CD15 CAR comprises a) an extracellular domain comprising any one of the anti-CD371, -CD123, or -CD15 antibody moieties comprising the CDR, V_{H}, and/or V_{L} sequences as described in the art; b) a transmembrane domain, and c) an intracellular signaling domain. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is chronic myelocytic leukemia, multiple myeloma (MM), acute lymphoblastic leukemia (ALL), acute myeloid/myelogenous leukemia (AML), or myelodysplastic syndrome (MDS).

In some embodiments, the anti-WTMC CSR effector cell (such as a lymphocyte, e.g., a T cell) expresses a CAR, wherein the anti-WTMC CSR specifically binds a WTMC complex target ligand and the CAR specifically binds a target ligand that is not a WTMC, and wherein the CAR is a multispecific CAR. In some embodiments, the CAR comprises a CD33-binding module (e.g., any of the anti-CD33 antibody moieties comprising the CDR, V_{H}, and/or V_{L} sequences as described in the present application) and a CD371, CD123, or CD15 binding module (e.g., any of the anti-CD371, -CD123, or -CD15 antibody moieties comprising the CDR, V_{H}, and/or V_{L} sequences as described in the art). In some embodiments, the multispecific CAR is a bispecific CAR, and specifically binds both CD33 and any one of CD371, CD123, or CD15 target ligands. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is chronic myelocytic leukemia, acute lymphoblastic leukemia (ALL), acute myeloid/myelogenous leukemia (AML), or myelodysplastic syndrome (MDS).

In some embodiments, the anti-WTMC CSR effector cell (such as a lymphocyte, e.g., a T cell) expresses a CAR, wherein the anti-WTMC CSR specifically binds a WTMC complex target ligand and the CAR specifically binds HER2 target ligand. In some embodiments, the CAR comprises a HER2-binding module (e.g., an anti-HER2 antibody moiety). In some embodiments, the CAR (e.g., the anti-HER2 antibody moiety of HER2-binding module of the CAR) specifically binds HER2. In some embodiments, the anti-HER2 CAR comprises a) an extracellular domain comprising any one of the anti-HER2 antibody moieties comprising the CDR, V_{H}, and/or V_{L} sequences as described in the art (e.g., trastuzumab); b) a transmembrane module; and c) and co-stimulatory signaling module. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is leukemia, ovarian cancer, gastrointestinal cancers, breast cancer, prostate cancer or glioblastoma.

In some embodiments, the anti-WTMC CSR effector cell (such as a lymphocyte, e.g., a T cell) expresses a CAR, wherein the anti-WTMC CSR specifically binds a WTMC complex target ligand and the CAR specifically binds MUC16 target ligand. In some embodiments, the CAR comprises a MUC16-binding module (e.g., an anti-MUC16 antibody moiety). In some embodiments, the CAR (e.g., the anti-MUC16 antibody moiety of MUC16-binding module of the CAR) specifically binds MUC16. In some embodiments, the anti-MUC16 CAR comprises a) an extracellular domain comprising any one of the anti-MUC16 antibody moieties comprising the CDR, V_{H}, and/or V_{L} sequences as described in the art (e.g., WO2011119979, WO2020/102555, WO2020/227538, and WO2016/149368, which are incorporated by reference herein); b) a transmembrane module; and c) and co-stimulatory signaling module. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is ovarian cancer or breast cancer.

In some embodiments, the effector cell (such as a lymphocyte, e.g., a T cell) is a CSR and expresses a caTCR. In some embodiments, the CSR specifically binds a WTMC complex target ligand. In some embodiments, the CAR specifically binds a WTMC complex target ligand. In some embodiments, the caTCR specifically binds a WTMC complex target ligand that is different from the WTMC complex target ligand bound by the CSR. In some embodiments, the caTCR specifically binds a WTMC complex target ligand that is identical to the WTMC complex target ligand bound by the CSR. In some embodiments, the caTCR specifically binds a target ligand that is not a WTMC complex. In some embodiments, the caTCR is a bispecific caTCR. In some embodiments, the CSR is a bispecific CSR. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is selected from the group consisting of chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma.

In some embodiments, the anti-WTMC CSR effector cell (such as a lymphocyte, e.g., a T cell) expresses a caTCR, wherein the anti-WTMC CSR specifically binds a WTMC complex and the caTCR specifically binds a target that is not a WTMC. In some embodiments, the caTCR specifically binds a complex comprising a WT1 peptide (e.g., WT1-RMF) and a MHC class I protein (WT1/MHC). In some embodiments, the caTCR comprises a WT1/MHC-binding module (e.g., an anti-WT1/MHC antibody moiety). In some embodiments, the anti-WT1/MHC caTCR (e.g., the anti-WT1/MHC antibody moiety of WT1/MHC-binding module of the anti-WT1/MHC caTCR) specifically binds to a WT1/MHC complex. In some embodiments, the anti-WT1/MHC caTCR comprises any one of the anti-WT1/MHC antibody moieties comprising the CDR, V_{H}, and/or V_{L} sequences as described in the present application. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is selected from the group consisting of chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma.

In some embodiments, the anti-WTMC CSR effector cell (such as a lymphocyte, e.g., a T cell) expresses a caTCR, wherein the anti-WTMC CSR specifically binds a WTMC complex target ligand and the caTCR specifically binds a CD33 target ligand. In some embodiments, the CAR comprises a CD33-binding module (e.g., an anti-CD33 antibody moiety). In some embodiments, the caTCR (e.g., the anti-CD33 antibody moiety of CD33-binding module of the caTCR) specifically binds CD33. In some embodiments, the anti-CD33 caTCR comprises any one of the anti-CD33 antibody moieties comprising the CDR, V_{H}, and/or V_{L} sequences as described in the present application. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is selected from the group consisting of chronic myelocytic leukemia, acute lymphoblastic leukemia (ALL), acute myeloid/myelogenous leukemia (AML), or myelodysplastic syndrome (MDS).

In some embodiments, the anti-WTMC CSR effector cell (such as a lymphocyte, e.g., a T cell) expresses a caTCR, wherein the anti-WTMC CSR specifically binds a WTMC complex target ligand and the caTCR specifically binds a CD371, CD123, or CD15 target ligand. In some embodiments, the caTCR comprises a binding module (e.g., an anti-CD371, -CD123, or -CD15 antibody moiety). In some embodiments, the caTCR (e.g., the anti-CD371, -CD123, or -CD15 antibody moiety of CD371, CD123, or CD15-binding module of the caTCR) specifically binds CD371, CD123, or CD15. In some embodiments, the anti-CD371, -CD123, or -CD15 caTCR comprises any one of the anti-CD371, -CD123, or -CD15 antibody moieties comprising the CDR, V_{H}, and/or V_{L} sequences as described in the prior art. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is selected from the group consisting of chronic myelocytic leukemia, acute lymphoblastic leukemia (ALL), acute myeloid/myelogenous leukemia (AML), or myelodysplastic syndrome (MDS).

In some embodiments, the anti-WTMC CSR effector cell (such as a lymphocyte, e.g., a T cell) expresses a caTCR, wherein the anti-WTMC CSR specifically binds a WTMC complex target ligand and the caTCR specifically binds a target ligand that is not a WTMC, and wherein the CAR is a multispecific caTCR. In some embodiments, the caTCR comprises a CD33-binding module (e.g., any of the anti-CD33 antibody moieties comprising the CDR, V_{H}, and/or V_{L} sequences as described in the present application) and a CD371, CD123, or CD15 binding module (e.g. any of the anti-CD371, CD123, or CD15 antibody moieties comprising the CDR, V_{H}, and/or V_{L} sequences as described in the art). In some embodiments, the multispecific caTCR is a bispecific caTCR, and specifically binds both CD33 and any one of CD371, CD123, or CD15 target ligands. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is selected from the group consisting of chronic myelocytic leukemia, acute lymphoblastic leukemia (ALL), acute myeloid/myelogenous leukemia (AML), or myelodysplastic syndrome (MDS).

In some embodiments, the anti-WTMC CSR effector cell (such as a lymphocyte, e.g., a T cell) expresses a caTCR, wherein the anti-WTMC CSR specifically binds a WTMC complex target ligand and the caTCR specifically binds a MUC16 target ligand. In some embodiments, the caTCR comprises a MUC16 binding module (e.g., an anti-MUC16 antibody moiety). In some embodiments, the caTCR (e.g., the anti-MUC16 antibody moiety of MUC16 binding module of the caTCR) specifically binds MUC16. In some embodiments, the anti-MUC16 caTCR comprises any one of the anti-MUC16 antibody moieties comprising the CDR, V_{H}, and/or V_{L} sequences as described in the art (e.g., WO2011119979, WO2020/102555, WO2020/227538, and WO2016/149368, which are incorporated by reference herein). In some embodiments, the target ligand bound by the effector cell is expressed on a cancer cell. In some embodiments, the target ligand bound by the effector cell is expressed on a WT1-positive cancer cell.

In some embodiments, the anti-WTMC CSR effector cell (such as a lymphocyte, e.g., a T cell) expresses a caTCR, wherein the anti-WTMC CSR specifically binds a WTMC complex target ligand and the caTCR specifically binds HER2 target ligand. In some embodiments, the caTCR comprises a HER2-binding module (e.g., an anti-HER2 antibody moiety). In some embodiments, the caTCR (e.g., the anti-HER2 antibody moiety of HER2-binding module of the caTCR) specifically binds HER2. In some embodiments, the anti-HER2 caTCR comprises any one of the anti-HER2 antibody moieties comprising the CDR, V_{H}, and/or V_{L} sequences as described in the art (e.g., trastuzumab). In some embodiments, the target ligand bound by the effector cell is expressed on a cancer cell. In some embodiments, the target ligand bound by the effector cell is expressed on a WT1-positive cancer cell.

In some embodiments, the effector cell (such as a lymphocyte, e.g., a T cell) is a CSR and expresses a TCR. In some embodiments, the CSR specifically binds a WTMC complex target ligand. In some embodiments, the TCR specifically binds a WTMC complex target ligand. In some embodiments, the TCR specifically binds a WTMC complex target ligand that is different from the WTMC complex target ligand bound by the CSR. In some embodiments, the TCR specifically binds a WTMC complex target ligand that is identical to the WTMC complex target ligand bound by the CSR. In some embodiments, the TCR specifically binds a target ligand that is not a WTMC complex. In some embodiments, the CSR is a bispecific CSR. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is selected from the group consisting of chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma.

In some embodiments, an anti-WTMC CSR effector cell (such as a lymphocyte, e.g., a T cell) expresses a TCR, wherein the anti-WTMC CSR specifically binds a WTMC complex target ligand and the TCR specifically binds a target ligand that is not a WTMC. In some embodiments, the TCR specifically binds a complex comprising a WT1 peptide (e.g., WT1-RMF) and a MHC class I protein (WT1/MHC) target ligand. In some embodiments, the TCR comprises a WT1/MHC-binding module (e.g., an anti-WT1/MHC antibody moiety). In some embodiments, the anti-WT1/MHC TCR (e.g., the anti-WT1/MHC antibody moiety of WT1/MHC-binding module of the anti-WT1/MHC TCR) specifically binds to a WT1/MHC complex. In some embodiments, the anti-WT1/MHC TCR co-expressed with the anti-WTMC CSR effector cell is a human anti-WT1/MHC TCR. In some embodiments, the target ligand bound by the effector cell is expressed on a cancer cell. In some embodiments, the target ligand bound by the effector cell is expressed on a WT1-positive cancer cell. In some embodiments, the WT1-positive cancer cell is a chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma.

Also provided herein are methods of producing the effector cells described herein.

For example, provided is a method of producing an anti-WTMC CAR effector cell, e.g., an anti-WTMC CAR immune cell or an anti-WTMC CAR T cell that comprises genetically modifying (i.e., transducing or transfecting) a cell (e.g., a T cell, such as an αβ T cell, a γδ T cell, a cytotoxic T cell, a helper T cell, or a natural killer T cell) with a nucleic acid, a set of nucleic acids, a vector, or a set of vectors encoding an anti-WTMC CAR.

In some embodiments, the method comprises genetically modifying an anti-WTMC CAR effector cell with a further nucleic acid, a set of nucleic acids, a vector, or a set of vectors encoding a multispecific construct. In some embodiments, the method of producing an anti-WTMC CAR plus multispecific construct effector cell (such as an “anti-WTMC CAR plus tandem scFv effector cell”) comprises genetically modifying (i.e., transducing or transfecting) a cell (e.g., a T cell, such as an αβ T cell, a γδ T cell, a cytotoxic T cell, a helper T cell, or a natural killer T cell) with a nucleic acid, a set of nucleic acids, a vector, or a set of vectors that encode the anti-WTMC caTCR and the multispecific construct. In some embodiments, the expression of the multispecific construct is inducible. In some embodiments, the expression of the multispecific construct is inducible upon signaling by the anti-WTMC CAR. In some embodiments, the multispecific construct is selected from the group consisting of a tandem scFv, a diabody (Db), a single chain diabody (scDb), a dual-affinity retargeting (DART) antibody, and a dual variable domain (DVD) antibody. In some embodiments, the multispecific construct is a tandem scFv. Such effector cells are also referred to herein as “anti-WTMC caTCR plus tandem scFv effector cells.” In some embodiments, the tandem scFv is a tandem di-scFv, e.g., a tandem di-scFv comprising a first scFv and a second scFv, optionally connected by a peptide linker. In some embodiments, the first scFv targets a T cell surface antigen (e.g., CD3 or CD16a), a soluble immunosuppressive agent (e.g., TGF-β 1 to 4, IL-4, or IL-10), or an immune checkpoint inhibitor. In some embodiments, the second scFv targets a disease-associated antigen. In some embodiments, the disease-associated antigen is an antigen other than the target. In some embodiments, the disease-associated antigen is WT1. In some embodiments, the tandem di-scFv is an anti-WTMC anti-CD3 tandem di-scFv that comprises an antibody moiety (such as described herein) that specifically binds a complex a complex comprising a WT1 peptide (e.g., WT1-RMF) and a MHC class I protein and a second binding moiety that specifically binds CD3. In some embodiments, the anti-WTMC anti-CD3 tandem di-scFv is encoded by a nucleic acid that is operably linked to an NFAT-derived promoter. In some embodiments, the NFAT-derived promoter is an NFAT-derived minimal promoter. In some embodiments, the anti-WTMC anti-CD3 tandem di-scFv is encoded by a nucleic acid that is operably linked to an IL-2 promoter.

In some embodiments, the method comprises genetically modifying an anti-WTMC CAR effector cell with a further nucleic acid, a set of nucleic acids, a vector, or a set of vectors encoding a CSR that comprises a target-binding domain that specifically binds to a target and a costimulatory signaling domain capable of providing a stimulatory signal to the immune cell. In some embodiments, the method of producing an anti-WTMC CAR plus CSR effector cell comprises genetically modifying (i.e., transducing or transfecting) a cell (e.g., a T cell, such as an αβ T cell, a γδ T cell, a cytotoxic T cell, a helper T cell, or a natural killer T cell) with a nucleic acid, a set of nucleic acids, a vector, or a set of vectors that encode the anti-WTMC CAR and the CSR. Further details regarding CSRs are described in U.S. Application No. 62/490,578, filed Apr. 26, 2017, and WO2018/200583, which are incorporated by reference herein in their entirety.

In some embodiments, expression of the CSR is inducible upon signaling through the anti-WTMC CAR. In some embodiments, CSR is an anti-WTMC CSR (i.e., a CSR that comprises a WTMC-binding module), e.g., such as described herein. In some embodiments, the CSR binds to a target ligand other than a WTMC complex.

Also provided is a method of producing an anti-WTMC caTCR effector cell, e.g., an anti-WTMC caTCR immune cell or an anti-WTMC caTCR T cell that comprises genetically modifying (i.e., transducing or transfecting) a cell (e.g., a T cell, such as an αβ T cell, a γδ T cell, a cytotoxic T cell, a helper T cell, or a natural killer T cell) with a nucleic acid, a set of nucleic acids, a vector, or a set of vectors encoding an anti-WTMC caTCR.

In some embodiments, the method comprises genetically modifying an anti-WTMC caTCR effector cell with a further nucleic acid, a set of nucleic acids, a vector, or a set of vectors encoding a multispecific construct. In some embodiments, the method of producing an anti-WTMC caTCR plus multispecific construct effector cell (such as an “anti-WTMC caTCR plus tandem scFv effector cell”) comprises genetically modifying (i.e., transducing or transfecting) a cell (e.g., a T cell, such as an αβ T cell, a γδ T cell, a cytotoxic T cell, a helper T cell, or a natural killer T cell) with a nucleic acid, a set of nucleic acids, a vector, or a set of vectors that encode the anti-WTMC caTCR and the multispecific construct. In some embodiments, the expression of the multispecific construct is inducible. In some embodiments, the expression of the multispecific construct is inducible upon signaling by the anti-WTMC caTCR. In some embodiments, the multispecific construct is selected from the group consisting of a tandem scFv, a diabody (Db), a single chain diabody (scDb), a dual-affinity retargeting (DART) antibody, and a dual variable domain (DVD) antibody. In some embodiments, the multispecific construct is a tandem scFv. Such effector cells are also referred to herein as “anti-WTMC caTCR plus tandem scFv effector cells.” In some embodiments, the tandem scFv is a tandem di-scFv, e.g., a tandem di-scFv comprising a first scFv and a second scFv, optionally connected by a peptide linker. In some embodiments, the first scFv targets a T cell surface antigen (e.g., CD3 or CD16a), a soluble immunosuppressive agent (e.g., TGF-β 1 to 4, IL-4, or IL-10), or an immune checkpoint inhibitor. In some embodiments, the second scFv targets a disease-associated antigen. In some embodiments, the disease-associated antigen is an antigen other than the target. In some embodiments, the disease-associated antigen is WT1. In some embodiments, the tandem di-scFv is an anti-WTMC anti-CD3 tandem di-scFv that comprises an antibody moiety (such as described herein) that specifically binds a complex comprising a WT1 peptide (e.g., WT1-RMF) and a MHC class I protein and a second binding moiety that specifically binds CD3. In some embodiments, the anti-WTMC anti-CD3 tandem di-scFv is encoded by a nucleic acid that is operably linked to an NFAT-derived promoter. In some embodiments, the NFAT-derived promoter is an NFAT-derived minimal promoter. In some embodiments, the anti-WTMC anti-CD3 tandem di-scFv is encoded by a nucleic acid that is operably linked to an IL-2 promoter.

In some embodiments, the method comprises genetically modifying an anti-WTMC caTCR effector cell with a further nucleic acid, a set of nucleic acids, a vector, or a set of vectors encoding a CSR that comprises a target-binding domain that specifically binds to a target and a costimulatory signaling domain capable of providing a stimulatory signal to the immune cell. In some embodiments, the method of producing an anti-WTMC caTCR plus CSR effector cell comprises genetically modifying (i.e., transducing or transfecting) a cell (e.g., a T cell, such as an αβ T cell, a γδ T cell, a cytotoxic T cell, a helper T cell, or a natural killer T cell) with a nucleic acid, a set of nucleic acids, a vector, or a set of vectors that encode the anti-WTMC caTCR and the CSR. Further details regarding CSRs are described in US 2021/0107976, which is incorporated by reference herein in its entirety. In some embodiments, expression of the CSR is inducible upon signaling through the anti-WTMC caTCR. In some embodiments, CSR is an anti-WTMC CSR (i.e., a CSR that comprises a WTMC-binding module), e.g., such as described herein. In some embodiments, the CSR binds to a target ligand other than a WTMC complex (e.g., CD33).

In some embodiments, the method comprises genetically modifying an effector cell (such as a lymphocyte, e.g., a T cell) that comprises nucleic acid, a set of nucleic acids, a vector, or a set of vectors encoding a CAR or a caTCR that does not target a complex comprising a WT1 peptide (e.g., WT1-RMF) and a MHC class I protein and with an additional nucleic acid, a set of nucleic acids, a vector, or a set of vectors that encodes an anti-WTMC multispecific construct (i.e., an anti-WTMC tandem scFv, e.g., an anti-WTMC tandem di-scFv), e.g., such as described herein. In some embodiments, the method comprises genetically modifying an effector cell with a nucleic acid, a set of nucleic acids, a vector, or a set of vectors that encode the CAR or caTCR that does not target a complex comprising a WT1 peptide (e.g., WT1-RMF) and a MHC class I protein and the anti-WTMC multispecific construct (i.e., an anti-WTMC tandem scFv, e.g., an anti-WTMC tandem di-scFv).

In some embodiments, the method comprises genetically modifying an effector cell (such as a lymphocyte, e.g., a T cell) that comprises nucleic acid, a set of nucleic acids, a vector, or a set of vectors encoding a CAR or a caTCR that does not target a complex comprising a WT1 peptide (e.g., WT1-RMF) and a MHC class I protein and with an additional nucleic acid, a set of nucleic acids, a vector, or a set of vectors that encodes an anti-WTMC CSR (i.e., a CSR that comprises a WTMC-binding module), e.g., such as described herein. In some embodiments, the method comprises genetically modifying an effector cell with a nucleic acid, a set of nucleic acids, a vector, or a set of vectors that encode the CAR or caTCR that does not target a complex comprising a WT1 peptide (e.g., WT1-RMF) and a MHC class I protein and the anti-WTMC CSR.

Briefly, prior to expansion and genetic modification of the cells (such as T cells), a source of cells is obtained from a subject. For example, T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available in the art may be used. In some embodiments, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as Ca²⁺-free, Mg²⁺-free PBS, PlasmaLyte A, or other saline solutions with or without buffer. Alternatively, the undesirable components of the apheresis sample may be removed and the cells directly resuspended in culture media.

In some embodiments, T cells are isolated from peripheral blood lymphocytes by lysing the red blood cells and depleting the monocytes, for example, by centrifugation through a PERCOLL^{M} gradient or by counterflow centrifugal elutriation. A specific subpopulation of T cells, such as CD3⁺, CD28⁺, CD4⁺, CD8⁺, CD45RA⁺, and CD45RO⁺ T cells, can be further isolated by positive or negative selection techniques. For example, in some embodiments, T cells are isolated by incubation with anti-CD3/anti-CD28 (i.e., 3/28)-conjugated beads, such as Dynabeads™ M-280 CD3/CD28 T, for a time period sufficient for positive selection of the desired T cells. In some embodiments, the time period is about 30 minutes. In some embodiments, the time period ranges from 30 minutes to 36 hours or longer and all integer values there between. In some embodiments, the time period is at least one, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such as in isolating tumor infiltrating lymphocytes (TIL) from tumor tissue or from immune-compromised individuals. Further, use of longer incubation times can increase the efficiency of capture of CD8⁺ T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8. In some embodiments, it may be desirable to enrich for or positively select for regulatory T cells which typically express CD4⁺, CD25⁺, CD62Lhi, GITR⁺, and FoxP3⁺. Alternatively, in some embodiments, T regulatory cells are depleted by anti-CD25 conjugated beads or other similar methods of selection.

For isolation of a desired population of cells by positive or negative selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/mL is used. In some embodiments, a concentration of about 1 billion cells/mL is used. In some embodiments, greater than about 100 million cells/mL is used. In some embodiments, a concentration of cells of about any of 10, 15, 20, 25, 30, 35, 40, 45, or 50 million cells/mL is used. In some embodiments, a concentration of cells of about any of 75, 80, 85, 90, 95, or 100 million cells/mL is used. In some embodiments, a concentration of about 125 or about 150 million cells/mL is used. Using high concentrations can result in increased cell yield, cell activation, and cell expansion. Further, use of high cell concentrations allows more efficient capture of cells that may weakly express target antigens of interest, such as CD4-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In some embodiments, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in some embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

Whether prior to or after genetic modification of the T cells to express, e.g., an anti-WTMC CAR or an anti-WTMC caTCR, optionally with a CSR (such as an anti-WTMC CSR) or a tandem scFv (such as an anti-WTMC tandem scFv), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the genetically modified cells (such as T cells, such as αβ T cells, T6 T cells, cytotoxic T cells, helper T cells, or natural killer T cells) described herein are expanded by contact with a surface having attached thereto an agent that stimulates a CD3/TCR complex associated signal and a ligand that stimulates a co-stimulatory molecule on the surface of the T cells. In particular, T cell populations may be stimulated, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

### Immunoconjugates and Preparation Thereof

The anti-WTMC constructs in some embodiments comprise an immunoconjugate comprising an anti-WTMC antibody moiety (such as any one of the anti-WTMC antibody moieties described in the “Anti-WTMC Antibody Moieties” section), attached to an effector molecule (also referred to herein as an “anti-WTMC immunoconjugate”). In some embodiments, the effector molecule is a therapeutic agent, such as a viral therapeutic agent, which is either cytotoxic, cytostatic or otherwise provides some therapeutic benefit. In some embodiments, the effector molecule is a label, which can generate a detectable signal, either directly or indirectly.

In some embodiments, there is provided an anti-WTMC immunoconjugate comprising an anti-WTMC antibody moiety and a therapeutic agent (also referred to herein as an “antibody-drug conjugate”, or “ADC”). In some embodiments, the therapeutic agent is a toxin that is either cytotoxic, cytostatic or otherwise prevents or reduces the ability of the target cells to divide. The use of ADCs for the local delivery of cytotoxic or cytostatic agents, i.e., drugs to kill or inhibit tumor cells in the treatment of cancer (Syrigos and Epenetos, Anticancer Research 19:605-614 (1999); Niculescu-Duvaz and Springer, Adv. Drg. Del. Rev. 26:151-172 (1997); U.S. Pat. No. 4,975,278) allows targeted delivery of the drug moiety to target cells, and intracellular accumulation therein, where systemic administration of these unconjugated therapeutic agents may result in unacceptable levels of toxicity to normal cells as well as the target cells sought to be eliminated (Baldwin et al., Lancet (Mar. 15, 1986):603-605 (1986); Thorpe, (1985) “Antibody Carriers Of Cytotoxic Agents In Cancer Therapy: A Review,” in Monoclonal Antibodies '84: Biological And Clinical Applications, A. Pinchera et al. (eds.), pp. 475-506). Maximal efficacy with minimal toxicity is sought thereby. Importantly, since most normal cells do not present the WTMC on their surface, they cannot bind the anti-WTMC immunoconjugate, and are protected from the killing effect of the toxin or other therapeutic agents.

Therapeutic agents used in anti-WTMC immunoconjugates include, for example, daunomycin, doxorubicin, methotrexate, and vindesine (Rowland et al., Cancer Immunol. Immunother. 21:183-187 (1986)). Toxins used in anti-WTMC immunoconjugates include bacterial toxins such as diphtheria toxin, plant toxins such as ricin, small molecule toxins such as geldanamycin (Mandler et al., J. Nat. Cancer Inst. 92(19):1573-1581 (2000); Mandler et al., Bioorganic & Med. Chem. Letters 10:1025-1028 (2000); Mandler et al., Bioconjugate Chem. 13:786-791 (2002)), maytansinoids (EP 1391213; Liu et al., Proc. Natl. Acad. Sci. USA 93:8618-8623 (1996)), and calicheamicin (Lode et al., Cancer Res. 58:2928 (1998); Hinman et al., Cancer Res. 53:3336-3342 (1993)). The toxins may exert their cytotoxic and cytostatic effects by mechanisms including tubulin binding, DNA binding, or topoisomerase inhibition. Some cytotoxic drugs tend to be inactive or less active when conjugated to large antibodies or protein receptor ligands.

Enzymatically active toxins and fragments thereof that can be used include, for example, diphtheria A chain, nonbinding active fragments of diphtheria toxin, exotoxin A chain (from Pseudomonas aeruginosa), ricin A chain, abrin A chain, modeccin A chain, α-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. See, e.g., WO 93/21232 published Oct. 28, 1993.

Anti-WTMC immunoconjugates of an anti-WTMC antibody moiety and one or more small molecule toxins, such as a calicheamicin, maytansinoids, dolastatins, aurostatins, a trichothecene, and CC1065, and the derivatives of these toxins that have toxin activity, are also contemplated herein.

In some embodiments, there is provided an anti-WTMC immunoconjugate comprising a therapeutic agent that has an intracellular activity. In some embodiments, the anti-WTMC immunoconjugate is internalized and the therapeutic agent is a cytotoxin that blocks the protein synthesis of the cell, therein leading to cell death. In some embodiments, the therapeutic agent is a cytotoxin comprising a polypeptide having ribosome-inactivating activity including, for example, gelonin, bouganin, saporin, ricin, ricin A chain, bryodin, diphtheria toxin, restrictocin, Pseudomonas exotoxin A and variants thereof. In some embodiments, where the therapeutic agent is a cytotoxin comprising a polypeptide having a ribosome-inactivating activity, the anti-WTMC immunoconjugate must be internalized upon binding to the target cell in order for the protein to be cytotoxic to the cells.

In some embodiments, there is provided an anti-WTMC immunoconjugate comprising a therapeutic agent that acts to disrupt DNA. In some embodiments, the therapeutic agent that acts to disrupt DNA is, for example, selected from the group consisting of enediyne (e.g., calicheamicin and esperamicin) and non-enediyne small molecule agents (e.g., bleomycin, methidiumpropyl-EDTA-Fe(II)).

The present application further contemplates an anti-WTMC immunoconjugate formed between the anti-WTMC antibody moiety and a compound with nucleolytic activity (e.g., a ribonuclease or a DNA endonuclease such as a deoxyribonuclease; DNase).

In some embodiments, the anti-WTMC immunoconjugate comprises an agent that acts to disrupt tubulin. Such agents may include for example, rhizoxin/maytansine, paclitaxel, vincristine and vinblastine, colchicine, auristatin dolastatin 10 MMAE, and peloruside A.

In some embodiments, the anti-WTMC immunoconjugate comprises an alkylating agent including, for example, Asaley NSC 167780, AZQ NSC 182986, BCNU NSC 409962, Busulfan NSC 750, carboxyphthalatoplatinum NSC 271674, CBDCA NSC 241240, CCNU NSC 79037, CHIP NSC 256927, chlorambucil NSC 3088, chlorozotocin NSC 178248, cis-platinum NSC 119875, clomesone NSC 338947, cyanomorpholinodoxorubicin NSC 357704, cyclodisone NSC 348948, dianhydrogalactitol NSC 132313, fluorodopan NSC 73754, hepsulfam NSC 329680, hycanthone NSC 142982, melphalan NSC 8806, methyl CCNU NSC 95441, mitomycin C NSC 26980, mitozolamide NSC 353451, nitrogen mustard NSC 762, PCNU NSC 95466, piperazine NSC 344007, piperazinedione NSC 135758, pipobroman NSC 25154, porfiromycin NSC 56410, spirohydantoin mustard NSC 172112, teroxirone NSC 296934, tetraplatin NSC 363812, thio-tepa NSC 6396, triethylenemelamine NSC 9706, uracil nitrogen mustard NSC 34462, and Yoshi-864 NSC 102627.

In some embodiments, the anti-WTMC immunoconjugate comprises a highly radioactive atom. A variety of radioactive isotopes are available for the production of radioconjugated antibodies. Examples include ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb and radioactive isotopes of Lu.

In some embodiments, the anti-WTMC antibody moiety can be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

In some embodiments, an anti-WTMC immunoconjugate may comprise an anti-WTMC antibody moiety conjugated to a prodrug-activating enzyme. In some such embodiments, a prodrug-activating enzyme converts a prodrug to an active drug, such as an anti-viral drug. Such anti-WTMC immunoconjugates are useful, in some embodiments, in antibody-dependent enzyme-mediated prodrug therapy (“ADEPT”). Enzymes that may be conjugated to an antibody include, but are not limited to, alkaline phosphatases, which are useful for converting phosphate-containing prodrugs into free drugs; arylsulfatases, which are useful for converting sulfate-containing prodrugs into free drugs; proteases, such as Serratia protease, thermolysin, subtilisin, carboxypeptidases and cathepsins (such as cathepsins B and L), which are useful for converting peptide-containing prodrugs into free drugs; D-alanylcarboxypeptidases, which are useful for converting prodrugs that contain D-amino acid substituents; carbohydrate-cleaving enzymes such as β-galactosidase and neuraminidase, which are useful for converting glycosylated prodrugs into free drugs; β-lactamase, which is useful for converting drugs derivatized with β-lactams into free drugs; and penicillin amidases, such as penicillin V amidase and penicillin G amidase, which are useful for converting drugs derivatized at their amine nitrogens with phenoxyacetyl or phenylacetyl groups, respectively, into free drugs. In some embodiments, enzymes may be covalently bound to antibody moieties by recombinant DNA techniques well known in the art. See, e.g., Neuberger et al., Nature 312:604-608 (1984).

In some embodiments, the therapeutic portion of the anti-WTMC immunoconjugates may be a nucleic acid. Nucleic acids that may be used include, but are not limited to, anti-sense RNA, genes or other polynucleotides, including nucleic acid analogs such as thioguanine and thiopurine.

The present application further provides anti-WTMC immunoconjugates comprising an anti-WTMC antibody moiety attached to an effector molecule, wherein the effector molecule is a label, which can generate a detectable signal, indirectly or directly. These anti-WTMC immunoconjugates can be used for research or diagnostic applications, such as for the in vivo detection of cancer. The label is preferably capable of producing, either directly or indirectly, a detectable signal. For example, the label may be radio-opaque or a radioisotope, such as ³H, ¹⁴C, ³²P, ³⁵, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, β-galactosidase or horseradish peroxidase; an imaging agent; or a metal ion. In some embodiments, the label is a radioactive atom for scintigraphic studies, for example ⁹⁹Tc or ¹²³I, or a spin label for nuclear magnetic resonance (NMR) imaging (also known as magnetic resonance imaging, MRI), such as zirconium-89, iodine-123, iodine-131, indium-111, fluorine-19, carbon-13, nitrogen-15, oxygen-17, gadolinium, manganese or iron. Zirconium-89 may be complexed to various metal chelating agents and conjugated to antibodies, e.g., for PET imaging (WO 2011/056983).

In some embodiments, the anti-WTMC immunoconjugate is detectable indirectly. For example, a secondary antibody that is specific for the anti-WTMC immunoconjugate and contains a detectable label can be used to detect the anti-WTMC immunoconjugate.

Thus, for example, in some embodiments, there is provided an anti-WTMC immunoconjugate comprising a) an anti-WTMC antibody moiety, and b) an effector molecule. In some embodiments, the WT1 peptide (e.g., WT1-RMF) comprises SEQ ID NO: 113. In some embodiments, the antibody moiety specifically recognizes amino acid residues FPNA (SEQ ID NO: 349) of SEQ ID NO: 113. In some embodiments, the MHC class I protein is HLA-A02. In some embodiments, the MHC class I protein is HLA-A*02:01. In some embodiments, the effector molecule is covalently attached to the anti-WTMC antibody moiety. In some embodiments, the effector molecule is a therapeutic agent selected, for example, from the group consisting of a drug, a toxin, a radioisotope, a protein, a peptide, and a nucleic acid. In some embodiments, the effector molecule is a viral therapeutic agent. In some embodiments, the viral therapeutic agent is a highly radioactive atom selected, for example, from the group consisting of ²¹¹At, ¹³¹I, ¹²⁵I, ⁹⁰Y, ¹⁸⁶Re, ¹⁸⁸Re, ¹⁵³Sm, ²¹²Bi, ³²P, and ²¹²Pb. In some embodiments, the effector molecule is a label that can generate a detectable signal, either directly or indirectly. In some embodiments, the label is a radioisotope selected, for example, from the group consisting of ³H, ¹⁴C, ³²P, ³⁵S, ¹²³I, ¹²⁵I, and ¹³¹I. In some embodiments, the anti-WTMC antibody moiety is an scFv. In some embodiments, the anti-WTMC antibody moiety is human, humanized, or semi-synthetic. In some embodiments, the anti-WTMC antibody moiety cross-reacts with at least one (such as at least any of 2, 3, 4, 5, or 6) complex comprising the MHC class I protein and a variant of the WT1 peptide having one amino acid substitution (such as a conservative amino acid substitution). In some embodiments, the anti-WTMC antibody moiety cross-reacts with at least one (such as at least any of 2, 3, 4, or 5) complex comprising the WT1 peptide and a different subtype of the MHC class I protein.

In some embodiments, there is provided an anti-WTMC immunoconjugate comprising a) an anti-WTMC antibody moiety according to any one of the anti-WTMC antibody moieties described in the “Anti-WTMC Antibody Moiety” Section, and b) an effector molecule.

The anti-WTMC immunoconjugates may be prepared using any methods known in the art. See, e.g., WO 2009/067800, WO 2011/133886, and U.S. Patent Application Publication No. 2014322129, incorporated by reference herein in their entirety.

The anti-WTMC antibody moiety of an anti-WTMC immunoconjugate may be “attached to” the effector molecule by any means by which the anti-WTMC antibody moiety can be associated with, or linked to, the effector molecule. For example, the anti-WTMC antibody moiety of an anti-WTMC immunoconjugate may be attached to the effector molecule by chemical or recombinant means. Chemical means for preparing fusions or conjugates are known in the art and can be used to prepare the anti-WTMC immunoconjugate. The method used to conjugate the anti-WTMC antibody moiety and effector molecule must be capable of joining the binding protein with the effector molecule without interfering with the ability of the binding protein to bind to the antigen on the target cell.

The anti-WTMC antibody moiety of an anti-WTMC immunoconjugate may be linked indirectly to the effector molecule. For example, the anti-WTMC antibody moiety of an anti-WTMC immunoconjugate may be directly linked to a liposome containing the effector molecule of one of several types. The effector molecule(s) and/or the anti-WTMC antibody moiety may also be bound to a solid surface.

In some embodiments, the anti-WTMC antibody moiety of an anti-WTMC immunoconjugate and the effector molecule are both proteins and can be conjugated using techniques well known in the art. There are several hundred crosslinkers available that can conjugate two proteins. (See for example “Chemistry of Protein Conjugation and Crosslinking”. 1991, Shans Wong, CRC Press, Ann Arbor). The crosslinker is generally chosen based on the reactive functional groups available or inserted on the anti-WTMC antibody moiety and/or effector molecule. In addition, if there are no reactive groups, a photoactivatible crosslinker can be used. In certain instances, it may be desirable to include a spacer between the anti-WTMC antibody moiety and the effector molecule. Crosslinking agents known to the art include the homobifunctional agents: glutaraldehyde, dimethyladipimidate and Bis(diazobenzidine) and the heterobifunctional agents: m Maleimidobenzoyl-N-Hydroxysuccinimide and Sulfo-m Maleimidobenzoyl-N-Hydroxysuccinimide.

In some embodiments, the anti-WTMC antibody moiety of an anti-WTMC immunoconjugate may be engineered with specific residues for chemical attachment of the effector molecule. Specific residues used for chemical attachment of a molecule known to the art include lysine and cysteine. The crosslinker is chosen based on the reactive functional groups inserted on the anti-WTMC antibody moiety, and available on the effector molecule.

An anti-WTMC immunoconjugate may also be prepared using recombinant DNA techniques. In such a case a DNA sequence encoding the anti-WTMC antibody moiety is fused to a DNA sequence encoding the effector molecule, resulting in a chimeric DNA molecule. The chimeric DNA sequence is transfected into a host cell that expresses the fusion protein. The fusion protein can be recovered from the cell culture and purified using techniques known in the art.

Examples of attaching an effector molecule, which is a label, to the binding protein include the methods described in Hunter, et al., Nature 144:945 (1962); David, et al., Biochemistry 13:1014 (1974); Pain, et al., J. Immunol. Meth. 40:219 (1981); Nygren, J. Histochem. and Cytochem. 30:407 (1982); Wensel and Meares, Radioimmunoimaging And Radioimmunotherapy, Elsevier, N.Y. (1983); and Colcher et al., “Use of monoclonal antibodies as radiopharmaceuticals for the localization of human carcinoma xenografts in athymic mice”, Meth. Enzymol., 121:802-16 (1986).

The radio- or other labels may be incorporated in the immunoconjugate in known ways. For example, the peptide may be biosynthesized or may be synthesized by chemical amino acid synthesis using suitable amino acid precursors involving, for example, fluorine-19 in place of hydrogen. Labels such as ⁹⁹Tc or ¹²³I, ¹⁸⁶Re, ¹⁸⁸Re and ¹¹¹In can be attached via a cysteine residue in the peptide. Yttrium-90 can be attached via a lysine residue. The IODOGEN method (Fraker et al., Biochem. Biophys. Res. Commun. 80:49-57 (1978)) can be used to incorporate iodine-123. “Monoclonal Antibodies in Immunoscintigraphy” (Chatal, CRC Press 1989) describes other methods in detail.

Immunoconjugates of the antibody moiety and a cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene tnaminepentaacetic acid (MX-DTPA) is an exemplary chelating agent for conjugation of radionucleotide to the antibody. See, e.g., WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

The anti-WTMC immunoconjugates of the present application expressly contemplate, but are not limited to, ADC prepared with cross-linker reagents: BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL, U.S.A). See pages 467-498, 2003-2004 Applications Handbook and Catalog.

### Anti-WTMC Constructs Comprising Anti-WTMC Antibody Moiety Sequence Variants

In some embodiments, anti-WTMC constructs of the present application comprise variants (such as amino acid sequence variants) of the anti-WTMC antibody moieties (such as any one of the anti-WTMC antibody moieties described in the “Anti-WTMC Antibody Moieties” section). For example, it may be desirable to improve the binding affinity and/or other biological properties of the anti-WTMC antibody moiety of an anti-WTMC construct. Amino acid sequence variants of an anti-WTMC antibody moiety may be prepared by introducing appropriate modifications into the nucleotide sequence encoding the antibody moiety, or by peptide synthesis. Such modifications include, for example, deletions from, insertions into, and/or substitutions of residues within the amino acid sequences of the anti-WTMC antibody moiety. Any combination of deletion(s), insertion(s), and substitution(s) can be made to arrive at the final anti-WTMC antibody moiety, provided that the final antibody moiety possesses the desired characteristics, e.g., binding to a WTMC complex.

In some embodiments, an anti-WTMC antibody moiety sequence variant comprises one or more amino acid substitutions. Sites of interest for substitutional mutagenesis include the CDRs and/or the framework regions (FRs). Amino acid substitutions may be introduced into an anti-WTMC antibody moiety of interest and the products screened for a desired activity, e.g., retained/improved binding to a WTMC complex, decreased immunogenicity, or improved ADCC or CDC, etc. Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an anti-WTMC antibody moiety with an N-terminal methionyl residue. Other insertional variants of the anti-WTMC antibody moiety include the fusion to the N- or C-terminus of the antibody moiety to an enzyme (e.g., for ADEPT) or a polypeptide which increases the serum half-life of the anti-WTMC antibody moiety.

In some embodiments, an anti-WTMC antibody moiety sequence variant comprises one or more conservative amino acid substitutions, as shown in Table E below.

TABLE E
Conservative Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties:
a) Hydrophobic: Norleucine, Met, Aia, Vai, Leu, Ile;
b) Neutral hydrophilic: Cys, Ser, Thr, Asn, Gin;
c) Acidic: Asp, Glu;
d) Basic: His, Lys, Arg;
e) Residues that influence chain orientation: Gly, Pro;
f) Aromatic: Trp, Tyr, Phe.

Non-conservative substitutions entail exchanging a member of one of these classes for another class.

An exemplary substitutional variant is an affinity matured antibody moiety, which may be conveniently generated, e.g., using phage display-based affinity maturation techniques. Briefly, one or more CDR residues are mutated and the variant antibody moieties displayed on phage and screened for a particular biological activity (e.g., binding affinity). Alterations (e.g., substitutions) may be made in CDRs, e.g., to improve antibody moiety affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or specificity determining residues (SDRs), with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).)

In some embodiments, one or more CDR sequences provided herein is either unaltered, or contains no more than one, two, three, four, or five amino acid substitutions. In some embodiments a V_{H} and/or V_{L} sequence provided herein is either unaltered, or contains no more than one, two, three, four, or five amino acid substitutions. In some embodiments one or more CDR sequences within a V_{H} and/or V_{L} sequence provided herein is either unaltered, or contains no more than one, two, three, four, or five amino acid substitutions.

Diversity may be introduced into the variable genes chosen for maturation by any of a variety of methods (e.g., error-prone PCR, chain shuffling, or oligonucleotide-directed mutagenesis). A secondary library is then created. The library is then screened to identify any antibody moiety variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. HC-CDR3 and LC-CDR3 in particular are often targeted.

The anti-WTMC antibodies or anti-WTMC antibody moieties may also be identified by screening combinatorial libraries for antibodies with the desired activity or activities. For example, a variety of methods are known in the art for generating polypeptide display libraries and screening such libraries for antibodies possessing the desired binding characteristics. Such methods are reviewed, e.g., in Hoogenboom et al., Methods in Molecular Biology 178:1-37 (O'Brien et al., ed., Human Press, Totowa, N.J., 2001) and further described, e.g., in McCafferty et al., Nature 348:552-554; Clackson et al., Nature 352: 624-628 (1991); Marks et al., J. Mol. Biol. 222: 581-597 (1992); Marks and Bradbury, Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naïve repertoire can be cloned (e.g., from human) to provide a single source of antibodies to a wide range of non-self as well as self-antigens without any immunization as described by Griffiths et al., EMBO J, 12: 725-734 (1993). Finally, naïve libraries can also be made synthetically by cloning unrearranged V-gene segments from stem cells and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Anti-WTMC antibody moiety sequence variants can be prepared using phage display to screen libraries for antibodies specific to WT1 peptide (e.g., WT1-RMF). The library can be a human scFv phage display library having a diversity of at least 1×10⁹ (such as at least about any one of 1×10⁹, 2.5×10⁹, 5×10⁹, 7.5×10⁹, 1×10¹⁰, 2.5×10¹⁰, 5×10¹⁰, 7.5×10¹⁰, or 1×10¹¹) unique human antibody fragments. In some embodiments, the library is a naïve human library constructed from DNA extracted from human PMBCs and spleens from healthy donors, encompassing all human heavy and light chain subfamilies. In some embodiments, the library is a naïve human library constructed from DNA extracted from PBMCs isolated from patients with various diseases, such as patients with autoimmune diseases, cancer patients, and patients with infectious diseases. In some embodiments, the library is a semi-synthetic human library, wherein heavy chain CDR3 is completely randomized, with all amino acids (with the exception of cysteine) equally likely to be present at any given position (see, e.g., Hoet, R. M. et al., Nat. Biotechnol. 23(3):344-348, 2005). In some embodiments, the heavy chain CDR3 of the semi-synthetic human library has a length from about 5 to about 24 amino acids (such as about any of 5, 6, 7, 8, 9, 10, 11, 12, 13, 14, 15, 16, 17, 18, 19, 20, 21, 22, 23, or 24 amino acids). In some embodiments, the library is a non-human phage display library.

Phage clones that bind to WT1 peptide (e.g., WT1-RMF) with high affinity can be selected by iterative binding of phage to WT1 peptide (e.g., WT1-RMF), which is bound to a solid support (such as, for example, beads for solution panning or mammalian cells for cell panning), followed by removal of non-bound phage and by elution of specifically bound phage. In an example of solution panning, the WT1 peptide (e.g., WT1-RMF) can be biotinylated for immobilization to a solid support. The biotinylated WT1 peptide (e.g., WT1-RMF) is mixed with the phage library and a solid support, such as streptavidin-conjugated Dynabeads™ M-280, and then WT1 peptide (e.g., WT1-RMF) -phage-bead complexes are isolated. The bound phage clones are then eluted and used to infect an appropriate host cell, such as E. coli XL1-Blue, for expression and purification.

In another example of cell panning, mammalian cells expressing cell surface-bound WT1 peptide (e.g., WT1-RMF) (such as T2 cells expressing human WT1 peptide (e.g., WT1-RMF), optionally wherein the WT1 peptide (e.g., WT1-RMF) lacks a signal peptide of the WT1 peptide (e.g., WT1-RMF) epitope) are mixed with the phage library, after which the cells are collected and the bound clones are eluted and used to infect an appropriate host cell for expression and purification. The panning can be performed for multiple (such as about any of 2, 3, 4, 5, 6 or more) rounds via solution panning, cell panning, or a combination of both, to enrich for phage clones binding specifically to the WT1 peptide (e.g., WT1-RMF). Enriched phage clones can be tested for specific binding to WT1 peptide (e.g., WT1-RMF) by any methods known in the art, including for example ELISA and FACS.

A useful method of identification of residues or regions of an anti-WTMC antibody moiety that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody moiety with antigen is affected. Further substitutions may be introduced at the amino acid locations demonstrating functional sensitivity to the initial substitutions. Alternatively, or additionally, a crystal structure of an antigen-antibody moiety complex can be determined to identify contact points between the antibody moiety and antigen. Such contact residues and neighboring residues may be targeted or eliminated as candidates for substitution. Variants may be screened to determine whether they contain the desired properties.

An anti-WTMC antibody moiety provided herein may additionally comprise one or more peptide tag sequences, peptide linker sequences (including self-cleaving linkers), cleavage sites, or other peptide sequences (e.g., signal peptides). An exemplary signal peptide sequence is METDTLLLWVLLLWVPGSTG (SEQ ID NO: 280). Exemplary peptide linker sequences, cleavage sites, and peptide tag sequences are shown in Tables F-1 and F-2 below.

TABLE F-1
Exemplary Peptide Linkers and Cleavage Sites

| | | | |
|---|---|---|---|
| SRGGGGSGGGGSGGGG | GGGGS | SGGG | GGSGGSGGSGGS |
| SLEMA | (SEQ ID NO: 250) | (SEQ ID NO: 251) | (SEQ ID NO: 252) |
| (SEQ ID NO: 249) | | | |
| RAKRS | GGGGSGGGGS | GSGS | GGSG |
| (SEQ ID NO: 253) | (SEQ ID NO: 254) | (SEQ ID NO: 255) | (SEQ ID NO: 256) |
| GSGAPVKQTLNFDLLK | GGGGSGGGGSGGGGS | GSGSGS | GGSGGGSG |
| LAGDVESNPGP | (SEQ ID NO: 258) | (SEQ ID NO: 259) | (SEQ ID NO: 260) |
| (SEQ ID NO: 257) | | | |
| RAKRSGSGAPVKQTLN | AAATG | GSGSGSGS | GGSGGGSGGGSG |
| FDLLKLAGDVESNPGP | (SEQ ID NO: 262) | (SEQ ID NO: 263) | (SEQ ID NO: 264) |
| (SEQ ID NO: 261) | | | |
| GSGATNFSLLKQAGDV | TPLGDTTHTSG | GSGSGSGSGS | RAKRSGSGATNFSLLK |
| EENPGP | (SEQ ID NO: 266) | (SEQ ID NO: 267) | QAGDVEENPGP |
| (SEQ ID NO: 265) | | | (SEQ ID NO: 268) |
| AAA | GGSGGS | GSRGGGGSGGGGSGGG | GGSG |
| (SEQ ID NO: 269) | (SEQ ID NO: 270) | GSLEMA | (SEQ ID NO: 272) |
| | | (SEQ ID NO: 271) | |
| GGSGGSGGS | GSGEGRGSLLTCGDVE | | |
| (SEQ ID NO: 273) | ENPGP | | |
| | (SEQ ID NO: 329) | | |

TABLE F-2
Exemplary Peptide Tags

| | |
|---|---|
| EQKLISEEDL | HHHHHH |
| (SEQ ID NO: 274) | (SEQ ID NO: 277) |
| DYKDHDGDYKDHDIDYKDDDDK | YPYDVPDYA |
| (SEQ ID NO: 275) | (SEQ ID NO: 278) |
| DYKDDDDK | YPYDVPDYAS |
| (SEQ ID NO 276) | (SEQ ID NO: 279) |

### Anti-WTMC Constructs Comprising Fc Region Variants

In some embodiments, one or more amino acid modifications may be introduced into the Fc region of a full-length anti-WTMC antibody provided herein (such as any one of the anti-WTMC antibody moieties described in the “Anti-WTMC Antibody Moieties” section), thereby generating an Fc region variant. In some embodiments, the Fc region variant has enhanced antibody dependent cellular cytotoxicity (ADCC) effector function, often related to binding to Fc receptors (FcRs). In some embodiments, the Fc region variant has decreased ADCC effector function. There are many examples of changes or mutations to Fc sequences that can alter effector function. For example, WO 00/42072 and Shields et al. J Biol. Chem. 9(2): 6591-6604 (2001) describe antibody variants with improved or diminished binding to FcRs. The contents of those publications are specifically incorporated herein by reference.

Antibody-Dependent Cell-Mediated Cytotoxicity (ADCC) is a mechanism of action of therapeutic antibodies against tumor cells. ADCC is a cell-mediated immune defense whereby an effector cell of the immune system actively lyses a target cell (e.g., a cancer cell), whose membrane-surface antigens have been bound by specific antibodies (e.g., an anti-WTMC antibody). The typical ADCC involves activation of NK cells by antibodies. An NK cell expresses CD16 which is an Fc receptor. This receptor recognizes, and binds to, the Fc portion of an antibody bound to the surface of a target cell. The most common Fc receptor on the surface of an NK cell is called CD16 or FcγRIII. Binding of the Fc receptor to the Fc region of an antibody results in NK cell activation, release of cytolytic granules and consequent target cell apoptosis. The contribution of ADCC to tumor cell killing can be measured with a specific test that uses NK-92 cells that have been transfected with a high-affinity FcR. Results are compared to wild-type NK-92 cells that do not express the FcR.

In some embodiments, the present application contemplates an anti-WTMC construct variant comprising an FC region that possesses some but not all effector functions, which makes it a desirable candidate for applications in which the half-life of the anti-WTMC construct in vivo is important yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTI™ non-radioactive cytotoxicity assay for flow cytometry (CellTechnology, Inc. Mountain View, Calif; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat7Acad. Sci. USA 95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001)).

In some embodiments, there is provided an anti-WTMC construct (e.g., a full-length anti-WTMC antibody) variant comprising a variant Fc region comprising one or more amino acid substitutions which improve ADCC. In some embodiments, the variant Fc region comprises one or more amino acid substitutions which improve ADCC, wherein the substitutions are at positions 298, 333, and/or 334 of the variant Fc region (EU numbering of residues). In some embodiments, the anti-WTMC construct (e.g., full-length anti-WTMC antibody) variant comprises the following amino acid substitution in its variant Fc region: S298A, E333A, and K334A.

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al., J. Immunol. 164: 4178-4184 (2000).

In some embodiments, there is provided an anti-WTMC construct (e.g., a full-length anti-WTMC antibody) variant comprising a variant Fc region comprising one or more amino acid substitutions which increase half-life and/or improve binding to the neonatal Fc receptor (FcRn). Antibodies with increased half-lives and improved binding to FcRn are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

Anti-WTMC constructs (such as full-length anti-WTMC antibodies) comprising any of the Fc variants described herein, or combinations thereof, are contemplated.

### Glycosylation Variants

In some embodiments, an anti-WTMC construct provided herein is altered to increase or decrease the extent to which the anti-WTMC construct is glycosylated. Addition or deletion of glycosylation sites to an anti-WTMC construct may be conveniently accomplished by altering the amino acid sequence of the anti-WTMC construct or polypeptide portion thereof such that one or more glycosylation sites is created or removed.

Where the anti-WTMC construct comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-WTMC construct of the present application may be made in order to create anti-WTMC construct variants with certain improved properties.

In some embodiments, anti-WTMC construct (such as full-length anti-WTMC antibody) variants are provided comprising an Fc region wherein a carbohydrate structure attached to the Fc region has reduced fucose or lacks fucose, which may improve ADCC function. Specifically, anti-WTMC constructs are contemplated herein that have reduced fusose relative to the amount of fucose on the same anti-WTMC construct produced in a wild-type CHO cell. That is, they are characterized by having a lower amount of fucose than they would otherwise have if produced by native CHO cells (e.g., a CHO cell that produce a native glycosylation pattern, such as, a CHO cell containing a native FUT8 gene). In some embodiments, the anti-WTMC construct is one wherein less than about 50%, 40%, 30%, 20%, 10%, or 5% of the N-linked glycans thereon comprise fucose. For example, the amount of fucose in such an anti-WTMC construct may be from 1% to 80%, from 1% to 65%, from 5% to 65% or from 20% to 40%. In some embodiments, the anti-WTMC construct is one wherein none of the N-linked glycans thereon comprise fucose, i.e., wherein the anti-WTMC construct is completely without fucose, or has no fucose or is afucosylated. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as α-1,6-fucosyltransferase gene, FUT8 knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Anti-WTMC construct (such as full-length anti-WTMC antibody) variants are further provided with bisected oligosaccharides, e.g., in which a biantennary oligosaccharide attached to the Fc region of the anti-WTMC construct is bisected by GlcNAc. Such anti-WTMC construct (such as full-length anti-WTMC antibody) variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). Anti-WTMC construct (such as full-length anti-WTMC antibody) variants with at least one galactose residue in the oligosaccharide attached to the Fc region are also provided. Such anti-WTMC construct variants may have improved CDC function. Such antibody variants are described, e.g., in WO 1997/30087 (Patel et al.); WO 1998/58964 (Raju, S.); and WO 1999/22764 (Raju, S.).

In some embodiments, the anti-WTMC construct (such as full-length anti-WTMC antibody) variants comprising an Fc region are capable of binding to an FcγRIII. In some embodiments, the anti-WTMC construct (such as full-length anti-WTMC antibody) variants comprising an Fc region have ADCC activity in the presence of human effector cells or have increased ADCC activity in the presence of human effector cells compared to the otherwise same anti-WTMC construct (such as full-length anti-WTMC antibody) comprising a human wild-type IgG1Fc region.

### Glycosylation Variants of Anti-WTMC Constructs

In some embodiments, an anti-WTMC construct provided herein is altered to increase or decrease the extent to which the anti-WTMC construct is glycosylated. Addition or deletion of glycosylation sites to an anti-WTMC construct may be conveniently accomplished by altering the amino acid sequence of the anti-WTMC construct or polypeptide portion thereof such that one or more glycosylation sites is created or removed.

Where the anti-WTMC construct comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-WTMC construct described herein may be made in order to create anti-WTMC construct glycosylation variants with certain improved properties.

In some embodiments, the anti-WTMC construct (such as a full-length anti-WTMC antibody) comprises an Fc region wherein a carbohydrate structure attached to the Fc region has reduced fucose or lacks fucose, which may improve ADCC function. In some embodiments, the anti-WTMC construct (such as full-length anti-WTMC antibody) has reduced fucose relative to the amount of fucose on the same anti-WTMC construct (e.g., the full-length anti-WTMC antibody) produced in a wild-type CHO cell (e.g., a CHO cell that produce a native glycosylation pattern, such as, a CHO cell containing a native FUT8 gene). In some embodiments, the anti-WTMC construct is one wherein less than about 50%, 40%, 30%, 20%, 10%, or 5% of the N-linked glycans thereon comprise fucose. For example, the amount of fucose in such an anti-WTMC construct may be from 1% to 80%, from 1% to 65%, from 5% to 65% or from 20% to 40%. In some embodiments, the anti-WTMC construct is one wherein none of the N-linked glycans thereon comprise fucose, i.e., wherein the anti-WTMC construct is completely without fucose, or has no fucose or is afucosylated. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as α-1,6-fucosyltransferase gene, FUT8 knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In some embodiments, the anti-WTMC construct (such as a full-length anti-WTMC antibody) is a glycosylation variant comprising bisected oligosaccharides, e.g., in which a biantennary oligosaccharide attached to the Fc region of the anti-WTMC construct is bisected by GlcNAc. Such anti-WTMC construct (e.g., a full-length anti-WTMC antibody) glycosylation variants may have reduced fucosylation and/or improved ADCC function. Examples of such antibody variants are described, e.g., in WO 2003/011878 (Jean-Mairet et al.); U.S. Pat. No. 6,602,684 (Umana et al.); US 2005/0123546 (Umana et al.), and Ferrara et al., Biotechnology and Bioengineering, 93(5): 851-861 (2006). In some embodiments, the anti-WTMC construct (such as a full-length anti-WTMC antibody) is a glycosylation variant comprising at least one galactose residue in the oligosaccharide attached to the Fc region. Such anti-WTMC construct glycosylation variants may have improved CDC function. Such glycosylation variants are described, e.g., in WO 1997/30087 (Patel et al.); WO 1998/58964 (Raju, S.); and WO 1999/22764 (Raju, S.).

In some embodiments, the anti-WTMC construct (such as full-length anti-WTMC antibody) glycosylation variant comprises an Fc region capable of binding to an FcγRIII. In some embodiments, the anti-WTMC construct (such as full-length anti-WTMC antibody) glycosylation variant comprises an Fc region have ADCC activity in the presence of human effector cells or have increased ADCC activity in the presence of human effector cells compared to an anti-WTMC construct (such as a full-length anti-WTMC antibody) comprising a human wild-type IgG1 Fc region.

### Cysteine Engineered Variants of Anti-WTMC Constructs

In some embodiments, it may be desirable to create cysteine engineered anti-WTMC constructs (such as full-length anti-WTMC antibodies) in which one or more amino acid residues are substituted with cysteine residues. In some embodiments, the substituted residues occur at accessible sites of the anti-WTMC construct. By substituting those residues with cysteine, reactive thiol groups are thereby positioned at accessible sites of the anti-WTMC construct and may be used to conjugate the anti-WTMC construct to other moieties, such as drug moieties or linker-drug moieties, to create an anti-WTMC immunoconjugate, as described further herein. Cysteine engineered anti-WTMC constructs (such as full-length anti-WTMC antibodies) may be generated as described, e.g., in U.S. Pat. No. 7,521,541.

### Derivatized Anti-WTMC Constructs

In some embodiments, the anti-WTMC construct has been further modified to contain additional nonproteinaceous moieties that are known in the art and readily available. The moieties suitable for derivatization of an anti-WTMC construct of the present disclosure include, but are not limited to, water soluble polymers. Non-limiting examples of water soluble polymers include, without limitation, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight and may be branched or unbranched. The number of polymers attached to a derivatized anti-WTMC construct may vary, and if more than one polymer is attached, the polymers can be the same or different molecules. In general, the number and/or type of polymers used for derivatization can be determined based on considerations including, but not limited to, the particular properties or functions of the anti-WTMC construct to be improved, whether the anti-WTMC construct derivative will be used in a therapy under defined conditions, etc.

In some embodiments, conjugates of an anti-WTMC construct and nonproteinaceous moiety that may be selectively heated by exposure to radiation are provided. In some embodiments, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the anti-WTMC construct-nonproteinaceous moiety are killed.

### Nucleic Acids, Vectors, and Host Cells and Preparation Thereof

Also provided are nucleic acid molecules (including sets of nucleic acid molecules) that encode the polypeptide portions of the anti-WTMC constructs described herein. In some embodiments, the nucleic acid (or a set of nucleic acids) encodes a full-length anti-WTMC antibody. In some embodiments, the nucleic acid (or a set of nucleic acids) encodes a multispecific anti-WTMC molecule (e.g., a multispecific anti-WTMC antibody, a bispecific anti-WTMC antibody, or a tandem di-scFv that comprises an anti-WTMC antibody moiety), or polypeptide portion thereof. In some embodiments, the nucleic acid (or a set of nucleic acids) encodes an anti-WTMC CAR. In some embodiments, the nucleic acid (or set of nucleic acids) encodes an anti-WTMC caTCR. In some embodiments, the two chains of the anti-WTMC caTCR are encoded on the same nucleic acid. In some embodiments, the two chains of the anti-WTMC caTCR are encoded on separate nucleic acids. In some embodiments, the nucleic acid encodes an anti-WTMC CSR. In some embodiments, the nucleic acid (or a set of nucleic acids) encodes an anti-WTMC immunoconjugate, or polypeptide portion thereof.

Nucleic acid sequence variants that encode the polypeptide portions of the anti-WTMC constructs described herein are also provided. For example, the variants include nucleotide sequences that hybridize to the nucleic acid sequences encoding an anti-WTMC construct or anti-WTMC antibody moiety described herein under at least moderately stringent hybridization conditions.

Also provided are vectors (such as expression vectors) comprising one or more nucleic acids described herein.

An anti-WTMC construct described herein, or polypeptide portion thereof, e.g., an anti-WTMC CAR can be expressed from a natural or synthetic nucleic acid encoding the anti-WTMC construct or polypeptide portion thereof. Briefly, the nucleic acid may be inserted into an appropriate expression vector, such that the nucleic acid is operably linked to 5′ and 3′ regulatory elements, including for example a promoter (e.g., a lymphocyte-specific promoter) and a 3′ untranslated region (UTR). The vectors are preferable suitable for replication and integration in eukaryotic host cells. Typical cloning and expression vectors contain transcription and translation terminators, initiation sequences, and promoters useful for regulation of the expression of the desired nucleic acid sequence.

The nucleic acids described herein may also be used for nucleic acid immunization and gene therapy, using standard gene delivery protocols. Methods for gene delivery are known in the art. See, e.g., U.S. Pat. Nos. 5,399,346; 5,580,859; and 5,589,466; which are incorporated by reference herein in their entireties. In some embodiments, there is provided a gene therapy vector.

The nucleic acids described herein may be cloned into any of a variety of vectors known in the art. For example, the nucleic acid (or set of nucleic acids) can be cloned into, e.g., a plasmid, a phagemid, a phage derivative, an animal virus, and/or a cosmid. Vectors of particular interest include expression vectors, replication vectors, probe generation vectors, and sequencing vectors.

In some embodiments, the expression vector comprising a nucleic acid encoding an anti-WTMC construct or anti-WTMC antibody moiety described herein is a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, the viral vector is an adenovirus vector. A number of adenovirus vectors are known in the art. In some embodiments, the viral vector is a lentivirus vector. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia virus (MLV), Abelson murine leukemia virus (A-MLV), ecotropic murine leukemia virus (EcoMLV), in that they can transduce non-proliferating cells. They also have the added advantage of low immunogenicity.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukosis virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the expression of anti-WTMC constructs described herein should not be limited to the use of constitutive promoters. Inducible promoters are also contemplated. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

In order to assess the expression of a polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Exemplary selectable markers include, but are not limited to, e.g., antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, β-galactosidase, chloramphenicol acetyl transferase, secreted alkaline phosphatase, or the green fluorescent protein gene (e.g., Ui-Tel et al., 2000 FEBS Letters 479: 79-82). Suitable expression systems are well known and may be prepared using known techniques or obtained commercially. In general, the anti-WTMC construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). In some embodiments, the introduction of a polynucleotide into a host cell is carried out by calcium phosphate transfection.

Biological methods for introducing a polynucleotide of interest into a host cell include the use of DNA and RNA vectors. Viral vectors, and especially retroviral vectors, have become the most widely used method of inserting genes into mammalian, e.g., human cells. Other viral vectors can be derived from lentivirus, poxviruses, herpes simplex virus 1, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Another exemplary delivery vehicle is a liposome. The use of lipid formulations is contemplated for the introduction of nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the present disclosure.

### Pharmaceutical Compositions

Also provided herein are compositions (such as pharmaceutical compositions, also referred to herein as formulations) comprising an anti-WTMC construct or a nucleic acid or a vector encoding an anti-WTMC construct. In some embodiments, the composition further comprises a cell (such as an effector cell, e.g., a T cell) associated with (including but not limited to comprising or expressing) the anti-WTMC construct. In some embodiments, there is provided a pharmaceutical composition comprising an anti-WTMC construct, or a nucleic acid or a vector encoding an anti-WTMC construct, and a pharmaceutically acceptable carrier. In some embodiments, the pharmaceutical composition further comprises a cell (such as an effector cell, e.g., a T cell) associated with (including but not limited to comprising or expressing) the anti-WTMC construct.

Suitable formulations of the anti-WTMC constructs are obtained by mixing an anti-WTMC construct having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as olyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). Exemplary formulations are described in WO98/56418, expressly incorporated herein by reference. Lyophilized formulations adapted for subcutaneous administration are described in WO97/04801. Such lyophilized formulations may be reconstituted with a suitable diluent to a high protein concentration and the reconstituted formulation may be administered subcutaneously to the individual to be treated herein. Lipofectins or liposomes can be used to deliver the anti-WTMC constructs of this present application into cells.

The formulation herein may also contain one or more active compounds in addition to the anti-WTMC construct as necessary for the particular indication being treated, preferably those with complementary activities that do not adversely affect each other. For example, it may be desirable to further provide an anti-neoplastic agent, a growth inhibitory agent, a cytotoxic agent, or a chemotherapeutic agent in addition to the anti-WTMC construct. Such molecules are suitably present in combination in amounts that are effective for the purpose intended. The effective amount of such other agents depends on the amount of anti-WTMC construct present in the formulation, the type of disease or disorder or treatment, and other factors discussed above. These are generally used in the same dosages and with administration routes as described herein or about from 1 to 99% of the heretofore employed dosages.

The anti-WTMC constructs may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980). Sustained-release preparations may be prepared.

Sustained-release preparations of the anti-WTMC constructs can be prepared. Suitable examples of sustained-release preparations include semipermeable matrices of solid hydrophobic polymers containing the antibody (or fragment thereof), which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate, AbbVie Inc., North Chicago, IL), and poly-D-(−)-3-hydroxybutyric acid. While polymers such as ethylene-vinyl acetate and lactic acid-glycolic acid enable release of molecules for over 100 days, certain hydro gels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization of anti-WTMC constructs depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

In some embodiments, the anti-WTMC construct is formulated in a buffer comprising a citrate, NaCl, acetate, succinate, glycine, polysorbate 80 (Tween 80), or any combination of the foregoing. In some embodiments, the anti-WTMC construct is formulated in a buffer comprising about 100 mM to about 150 mM glycine. In some embodiments, the anti-WTMC construct is formulated in a buffer comprising about 50 mM to about 100 mM NaCl. In some embodiments, the anti-WTMC construct is formulated in a buffer comprising about 10 mM to about 50 mM acetate. In some embodiments, the anti-WTMC construct is formulated in a buffer comprising about 10 mM to about 50 mM succinate. In some embodiments, the anti-WTMC construct is formulated in a buffer comprising about 0.005% to about 0.02% polysorbate 80. In some embodiments, the anti-WTMC construct is formulated in a buffer having a pH between about 5.1 and 5.6. In some embodiments, the anti-WTMC construct is formulated in a buffer comprising 10 mM citrate, 100 mM NaCl, 100 mM glycine, and 0.01% polysorbate 80, wherein the formulation is at pH 5.5.

The formulations to be used for in vivo administration must be sterile. This is readily accomplished by, e.g., filtration through sterile filtration membranes.

### Methods for Treatment Using Anti-WTMC Constructs

The anti-WTMC constructs and/or compositions of the present application can be administered to individuals (e.g., mammals such as humans) to treat a Wilm's Tumor 1 (WT1)-related disease, including, for example, a disease characterized by aberrant expression of WT1 (e.g., WT1-RMF) or expression of a mutant WT1 (such as cancer). Examples of WT1-related disease include, without limitation, cancer, e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma). The present application thus in some embodiments provides a method of treating a WT1-related disease in an individual comprising administering to the individual an effective amount of a composition (such as a pharmaceutical composition) comprising an anti-WTMC construct comprising an anti-WTMC antibody moiety, such as any one of the anti-WTMC constructs described herein. In some embodiments, the composition further comprises a cell (such as an effector cell) associated with (including but not limited to comprising or expressing) the anti-WTMC construct. In some embodiments, the disease is cancer. In some embodiments, the cancer is a WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma).

Thus, provided herein is a method of treating a WT1-associated disease (such as cancer) in an individual, which method comprises administering to the individual an effective amount of a composition (such as a pharmaceutical composition) comprising an anti-WTMC construct or construct combination described herein. In some embodiments, the composition further comprises a cell (such as an effector cell) associated with (including but not limited to comprising or expressing) the anti-WTMC construct or construct combination (such as an effector cell that expresses an anti-WTMC construct or construct combination described herein). In some embodiments, the cancer is a WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma). In some embodiments, the individual is human.

In some embodiments, the anti-WTMC construct or construct combination used in the method is non-naturally occurring. In some embodiments, the anti-WTMC construct used in the method is a full-length antibody, a multispecific (such as bispecific) anti-WTMC construct, an anti-WTMC chimeric antigen receptor (CAR), an anti-WTMC chimeric antibody-T cell receptor construct (caTCR), an anti-WTMC chimeric signaling receptors (CSRs), an anti-WTMC immunoconjugate, or any other anti-WTMC construct described in further detail elsewhere herein. In some embodiments, an anti-WTMC construct combination comprising anti-WTMC CSR and a CAR (e.g., an anti-WTMC CSR+CAR construct combination described elsewhere herein) is used in the method. In some embodiments, the CAR binds to a target ligand other than a WTMC complex. In some embodiments, an anti-WTMC construct combination comprising anti-WTMC CSR and a caTCR (e.g., an anti-WTMC CSR+caTCR construct combination described elsewhere herein) is used in the method. In some embodiments, the binds to a target ligand other than a WTMC complex. In some embodiments, an anti-WTMC construct combination comprising anti-WTMC CAR and a CSR (e.g., an anti-WTMC CAR+CSR construct combination described elsewhere herein) is used in the method. In some embodiments, the CSR binds to a target ligand other than a WTMC complex. In some embodiments, an anti-WTMC construct combination comprising anti-WTMC CAR and an anti-WTMC CSR (e.g., an anti-WTMC CAR+anti-WTMC CSR construct combination described elsewhere herein) is used in the method. In some embodiments, an anti-WTMC construct combination comprising anti-WTMC caTCR and a CSR (e.g., an anti-WTMC caTCR+CSR construct combination described elsewhere herein) is used in the method. In some embodiments, the CSR binds to a target ligand other than a WTMC complex. In some embodiments, an anti-WTMC construct combination comprising anti-WTMC caTCR and an anti-WTMC CSR (e.g., an anti-WTMC caTCR+anti-WTMC CSR construct combination described elsewhere herein) is used in the method. Each of the anti-WTMC constructs or construct combinations described herein demonstrates high specificity for human WTMC complex in native form. In some embodiments, the pharmaceutical composition used in the method further comprises a cell (such as an effector cell) that expresses or is associated with the anti-WTMC construct or construct combination. In some embodiments, the WT1-associated disease is cancer. In some embodiments, the cancer is, for example, a WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma). In some embodiments, the individual is human.

In some embodiments of any of the methods for treating a WT1-associated disease described herein, the anti-WTMC construct or construct combination is conjugated to a cell (such as an immune cell, e.g., a T cell) prior to being administered to the individual. Thus, provided is a method of treating a WT1-associated disease in an individual comprising a) conjugating any one of the anti-WTMC constructs or construct combinations described herein to a cell (such as an immune cell, e.g., a T cell) to form an anti-WTMC construct/cell conjugate, and b) administering an effective amount of a composition comprising the anti-WTMC construct/cell conjugate to the individual. In some embodiments, the cell to which the anti-WTMC construct or construct combination is conjugated is derived from the individual being treated. In some embodiments, the cell to which the anti-WTMC construct or construct combination is conjugated is not derived from the individual being treated. In some embodiments, the anti-WTMC construct or construct combination is conjugated to the cell by covalent linkage to a molecule on the surface of the cell. In some embodiments, the anti-WTMC construct or construct combination is conjugated to the cell by non-covalent linkage to a molecule on the surface of the cell. In some embodiments, the anti-WTMC construct or construct combination is conjugated to the cell by insertion of a portion of the anti-WTMC construct or construct combination into the outer membrane of the cell. In some embodiments, the anti-WTMC construct or construct combination is non-naturally occurring. In some embodiments, the anti-WTMC construct used in the method is a full-length antibody, a multispecific (such as bispecific) anti-WTMC construct (such as a tandem di-scFv), an anti-WTMC immunoconjugate, or any other anti-WTMC construct described in further detail elsewhere herein. In some embodiments, an anti-WTMC construct combination comprising anti-WTMC CSR and a CAR (e.g., an anti-WTMC CSR+CAR construct combination described elsewhere herein) is used in the method. In some embodiments, the CAR binds to a target ligand other than a WTMC complex. In some embodiments, an anti-WTMC construct combination comprising anti-WTMC CSR and a caTCR (e.g., an anti-WTMC CSR+caTCR construct combination described elsewhere herein) is used in the method. In some embodiments, the caTCR binds to a target ligand other than a WTMC complex. In some embodiments, an anti-WTMC construct combination comprising anti-WTMC CAR and a CSR (e.g., an anti-WTMC CAR+CSR construct combination described elsewhere herein) is used in the method. In some embodiments, the CSR binds to a target ligand other than a WTMC complex. In some embodiments, an anti-WTMC construct combination comprising anti-WTMC CAR and an anti-WTMC CSR (e.g., an anti-WTMC CAR+anti-WTMC CSR construct combination described elsewhere herein) is used in the method. In some embodiments, an anti-WTMC construct combination comprising anti-WTMC caTCR and a CSR (e.g., an anti-WTMC caTCR+a CSR construct combination described elsewhere herein) is used in the method. In some embodiments, the CSR binds to a target ligand other than a WTMC complex. In some embodiments, an anti-WTMC construct combination comprising anti-WTMC caTCR and an anti-WTMC CSR (e.g., an anti-WTMC caTCR+anti-WTMC CSR construct combination described elsewhere herein) is used in the method. In some embodiments, the WT1-associated disease is cancer. In some embodiments, the cancer is a WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma). In some embodiments, the individual is human.

In some embodiments of any of the methods for treating a WT1-associated disease described herein, treatment comprises administering to a recipient in need a cell (e.g., a T cell, such as an αβ T cell, a γδ T cell, a cytotoxic T cell, a helper T cell, or a natural killer T cell) that has been genetically modified (i.e., transduced or transfected, such as in vitro) with a nucleic acid, a set of nucleic acids, a vector, or a set of vectors encoding an anti-WTMC CAR, anti-WTMC caTCR, anti-WTMC tandem multispecific scFv (such as a tandem di-scFv), anti-WTMC CSR, an anti-WTMC CSR+CAR construct combination, an anti-WTMC CSR+caTCR construct combination, an anti-WTMC CAR+CSR construct combination, an anti-WTMC CAR+anti-WTMC CSR construct combination, anti-WTMC caTCR+CSR construct combination, or an anti-WTMC caTCR+anti-WTMC CSR construct combination disclosed herein. In some embodiments, the genetically modified cell (e.g., a T cell, such as an αβ T cell, a γδ T cell, a cytotoxic T cell, a helper T cell, a natural killer T cell, and a suppressor T cell) expresses the anti-WTMC CAR, anti-WTMC caTCR, anti-WTMC tandem multispecific scFv (such as a tandem di-scFv), anti-WTMC CSR, an anti-WTMC CSR+CAR construct combination, an anti-WTMC CSR+caTCR construct combination, an anti-WTMC CAR+CSR construct combination, an anti-WTMC CAR+anti-WTMC CSR construct combination, anti-WTMC caTCR+CSR construct combination, or an anti-WTMC caTCR+anti-WTMC CSR construct combination encoded by the nucleic acid, set of nucleic acids, vector, or set of vectors. In some embodiments, the recipient is a mammal, such as a human, e.g., a human who has or is suspected of having the WT1-associated disease).

In some embodiments of the methods for treating a WT1-associated disease, treatment further comprises the step of genetically modifying (i.e., transducing or transfecting, such as in vitro) the cell (e.g., a T cell, such as an αβ T cell, a γδ T cell, a cytotoxic T cell, a helper T cell, or a natural killer T cell) with the nucleic acid, the set of nucleic acids, the vector, or the set of vectors encoding the anti-WTMC CAR, anti-WTMC caTCR, anti-WTMC tandem multispecific scFv (such as a tandem di-scFv), or anti-WTMC CSR prior to administration to the recipient. In some embodiments, the cell (e.g., a T cell, such as an αβ T cell, a γδ T cell, a cytotoxic T cell, a helper T cell, or a natural killer T cell) has been genetically modified (i.e., transduced or transfected, such as in vitro) with the nucleic acid, the set of nucleic acids, the vector, or the set of vectors encoding the anti-WTMC CAR, and is further genetically modified (i.e., transduced or transfected, such as in vitro) with a nucleic acid, a set of nucleic acids, a vector, or a set of vectors encoding a multispecific scFv (such as a tandem di-scFv) or a CSR. In some embodiments, the cell (e.g., a T cell, such as an αβ T cell, a γδ T cell, a cytotoxic T cell, a helper T cell, or a natural killer T cell) has been genetically modified (i.e., transduced or transfected, such as in vitro) with the nucleic acid, the set of nucleic acids, the vector, or the set of vectors encoding the anti-WTMC caTCR, and is further genetically modified (i.e., transduced or transfected, such as in vitro) with a nucleic acid, a set of nucleic acids, a vector, or a set of vectors encoding a multispecific scFv (such as a tandem di-scFv) or a CSR. In some embodiments, the multispecific scFv (e.g., a tandem di-scFv) targets a WTMC complex. In some embodiments, the multispecific scFv (e.g., a tandem di-scFv) targets a different antigen (e.g., an antigen other than WT1). In some embodiments, the second scFv binds to a target ligand other than a WTMC complex. In some embodiments, the CSR targets a WTMC complex. In some embodiments, the CSR binds to a target ligand other than a WTMC complex. In some embodiments, the cell (e.g., a T cell, such as an αβ T cell, a γδ T cell, a cytotoxic T cell, a helper T cell, or a natural killer T cell) has been genetically modified (i.e., transduced or transfected, such as in vitro) with the nucleic acid, the set of nucleic acids, the vector, or the set of vectors encoding the anti-WTMC tandem multispecific scFv (such as a tandem di-scFv) or the anti-WTMC CSR, and is further genetically modified (i.e., transduced or transfected, such as in vitro) with a nucleic acid, a set of nucleic acids, a vector, or a set of vectors encoding a CAR or caTCR. In some embodiments, the CAR or caTCR targets a WTMC complex. In some embodiments, the CAR or caTCR binds to a target ligand other than a WTMC complex. In some embodiments, the anti-WTMC construct combination comprises an anti-WTMC CAR and anti-WTMC CSR (e.g., an anti-WTMC CAR+anti-WTMC CSR construct combination described elsewhere herein). In some embodiments, the anti-WTMC construct combination comprises an anti-WTMC caTCR and anti-WTMC CSR (e.g., an anti-WTMC caTCR+anti-WTMC CSR construct combination described elsewhere herein).

In some embodiments of the methods for treating a WT1-associated disease, treatment further comprises the step of obtaining (such as isolating) cells (e.g., T cells, such as αβ T cells, a γδ T cells, cytotoxic T cells, helper T cells, or natural killer T cells) from an individual (e.g., a mammal, such as a human, e.g., a human who has or is suspected of having the WT1-associated disease) prior to the step of genetically modifying (i.e., transducing or transfecting, such as in vitro) the cells with a nucleic acid, a set of nucleic acids, a vector, or a set of vectors encoding the anti-WTMC CAR, anti-WTMC caTCR, anti-WTMC tandem multispecific scFv (such as a tandem di-scFv), anti-WTMC CSR, an anti-WTMC CSR+CAR construct combination, an anti-WTMC CSR+caTCR construct combination, an anti-WTMC CAR+CSR construct combination, an anti-WTMC CAR+anti-WTMC CSR construct combination, anti-WTMC caTCR+CSR construct combination, or anti-WTMC caTCR+anti-WTMC CSR construct combination, e.g., as described above. In some embodiments, the recipient to whom the genetically modified cells are administered is the individual from whom the cells were obtained. Such a genetically modified immune cell is referred to as an “autologous anti-WTMC effector cell.” In some embodiments, the recipient to whom the genetically modified cells are administered is not the individual from whom the cells were obtained. Such a genetically modified immune cell is referred to as a “heterologous anti-WTMC effector cell.” In some embodiments, the heterologous anti-WTMC cell is allogeneic, syngeneic, or xenogeneic with respect to the recipient.

In some embodiments, the individual is a mammal (e.g., a human, a non-human primate (such as a rhesus monkey or a cynomolgus monkey), a rat, a mouse, a cow, a horse, a pig, a sheep, a goat, a dog, a cat, etc.). In some embodiments, the individual is a human. In some embodiments, the individual is a clinical patient, a clinical trial volunteer, an experimental animal, etc. In some embodiments, the individual is younger than about 60 years old (including for example younger than about any of 50, 40, 30, 25, 20, 15, or 10 years old). In some embodiments, the individual is older than about 60 years old (including for example older than about any one of 70, 75, 80, 85, 90, 95, 100, or more than 100 years old). In some embodiments, the individual is diagnosed with or genetically prone to one or more of the WT1-associated diseases or disorders described herein (e.g., a WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)). In some embodiments, the individual has one or more risk factors associated with one or more WT1-associated diseases or disorders described herein.

Also provided is a method of delivering an anti-WTMC construct (such as any one of the anti-WTMC constructs described herein) or an anti-WTMC construct combination (such as any one of the anti-WTMC construct combinations described herein) to a cell expressing the WTMC complex, the method comprising administering to the individual a composition comprising the anti-WTMC construct or construct combination. In some embodiments, the anti-WTMC construct or construct combination to be delivered is associated with a cell (such as an effector cell, e.g., a T cell).

Many diagnostic methods for WT1-associated cancer e.g., chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma or any other WT1-associated disease, e.g., a disease exhibiting WT1 expression and the clinical delineation of those diseases are known in the art. Such methods include, but are not limited to, e.g., immunohistochemistry, PCR, and fluorescent in situ hybridization (FISH).

In some embodiments, the anti-WTMC constructs, anti-WTMC construct combinations, and/or compositions of the present disclosure are administered in combination with a second, third, or fourth agent (including, e.g., an antineoplastic agent, a growth inhibitory agent, a cytotoxic agent, or a chemotherapeutic agent) to treat WT1-associated diseases or disorders, e.g., diseases involving WT1 expression. In some embodiments, the anti-WTMC construct or construct combination is administered in combination with an agent that increases the expression of WT1 on diseased cells (such as cancer cells). In some embodiments, the agent is a chemotherapeutic agent including, for example, topotecan, etoposide, cisplatin, paclitaxel, and vinblastine.

The efficacy of cancer treatments can be evaluated, for example, by a variety of well-known methods including, without limitation, e.g., tumor regression, tumor weight or size shrinkage, time to progression, duration of survival, progression free survival, overall response rate, duration of response, quality of life, WT1 protein expression and/or WT1 activity. Approaches to determining efficacy of the therapy can be employed, including for example, measurement of response through radiological imaging.

In some embodiments, the efficacy of a method of treatment is measured as the percentage tumor growth inhibition (% TGI), calculated using the equation (1−{Tₜ/T₀/Cₜ/C₀}/1−{C₀/Cₜ})×100), where T is the mean relative tumor volume of the treated tumor, and C is the mean relative tumor volume of a non-treated tumor (at time t or time 0; Tₜ or To or Ct or Co, respectively). In some embodiments, the % TGI is about any one of 10%, 20%, 30%, 40%, 50%, 60%, 70%, 80%, 90%, 91%, 92%, 93%, 94%, 95%, or more than 95% (e.g., up to 100%), including any range in between these values.

For example, in some embodiments, there is provided a method of treating a WT1-associated disease (such as cancer, e.g., a WT1-positive cancer (such as chronic myelocytic leukemia, multiple myeloma (MM), acute lymphoblastic leukemia (ALL), acute myeloid/myelogenous leukemia (AML), myelodysplastic syndrome (MD), mesothelioma, ovarian cancer, gastrointestinal cancers, breast cancer, prostate cancer or glioblastoma)) in an individual comprising administering to the individual an effective amount of a composition comprising an anti-WTMC construct comprising any one of the anti-WTMC antibody moieties described herein that specifically binds to a complex comprising a WT1 peptide (e.g., WT1-RMF) and an MHC class I protein. In some embodiments, the WT1 peptide comprises (such as consists of) the amino acid sequence of SEQ ID NO: 113. In some embodiments, the antibody moiety specifically recognizes amino acid residues FPNA (SEQ ID NO: 349) of SEQ ID NO: 113. In some embodiments, the MHC class I protein is HLA-A02. In some embodiments, the MHC class I protein is HLA-A*02:01. In some embodiments, the anti-WTMC construct is non-naturally occurring. In some embodiments, the anti-WTMC construct is a full-length antibody. In some embodiments, the anti-WTMC construct is a multi-specific (such as bispecific) molecule. In some embodiments, the anti-WTMC construct is a chimeric receptor (e.g., a caTCR, CSR, or a combination thereof). In some embodiments, the anti-WTMC construct is an immunoconjugate. In some embodiments, the composition further comprises a cell (such as an effector cell) associated with (including but not limited to comprising or expressing) the anti-WTMC construct. In some embodiments, the individual is human. In some embodiments, the WT1-related disease is a cancer. In some embodiments, the cancer is a WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma).

In some embodiments, there is provided a method of treating a WT1-associated disease (such as cancer, e.g., a WT1-positive cancer (such as chronic myelocytic leukemia, multiple myeloma (MM), acute lymphoblastic leukemia (ALL), acute myeloid/myelogenous leukemia (AML), myelodysplastic syndrome (MD), mesothelioma, ovarian cancer, gastrointestinal cancers, breast cancer, prostate cancer or glioblastoma) in an individual comprising administering to the individual an effective amount of a composition comprising any one of the anti-WTMC constructs described herein.

In some embodiments, the anti-WTMC construct is non-naturally occurring. In some embodiments, the anti-WTMC construct is a full-length antibody. In some embodiments, the anti-WTMC construct is a multi-specific (such as bispecific) molecule. In some embodiments, the anti-WTMC construct is a chimeric receptor (e.g., a CAR, caTCR, CSR, or a combination thereof). In some embodiments, the anti-WTMC construct is an immunoconjugate. In some embodiments, the composition further comprises a cell (such as an effector cell) associated with (including but not limited to comprising or expressing) the anti-WTMC construct.

In some embodiments, the anti-WTMC construct comprises an anti-WTMC antibody moiety comprising a HC-CDR1 comprising an amino acid sequence set forth in SEQ ID NO: 1, a HC-CDR2 comprising an amino acid sequence set forth in SEQ ID NO: 2, a HC-CDR3 comprising an amino acid sequence set forth in SEQ ID NO: 3, a LC-CDR1 comprising an amino acid sequence set forth in SEQ ID NO: 4, a LC-CDR2 comprising an amino acid sequence set forth in SEQ ID NO: 5, and a LC-CDR3 comprising an amino acid sequence set forth in SEQ ID NO: 6.

In some embodiments, the anti-WTMC construct comprises an anti-WTMC antibody moiety comprising a HC-CDR1 comprising an amino acid sequence set forth in SEQ ID NO: 7, a HC-CDR2 comprising an amino acid sequence set forth in SEQ ID NO: 8, a HC-CDR3 comprising an amino acid sequence set forth in SEQ ID NO: 9, a LC-CDR1 comprising an amino acid sequence set forth in SEQ ID NO: 10, a LC-CDR2 comprising an amino acid sequence set forth in SEQ ID NO: 11, and a LC-CDR3 comprising an amino acid sequence set forth in SEQ ID NO: 12.

In some embodiments, the anti-WTMC construct comprises an anti-WTMC antibody moiety comprising a HC-CDR1 comprising an amino acid sequence set forth in SEQ ID NO: 13, a HC-CDR2 comprising an amino acid sequence set forth in SEQ ID NO: 14, a HC-CDR3 comprising an amino acid sequence set forth in SEQ ID NO: 15, a LC-CDR1 comprising an amino acid sequence set forth in SEQ ID NO: 16, a LC-CDR2 comprising an amino acid sequence set forth in SEQ ID NO: 17, and a LC-CDR3 comprising an amino acid sequence set forth in SEQ ID NO: 18.

In some embodiments, the anti-WTMC construct comprises an anti-WTMC antibody moiety comprising a HC-CDR1 comprising an amino acid sequence set forth in SEQ ID NO: 19, a HC-CDR2 comprising an amino acid sequence set forth in SEQ ID NO: 20, a HC-CDR3 comprising an amino acid sequence set forth in SEQ ID NO: 21, a LC-CDR1 comprising an amino acid sequence set forth in SEQ ID NO: 22, a LC-CDR2 comprising an amino acid sequence set forth in SEQ ID NO: 23, and a LC-CDR3 comprising an amino acid sequence set forth in SEQ ID NO: 24.

In some embodiments, the anti-WTMC construct comprises an anti-WTMC antibody moiety comprising a HC-CDR1 comprising an amino acid sequence set forth in SEQ ID NO: 25, a HC-CDR2 comprising an amino acid sequence set forth in SEQ ID NO: 26, a HC-CDR3 comprising an amino acid sequence set forth in SEQ ID NO: 27, a LC-CDR1 comprising an amino acid sequence set forth in SEQ ID NO: 28, a LC-CDR2 comprising an amino acid sequence set forth in SEQ ID NO: 29, and a LC-CDR3 comprising an amino acid sequence set forth in SEQ ID NO: 30.

In some embodiments, the anti-WTMC construct comprises an anti-WTMC antibody moiety comprising a HC-CDR1 comprising an amino acid sequence set forth in SEQ ID NO: 31, a HC-CDR2 comprising an amino acid sequence set forth in SEQ ID NO: 32, a HC-CDR3 comprising an amino acid sequence set forth in SEQ ID NO: 33, a LC-CDR1 comprising an amino acid sequence set forth in SEQ ID NO: 34, a LC-CDR2 comprising an amino acid sequence set forth in SEQ ID NO: 35, and a LC-CDR3 comprising an amino acid sequence set forth in SEQ ID NO: 36.

In some embodiments, the anti-WTMC construct comprises an anti-WTMC antibody moiety comprising a HC-CDR1 comprising an amino acid sequence set forth in SEQ ID NO: 37, a HC-CDR2 comprising an amino acid sequence set forth in SEQ ID NO: 38, a HC-CDR3 comprising an amino acid sequence set forth in SEQ ID NO: 39, a LC-CDR1 comprising an amino acid sequence set forth in SEQ ID NO: 40, a LC-CDR2 comprising an amino acid sequence set forth in SEQ ID NO: 41, and a LC-CDR3 comprising an amino acid sequence set forth in SEQ ID NO: 42.

In some embodiments, the anti-WTMC construct comprises an anti-WTMC antibody moiety comprising a HC-CDR1 comprising an amino acid sequence set forth in SEQ ID NO: 43, a HC-CDR2 comprising an amino acid sequence set forth in SEQ ID NO: 44, a HC-CDR3 comprising an amino acid sequence set forth in SEQ ID NO: 45, a LC-CDR1 comprising an amino acid sequence set forth in SEQ ID NO: 46, a LC-CDR2 comprising an amino acid sequence set forth in SEQ ID NO: 47, and a LC-CDR3 comprising an amino acid sequence set forth in SEQ ID NO: 48.

In some embodiments, the anti-WTMC construct comprises an anti-WTMC antibody moiety comprising a HC-CDR1 comprising an amino acid sequence set forth in SEQ ID NO: 49, a HC-CDR2 comprising an amino acid sequence set forth in SEQ ID NO: 50, a HC-CDR3 comprising an amino acid sequence set forth in SEQ ID NO: 51, a LC-CDR1 comprising an amino acid sequence set forth in SEQ ID NO: 52, a LC-CDR2 comprising an amino acid sequence set forth in SEQ ID NO: 53, and a LC-CDR3 comprising an amino acid sequence set forth in SEQ ID NO: 54.

In some embodiments, the anti-WTMC construct comprises an anti-WTMC antibody moiety comprising a HC-CDR1 comprising an amino acid sequence set forth in SEQ ID NO: 55, a HC-CDR2 comprising an amino acid sequence set forth in SEQ ID NO: 56, a HC-CDR3 comprising an amino acid sequence set forth in SEQ ID NO: 57, a LC-CDR1 comprising an amino acid sequence set forth in SEQ ID NO: 58, a LC-CDR2 comprising an amino acid sequence set forth in SEQ ID NO: 59, and a LC-CDR3 comprising an amino acid sequence set forth in SEQ ID NO: 60.

In some embodiments, the anti-WTMC construct comprises an anti-WTMC antibody moiety comprising a HC-CDR1 comprising an amino acid sequence set forth in SEQ ID NO: 61, a HC-CDR2 comprising an amino acid sequence set forth in SEQ ID NO: 62, a HC-CDR3 comprising an amino acid sequence set forth in SEQ ID NO: 63, a LC-CDR1 comprising an amino acid sequence set forth in SEQ ID NO: 64, a LC-CDR2 comprising an amino acid sequence set forth in SEQ ID NO: 65, and a LC-CDR3 comprising an amino acid sequence set forth in SEQ ID NO: 66.

In some embodiments, the anti-WTMC construct comprises an anti-WTMC antibody moiety comprising a HC-CDR1 comprising an amino acid sequence set forth in SEQ ID NO: 67, a HC-CDR2 comprising an amino acid sequence set forth in SEQ ID NO: 68, a HC-CDR3 comprising an amino acid sequence set forth in SEQ ID NO: 69, a LC-CDR1 comprising an amino acid sequence set forth in SEQ ID NO: 70, a LC-CDR2 comprising an amino acid sequence set forth in SEQ ID NO: 71, and a LC-CDR3 comprising an amino acid sequence set forth in SEQ ID NO: 72.

In some embodiments, the anti-WTMC construct comprises an anti-WTMC antibody moiety comprising a HC-CDR1 comprising an amino acid sequence set forth in SEQ ID NO: 73, a HC-CDR2 comprising an amino acid sequence set forth in SEQ ID NO: 74, a HC-CDR3 comprising an amino acid sequence set forth in SEQ ID NO: 75, a LC-CDR1 comprising an amino acid sequence set forth in SEQ ID NO: 76, a LC-CDR2 comprising an amino acid sequence set forth in SEQ ID NO: 77, and a LC-CDR3 comprising an amino acid sequence set forth in SEQ ID NO: 78.

In some embodiments, the anti-WTMC construct comprises an anti-WTMC antibody moiety comprising a HC-CDR1 comprising an amino acid sequence set forth in SEQ ID NO: 79, a HC-CDR2 comprising an amino acid sequence set forth in SEQ ID NO: 80, a HC-CDR3 comprising an amino acid sequence set forth in SEQ ID NO: 81, a LC-CDR1 comprising an amino acid sequence set forth in SEQ ID NO: 82, a LC-CDR2 comprising an amino acid sequence set forth in SEQ ID NO: 83, and a LC-CDR3 comprising an amino acid sequence set forth in SEQ ID NO: 84.

In some embodiments, the CDRs are human CDRs. In some embodiments, the anti-WTMC antibody moiety comprising one, two, or three CDRs of a V_{H} domain comprising any one of SEQ ID NOs: 85, 87, 89, 91, 93, 95, 97, 99, 101, 103, 105, 107, 109 or 111 and one, two, or three CDRs of a V_{L} domain comprising any one of SEQ ID NOs: 86, 88, 90, 92, 94, 96, 98, 100, 102, 104, 106, 108, 110 or 112.

In some embodiments, the anti-WTMC antibody moiety comprising a V_{H} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to any one of SEQ ID NOs: 85, 87, 89, 91, 93, 95, 97, 99, 101, 103, 105, 107, 109 or 111 and/or a V_{L} domain comprising the amino acid sequence that is at least about 85% (e.g., at least about any one of 85%, 86%, 87%, 88%, 89%, 90%, 91%, 92%, 93%, 94%, 95%, 96%, 97%, 98%, 99% or 100%) identical to any one of SEQ ID NOs: 86, 88, 90, 92, 94, 96, 98, 100, 102, 104, 106, 108, 110 or 112.

In some embodiments, the anti-WTMC antibody moiety comprising a V_{H} domain comprising SEQ ID NO: 85 and a V_{L} domain comprising SEQ ID NO: 86.

In some embodiments, the anti-WTMC antibody moiety comprising a V_{H} domain comprising SEQ ID NO: 87 and a V_{L} domain comprising SEQ ID NO: 88.

In some embodiments, the anti-WTMC antibody moiety comprising a V_{H} domain comprising SEQ ID NO: 89 and a V_{L} domain comprising SEQ ID NO: 90.

In some embodiments, the anti-WTMC antibody moiety comprising a V_{H} domain comprising SEQ ID NO: 91 and a V_{L} domain comprising SEQ ID NO: 92.

In some embodiments, the anti-WTMC antibody moiety comprising a V_{H} domain comprising SEQ ID NO: 93 and a V_{L} domain comprising SEQ ID NO: 94.

In some embodiments, the anti-WTMC antibody moiety comprising a V_{H} domain comprising SEQ ID NO: 95 and a V_{L} domain comprising SEQ ID NO: 96.

In some embodiments, the anti-WTMC antibody moiety comprising a V_{H} domain comprising SEQ ID NO: 97 and a V_{L} domain comprising SEQ ID NO: 98.

In some embodiments, the anti-WTMC antibody moiety comprising a V_{H} domain comprising SEQ ID NO: 99 and a V_{L} domain comprising SEQ ID NO: 100.

In some embodiments, the anti-WTMC antibody moiety comprising a V_{H} domain comprising SEQ ID NO: 101 and a V_{L} domain comprising SEQ ID NO: 102.

In some embodiments, the anti-WTMC antibody moiety comprising a V_{H} domain comprising SEQ ID NO: 103 and a V_{L} domain comprising SEQ ID NO: 104.

In some embodiments, the anti-WTMC antibody moiety comprising a V_{H} domain comprising SEQ ID NO: 105 and a V_{L} domain comprising SEQ ID NO: 106.

In some embodiments, the anti-WTMC antibody moiety comprising a V_{H} domain comprising SEQ ID NO: 107 and a V_{L} domain comprising SEQ ID NO: 108.

In some embodiments, the anti-WTMC antibody moiety comprising a V_{H} domain comprising SEQ ID NO: 109 and a V_{L} domain comprising SEQ ID NO: 110.

In some embodiments, the anti-WTMC antibody moiety comprising a V_{H} domain comprising SEQ ID NO: 111 and a V_{L} domain comprising SEQ ID NO: 112.

### Dosing and Method of Administering the Anti-WTMC Construct Compositions

The dose of a pharmaceutical composition comprising DNA or protein or a T cell expressing an anti-WTMC construct or construct combination described herein that is administered to an individual (such as a human) may vary with the particular composition, the mode of administration, and the type of disease being treated. In some embodiments, the amount of the pharmaceutical composition is effective to result in an objective response (e.g., in the case of solid tumor, a partial response (PR) or a complete response (CR), e.g., according to RECIST criteria (Response Evaluation Criteria in Solid Tumors) described in Eisenhauer et al. (2009) European Journal of Cancer, 45 (2): 228-247 or Therasse et al. (2000) J. Nat'l. Cancer Inst. 92(3): 205-216). In some embodiments, the amount a composition comprising the anti-WTMC construct (or construct combination) administered to an individual in need thereof (for example when administered as a single agent) is sufficient to produce an overall response rate of more than about any one of 20%, 25%, 30%, 35%, 40%, 45%, 50%, 55%, 60%, 64%, 65%, 70%, 75%, 80%, 85%, or 90% among a population of individuals treated with a pharmaceutical composition comprising an anti-WTMC construct (or construct combination) described herein. Responses of an individual to the treatment of the methods described herein can be determined, for example, based on RECIST levels.

In some embodiments, the amount of the pharmaceutical composition administered to an individual in need thereof is sufficient to prolong progression-free survival of the individual. In some embodiments, the amount of the composition administered to an individual in need thereof is sufficient to prolong overall survival of the individual. In some embodiments, the amount of the composition administered to an individual in need thereof is sufficient to produce clinical benefit rate of more than about any of 50%, 60%, 70%, or 77% among a population of individuals treated with the anti-WTMC construct composition (e.g., full-length anti-WTMC antibody, multispecific anti-WTMC construct, an anti-WTMC chimeric antigen receptor construct (CAR), an anti-WTMC chimeric antibody-T cell receptor construct (caTCR), an anti-WTMC chimeric signaling receptor construct (CSR), an anti-WTMC immunoconjugate, or any other anti-WTMC construct or construct combination described in further detail elsewhere herein).

In some embodiments, the amount of the composition administered to an individual in need thereof (e.g., as a single agent or in combination with a second, third, and/or fourth agent), is sufficient to decrease the size of a tumor, decrease the number of cancer cells, or decrease the growth rate of a tumor by at least about any of 10%, 20%, 30%, 40%, 50%, 60%, 70%, 80%, 90%, 95% or 100% (including any range in between these values), as compared to the corresponding tumor size, number of cancer cells, or tumor growth rate in the same subject prior to treatment, or as compared to the corresponding activity in other subjects not receiving the treatment. Standard methods can be used to measure the magnitude of this effect, such as in vitro assays with purified enzyme, cell-based assays, animal models, or human testing.

In some embodiments, the amount of the anti-WTMC construct (e.g., full-length anti-WTMC antibody, multispecific anti-WTMC construct, an anti-WTMC chimeric antigen receptor construct (CAR), an anti-WTMC chimeric antibody-T cell receptor construct (caTCR), an anti-WTMC chimeric signaling receptor construct (CSR), an anti-WTMC immunoconjugate, or any other anti-WTMC construct or construct combination described in further detail elsewhere herein) in the pharmaceutical composition is below the level that induces a toxicological effect. In some embodiments, the amount of the anti-WTMC construct (e.g., full-length anti-WTMC antibody, multispecific anti-WTMC construct, an anti-WTMC chimeric antigen receptor construct (CAR), an anti-WTMC chimeric antibody-T cell receptor construct (caTCR), an anti-WTMC chimeric signaling receptor construct (CSR), an anti-WTMC immunoconjugate, or any other anti-WTMC construct or construct combination described in further detail elsewhere herein) in the pharmaceutical composition is at a level where a potential side effect can be controlled or tolerated when the composition is administered to the individual.

In some embodiments, the amount of the pharmaceutical composition administered to an individual in need thereof is close to a maximum tolerated dose (MTD) of the composition following the same dosing regimen. In some embodiments, the amount of the composition is more than about any of 80%, 90%, 95%, or 98% of the MTD.

In some embodiments, the amount of an anti-WTMC construct (e.g., full-length anti-WTMC antibody, multispecific anti-WTMC construct, an anti-WTMC chimeric antigen receptor construct (CAR), an anti-WTMC chimeric antibody-T cell receptor construct (caTCR), an anti-WTMC chimeric signaling receptor construct (CSR), an anti-WTMC immunoconjugate, or any other anti-WTMC construct or construct combination described in further detail elsewhere herein) in the pharmaceutical composition is included in a range of about 0.001 μg to about 1000 μg.

In some embodiments, the effective amount of an anti-WTMC construct (e.g., full-length anti-WTMC antibody, multispecific anti-WTMC construct, an anti-WTMC immunoconjugate, or any other anti-WTMC construct or construct combination described in further detail elsewhere herein) in the composition is in the range of about 0.1 g/kg to about 100 mg/kg of total body weight.

A pharmaceutical composition comprising an anti-WTMC construct or construct combination described herein may be administered to an individual (such as human) via any known available route, including, for example, intravenous, intraportal, intra-arterial, intraperitoneal, intrahepatic, hepatic arterial infusion, intrapulmonary, oral, inhalation, intravesicular, intramuscular, intra-tracheal, subcutaneous, intraocular, intrathecal, transmucosal, and transdermal. In some embodiments, a sustained continuous release formulation of a pharmaceutical composition comprising an anti-WTMC construct described herein can be used.

### Anti-WTMC Effector Cell Therapy

In some embodiments, a method of treating a disease or disorder associated with expression, aberrant expression and/or aberrant activity of WT1 (e.g., WT1-RMF) comprises using an anti-WTMC effector cell (e.g., an anti-WTMC CAR effector cell, an anti-WTMC CAR plus tandem di-scFv effector cell, an anti-WTMC CAR plus CSR effector cell, an anti-WTMC caTCR effector cell, an anti-WTMC caTCR plus tandem di-scFv effector cell, and/or an anti-WTMC caTCR plus CSR effector cell) to redirect the specificity of an effector cell (such as a primary T cell) to a complex comprising WT1 peptide (e.g., WT1-RMF) and a MHC class I protein. In some embodiments, the WT1 peptide (e.g., WT1-RMF) comprises SEQ ID NO: 113. In some embodiments, the anti-WTMC effector cell specifically recognizes amino acid residues FPNA (SEQ ID NO: 349) of SEQ ID NO: 113. In some embodiments, the MHC class I protein is HLA-A*02:01. Thus, provided herein is a method of stimulating an effector cell-mediated response (such as a T cell-mediated immune response) to a target cell population and/or tissue (e.g., a target cell population and/or tissue comprising WTMC-expressing cells) in an individual, which method comprises the step of administering an anti-WTMC effector cell (such as a T cell) described herein to the individual.

Anti-WTMC effector cells (such as T cells), such as those described in further detail elsewhere herein, can be infused to an individual in need thereof (e.g., an individual who has or is suspected of having a disease or disorder associated with expression, aberrant expression, and/or aberrant activity of WT1 (e.g., WT1-RMF), such as cancer). The infused anti-WTMC effector cell is able to kill the target-expressing cells in the individual. Unlike therapeutic antibodies, anti-WTMC effector cells (such as T cells) are able to replicate in vivo, resulting in long-term persistence that can lead to sustained tumor control. In some embodiments, anti-WTMC effector cells (such as T cells) develop into specific memory T cells that can be reactivated to inhibit any additional tumor formation or growth.

The anti-WTMC effector cells (such as T cells) described herein may also serve as a type of vaccine for ex vivo immunization and/or in vivo therapy in an individual. In some embodiments, the individual is a mammal. In some embodiments, the mammal is a human or a non-human primate (such as a rhesus monkey or a cynomolgous monkey).

With respect to ex vivo immunization, of least one of the following occurs in vitro prior to administering the cell into the individual: i) expansion of the cells, ii) introducing a nucleic acid encoding an anti-WTMC CAR or an anti-WTMC caTCR to the cells, and/or iii) cryopreservation of the cells.

Ex vivo procedures are well known in the art and are discussed more fully below. Briefly, cells (e.g., T cells, such as αβ T cells, a γδ T cells, cytotoxic T cells, helper T cells, or natural killer T cells) are isolated from an individual (e.g., a mammal, preferably a human) and genetically modified (i.e., transduced or transfected, such as in vitro) with a nucleic acid, a set of nucleic acids, a vector, or a set of vectors encoding an anti-WTMC CAR, an anti-WTMC caTCR, anti-WTMC tandem multispecific scFv (such as an anti-WTMC tandem di-scFv), and/or anti-WTMC CSR disclosed herein.

In some embodiments, the cell (e.g., a T cell, such as an αβ T cell, a γδ T cell, a cytotoxic T cell, a helper T cell, or a natural killer T cell) has been genetically modified (i.e., transduced or transfected, such as in vitro) with the nucleic acid, the set of nucleic acids, the vector, or the set of vectors encoding anti-WTMC CAR, and is further genetically modified (i.e., transduced or transfected, such as in vitro) with a nucleic acid, a set of nucleic acids, a vector, or a set of vectors encoding a multispecific scFv (such as a tandem di-scFv) or a CSR. In some embodiments, the cell (e.g., a T cell, such as an αβ T cell, a T6 T cell, a cytotoxic T cell, a helper T cell, or a natural killer T cell) been genetically modified (i.e., transduced or transfected, such as in vitro) with a nucleic acid, a set of nucleic acids, a vector, or a set of vectors that encode an anti-WTMC CAR and a multispecific scFv (such as a tandem di-scFv). In some embodiments, the cell (e.g., a T cell, such as an αβ T cell, a γδ T cell, a cytotoxic T cell, a helper T cell, or a natural killer T cell) been genetically modified (i.e., transduced or transfected, such as in vitro) with a nucleic acid, a set of nucleic acids, a vector, or a set of vectors that encode an anti-WTMC CAR and a CSR. In some embodiments, the multispecific scFv (e.g., a tandem di-scFv) targets a WTMC complex. In some embodiments, the multispecific scFv (e.g., a tandem di-scFv) targets a different antigen (e.g., an antigen other than WT1 (e.g., WT1-RMF)). In some embodiments, the multispecific scFv (e.g., a tandem di-scFv) binds to a target ligand other than a WTMC complex. In some embodiments, the CSR targets a WTMC complex. In some embodiments, the CSR binds to a target ligand other than a WTMC complex.

In some embodiments, the cell (e.g., a T cell, such as an αβ T cell, a γδ T cell, a cytotoxic T cell, a helper T cell, or a natural killer T cell) has been genetically modified (i.e., transduced or transfected, such as in vitro) with the nucleic acid, the set of nucleic acids, the vector, or the set of vectors encoding anti-WTMC caTCR, and is further genetically modified (i.e., transduced or transfected, such as in vitro) with a nucleic acid, a set of nucleic acids, a vector, or a set of vectors encoding a multispecific scFv (such as a tandem di-scFv) or a CSR. In some embodiments, the cell (e.g., a T cell, such as an αβ T cell, a γδ T cell, a cytotoxic T cell, a helper T cell, or a natural killer T cell) been genetically modified (i.e., transduced or transfected, such as in vitro) with a nucleic acid, a set of nucleic acids, a vector, or a set of vectors that encode an anti-WTMC caTCR and a multispecific scFv (such as a tandem di-scFv). In some embodiments, the cell (e.g., a T cell, such as an αβ T cell, a γδ T cell, a cytotoxic T cell, a helper T cell, or a natural killer T cell) been genetically modified (i.e., transduced or transfected, such as in vitro) with a nucleic acid, a set of nucleic acids, a vector, or a set of vectors that encode an anti-WTMC caTCR and a CSR. In some embodiments, the multispecific scFv (e.g., a tandem di-scFv) targets a WTMC complex. In some embodiments, the multispecific scFv (e.g., a tandem di-scFv) targets a different antigen (e.g., an antigen other than WT1 (e.g., WT1-RMF)). In some embodiments, the multispecific scFv (e.g., a tandem di-scFv) binds to a target ligand other than a WTMC complex. In some embodiments, the CSR targets a WTMC complex. In some embodiments, the CSR binds to a target ligand other than a WTMC complex. In some embodiments, the cell (e.g., a T cell, such as an αβ T cell, a γδ T cell, a cytotoxic T cell, a helper T cell, or a natural killer T cell) has been genetically modified (i.e., transduced or transfected, such as in vitro) with the nucleic acid, the set of nucleic acids, the vector, or the set of vectors encoding the anti-WTMC tandem multispecific scFv (such as a tandem di-scFv) or the anti-WTMC CSR, and is further genetically modified (i.e., transduced or transfected, such as in vitro) with a nucleic acid, a set of nucleic acids, a vector, or a set of vectors encoding a CAR. In some embodiments, the cell (e.g., a T cell, such as an αβ T cell, a γδ T cell, a cytotoxic T cell, a helper T cell, or a natural killer T cell) been genetically modified (i.e., transduced or transfected, such as in vitro) with a nucleic acid, a set of nucleic acids, a vector, or a set of vectors that encode an anti-WTMC tandem multispecific scFv (such as a tandem di-scFv). In some embodiments, the cell (e.g., a T cell, such as an αβ T cell, a γδ T cell, a cytotoxic T cell, a helper T cell, or a natural killer T cell) been genetically modified (i.e., transduced or transfected, such as in vitro) with a nucleic acid, a set of nucleic acids, a vector, or a set of vectors that encode an anti-WTMC tandem multispecific scFv (such as a tandem di-scFv) or anti-WTMC CSR and a CAR. In some embodiments, the CAR targets a WTMC complex. In some embodiments, the CAR binds to a target ligand other than a WTMC complex.

In some embodiments, the cell (e.g., a T cell, such as an αβ T cell, a γδ T cell, a cytotoxic T cell, a helper T cell, or a natural killer T cell) has been genetically modified (i.e., transduced or transfected, such as in vitro) with the nucleic acid, the set of nucleic acids, the vector, or the set of vectors encoding the anti-WTMC tandem multispecific scFv (such as a tandem di-scFv) or the anti-WTMC CSR, and is further genetically modified (i.e., transduced or transfected, such as in vitro) with a nucleic acid, a set of nucleic acids, a vector, or a set of vectors encoding a caTCR. In some embodiments, the cell (e.g., a T cell, such as an αβ T cell, a γδ T cell, a cytotoxic T cell, a helper T cell, or a natural killer T cell) been genetically modified (i.e., transduced or transfected, such as in vitro) with a nucleic acid, a set of nucleic acids, a vector, or a set of vectors that encode an anti-WTMC tandem multispecific scFv (such as a tandem di-scFv). In some embodiments, the cell (e.g., a T cell, such as an αβ T cell, a γδ T cell, a cytotoxic T cell, a helper T cell, or a natural killer T cell) been genetically modified (i.e., transduced or transfected, such as in vitro) with a nucleic acid, a set of nucleic acids, a vector, or a set of vectors that encode an anti-WTMC tandem multispecific scFv (such as a tandem di-scFv) or anti-WTMC CSR and a caTCR. In some embodiments, the caTCR targets a WTMC complex. In some embodiments, the caTCR binds to a target ligand other than a WTMC complex. In some embodiments, the cell is an αβ T cell that has been genetically modified with a nucleic acid, a set of nucleic acids, a vector, or a set of vectors that encode an anti-WTMC caTCR comprising γδ TCR transmembrane sequences. In some embodiments, the cell is an αβ T cell comprising and/or expressing an anti-WTMC caTCR comprising γδ TCR transmembrane sequences.

In some embodiments, the cells (e.g., T cells, such as αβ T cells, γδ T cells, cytotoxic T cells, helper T cells, or natural killer T cells) that have been genetically modified (i.e., transduced or transfected, such as in vitro) as described above are administered to a recipient. In some embodiments, the recipient is a mammal, such as a human, e.g., a human who has or is suspected of having the WT1-associated disease. In some embodiments, the recipient to whom the genetically modified cells are administered is the individual from whom the cells were obtained. Such a genetically modified immune cell is referred to as an “autologous anti-WTMC effector cell.” In some embodiments, the recipient to whom the genetically modified immune cells are administered is not the individual from whom the cells were obtained. Such a genetically modified immune cell is referred to as a “heterologous anti-WTMC effector cell.” In some embodiments, the heterologous anti-WTMC effector cell is allogeneic, syngeneic, or xenogeneic with respect to the recipient.

The procedure for ex vivo expansion of hematopoietic stem and progenitor cells is described in U.S. Pat. No. 5,199,942, incorporated herein by reference in its entirety. Other suitable methods are also known in the art, and the present disclosure is not limited to any particular method of ex vivo expansion of the cells. Briefly, ex vivo culture and expansion of T cells comprises: (1) collecting CD34⁺ hematopoietic stem and progenitor cells from an individual (e.g., a mammal such as a human) from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present disclosure also provides compositions and methods for in vivo immunization to elicit an immune response directed against an antigen in an individual in need thereof (e.g., an individual who has or is suspected of having a WT1-associated disease, such as cancer).

The anti-WTMC effector cells (such as T cells) of the present disclosure may be administered either alone, or as a pharmaceutical composition in combination with diluents and/or with other components such as IL-2 or other cytokines or cell populations. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, effector cell (such as T cell) compositions are formulated for intravenous administration.

The precise amount of the anti-WTMC effector cell (such as T cell) of the present disclosure to be administered to an individual in need thereof can be determined by a physician with consideration of the individual's age, weight, tumor size, stage and/or severity of the disease, presence or absence of metastasis, condition of the individual, and other factors. In some embodiments, a pharmaceutical composition comprising anti-WTMC effector cells (such as T cells) of the present disclosure is administered at a dosage of about 10⁴ to about 10⁹ cells/kg body weight, such any of about 10⁴ to about 10⁵, about 10⁵ to about 10⁶, about 10⁶ to about 10⁷, about 10⁷ to about 10⁸, or about 10⁸ to about 10⁹ cells/kg body weight, including all integer values within those ranges. Anti-WTMC effector cell (such as T cell) compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regimen for a particular patient can readily be determined by one skilled in the art of medicine by monitoring the patient for signs of disease and adjusting the treatment accordingly.

In some embodiments, it may be desirable to administer activated anti-WTMC effector cells (such as T cells) described herein to an individual, subsequently redraw blood (or have an apheresis performed), activate the anti-WTMC effector cells (e.g., anti-WTMC T cells) described herein obtained from the redrawn blood, and reinfuse the individual with the activated and expanded anti-WTMC effector cells (e.g., anti-WTMC T cells). In some embodiments, this process is carried out multiple times every few weeks. In some embodiments, the anti-WTMC effector cells (e.g., anti-WTMC T cells) are activated from blood draws of from 10 cc to 400 cc. In some embodiments, the anti-WTMC effector cells (e.g., anti-WTMC T cells) are activated from blood draws of 20 cc, 30 cc, 40 cc, 50 cc, 60 cc, 70 cc, 80 cc, 90 cc, or 100 cc.

The administration anti-WTMC effector cells (e.g., anti-WTMC T cells) described herein may be carried out in any convenient manner, including by aerosol inhalation, injection, ingestion, transfusion, implantation or transplantation. Compositions comprising anti-WTMC effector cells (e.g., anti-WTMC T cells) described herein may be administered to a patient subcutaneously, intradermally, subcutaneously, intratumorally, intranodally, intramedullary, intramuscularly, by intravenous (i.v.) injection, or intraperitoneally. In some embodiments, compositions comprising anti-WTMC effector cells (e.g., anti-WTMC T cells) of the present disclosure are administered by i.v. injection directly into a tumor, lymph node, or site of disease.

Provided are methods of treating a WT1-associated disease in an individual that comprise administering to the individual an effective amount of a composition comprising an anti-WTMC effector cell (e.g., anti-WTMC T cell) of the present disclosure. In some embodiments, the WT1-associated disease is cancer. In some embodiments, the cancer is, for example, prostate cancer (such as hormone-refractory or metastatic prostate cancer), renal cell cancer cell (such as clear cell renal cell cancer), uterine cancer, or liver cancer. In some embodiments, the individual is human. In some embodiments, the individual to whom the composition comprising an anti-WTMC effector cell (e.g., anti-WTMC T cell) is administered is an individual who has (e.g., has been diagnosed with) or is suspected of having a WT1-associated disease. In some embodiments, the disease or disorder associated with expression, aberrant expression and/or aberrant activity of WT1 (e.g., WT1-RMF) is refractory to at least one conventional treatment. In some embodiments, the individual to whom the composition comprising an anti-WTMC effector cell (e.g., anti-WTMC T cell) is administered is an individual who has (e.g., has been diagnosed with) a disease or disorder associated with expression, aberrant expression and/or aberrant activity of WT1 (e.g., WT1-RMF) and has relapsed following at least one conventional treatment for the diseases or disorders associated with expression, aberrant expression and/or aberrant activity of WT1 (e.g., WT1-RMF).

Thus, for example, in some embodiments, there is provided a method of treating a disease or disorder associated with expression, aberrant expression and/or aberrant activity of WT1 (e.g., WT1-RMF) in an individual comprising administering to the individual an effective amount of a composition comprising an effector cell (such as a T cell) expressing an anti-WTMC CAR, an anti-WTMC caTCR, an anti-WTMC caTCR plus tandem di-scFv, and/or an anti-WTMC caTCR plus CSR comprising an extracellular domain comprising an anti-WTMC antibody moiety of the present disclosure. In some embodiments, the WT1 peptide (e.g., WT1-RMF) comprises SEQ ID NO: 113. In some embodiments, the antibody moiety specifically recognizes amino acid residues FPNA (SEQ ID NO: 349) of SEQ ID NO: 113. In some embodiments, the disease or disorder associated with expression, aberrant expression and/or aberrant activity of WT1 (e.g., WT1-RMF) is cancer. In some embodiments, the cancer is a WT1-positive cancer. In some embodiments, the cancer is chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma. In some embodiments, the MHC class I protein is HLA-A02. In some embodiments, the MHC class I protein is HLA-A*02:01. In some embodiments, the individual is human.

In some embodiments, there is provided a method of treating a disease or disorder associated with expression, aberrant expression and/or aberrant activity of WT1 (e.g., WT1-RMF) in an individual comprising administering to the individual an effective amount of a composition comprising an effector cell (such as a T cell) expressing a CSR and an anti-WTMC CAR specifically binding to a complex comprising a WT1 peptide (e.g., WT1-RMF) and an MHC class I protein. In some embodiments, the WT1 peptide (e.g., WT1-RMF) comprises SEQ ID NO: 113. In some embodiments, the anti-WTMC CAR specifically recognizes amino acid residues FPNA (SEQ ID NO: 349) of SEQ ID NO: 113. In some embodiments, the MHC class I protein is HLA-A*02:01. In some embodiments, the CSR is an anti-CD33 CSR that specifically binds to CD33. In some embodiments, the CSR is an anti-CD371, anti-CD123, or anti-CD15 CSR that specifically binds to CD371, CD123, or CD15. In some embodiments, the CSR is a bispecific CSR that specifically binds to both CD33 and any one of CD371, CD123, or CD15. In some embodiments, the effector cell expressing the anti-WTMC CAR and the anti-CD33 CSR, anti-CD371 CSR, anti-CD123 CSR, anti-CD15 CSR, or the anti-CD33×anti-CD371/anti-CD123/or anti-CD15 bispecific CSR is used to treat a WT1-positive cancer (such as chronic myelocytic leukemia, multiple myeloma (MM), acute lymphoblastic leukemia (ALL), acute myeloid/myelogenous leukemia (AML), myelodysplastic syndrome (MD), mesothelioma, ovarian cancer, gastrointestinal cancers, breast cancer, prostate cancer or glioblastoma). In some embodiments, the effector cell expressing the anti-WTMC caTCR and the anti-CD33 CSR, anti-CD371 CSR, anti-CD123 CSR, anti-CD15 CSR, or the anti-CD33×anti-CD371/anti-CD123/or anti-CD15 bispecific CSR is used to treat a WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma).

In some embodiments, the CSR is an anti-MUC16 CSR that specifically binds to a MUC16 target ligand. In some embodiments, the effector cell expressing the anti-WTMC CAR and the anti-MUC16 CSR is used to treat cancer type. In some embodiments, the CSR is an anti-HER2 CSR that specifically binds to a HER2 target ligand. In some embodiments, the effector cell expressing the anti-WTMC CAR and the anti-HER2 CSR is used to treat cancer type.

In some embodiments, is provided a method of treating a disease or disorder associated with expression, aberrant expression and/or aberrant activity of WT1 (e.g., WT1-RMF) in an individual comprising administering to the individual an effective amount of a composition comprising an effector cell (such as a T cell) expressing an anti-WTMC caTCR and a CSR. In some embodiments, the anti-WTMC caTCR specifically binds to a complex comprising a WT1 peptide (e.g., WT1-RMF) and an MHC class I protein. In some embodiments, the WT1 peptide (e.g., WT1-RMF) comprises SEQ ID NO: 113. In some embodiments, the anti-WTMC caTCR specifically recognizes amino acid residues FPNA (SEQ ID NO: 349) of SEQ ID NO: 113. In some embodiments, the MHC class I protein is HLA-A*02:01. In some embodiments, the CSR is an anti-CD33 CSR that specifically binds to a CD33 target ligand. In some embodiments, the CSR is an anti-CD371, anti-CD123, or anti-CD15 CSR that specifically binds to a CD371, CD123, or CD15 target ligand.

In some embodiments, the CSR is a bispecific CSR that specifically binds to both CD33 and any one of CD371, CD123, or CD15. In some embodiments, the anti-WTMC caTCR and the anti-CD33 CSR, anti-CD371 CSR, anti-CD123 CSR, anti-CD15 CSR, or the anti-CD33×anti-CD371/anti-CD123/or anti-CD15 bispecific CSR is used to treat a WT1-positive cancer (such as chronic myelocytic leukemia, multiple myeloma (MM), acute lymphoblastic leukemia (ALL), acute myeloid/myelogenous leukemia (AML), myelodysplastic syndrome (MDS)). In some embodiments, the effector cell expressing the anti-WTMC caTCR and the anti-CD33 CSR, anti-CD371 CSR, anti-CD123 CSR, anti-CD15 CSR, or the anti-CD33×anti-CD371/anti-CD123/or anti-CD15 bispecific CSR is used to treat a WT1-positive cancer (such as chronic myelocytic leukemia, multiple myeloma (MM), acute lymphoblastic leukemia (ALL), acute myeloid/myelogenous leukemia (AML), myelodysplastic syndrome (MD)).

In some embodiments, the CSR is an anti-MUC16 CSR that specifically binds to a MUC16 target ligand. In some embodiments, the effector cell expressing the anti-WTMC caTCR and the anti-MUC16 CSR is used to treat cancer type. In some embodiments, the CSR is an anti-HER2 CSR that specifically binds to a HER2 target ligand. In some embodiments, the effector cell expressing the anti-WTMC caTCR and the anti-HER2 CSR is used to treat cancer type.

In some embodiments, is provided a method of treating a disease or disorder associated with expression, aberrant expression and/or aberrant activity of WT1 (e.g., WT1-RMF) in an individual comprising administering to the individual an effective amount of a composition comprising an effector cell (such as a T cell) expressing an anti-WTMC CSR and a CAR. In some embodiments, the anti-WTMC CSR specifically binds to a complex comprising a WT1 peptide (e.g., WT1-RMF) and an MHC class I protein. In some embodiments, the WT1 peptide (e.g., WT1-RMF) comprises SEQ ID NO: 113. In some embodiments, the anti-WTMC CSR specifically recognizes amino acid residues FPNA (SEQ ID NO: 349) of SEQ ID NO: 113. In some embodiments, the MHC class I protein is HLA-A*02:01. In some embodiments, the CAR is an anti-CD33 CAR that specifically binds to a CD33 target ligand. In some embodiments, the CAR is an anti-CD371 CAR, anti-CD123 CAR, or anti-CD15 CAR that specifically binds to a CD371, CD123, or CD15 target ligand. In some embodiments, the CAR is bispecific CAR that specifically binds to both CD33 and any one of CD371, CD123, or CD15 target ligand.

In some embodiments, the effector cell expressing the anti-WTMC CSR and the anti-CD33 CAR, anti-CD371 CAR, anti-CD123 CAR, anti-CD15 CAR, or the anti-CD33×anti-CD371/anti-CD123/or anti-CD15 bispecific CAR is used to treat a WT1-positive cancer (such as chronic myelocytic leukemia, multiple myeloma (MM), acute lymphoblastic leukemia (ALL), acute myeloid/myelogenous leukemia (AML), myelodysplastic syndrome (MDS)). In some embodiments, the anti-WTMC CSR and the anti-CD33 CAR, anti-CD371 CAR, anti-CD123 CAR, anti-CD15 CAR, or the anti-CD33×anti-CD371/anti-CD123/or anti-CD15 bispecific CAR is used to treat a WT1-positive cancer (such as chronic myelocytic leukemia, multiple myeloma (MM), acute lymphoblastic leukemia (ALL), acute myeloid/myelogenous leukemia (AML), myelodysplastic syndrome (MD)).

In some embodiments, the CAR is an anti-MUC16 CAR that specifically binds to a MUC16 target ligand. In some embodiments, the effector cell expressing the anti-WTMC CSR and the anti-MUC16 CAR is used to treat cancer type. In some embodiments, the CAR is an anti-HER2 CAR that specifically binds to a HER2 target ligand. In some embodiments, the effector cell expressing the anti-WTMC CSR and the anti-HER2 CAR is used to treat cancer type. In some embodiments, the CAR is an anti-WT1/MHC CAR that specifically binds to a complex comprising a WT1 peptide (e.g., WT1-RMF) and an MHC class I protein. In some embodiments, the effector cell expressing the anti-WTMC CSR and the anti-WT1/MHC CAR is used to treat a WT1-positive cancer (such as chronic myelocytic leukemia, multiple myeloma (MM), acute lymphoblastic leukemia (ALL), acute myeloid/myelogenous leukemia (AML), myelodysplastic syndrome (MD), mesothelioma, ovarian cancer, gastrointestinal cancers, breast cancer, prostate cancer or glioblastoma). In some embodiments, the effector cell expressing the anti-WTMC CSR and the anti-WT1/MHC CAR is used to treat a WT1-positive cancer (such as chronic myelocytic leukemia, multiple myeloma (MM), acute lymphoblastic leukemia (ALL), acute myeloid/myelogenous leukemia (AML), myelodysplastic syndrome (MD), mesothelioma, ovarian cancer, gastrointestinal cancers, breast cancer, prostate cancer or glioblastoma).

In some embodiments, is provided a method of treating a disease or disorder associated with expression, aberrant expression and/or aberrant activity of WT1 (e.g., WT1-RMF) in an individual comprising administering to the individual an effective amount of a composition comprising an effector cell (such as a T cell) expressing an anti-WTMC CSR and a caTCR. In some embodiments, the anti-WTMC CSR specifically binds to a complex comprising a WT1 peptide (e.g., WT1-RMF) and an MHC class I protein. In some embodiments, the WT1 peptide (e.g., WT1-RMF) comprises SEQ ID NO: 113. In some embodiments, the anti-WTMC CSR specifically recognizes amino acid residues FPNA (SEQ ID NO: 349) of SEQ ID NO: 113. In some embodiments, the MHC class I protein is HLA-A*02:01. In some embodiments, the caTCR is an anti-CD33 caTCR that specifically binds to a CD33 target ligand. In some embodiments, the caTCR is an anti-CD371 caTCR, anti-CD123 caTCR, anti-CD15 caTCR that specifically binds to a CD371, CD123 or CD15 target ligand. In some embodiments, the effector cell expressing the anti-WTMC CSR and the anti-CD33 caTCR, anti-CD371 caTCR, anti-CD123 caTCR, anti-CD15 caTCR, or the anti-CD33×anti-CD371/anti-CD123/or anti-CD15 bispecific caTCR is used to treat a WT1-positive cancer (such as chronic myelocytic leukemia, multiple myeloma (MM), acute lymphoblastic leukemia (ALL), acute myeloid/myelogenous leukemia (AML), myelodysplastic syndrome (MD)). In some embodiments, the anti-WTMC CSR and the anti-CD33 caTCR, anti-CD371 caTCR, anti-CD123 caTCR, anti-CD15 caTCR, or the anti-CD33×anti-CD371/anti-CD123/or anti-CD15 bispecific caTCR is used to treat a WT1-positive cancer (such as chronic myelocytic leukemia, multiple myeloma (MM), acute lymphoblastic leukemia (ALL), acute myeloid/myelogenous leukemia (AML), myelodysplastic syndrome (MDS)).

In some embodiments, the caTCR is an anti-MUC16 caTCR that specifically binds to a MUC16 target ligand. In some embodiments, the effector cell expressing the anti-WTMC CSR and the anti-MUC16 caTCR is used to treat cancer type. In some embodiments, the caTCR is an anti-HER2 caTCR that specifically binds to a HER2 target ligand. In some embodiments, the effector cell expressing the anti-WTMC CSR and the anti-HER2 caTCR is used to treat cancer type. In some embodiments, the caTCR is an anti-WT1/MHC caTCR that specifically binds to a complex comprising a WT1 peptide (e.g., WT1-RMF) and an MHC class I protein. In some embodiments, the effector cell expressing the anti-WTMC CSR and the anti-WT1/MHC caTCR is used to treat a WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma). In some embodiments, the effector cell expressing the anti-WTMC CSR and the anti-WT1/MHC caTCR is used to treat a WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma).

In some embodiments, is provided a method of treating a disease or disorder associated with expression, aberrant expression and/or aberrant activity of WT1 (e.g., WT1-RMF) in an individual comprising administering to the individual an effective amount of a composition comprising an effector cell (such as a T cell) expressing an anti-WTMC CSR and a TCR. In some embodiments, the anti-WTMC CSR specifically binds to a complex comprising a WT1 peptide (e.g., WT1-RMF) and an MHC class I protein. In some embodiments, the WT1 peptide (e.g., WT1-RMF) comprises SEQ ID NO: 113. In some embodiments, the anti-WTMC CSR specifically recognizes amino acid residues FPNA (SEQ ID NO: 349) of SEQ ID NO: 113. In some embodiments, the MHC class I protein is HLA-A*02:01. In some embodiments, the TCR is an anti-WT1/MHC TCR that specifically binds to a complex comprising a WT1 peptide (e.g., WT1-RMF) and an MHC class I protein. In some embodiments, the effector cell expressing the anti-WTMC CSR and the anti-WT1/MHC TCR is used to treat a WT1-positive cancer (such as chronic myelocytic leukemia, multiple myeloma (MM), acute lymphoblastic leukemia (ALL), acute myeloid/myelogenous leukemia (AML), myelodysplastic syndrome (MD), mesothelioma, ovarian cancer, gastrointestinal cancers, breast cancer, prostate cancer or glioblastoma). In some embodiments, the effector cell expressing the anti-WTMC CSR and the anti-WT1/MHC TCR is used to treat a WT1-positive cancer (such as chronic myelocytic leukemia, multiple myeloma (MM), acute lymphoblastic leukemia (ALL), acute myeloid/myelogenous leukemia (AML), myelodysplastic syndrome (MD), mesothelioma, ovarian cancer, gastrointestinal cancers, breast cancer, prostate cancer or glioblastoma).

### Cancers

The anti-WTMC constructs and anti-WTMC chimeric receptor effector cells in some embodiments can be useful for treating WT1-related cancer. Cancers that may be treated using any of the methods described herein include tumors that are not vascularized, or not yet substantially vascularized, as well as vascularized tumors. The cancers may comprise non-solid tumors (such as hematological tumors, for example, chronic myelocytic leukemia, multiple myeloma (MM), acute lymphoblastic leukemia (ALL), acute myeloid/myelogenous leukemia (AML), myelodysplastic syndrome (MDS), and lymphomas) or may comprise solid tumors.

Types of cancers to be treated with the anti-WTMC constructs and anti-WTMC chimeric receptor effector cells of the present application include, but are not limited to, chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma. The cancers to be treated include adult tumors/cancers and pediatric tumors/cancers.

Solid tumors contemplated for treatment by any of the methods described herein include mesothelioma, ovarian cancer, gastrointestinal cancers, breast cancer, prostate cancer or glioblastoma.

In some embodiments, the WT1-related cancer is chronic myelocytic leukemia, multiple myeloma (MM), acute lymphoblastic leukemia (ALL), acute myeloid/myelogenous leukemia (AML), myelodysplastic syndrome (MD), mesothelioma, ovarian cancer, gastrointestinal cancers, breast cancer, prostate cancer or glioblastoma.

Cancer treatments can be evaluated, for example, by tumor regression, tumor weight or size shrinkage, time to progression, duration of survival, progression free survival, overall response rate, duration of response, quality of life, protein expression and/or activity. Approaches to determining efficacy of the therapy can be employed, including for example, measurement of response through radiological imaging.

### Methods for Diagnosis and Imaging Using Anti-WTMC Constructs

Labeled anti-WTMC constructs described herein (e.g., constructs that specifically bind to a complex comprising WT1 peptide (e.g., WT1-RMF) and a major histocompatibility (MHC) class I protein) may be used for diagnostic purposes to, e.g., detect, diagnose, monitor the progression of a WT1-associated disease or disorder, e.g., a disease or disorder associated with the expression, aberrant expression and/or activity of WT1 (e.g., WT1-RMF), and/or monitor a patients response to treatment for a WT1-associated disease. Exemplary WT1-associated diseases or disorders include any of the diseases and disorders described herein, such as cancer (e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)). For example, the anti-WTMC constructs described herein can be used in in situ, in vivo, ex vivo, and in vitro diagnostic assays or imaging assays.

In some embodiments, provided are methods of diagnosing a disease or disorder associated with expression or aberrant expression of WT1 (e.g., WT1-RMF) in an individual (e.g., a mammal, such as a human or a non-human primate, such as a rhesus monkey or a cynomolgus monkey). The methods comprise detecting cells that aberrantly express WT1 in the individual. In some embodiments, provided is a method of diagnosing a WT1-associated disease or disorder in an individual (e.g., a mammal, such as a human or a non-human primate, such as a rhesus monkey or a cynomolgus monkey) comprising (a) administering an effective amount of a labeled anti-WTMC construct described herein to the individual; and (b) determining the level of the label in the individual, such that a level of the label above a threshold level indicates that the individual has the disease or disorder. The threshold level can be determined by various methods, including, for example, by detecting the label according to the method of diagnosing described herein in a first set of individuals that have the disease or disorder and a second set of individuals that do not have the disease or disorder, and setting the threshold to a level that allows for discrimination between the first and second sets. In some embodiments, the threshold level is zero, and the method comprises determining the presence or absence of the label in the individual. In some embodiments, the method further comprises waiting for a time interval following the administering of step (a) to permit the labeled anti-WTMC construct to preferentially concentrate at sites in the individual where the WT1 is expressed (and for unbound labeled anti-WTMC construct to be cleared). In some embodiments, the method further comprises subtracting a background level of the label. Background level can be determined by various methods, including, for example, by detecting the label in the individual prior to administration of the labeled anti-WTMC construct, or by detecting the label according to the method of diagnosing described herein in an individual that does not have the disease or disorder. In some embodiments, the disease or disorder is cancer. In some embodiments, the cancer is for example, a WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma). In some embodiments, the individual is human. In some embodiments, the individual is suspected of having a disease or disorder associated with expression, aberrant expression and/or activity of WT1 (e.g., WT1-RMF).

In some embodiments, provided is a method of diagnosing a WT1-associated disease or disorder in an individual (e.g., a mammal, such as a human or a non-human primate, such as a rhesus monkey or a cynomolgus monkey), comprising (a) contacting a labeled anti-WTMC construct according to any of the embodiments described herein with a sample (such as homogenized tissue) obtained or derived from the individual; and (b) determining the number of cells bound with the labeled anti-WTMC construct in the sample, such that a value for the number of cells bound with the labeled anti-WTMC construct above a threshold level indicates that the individual has the disease or disorder. The threshold level can be determined by various methods, including, for example, by determining the number of cells bound with the labeled anti-WTMC construct according to the method of diagnosing described herein in a first set of individuals that have the disease or disorder and a second set of individuals that do not have the disease or disorder, and setting the threshold to a level that allows for discrimination between the first and second sets. In some embodiments, the threshold level is zero, and the method comprises determining the presence or absence of cells bound with the labeled anti-WTMC construct in the sample. In some embodiments, the method further comprises subtracting a background level of the number of cells bound with the labeled anti-WTMC construct. Background level can be determined by various methods, including, for example, by determining the number of cells bound with the labeled anti-WTMC construct in the individual prior to administration of the labeled anti-WTMC construct, or by determining the number of cells bound with the labeled anti-WTMC construct according to the method of diagnosing described herein in an individual that does not have the disease or disorder. In some embodiments, the disease or disorder is cancer. In some embodiments, the cancer is selected, for example, from the group consisting of chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma. In some embodiments, the cancer is metastatic. In some embodiments, the individual is human. In some embodiments, the individual is suspected of having a WT1-associated disease or disorder.

In some embodiments, there is provided a method of diagnosing a WT1-associated cancer in an individual (e.g., a mammal, such as a human or a non-human primate, such as a rhesus monkey or a cynomolgus monkey), comprising (a) contacting a labeled anti-WTMC construct according to any of the embodiments described herein with a tissue sample derived from the individual; and (b) determining the number of cells in the tissue sample bound with the labeled anti-WTMC construct, such that a value for the number of cells in the tissue sample bound with the labeled anti-WTMC construct above a threshold level indicates that the individual has a WT1-associated cancer. The threshold level can be determined by various methods, including, for example, by determining the number of cells bound with the labeled anti-WTMC construct according to the method of diagnosing described herein in tissue samples from a first set of individuals who have a WT1-associated and tissue samples from a second set of individuals who do not have a WT1-associated cancer, and setting the threshold to a level that allows for discrimination between the tissue samples from the first and second sets. In some embodiments, the threshold level is zero, and the method comprises determining the presence or absence of cells in the tissue sample bound with the labeled anti-WTMC antibody moiety. In some embodiments, the method further comprises subtracting a background level of the number of cells bound with the labeled anti-WTMC construct. Background level can be determined by various methods, including, for example, by determining the number of cells in the tissue sample bound with the labeled anti-WTMC construct in the individual prior to contacting with the labeled anti-WTMC construct, or by determining the number of cells in a tissue sample bound with the labeled anti-WTMC construct according to the method of diagnosing described herein, which tissue sample is obtained or derived from an individual that does not have a WT1-associated cancer. In some embodiments, the WT1-associated cancer is selected, for example, from the group consisting of chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma. In some embodiments, the individual is human. In some embodiments, the individual is suspected of having a WT1-associated cancer.

The anti-WTMC constructs provided herein may be used to assay levels of WT1 (e.g., WT1-RMF) in a biological sample using methods known to those of skill in the art. Suitable labels are known in the art and include enzyme labels, such as, glucose oxidase; radioisotopes, such as iodine (¹³¹I, ¹²⁵I, ¹²³I, ¹²¹I), carbon (¹⁴C), sulfur (³⁵S), tritium (³H), indium (^{115m}In, ^{113m}In, ¹¹²In ¹¹¹In), technetium (⁹⁹Tc, ^{99m}Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), samarium (¹⁵³Sm), lutetium (¹⁷⁷Lu), gadolinium (¹⁵⁹Gd), promethium (¹⁴⁹Pm), lanthanum (¹⁴⁰La), ytterbium (¹⁷⁵Yb), holmium (¹⁶⁶Ho), yttrium (⁹⁰Y), scandium (⁴⁷Sc), rhenium (¹⁸⁶Re, ¹⁸⁸Re), praseodymium (¹⁴²Pr), rhodium (¹⁰⁵Rh) and ruthenium (⁹⁷Ru); luminol; fluorescent labels, such as fluorescein and rhodamine; and biotin.

Techniques known in the art may be applied to labeled anti-WTMC constructs provided herein. Such techniques include, but are not limited to, the use of bifunctional conjugating agents (see e.g., U.S. Pat. Nos. 5,756,065; 5,714,631; 5,696,239; 5,652,361; 5,505,931; 5,489,425; 5,435,990; 5,428,139; 5,342,604; 5,274,119; 4,994,560; and 5,808,003). Aside from the above assays, various in vivo and ex vivo assays are available to the skilled practitioner. For example, one can expose cells within the body of the subject to an anti-WTMC construct which is optionally labeled with a detectable label, e.g., a radioactive isotope, and binding of the anti-WTMC antibody moiety to the cells can be evaluated, e.g., by external scanning for radioactivity or by analyzing a sample (e.g., a biopsy or other biological sample) derived from a subject previously exposed to the anti-WTMC construct.

### Articles of Manufacture and Kits

Provided herein are articles of manufacture that comprise materials useful for the diagnosis or treatment a disease or disorder associated with expression, aberrant expression and/or aberrant activity of Wilm's Tumor 1 (WT1), such as cancer (e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)), for delivering an anti-WTMC construct or construct combination to a cell expressing a complex comprising a WT1 peptide (e.g., WT1-RMF) and a MHC class I protein, or for isolation or detection of WTMC-expressing cells in an individual. The article of manufacture can comprise a container and a label or package insert on or associated with the container. Suitable containers include, for example, bottles, vials, syringes, etc. The containers may be formed from a variety of materials such as glass or plastic. Generally, the container holds a composition which is effective for diagnosing or treating a disease or disorder associated with expression, aberrant expression and/or aberrant activity of WT1 (e.g., WT1-RMF) described herein and may have a sterile access port (for example the container may be an intravenous solution bag or a vial having a stopper pierceable by a hypodermic injection needle). At least one active agent in the composition is an anti-WTMC construct or construct combination provided herein. The label or package insert indicates that the composition is used for treating the particular condition. The label or package insert will further comprise instructions for administering the anti-WTMC construct or construct combination (or, e.g., a composition comprising such construct or construct combination) to an individual in need thereof (e.g., an individual having or suspected of having a disease or disorder associated with expression, aberrant expression and/or aberrant activity of WT1 (e.g., WT1-RMF)). Articles of manufacture and kits comprising combinatorial therapies (e.g., one or more therapeutic agents in addition to an anti-WTMC construct or construct combination described herein) are also contemplated.

A package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating cancer associated with expression, aberrant expression and/or aberrant activity of WT1 (e.g., WT1-RMF) (e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)).

Additionally, the article of manufacture may further comprise a second container comprising a pharmaceutically acceptable buffer, such as bacteriostatic water for injection (BWFI), sterile water for injection (SWFI) phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a WT1-associated disease or disorder described herein, for delivering an anti-WTMC construct or construct combination to a cell comprising a complex comprising a WT1 peptide (e.g., WT1-RMF) and an MHC class I peptide, or for isolation or detection of WTMC-binding cells in an individual, optionally in combination with the articles of manufacture. Kits provided herein include one or more containers comprising a composition comprising an anti-WTMC construct or construct combination (or unit dosage form thereof and/or article of manufacture), and in some embodiments, further comprise another agent (such as the agents described herein) and/or instructions for use in accordance with any of the methods described herein. The kit may further comprise a description of the selection of individuals suitable for treatment. Instructions supplied in the kits herein are typically written instructions on a label or package insert (e.g., a paper sheet included in the kit), but machine-readable instructions (e.g., instructions carried on a magnetic or optical storage disk) are also acceptable.

For example, in some embodiments, the kit comprises a composition comprising an anti-WTMC construct (e.g., a full-length anti-WTMC antibody, a monospecific anti-WTMC construct, a multispecific anti-WTMC construct (such as a bispecific anti-WTMC antibody), or an anti-WTMC immunoconjugate) or an anti-WTMC construct combination (e.g., an anti-WTMC caTCR+anti-WTMC, an anti-WTMC caTCR+anti-WTMC CSR, an anti-WTMC CAR+CSR, an anti-WTMC CAR+anti-WTMC CSR, an anti-WTMC CSR+CAR, an anti-WTMC CSR+anti-WTMC caTCR). In some embodiments, the kit comprises a) a composition comprising an anti-WTMC construct or construct combination, and b) an effective amount of at least one other therapeutic agent. In some embodiments, the kit comprises a) a composition comprising an anti-WTMC construct or construct combination, and b) instructions for administering the composition to an individual for treatment of a WT1-associated disease, including for example chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma.

The anti-WTMC construct (or construct combination) and the other agent(s) can be present in separate containers or in a single container. For example, the kit may comprise one distinct composition or two or more compositions wherein one composition comprises an anti-WTMC construct or construct combination and another composition comprises another agent.

In some embodiments, the kit comprises a) a composition comprising an anti-WTMC construct (e.g., a full-length anti-WTMC antibody, a mono-specific anti-WTMC construct, a multispecific anti-WTMC construct (such as a bispecific anti-WTMC antibody), an anti-WTMC immunoconjugate, or other anti-WTMC construct described herein) or an anti-WTMC construct combination (e.g., an anti-WTMC caTCR+anti-WTMC, an anti-WTMC caTCR+anti-WTMC CSR, an anti-WTMC CAR+CSR, an anti-WTMC CAR+anti-WTMC CSR, an anti-WTMC CSR+CAR, an anti-WTMC CSR+anti-WTMC caTCR), and b) instructions for combining the anti-WTMC construct or construct combination with cells (such as cells, e.g., immune cells, derived from an individual) to form a composition comprising anti-WTMC construct-cell conjugates and administering the anti-WTMC construct-cell conjugate composition to the individual for treatment of a disease associated with expression, aberrant expression and/or aberrant activity of WT1 (e.g., WT1-RMF) (e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)). In some embodiments, the kit comprises a) a composition comprising an anti-WTMC construct or construct combination (such as described herein), and b) a cell (such as a cytotoxic cell). In some embodiments, the kit comprises a) a composition comprising an anti-WTMC construct or construct combination (such as described herein), b) a cell (such as a cytotoxic cell), and c) instructions for combining the anti-WTMC construct or construct combination with the cell to form a composition comprising anti-WTMC construct-cell conjugates and administering the anti-WTMC construct-cell conjugate composition to an individual for the treatment of a disease associated with expression, aberrant expression and/or aberrant activity of WT1 (e.g., WT1-RMF) (e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)). In some embodiments, the kit comprises a composition comprising an anti-WTMC construct or construct combination (such as described herein) in association with a cell (such as a cytotoxic cell). In some embodiments, the kit comprises a) a composition comprising an anti-WTMC construct or construct combination (such as described herein) in association with a cell (such as a cytotoxic cell), and b) instructions for administering the composition to an individual for the treatment of a disease associated with expression, aberrant expression and/or aberrant activity of WT1 (e.g., WT1-RMF) (e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)). In some embodiments, the association is by conjugation of the anti-WTMC construct or construct combination to a molecule on the surface of the cell. In some embodiments, the association is by insertion of a portion of the anti-WTMC construct or construct combination into the outer membrane of the cell.

In some embodiments, the kit comprises a nucleic acid (or set of nucleic acids) encoding an anti-WTMC construct (e.g., a full-length anti-WTMC antibody, a mono-specific anti-WTMC construct, a multispecific anti-WTMC construct, e.g., a bispecific anti-WTMC construct (such as a bispecific anti-WTMC antibody, e.g., anti-WTMC tandem di-scFv), an anti-WTMC CAR, an anti-WTMC immunoconjugate, or other anti-WTMC construct described herein), an anti-WTMC construct combination (e.g., an anti-WTMC caTCR+anti-WTMC, an anti-WTMC caTCR+anti-WTMC CSR, an anti-WTMC CAR+CSR, an anti-WTMC CAR+anti-WTMC CSR, an anti-WTMC CSR+CAR, an anti-WTMC CSR+anti-WTMC caTCR) described herein, or the polypeptide portion(s) thereof. In some embodiments, the kit comprises a) a nucleic acid (or set of nucleic acids) encoding an anti-WTMC construct (or construct combination) or polypeptide portion(s) thereof, and b) a host cell (such as an effector cell, e.g., a T cell) for expressing the nucleic acid (or set of nucleic acids). In some embodiments, the kit comprises a) a nucleic acid (or set of nucleic acids) encoding an anti-WTMC construct (or construct combination) or polypeptide portion(s) thereof, and b) instructions for i) expressing the anti-WTMC construct (or construct combination) in a host cell (such as an effector cell, e.g., a T cell), ii) preparing a composition comprising the anti-WTMC construct (or construct combination) or the host cell (e.g., effector cell, e.g., T cell) expressing the anti-WTMC construct (or construct combination), and iii) administering the composition comprising the anti-WTMC construct (or construct combination) or the host cell expressing the anti-WTMC construct (or construct combination) to an individual for the treatment of a disease associated with expression, aberrant expression and/or aberrant activity of WT1 (e.g., WT1-RMF) (e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MD), mesothelioma, ovarian cancer, gastrointestinal cancers, breast cancer, prostate cancer or glioblastoma)). In some embodiments, the host cell (e.g., effector cell, such as a T cell) is derived from the individual. In some embodiments, the kit comprises a) a nucleic acid (or set of nucleic acids) encoding an anti-WTMC construct (or construct combination) or polypeptide portion(s) thereof, b) a host cell (such as an effector cell, e.g., a T cell) for expressing the nucleic acid (or set of nucleic acids), and c) instructions for i) expressing the anti-WTMC construct (or construct combination) in the host cell, ii) preparing a composition comprising the anti-WTMC construct (or construct combination) or the host cell expressing the anti-WTMC construct (or construct combination), and iii) administering the composition comprising the anti-WTMC construct (or construct combination) or the host cell expressing the anti-WTMC construct (or construct combination) to an individual for the treatment of a disease associated with expression, aberrant expression and/or aberrant activity of WT1 (e.g., WT1-RMF) (e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)).

In some embodiments, the kit comprises a nucleic acid encoding an anti-WTMC CAR. In some embodiments, the kit comprises a vector comprising a nucleic acid encoding an anti-WTMC CAR. In some embodiments, the kit comprises a) a vector comprising a nucleic acid encoding an anti-WTMC CAR, and b) instructions for i) introducing the vector into effector cells, such as T cells derived from an individual, ii) preparing a composition comprising the anti-WTMC CAR effector cells, and iii) administering the anti-WTMC CAR effector cell composition to the individual for treatment of a disease associated with expression, aberrant expression and/or aberrant activity of WT1 (e.g., WT1-RMF) (e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)). In some embodiments, the kit further comprises a) vector(s) comprising a nucleic acid that encodes a bispecific construct, e.g., a tandem di-scFv (such as an anti-WTMC tandem scFv), and b) instructions for i) introducing the vector(s) encoding the tandem di-scFv into the host cell simultaneously or sequentially with the vector encoding the anti-WTMC CAR, ii) preparing a composition comprising the anti-WTMC CAR plus tandem di-scFv effector cells, and iii) administering the anti-WTMC CAR plus tandem di-scFv effector cell composition to the individual for treatment of a disease associated with expression, aberrant expression and/or aberrant activity of WT1 (e.g., WT1-RMF) (e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)). In some embodiments, the kit further comprises a) vector(s) comprising a nucleic acid that encodes a CSR (such as an anti-WTMC CSR), and b) instructions for i) introducing the vector(s) encoding the CSR into the host cell simultaneously or sequentially with the vector encoding the anti-WTMC CAR, ii) preparing a composition comprising the anti-WTMC caTCR plus CSR effector cells, and iii) administering the anti-WTMC CAR plus CSR effector cell composition to the individual for treatment of a disease associated with expression, aberrant expression and/or aberrant activity of WT1 (e.g., WT1-RMF) (e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)).

In some embodiments, the kit comprises a nucleic acid encoding an anti-WTMC caTCR. In some embodiments, the kit comprises a vector comprising a nucleic acid encoding an anti-anti-WTMC caTCR. In some embodiments, the kit further comprises nucleic acid(s) encoding a bispecific construct, e.g., a tandem di-scFv (e.g., an anti-WTMC tandem di-scFv) or a CSR (such as an anti-WTMC CSR). In some embodiments, the kit comprises a) a vector comprising a nucleic acid encoding an anti-WTMC caTCR, and b) instructions for i) introducing the vector into effector cells, such as T cells derived from an individual, ii) preparing a composition comprising the anti-WTMC caTCR effector cells, and iii) administering the anti-WTMC caTCR effector cell composition to the individual for treatment of a disease associated with expression, aberrant expression and/or aberrant activity of WT1 (e.g., WT1-RMF) (e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)). In some embodiments, the kit further comprises a) vector(s) comprising a nucleic acid that encodes a bispecific construct, e.g., a tandem di-scFv (such as an anti-WTMC tandem scFv), and b) instructions for i) introducing the vector(s) encoding the tandem di-scFv into the host cell simultaneously or sequentially with the vector encoding the anti-WTMC caTCR, ii) preparing a composition comprising the anti-WTMC caTCR plus tandem di-scFv effector cells, and iii) administering the anti-WTMC caTCR plus tandem di-scFv effector cell composition to the individual for treatment of a disease associated with expression, aberrant expression and/or aberrant activity of WT1 (e.g., WT1-RMF) (e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)). In some embodiments, the kit further comprises a) vector(s) comprising a nucleic acid that encodes a CSR (such as an anti-WTMC CSR), and b) instructions for i) introducing the vector(s) encoding the CSR into the host cell simultaneously or sequentially with the vector encoding the anti-WTMC caTCR, ii) preparing a composition comprising the anti-WTMC caTCR plus CSR effector cells, and iii) administering the anti-WTMC caTCR plus CSR effector cell composition to the individual for treatment of a disease associated with expression, aberrant expression and/or aberrant activity of WT1 (e.g., WT1-RMF) (e.g., WT1-positive cancer (such as chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma)).

The kits described herein are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of compositions comprising an anti-WTMC construct (or construct combination) generally include information as to dosage, dosing schedule, and route of administration for the intended treatment. The containers may be unit doses, bulk packages (e.g., multi-dose packages) or sub-unit doses. For example, kits may be provided that contain sufficient dosages of an anti-WTMC construct (e.g., a full-length anti-WTMC antibody, a multispecific anti-WTMC molecule (such as a bispecific anti-WTMC antibody), an anti-WTMC CAR, an anti-WTMC caTCR, an anti-WTMC CSR, an anti-WTMC immunoconjugate, or other anti-WTMC construct or construct combination described herein) to provide effective treatment of an individual for an extended period, such as any of a week, 8 days, 9 days, 10 days, 11 days, 12 days, 13 days, 2 weeks, 3 weeks, 4 weeks, 6 weeks, 8 weeks, 3 months, 4 months, 5 months, 7 months, 8 months, 9 months, or more. Kits may also include multiple unit doses of the anti-WTMC construct (or construct combination) and pharmaceutical compositions and instructions for use and packaged in quantities sufficient for storage and use in pharmacies, for example, hospital pharmacies and compounding pharmacies.

### EXAMPLES

Those skilled in the art will recognize that several embodiments are possible within the scope and spirit of this present application. The present application will now be described in greater detail by reference to the following non-limiting examples. The following examples further illustrate the present application but, of course, should not be construed as in any way limiting its scope.

### Example 1: Experimental Materials and Methods

### Cell Samples, Cell Lines, Peptides and Antibodies

After informed consent on Institutional Review Board-approved protocols, PBMCs from HLA-typed healthy donors and patients were obtained by Ficoll density centrifugation. Cell lines were maintained at MSKCC and were originally obtained from ATCC. The following cells were gifts from the listed labs at MSKCC: AML14 cell line; OCI-AML02 and AML patient-derived xenograft (PDXs). Non-hematopoietic cells were purchased from Science Cell Research laboratories (Carlsbad, CA). Cells were cultured in the medium with respective supplements provided by the vendor, according to the instruction of the manufacturer. All the peptides used in this study were synthesized by and purchased from Genemed Synthesis, Inc (San Antonio, TX) and the purity of the peptides are greater than 95%.

Monoclonal antibodies against human HLA-A2 (clone BB7.2) conjugated to fluorescein isothiocyanate (FITC) or allophycocyanin (APC), and its isotype control mouse IgG2b/FITC or APC, to human CD3, CD15, CD19, CD33, CD34, (goat F(ab′)₂ anti-hIgG conjugated with phycoerythrin (PE) or FITC, and goat F(ab′)₂ anti-mouse Ig conjugated to FITC were purchased from BioLegend (San Diego, CA). Human isotype control hIgG1 antibody was provided by Eureka Therapeutics (catalog number ET901). ESK1 mouse IgG1, ESK1-BiTE and its control were produced by Eureka Therapeutics (Emeryville, CA). The CFSE labeling kit was purchased from Thermo Fisher (Waltham, MA). The MACSxpress® Human Whole Blood Neutrophil Isolation Kit was purchased from Miltenyi Biotech (San Diego, CA).

### Animals

Seven- to 10-week-old male NOD.Cg-Prkdc SCID IL2rgtm1Wjl/SzJ mice, known as NSG, were purchased from The Jackson Laboratory and hosted in pathogen-free MSKCC animal facility.

### Flow Cytometry Analysis

For cell surface staining, cells were incubated with appropriate mAbs for 30 min on ice, washed, and incubated with secondary antibody reagents when necessary. Flow cytometry data were collected on a LSRFortessa™ Cell Analyzer (BD) and analyzed with FlowJo™ v10 software (FlowJo LLC, Ashland, OR).

Selection and Characterization of scFv Specific for WT1 RMF Peptide/HLA-A2 Complexes

Biologically active antibody constructs were selected from Eureka Therapeutics' E-ALPHA© phage library, a collection of human scFv antibody phage display libraries containing over 10×10¹⁰ unique clones. The phage libraries included naïve libraries comprising fully naïve human heavy and light chain repertoires. The E-ALPHA library was used to screen for the selection of human antibody constructs (e.g., scFv) specific for the human WT1 RMF/HLA-A2 complex.

To select phage clones that mimic TCR recognition, i.e., recognizing amino acids in the middle of the peptide/HLA complex, the following screening strategies were performed: first, counter screening against a mixture of 20 irrelevant HLA-A2-binding peptide complex to remove all clones that bound to the HLA-A2 molecule. Second: positive screening for WT1 RMF (RMFPNAPYL) (SEQ ID NO: 113)/ILA-A2 complex to broadly select clones that bound to the desired RMF/A2 complex. Third: screening against a mutant peptide “RMF-AAA” (RMAAADPYL) (SEQ ID NO: 114)/HLA-A2 complex to remove phage clones that bind to either end of the target peptide. Specificity to the middle amino acids FPNA (SEQ ID NO: 349) should reduce binding to many potential human proteomic off-target peptides. The selective binding of phage clones was validated by standard ELISA method against biotinylated single chain RMF/HLA-A2 peptide complexes.

Binding to peptide/HLA-A2 complexes on live cell surfaces was determined using a TAP-deficient, HLA-A2+ cell line, T2, as previously described (11, 37). Fourteen anti-WT1 RMF/HLA-A2 clones showed robust and specific binding to T2 cells pulsed with RMF peptide, but not to T2 cells left unpulsed or pulsed with the irrelevant control HLA-A2-binding peptide. The 14 clones were selected and used to determine the specificity in more detailed analyses which included using purified phage clones, hIgG1, or the BiTE format to test the 1) binding and killing against T2 cells pulsed with RMF, and other potentially cross-reactive peptides identified by previous studies with the ESK1 mAb (Table 1-peptides); 2) alanine substitution of each residue of the peptide to identify the binding positions on T2 cells. 3) binding to and killing against PBMCs and whole blood from HLA-A2 positive or negative healthy donor and WT1+/HLA-A2+ or negative tumor cell lines.

The 14 clones with unique sequences (the ESK2 antibody clones) were chosen based on their selective profiles and characterization in the above assays, and their antigen-recognition domains were engineered into caTCR constructs.

Generation of mIgG1 and BiTE Forms of ESK2-Clones

ESK2 BiTEs were produced as described by Yeku et al (38). In brief, the BiTE consists of one ESK2 scFv at the N terminus, and the other arm of an anti-CD3E scFv (derived from mouse monoclonal antibody L2K) at the C terminus. The two scFv are joined covalently through a linker GGGGSGGGGSGGGGS (SEQ ID NO: 258) and cloned into mammalian expression vectors. A His tag was inserted at the C-terminal end for purification after BiTE production in CHO cells.

Full-length human IgG1 of the selected ESK2 phage clones were produced in HEK293 and Chinese hamster ovary (CHO) cell lines, as described. In brief, antibody variable regions were subcloned into mammalian expression vectors, with matching Lambda or Kappa light chain constant sequences and IgG1 subclass Fc.

Determination of affinity of ESK2 clones

Multiple cycles of antibody binding kinetics were measured by Biacore™ (Cytiva) with a biotin CAP sensor chip by following the manufacturer's protocol. The biotinylated WT1 RMF pMHC complex (10 μg/ml) was loaded onto CAP sensor chip first at 5 ul/min for 120 seconds. Then, the ESK2-BiTE clones #18 and #34 were injected over the sensor chip at 150, 75, 37.5, 18.75, 9.375, 4.6875 and 0 nM, (independently), with a flow rate of 30 ul/min. The association time and the disassociation time were 180 and 300 seconds, respectively. The kinetic analysis was performed by the Biacore™ X100 evaluation software using a 1:1 binding model.

Generation of ESK-2 caTCR Constructs of the Present Technology and Characterization

Nucleic acids from 14 ESK2 clones were engineered to encode ESK-2 caTCR alone (the ESK2 caTCR constructs) or encode both ESK-2 caTCR and anti-CD33 costimulatory receptor (CSR) constructs (ESK2 2.0). First, 14 ESK2 antibody clones binding to WT1 RMF/HLA-A*02:01 were selected as described in the previous paragraph, and their antigen-recognition domains in the format of Fabs were engineered into caTCR constructs, named ESK2 caTCRs. The TCR component of caTCR (composed of partial constant region and the entire transmembrane and intracellular regions of a TCR) is derived from the human TCR γ (UniProtKB: locus TRGC1_HUMAN, accession POCF51) and 6 (UniProtKB: locus TRDC_HUMAN, accession B7Z8K6) chains.

Vectors encoding the 14 ESK2 caTCR constructs were used to transduce primary T cells. The transduced T cells were tested for their in vitro cytotoxicity and specificity by co-culturing with T2 cells loaded with RMF or other control peptides (CytoTox 96 non-radioactive kit, Promega). Eight of the 14 different ESK2 caTCR T cells were selected based on their cytotoxicity against RMF loaded T2 cells and low cross reactivity with other peptides. These eight ESK2 caTCR T cells were further characterized using stable target cell lines that were generated by ectopically expressing the RMF peptide region (RMF minigene, MG) in HLA-A*02:01-positive cell lines SK-Hep1. Finally, the specificity was tested using the AML cell lines SKM-1 and SET-2 which are WT1 and HLA-A2 positive. Expression of costimulatory ligands on AML cell lines were stained with antibodies (anti-hCD80 antibody clone 2D10, anti-hCD86 antibody clone IT2.2, anti-hCD137L clone 5F4, Biolegend) and determined by flow cytometric analysis.

Two of the eight ESK2 caTCRs, which were derived from ESK2 antibody clones #18 and #34, were selected for generation of T cells comprising ESK-2 caTCR constructs of the present technology which co-express ESK2 caTCR and anti-CD33 costimulatory receptor (CSR). The CSR is composed of an anti-CD33 scFv linked to a partial CD28 molecule (consisting of the hinge, transmembrane and intracellular domains of CD28) and includes a Myc tag.

The DNA sequences encoding the ESK2 constructs (ESK2 caTCR+anti-CD33 CSR) were cloned into a pCDH lentiviral vector (Systems Biosciences) for delivery into primary T cells.

Transduction of ESK-2 caTCR Constructs of the Present Technology

T cells were isolated using negative selection and then stimulated with CD3/CD28 Dynabeads™ (Thermo Fisher Scientific). After 24 hours, activated T cells were transduced with lentivirus at an MOI of 2-5 and cultured in supplemented RPMI 1640 medium. Transduction efficiency for T cells positive for ESK-2 caTCR constructs of the present technology was determined by flow cytometry using an anti-Myc antibody (Cell Signaling Technology, Danvers, MA) or an anti-F (ab′)₂ antibody (Jackson Immuno Research, West Grove, PA). Mock T cells are control T cells from the same donors treated with the same procedure to make AbTCR-CSR T cells, but without transduction of the AbTCR or CSR constructs. The percentage of receptor positive T cells was normalized with donor-matched mock T cells (un-transduced) for all experiments.

### Short-Term Killing Using a Cytokine Release Assay

The short-term killing ability of the various caTCR T cells was determined by measuring the amounts/levels of cytokines released from T cells upon engagement with target cells. T cells with high cytotoxic potency secrete high levels of cytokines related to T cell activity, such as IFN-γ and IL-2. Mock (i.e., mock T cells that do not express a caTCR that targets WT1) and ESK2 caTCR-transduced T cells were incubated for 16 hours at a 1:1 effector cell to target cell (E:T) ratio in a 200 μl volume. Evaluation of cytokine production by T cells expressing caTCR clones following in vitro stimulation was conducted by flow cytometric analysis of intracellular IFN-7. Cytokine release was quantified with Luminex Magpix® technology using the Bio-Plex Pro™ Human Cytokine 8-plex assay (Bio-Rad) according to the manufacturer's instructions.

### Short-Term Killing Using an LDH Release Assay

To measure the ability of the ESK2 caTCR clone-expressing T cells of the present disclosure to kill cancer/tumor cells, a FACS-based assay comparing the short-term killing ability of T cells expressing ESK2 clones was performed. Activated effector cells (caTCR-expressing cells) and their corresponding target cells (tumor cell lines expressing the RMF minigene, HepG2-WT1 MG and SK-Hep1-WT1 MG) were co-cultured at an effector to target (E:T) ratio of 1:1 for 16 hours in a 200 μl volume. To measure membrane integrity (lysis) in cell-mediated cytotoxicity experiments, an LDH release assay was employed. Specific killing was determined using a CytoTox 96® Non-Radioactive Cytotoxicity Assay (Promega, Madison, WI) to measure LDH released into cell culture supernatants. Visible wavelength (490 nm) absorbance data were collected using a standard 96-well plate reader. Untransduced (Mock) T cells served as controls. K562 cells are HLA-A2 negative, HepG2 and SKHep1 cells are WT1 negative and SET-2 and SKM-1 cells are WT1-positive and HLA-A*02:01-positive. Percent cytotoxicity was calculated using the following formula: % Cytotoxicity=Experimental-Effector spontaneous-Target spontaneous/Target maximum-Target spontaneous×100.

Cytotoxicity Assays of T Cells Comprising ESK-2 caTCR Constructs of the Present Technology

Cytotoxicity of T cells comprising ESK-2 caTCR constructs of the present technology against target cells was measured by luciferase-based assay or FACS-based killing. In brief, T cells comprising ESK-2 caTCR constructs of the present technology and target cells were incubated at a 1:1 E:T ratio in 200 μl volume for 16 hours.

For the luciferase-based assay, target cells expressing firefly luciferase and GFP (fLuc-GFP) were used. Effector and tumor target cells were co-cultured in triplicate at the indicated E:T ratio using clear bottom, white 96-well assay plates (Corning 3903) with 1×104 target cells in a total volume of 200 μl. Target cells alone were plated at the same cell density to determine maximum luciferase activity. Cells were co-cultured overnight, at which time d-luciferin substrate (Gold Biotech LUCK) was added at a final concentration of 0.5 μg/μl to each well. Emitted light was detected in a Wallac EnVision Multilabel reader (Perkin Elmer). Target lysis was determined as (1−(RLUsample)/(RLUmax))×100 (RLU, relative light unit).

For the flow cytometry-based assay, target cells were labeled with CFSE and co-incubated with T cells comprising ESK-2 caTCR constructs of the present technology or mock-T cells (untransduced) at E: T ratio of 1:1 overnight. The cells were harvested, washed and stained with mAbs for CD33 and other AML markers and zombie (dead cells) and subjected to flow cytometry. The percentage reduction of CFSE positive cells or CFSE and Zombie double positive cells over CFSE positive cells were determined as % death of the targets.

### Colony Forming Unit (CFU) Assay

10⁴ CD34+ cells were cocultured with 10⁴ control Mock T or AbTCR-CSR T cells (clone #18 and #34) cells for 16 hours, and were plated (in triplicates) in methylcellulose (MethoCult™ H4₄₃₄ Classic—Stem Cell Technologies). CFU colonies were scored 4-14 days after seeding. For AML14 cells, two E:T ratios were performed at 1:1 and 1:10. For the 1:1:10⁴ AML14 cells were cocultured with 10⁴ control or AbTCR-CSR T cells; for 1:10, 105 AML14 cells were cocultured with 10⁴ control or CAR T cells. Cells were cocultured for 16 hrs. For CFU seeding, 10⁴AML14 cells were seeded in methylcellulose (MethoCult™ H4434 Classic—Stem Cell Technologies). CFU were scored 4-14 days after seeding.

### AbTCR-CSR Trans Cytotoxicity Assay

AbTCR-CSR cells (Mock T Cells, Clone #18, and Clone #34) were incubated at an E:T ratio of 1:1 overnight using a 6-well tissue culture-treated plate (Corning 3516). 4×105 of each cell were plated. Two different WT1 positive adherent targets (JMN and MSTO), HLA-A2+ and HLA-A2−, respectively, were co-cultured with each effector cell. The suspension effector cells were collected through aspiration and plated with AML-14. HL-60, target cells at 1:1 and 1:3 E:T ratios triplicates in a white 96-well assay plate. 104 target cells were plated with effector cells in a total of 200 L. After an overnight coculture, the cytotoxicity was measured through a luciferase assay reading.

Therapeutic Trials of T Cells Comprising ESK-2 caTCR Constructs of the Present Technology in AML Xenograft Animal Models

All animal studies were conducted on IACUC approved protocols. GFP/luciferase-transduced AML-14 cells (5 million) were injected intravenously into NSG mice. On day 13, tumor engraftment was confirmed by luciferase imaging and randomized to 4 groups. T cells comprising ESK-2 caTCR constructs of the present technology or mock-T cells (6 million) were injected intravenously, and tumor growth was monitored by bioluminescence imaging (BLI) every 5-7 days and survival of mice was documented. For a second AML model, OCI-AML-2 cells (0.5 million) were injected intravenously into NSG mice. Groups were blindly assigned to either treatment group and T cells comprising ESK-2 caTCR constructs of the present technology (one million) were iv injected on day 4 and day 12 post tumor cell injection. Tumor burden was assessed by BLI on the indicated days and the health and survival of mice was documented. The Mantal-Cox test was used to calculate the statistics of survival.

### Statistics

The p Value of tumor reduction by BLI among treatment groups in animal was determined by 2 way ANOVA test or unpaired t student test. Animal survival curves were evaluated by a Mantel-Cox test for the significance of differences between treatment groups.

### Example 2: TCRm ESK2 is Selective for WT1 RMF

Development of ESK2 was built on previous findings with the first TCRm (ESK1) against the same WT1 RMF/HLA-A2 complex. ESK1 binding to the peptide/MHC was positioned so that the CDR3 loops were focused over the α2 helix of the HLA, resulting in predominant reactivity to the N-terminal of the peptide to R in position 1 and to a lesser degree, to P in position 4 (20, 21). This positioning reduced specificity due to promiscuity of sequence variations on the C terminal. Therefore, screening strategies for ESK2 development were designed to obtain TCRm mAbs that recognized broader and more central amino acid residues of the RMF peptide. The screening strategy included: 1) positive phage display screening for binding to the RMF/HLA-A2 complex using the native RMF sequence (RMFPNAPYL; SEQ ID NO: 113); 2) negative screening to remove all scFv clones that bound to either end of the peptide using the peptide RMAAADPYL (SEQ ID NO: 114) peptide. Computational analyses (Table 1) suggested that specificity to the central FPNA (SEQ ID NO: 349) amino acids and not the terminal amino acids should remove nearly all off-target 9-mer peptides identified in exosome screening with predicted affinity below 500 nM to HLA-A2 molecules.

TABLE 1
Homolog peptides identified in human exome by in silico screening.

| Sequences tested for | | Sequences | | HLA-A2 |
|---|---|---|---|---|
| cross reactivity to | | found | | binding |
| RMFPNAPYL* | Epitope | in exome | | (Kd |
| (SEQ ID NO: 113) | name | (<1 uM Kd) | Source protein | prediction) |
| X(LMV)FPNAPY(LVI) | WT1- | RMFPNAPYL | Wilm's tumor 1 | 7 nm |
| (SEQ ID NO: 334) | RMF | (SEQ ID NO: | | |
| | | 113) | | |
| X(LMV)FPNAPY(LVI) | ABHD3- | SLYPSAPFL | Phospholipase | 7 nm |
| (SEQ ID NO: 334) | 9-mer | (SEQ ID NO: | ABHD3 | |
| and substitute any | | 330) | | |
| other position with | | | | |
| any conserved aa | | | | |
| Same as above but | ABHD3- | SLYPSAPFLA | Phospholipase | 14 nm |
| with 10 residues | 10-mer | (SEQ ID NO: | ABHD3 | |
| instead | | 331) | | |
| | AMFR | EMFPQVPYHL | Autocrine | 148 nm |
| | | (SEQ ID NO: | Motility Factor | |
| | | 332) | Receptor | |
| Identified | TSTP1- | RLFPNAKFLL | Tyrosyl protein | 27 nM |
| previously (Ref 20) | 10-mer | (SEQ ID NO: | sulfotransferase 1 | |
| | | 333) | | |

Potential off-target sequences were identified using ScanProsite (Expasy) to search the human exome (UniProtKB/Swiss-Prot) for homologous 9 amino acid peptides that had predicted affinity to HLA-A*02:01 (HLA-A2) of less than 500 nM using NetMHCPan. *The rules used to identify off-target sequences are as follows:
Position #1 could be any amino acid “X”; position #2 and #9 could be any canonical anchor (LMV) and (LVI). Position #3-#8 could be replaced with any conserved residues shown below at their respective positions in RMFPNAPYL (SEQ ID NO: 113): F=>Y=W, P=>P; N>S=T=Q; A=>V=I=G; Y=>F=W. Additional homologous 10-mers were also included. TSTP1 was identified in a previous study (20)

Selected phage clones were converted to the BiTE format for screening of binding to cell targets and cytotoxicity assays. A mouse IgG1 format was used for the flow cytometric assays.

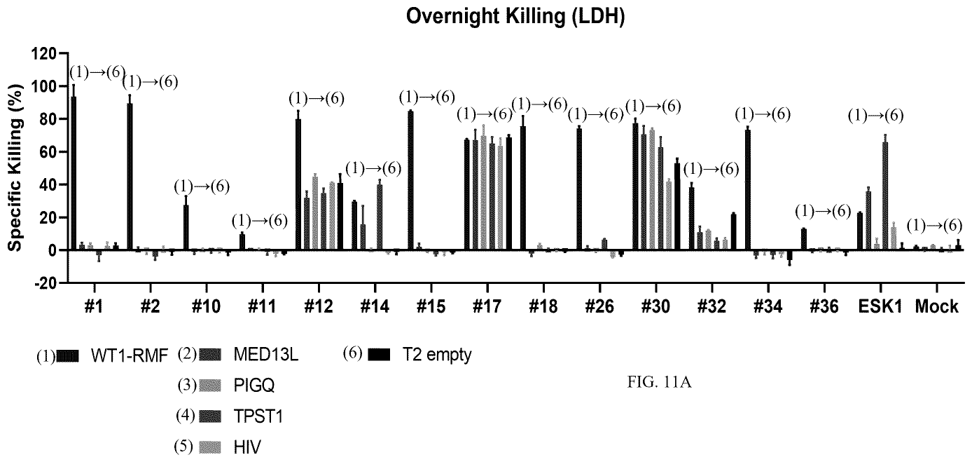

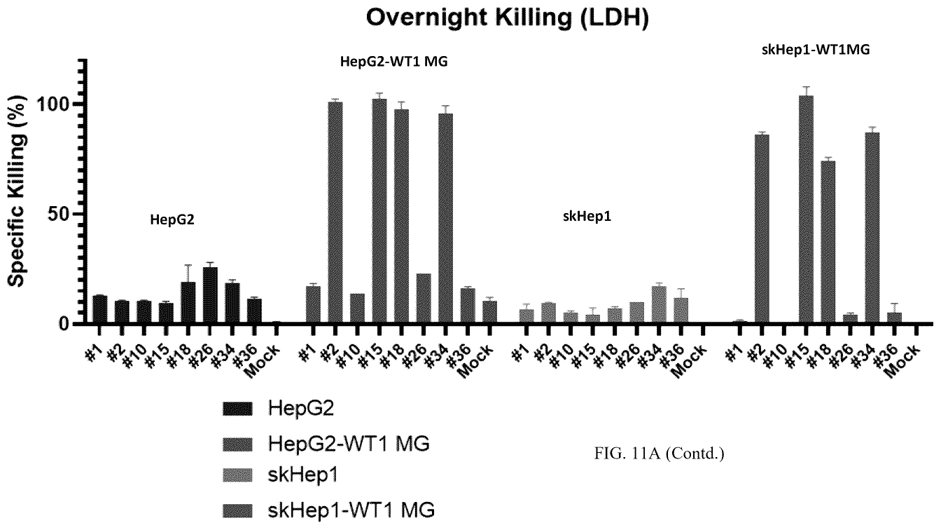

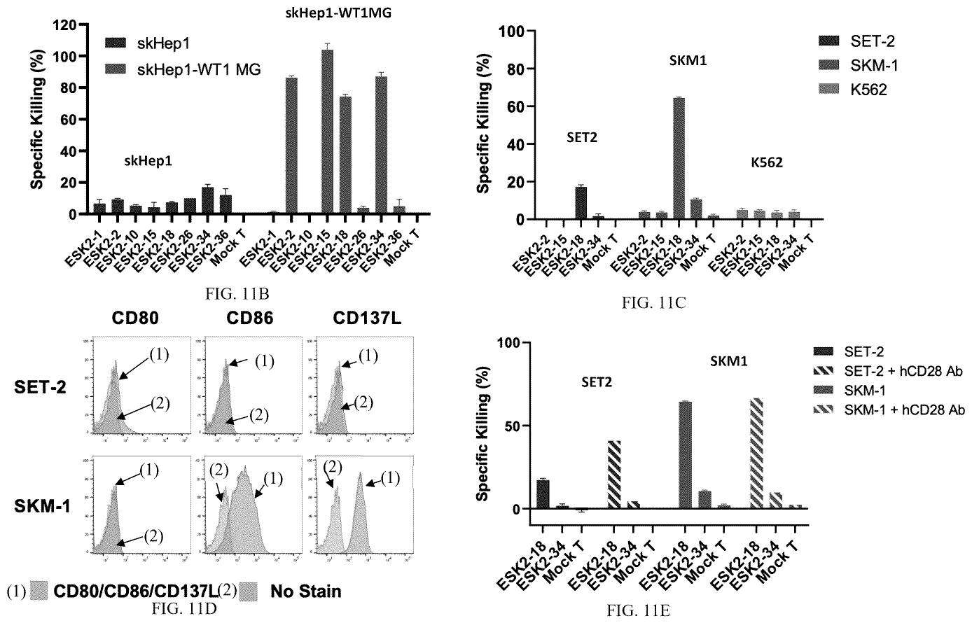

FIG. 11 shows the identification and characterization of caTCR specific for WT1 RMF/HLA-A*02:01. Fourteen ESK2 caTCR clones were introduced into primary T cells and tested for in vitro cytotoxicity and specificity in a 16-hour coculture with target cells. Eight ESK2 caTCR T cells were further screened for cytotoxicity by co-culture with HEPG2, HEPG2-WT1 MG (MG cell line ectopically expressing RMF peptide region (RMF mini-gene, MG)), SK-Hep1, and an artificial SK-Hep1-WT1 MG cell line ectopically expressing RMF peptide region (RMF mini-gene, MG) in HLA-A*02:01-positive SK-HEP1.

ESK2 clones #18 and #34 were selected for detailed characterization based on their on- and off-target profiles and the cytotoxicity in IgG and BiTE formats. The specificity of ESK2-clone #18 and #34 on T2 cells pulsed with RMF, mutant peptides, and exomic peptides that represent potential off-targets were tested (Table 1). Both clones showed stronger binding to T2 cells pulsed with RMF peptide than ESK1, but not to T2 alone or pulsed with an irrelevant HLA-A2 binding peptide derived from HPV-E7. While ESK1 bound WT1-RMAAA mutant peptide, and off-target peptides TSTP1, clone #18 did not bind to any of these peptides but clone #34 bound to the ABHD3-9mer peptide (FIG. 1A).

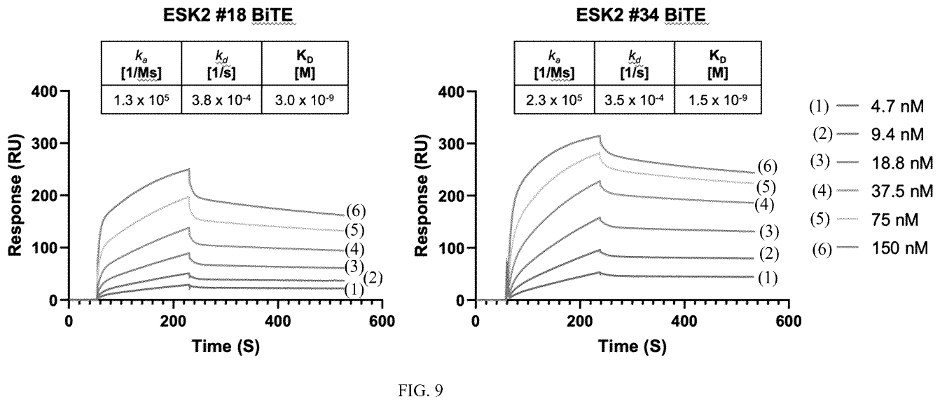

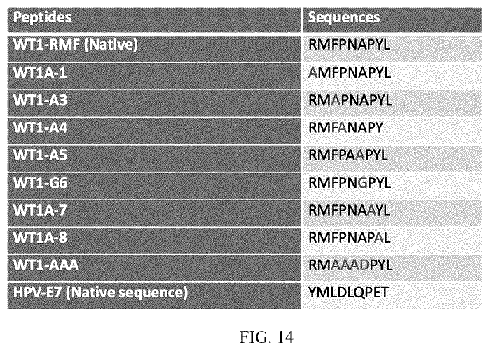

The lack of the binding to a particular peptide was not due to impaired peptide pulsing onto HLA-A2 because all the peptides showed reasonable HLA-A2 stabilization in T2 cells (FIG. 1B). Differential binding profiles of ESK2 clones compared to ESK1 were demonstrated by alanine screening on T2 cells (FIG. 14). ESK1 recognition largely depended on position 1, 4 and 6 of the RMF peptide (11, 20, 21). In contrast, the key amino acids required for binding of clone #18 had a wide range from position 1, 3, 7 and 8 (R, F, P, Y). Binding of #34 to the RMF peptide were residues P, and Y, at positions, 7 and 8 respectively. The Y seemed to be particularly important. The A4P and A6G substitutions also reduced the HLA-A2 expression, confounding interpretation of results at these positions (FIGS. 1C-1D). Mutations on anchor residues in positions 2 and 9 would reduce the peptide binding and therefore they were left intact. These results demonstrated that clone #18 recognized a broader and more centrally located group of amino acids in the RMF/HLA-A2 complex. Clone #34 recognition was more C-terminally oriented, although reduced recognition of positions 4 and 6 was more profound than the reduction of HLA-A2 expression. Biacore™ analysis showed that clone #18 and #34 BiTEs have comparable affinities of 3 nM and 1.5 nM, respectively (FIG. 9).

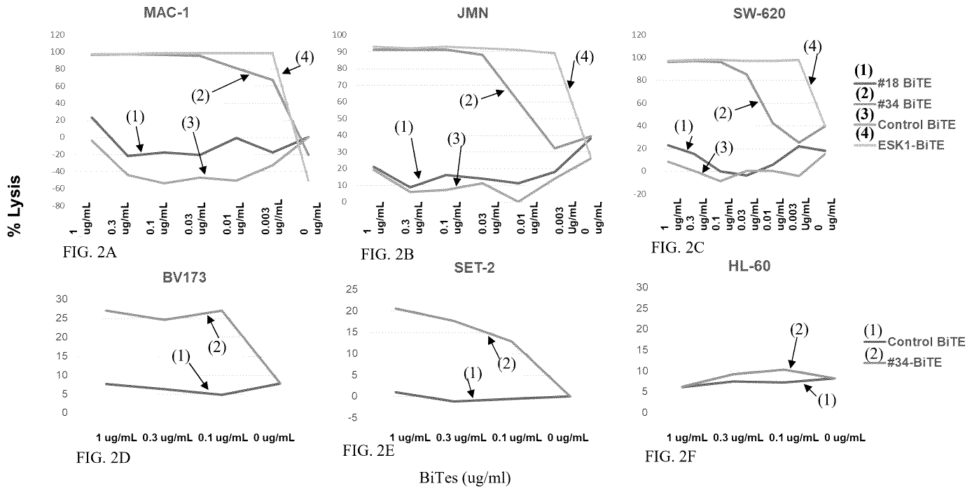

The specificity and cytotoxicity of ESK2-BiTEs was tested against a panel of tumor cell lines. While clone #34 could bind to and kill various leukemia and solid tumor cells, no binding or killing was observed with HLA-A2(−)/WT1(+) cell line HL-60 nor with HLA-A2(+)/WT1(−) cell line SKLY-16, showing that the activity of clone #34 was both WT1 and HLA-A2-dependent (Table 2, FIGS. 2A, 2B and 2C).

TABLE 2
Tumor cell lines used for ESK2 clone
#34 binding and cytotoxicity.

| Tumor | Tumor | RMF/HLA- | | |
|---|---|---|---|---|
| Cell | Tissue | A2 complex | HLA-A2 | Cytotoxicity |
| Name | Type | Expression | Expression | (using BiTE) |
| AML-14 | AML | + | +++ | +++ |
| BV173 | Chronic | + | ++ | ++ |
| | myeloid | | | |
| | leukemia | | | |
| | (B-blastic) | | | |
| SET-2 | AML | + | ++ | ++ |
| OCI-AML-2 | AML | + | ++ | NT |
| JMN | Mesothelioma | + | ++ | ++ |
| MAC-1 | T cell | ++ | +++ | +++ |
| | lymphoma | | | |
| MAC-2A | T cell | ++ | +++ | +++ |
| | lymphoma | | | |
| OV56 | Ovarian cancer | + | ++ | ++ |
| SW-620 | Colon cancer | + | ++ | ++ |
| HL-60 | AML | − | − | − |
| SKLY-16 | NHL | − | + | − |

The presentation of WT1 RMF/HLA-A2 complex is measured by binding to tumor cells using mouse ESK2 #34 (3 μg/ml). Binding of the ESK2 was ranked as fold increase over isotype control: “+” (1.5 to 3-fold increases); “++” (>5-10 fold); “−” (indicates no binding at all). HLA-A2 expression was measured by mouse anti-HLA-A2 mAb, clone BB7. “+++” (>80-fold increases over isotype control); “++” (40 to 80 fold); + (2-10-fold). Cytotoxicity was tested by ESK2 #34 BiTE at various concentrations (examples shown in FIG. 2). Cytotoxicity mediated by ESK2-#34 BiTE was measured by either standard ⁵¹Cr-release (5 hours) or luciferase assay (overnight). The killing activity depends on the dosage and the incubation duration as shown in FIG. 2. “++” indicates 3 to 5 fold-increases in the killing over isotype control; “+++” indicates >10 fold killing. AML: Acute myeloid leukemia; CML: Chronic Myeloid Leukemia; NHL: Non-Hodgkin's Lymphoma. WT1 expression of the cell lines use here were tested for their WT mRNA or protein by us and others in previous studies (Ref. 9, 10, 13, 16). The data are summarized from 3-7 experiments, depending on cell lines.

Unexpectedly, ESK2-#18 BiTE did not show any detectable cytotoxicity against the same tumor cells, tested in parallel, in an overnight coincubation (FIGS. 2A, 2B and 2C). The specific cytolytic activity of clone #34 was further confirmed in 5 hr-⁵¹Cr lysis assay (FIGS. 2D, 2E and 2F). Overnight cultures showed greater cytolytic activity. ESK1 BiTE showed slightly stronger cytotoxicity against the same target cells than ESK2-#34 BiTE, which could be due to a higher affinity of ESK1 than ESK2.

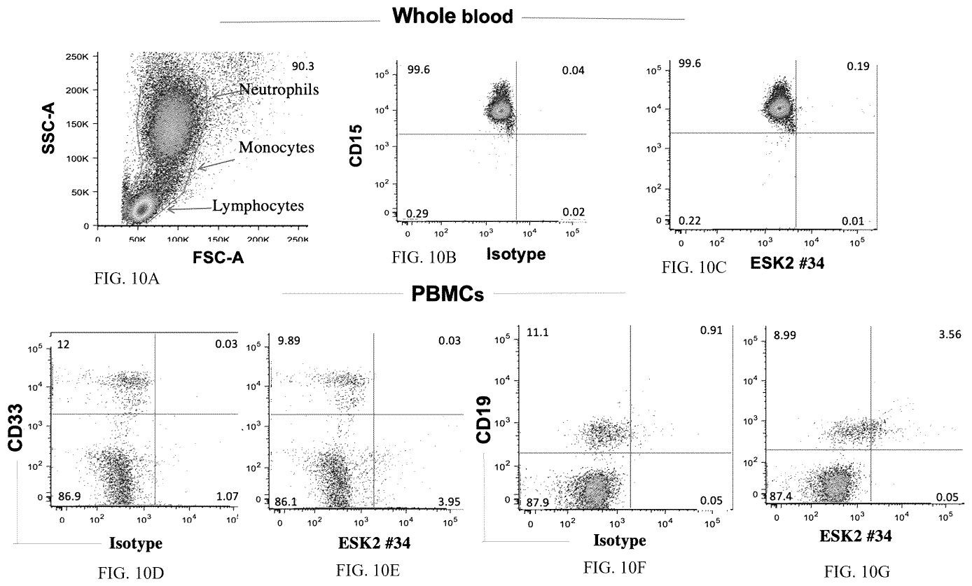

Importantly, no significant binding nor cytotoxicity of clone #34 was seen against whole blood including CD15+ neutrophils or normal CD3 T cells and CD33+ monocytes of PBMC from healthy HLA-A2 positive or negative donors (Table 3 and FIGS. 10A-10E).

TABLE 3
Summary of binding and cytotoxicity of ESK2 #34 on a panel
of normal hematopoietic cells and non-hematopoietic cells.
Hematopoietic Cells from Healthy Donors

| Whole blood (Binding/Cytotoxicity) | | | PBMCs (Binding/Cytotoxicity) | | |
|---|---|---|---|---|---|
| | HLA-A2+ | HLA-A2− | | HLA-A2+ | HLA-A2− |
| Neutrophils | Neg/Neg | Neg/Neg | T cells | Neg/Neg | Neg/Neg |
| (CD15+) | | | (CD3+) | | |
| Monocytes | Neg/Neg | Neg/Neg | Monocytes | Neg/Neg | Neg/Neg |
| (CD33+) | | | (CD33+) | | |
| Lymphocytes | 4%+/Neg | Neg/Neg | Monocytes | Neg/Neg | Neg/Neg |
| (CD45+) | | | (CD14+) | | |
| | | | B cells | 2-3%/Neg | Neg/Neg |
| | | | (CD19+) | | |

Non-Hematopoietic Normal Cells

| | WT1 mRNA | HLA-A2 | | |
|---|---|---|---|---|
| | expression | expression | Binding | Cytotoxicity |
| Adrenal | +++ | Neg | Neg | Neg |
| microvascular | | | | |
| endothelial cells | | | | |
| Renal glomerular | +++ | Pos | Neg | Neg |
| endothelial cells | | | | |
| Cardiac myocytes | NT | Neg | Neg | Neg |
| Testicular | NT | Pos | Neg | Neg |
| endothelial cells | | | | |

WT1 mRNA expression was measured by RT-PCR, described in ref. 16. Binding to normal cells was determined by ESK2 #34 mouse IgG1 (3 μg/ml) over control isotype staining. Cytotoxicity was measured by ESK2 #34 BiTE at concentration of 0.1, 1, or 10 μg/ml in standard ⁵¹Cr-release assay as described in the Materials and Methods. “+++” indicates 10⁴ to 10⁵ increases of WT1 transcript over negative control cells. NT = not tested. Pos = >20-fold increase over isotype control staining for HLA-A2 (BB7.2 clone). Neg = No increase over isotype control staining. For hematopoietic cells, the data are representative of six donors.

There was a minimal binding to CD19+B cells over isotype control (FIGS. 10F-10G). However, when the clone #34 BiTE was tested at concentrations of 10, 1 or 0.1 μg/ml, no cytotoxicity against T, B or monocytes/macrophages in PBMCs, or neutrophils in whole blood from healthy HLA-A2 positive donors was seen. Furthermore, clone #34 binding and cytolytic activity was tested against normal adrenal microvascular endothelial tissue, renal glomerular endothelial tissue, cardiac myocytes and testicular endothelial cells. There was no binding or killing by clone #34 against any of these cells, regardless of HLA-A2 expression (Table 3).

ESK2 clones were also tested in the caTCR format as candidates for T cell therapy against AML. In brief, 14 candidate ESK2 TCRm clones were adopted to the caTCR platform. The clones were introduced into primary T cells and the in vitro cytotoxicity and specificity were tested by co-culture with T2 cells loaded with RMF or other control peptides (FIG. 11A). After a 16-hour co-culture, T cells expressing one of eight different caTCRs were selected for substantial cytotoxicity against RMF loaded T2 cells and low cross reactivity with other peptides.

The eight different caTCR T cells were further screened for cytotoxicity against an artificial cell line SK-Hep1-WT1 MG which was generated by ectopically expressing RMF peptide region (RMF mini-gene, MG) in the HLA-A*02:01-positive SK-Hep1 parental cell line. caTCR T cells of clones #2, #15, #18 and #34 stood out for significantly higher killing activity compared with other clones after 16-hour co-culture with SK-Hep1-WT1 MG (FIG. 11B).

Next, the four leading caTCR T cell candidates were tested by co-culture with natural AML cell lines SET-2 and SKM-1 (HLA-A*02:01+/WT1+) (FIG. 11C). caTCR T cells of clones #18 and #34 showed specific killing activity, especially against SKM-1, hence selected as the final leading ESK2 caTCR T cell clones. Meanwhile, neither clone showed cytotoxicity against control cell line K562 which is WT1-positive and HLA-A*02:01-negative.

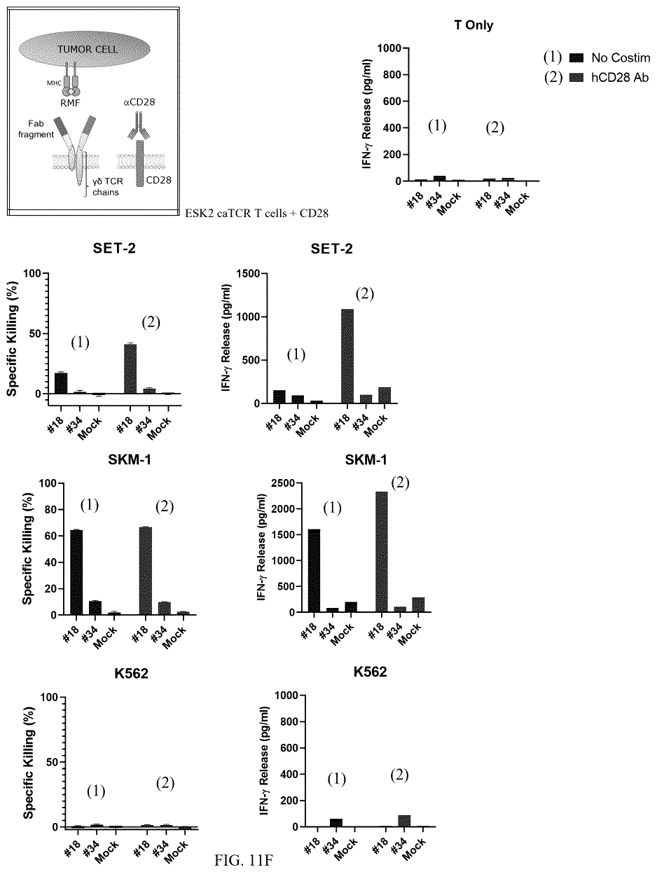

Noticeably, ESK2 caTCR T cells' potency was significantly higher against SKM-1 than against SET-2. Since both the “first signal” (provided by caTCR) and costimulatory signaling are essential for full activation of T cells, the expression of CD28/4-1BB ligands on these two AML cell lines was investigated (FIG. 11D). Flow cytometry detected high CD86 (CD28 ligand) and CD137L (4-1BB ligand) levels on SKM-1, while only minor CD80 (CD28 ligand) expression was detected on SET-2. Without wishing to be bound by theory, it is believed that the abundant co-stimulation signal from SKM-1 contributed to its killing by ESK2 caTCR T cells. To test this hypothesis, anti-human CD28 antibody was added into the co-cultures to activate CD28 signal pathway in caTCR T cells regardless of CD28 ligand levels on tumor cells. A significant boost of killing activity against SET-2 was observed in contrast to against SKM-1 (FIG. 11E), emphasizing the importance of costimulatory signaling for robust caTCR T cell activation. This hypothesis was further validated by the IFN-gamma secretion from ESK2 caTCR T cells with or without anti-CD33 CSR after co-culture with SET-2 (FIG. 12).

### Example 3: Cytotoxicity of T Cells Comprising ESK2 caTCR and Anti-CD33 CSR Against Myeloid Leukemia Requires Both Primary and Costimulatory Signals

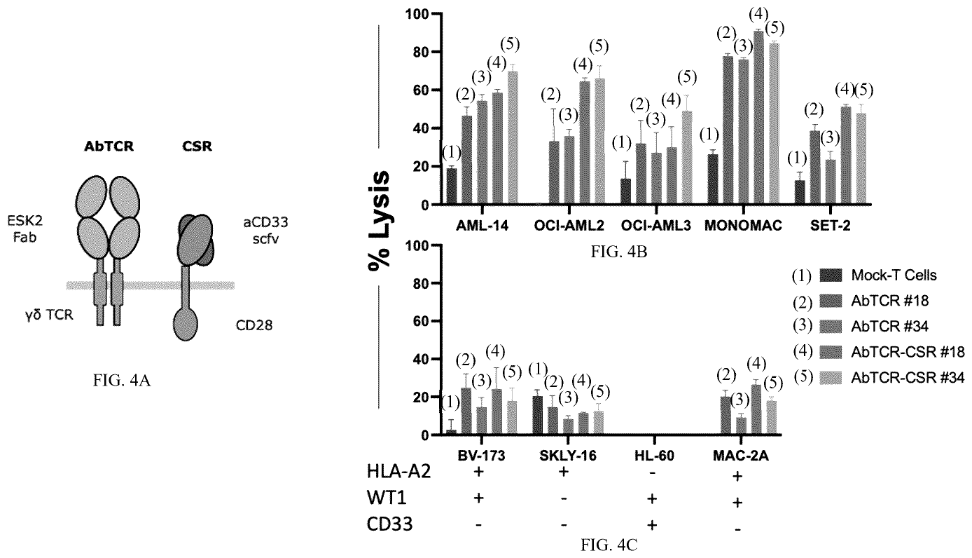

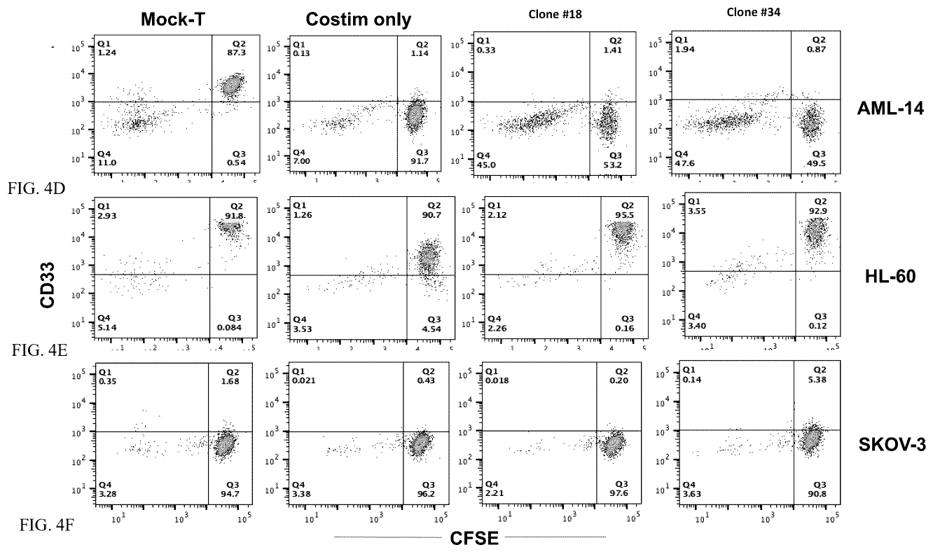

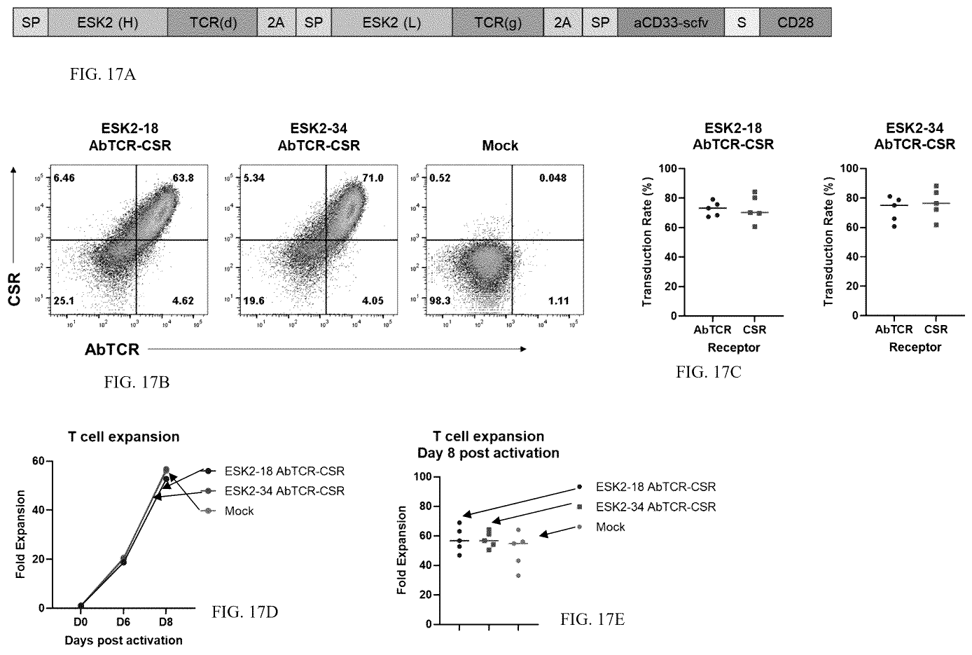

Based on the high cytolytic activity and specificity of ESK2 clones #18 and #34, these antigen recognition domains along with the lintuzumab (22) anti-CD33 scFv were engineered into the caTCR and anti-CD33 CSR constructs. The constructs consisted of an ESK2 caTCR as the primary signaling receptor, which comprised either clone #18 or clone #34 Fab linked to truncated gamma/delta TCR as the effector domain, and a costimulatory receptor anti-CD33 CSR, in which the anti-CD33 scFv is fused to the transmembrane and intracellular signaling domains of CD28. It was hypothesized that the T cells expressing such a receptor combination would be fully activated with both TCR/CD3 and CD28 costimulatory signals, in a way that resembles the “two-signal model” of natural T cell activation, hence increasing the specificity against CD33+/WT1+ tumor cells. Meanwhile, with the two-component activation design, “on-target/off-tumor” and off target toxicities should be reduced. A scheme of the AbTCR-CSR and its constructs is shown in FIG. 4A and vector map in FIG. 17A. Transduction efficiency of the AbTCR-CSR clone #18 and #34 and their cell expansion are shown in FIGS. 17B-17E.

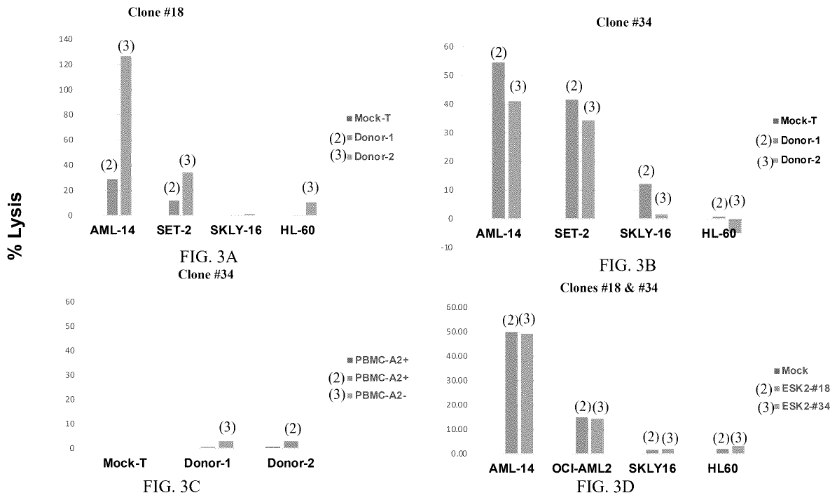

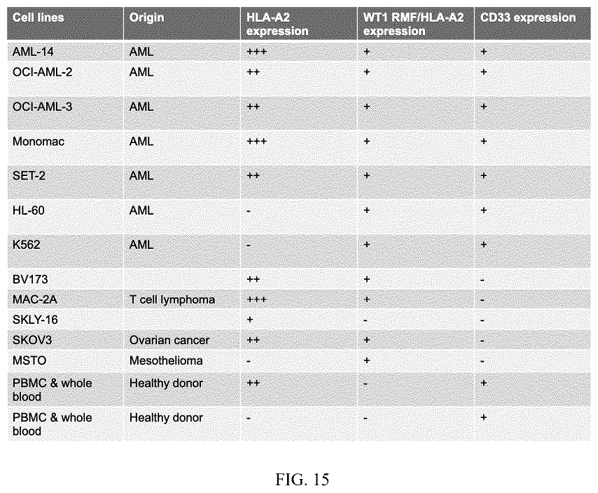

We tested the cytotoxicity of both ESK2-18 and -34 AbTCR and AbTCR-CSR against a panel of leukemia cell lines. AbTCR of both clones were able to kill the AML cell lines that were triple positive for WT1/HLA-A2 and CD33. The cytotoxicity was further enhanced, depending on the target cell line, in AbTCR-CSR T cells (FIGS. 4B-4C). However, no significant enhancement of the cytotoxicity by AbTCR-CSR T cells was observed against the cell lines that were CD33 negative such as BV173, MAC-2A and SKLY-16. No cytotoxicity against HL-60, which is WT1 positive and CD33 positive, but HLA-A2 negative, showing that the primary signal targeting WT1 RMF is a critical factor for cytotoxicity (FIG. 3B). Phenotypes of the cell lines are listed in FIG. 15. These results demonstrated our hypothesis that that ESK2-CD33 AbTCR-CSR T cells were potent and specific cells against target cells simultaneously expressing all three target proteins.

The cytolytic activity of T cells comprising ESK-2 caTCR and anti-CD33 CSR was tested against a panel of AML and CML cell lines that are WT1, CD33 and HLA-A2 positive or negative as listed in FIG. 15. Both ESK2 clones #18 and clone #34 were transduced in two different donors as the primary receptor and each showed cytotoxicity against AML-14 and SET-2 AML cell lines that are WT1+, CD33+ and HLA-A0201+, but not to cell lines SKLY-16 (CD33−) or HL-60 (non-HLA-A2), (FIGS. 3A-3B). However, no killing was seen against PBMCs from HLA-A2 positive or negative healthy donors (FIG. 3C). When T cells comprising anti-CD33 CSR with ESK2 caTCR clone #18 and clone #34 were studied in the same experiment, both killed AML-14 and OCI-AML-2, to a similar degree (FIG. 3D). These results demonstrated that ESK2 caTCR-anti-CD33 CSR expressing T cells were potent against cells simultaneously expressing all 3 target proteins: WT1, CD33 and HLA-A2.

The coordination and necessity of the two stimulatory signals was assessed next. We engineered T cells in which ESK2 Fab was replaced with an irrelevant Fab, leaving the CD33 costimulatory receptor as before: “costim only”. Four target cell lines were tested: AML-14 is WT1, CD33 and HLA-A2 triple positive; HL-60 and K562 are HLA-A2 negative; SKOV3/A2 is a WT1 positive, CD33 negative and HLA-A2+ transduced ovarian cancer cell line (FIG. 15). All target cells were CFSE-labeled and incubated with caTCR plus CSR bispecific T cells overnight and the killing was measured by flow cytometry-based analysis after staining cells with anti-CD33 mAb. Target cells were gated on larger tumor cells based on forward and side scatters, to exclude the effector T cells (smaller in size) and the percentage of CD33 and CFSE double positive target cells was measured (FIGS. 4D-4F). After incubating AML-14 targets with control mock-T cells, there were 88% CD33+CFSE+ cells remaining. T cells with the irrelevant Fab (costim only) showed down-modulation of cell surface CD33 levels on target cells (due to the presence of the CD33 scFv on the effectors that could block the detection mAb or to down modulation of CD33); however, the percentage of live cells was similar to the mock-T control at 93% (right quadrants). This showed that the first signal is absolutely required to activate T cells to kill, and an unengaged irrelevant first signal would not coordinate with the anti-CD33 costimulatory signal.

T cells comprising anti-CD33 CSR with either ESK2 caTCR clone #18 or ESK2 caTCR clone #34 not only showed down-modulation of CD33 levels on target cells, but also killing (50-55%) (FIG. 4D, right quadrants). In HL-60 cells (HLA-A2(−)), no killing was seen in with any effector cells though CD33 surface levels were also reduced in the costim only group (FIG. 4E). In another experiment, K562 cells (HLA-A2 negative) showed a similar result to HL-60. These data support a superior specificity profile of ESK2 caTCR-anti-CD33 CSR expressing T cells. No binding of CD33 nor killing of SKVO3/A2 ovarian cancer cells was observed, as these cells do not express CD33 and percentages of all CFSE+ cells remain similar in all four groups (FIG. 4F).

T cells comprising anti-CD33 CSR with ESK2 caTCR were tested next for their ability to kill primary AML samples and AML PDXs. Four AML primary samples and three AML PDXs were tested as targets and the percentage of CD33+ cells varies among the samples. To assess the elimination of CD33 leukemia cells, the target cells were CFSE-labeled and co-incubated with the T cells comprising anti-CD33 CSR with ESK2 caTCR. The elimination of CD33 high and low populations were calculated in comparison with the mock-T cell groups (Table 4)

TABLE 4
Reduction of primary AML by AbTCR and AbTCR-CSR T cells.

| | | | | | | | WT1 | |
|---|---|---|---|---|---|---|---|---|
| | Reduction of CD33+ cells (%) | | | | | | RMF/HLA- | |
| Primary | CSR | Ab-TCR | Ab-TCR | AbTCR- | AbTCR- | HLA- | A2 complex | CD33 |
| AML | only | #18 only | #34 only | CSR #18 | CSR #34 | A*02:01 | Expression | expression |
| 60D | None | 73 | 28 | 90 | 16 | + | + | 99% |
| 114B | None | 35 | 38 | 81 | 17 | + | + | 54% |
| 92C | None | 57 | 69 | 69 | 74 | + | + | 77% |
| 120B | None | 5.6 | None | None | 2.3 | − | − | 40% |
| AML-14 | None | 41 | 55 | 74 | 55 | + | + | 99% |

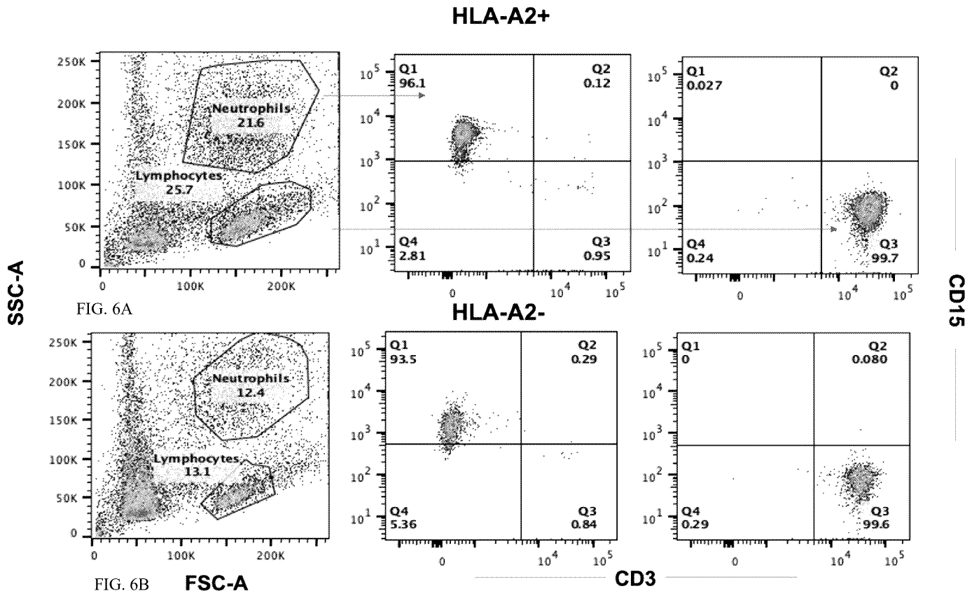

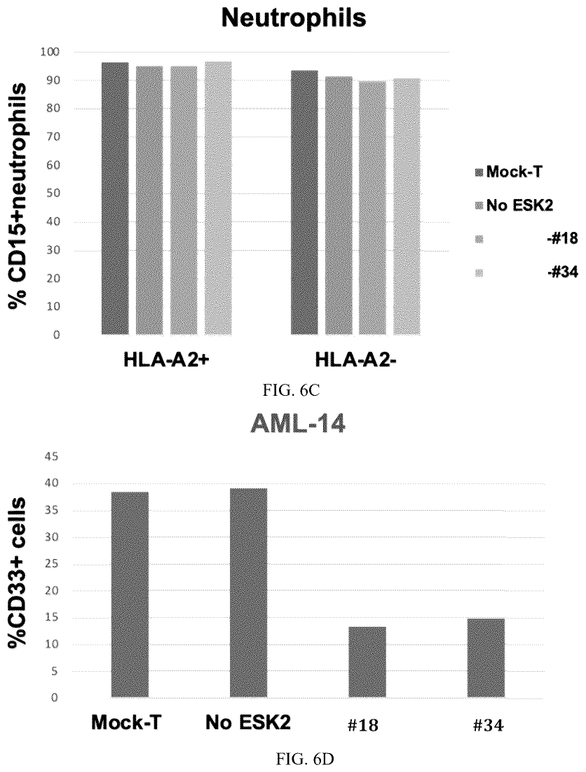

Primary AML samples were labeled with either CFSE or far-red, and co-incubated with ESK2 Ab-TCR or AbTCR-CSR T cells at an E:T ratio of 1:1 overnight. The cells were harvested and were stained with anti-CD33 mAb and analyzed by flow cytometry. The cells were gated on live cells and the same gates were applied to all groups. The percentage of CD33 and CFSE (or far-red) positive cells in the live cell gates were analyzed (as shown in Supp. FIG. 6) and the percentage of total CD33+CFSE/far-red+ were calculated (including CD33^{low} cells in AbTCR-CSR groups). Percentage reduction of the AML cells by AbTCR, AbTCR-CSR T cells was calculated by comparison to mock-T cells. HLA-A2 and CD33 expression were measured by flow cytometric analysis. WT1 expression was assessed by ESK1 and ESK2 staining. “+” = >1.5-fold increase over the isotype control staining for WT1 RMF/HLA-A2, “+” = >20 fold over isotype for HLA-A2 and CD33. AML-14 is a positive control AML cell line. “None” = 0 to 1.5% reduction over Mock-T cell groups.

There was a variable degree of killing among the samples and the bispecific anti-CD33 CSR-ESK-2 caTCR clones, which could be due to the heterogeneity of the primary samples (Table 4). In general, T cells comprising anti-CD33 CSR-ESK-2 caTCR clone #18 showed more potent killing than T cells comprising anti-CD33 CSR-ESK-2 caTCR clone #34, and costim-only T cells did not show the reduction of CD33 high and low cells. No killing was detected against HLA-A*02:01 negative samples 120B and M170. These results demonstrated that the T cells comprising anti-CD33 CSR and ESK-2 caTCR clone #18 or ESK-2 caTCR clone #34 were cytolytic against the primary AML cells that were WT1⁺CD33⁺ and HLA-A2 positive.

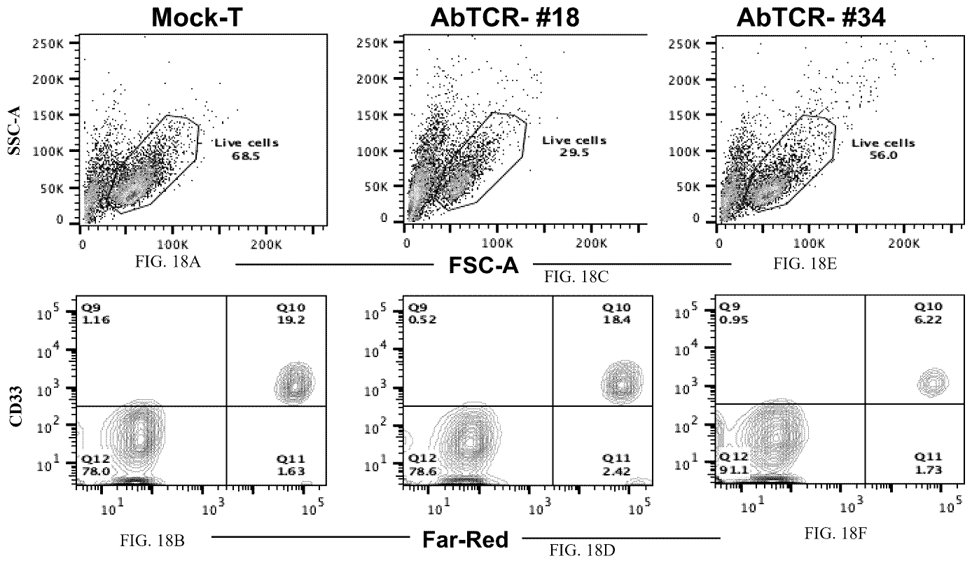

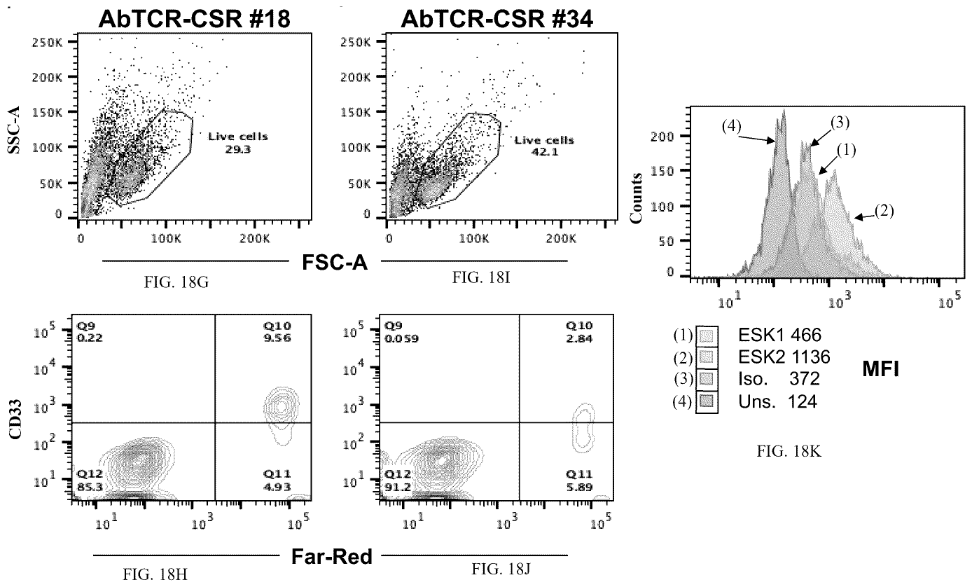

ESK2 AbTCR or AbTCR-CSR T cells were next tested for their ability to kill primary AML samples. Four CD33+ AML primary samples were tested as targets. The elimination of CD33 positive populations were calculated in comparison with the Mock-T cell groups (Table 4.) There was a variable degree of killing among the samples by the effector AbTCR-CSR T cells, which could be due to the heterogeneity of the CD33 on primary samples (Table 4). In general, clone #18 showed more potent killing than clone #34 in both AbTCR and AbTCR-CSR formats, and CSR only T cells did not show the reduction of CD33 positive cells. No significant killing was detected against HLA-A*02:01 negative samples 120B. Representative flow plots from sample 92C were shown in FIG. 18. These results demonstrated that the ESK2 AbTCR and AbTCR-CSR T cells were cytolytic against the primary AML cells that were WT1+/CD33+/HLA-A2+.

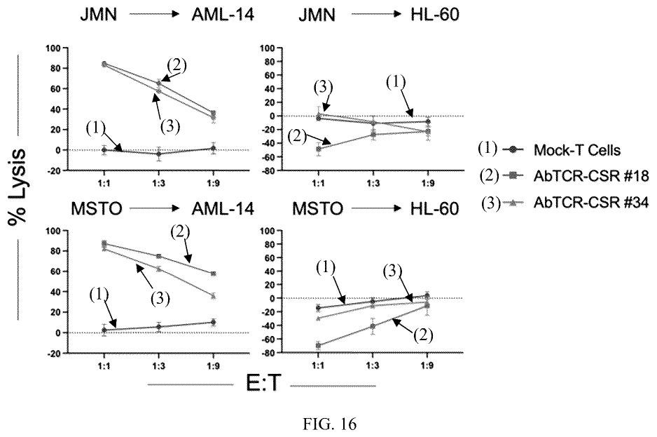

In addition, we asked if such a dual targeting construct could have not only cis activation from both signals on the same cell, but also trans activation from a cell with target #1 and killing of cells that expressed the target #2. AbTCR-CSR #18 and #34 T cells were incubated with JMN (WT1+/HLA-A2+/CD33−) or MSTO (WT1+/HLA-A2−/CD33−) cells overnight and then harvested. The effector cells were then co-incubated with HL-60 cell line (only CD33+) to test if the AbTCR-CSR T cells could acquire activation signal from primary signaling domain, resulting in killing against the target cells expressing only the CSR (CD33). AbTCR-CSR T cells sensitized with JMN were able to kill AML-14 (triple positive), but not HL-60 (Signal #1 negative) (FIG. 16) This suggests that under these transfer conditions and with these lines, cis signaling is required. However, trans signaling may still occur in settings in vivo where multiple target cells are in proximity for long periods of time.

### Example 4: T Cells Comprising Bispecific Anti-CD33 CSR-ESK-2 caTCR Clones were not Cytotoxic to Normal Hematopoietic Cells

Although ESK2 clone #34 was not cytotoxic in IgG or BiTE format against normal hematopoietic cells, a cellular embodiment of ESK2 clone #34 might substantially increase the avidity of the mAb, thereby resulting in killing with a single signal.

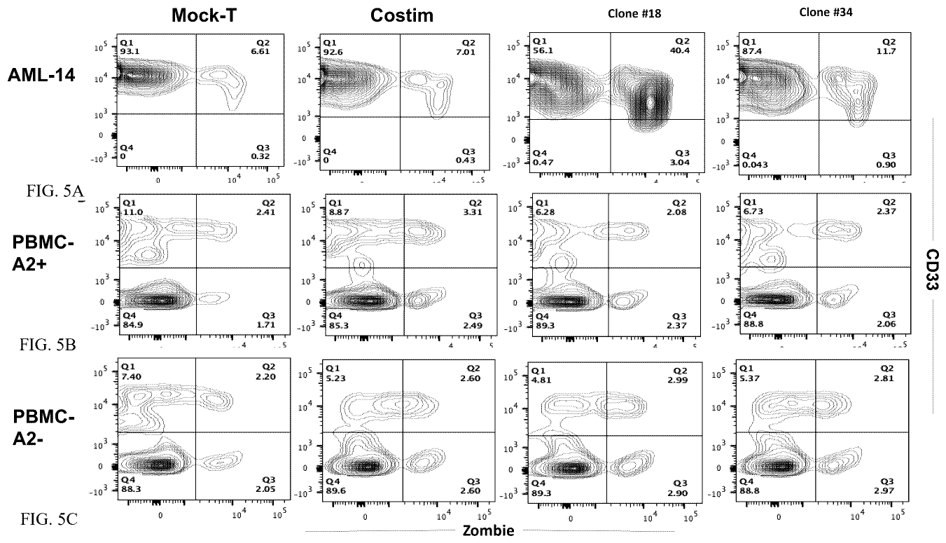

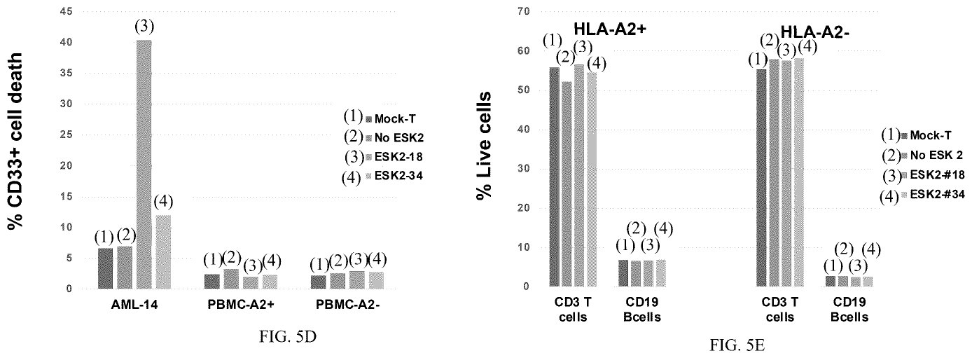

Therefore, we tested if T cells comprising anti-CD33 CSR with ESK2 caTCR clones had cytotoxicity against normal hematopoietic cells. PBMCs from either HLA-A2 positive or negative donors were CFSE-labeled and incubated with effector cells overnight. The cells were then stained with mAbs to T, B, and monocyte markers to determine the depletion of subpopulations. For monocytes, cells were stained with anti-CD33 mAb and zombie dye to determine the percentage death in CD33+CFSE+ target population. While mock-T cells and costim only T cells showed 6.61% and 7% death (CD33+/zombie+), respectively, in control cell line AML-14, T cells comprising anti-CD33 CSR with ESK2 caTCR clones #18 and #34 showed 40% and 11.7% cell death in CD33+ population (FIG. 5A). In contrast, percentage cell death in CD33+ cells were comparable among the 4 groups of T cells from both HLA-A*02:01 positive (FIG. 5B) or negative donors (FIG. 5C). The above data were summarized in FIG. 5D. No significant reduction was observed in CD3+T cell and CD19+B cell compartments in both HLA-A*02:01 positive or negative donor T cells treated with all 4 groups of effectors (FIG. 5E). The results were confirmed in a second set of donors.

Since neutrophils account for the largest cell population in blood and are CD33 positive, we next tested if the T cells comprising anti-CD33 CSR with ESK2 caTCR clones had any cytotoxicity against neutrophils. Neutrophils were isolated from both HLA-A*02:01-positive or negative donors (FIGS. 6A-6B) and were incubated with T cells comprising anti-CD33 CSR with ESK2 caTCR clones at 1:1 E:T ratio overnight. The cells were then stained with CD15 for neutrophils and CD3 for the effector T cells. There was no observed reduction in the percentage of CD15+ neutrophils in either HLA-A*02:01-positive or negative donors (FIG. 6C). However, AML-14 was killed by T cells comprising anti-CD33 CSR and ESK2 caTCR clone #18 or ESK2 caTCR clone #34 in the same experiment as a control (FIG. 6D).

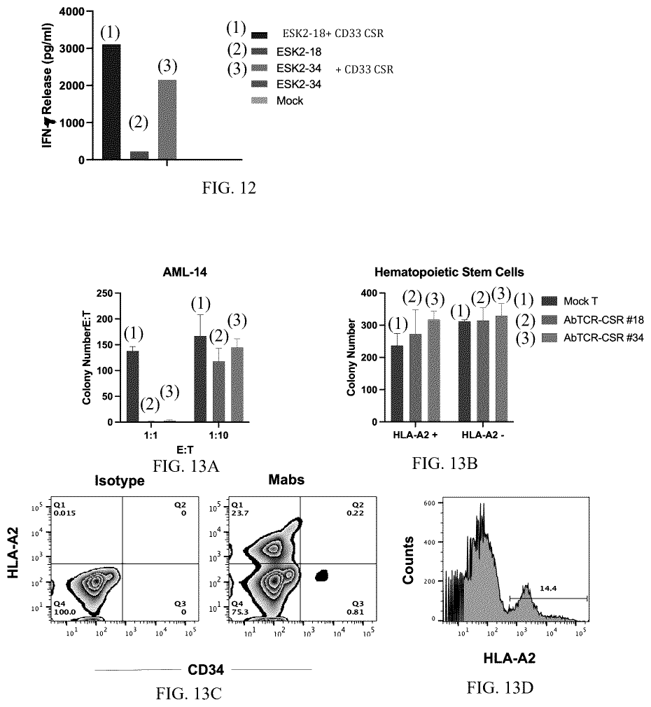

Human hematopoietic stem cells and progenitors have been reported to be CD33 positive (23, 24). Therefore, an important question remained as to whether normal hematopoietic human stem cells (HSCs) would be spared by T cells comprising anti-CD33 CSR with ESK2 caTCR clones. Therefore, we assessed the survival of the HSC and AML-14 cells using colony forming unit (CFU) assays from cord blood isolated HLA-A02 positive and CD34 positive stem cells, using AML-14 leukemia cells as a positive control. There was no reduction in the ability to form colonies among the stem cells (FIG. 13B). In contrast, AML-14 cells formed significantly fewer colonies when treated with the T cells comprising anti-CD33 CSR with ESK2 caTCR clone #18 or clone #34 as compared to a control mock-T cell treatment (FIG. 13A). This sparing of progenitor cells could be explained by the cell surface phenotype of the CD34+ stem cells, which displayed few HLA molecules on their surfaces, thus protecting them from killing (FIG. 13C). Overall, HLA-A2 expression was found on only 14% of cells in the whole cord blood population (FIG. 13D) and similar results were observed in three other HLA-A2+ cord blood samples.

Taken together these data suggest that selective cytotoxicity against cancer targets can be achieved without killing of normal cells expressing CD33, such as monocytes, neutrophils, myeloid progenitors, or other cells that might cross react with just one of the receptors on T cells expressing anti-CD33 CSR with ESK2 caTCRs.

### Example 5: T Cells Comprising Bispecific Anti-CD33 CSR-ESK-2 caTCR Clones Treat AML in NSG Xenograft Mice

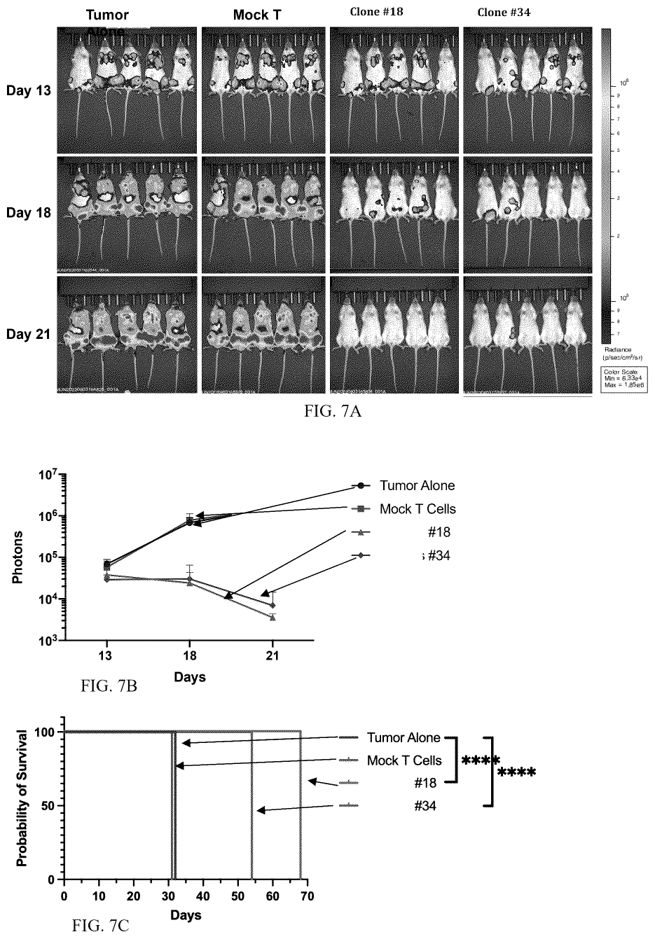

We tested the efficacy of T cells comprising bispecific anti-CD33 CSR-ESK-2 caTCR clones in two models in vivo. In the first cancer model, NSG mice were xenografted intravenously 13 days before treatment with AML-14 cells. At the time of treatment, mice had disseminated leukemia visible in their lung, spleen, and bone marrow (FIG. 7A). T cells comprising anti-CD33 CSR with ESK2 caTCR clones #18 and clone #34 or mock-T cells were given by i.v. injection. Five days after effector cell injection, a reduction of tumor cells was observed, which continued to improve through eight days that became more visible eight days post injection.

T cells comprising anti-CD33 CSR with either ESK2 caTCR clone #18 or ESK2 caTCR clone #34 nearly eliminated leukemia in all mice in comparison to control groups of untreated mice or mice treated with mock-T cells. BLI summation from five mice showed more than a hundred-fold reduction of tumor burden on day 21 in the mice treated with T cells comprising anti-CD33 CSR with either ESK2 caTCR clone #18 or ESK2 caTCR clone #34 compared to control mice (FIG. 7B). 31 days after tumor engraftment, all mice in the mock-T cell injected group and untreated group died due to infiltration of the AML cells; there was significant prolongation in survival in the T cells expressing anti-CD33 CSR with either ESK2 caTCR clone #18 or ESK2 caTCR clone #34 injected groups (FIG. 7C). Mice in each group seem to have developed a leukemia burden at a similar rate; they either died or were sacrificed at a similar time due to hind limb paralysis.

In the second, more aggressive AML model, T cells expressing anti-CD33 CSR with either ESK2 caTCR clone #18 or ESK2 caTCR clone #34, “costim only” T cells, or mock-T cells were given by i.v. injection twice on day 4 and day 12 after OCI-AML-2 cells were xenografted. Substantial tumor inhibition was seen in the groups of mice that received T cells expressing anti-CD33 CSR with either ESK2 caTCR clone.

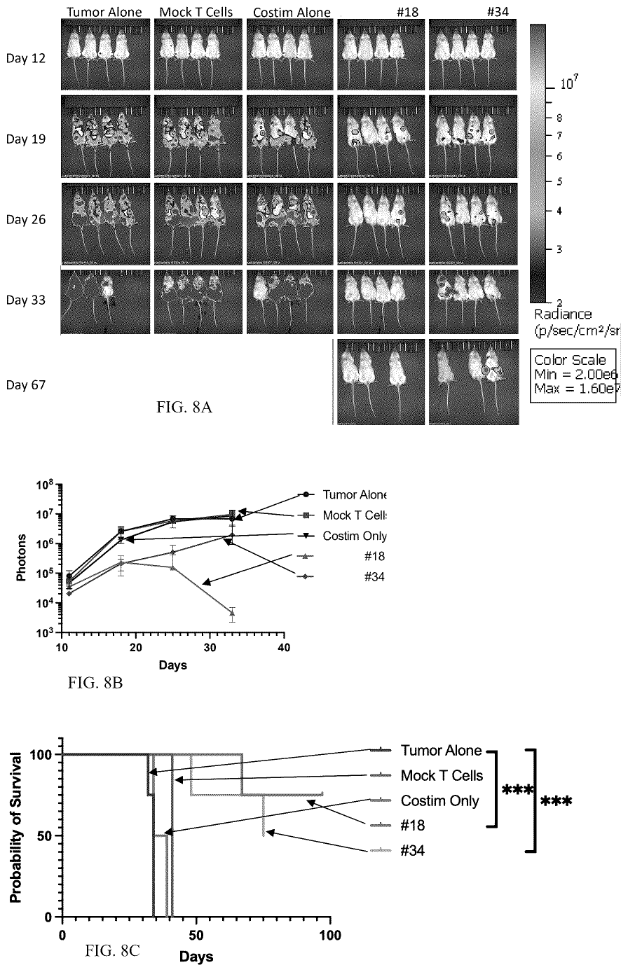

On day 33, mice that received T cells expressing anti-CD33 CSR with ESK2 caTCR clone #18 were tumor-free by BLI and only one mouse from the T cells expressing anti-CD33 CSR with ESK2 caTCR clone #34 treated group showed visible tumor burden (FIG. 8A). This mouse died on day 48. On day 67 post tumor injection, in the bispecific anti-CD33 CSR-ESK-2 caTCR clone #18-treated group, mice remained tumor-free, except for one mouse that died (tumor free) without explanation; the remaining 3 mice were sacrificed on day 75 as a termination of the experiment. Two mice from the bispecific anti-CD33 CSR-ESK-2 caTCR clone #34-treated group also were tumor free. The remaining mouse with a tumor was sacrificed due to tumor growth on day 75 and two tumor-free mice were sacrificed as a result of an error at the mouse facility.

Total BLI flux (photons/second) for each treatment group and survival curves of the mice showed strong inhibition of tumor growth by T cells expressing anti-CD33 CSR with ESK2 caTCR clone #18 or ESK2 caTCR clone #34 (FIGS. 8B and 8C). Importantly, mice treated with T cells expressing an irrelevant primary recognition domain and the CD33 reactive costimulatory domain (the “Costim Only” group) did not show any tumor inhibition, which was consistent with in vitro data, demonstrating the indispensability of the primary signal for T cell-mediated cytotoxicity.

These results demonstrate that the anti-WT1 peptide/MHC TCRm (ESK2), compositions of the present technology are useful for treating cancer.

### Example 6: Comparative Analysis with Variant ESK2 V_{H} and V_{L} Domains of the Present Disclosure

To compare the specificity and affinity of ESK2 variants, exemplary heavy (SEQ ID NOs: 123-125 and 130-133) and light chain (SEQ ID NOs: 126-129 and 134-136) sequences are synthesized and cloned into a BiTE format with an N-terminal signal sequence and myc tag. The BiTE clones are expressed in HEK cells and purified. They are tested in an in vitro assay for binding and specificity using peptide-loaded T2 cells.

Binding of BiTEs containing framework (FW) mutations to peptide/HLA-A2 complexes on live cell surfaces is determined using a TAP-deficient, HLA-A2+ cell line, T2 as described above. The FW mutant BiTE clones are tested for robust and specific binding to T2 cells pulsed with RMF peptide, but not T2 cells left unpulsed or pulsed with the irrelevant control HLA-A2-binding peptides. Individual FW mutant BiTE clones are tested against RMF/HLLA-A*02:01 complex loaded T2 cells (50 μg/million cells) or T2 cells loaded with control peptides (T2 cell loaded with a mixture of 20 control peptides, 50 μg/million cells), respectively. Binding of the FW mutant BiTE clones is detected by staining cells with a PE-conjugated BB7.2 antibody (HLA-A*0 (2 specific; Invitrogen Cat #25-9876-42) and analyzed by FACS for BB7.2 binding.

It is anticipated that clones with mutations in framework regions show comparable binding and selectivity as compared to their corresponding parent clones (ESK2 clone 18 or 34).

SEQUENCE SUMMARY

| SEQ ID | |
|---|---|
| NO | Sequence |
| 1 | GYNFTSYG |
| 2 | ISAYNGNT |
| 3 | ARDWDYDFLTGWDGMDV |
| 4 | SSNIGNNY |
| 5 | YNN |
| 6 | GTWDSSLSAGQV |
| 7 | GYTFTDYY |
| 8 | IDPDNGGT |
| 9 | ARALWFGYGFLDY |
| 10 | SSDLGGYPF |
| 11 | DVT |
| 12 | TSYTDANTLV |
| 13 | GFTFDDYA |
| 14 | ISGDGGST |
| 15 | AKDMVAAAGWNPYYYYYGMDV |
| 16 | SGGIATNY |
| 17 | VDN |
| 18 | QSYDSNNHVV |
| 19 | GYTFTGYY |
| 20 | INPNSGGT |
| 21 | ARHSSRDHYYYGMDV |
| 22 | SSNIGNNY |
| 23 | ENN |
| 24 | GTWDSSLNAGV |
| 25 | GITVSNNY |
| 26 | IYSDGST |
| 27 | ASDKVAVAWTDGLDS |
| 28 | SLRSYY |
| 29 | GQT |
| 30 | SSRDSSGNHWV |
| 31 | GYTFTDYY |
| 32 | VDPYDGYT |
| 33 | ARFSGTRQDS |
| 34 | SSDVGGYNY |
| 35 | EVS |
| 36 | SSYAGSNNLAVV |
| 37 | GYTFTDFY |
| 38 | VNPKSGGT |
| 39 | ARSWQGMSWESVEDV |
| 40 | SSNIGAGYD |
| 41 | GNS |
| 42 | QSYDSSLSGAV |
| 43 | GYSFTSYW |
| 44 | IYPGDSDT |
| 45 | ARGGYIYYDA |
| 46 | RSNVGNNA |
| 47 | YDD |
| 48 | AAWDDSLNGLV |
| 49 | GFTFSSYA |
| 50 | ISGSGGST |
| 51 | RWGWGPVGRVESHTSGDS |
| 52 | SSNIGAGYV |
| 53 | DNS |
| 54 | QSYDSSLSGWV |
| 55 | GFTFDDYA |
| 56 | ISWNSGSI |
| 57 | ARGYMGHNWYD |
| 58 | DIGSKA |
| 59 | YNR |
| 60 | QVWDSFSDHYV |
| 61 | GFTFSSYW |
| 62 | INSDGSST |
| 63 | ARSGYYMSDI |
| 64 | NIGSKS |
| 65 | YDS |
| 66 | QVWDSSSDQYV |
| 67 | GDTFSSYA |
| 68 | INLIFGTV |
| 69 | ARGHWSQVWWTSHSYDL |
| 70 | NIGSKS |
| 71 | DDS |
| 72 | QVWDSSSDHYV |
| 73 | GYIFTGYY |
| 74 | INPNSGVT |
| 75 | AREGVWGYYDS |
| 76 | SLRSYY |
| 77 | DKN |
| 78 | NSRDSSGNHHVV |
| 79 | GYSFTSYW |
| 80 | IDPSDSYT |
| 81 | ARMEIYSPDY |
| 82 | QSINKY |
| 83 | GAL |
| 84 | QQSYSTPLT |
| 85 | EVQLVQSGAEVKKPGASVKVSCKASGYNFTSYGISWVRQAPGQGLEWMGWISAYNGNTNYAQKL |
| | QGRVTMTTDTSTSTAYMELRSLRSDDTAVYYCARDWDYDFLTGWDGMDVWGQGTTVTVSS |
| 86 | QSVVTQPPSVSAAPGQKVTISCSGSSSNIGNNYVSWYQQLPGTAPKLLIYYNNKRPSGIPDRFS |
| | GSKSGTSATLGITGLQTGDEADYYCGTWDSSLSAGQVFGGGTKLTVLG |
| 87 | QVQLVQSGAEVKKPGASVKVSCKASGYTFTDYYIHWVRQAPGQGLEWMGWIDPDNGGTNY |
| | AQNFQDRVTMTRDTSVSTAYLEVTSLKSDDAAVYYCARALWFGYGFLDYHWGQGTLVTVSS |
| 88 | QSALTQPASVSGSPGQSITISCTGSSSDLGGYPFVSWYQQHPDKAPKLIIYDVTNRPSGISNRFS |
| | GSKSGYRASLTISGLQAEDEADYYCTSYTDANTLVFGTGTKVTVLG |
| 89 | EVQLVESGGGVVQPGGSLRLSCAASGFTFDDYAMHWVRQAPGKGLEWVSLISGDGGSTYYADSVK |
| | GRFTISRDNSKNSLYLQMNSLRTEDTALYYCAKDMVAAAGWNPYYYYYGMDVWGQGTTVTVSS |
| 90 | NFMLTQPHSVSESPGKTVTISCTRSSGGIATNYVQWYQQRPGNAPTAVIYVDNERPSGVPERFS |
| | GSIDTSSNSASLTISGLETEDEADYYCQSYDSNNHVVFGGGTKVTVLG |
| 91 | EVQLVQSGAEVKKPGASVKVSCKASGYTFTGYYMHWVRQAPGQGLEWMGWINPNSGGTNYAQKF |
| | QGRVTMTRDTSISTAYMELGRLRSDDTAVYYCARHSSRDHYYYGMDVWGQGTTVTVSS |
| 92 | QSVVTQPPSVSAAPGQRVTISCSGGSSNIGNNYVSWYQHLPGTAPKLLIYENNLKRPSGIPDRF |
| | SGSKSGTSATLGITGLQTGDEADYYCGTWDSSLNAGVFGGGTKLTVLG |
| 93 | EVQLVESGGGLVQPGGSLRLSCVVSGITVSNNYMTWVRQAPGKGLEWVSVIYSDGSTYYADS |
| | VRGRFTISRDISKNTVFLQMNSLRAEDTAMYYCASDKVAVAWTDGLDSWGQGTMVTVSS |
| 94 | SSELTQDPTVSVALGQTVRITCQGDSLRSYYGGWYQQKPGQAPILVFHGQTNRPSGIPDRFSGS |
| | SSGNTVSLTITGAQAEDEADYYCSSRDSSGNHWVFGGGTKLTVLG |
| 95 | EVQLVQSGAEVKKPGASVKISCKTSGYTFTDYYLHWVRQAPVQGLEWMGYVDPYDGYTHY |
| | AQNFQGRVTMTTDTSTSTAYMELSSLRSEDTAVYYCARFSGTRQDSHWGQGTLVTVSS |
| 96 | QSALTQPPSASGSPGQSVTISCTGTSSDVGGYNYVSWYQQHPGKAPKLMIYEVSKRPSGVPDR |
| | FSGSKSGNTASLTVSGLQAEDEADYYCSSYAGSNNLAVVFGGGTKLTVLG |
| 97 | QVQLVQSGAEVKKPGASVKVSCKASGYTFTDFYIHWVRQAPGQGPEWMGWVNPKSGGTNYAQK |
| | FQGRVTMTRDTSISTAYMALSRLRSDDTAVYYCARSWQGMSWESVEDVWGQGTLVTVSS |
| 98 | QSVLTQPPSVSGAPGQRVTISCTGSSSNIGAGYDVHWYQQLPGTAPKLLIYGNSNRPSGVPDRF |
| | SGSKSGTSASLAITGLQAEDEADYYCQSYDSSLSGAVFGTGTKVTVLG |
| 99 | EVQLVQSGAEVKKPGESLKISCKGSGYSFTSYWIGWVRQMPGKGLEWMGIIYPGDSDTRYSP |
| | SFQGQVTISADKSISTAYLQWSSLKASDTAVYYCARGGYIYYDAWGQGTLVTVSS |
| 100 | SYVLTQPPSVSEAPRQRVTISCSGSRSNVGNNAVSWYQHLPGKAPKLLIYYDDLMPSGVSDRF |
| | SGSKSGTSASLAISGLQSEDEADYYCAAWDDSLNGLVFGGGTKLTVLG |
| 101 | EVQLVESGGGLVQPGGSLRLSCAASGFTFSSYAMSWVRQAPGKGLEWVSAISGSGGSTYYADSVK |
| | GRFTISRDNSKNTLYLQMNSLRAEDTAVYYCARWGWGPVGRVESHTSGDSWGQGTLVTVSS |
| 102 | QSVLTQPPSVSGAPGQRVSISCTGSSSNIGAGYVVHWYQHLPGTVPKLLIYDNSNRPSGVPDRF |
| | SGSNSGSSASLAVTGLQAEDEADYYCQSYDSSLSGWVFGGGTKLTVLG |
| 103 | QVQLVQSGGGLVQPGRSLRLSCAASGFTFDDYAMHWVRQAPGKGLEWVSGISWNSGSIGYA |
| | DSVKGRFTISRDNAKNSLYLQMNSLRPEDTAVYYCARGYMGHNWYDWGQGTLVTVSS |
| 104 | LPVLTQPPSVSVAPGKTARITCGGNDIGSKAVHWYQQKPGQAPVLVIYYNRDRPSGIPERFSGS |
| | NSGNTATLTISRVEAGDEADYYCQVWDSFSDHYVFGTGTKVTVLG |
| 105 | QVQLQESGGGLVQPGGSLRLSCAASGFTFSSYWMHWVRQAPGKGLVWVSRINSDGSSTSYA |
| | DSVKGRFTISRDNAKNTLYLQMNSLRAEDTAVYYCARSGYYMSDIWGQGTLVTVSS |
| 106 | NFMLTQPPSLSVAPGKTATITCGGNNIGSKSVHWYQQKPGQAPVLVISYDSDRPSGIPERFSGS |
| | KSGNTATLTISRVEAGDEADYYCQVWDSSSDQYVFGSGTKVTVLG |
| 107 | QVQLVQSGAEVKKPGSSVKVSCKASGDTFSSYAVSWVRQAPGQGLEWMGAINLIFGTVKYAQKFQ |
| | GRITITADESTSTAYMELSSLRSEDTAVYYCARGHWSQVWWTSHSYDLWGQGTLVTVSS |
| 108 | QAVLTQPPSVSVAPGKTARITCGGNNIGSKSVHWYQQKPGQAPVLVVYDDSDRPSGIPERFSG |
| | SNSGNTATLTISRVEAGDEADYYCQVWDSSSDHYVFGTGTKVTVLG |
| 109 | QVQLVQSGTEVRKPGASLKVSCTTSGYIFTGYYIHWVRQVPGQGLEWMGWINPNSGVTEFAQ |
| | GFQGRITMTRDTSTSTVYMELSRLTSDDTAVYYCAREGVWGYYDSWGQGTLVTVSS |
| 110 | SSELTQDPAVSVALGQTVRITCQGDSLRSYYASWYQQKPGQAPVLVIYDKNNRPSGIPDRFSG |
| | SSSGNTASLTITGAQAEDEADYYCNSRDSSGNHHVVFGGGTKLTVLG |
| 111 | EVQLVQSGAEVKKPGESLRISCKDSGYSFTSYWISWVRQMPGKGLEWMGRIDPSDSYTNYSP |
| | SFQGHVTISADKSISTAYLQWSSLKASDTAMYYCARMEIYSPDYWGQGTLVTVSS |
| 112 | DIQLTQSPSSLSASVGDRVTISCRASQSINKYLNWYQQKPGEAPKLLIYGALRLQSGVPSRFSGS |
| | GSGTDYALTITSLQPEDFATYYCQQSYSTPLTFGGGTKVDIKR |
| 113 | RMFPNAPYL |
| 114 | RMAAADPYL |
| 115 | AMFPNAPYL |
| 116 | RMAPNAPYL |
| 117 | RMFANAPY |
| 118 | RMFPAAPYL |
| 119 | RMFPNGPYL |
| 120 | RMFPNAAYL |
| 121 | RMFPNAPAL |
| 122 | YMLDLQPET |
| 123 | EVQLLESGGGLVQPGGSLRLSCAASGFTFSSYAMSWVRQAPGKGLEWVSAISGSGGSTYYGDSVK |
| | GRFTISRDNSKNTLYLQMNSLRAEDTAVYYCARWGWGPVGRVESHTSGDSWGQGTLVTVSS |
| 124 | EVQLVESGGGLVQPGGSLRLSCAASGFTFSSYAMSWVRQAPGKGLEWVSAISGSGGSTYYGDSVK |
| | GRFTISRDNSKNTLYLQMNSLRAEDTAVYYCARWGWGPVGRVESHTSGDSWGQGTLVTVSS |
| 125 | EVQLLESGGGLVQPGGSLRLSCAASGFTFSSYAMSWVRQAPGKGLEWVSAISGSGGSTYYADSVK |
| | GRFTISRDNSKNTLYLQMNSLRAEDTAVYYCARWGWGPVGRVESHTSGDSWGQGTLVTVSS |
| 126 | QSVLTQPPSVSGAPGQRVTISCTGSSSNIGAGYVVHWYQQLPGTAPKLLIYDNSNRPSGVPDR |
| | FSGSKSGTSASLAVTGLQAEDEADYYCQSYDSSLSGWVFGGGTKLTVL |
| 127 | QSVLTQPPSVSGAPGQRVTISCTGSSSNIGAGYVVHWYQHLPGTAPKLLIYDNSNRPSGVPDRF |
| | SGSNSGSSASLAVTGLQAEDEADYYCQSYDSSLSGWVFGGGTKLTVL |
| 128 | QSVLTQPPSVSGAPGQRVSISCTGSSSNIGAGYVVHWYQQLPGTVPKLLIYDNSNRPSGVPDRF |
| | SGSNSGTSASLAVTGLQAEDEADYYCQSYDSSLSGWVFGGGTKLTVL |
| 129 | QSVLTQPPSVSGAPGQRVTISCTGSSSNIGAGYVVHWYQHLPGTVPKLLIYDNSNRPSGVPDRF |
| | SGSKSGTSASLAVTGLQAEDEADYYCQSYDSSLSGWVFGGGTKLTVL |
| 130 | QVQLVQSGAEVKKPGASLKVSCTTSGYIFTGYYIHWVRQAPGQGLEWMGWINPNSGVTEFA |
| | QGFQGRITMTRDTSTSTAYMELSRLTSDDTAVYYCAREGVWGYYDSWGQGTLVTVSS |
| 131 | QVQLVQSGTEVKKPGASLKVSCTTSGYIFTGYYIHWVRQVPGQGLEWMGWINPNSGVTEFA |
| | QGFQGRITMTRDTSTSTAYMELSRLTSDDTAVYYCAREGVWGYYDSWGQGTLVTVSS |
| 132 | QVQLVQSGAEVRKPGASLKVSCTTSGYIFTGYYIHWVRQAPGQGLEWMGWINPNSGVTEFA |
| | QGFQGRITMTRDTSTSTVYMELSRLTSDDTAVYYCAREGVWGYYDSWGQGTLVTVSS |
| 133 | QVQLVQSGTEVKKPGASLKVSCTTSGYIFTGYYIHWVRQAPGQGLEWMGWINPNSGVTEFA |
| | QGFQGRITMTRDTSTSTAYMELSRLTSDDTAVYYCAREGVWGYYDSWGQGTLVTVSS |
| 134 | SYELTQDPAVSVALGQTVRITCQGDSLRSYYASWYQQKPGQAPVRVIYDKNNRPSGIPDRFSG |
| | SSSGNTASLTITGAQAEDEADYYCNSRDSSGNHHVVFGGGTKLTVL |
| 135 | SYELTQDPAVSVALGQTVRITCQGDSLRSYYASWYQQKPGQAPVLVIYDKNNRPSGIPDRFSG |
| | SSSGNTASLTITGAQAEDEADYYCNSRDSSGNHHVVFGGGTKLTVL |
| 136 | SSELTQDPAVSVALGQTVRITCQGDSLRSYYASWYQQKPGQAPVRVIYDKNNRPSGIPDRFSG |
| | SSSGNTASLTITGAQAEDEADYYCNSRDSSGNHHVVFGGGTKLTVL |
| 137 | EVQLVQSGAEVKKPGASVKVSCKAS |
| 138 | ISWVRQAPGQGLEWMGW |
| 139 | NYAQKLQGRVTMTTDTSTSTAYMELRSLRSDDTAVYYC |
| 140 | WGQGTTVTVSS |
| 141 | QSVVTQPPSVSAAPGQKVTISCSGS |
| 142 | VSWYQQLPGTAPKLLIY |
| 143 | KRPSGIPDRFSGSKSGTSATLGITGLQTGDEADYYC |
| 144 | FGGGTKLTVLG |
| 145 | QVQLVQSGAEVKKPGASVKVSCKAS |
| 146 | IHWVRQAPGQGLEWMGW |
| 147 | NYAQNFQDRVTMTRDTSVSTAYLEVTSLKSDDAAVYYC |
| 148 | HWGQGTLVTVSS |
| 149 | QSALTQPASVSGSPGQSITISCTGS |
| 150 | VSWYQQHPDKAPKLIIY |
| 151 | NRPSGISNRFSGSKSGYRASLTISGLQAEDEADYYC |
| 152 | FGTGTKVTVLG |
| 153 | EVQLVESGGGVVQPGGSLRLSCAAS |
| 154 | MHWVRQAPGKGLEWVSL |
| 155 | YYADSVKGRFTISRDNSKNSLYLQMNSLRTEDTALYYC |
| 156 | WGQGTTVTVSS |
| 157 | NFMLTQPHSVSESPGKTVTISCTRS |
| 158 | VQWYQQRPGNAPTAVIY |
| 159 | ERPSGVPERFSGSIDTSSNSASLTISGLETEDEADYYC |
| 160 | FGGGTKVTVLG |
| 161 | EVQLVQSGAEVKKPGASVKVSCKAS |
| 162 | MHWVRQAPGQGLEWMGW |
| 163 | NYAQKFQGRVTMTRDTSISTAYMELGRLRSDDTAVYYC |
| 164 | WGQGTTVTVSS |
| 165 | QSVVTQPPSVSAAPGQRVTISCSGG |
| 166 | VSWYQHLPGTAPKLLIY |
| 167 | LKRPSGIPDRFSGSKSGTSATLGITGLQTGDEADYYC |
| 168 | FGGGTKLTVLG |
| 169 | EVQLVESGGGLVQPGGSLRLSCVVS |
| 170 | MTWVRQAPGKGLEWVSV |
| 171 | YYADSVRGRFTISRDISKNTVFLQMNSLRAEDTAMYYC |
| 172 | WGQGTMVTVSS |
| 173 | SSELTQDPTVSVALGQTVRITCQGD |
| 174 | GGWYQQKPGQAPILVFH |
| 175 | NRPSGIPDRFSGSSSGNTVSLTITGAQAEDEADYYC |
| 176 | FGGGTKLTVLG |
| 177 | EVQLVQSGAEVKKPGASVKISCKTS |
| 178 | LHWVRQAPVQGLEWMGY |
| 179 | HYAQNFQGRVTMTTDTSTSTAYMELSSLRSEDTAVYYC |
| 180 | HWGQGTLVTVSS |
| 181 | QSALTQPPSASGSPGQSVTISCTGT |
| 182 | VSWYQQHPGKAPKLMIY |
| 183 | KRPSGVPDRFSGSKSGNTASLTVSGLQAEDEADYYC |
| 184 | FGGGTKLTVLG |
| 185 | QVQLVQSGAEVKKPGASVKVSCKAS |
| 186 | IHWVRQAPGQGPEWMGW |
| 187 | NYAQKFQGRVTMTRDTSISTAYMALSRLRSDDTAVYYC |
| 188 | WGQGTLVTVSS |
| 189 | QSVLTQPPSVSGAPGQRVTISCTGS |
| 190 | VHWYQQLPGTAPKLLIY |
| 191 | NRPSGVPDRFSGSKSGTSASLAITGLQAEDEADYYC |
| 192 | FGTGTKVTVLG |
| 193 | EVQLVQSGAEVKKPGESLKISCKGS |
| 194 | IGWVRQMPGKGLEWMGI |
| 195 | RYSPSFQGQVTISADKSISTAYLQWSSLKASDTAVYYC |
| 196 | WGQGTLVTVSS |
| 197 | SYVLTQPPSVSEAPRQRVTISCSGS |
| 198 | VSWYQHLPGKAPKLLIY |
| 199 | LMPSGVSDRFSGSKSGTSASLAISGLQSEDEADYYC |
| 200 | FGGGTKLTVLG |
| 201 | EVQLVESGGGLVQPGGSLRLSCAAS |
| 202 | MSWVRQAPGKGLEWVSA |
| 203 | YYADSVKGRFTISRDNSKNTLYLQMNSLRAEDTAVYYCA |
| 204 | WGQGTLVTVSS |
| 205 | QSVLTQPPSVSGAPGQRVSISCTGS |
| 206 | VHWYQHLPGTVPKLLIY |
| 207 | NRPSGVPDRFSGSNSGSSASLAVTGLQAEDEADYYC |
| 208 | FGGGTKLTVLG |
| 209 | QVQLVQSGGGLVQPGRSLRLSCAAS |
| 210 | MHWVRQAPGKGLEWVSG |
| 211 | GYADSVKGRFTISRDNAKNSLYLQMNSLRPEDTAVYYC |
| 212 | WGQGTLVTVSS |
| 213 | LPVLTQPPSVSVAPGKTARITCGGN |
| 214 | VHWYQQKPGQAPVLVIY |
| 215 | DRPSGIPERFSGSNSGNTATLTISRVEAGDEADYYC |
| 216 | FGTGTKVTVLG |
| 217 | QVQLQESGGGLVQPGGSLRLSCAAS |
| 218 | MHWVRQAPGKGLVWVSR |
| 219 | SYADSVKGRFTISRDNAKNTLYLQMNSLRAEDTAVYYC |
| 220 | WGQGTLVTVSS |
| 221 | NFMLTQPPSLSVAPGKTATITCGGN |
| 222 | VHWYQQKPGQAPVLVIS |
| 223 | DRPSGIPERFSGSKSGNTATLTISRVEAGDEADYYC |
| 224 | FGSGTKVTVLG |
| 225 | QVQLVQSGAEVKKPGSSVKVSCKAS |
| 226 | VSWVRQAPGQGLEWMGA |
| 227 | KYAQKFQGRITITADESTSTAYMELSSLRSEDTAVYYC |
| 228 | WGQGTLVTVSS |
| 229 | QAVLTQPPSVSVAPGKTARITCGGN |
| 230 | VHWYQQKPGQAPVLVVY |
| 231 | DRPSGIPERFSGSNSGNTATLTISRVEAGDEADYYC |
| 232 | FGTGTKVTVLG |
| 233 | QVQLVQSGTEVRKPGASLKVSCTTS |
| 234 | IHWVRQVPGQGLEWMGW |
| 235 | EFAQGFQGRITMTRDTSTSTVYMELSRLTSDDTAVYYC |
| 236 | WGQGTLVTVSS |
| 237 | SSELTQDPAVSVALGQTVRITCQGD |
| 238 | ASWYQQKPGQAPVLVIY |
| 239 | NRPSGIPDRFSGSSSGNTASLTITGAQAEDEADYYC |
| 240 | FGGGTKLTVLG |
| 241 | EVQLVQSGAEVKKPGESLRISCKDS |
| 242 | ISWVRQMPGKGLEWMGR |
| 243 | NYSPSFQGHVTISADKSISTAYLQWSSLKASDTAMYYC |
| 244 | WGQGTLVTVSS |
| 245 | DIQLTQSPSSLSASVGDRVTISCRAS |
| 246 | LNWYQQKPGEAPKLLIY |
| 247 | RLQSGVPSRFSGSGSGTDYALTITSLQPEDFATYYC |
| 248 | FGGGTKVDIKR |
| 249 | SRGGGGSGGGGSGGGGSLEMA |
| 250 | GGGGS |
| 251 | SGGG |
| 252 | GGSGGSGGSGGS |
| 253 | RAKRS |
| 254 | GGGGSGGGGS |
| 255 | GSGS |
| 256 | GGSG |
| 257 | GSGAPVKQTLNFDLLKLAGDVESNPGP |
| 258 | GGGGSGGGGSGGGGS |
| 259 | GSGSGS |
| 260 | GGSGGGSG |
| 261 | RAKRSGSGAPVKQTLNFDLLKLAGDVESNPGP |
| 262 | AAATG |
| 263 | GSGSGSGS |
| 264 | GGSGGGSGGGSG |
| 265 | GSGATNFSLLKQAGDVEENPGP |
| 266 | TPLGDTTHTSG |
| 267 | GSGSGSGSGS |
| 268 | RAKRSGSGATNFSLLKQAGDVEENPGP |
| 269 | AAA |
| 270 | GGSGGS |
| 271 | GSRGGGGSGGGGSGGGGSLEMA |
| 272 | GGSG |
| 273 | GGSGGSGGS |
| 274 | EQKLISEEDL |
| 275 | DYKDHDGDYKDHDIDYKDDDDK |
| 276 | DYKDDDDK |
| 277 | HHHHHH |
| 278 | YPYDVPDYA |
| 279 | YPYDVPDYAS |
| 280 | METDTLLLWVLLLWVPGSTG |
| 281 | KRGRKKLLYIFKQPFMRPVQTTQEEDGCSCRFPEEEEGGCEL |
| 282 | QRRKYRSNKGESPVEPAEPCRYSCPREEEGSTIPIQEDYRKPEPACSP |
| 283 | RSKRSRLLHSDYMNMTPRRPGPTRKHYQPYAPPRDFAAYRS |
| 284 | HRRACRKRIRQKLHLCYPVQTSQPKLELVDSRPRRSSTQLRSGASVTEPVAEERGLMSQPLME |
| | TCHSVGAAYLESLPLQDASPAGGPSSPRDLPEPRVSTEHTNNKIEKIYIMKADTVIVGTVKAEL |
| | PEGRGLAGPAEPELEEELEADHTPHYPEQETEPPLGSCSDVMLSVEEEGKEDPLPTAASGK |
| 285 | ALYLLRRDQRLPPDAHKPPGGGSFRTPIQEEQADAHSTLAKI |
| 286 | CWLTKKKYSSSVHDPNGEYMFMRAVNTAKKSRLTDVTL |
| 287 | CARPRRSPAQEDGKVYINMPGRG |
| 288 | KKVAKKPTNKAPHPKQEPQEINFPDDLPGSNTAAPVQETLHGCQPVTQEDGKESRISVQERQ |
| 289 | RVKFSRSADAPAYQQGQNQLYNELNLGRREEYDVLDKRRGRDPEMGGKPRRKNPQEGLYNE |
| | LQKDKMAEAYSEIGMKGERRRGKGHDGLYQGLSTATKDTYDALHMQALPPR |
| 290 | IEVMYPPPYLDNEKSNGTIIHVKGKHLCPSPLFPGPSKPFWVLVVVGGVLACYSLLVTVAFIIF |
| | WVRSKRSRLLHSDYMNMTPRRPGPTRKHYQPYAPPRDFAAYRS |
| 291 | EQKLISEEDLAAAIEVMYPPPYLDNEKSNGTIIHVKGKHLCPSPLFPGPSKPFWVLVVVGGVLA |
| | CYSLLVTVAFIIFWVRSKRSRLLHSDYMNMTPRRPGPTRKHYQPYAPPRDFAAYRS |
| 292 | EQKLISEEDLAAATGPADLSPGASSVTPPAPAREPGHSPQIISFFLALTSTALLFLLFFLTLRFSVV |
| | KRGRKKLLYIFKQPFMRPVQTTQEEDGCSCRFPEEEEGGCEL |
| 293 | AAATGPADLSPGASSVTPPAPAREPGHSPQIISFFLALTSTALLFLLFFLTLRFSVVKRGRKKLL |
| | YIFKQPFMRPVQTTQEEDGCSCRFPEEEEGGCEL |
| 294 | EQKLISEEDLAAATGPTHLPYVSEMLEARTAGHMQTLADFRQLPARTLSTHWPPQRSLCSSDF |
| | IRILVIFSGMFLVFTLAGALFLHQRRKYRSNKGESPVEPAEPCRYSCPREEEGSTIPIQEDYRKPE |
| | PACSP |
| 295 | AAATGPTHLPYVSEMLEARTAGHMQTLADFRQLPARTLSTHWPPQRSLCSSDFIRILVIFSGMF |
| | LVFTLAGALFLHQRRKYRSNKGESPVEPAEPCRYSCPREEEGSTIPIQEDYRKPEPACSP |
| 296 | EQKLISEEDLAAATGAPPLGTQPDCNPTPENGEAPASTSPTQSLLVDSQASKTLPIPTSAPVALS |
| | STGKPVLDAGPVLFWVILVLVVVVGSSAFLLCHRRACRKRIRQKLHLCYPVQTSQPKLELVDS |
| | RPRRSSTQLRSGASVTEPVAEERGLMSQPLMETCHSVGAAYLESLPLQDASPAGGPSSPRDLPE |
| | PRVSTEHTNNKIEKIYIMKADTVIVGTVKAELPEGRGLAGPAEPELEEELEADHTPHYPEQETE |
| | PPLGSCSDVMLSVEEEGKEDPLPTAASGK |
| 297 | AAATGAPPLGTQPDCNPTPENGEAPASTSPTQSLLVDSQASKTLPIPTSAPVALSSTGKPVLDA |
| | GPVLFWVILVLVVVVGSSAFLLCHRRACRKRIRQKLHLCYPVQTSQPKLELVDSRPRRSSTQL |
| | RSGASVTEPVAEERGLMSQPLMETCHSVGAAYLESLPLQDASPAGGPSSPRDLPEPRVSTEHT |
| | NNKIEKIYIMKADTVIVGTVKAELPEGRGLAGPAEPELEEELEADHTPHYPEQETEPPLGSCSD |
| | VMLSVEEEGKEDPLPTAASGK |
| 298 | EQKLISEEDLAAATGDRDPPATQPQETQGPPARPITVQPTEAWPRTSQGPSTRPVEVPGGRAVA |
| | AILGLGLVLGLLGPLAILLALYLLRRDQRLPPDAHKPPGGGSFRTPIQEEQADAHSTLAKI |
| 299 | AAATGDRDPPATQPQETQGPPARPITVQPTEAWPRTSQGPSTRPVEVPGGRAVAAILGLGLVL |
| | GLLGPLAILLALYLLRRDQRLPPDAHKPPGGGSFRTPIQEEQADAHSTLAKI |
| 300 | EQKLISEEDLAAATGTTTPAPRPPTPAPTIASQPLSLRPEACRPAAGGAVHTRGLDFACDIYIWAP |
| | LAGTGCVLLLSLVITLYCQRRKYRSNKGESPVEPAEPCRYSCPREEEGSTIPIQEDYRKPEPACSP |
| 301 | AAATGTTTPAPRPPTPAPTIASQPLSLRPEACRPAAGGAVHTRGLDFACDIYIWAPLAGTGCVL |
| | LLSLVITLYCQRRKYRSNKGESPVEPAEPCRYSCPREEEGSTIPIQEDYRKPEPACSP |
| 302 | EQKLISEEDLAAATGTTTPAPRPPTPAPTIASQPLSLRPEACRPAAGGAVHTRGLDFACDIYIWA |
| | PLAGTGCVLLLSLVITLYCHRRACRKRIRQKLHLCYPVQTSQPKLELVDSRPRRSSTQLRSGAS |
| | VTEPVAEERGLMSQPLMETCHSVGAAYLESLPLQDASPAGGPSSPRDLPEPRVSTEHTNNKIE |
| | KIYIMKADTVIVGTVKAELPEGRGLAGPAEPELEEELEADHTPHYPEQETEPPLGSCSDVMLSV |
| | EEEGKEDPLPTAASGK |
| 303 | AAATGTTTPAPRPPTPAPTIASQPLSLRPEACRPAAGGAVHTRGLDFACDIYIWAPLAGTGCVL |
| | LLSLVITLYCHRRACRKRIRQKLHLCYPVQTSQPKLELVDSRPRRSSTQLRSGASVTEPVAEER |
| | GLMSQPLMETCHSVGAAYLESLPLQDASPAGGPSSPRDLPEPRVSTEHTNNKIEKIYIMKADTV |
| | IVGTVKAELPEGRGLAGPAEPELEEELEADHTPHYPEQETEPPLGSCSDVMLSVEEEGKEDPLP |
| | TAASGK |
| 304 | EQKLISEEDLAAATGTTTPAPRPPTPAPTIASQPLSLRPEACRPAAGGAVHTRGLDFACDIYIWA |
| | PLAGTGCVLLLSLVITLYCALYLLRRDQRLPPDAHKPPGGGSFRTPIQEEQADAHSTLAKI |
| 305 | AAATGTTTPAPRPPTPAPTIASQPLSLRPEACRPAAGGAVHTRGLDFACDIYIWAPLAGTGCVL |
| | LLSLVITLYCALYLLRRDQRLPPDAHKPPGGGSFRTPIQEEQADAHSTLAKI |
| 306 | EQKLISEEDLAAATGTTTPAPRPPTPAPTIASQPLSLRPEACRPAAGGAVHTRGLDFACDIYIWA |
| | PLAGTGCVLLLSLVITLYCKRGRKKLLYIFKQPFMRPVQTTQEEDGCSCRFPEEEEGGCEL |
| 307 | AAATGTTTPAPRPPTPAPTIASQPLSLRPEACRPAAGGAVHTRGLDFACDIYIWAPLAGTGCVL |
| | LLSLVITLYCKRGRKKLLYIFKQPFMRPVQTTQEEDGCSCRFPEEEEGGCEL |
| 308 | IYIWAPLAGTCGVLLLSLVIT |
| 309 | IISFFLALTSTALLFLLFFLTLRFSVV |
| 310 | ILVIFSGMFLVFTLAGALFLH |
| 311 | FWVLVVVGGVLACYSLLVTVAFIIFWV |
| 312 | PVLDAGPVLFWVILVLVVVVGSSAFLLC |
| 313 | VAAILGLGLVLGLLGPLAILL |
| 314 | FWLPIGCAAFVVCILGCILI |
| 315 | LLAGLVAADAVASLLIVGAVFL |
| 316 | ALVVIPIIFGILFAILLVLVFI |
| 317 | EVQLQQSGPELVKPGASVKISCKASGYTFTDYNMHWVKQSHGKSLEWIGYIYPYNGGTGYN |
| | QKFKSKATLTVDNSSSTAYMDVRSLTSEDSAVYYCARGRPAMDYWGQGTSVTVS |
| 318 | DIVLTQSPASLAVSLGQRATISCRASESVDNYGISFMNWFQQKPGQPPKLLIYAASNQGSGVPA |
| | RFSGSGSGTDFSLNIHPMEEDDTAMYFCQQSKEVPWTFGGGTKLEIK |
| 319 | TDYNMH |
| 320 | YIYPYNGGTGYNQKFKS |
| 321 | RGRPAMDY |
| 322 | RASESVDNYGISFMN |
| 323 | AASNQGS |
| 324 | QQSKEVPWT |
| 325 | IPEDTFFPSPESSCDVKLVEKSFETDTNLNFQNLSVIGFRILLLKVAGFNLLMTLRLWSS |
| 326 | GRADCGFTSVSYQQGVLSATILYEILLGKATLYAVLVSALVLMAMVKRKDF |
| 327 | EVKTDSTDHVKPKETENTKQPSKSCHKPKAIVHTEKVNMMSLTVLGLRMLFAKTVAVNFLLT |
| | AKLFFL |
| 328 | PIKTDVITMDPKDNCSKDANDTLLLQLTNTSAYYMYLLLLLKSVVYFAIITCCLLRRTAFCCN |
| | GEKS |
| 329 | GSGEGRGSLLTCGDVEENPGP |
| 330 | SLYPSAPFL |
| 331 | SLYPSAPFLA |
| 332 | EMFPQVPYHL |
| 333 | RLFPNAKFLL |
| 334 | X(LMV)FPNAPY(LVI) |
| 335 | QSVVTQPPSVSAAPGQKVTISCSGSSSNIGNNYVSWYQQLPGTAPKLLIYYNNKRPSGIPDRFS |
| | GSKSGTSATLGITGLQTGDEADYYCGTWDSSLSAGQVFGGGTKLTVLGSRGGGGSGGGGSGG |
| | GGSLEMAEVQLVQSGAEVKKPGASVKVSCKASGYNFTSYGISWVRQAPGQGLEWMGWISA |
| | YNGNTNYAQKLQGRVTMTTDTSTSTAYMELRSLRSDDTAVYYCARDWDYDFLTGWDGMD |
| | VWGQGTTVTVSS |
| 336 | QSALTQPASVSGSPGQSITISCTGSSSDLGGYPFVSWYQQHPDKAPKLIIYDVTNRPSGISNRFS |
| | GSKSGYRASLTISGLQAEDEADYYCTSYTDANTLVFGTGTKVTVLGSRGGGGSGGGGSGGGG |
| | SLEMAQVQLVQSGAEVKKPGASVKVSCKASGYTFTDYYIHWVRQAPGQGLEWMGWIDPDN |
| | GGTNYAQNFQDRVTMTRDTSVSTAYLEVTSLKSDDAAVYYCARALWFGYGFLDYWGQGTL |
| | VTVSS |
| 337 | NFMLTQPHSVSESPGKTVTISCTRSSGGIATNYVQWYQQRPGNAPTAVIYVDNERPSGVPERFS |
| | GSIDTSSNSASLTISGLETEDEADYYCQSYDSNNHVVFGGGTKVTVLGSRGGGGSGGGGSGGG |
| | GSLEMAEVQLVESGGGVVQPGGSLRLSCAASGFTFDDYAMHWVRQAPGKGLEWVSLISGDG |
| | GSTYYADSVKGRFTISRDNSKNSLYLQMNSLRTEDTALYYCAKDMVAAAGWNPYYYYYGM |
| | DVWGQGTTVTVSS |
| 338 | QSVVTQPPSVSAAPGQRVTISCSGGSSNIGNNYVSWYQHLPGTAPKLLIYENNKRPSGIPDRFS |
| | GSKSGTSATLGITGLQTGDEADYYCGTWDSSLNAGVFGGGTKLTVLGSRGGGGSGGGGSGG |
| | GGSLEMAEVQLVQSGAEVKKPGASVKVSCKASGYTFTGYYMHWVRQAPGQGLEWMGWIN |
| | PNSGGTNYAQKFQGRVTMTRDTSISTAYMELGRLRSDDTAVYYCARHSSRDHYYYGMDVW |
| | GQGTTVTVSS |
| 339 | SSELTQDPTVSVALGQTVRITCQGDSLRSYYGGWYQQKPGQAPILVFHGQTNRPSGIPDRFSGS |
| | SSGNTVSLTITGAQAEDEADYYCSSRDSSGNHWVFGGGTKLTVLGSRGGGGSGGGGSGGGGS |
| | LEMAEVQLVESGGGLVQPGGSLRLSCVVSGITVSNNYMTWVRQAPGKGLEWVSVIYSDGST |
| | YYADSVRGRFTISRDISKNTVFLQMNSLRAEDTAMYYCASDKVAVAWTDGLDSWGQGTMV |
| | TVSS |
| 340 | QSALTQPPSASGSPGQSVTISCTGTSSDVGGYNYVSWYQQHPGKAPKLMIYEVSKRPSGVPDR |
| | FSGSKSGNTASLTVSGLQAEDEADYYCSSYAGSNNLAVVFGGGTKLTVLGSRGGGGSGGGGS |
| | GGGGSLEMAEVQLVQSGAEVKKPGASVKISCKTSGYTFTDYYLHWVRQAPVQGLEWMGYV |
| | DPYDGYTHYAQNFQGRVTMTTDTSTSTAYMELSSLRSEDTAVYYCARFSGTRQDSWGQGTL |
| | VTVSS |
| 341 | QSVLTQPPSVSGAPGQRVTISCTGSSSNIGAGYDVHWYQQLPGTAPKLLIYGNSNRPSGVPDRF |
| | SGSKSGTSASLAITGLQAEDEADYYCQSYDSSLSGAVFGTGTKVTVLGSRGGGGSGGGGSGG |
| | GGSLEMAQVQLVQSGAEVKKPGASVKVSCKASGYTFTDFYIHWVRQAPGQGPEWMGWVNP |
| | KSGGTNYAQKFQGRVTMTRDTSISTAYMALSRLRSDDTAVYYCARSWQGMSWESVEDVWG |
| | QGTLVTVSS |
| 342 | SYVLTQPPSVSEAPRQRVTISCSGSRSNVGNNAVSWYQHLPGKAPKLLIYYDDLMPSGVSDRF |
| | SGSKSGTSASLAISGLQSEDEADYYCAAWDDSLNGLVFGGGTKLTVLGSRGGGGSGGGGSGG |
| | GGSLEMAEVQLVQSGAEVKKPGESLKISCKGSGYSFTSYWIGWVRQMPGKGLEWMGIIYPGD |
| | SDTRYSPSFQGQVTISADKSISTAYLQWSSLKASDTAVYYCARGGYIYYDAWGQGTLVTVSS |
| 343 | QSVLTQPPSVSGAPGQRVSISCTGSSSNIGAGYVVHWYQHLPGTVPKLLIYDNSNRPSGVPDRF |
| | SGSNSGSSASLAVTGLQAEDEADYYCQSYDSSLSGWVFGGGTKLTVLGSRGGGGSGGGGSG |
| | GGGSLEMAEVQLVESGGGLVQPGGSLRLSCAASGFTFSSYAMSWVRQAPGKGLEWVSAISGS |
| | GGSTYYADSVKGRFTISRDNSKNTLYLQMNSLRAEDTAVYYCARWGWGPVGRVESHTSGDS |
| | WGQGTLVTVSS |
| 344 | LPVLTQPPSVSVAPGKTARITCGGNDIGSKAVHWYQQKPGQAPVLVIYYNRDRPSGIPERFSGS |
| | NSGNTATLTISRVEAGDEADYYCQVWDSFSDHYVFGTGTKVTVLGSRGGGGSGGGGSGGGG |
| | SLEMAQVQLVQSGGGLVQPGRSLRLSCAASGFTFDDYAMHWVRQAPGKGLEWVSGISWNS |
| | GSIGYADSVKGRFTISRDNAKNSLYLQMNSLRPEDTAVYYCARGYMGHNWYDWGQGTLVT |
| | VSS |
| 345 | NFMLTQPPSLSVAPGKTATITCGGNNIGSKSVHWYQQKPGQAPVLVISYDSDRPSGIPERFSGS |
| | KSGNTATLTISRVEAGDEADYYCQVWDSSSDQYVFGSGTKVTVLGSRGGGGSGGGGSGGGG |
| | SLEMAQVQLQESGGGLVQPGGSLRLSCAASGFTFSSYWMHWVRQAPGKGLVWVSRINSDGS |
| | STSYADSVKGRFTISRDNAKNTLYLQMNSLRAEDTAVYYCARSGYYMSDIWGQGTLVTVSS |
| 346 | QAVLTQPPSVSVAPGKTARITCGGNNIGSKSVHWYQQKPGQAPVLVVYDDSDRPSGIPERFSG |
| | SNSGNTATLTISRVEAGDEADYYCQVWDSSSDHYVFGTGTKVTVLGSRGGGGSGGGGSGGG |
| | GSLEMAQVQLVQSGAEVKKPGSSVKVSCKASGDTFSSYAVSWVRQAPGQGLEWMGAINLIF |
| | GTVKYAQKFQGRITITADESTSTAYMELSSLRSEDTAVYYCARGHWSQVWWTSHSYDLWGQ |
| | GTLVTVSS |
| 347 | SSELTQDPAVSVALGQTVRITCQGDSLRSYYASWYQQKPGQAPVLVIYDKNNRPSGIPDRFSG |
| | SSSGNTASLTITGAQAEDEADYYCNSRDSSGNHHVVFGGGTKLTVLGSRGGGGSGGGGSGGG |
| | GSLEMAQVQLVQSGTEVRKPGASLKVSCTTSGYIFTGYYIHWVRQVPGQGLEWMGWINPNS |
| | GVTEFAQGFQGRITMTRDTSTSTVYMELSRLTSDDTAVYYCAREGVWGYYDSWGQGTLVTV |
| | SS |
| 348 | DIQLTQSPSSLSASVGDRVTISCRASQSINKYLNWYQQKPGEAPKLLIYGALRLQSGVPSRFSGS |
| | GSGTDYALTITSLQPEDFATYYCQQSYSTPLTFGGGTKVDIKRSRGGGGSGGGGSGGGGSLEM |
| | AEVQLVQSGAEVKKPGESLRISCKDSGYSFTSYWISWVRQMPGKGLEWMGRIDPSDSYTNYS |
| | PSFQGHVTISADKSISTAYLQWSSLKASDTAMYYCARMEIYSPDYWGQGTLVTVSS |

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

### REFERENCES

1. R. J. Brentjens, M. L. Davila, I. Riviere, J. Park, X. Wang, L. G. Cowell, S. Bartido, J. Stefanski, C. Taylor, M. Olszewska, O. Borquez-Ojeda, J. Qu, T. Wasielewska, Q. He, Y. Bernal, I. V. Rijo, C. Hedvat, R. Kobos, K. Curran, P. Steinherz, J. Jurcic, T. Rosenblat, P. Maslak, M. Frattini, M. Sadelain, CD19-targeted T cells rapidly induce molecular remissions in adults with chemotherapy-refractory acute lymphoblastic leukemia. Sci Transl Med 5, 177ra138 (2013).
2. A. G. Chapuis, G. B. Ragnarsson, H. N. Nguyen, C. N. Chaney, J. S. Pufnock, T. M. Schmitt, N. Duerkopp, I. M. Roberts, G. L. Pogosov, W. Y. Ho, S. Ochsenreither, M. Wolfl, M. Bar, J. P. Radich, C. Yee, P. D. Greenberg, Transferred WT1-reactive CD8+T cells can mediate antileukemic activity and persist in post-transplant patients. Sci Transl Med 5, 174ra127 (2013).
3. J. N. Brudno, J. N. Kochenderfer, Recent advances in CAR T-cell toxicity: Mechanisms, manifestations and management. Blood Rev 34, 45-55 (2019).
4. S. Rafiq, C. S. Hackett, R. J. Brentjens, Engineering strategies to overcome the current roadblocks in CAR T cell therapy. Nat Rev Clin Oncol 17, 147-167 (2020).
5. M. Sadelain, R. Brentjens, I. Riviere, The basic principles of chimeric antigen receptor design. Cancer Discov 3, 388-398 (2013).
6. C. H. June, M. Sadelain, Chimeric Antigen Receptor Therapy. N Engl J Med 379, 64-73 (2018).
7. Y. Xu, Z. Yang, L. H. Horan, P. Zhang, L. Liu, B. Zimdahl, S. Green, J. Lu, J. F. Morales, D. M. Barrett, S. A. Grupp, V. W. Chan, H. Liu, C. Liu, A novel antibody-TCR (AbTCR) platform combines Fab-based antigen recognition with gamma/delta-TCR signaling to facilitate T-cell cytotoxicity with low cytokine release. Cell Discov 4, 62 (2018).
8. K. D. Cummins, S. Gill, Will CAR T cell therapy have a role in AML?Promises and pitfalls. Semin Hematol 56, 155-163 (2019).
9. F. Marofi, H. S. Rahman, Z. M. J. Al-Obaidi, A. T. Jalil, W. K. Abdelbasset, W. Suksatan, A. E. Dorofeev, N. Shomali, M. S. Chartrand, Y. Pathak, A. Hassanzadeh, B. Baradaran, M. Ahmadi, H. Saeedi, S. Tahmasebi, M. Jarahian, Novel CAR T therapy is a ray of hope in the treatment of seriously ill AML patients. Stem Cell Res Ther 12, 465 (2021).
10. Y. Akatsuka, TCR-Like CAR-T Cells Targeting MHC-Bound Minor Histocompatibility Antigens. Front Immunol 11, 257 (2020).
11. T. Dao, S. Yan, N. Veomett, D. Pankov, L. Zhou, T. Korontsvit, A. Scott, J. Whitten, P. Maslak, E. Casey, T. Tan, H. Liu, V. Zakhaleva, M. Curcio, E. Doubrovina, R. J. O'Reilly, C. Liu, D. A. Scheinberg, Targeting the intracellular WT1 oncogene product with a therapeutic human antibody. Sci Transl Med 5, 176ra133 (2013).
12. T. Dao, D. Pankov, A. Scott, T. Korontsvit, V. Zakhaleva, Y. Xu, J. Xiang, S. Yan, M. D. de Morais Guerreiro, N. Veomett, L. Dubrovsky, M. Curcio, E. Doubrovina, V. Ponomarev, C. Liu, R. J. O'Reilly, D. A. Scheinberg, Therapeutic bispecific T-cell engager antibody targeting the intracellular oncoprotein WT1. Nat Biotechnol 33, 1079-1086 (2015).
13. H. Sugiyama, WT1 (Wilms' tumor gene 1): biology and cancer immunotherapy. Jpn J Clin Oncol 40, 377-387 (2010).
14. M. A. Cheever, J. P. Allison, A. S. Ferris, O. J. Finn, B. M. Hastings, T. T. Hecht, I. Mellman, S. A. Prindiville, J. L. Viner, L. M. Weiner, L. M. Matrisian, The prioritization of cancer antigens: a national cancer institute pilot project for the acceleration of translational research. Clin Cancer Res 15, 5323-5337 (2009).
15. C. Augsberger, G. Hanel, W. Xu, V. Pulko, L. J. Hanisch, A. Augustin, J. Challier, K. Hunt, B. Vick, P. E. Rovatti, C. Krupka, M. Rothe, A. Schonle, J. Sam, E. Lezan, A. Ducret, D. Ortiz-Franyuti, A. C. Walz, J. Benz, A. Bujotzek, F. S. Lichtenegger, C. Gassner, A. Carpy, V. Lyamichev, J. Patel, N. Konstandin, A. Tunger, M. Schmitz, M. von Bergwelt-Baildon, K. Spiekermann, L. Vago, I. Jeremias, E. Marrer-Berger, P. Umana, C. Klein, M. Subklewe, Targeting intracellular WT1 in AML with a novel RMF-peptide-MHC-specific T-cell bispecific antibody. Blood 138, 2655-2669 (2021).
16. A. G. Chapuis, D. N. Egan, M. Bar, T. M. Schmitt, M. S. McAfee, K. G. Paulson, V. Voillet, R. Gottardo, G. B. Ragnarsson, M. Bleakley, C. C. Yeung, P. Muhlhauser, H. N. Nguyen, L. A. Kropp, L. Castelli, F. Wagener, D. Hunter, M. Lindberg, K. Cohen, A. Seese, M. J. McElrath, N. Duerkopp, T. A. Gooley, P. D. Greenberg, T cell receptor gene therapy targeting WT1 prevents acute myeloid leukemia relapse post-transplant. Nat Med 25, 1064-1072 (2019).
17. S. Rafiq, T. J. Purdon, A. F. Daniyan, M. Koneru, T. Dao, C. Liu, D. A. Scheinberg, R. J. Brentjens, Optimized T-cell receptor-mimic chimeric antigen receptor T cells directed toward the intracellular Wilms Tumor 1 antigen. Leukemia 31, 1788-1797 (2017).
18. R. J. May, T. Dao, J. Pinilla-Ibarz, T. Korontsvit, V. Zakhaleva, R. H. Zhang, P. Maslak, D. A. Scheinberg, Peptide epitopes from the Wilms' tumor 1 oncoprotein stimulate CD4+ and CD8+T cells that recognize and kill human malignant mesothelioma tumor cells. Clin Cancer Res 13, 4547-4555 (2007).
19. P. G. Maslak, T. Dao, M. Gomez, S. Chanel, J. Packin, T. Korontsvit, V. Zakhaleva, J. Pinilla-Ibarz, E. Berman, D. A. Scheinberg, A pilot vaccination trial of synthetic analog peptides derived from the BCR-ABL breakpoints in CML patients with minimal disease. Leukemia 22, 1613-1616 (2008).
20. N. Ataie, J. Xiang, N. Cheng, E. J. Brea, W. Lu, D. A. Scheinberg, C. Liu, H. L. Ng, Structure of a TCR-Mimic Antibody with Target Predicts Pharmacogenetics. J Mol Biol 428, 194-205 (2016).
21. C. J. Holland, R. M. Crean, J. M. Pentier, B. de Wet, A. Lloyd, V. Srikannathasan, N. Lissin, K. A. Lloyd, T. H. Blicher, P. J. Conroy, M. Hock, R. J. Pengelly, T. E. Spinner, B. Cameron, E. A. Potter, A. Jeyanthan, P. E. Molloy, M. Sami, M. Aleksic, N. Liddy, R. A. Robinson, S. Harper, M. Lepore, C. R. Pudney, M. W. van der Kamp, P. J. Rizkallah, B. K. Jakobsen, A. Vuidepot, D. K. Cole, Specificity of bispecific T cell receptors and antibodies targeting peptide-HLA. J Clin Invest 130, 2673-2688 (2020).
22. D. A. Scheinberg, M. Tanimoto, S. McKenzie, A. Strife, L. J. Old, B. D. Clarkson, Monoclonal antibody M195: a diagnostic marker for acute myelogenous leukemia. Leukemia 3, 440-445 (1989).
23. S. J. Thoma, C. P. Lamping, B. L. Ziegler, Phenotype analysis of hematopoietic CD34+ cell populations derived from human umbilical cord blood using flow cytometry and cDNA-polymerase chain reaction. Blood 83, 2103-2114 (1994).
24. L. Gao, I. Bellantuono, A. Elsasser, S. B. Marley, M. Y. Gordon, J. M. Goldman, H. J. Stauss, Selective elimination of leukemic CD34(+) progenitor cells by cytotoxic T lymphocytes specific for WT1. Blood 95, 2198-2203 (2000).
25. V. Leko, S. A. Rosenberg, Identifying and Targeting Human Tumor Antigens for T Cell-Based Immunotherapy of Solid Tumors. Cancer Cell 38, 454-472 (2020).
26. S. Anguille, Y. Willemen, E. Lion, E. L. Smits, Z. N. Berneman, Dendritic cell vaccination in acute myeloid leukemia. Cytotherapy 14, 647-656 (2012).
27. F. P. Tambaro, H. Singh, E. Jones, M. Rytting, K. M. Mahadeo, P. Thompson, N. Daver, C. DiNardo, T. Kadia, G. Garcia-Manero, T. Chan, R. R. Shah, W. G. Wierda, Autologous CD33-CAR-T cells for treatment of relapsed/refractory acute myelogenous leukemia. Leukemia 35, 3282-3286 (2021).
28. A. Ehninger, M. Kramer, C. Röllig, C. Thiede, M. Bornhauser, M. von Bonin, M. Wermke, A. Feldmann, M. Bachmann, G. Ehninger, U. Oelschlägel, Distribution and levels of cell surface expression of CD33 and CD123 in acute myeloid leukemia. Blood Cancer J 4, e218 (2014).
29. J. D. Griffin, D. Linch, K. Sabbath, P. Larcom, S. F. Schlossman, A monoclonal antibody reactive with normal and leukemic human myeloid progenitor cells. Leuk Res 8, 521-534 (1984).
30. S. Zumerle, B. Molon, A. Viola, Membrane Rafts in T Cell Activation: A Spotlight on CD28 Costimulation. Front Immunol 8, 1467 (2017).
31. A. K. Sewell, Why must T cells be cross-reactive?Nat Rev Immunol 12, 669-677 (2012).
32. A. Y. Chang, R. S. Gejman, E. J. Brea, C. Y. Oh, M. D. Mathias, D. Pankov, E. Casey, T. Dao, D. A. Scheinberg, Opportunities and challenges for TCR mimic antibodies in cancer therapy. Expert Opin Biol Ther 16, 979-987 (2016).
33. D. Mason, A very high level of crossreactivity is an essential feature of the T-cell receptor. Immunol Today 19, 395-404 (1998).
34. P. He, H. Liu, B. Zimdahl, J. Wang, M. Luo, Q. Chang, F. Tian, F. Ni, D. Yu, H. Liu, L. Chen, H. Wang, M. Zhang, S. A. Grupp, C. Liu, A novel antibody-TCR (AbTCR) T-cell therapy is safe and effective against CD19-positive relapsed/refractory B-cell lymphoma. J Cancer Res Clin Oncol, (2022).
35. S. L. Maude, N. Frey, P. A. Shaw, R. Aplenc, D. M. Barrett, N. J. Bunin, A. Chew, V. E. Gonzalez, Z. Zheng, S. F. Lacey, Y. D. Mahnke, J. J. Melenhorst, S. R. Rheingold, A. Shen, D. T. Teachey, B. L. Levine, C. H. June, D. L. Porter, S. A. Grupp, Chimeric antigen receptor T cells for sustained remissions in leukemia. N Engl J Med 371, 1507-1517 (2014).
36. M. L. Davila, R. J. Brentjens, CD19-Targeted CAR T cells as novel cancer immunotherapy for relapsed or refractory B-cell acute lymphoblastic leukemia. Clin Adv Hematol Oncol 14, 802-808 (2016).
37. A. Y. Chang, T. Dao, R. S. Gejman, C. A. Jarvis, A. Scott, L. Dubrovsky, M. D. Mathias, T. Korontsvit, V. Zakhaleva, M. Curcio, R. C. Hendrickson, C. Liu, D. A. Scheinberg, A therapeutic T cell receptor mimic antibody targets tumor-associated PRAME peptide/HLA-I antigens. J Cin Invest 127, 2705-2718 (2017).
38. O. O. Yeku, T. D. Rao, I. Laster, A. Kononenko, T. J. Purdon, P. Wang, Z. Cui, H. Liu, R. J. Brentjens, D. Spriggs, Bispecific T-Cell Engaging Antibodies Against MUC16 Demonstrate Efficacy Against Ovarian Cancer in Monotherapy and in Combination With PD-1 and VEGF Inhibition. Front Immunol 12, 663379 (2021).
39. A. B. Riemer, D. B. Keskin, G. Zhang, M. Handley, K. S. Anderson, V. Brusic, B. Reinhold, E. L. Reinherz, A conserved E7-derived cytotoxic T lymphocyte epitope expressed on human papillomavirus 16-transformed HLA-A2+ epithelial cancers. J Biol Chem 285, 29608-29622 (2010).

1. An anti-WTMC (WT1/major histocompatibility class I protein complex) construct comprising an antibody moiety that specifically binds to a complex comprising WT1 peptide (WT1-RMF) and a major histocompatibility (MHC) class I protein, wherein the WT1-RMF comprises the amino acid sequence of RMFPNAPYL (SEQ ID NO: 113), and wherein the antibody moiety specifically recognizes amino acid residues FPNA (SEQ ID NO: 349) of SEQ ID NO: 113t optionally
wherein the MHC class I protein is ILA-A*02:01, or wherein the antibody moiety comprises:
(i) a V_{H} comprising a HC-CDR1 comprising the amino acid sequence of any one of SEQ ID NOs: 1, 7, 13, 19, 25, 31, 37, 43, 49, 55, 61, 67, 73, or 79, or a variant thereof comprising up to about 3 amino acid substitutions, an HC-CDR2 comprising the amino acid sequence of any one of SEQ ID NOs: 2, 8, 14, 20, 26, 32, 38, 44, 50, 56, 62, 68, 74, or 80, or a variant thereof comprising up to about 3 amino acid substitutions, and an HC-CDR3 comprising the amino acid sequence of any one of SEQ ID NOs: 3, 9, 15, 21, 27, 33, 39, 45, 51, 57, 63, 69, 75 or 81, or a variant thereof comprising up to about 3 amino acid substitutions; and
(ii) a V_{L} comprising a LC-CDR1 comprising the amino acid sequence of any one of SEQ ID NOs: 4, 10, 16, 22, 28, 34, 40, 46, 52, 58, 64, 70, 76, or 82; or a variant thereof comprising up to about 3 amino acid substitutions, an LC-CDR2 comprising the amino acid sequence of any one of SEQ ID NOs: 5, 11, 17, 23, 29, 35, 41, 47, 53, 59, 65, 71, 77, or 83, or a variant thereof comprising about 2 amino acid substitutions, and an LC-CDR3 comprising the amino acid sequence of any one of SEQ ID NOs: 6, 12, 18, 24, 30, 36, 42, 48, 54, 60, 66, 72, 78 or 84, or a variant thereof comprising up to about 3 amino acid substitutions;
or wherein the antibody moiety comprises:
(iii) a V_{H} comprising an HC-CDR1 comprising the amino acid sequence of any one of SEQ ID NOs: 1, 7, 13, 19, 25, 31, 37, 43, 49, 55, 61, 67, 73, or 79, an HC-CDR2 comprising the amino acid sequence of any one of SEQ ID NOs: 2, 8, 14, 20, 26, 32, 38, 44, 50, 56, 62, 68, 74, or 80, and an HC-CDR3 comprising the amino acid sequence of any one of SEQ ID NOs: 3, 9, 15, 21, 27, 33, 39, 45, 51, 57, 63, 69, 75 or 81; and
(iv) a V_{L} comprising an LC-CDR1 comprising the amino acid sequence of any one of SEQ ID NOs: 4, 10, 16, 22, 28, 34, 40, 46, 52, 58, 64, 70, 76, or 82, an LC-CDR2 comprising the amino acid sequence of any one of SEQ ID NOs: 5, 11, 17, 23, 29, 35, 41, 47, 53, 59, 65, 71, 77, or 83, and an LC-CDR3 comprising the amino acid sequence of any one of SEQ ID NOs: 6, 12, 18, 24, 30, 36, 42, 48, 54, 60, 66, 71, 78 or 84.

2. (canceled)

3. (canceled)

4. (canceled)

5. An anti-WTMC construct comprising an antibody moiety that comprises:
(i) a heavy chain immunoglobulin variable domain (V_{H}) comprising a heavy chain complementarity determining region (HC-CDR) 1 sequence, a HC-CDR2 sequence, and a HC-CDR3 sequence of the V_{H} sequence of SEQ ID NO: 85, and a light chain immunoglobulin variable domain (V_{L}) comprising a light chain complementarity determining region (LC-CDR) 1 sequence, a LC-CDR2 sequence, and a LC-CDR3 sequence of the V_{L} sequence SEQ ID NO: 86; or
(ii) a V_{H} comprising a HC-CDR1 sequence, a HC-CDR2 sequence, and a HC-CDR3 sequence of the V_{H} sequence of SEQ ID NO: 87, and a V_{L} comprising a LC-CDR1 sequence, a LC-CDR2 sequence, and a LC-CDR3 sequence of the V_{L} sequence SEQ ID NO: 88; or
(iii) a V_{H} comprising a HC-CDR1 sequence, a HC-CDR2 sequence, and a HC-CDR3 sequence of the V_{H} sequence of SEQ ID NO: 89, and a V_{L} comprising a LC-CDR1 sequence, a LC-CDR2 sequence, and a LC-CDR3 sequence of the V_{L} sequence SEQ ID NO: 90; or
(iv) a V_{H} comprising a HC-CDR1 sequence, a HC-CDR2 sequence, and a HC-CDR3 sequence of the V_{H} sequence of SEQ ID NO: 91, and a V_{L} comprising a LC-CDR1 sequence, a LC-CDR2 sequence, and a LC-CDR3 sequence of the V_{L} sequence SEQ ID NO: 92; or
(v) a V_{H} comprising a HC-CDR1 sequence, a HC-CDR2 sequence, and a HC-CDR3 sequence of the V_{H} sequence of SEQ ID NO: 93, and a V_{L} comprising a LC-CDR1 sequence, a LC-CDR2 sequence, and a LC-CDR3 sequence of the V_{L} sequence SEQ ID NO: 94; or
(vi) a V_{H} comprising a HC-CDR1 sequence, a HC-CDR2 sequence, and a HC-CDR3 sequence of the V_{H} sequence of SEQ ID NO: 95, and a V_{L} comprising a LC-CDR1 sequence, a LC-CDR2 sequence, and a LC-CDR3 sequence of the V_{L} sequence SEQ ID NO: 96; or
(vii) a V_{H} comprising a HC-CDR1 sequence, a HC-CDR2 sequence, and a HC-CDR3 sequence of the V_{H} sequence of SEQ ID NO: 97, and a V_{L} comprising a LC-CDR1 sequence, a LC-CDR2 sequence, and a LC-CDR3 sequence of the V_{L} sequence SEQ ID NO: 98; or
(viii) a V_{H} comprising a HC-CDR1 sequence, a HC-CDR2 sequence, and a HC-CDR3 sequence of the V_{H} sequence of SEQ ID NO: 99, and a V_{L} comprising a LC-CDR1 sequence, a LC-CDR2 sequence, and a LC-CDR3 sequence of the V_{L} sequence SEQ ID NO: 100; or
(ix) a V_{H} comprising a HC-CDR1 sequence, a HC-CDR2 sequence, and a HC-CDR3 sequence of the V_{H} sequence of SEQ ID NO: 101, and a V_{L} comprising a LC-CDR1 sequence, a LC-CDR2 sequence, and a LC-CDR3 sequence of the V_{L} sequence SEQ ID NO: 102; or
(x) a V_{H} comprising a HC-CDR1 sequence, a HC-CDR2 sequence, and a HC-CDR3 sequence of the V_{H} sequence of SEQ ID NO: 103, and a V_{L} comprising a LC-CDR1 sequence, a LC-CDR2 sequence, and a LC-CDR3 sequence of the V_{L} sequence SEQ ID NO: 104; or
(xi) a V_{H} comprising a HC-CDR1 sequence, a HC-CDR2 sequence, and a HC-CDR3 sequence of the V_{H} sequence of SEQ ID NO: 105, and a V_{L} comprising a LC-CDR1 sequence, a LC-CDR2 sequence, and a LC-CDR3 sequence of the V_{L} sequence SEQ ID NO: 106; or
(xii) a V_{H} comprising a HC-CDR1 sequence, a HC-CDR2 sequence, and a HC-CDR3 sequence of the V_{H} sequence of SEQ ID NO: 107, and a V_{L} comprising a LC-CDR1 sequence, a LC-CDR2 sequence, and a LC-CDR3 sequence of the V_{L} sequence SEQ ID NO: 108; or
(xiii) a V_{H} comprising a HC-CDR1 sequence, a HC-CDR2 sequence, and a HC-CDR3 sequence of the V_{H} sequence of SEQ ID NO: 109, and a V_{L} comprising a LC-CDR1 sequence, a LC-CDR2 sequence, and a LC-CDR3 sequence of the V_{L} sequence SEQ ID NO: 110; or
(xiv) a V_{H} comprising a HC-CDR1 sequence, a HC-CDR2 sequence, and a HC-CDR3 sequence of the V_{H} sequence of SEQ ID NO: 111, and a V_{L} comprising a LC-CDR1 sequence, a LC-CDR2 sequence, and a LC-CDR3 sequence of the V_{L} sequence SEQ ID NO: 112;
optionally wherein the construct specifically binds to a complex comprising WT1-RMF and an MHC class I protein, wherein the WT1-RMF comprises the amino acid sequence of SEQ ID NO: 113, or wherein the MHC class I protein is HLA-A*02:01.

6. (canceled)

7. The anti-WTMC construct of claim 1, wherein the antibody moiety comprises:
1) i) a V_{H} comprising an HC-CDR1 comprising the amino acid sequence of SEQ ID NO: 1, an HC-CDR2 comprising the amino acid sequence of SEQ ID NO: 2, and an HC-CDR3 comprising the amino acid sequence of SEQ ID NO: 3; and ii) a V_{L} comprising an LC-CDR1 comprising the amino acid sequence of SEQ ID NO: 4, an LC-CDR2 comprising the amino acid sequence of SEQ ID NO: 5, and an LC-CDR3 comprising the amino acid sequence of SEQ ID NO: 6; or
2) i) a V_{H} comprising an HC-CDR1 comprising the amino acid sequence of SEQ ID NO: 7, an HC-CDR2 comprising the amino acid sequence of SEQ ID NO: 8, and an HC-CDR3 comprising the amino acid sequence of SEQ ID NO: 9; and ii) a V_{L} comprising an LC-CDR1 comprising the amino acid sequence of SEQ ID NO: 10, an LC-CDR2 comprising the amino acid sequence of SEQ ID NO: 11, and an LC-CDR3 comprising the amino acid sequence of SEQ ID NO: 12; or
3) i) a V_{H} comprising an HC-CDR1 comprising the amino acid sequence of SEQ ID NO: 13, an HC-CDR2 comprising the amino acid sequence of SEQ ID NO: 14, and an HC-CDR3 comprising the amino acid sequence of SEQ ID NO: 15; and ii) a light chain variable domain (V_{L}) comprising an LC-CDR1 comprising the amino acid sequence of SEQ ID NO: 16, an LC-CDR2 comprising the amino acid sequence of SEQ ID NO: 17, and an LC-CDR3 comprising the amino acid sequence of SEQ ID NO: 18; or
4) i) a V_{H} comprising an HC-CDR1 comprising the amino acid sequence of SEQ ID NO: 19, an HC-CDR2 comprising the amino acid sequence of SEQ ID NO: 20, and an HC-CDR3 comprising the amino acid sequence of SEQ ID NO: 21; and ii) a light chain variable domain (V_{L}) comprising an LC-CDR1 comprising the amino acid sequence of SEQ ID NO: 22, an LC-CDR2 comprising the amino acid sequence of SEQ ID NO: 23, and an LC-CDR3 comprising the amino acid sequence of SEQ ID NO: 24; or
5) i) a V_{H} comprising an HC-CDR1 comprising the amino acid sequence of SEQ ID NO: 25, an HC-CDR2 comprising the amino acid sequence of SEQ ID NO: 26, and an HC-CDR3 comprising the amino acid sequence of SEQ ID NO: 27; and ii) a light chain variable domain (V_{L}) comprising an LC-CDR1 comprising the amino acid sequence of SEQ ID NO: 28, an LC-CDR2 comprising the amino acid sequence of SEQ ID NO: 29, and an LC-CDR3 comprising the amino acid sequence of SEQ ID NO: 30; or
6) i) a V_{H} comprising an HC-CDR1 comprising the amino acid sequence of SEQ ID NO: 31, an HC-CDR2 comprising the amino acid sequence of SEQ ID NO: 32, and an HC-CDR3 comprising the amino acid sequence of SEQ ID NO: 33; and ii) a light chain variable domain (V_{L}) comprising an LC-CDR1 comprising the amino acid sequence of SEQ ID NO: 34, an LC-CDR2 comprising the amino acid sequence of SEQ ID NO: 35, and an LC-CDR3 comprising the amino acid sequence of SEQ ID NO: 36; or
7) i) a V_{H} comprising an HC-CDR1 comprising the amino acid sequence of SEQ ID NO: 37, an HC-CDR2 comprising the amino acid sequence of SEQ ID NO: 38, and an HC-CDR3 comprising the amino acid sequence of SEQ ID NO: 39; and ii) a light chain variable domain (V_{L}) comprising an LC-CDR1 comprising the amino acid sequence of SEQ ID NO: 40, an LC-CDR2 comprising the amino acid sequence of SEQ ID NO: 41, and an LC-CDR3 comprising the amino acid sequence of SEQ ID NO: 42; or
8) i) a V_{H} comprising an HC-CDR1 comprising the amino acid sequence of SEQ ID NO: 43, an HC-CDR2 comprising the amino acid sequence of SEQ ID NO: 44, and an HC-CDR3 comprising the amino acid sequence of SEQ ID NO: 45; and ii) a light chain variable domain (V_{L}) comprising an LC-CDR1 comprising the amino acid sequence of SEQ ID NO: 46, an LC-CDR2 comprising the amino acid sequence of SEQ ID NO: 47, and an LC-CDR3 comprising the amino acid sequence of SEQ ID NO: 48; or
9) i) a V_{H} comprising an HC-CDR1 comprising the amino acid sequence of SEQ ID NO: 49, an HC-CDR2 comprising the amino acid sequence of SEQ ID NO: 50, and an HC-CDR3 comprising the amino acid sequence of SEQ ID NO: 51; and ii) a light chain variable domain (V_{L}) comprising an LC-CDR1 comprising the amino acid sequence of SEQ ID NO: 52, an LC-CDR2 comprising the amino acid sequence of SEQ ID NO: 53, and an LC-CDR3 comprising the amino acid sequence of SEQ ID NO: 54; or
10)i) a V_{H} comprising an HC-CDR1 comprising the amino acid sequence of SEQ ID NO: 55, an HC-CDR2 comprising the amino acid sequence of SEQ ID NO: 56, and an HC-CDR3 comprising the amino acid sequence of SEQ ID NO: 57; and ii) a light chain variable domain (V_{L}) comprising an LC-CDR1 comprising the amino acid sequence of SEQ ID NO: 58, an LC-CDR2 comprising the amino acid sequence of SEQ ID NO: 59, and an LC-CDR3 comprising the amino acid sequence of SEQ ID NO: 60; or
11)i) a V_{H} comprising an HC-CDR1 comprising the amino acid sequence of SEQ ID NO: 61, an HC-CDR2 comprising the amino acid sequence of SEQ ID NO: 62, and an HC-CDR3 comprising the amino acid sequence of SEQ ID NO: 63; and ii) a light chain variable domain (V_{L}) comprising an LC-CDR1 comprising the amino acid sequence of SEQ ID NO: 64, an LC-CDR2 comprising the amino acid sequence of SEQ ID NO: 65, and an LC-CDR3 comprising the amino acid sequence of SEQ ID NO: 66; or
12)i) a V_{H} comprising an HC-CDR1 comprising the amino acid sequence of SEQ ID NO: 67, an HC-CDR2 comprising the amino acid sequence of SEQ ID NO: 68, and an HC-CDR3 comprising the amino acid sequence of SEQ ID NO: 69; and ii) a light chain variable domain (V_{L}) comprising an LC-CDR1 comprising the amino acid sequence of SEQ ID NO: 70, an LC-CDR2 comprising the amino acid sequence of SEQ ID NO: 71, and an LC-CDR3 comprising the amino acid sequence of SEQ ID NO: 72; or
13)i) a V_{H} comprising an HC-CDR1 comprising the amino acid sequence of SEQ ID NO: 73, an HC-CDR2 comprising the amino acid sequence of SEQ ID NO: 74, and an HC-CDR3 comprising the amino acid sequence of SEQ ID NO: 75; and ii) a light chain variable domain (V_{L}) comprising an LC-CDR1 comprising the amino acid sequence of SEQ ID NO: 76, an LC-CDR2 comprising the amino acid sequence of SEQ ID NO: 77, and an LC-CDR3 comprising the amino acid sequence of SEQ ID NO: 78; or
14)i) a V_{H} comprising an HC-CDR1 comprising the amino acid sequence of SEQ ID NO: 79, an HC-CDR2 comprising the amino acid sequence of SEQ ID NO: 80, and an HC-CDR3 comprising the amino acid sequence of SEQ ID NO: 81; and ii) a light chain variable domain (V_{L}) comprising an LC-CDR1 comprising the amino acid sequence of SEQ ID NO: 82, an LC-CDR2 comprising the amino acid sequence of SEQ ID NO: 83, and an LC-CDR3 comprising the amino acid sequence of SEQ ID NO: 84; or
wherein the antibody moiety comprises:
15)i) a V_{H} comprising the amino acid sequence of SEQ ID NO: 85, or a variant thereof having at least about 95% sequence identity to the amino acid sequence of SEQ ID NO: 85; and ii) a V_{L} comprising the amino acid sequence of SEQ ID NO: 86, or a variant thereof having at least about 95% sequence identity to the amino acid sequence of SEQ ID NO: 86; or
16)i) a V_{H} comprising the amino acid sequence of SEQ ID NO: 87, or a variant thereof having at least about 95% sequence identity to the amino acid sequence of SEQ ID NO: 87; and ii) a V_{L} comprising the amino acid sequence of SEQ ID NO: 88, or a variant thereof having at least about 95% sequence identity to the amino acid sequence of SEQ ID NO: 88; or
17)i) a V_{H} comprising the amino acid sequence of SEQ ID NO: 89, or a variant thereof having at least about 95% sequence identity to the amino acid sequence of SEQ ID NO: 89; and ii) a V_{L} comprising the amino acid sequence of SEQ ID NO: 90, or a variant thereof having at least about 95% sequence identity to the amino acid sequence of SEQ ID NO: 90; or
18)i) a V_{H} comprising the amino acid sequence of SEQ ID NO: 91, or a variant thereof having at least about 95% sequence identity to the amino acid sequence of SEQ ID NO: 91; and ii) a V_{L} comprising the amino acid sequence of SEQ ID NO: 92, or a variant thereof having at least about 95% sequence identity to the amino acid sequence of SEQ ID NO: 92; or
19)i) a V_{H} comprising the amino acid sequence of SEQ ID NO: 93, or a variant thereof having at least about 95% sequence identity to the amino acid sequence of SEQ ID NO: 93; and ii) a V_{L} comprising the amino acid sequence of SEQ ID NO: 94, or a variant thereof having at least about 95% sequence identity to the amino acid sequence of SEQ ID NO: 94; or
20)i) a V_{H} comprising the amino acid sequence of SEQ ID NO: 95, or a variant thereof having at least about 95% sequence identity to the amino acid sequence of SEQ ID NO: 95; and ii) a V_{L} comprising the amino acid sequence of SEQ ID NO: 96, or a variant thereof having at least about 95% sequence identity to the amino acid sequence of SEQ ID NO: 96; or
21)i) a V_{H} comprising the amino acid sequence of SEQ ID NO: 97, or a variant thereof having at least about 95% sequence identity to the amino acid sequence of SEQ ID NO: 97; and ii) a V_{L} comprising the amino acid sequence of SEQ ID NO: 98, or a variant thereof having at least about 95% sequence identity to the amino acid sequence of SEQ ID NO: 98; or
22)i) a V_{H} comprising the amino acid sequence of SEQ ID NO: 99, or a variant thereof having at least about 95% sequence identity to the amino acid sequence of SEQ ID NO: 99; and ii) a V_{L} comprising the amino acid sequence of SEQ ID NO: 100, or a variant thereof having at least about 95% sequence identity to the amino acid sequence of SEQ ID NO: 100; or
23)i) a V_{H} comprising the amino acid sequence of SEQ ID NO: 101, or a variant thereof having at least about 95% sequence identity to the amino acid sequence of SEQ ID NO: 101; and ii) a V_{L} comprising the amino acid sequence of SEQ ID NO: 102, or a variant thereof having at least about 95% sequence identity to the amino acid sequence of SEQ ID NO: 102; or
24)i) a V_{H} comprising the amino acid sequence of SEQ ID NO: 103, or a variant thereof having at least about 95% sequence identity to the amino acid sequence of SEQ ID NO: 103; and ii) a V_{L} comprising the amino acid sequence of SEQ ID NO: 104, or a variant thereof having at least about 95% sequence identity to the amino acid sequence of SEQ ID NO: 104; or
25)i) a V_{H} comprising the amino acid sequence of SEQ ID NO: 105, or a variant thereof having at least about 95% sequence identity to the amino acid sequence of SEQ ID NO: 105; and ii) a V_{L} comprising the amino acid sequence of SEQ ID NO: 106, or a variant thereof having at least about 95% sequence identity to the amino acid sequence of SEQ ID NO: 106; or
26)i) a V_{H} comprising the amino acid sequence of SEQ ID NO: 107, or a variant thereof having at least about 95% sequence identity to the amino acid sequence of SEQ ID NO: 107; and ii) a V_{L} comprising the amino acid sequence of SEQ ID NO: 108, or a variant thereof having at least about 95% sequence identity to the amino acid sequence of SEQ ID NO: 108; or
27)i) a V_{H} comprising the amino acid sequence of SEQ ID NO: 109, or a variant thereof having at least about 95% sequence identity to the amino acid sequence of SEQ ID NO: 109; and ii) a V_{L} comprising the amino acid sequence of SEQ ID NO: 110, or a variant thereof having at least about 95% sequence identity to the amino acid sequence of SEQ ID NO: 110; or
28)i) a V_{H} comprising the amino acid sequence of SEQ ID NO: 111, or a variant thereof having at least about 95% sequence identity to the amino acid sequence of SEQ ID NO: 111; and ii) a V_{L} comprising the amino acid sequence of SEQ ID NO: 112, or a variant thereof having at least about 95% sequence identity to the amino acid sequence of SEQ ID NO: 112; or
wherein the antibody moiety comprises:
29)i) a V_{H} comprising the amino acid sequence of SEQ ID NO: 85; and ii) a V_{L} comprising the amino acid sequence of SEQ ID NO: 86; or
30)i) a V_{H} comprising the amino acid sequence of SEQ ID NO: 87; and ii) a V_{L} comprising the amino acid sequence of SEQ ID NO: 88; or
31)i) a V_{H} comprising the amino acid sequence of SEQ ID NO: 89; and ii) a V_{L} comprising the amino acid sequence of SEQ ID NO: 90; or
32)i) a V_{H} comprising the amino acid sequence of SEQ ID NO: 91; and ii) a V_{L} comprising the amino acid sequence of SEQ ID NO: 92; or
33)i) a V_{H} comprising the amino acid sequence of SEQ ID NO: 93; and ii) a V_{L} comprising the amino acid sequence of SEQ ID NO: 94; or
34)i) a V_{H} comprising the amino acid sequence of SEQ ID NO: 95; and ii) a V_{L} comprising the amino acid sequence of SEQ ID NO: 96; or
35)i) a V_{H} comprising the amino acid sequence of SEQ ID NO: 97; and ii) a V_{L} comprising the amino acid sequence of SEQ ID NO: 98; or
36)i) a V_{H} comprising the amino acid sequence of SEQ ID NO: 99; and ii) a V_{L} comprising the amino acid sequence of SEQ ID NO: 100; or
37)i) a V_{H} comprising the amino acid sequence of SEQ ID NO: 101; and ii) a V_{L} comprising the amino acid sequence of SEQ ID NO: 102; or
38)i) a V_{H} comprising the amino acid sequence of SEQ ID NO: 103; and ii) a V_{L} comprising the amino acid sequence of SEQ ID NO: 104; or
39)i) a V_{H} comprising the amino acid sequence of SEQ ID NO: 105; and ii) a V_{L} comprising the amino acid sequence of SEQ ID NO: 106; or
40)i) a V_{H} comprising the amino acid sequence of SEQ ID NO: 107; and ii) a V_{L} comprising the amino acid sequence of SEQ ID NO: 108; or
41)i) a V_{H} comprising the amino acid sequence of SEQ ID NO: 109; and ii) a V_{L} comprising the amino acid sequence of SEQ ID NO: 110; or
42)i) a V_{H} comprising the amino acid sequence of SEQ ID NO: 111; and ii) a V_{L} comprising the amino acid sequence of SEQ ID NO: 112.

8. (canceled)

9. (canceled)

10. The anti-WTMC construct of claim 1, wherein the antibody moiety is chimeric, humanized, partially human, fully human, or semi-synthetic or
wherein the antibody moiety is a full-length antibody, a Fab, a Fab′, a F(ab′)₂, an Fv; or a single chain Fv (scFv): optionally wherein the antibody moiety is an scFv, optionally wherein the scFv comprises the amino acid sequence of any one of SEQ ID NOs: 335-348: optionally wherein the antibody moiety is a Fab or Fab′; or
wherein the antibody moiety specifically recognizing WTMC is fused to an Fe fragment, optionally via a linker, optionally wherein the Fe fragment is an IgG Fc fragment, optionally wherein the IgG is an IgG1, IgG2, IgG3, or IgG4; or
wherein the anti-WTMC construct is a full-length antibody; or
wherein the anti-WTMC construct is monospecific; or
wherein the anti-WTMC construct is multispecific or bispecific, optionally wherein the anti-WTMC construct is a tandem scFv, a diabody (Db), a single chain diabody (scDb) a dual-affinity retargeting (DART) antibody, a F(ab′)₂, a dual variable domain (DVD) antibody, a knob-into-hole (KiH) antibody, a dock and lock (DNL) antibody, a chemically cross-linked antibody, a heteromultimeric antibody; or a heteroconjugate antibody, optionally wherein wherein the anti-WTMC construct is a tandem scFv comprising two scFvs linked by a peptide linker, optionally wherein the anti-WTMC construct further comprises a second antibody moiety specifically recognizing a second antigen, optionally wherein the second antigen is an antigen on the surface of a T cell, optionally wherein the second antigen is selected from the group consisting of CD3, CD3γ, CD3δ, CD3ε, CD3ζ, CD28, OX40, GITR, CD137, CD27, CD40L, and HVEM, optionally wherein the second antigen is CD3ε, optionally wherein the anti-WTMC construct is a tandem scFv comprising an N-terminal scFv specifically recognizing WTMC and a C-terminal scFv specifically recognizing CD3ε, optionally wherein the T cell is selected from the group consisting of a cytotoxic T cell, a helper T cell, and a natural killer T (NKT) cell, optionally wherein the expression of the anti-WTMC construct is induced by the activation of an engineered T cell or natural killer (NK) cell, optionally wherein the engineered T cell or NK cell is a T cell or NK cell comprising a chimeric antigen receptor (CAR), optionally wherein the engineered T cell or NK cell is a T cell or NK cell comprising a chimeric antibody-T cell receptor construct (caTCR), optionally wherein the second antigen is an antigen on the surface of a B cell, a natural killer cell, a dendritic cell, a macrophage, a monocyte, or a neutrophil; or
wherein the anti-WTMC construct is a conjugate comprising the antibody moiety and an effector molecule, optionally wherein the effector molecule is a therapeutic agent selected from the group consisting of a drug, a toxin, a radioisotope, a protein, a peptide, and a nucleic acid, optionally wherein the effector molecule is a therapeutic agent, and wherein the therapeutic agent is a drug or a toxin, optionally wherein the effector molecule is a detectable label.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. The anti-WTMC construct of claim 1, wherein the anti-WTMC construct is a CAR comprising:
(a) an extracellular domain comprising the antibody moiety;
(b) a transmembrane domain; and
(c) an intracellular signaling domain;
optionally wherein the intracellular signaling domain comprises a primary immune cell signaling sequence derived from CD3ζ, TCRζ, FcRγ, FcRβ, CD3γ, CD3δ, CD3ε, CD5, CD22, CD79a, CD79b, or CD66d, optionally wherein the intracellular signaling domain further comprises a costimulatory signaling sequence derived from CD28, CD30, 4-1BB, DAP10, ICOS, or OX40, optionally wherein the intracellular signaling domain comprises a CD3ζ intracellular signaling sequence and a CD30 intracellular signaling sequence, optionally wherein the intracellular signaling domain comprises a CD3ζ intracellular signaling sequence and a CD30 intracellular signaling sequence, optionally wherein the intracellular signaling domain comprises a CD3ζ intracellular signaling sequence and a 4-1BB intracellular signaling sequence, optionally wherein the intracellular signaling domain further comprise a costimulatory signaling sequence derived from CD28, CD30, 4-1BB, DAP10, ICOS; or OX40; or
wherein the anti-WTMC construct is a caTCR comprising:
(a) an extracellular domain comprising the antibody moiety; and
(b) a T cell receptor module (TCRM) comprising a first TCR domain (TCRD) comprising a first TCR transmembrane domain (TCR-TM) and a second TCRD comprising a second TCR-TM, wherein the TCRM is capable of associating with at least one TCR-associated signaling molecule: optionally
wherein the caTCR lacks a functional primary immune cell signaling domain of a TCR-associated T cell activation molecule selected from the group consisting of CD3ζ (TCRζ), FcRγ, FcRβ, CD3γ, CD3δ, CD3ε, CD5, CD22, CD79a, CD79b, and CD66d, optionally wherein the caTCR lacks any primary immune cell signaling sequences; or wherein the first TCR-TM is derived from one of the transmembrane domains of a first naturally occurring TCR and the second TCR-TM is derived from the other transmembrane domain of the first naturally occurring TCR; or wherein the at least one of the TCR-TMs is non-naturally occurring, or wherein the TCRM comprising the at least one non-naturally occurring TCR-TM allows for enhanced association of the at least one TCR-associated signaling molecule as compared to a TCRM comprising the first naturally occurring T cell receptor transmembrane domains, or wherein the first TCR-TM and the second TCR-TM are derived from a γ/δ TCR; or wherein the first TCR-TM and the second TCR-TM are derived from an α/β TCR, or wherein the at least one TCR-associated signaling molecule is selected from the group consisting of CD3δε, CD3γε, and ζζ.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. The anti-WTMC construct of claim 1, wherein the construct is a chimeric signaling receptor (CSR) comprising:
i) a target-binding module comprising the antibody moiety;
ii) a transmembrane module; and
iii) a co-stimulatory immune cell signaling module that is capable of providing a co-stimulatory signal to the effector cell,
and wherein the CSR lacks a functional primary immune cell signaling domain of a TCR-associated T cell activation molecule selected from the group consisting of CD3ζ (TCRζ), FcRγ, FcRβ, CD3γ, CD3δ, CD3ε, CD5, CD22, CD79a, CD79b, and CD66d, optionally wherein the CSR lacks any primary immune cell signaling sequences; or wherein the transmembrane module of the CSR and the co-stimulatory immune cell signaling module of the CSR are from the same molecule; or wherein the transmembrane module of the CSR and the co-stimulatory immune cell signaling module of the CSR are from different molecules; or
wherein the transmembrane module of the CSR comprises one or more transmembrane domains derived from CD28, CD30, CD3ε, CD3ζ, CD45, CD4, CD5, CD8, CD9, CD16, CD22, CD27, CD33, CD37, CD64, CD80, CD86, OX40 (CD134), 4-1BB (CD137), CD154, or ICOS;
or wherein the co-stimulatory immune cell signaling module is derived from the intracellular domain of a co-stimulatory receptor of a TCR, optionally wherein the co-stimulatory receptor is selected from the group consisting of CD28, 4-1BB, OX40, ICOS, CD27, CD30, CD40, and DAP10, optionally wherein the co-stimulatory receptor is CD28, optionally wherein the co-stimulatory receptor is CD30.

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. An isolated nucleic acid or a set of isolated nucleic acids encoding the polypeptide component(s) of the anti-WTMC construct of claim 1, optionally wherein the nucleic acid or set of nucleic acids is comprised in a vector or set of vectors.

63. (canceled)

64. A host cell comprising the anti-WTMC construct of claim 1.

65. A method of producing an anti-WTMC construct, comprising culturing the host cell of claim 64 under conditions where the anti-WTMC construct is expressed, and recovering the anti-WTMC construct produced by the host cell.

66. An effector cell expressing the anti-WTMC constructs of claim 1 optionally wherein the effector cell is an immune cell, optionally wherein the immune cell is a T cell, optionally wherein the T cell is cytotoxic T cell, a helper T cell, a NKT cell, or a suppressor T cell; or wherein the immune cell is a NK cell.

67. An effector cell expressing the anti-WTMC CSR of claim 1, and wherein the effector cell further expresses a CAR comprising:
(a) an extracellular domain that specifically binds a target;
(b) a transmembrane domain; and
(c) an intracellular signaling domain; or
wherein the effector cell further expresses a caTCR comprising:
(a) an extracellular domain that specifically binds a target; and
(b) a T cell receptor module (TCRM) comprising a first TCR domain (TCRD) comprising a first TCR transmembrane domain (TCR-TM) and a second TCRD comprising a second TCR-TM, wherein the TCRM is capable of associating with at least one TCR-associated signaling molecule; or
wherein the effector cell further expresses a TCR that specifically binds a complex comprising a peptide and an MHC protein; optionally
wherein the CAR, caTCR, or TCR specifically binds a WTMC; optionally wherein the CAR, caTCR, or TCR specifically binds a WTMC that is different from the WTMC specifically recognized by the CSR; optionally or wherein the CAR, caTCR, or TCR specifically binds a WTMC that is identical to the WTMC specifically recognized by the CSR; or
wherein the CAR, caTCR, or TCR specifically recognizes a target that is not a WTMC; or
wherein the target specifically recognized by the CAR, caTCR, or TCR is expressed on a cancer cell, optionally wherein the target specifically recognized by the CAR, caTCR, or TCR is expressed on a cancer cell selected from among chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer, and glioblastoma or wherein the effector cell is an immune cell, optionally wherein the immune cell is a T cell, optionally wherein the T cell is cytotoxic T cell, a helper T cell, a NKT cell, or a suppressor T cell or wherein the immune cell is a NK cell.

68. (canceled)

69. (canceled)

70. (canceled)

71. (canceled)

72. (canceled)

73. (canceled)

74. (canceled)

75. (canceled)

76. An effector cell expressing the anti-WTMC caTCR of claim 34, and wherein the effector cell further expresses a CSR comprising:
i) a target-binding domain that specifically binds a target;
ii) a transmembrane module; and
iii) a co-stimulatory immune cell signaling module that is capable of providing a co-stimulatory signal to the effector cell,
wherein the target-binding domain and the co-stimulatory immune cell signaling module are not derived from the same molecule,
and wherein the CSR lacks a functional primary immune cell signaling domain of a TCR-associated T cell activation molecule selected from the group consisting of CD3ζ (TCRζ), FcRγ, FcRβ, CD3γ, CD3δ, CD3ε, CD5, CD22, CD79a, CD79b, and CD66d; optionally wherein the CSR specifically binds a WTMC, optionally wherein the CSR specifically binds a WTMC that is different from the WTMC bound by the caTCR or wherein the CSR specifically binds a WTMC that is identical to the WTMC bound by the caTCR, optionally wherein the CSR specifically binds a target that is not a WTMC, optionally wherein the target bound by the CSR is expressed on a cancer cell, optionally wherein the target bound by the CSR is expressed on a cancer cell selected from the group consisting of chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma; or
wherein the CSR specifically binds CD33, CD371, CD123, or CD15, optionally wherein the CSR specifically binds both CD33 and any one of CD371, CD123, or CD15; or
wherein the CSR specifically binds MUC16 or HER2.

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. An effector cell expressing the anti-WTMC CAR of claim 34 optionally
wherein the effector cell further expresses a CSR comprising:
i) a target-binding domain that specifically binds a target;
ii) a transmembrane module; and
iii) a co-stimulatory immune cell signaling module that is capable of providing a co-stimulatory signal to the effector cell,
wherein the target-binding domain and the co-stimulatory immune cell signaling module are not derived from the same molecule,
and wherein the CSR lacks a functional primary immune cell signaling domain of a TCR-associated T cell activation molecule selected from the group consisting of CD3ζ (TCRζ), FcRγ, FcRβ, CD3γ, CD3δ, CD3ε, CD5, CD22, CD79a, CD79b, and CD66d, optionally
wherein the CSR specifically binds a WTMC, optionally wherein the CSR specifically binds a WTMC that is different from the WTMC bound by the CAR or wherein the CSR specifically binds a WTMC that is identical to the WTMC bound by the CAR; or
wherein the CSR specifically binds a target that is not a WTMC; or wherein the target bound by the CSR is expressed on a cancer cell, optionally wherein the target bound by the CSR is expressed on a cancer cell selected from among chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer and glioblastoma; or
wherein the CSR specifically binds CD33, CD371, CD123, or CD15, optionally wherein the CSR specifically binds both CD33 and any one of CD371, CD123, and CD15; or
wherein the CSR specifically binds HER2 or MUC16.

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. A pharmaceutical composition comprising the anti-WTMC construct of claim 1, and a pharmaceutical acceptable carrier.

105. A kit comprising the anti-WTMC construct of claim 1.

106. A method of detecting WT1-RMF in a sample, comprising contacting the sample with the anti-WTMC construct of claim 10 and detecting the presence of the label.

107. A method of treating an individual having a disease or disorder associated with expression, aberrant expression and/or aberrant activity of WT1, comprising administering to the individual an effective amount of the anti-WTMC construct of claim 1, optionally wherein the method further comprises administering to the individual an additional therapy; or
wherein the disease or disorder associated with expression, aberrant expression, and/or aberrant activity of WT1-RMF is cancer, optionally wherein the cancer is a WT1-positive cancer, optionally wherein the cancer is chronic myelocytic leukemia, multiple myeloma (MM), acute lymphoblastic leukemia (ALL), acute myeloid/myelogenous leukemia (AML), myelodysplastic syndrome (MDS), mesothelioma, ovarian cancer, gastrointestinal cancers, breast cancer, prostate cancer or glioblastoma, optionally wherein the cancer is AML.

108. (canceled)

109. (canceled)

110. A method of diagnosing an individual having a disease or disorder associated with expression, aberrant expression and/or aberrant activity of WT1-RMF, comprising:
I.
administering an effective amount of the anti-WTMC construct of claim 10 to the individual; and
determining the level of the label in the individual, wherein a level of the label above a threshold level indicates that the individual has the disease or disorder associated with expression of WT1-RMF; or
II.
contacting a sample derived from the individual with the anti-WTMC construct of claim 10; and
determining the number of cells bound with the anti-WTMC construct in the sample, wherein a value for the number of cells bound with the anti-WTMC construct above a threshold level indicates that the individual has the disease or disorder associated with expression, aberrant expression, and/or aberrant activity of WT1-RMF.

111. (canceled)

112. (canceled)

113. (canceled)

114. (canceled)

115. (canceled)

116. An anti-WTMC construct comprising an antibody moiety, wherein the antibody moiety comprises:
1) i) a V_{H} comprising the amino acid sequence of SEQ ID NO: 85; and ii) a V_{L} comprising the amino acid sequence of SEQ ID NO: 86;
2) i) a V_{H} comprising the amino acid sequence of SEQ ID NO: 87; and ii) a V_{L} comprising the amino acid sequence of SEQ ID NO: 88;
3) i) a V_{H} comprising the amino acid sequence of SEQ ID NO: 89; and ii) a V_{L} comprising the amino acid sequence of SEQ ID NO: 90;
4) i) a V_{H} comprising the amino acid sequence of SEQ ID NO: 91; and ii) a V_{L} comprising the amino acid sequence of SEQ ID NO: 92; or
5) i) a V_{H} comprising the amino acid sequence of SEQ ID NO: 93; and ii) a V_{L} comprising the amino acid sequence of SEQ ID NO: 94; or
6) i) a V_{H} comprising the amino acid sequence of SEQ ID NO: 95; and ii) a V_{L} comprising the amino acid sequence of SEQ ID NO: 96; or
7) i) a V_{H} comprising the amino acid sequence of SEQ ID NO: 97; and ii) a V_{L} comprising the amino acid sequence of SEQ ID NO: 98; or
8) i) a V_{H} comprising the amino acid sequence of SEQ ID NO: 99; and ii) a V_{L} comprising the amino acid sequence of SEQ ID NO: 100; or
9) i) a V_{H} comprising the amino acid sequence of SEQ ID NO: 101; and ii) a V_{L} comprising the amino acid sequence of SEQ ID NO: 102; or
10)i) a V_{H} comprising the amino acid sequence of SEQ ID NO: 103; and ii) a V_{L} comprising the amino acid sequence of SEQ ID NO: 104; or
11)i) a V_{H} comprising the amino acid sequence of SEQ ID NO: 105; and ii) a V_{L} comprising the amino acid sequence of SEQ ID NO: 106; or
12)i) a V_{H} comprising the amino acid sequence of SEQ ID NO: 107; and ii) a V_{L} comprising the amino acid sequence of SEQ ID NO: 108; or
13)i) a V_{H} comprising the amino acid sequence of SEQ ID NO: 109; and ii) a V_{L} comprising the amino acid sequence of SEQ ID NO: 110; or
14)i) a V_{H} comprising the amino acid sequence of SEQ ID NO: 111; and ii) a V_{L} comprising the amino acid sequence of SEQ ID NO: 112.

117. A method of killing a target cell that expresses a complex comprising WT1-RMF and an MHC class I protein, the method comprising contacting the target cell with an effective amount of the anti-WTMC construct of claim 1, optionally wherein the target cell is a cancer cell, optionally wherein the cancer cell is a leukemia, optionally wherein the cancer cell is a solid tumor.

118. (canceled)

119. (canceled)

120. (canceled)


SEQUENCE LISTING (349 sequences)

SEQ ID NO: 1 - protein, 8 aa, synthetic construct
GYNFTSYG

SEQ ID NO: 2 - protein, 8 aa, synthetic construct
ISAYNGNT

SEQ ID NO: 3 - protein, 17 aa, synthetic construct
ARDWDYDFLTGWDGMDV

SEQ ID NO: 4 - protein, 8 aa, synthetic construct
SSNIGNNY

SEQ ID NO: 5 - skipped

SEQ ID NO: 6 - protein, 12 aa, synthetic construct
GTWDSSLSAGQV

SEQ ID NO: 7 - protein, 8 aa, synthetic construct
GYTFTDYY

SEQ ID NO: 8 - protein, 8 aa, synthetic construct
IDPDNGGT

SEQ ID NO: 9 - protein, 13 aa, synthetic construct
ARALWFGYGFLDY

SEQ ID NO: 10 - protein, 9 aa, synthetic construct
SSDLGGYPF

SEQ ID NO: 11 - skipped

SEQ ID NO: 12 - protein, 10 aa, synthetic construct
TSYTDANTLV

SEQ ID NO: 13 - protein, 8 aa, synthetic construct
GFTFDDYA

SEQ ID NO: 14 - protein, 8 aa, synthetic construct
ISGDGGST

SEQ ID NO: 15 - protein, 21 aa, synthetic construct
AKDMVAAAGWNPYYYYYGMDV

SEQ ID NO: 16 - protein, 8 aa, synthetic construct
SGGIATNY

SEQ ID NO: 17 - skipped

SEQ ID NO: 18 - protein, 10 aa, synthetic construct
QSYDSNNHVV

SEQ ID NO: 19 - protein, 8 aa, synthetic construct
GYTFTGYY

SEQ ID NO: 20 - protein, 8 aa, synthetic construct
INPNSGGT

SEQ ID NO: 21 - protein, 15 aa, synthetic construct
ARHSSRDHYYYGMDV

SEQ ID NO: 22 - protein, 8 aa, synthetic construct
SSNIGNNY

SEQ ID NO: 23 - skipped

SEQ ID NO: 24 - protein, 11 aa, synthetic construct
GTWDSSLNAGV

SEQ ID NO: 25 - protein, 8 aa, synthetic construct
GITVSNNY

SEQ ID NO: 26 - protein, 7 aa, synthetic construct
IYSDGST

SEQ ID NO: 27 - protein, 15 aa, synthetic construct
ASDKVAVAWTDGLDS

SEQ ID NO: 28 - protein, 6 aa, synthetic construct
SLRSYY

SEQ ID NO: 29 - skipped

SEQ ID NO: 30 - protein, 11 aa, synthetic construct
SSRDSSGNHWV

SEQ ID NO: 31 - protein, 8 aa, synthetic construct
GYTFTDYY

SEQ ID NO: 32 - protein, 8 aa, synthetic construct
VDPYDGYT

SEQ ID NO: 33 - protein, 10 aa, synthetic construct
ARFSGTRQDS

SEQ ID NO: 34 - protein, 9 aa, synthetic construct
SSDVGGYNY

SEQ ID NO: 35 - skipped

SEQ ID NO: 36 - protein, 12 aa, synthetic construct
SSYAGSNNLAVV

SEQ ID NO: 37 - protein, 8 aa, synthetic construct
GYTFTDFY

SEQ ID NO: 38 - protein, 8 aa, synthetic construct
VNPKSGGT

SEQ ID NO: 39 - protein, 15 aa, synthetic construct
ARSWQGMSWESVEDV

SEQ ID NO: 40 - protein, 9 aa, synthetic construct
SSNIGAGYD

SEQ ID NO: 41 - skipped

SEQ ID NO: 42 - protein, 11 aa, synthetic construct
QSYDSSLSGAV

SEQ ID NO: 43 - protein, 8 aa, synthetic construct
GYSFTSYW

SEQ ID NO: 44 - protein, 8 aa, synthetic construct
IYPGDSDT

SEQ ID NO: 45 - protein, 10 aa, synthetic construct
ARGGYIYYDA

SEQ ID NO: 46 - protein, 8 aa, synthetic construct
RSNVGNNA

SEQ ID NO: 47 - skipped

SEQ ID NO: 48 - protein, 11 aa, synthetic construct
AAWDDSLNGLV

SEQ ID NO: 49 - protein, 8 aa, synthetic construct
GFTFSSYA

SEQ ID NO: 50 - protein, 8 aa, synthetic construct
ISGSGGST

SEQ ID NO: 51 - protein, 18 aa, synthetic construct
RWGWGPVGRVESHTSGDS

SEQ ID NO: 52 - protein, 9 aa, synthetic construct
SSNIGAGYV

SEQ ID NO: 53 - skipped

SEQ ID NO: 54 - protein, 11 aa, synthetic construct
QSYDSSLSGWV

SEQ ID NO: 55 - protein, 8 aa, synthetic construct
GFTFDDYA

SEQ ID NO: 56 - protein, 8 aa, synthetic construct
ISWNSGSI

SEQ ID NO: 57 - protein, 11 aa, synthetic construct
ARGYMGHNWYD

SEQ ID NO: 58 - protein, 6 aa, synthetic construct
DIGSKA

SEQ ID NO: 59 - skipped

SEQ ID NO: 60 - protein, 11 aa, synthetic construct
QVWDSFSDHYV

SEQ ID NO: 61 - protein, 8 aa, synthetic construct
GFTFSSYW

SEQ ID NO: 62 - protein, 8 aa, synthetic construct
INSDGSST

SEQ ID NO: 63 - protein, 10 aa, synthetic construct
ARSGYYMSDI

SEQ ID NO: 64 - protein, 6 aa, synthetic construct
NIGSKS

SEQ ID NO: 65 - skipped

SEQ ID NO: 66 - protein, 11 aa, synthetic construct
QVWDSSSDQYV

SEQ ID NO: 67 - protein, 8 aa, synthetic construct
GDTFSSYA

SEQ ID NO: 68 - protein, 8 aa, synthetic construct
INLIFGTV

SEQ ID NO: 69 - protein, 17 aa, synthetic construct
ARGHWSQVWWTSHSYDL

SEQ ID NO: 70 - protein, 6 aa, synthetic construct
NIGSKS

SEQ ID NO: 71 - skipped

SEQ ID NO: 72 - protein, 11 aa, synthetic construct
QVWDSSSDHYV

SEQ ID NO: 73 - protein, 8 aa, synthetic construct
GYIFTGYY

SEQ ID NO: 74 - protein, 8 aa, synthetic construct
INPNSGVT

SEQ ID NO: 75 - protein, 11 aa, synthetic construct
AREGVWGYYDS

SEQ ID NO: 76 - protein, 6 aa, synthetic construct
SLRSYY

SEQ ID NO: 77 - skipped

SEQ ID NO: 78 - protein, 12 aa, synthetic construct
NSRDSSGNHHVV

SEQ ID NO: 79 - protein, 8 aa, synthetic construct
GYSFTSYW

SEQ ID NO: 80 - protein, 8 aa, synthetic construct
IDPSDSYT

SEQ ID NO: 81 - protein, 10 aa, synthetic construct
ARMEIYSPDY

SEQ ID NO: 82 - protein, 6 aa, synthetic construct
QSINKY

SEQ ID NO: 83 - skipped

SEQ ID NO: 84 - protein, 9 aa, synthetic construct
QQSYSTPLT

SEQ ID NO: 85 - protein, 124 aa, synthetic construct
EVQLVQSGAEVKKPGASVKVSCKASGYNFTSYGISWVRQAPGQGLEWMGWISAYNGNTNYAQKLQGRVTMTTDTSTSTAYMELRSLRSDDTAVYYCARDWDYDFLTGWDGMDVWGQGTTVTVSS

SEQ ID NO: 86 - protein, 112 aa, synthetic construct
QSVVTQPPSVSAAPGQKVTISCSGSSSNIGNNYVSWYQQLPGTAPKLLIYYNNKRPSGIPDRFSGSKSGTSATLGITGLQTGDEADYYCGTWDSSLSAGQVFGGGTKLTVLG

SEQ ID NO: 87 - protein, 121 aa, synthetic construct
QVQLVQSGAEVKKPGASVKVSCKASGYTFTDYYIHWVRQAPGQGLEWMGWIDPDNGGTNYAQNFQDRVTMTRDTSVSTAYLEVTSLKSDDAAVYYCARALWFGYGFLDYHWGQGTLVTVSS

SEQ ID NO: 88 - protein, 111 aa, synthetic construct
QSALTQPASVSGSPGQSITISCTGSSSDLGGYPFVSWYQQHPDKAPKLIIYDVTNRPSGISNRFSGSKSGYRASLTISGLQAEDEADYYCTSYTDANTLVFGTGTKVTVLG

SEQ ID NO: 89 - protein, 128 aa, synthetic construct
EVQLVESGGGVVQPGGSLRLSCAASGFTFDDYAMHWVRQAPGKGLEWVSLISGDGGSTYYADSVKGRFTISRDNSKNSLYLQMNSLRTEDTALYYCAKDMVAAAGWNPYYYYYGMDVWGQGTTVTVSS

SEQ ID NO: 90 - protein, 112 aa, synthetic construct
NFMLTQPHSVSESPGKTVTISCTRSSGGIATNYVQWYQQRPGNAPTAVIYVDNERPSGVPERFSGSIDTSSNSASLTISGLETEDEADYYCQSYDSNNHVVFGGGTKVTVLG

SEQ ID NO: 91 - protein, 122 aa, synthetic construct
EVQLVQSGAEVKKPGASVKVSCKASGYTFTGYYMHWVRQAPGQGLEWMGWINPNSGGTNYAQKFQGRVTMTRDTSISTAYMELGRLRSDDTAVYYCARHSSRDHYYYGMDVWGQGTTVTVSS

SEQ ID NO: 92 - protein, 112 aa, synthetic construct
QSVVTQPPSVSAAPGQRVTISCSGGSSNIGNNYVSWYQHLPGTAPKLLIYENNLKRPSGIPDRFSGSKSGTSATLGITGLQTGDEADYYCGTWDSSLNAGVFGGGTKLTVLG

SEQ ID NO: 93 - protein, 121 aa, synthetic construct
EVQLVESGGGLVQPGGSLRLSCVVSGITVSNNYMTWVRQAPGKGLEWVSVIYSDGSTYYADSVRGRFTISRDISKNTVFLQMNSLRAEDTAMYYCASDKVAVAWTDGLDSWGQGTMVTVSS

SEQ ID NO: 94 - protein, 109 aa, synthetic construct
SSELTQDPTVSVALGQTVRITCQGDSLRSYYGGWYQQKPGQAPILVFHGQTNRPSGIPDRFSGSSSGNTVSLTITGAQAEDEADYYCSSRDSSGNHWVFGGGTKLTVLG

SEQ ID NO: 95 - protein, 118 aa, synthetic construct
EVQLVQSGAEVKKPGASVKISCKTSGYTFTDYYLHWVRQAPVQGLEWMGYVDPYDGYTHYAQNFQGRVTMTTDTSTSTAYMELSSLRSEDTAVYYCARFSGTRQDSHWGQGTLVTVSS

SEQ ID NO: 96 - protein, 113 aa, synthetic construct
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLAVVFGGGTKLTVLG

SEQ ID NO: 97 - protein, 122 aa, synthetic construct
QVQLVQSGAEVKKPGASVKVSCKASGYTFTDFYIHWVRQAPGQGPEWMGWVNPKSGGTNYAQKFQGRVTMTRDTSISTAYMALSRLRSDDTAVYYCARSWQGMSWESVEDVWGQGTLVTVSS

SEQ ID NO: 98 - protein, 112 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGAVFGTGTKVTVLG

SEQ ID NO: 99 - protein, 117 aa, synthetic construct
EVQLVQSGAEVKKPGESLKISCKGSGYSFTSYWIGWVRQMPGKGLEWMGIIYPGDSDTRYSPSFQGQVTISADKSISTAYLQWSSLKASDTAVYYCARGGYIYYDAWGQGTLVTVSS

SEQ ID NO: 100 - protein, 111 aa, synthetic construct
SYVLTQPPSVSEAPRQRVTISCSGSRSNVGNNAVSWYQHLPGKAPKLLIYYDDLMPSGVSDRFSGSKSGTSASLAISGLQSEDEADYYCAAWDDSLNGLVFGGGTKLTVLG

SEQ ID NO: 101 - protein, 126 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARWGWGPVGRVESHTSGDSWGQGTLVTVSS

SEQ ID NO: 102 - protein, 112 aa, synthetic construct
QSVLTQPPSVSGAPGQRVSISCTGSSSNIGAGYVVHWYQHLPGTVPKLLIYDNSNRPSGVPDRFSGSNSGSSASLAVTGLQAEDEADYYCQSYDSSLSGWVFGGGTKLTVLG

SEQ ID NO: 103 - protein, 118 aa, synthetic construct
QVQLVQSGGGLVQPGRSLRLSCAASGFTFDDYAMHWVRQAPGKGLEWVSGISWNSGSIGYADSVKGRFTISRDNAKNSLYLQMNSLRPEDTAVYYCARGYMGHNWYDWGQGTLVTVSS

SEQ ID NO: 104 - protein, 109 aa, synthetic construct
LPVLTQPPSVSVAPGKTARITCGGNDIGSKAVHWYQQKPGQAPVLVIYYNRDRPSGIPERFSGSNSGNTATLTISRVEAGDEADYYCQVWDSFSDHYVFGTGTKVTVLG

SEQ ID NO: 105 - protein, 117 aa, synthetic construct
QVQLQESGGGLVQPGGSLRLSCAASGFTFSSYWMHWVRQAPGKGLVWVSRINSDGSSTSYADSVKGRFTISRDNAKNTLYLQMNSLRAEDTAVYYCARSGYYMSDIWGQGTLVTVSS

SEQ ID NO: 106 - protein, 109 aa, synthetic construct
NFMLTQPPSLSVAPGKTATITCGGNNIGSKSVHWYQQKPGQAPVLVISYDSDRPSGIPERFSGSKSGNTATLTISRVEAGDEADYYCQVWDSSSDQYVFGSGTKVTVLG

SEQ ID NO: 107 - protein, 124 aa, synthetic construct
QVQLVQSGAEVKKPGSSVKVSCKASGDTFSSYAVSWVRQAPGQGLEWMGAINLIFGTVKYAQKFQGRITITADESTSTAYMELSSLRSEDTAVYYCARGHWSQVWWTSHSYDLWGQGTLVTVSS

SEQ ID NO: 108 - protein, 109 aa, synthetic construct
QAVLTQPPSVSVAPGKTARITCGGNNIGSKSVHWYQQKPGQAPVLVVYDDSDRPSGIPERFSGSNSGNTATLTISRVEAGDEADYYCQVWDSSSDHYVFGTGTKVTVLG

SEQ ID NO: 109 - protein, 118 aa, synthetic construct
QVQLVQSGTEVRKPGASLKVSCTTSGYIFTGYYIHWVRQVPGQGLEWMGWINPNSGVTEFAQGFQGRITMTRDTSTSTVYMELSRLTSDDTAVYYCAREGVWGYYDSWGQGTLVTVSS

SEQ ID NO: 110 - protein, 110 aa, synthetic construct
SSELTQDPAVSVALGQTVRITCQGDSLRSYYASWYQQKPGQAPVLVIYDKNNRPSGIPDRFSGSSSGNTASLTITGAQAEDEADYYCNSRDSSGNHHVVFGGGTKLTVLG

SEQ ID NO: 111 - protein, 117 aa, synthetic construct
EVQLVQSGAEVKKPGESLRISCKDSGYSFTSYWISWVRQMPGKGLEWMGRIDPSDSYTNYSPSFQGHVTISADKSISTAYLQWSSLKASDTAMYYCARMEIYSPDYWGQGTLVTVSS

SEQ ID NO: 112 - protein, 108 aa, synthetic construct
DIQLTQSPSSLSASVGDRVTISCRASQSINKYLNWYQQKPGEAPKLLIYGALRLQSGVPSRFSGSGSGTDYALTITSLQPEDFATYYCQQSYSTPLTFGGGTKVDIKR

SEQ ID NO: 113 - protein, 9 aa, Homo sapiens
RMFPNAPYL

SEQ ID NO: 114 - protein, 9 aa, synthetic construct
RMAAADPYL

SEQ ID NO: 115 - protein, 9 aa, synthetic construct
AMFPNAPYL

SEQ ID NO: 116 - protein, 9 aa, synthetic construct
RMAPNAPYL

SEQ ID NO: 117 - protein, 8 aa, synthetic construct
RMFANAPY

SEQ ID NO: 118 - protein, 9 aa, synthetic construct
RMFPAAPYL

SEQ ID NO: 119 - protein, 9 aa, synthetic construct
RMFPNGPYL

SEQ ID NO: 120 - protein, 9 aa, synthetic construct
RMFPNAAYL

SEQ ID NO: 121 - protein, 9 aa, synthetic construct
RMFPNAPAL

SEQ ID NO: 122 - protein, 9 aa, Alphapapillomavirus sp.
YMLDLQPET

SEQ ID NO: 123 - protein, 126 aa, synthetic construct
EVQLLESGGGLVQPGGSLRLSCAASGFTFSSYAMSWVRQAPGKGLEWVSAISGSGGSTYYGDSVKGRFTISRDNSKNTLYLQMNSLRAEDTAVYYCARWGWGPVGRVESHTSGDSWGQGTLVTVSS

SEQ ID NO: 124 - protein, 126 aa, synthetic construct
EVQLVESGGGLVQPGGSLRLSCAASGFTFSSYAMSWVRQAPGKGLEWVSAISGSGGSTYYGDSVKGRFTISRDNSKNTLYLQMNSLRAEDTAVYYCARWGWGPVGRVESHTSGDSWGQGTLVTVSS

SEQ ID NO: 125 - protein, 126 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARWGWGPVGRVESHTSGDSWGQGTLVTVSS

SEQ ID NO: 126 - protein, 111 aa, synthetic construct
QSVLTQPPSVSGAPGQRVTISCTGSSSNIGAGYVVHWYQQLPGTAPKLLIYDNSNRPSGVPDRFSGSKSGTSASLAVTGLQAEDEADYYCQSYDSSLSGWVFGGGTKLTVL

SEQ ID NO: 127 - protein, 111 aa, synthetic construct
QSVLTQPPSVSGAPGQRVTISCTGSSSNIGAGYVVHWYQHLPGTAPKLLIYDNSNRPSGVPDRFSGSNSGSSASLAVTGLQAEDEADYYCQSYDSSLSGWVFGGGTKLTVL

SEQ ID NO: 128 - protein, 111 aa, synthetic construct
QSVLTQPPSVSGAPGQRVSISCTGSSSNIGAGYVVHWYQQLPGTVPKLLIYDNSNRPSGVPDRFSGSNSGTSASLAVTGLQAEDEADYYCQSYDSSLSGWVFGGGTKLTVL

SEQ ID NO: 129 - protein, 111 aa, synthetic construct
QSVLTQPPSVSGAPGQRVTISCTGSSSNIGAGYVVHWYQHLPGTVPKLLIYDNSNRPSGVPDRFSGSKSGTSASLAVTGLQAEDEADYYCQSYDSSLSGWVFGGGTKLTVL

SEQ ID NO: 130 - protein, 118 aa, synthetic construct
QVQLVQSGAEVKKPGASLKVSCTTSGYIFTGYYIHWVRQAPGQGLEWMGWINPNSGVTEFAQGFQGRITMTRDTSTSTAYMELSRLTSDDTAVYYCAREGVWGYYDSWGQGTLVTVSS

SEQ ID NO: 131 - protein, 118 aa, synthetic construct
QVQLVQSGTEVKKPGASLKVSCTTSGYIFTGYYIHWVRQVPGQGLEWMGWINPNSGVTEFAQGFQGRITMTRDTSTSTAYMELSRLTSDDTAVYYCAREGVWGYYDSWGQGTLVTVSS

SEQ ID NO: 132 - protein, 118 aa, synthetic construct
QVQLVQSGAEVRKPGASLKVSCTTSGYIFTGYYIHWVRQAPGQGLEWMGWINPNSGVTEFAQGFQGRITMTRDTSTSTVYMELSRLTSDDTAVYYCAREGVWGYYDSWGQGTLVTVSS

SEQ ID NO: 133 - protein, 118 aa, synthetic construct
QVQLVQSGTEVKKPGASLKVSCTTSGYIFTGYYIHWVRQAPGQGLEWMGWINPNSGVTEFAQGFQGRITMTRDTSTSTAYMELSRLTSDDTAVYYCAREGVWGYYDSWGQGTLVTVSS

SEQ ID NO: 134 - protein, 109 aa, synthetic construct
SYELTQDPAVSVALGQTVRITCQGDSLRSYYASWYQQKPGQAPVRVIYDKNNRPSGIPDRFSGSSSGNTASLTITGAQAEDEADYYCNSRDSSGNHHVVFGGGTKLTVL

SEQ ID NO: 135 - protein, 109 aa, synthetic construct
SYELTQDPAVSVALGQTVRITCQGDSLRSYYASWYQQKPGQAPVLVIYDKNNRPSGIPDRFSGSSSGNTASLTITGAQAEDEADYYCNSRDSSGNHHVVFGGGTKLTVL

SEQ ID NO: 136 - protein, 109 aa, synthetic construct
SSELTQDPAVSVALGQTVRITCQGDSLRSYYASWYQQKPGQAPVRVIYDKNNRPSGIPDRFSGSSSGNTASLTITGAQAEDEADYYCNSRDSSGNHHVVFGGGTKLTVL

SEQ ID NO: 137 - protein, 25 aa, Homo sapiens
EVQLVQSGAEVKKPGASVKVSCKAS

SEQ ID NO: 138 - protein, 17 aa, Homo sapiens
ISWVRQAPGQGLEWMGW

SEQ ID NO: 139 - protein, 38 aa, Homo sapiens [sequence omitted]

SEQ ID NO: 140 - protein, 11 aa, Homo sapiens
WGQGTTVTVSS

SEQ ID NO: 141 - protein, 25 aa, Homo sapiens
QSVVTQPPSVSAAPGQKVTISCSGS

SEQ ID NO: 142 - protein, 17 aa, Homo sapiens
VSWYQQLPGTAPKLLIY

SEQ ID NO: 143 - protein, 36 aa, Homo sapiens
KRPSGIPDRFSGSKSGTSATLGITGLQTGDEADYYC

SEQ ID NO: 144 - protein, 11 aa, Homo sapiens
FGGGTKLTVLG

SEQ ID NO: 145 - protein, 25 aa, Homo sapiens [sequence omitted]

SEQ ID NO: 146 - protein, 17 aa, Homo sapiens
IHWVRQAPGQGLEWMGW

SEQ ID NO: 147 - protein, 38 aa, Homo sapiens
NYAQNFQDRVTMTRDTSVSTAYLEVTSLKSDDAAVYYC

SEQ ID NO: 148 - protein, 12 aa, Homo sapiens
HWGQGTLVTVSS

SEQ ID NO: 149 - protein, 25 aa, Homo sapiens
QSALTQPASVSGSPGQSITISCTGS

SEQ ID NO: 150 - protein, 17 aa, Homo sapiens
VSWYQQHPDKAPKLIIY

SEQ ID NO: 151 - protein, 36 aa, Homo sapiens
NRPSGISNRFSGSKSGYRASLTISGLQAEDEADYYC

SEQ ID NO: 152 - protein, 11 aa, Homo sapiens
FGTGTKVTVLG

SEQ ID NO: 153 - protein, 25 aa, Homo sapiens
EVQLVESGGGVVQPGGSLRLSCAAS

SEQ ID NO: 154 - protein, 17 aa, Homo sapiens
MHWVRQAPGKGLEWVSL

SEQ ID NO: 155 - protein, 38 aa, Homo sapiens
YYADSVKGRFTISRDNSKNSLYLQMNSLRTEDTALYYC

SEQ ID NO: 156 - protein, 11 aa, Homo sapiens
WGQGTTVTVSS

SEQ ID NO: 157 - protein, 25 aa, Homo sapiens
NFMLTQPHSVSESPGKTVTISCTRS

SEQ ID NO: 158 - protein, 17 aa, Homo sapiens
VQWYQQRPGNAPTAVIY

SEQ ID NO: 159 - protein, 38 aa, Homo sapiens
ERPSGVPERFSGSIDTSSNSASLTISGLETEDEADYYC

SEQ ID NO: 160 - protein, 11 aa, Homo sapiens
FGGGTKVTVLG

SEQ ID NO: 161 - protein, 25 aa, Homo sapiens
EVQLVQSGAEVKKPGASVKVSCKAS

SEQ ID NO: 162 - protein, 17 aa, Homo sapiens
MHWVRQAPGQGLEWMGW

SEQ ID NO: 163 - protein, 38 aa, Homo sapiens
NYAQKFQGRVTMTRDTSISTAYMELGRLRSDDTAVYYC

SEQ ID NO: 164 - protein, 11 aa, Homo sapiens
WGQGTTVTVSS

SEQ ID NO: 165 - protein, 25 aa, Homo sapiens
QSVVTQPPSVSAAPGQRVTISCSGG

SEQ ID NO: 166 - protein, 17 aa, Homo sapiens
VSWYQHLPGTAPKLLIY

SEQ ID NO: 167 - protein, 37 aa, Homo sapiens
LKRPSGIPDRFSGSKSGTSATLGITGLQTGDEADYYC

SEQ ID NO: 168 - protein, 11 aa, Homo sapiens
FGGGTKLTVLG

SEQ ID NO: 169 - protein, 25 aa, Homo sapiens
EVQLVESGGGLVQPGGSLRLSCVVS

SEQ ID NO: 170 - protein, 17 aa, Homo sapiens
MTWVRQAPGKGLEWVSV

SEQ ID NO: 171 - protein, 38 aa, Homo sapiens
YYADSVRGRFTISRDISKNTVFLQMNSLRAEDTAMYYC

SEQ ID NO: 172 - protein, 11 aa, Homo sapiens
WGQGTMVTVSS

SEQ ID NO: 173 - protein, 25 aa, Homo sapiens
SSELTQDPTVSVALGQTVRITCQGD

SEQ ID NO: 174 - protein, 17 aa, Homo sapiens
GGWYQQKPGQAPILVFH

SEQ ID NO: 175 - protein, 36 aa, Homo sapiens
NRPSGIPDRFSGSSSGNTVSLTITGAQAEDEADYYC

SEQ ID NO: 176 - protein, 11 aa, Homo sapiens
FGGGTKLTVLG

SEQ ID NO: 177 - protein, 25 aa, Homo sapiens
EVQLVQSGAEVKKPGASVKISCKTS

SEQ ID NO: 178 - protein, 17 aa, Homo sapiens
LHWVRQAPVQGLEWMGY

SEQ ID NO: 179 - protein, 38 aa, Homo sapiens
HYAQNFQGRVTMTTDTSTSTAYMELSSLRSEDTAVYYC

SEQ ID NO: 180 - protein, 12 aa, Homo sapiens
HWGQGTLVTVSS

SEQ ID NO: 181 - protein, 25 aa, Homo sapiens
QSALTQPPSASGSPGQSVTISCTGT

SEQ ID NO: 182 - protein, 17 aa, Homo sapiens
VSWYQQHPGKAPKLMIY

SEQ ID NO: 183 - protein, 36 aa, Homo sapiens
KRPSGVPDRFSGSKSGNTASLTVSGLQAEDEADYYC

SEQ ID NO: 184 - protein, 11 aa, Homo sapiens
FGGGTKLTVLG

SEQ ID NO: 185 - protein, 25 aa, Homo sapiens [sequence omitted]

SEQ ID NO: 186 - protein, 17 aa, Homo sapiens
IHWVRQAPGQGPEWMGW

SEQ ID NO: 187 - protein, 38 aa, Homo sapiens
NYAQKFQGRVTMTRDTSISTAYMALSRLRSDDTAVYYC

SEQ ID NO: 188 - protein, 11 aa, Homo sapiens
WGQGTLVTVSS

SEQ ID NO: 189 - protein, 25 aa, Homo sapiens
QSVLTQPPSVSGAPGQRVTISCTGS

SEQ ID NO: 190 - protein, 17 aa, Homo sapiens
VHWYQQLPGTAPKLLIY

SEQ ID NO: 191 - protein, 36 aa, Homo sapiens
NRPSGVPDRFSGSKSGTSASLAITGLQAEDEADYYC

SEQ ID NO: 192 - protein, 11 aa, Homo sapiens
FGTGTKVTVLG

SEQ ID NO: 193 - protein, 25 aa, Homo sapiens
EVQLVQSGAEVKKPGESLKISCKGS

SEQ ID NO: 194 - protein, 17 aa, Homo sapiens
IGWVRQMPGKGLEWMGI

SEQ ID NO: 195 - protein, 38 aa, Homo sapiens
RYSPSFQGQVTISADKSISTAYLQWSSLKASDTAVYYC

SEQ ID NO: 196 - protein, 11 aa, Homo sapiens
WGQGTLVTVSS

SEQ ID NO: 197 - protein, 25 aa, Homo sapiens
SYVLTQPPSVSEAPRQRVTISCSGS

SEQ ID NO: 198 - protein, 17 aa, Homo sapiens
VSWYQHLPGKAPKLLIY

SEQ ID NO: 199 - protein, 36 aa, Homo sapiens
LMPSGVSDRFSGSKSGTSASLAISGLQSEDEADYYC

SEQ ID NO: 200 - protein, 11 aa, Homo sapiens
FGGGTKLTVLG

SEQ ID NO: 201 - protein, 25 aa, Homo sapiens [sequence omitted]

SEQ ID NO: 202 - protein, 17 aa, Homo sapiens
MSWVRQAPGKGLEWVSA

SEQ ID NO: 203 - protein, 39 aa, Homo sapiens
YYADSVKGRFTISRDNSKNTLYLQMNSLRAEDTAVYYCA

SEQ ID NO: 204 - protein, 11 aa, Homo sapiens
WGQGTLVTVSS

SEQ ID NO: 205 - protein, 25 aa, Homo sapiens
QSVLTQPPSVSGAPGQRVSISCTGS

SEQ ID NO: 206 - protein, 17 aa, Homo sapiens
VHWYQHLPGTVPKLLIY

SEQ ID NO: 207 - protein, 36 aa, Homo sapiens
NRPSGVPDRFSGSNSGSSASLAVTGLQAEDEADYYC

SEQ ID NO: 208 - protein, 11 aa, Homo sapiens
FGGGTKLTVLG

SEQ ID NO: 209 - protein, 25 aa, Homo sapiens
QVQLVQSGGGLVQPGRSLRLSCAAS

SEQ ID NO: 210 - protein, 17 aa, Homo sapiens
MHWVRQAPGKGLEWVSG

SEQ ID NO: 211 - protein, 38 aa, Homo sapiens
GYADSVKGRFTISRDNAKNSLYLQMNSLRPEDTAVYYC

SEQ ID NO: 212 - protein, 11 aa, Homo sapiens
WGQGTLVTVSS

SEQ ID NO: 213 - protein, 25 aa, Homo sapiens
LPVLTQPPSVSVAPGKTARITCGGN

SEQ ID NO: 214 - protein, 17 aa, Homo sapiens
VHWYQQKPGQAPVLVIY

SEQ ID NO: 215 - protein, 36 aa, Homo sapiens
DRPSGIPERFSGSNSGNTATLTISRVEAGDEADYYC

SEQ ID NO: 216 - protein, 11 aa, Homo sapiens
FGTGTKVTVLG

SEQ ID NO: 217 - protein, 25 aa, Homo sapiens
QVQLQESGGGLVQPGGSLRLSCAAS

SEQ ID NO: 218 - protein, 17 aa, Homo sapiens
MHWVRQAPGKGLVWVSR

SEQ ID NO: 219 - protein, 38 aa, Homo sapiens
SYADSVKGRFTISRDNAKNTLYLQMNSLRAEDTAVYYC

SEQ ID NO: 220 - protein, 11 aa, Homo sapiens
WGQGTLVTVSS

SEQ ID NO: 221 - protein, 25 aa, Homo sapiens
NFMLTQPPSLSVAPGKTATITCGGN

SEQ ID NO: 222 - protein, 17 aa, Homo sapiens
VHWYQQKPGQAPVLVIS

SEQ ID NO: 223 - protein, 36 aa, Homo sapiens
DRPSGIPERFSGSKSGNTATLTISRVEAGDEADYYC

SEQ ID NO: 224 - protein, 11 aa, Homo sapiens
FGSGTKVTVLG

SEQ ID NO: 225 - protein, 25 aa, Homo sapiens [sequence omitted]

SEQ ID NO: 226 - protein, 17 aa, Homo sapiens
VSWVRQAPGQGLEWMGA

SEQ ID NO: 227 - protein, 38 aa, Homo sapiens
KYAQKFQGRITITADESTSTAYMELSSLRSEDTAVYYC

SEQ ID NO: 228 - protein, 11 aa, Homo sapiens
WGQGTLVTVSS

SEQ ID NO: 229 - protein, 25 aa, Homo sapiens
QAVLTQPPSVSVAPGKTARITCGGN

SEQ ID NO: 230 - protein, 17 aa, Homo sapiens
VHWYQQKPGQAPVLVVY

SEQ ID NO: 231 - protein, 36 aa, Homo sapiens; identical to SEQ ID NO: 215

SEQ ID NO: 232 - protein, 11 aa, Homo sapiens
FGTGTKVTVLG

SEQ ID NO: 233 - protein, 25 aa, Homo sapiens
QVQLVQSGTEVRKPGASLKVSCTTS

SEQ ID NO: 234 - protein, 17 aa, Homo sapiens
IHWVRQVPGQGLEWMGW

SEQ ID NO: 235 - protein, 38 aa, Homo sapiens
EFAQGFQGRITMTRDTSTSTVYMELSRLTSDDTAVYYC

SEQ ID NO: 236 - protein, 11 aa, Homo sapiens
WGQGTLVTVSS

SEQ ID NO: 237 - protein, 25 aa, Homo sapiens
SSELTQDPAVSVALGQTVRITCQGD

SEQ ID NO: 238 - protein, 17 aa, Homo sapiens
ASWYQQKPGQAPVLVIY

SEQ ID NO: 239 - protein, 36 aa, Homo sapiens
NRPSGIPDRFSGSSSGNTASLTITGAQAEDEADYYC

SEQ ID NO: 240 - protein, 11 aa, Homo sapiens
FGGGTKLTVLG

SEQ ID NO: 241 - protein, 25 aa, Homo sapiens
EVQLVQSGAEVKKPGESLRISCKDS

SEQ ID NO: 242 - protein, 17 aa, Homo sapiens
ISWVRQMPGKGLEWMGR

SEQ ID NO: 243 - protein, 38 aa, Homo sapiens
NYSPSFQGHVTISADKSISTAYLQWSSLKASDTAMYYC

SEQ ID NO: 244 - protein, 11 aa, Homo sapiens
WGQGTLVTVSS

SEQ ID NO: 245 - protein, 26 aa, Homo sapiens
DIQLTQSPSSLSASVGDRVTISCRAS

SEQ ID NO: 246 - protein, 17 aa, Homo sapiens
LNWYQQKPGEAPKLLIY

SEQ ID NO: 247 - protein, 36 aa, Homo sapiens
RLQSGVPSRFSGSGSGTDYALTITSLQPEDFATYYC

SEQ ID NO: 248 - protein, 11 aa, Homo sapiens
FGGGTKVDIKR

SEQ ID NO: 249 - protein, 21 aa, synthetic construct
SRGGGGSGGGGSGGGGSLEMA

SEQ ID NO: 250 - protein, 5 aa, synthetic construct
GGGGS

SEQ ID NO: 251 - protein, 4 aa, synthetic construct
SGGG

SEQ ID NO: 252 - protein, 12 aa, synthetic construct
GGSGGSGGSGGS

SEQ ID NO: 253 - protein, 5 aa, synthetic construct
RAKRS

SEQ ID NO: 254 - protein, 10 aa, synthetic construct
GGGGSGGGGS

SEQ ID NO: 255 - protein, 4 aa, synthetic construct
GSGS

SEQ ID NO: 256 - protein, 4 aa, synthetic construct
GGSG

SEQ ID NO: 257 - protein, 27 aa, synthetic construct
GSGAPVKQTLNFDLLKLAGDVESNPGP

SEQ ID NO: 258 - protein, 15 aa, synthetic construct
GGGGSGGGGSGGGGS

SEQ ID NO: 259 - protein, 6 aa, synthetic construct
GSGSGS

SEQ ID NO: 260 - protein, 8 aa, synthetic construct
GGSGGGSG

SEQ ID NO: 261 - protein, 32 aa, synthetic construct
RAKRSGSGAPVKQTLNFDLLKLAGDVESNPGP

SEQ ID NO: 262 - protein, 5 aa, synthetic construct
AAATG

SEQ ID NO: 263 - protein, 8 aa, synthetic construct
GSGSGSGS

SEQ ID NO: 264 - protein, 12 aa, synthetic construct
GGSGGGSGGGSG

SEQ ID NO: 265 - protein, 22 aa, synthetic construct
GSGATNFSLLKQAGDVEENPGP

SEQ ID NO: 266 - protein, 11 aa, synthetic construct
TPLGDTTHTSG

SEQ ID NO: 267 - protein, 10 aa, synthetic construct
GSGSGSGSGS

SEQ ID NO: 268 - protein, 27 aa, synthetic construct
RAKRSGSGATNFSLLKQAGDVEENPGP

SEQ ID NO: 269 - skipped

SEQ ID NO: 270 - protein, 6 aa, synthetic construct
GGSGGS

SEQ ID NO: 271 - protein, 22 aa, synthetic construct
GSRGGGGSGGGGSGGGGSLEMA

SEQ ID NO: 272 - protein, 4 aa, synthetic construct
GGSG

SEQ ID NO: 273 - protein, 9 aa, synthetic construct
GGSGGSGGS

SEQ ID NO: 274 - protein, 10 aa, synthetic construct
EQKLISEEDL

SEQ ID NO: 275 - protein, 22 aa, synthetic construct
DYKDHDGDYKDHDIDYKDDDDK

SEQ ID NO: 276 - protein, 8 aa, synthetic construct
DYKDDDDK

SEQ ID NO: 277 - protein, 6 aa, synthetic construct
HHHHHH

SEQ ID NO: 278 - protein, 9 aa, synthetic construct
YPYDVPDYA

SEQ ID NO: 279 - protein, 10 aa, synthetic construct
YPYDVPDYAS

SEQ ID NO: 280 - protein, 20 aa, synthetic construct
METDTLLLWVLLLWVPGSTG

SEQ ID NO: 281 - protein, 42 aa, synthetic construct
KRGRKKLLYIFKQPFMRPVQTTQEEDGCSCRFPEEEEGGCEL

SEQ ID NO: 282 - protein, 48 aa, synthetic construct
QRRKYRSNKGESPVEPAEPCRYSCPREEEGSTIPIQEDYRKPEPACSP

SEQ ID NO: 283 - protein, 41 aa, synthetic construct
RSKRSRLLHSDYMNMTPRRPGPTRKHYQPYAPPRDFAAYRS

SEQ ID NO: 284 - protein, 188 aa, synthetic construct
HRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 285 - protein, 42 aa, synthetic construct
ALYLLRRDQRLPPDAHKPPGGGSFRTPIQEEQADAHSTLAKI

SEQ ID NO: 286 - protein, 38 aa, synthetic construct
CWLTKKKYSSSVHDPNGEYMFMRAVNTAKKSRLTDVTL

SEQ ID NO: 287 - protein, 23 aa, synthetic construct
CARPRRSPAQEDGKVYINMPGRG

SEQ ID NO: 288 - protein, 62 aa, synthetic construct
KKVAKKPTNKAPHPKQEPQEINFPDDLPGSNTAAPVQETLHGCQPVTQEDGKESRISVQERQ

SEQ ID NO: 289 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 290 - protein, 107 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 291 - protein, 120 aa, synthetic construct
EQKLISEEDLAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 292 - protein, 109 aa, synthetic construct
EQKLISEEDLAAATGPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 293 - protein, 99 aa, synthetic construct
AAATGPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 294 - protein, 134 aa, synthetic construct
EQKLISEEDLAAATGPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP

SEQ ID NO: 295 - protein, 124 aa, synthetic construct
AAATGPTHLPYVSEMLEARTAGHMQTLADFRQLPARTLSTHWPPQRSLCSSDFIRILVIFSGMFLVFTLAGALFLHQRRKYRSNKGESPVEPAEPCRYSCPREEEGSTIPIQEDYRKPEPACSP

SEQ ID NO: 296 - protein, 285 aa, synthetic construct
EQKLISEEDLAAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 297 - protein, 275 aa, synthetic construct
AAATGAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 298 - protein, 125 aa, synthetic construct
EQKLISEEDLAAATGDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 299 - protein, 115 aa, synthetic construct
AAATGDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 300 - protein, 132 aa, synthetic construct
EQKLISEEDLAAATGTTTPAPRPPTPAPTIASQPLSLRPEACRPAAGGAVHTRGLDFACDIYIWAPLAGTGCVLLLSLVITLYCQRRKYRSNKGESPVEPAEPCRYSCPREEEGSTIPIQEDYRKPEPACSP

SEQ ID NO: 301 - protein, 122 aa, synthetic construct
AAATGTTTPAPRPPTPAPTIASQPLSLRPEACRPAAGGAVHTRGLDFACDIYIWAPLAGTGCVLLLSLVITLYCQRRKYRSNKGESPVEPAEPCRYSCPREEEGSTIPIQEDYRKPEPACSP

SEQ ID NO: 302 - protein, 272 aa, synthetic construct
EQKLISEEDLAAATGTTTPAPRPPTPAPTIASQPLSLRPEACRPAAGGAVHTRGLDFACDIYIWAPLAGTGCVLLLSLVITLYCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 303 - protein, 262 aa, synthetic construct
AAATGTTTPAPRPPTPAPTIASQPLSLRPEACRPAAGGAVHTRGLDFACDIYIWAPLAGTGCVLLLSLVITLYCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 304 - protein, 126 aa, synthetic construct
EQKLISEEDLAAATGTTTPAPRPPTPAPTIASQPLSLRPEACRPAAGGAVHTRGLDFACDIYIWAPLAGTGCVLLLSLVITLYCALYLLRRDQRLPPDAHKPPGGGSFRTPIQEEQADAHSTLAKI

SEQ ID NO: 305 - protein, 116 aa, synthetic construct
AAATGTTTPAPRPPTPAPTIASQPLSLRPEACRPAAGGAVHTRGLDFACDIYIWAPLAGTGCVLLLSLVITLYCALYLLRRDQRLPPDAHKPPGGGSFRTPIQEEQADAHSTLAKI

SEQ ID NO: 306 - protein, 126 aa, synthetic construct
EQKLISEEDLAAATGTTTPAPRPPTPAPTIASQPLSLRPEACRPAAGGAVHTRGLDFACDIYIWAPLAGTGCVLLLSLVITLYCKRGRKKLLYIFKQPFMRPVQTTQEEDGCSCRFPEEEEGGCEL

SEQ ID NO: 307 - protein, 116 aa, synthetic construct
AAATGTTTPAPRPPTPAPTIASQPLSLRPEACRPAAGGAVHTRGLDFACDIYIWAPLAGTGCVLLLSLVITLYCKRGRKKLLYIFKQPFMRPVQTTQEEDGCSCRFPEEEEGGCEL

SEQ ID NO: 308 - protein, 21 aa, synthetic construct
IYIWAPLAGTCGVLLLSLVIT

SEQ ID NO: 309 - protein, 27 aa, synthetic construct
IISFFLALTSTALLFLLFFLTLRFSVV

SEQ ID NO: 310 - protein, 21 aa, synthetic construct
ILVIFSGMFLVFTLAGALFLH

SEQ ID NO: 311 - protein, 27 aa, synthetic construct
FWVLVVVGGVLACYSLLVTVAFIIFWV

SEQ ID NO: 312 - protein, 28 aa, synthetic construct
PVLDAGPVLFWVILVLVVVVGSSAFLLC

SEQ ID NO: 313 - protein, 21 aa, synthetic construct
VAAILGLGLVLGLLGPLAILL

SEQ ID NO: 314 - protein, 20 aa, synthetic construct
FWLPIGCAAFVVCILGCILI

SEQ ID NO: 315 - protein, 22 aa, synthetic construct
LLAGLVAADAVASLLIVGAVFL

SEQ ID NO: 316 - protein, 22 aa, synthetic construct
ALVVIPIIFGILFAILLVLVFI

SEQ ID NO: 317 - protein, 115 aa, synthetic construct
EVQLQQSGPELVKPGASVKISCKASGYTFTDYNMHWVKQSHGKSLEWIGYIYPYNGGTGYNQKFKSKATLTVDNSSSTAYMDVRSLTSEDSAVYYCARGRPAMDYWGQGTSVTVS

SEQ ID NO: 318 - protein, 111 aa, synthetic construct
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIK

SEQ ID NO: 319 - protein, 6 aa, synthetic construct
TDYNMH

SEQ ID NO: 320 - protein, 17 aa, synthetic construct
YIYPYNGGTGYNQKFKS

SEQ ID NO: 321 - protein, 8 aa, synthetic construct
RGRPAMDY

SEQ ID NO: 322 - protein, 15 aa, synthetic construct
RASESVDNYGISFMN

SEQ ID NO: 323 - protein, 7 aa, synthetic construct
AASNQGS

SEQ ID NO: 324 - protein, 9 aa, synthetic construct
QQSKEVPWT

SEQ ID NO: 325 - protein, 60 aa, unidentified
IPEDTFFPSPESSCDVKLVEKSFETDTNLNFQNLSVIGFRILLLKVAGFNLLMTLRLWSS

SEQ ID NO: 326 - protein, 51 aa, unidentified
GRADCGFTSVSYQQGVLSATILYEILLGKATLYAVLVSALVLMAMVKRKDF

SEQ ID NO: 327 - protein, 68 aa, unidentified
EVKTDSTDHVKPKETENTKQPSKSCHKPKAIVHTEKVNMMSLTVLGLRMLFAKTVAVNFLLTAKLFFL

SEQ ID NO: 328 - protein, 67 aa, unidentified
PIKTDVITMDPKDNCSKDANDTLLLQLTNTSAYYMYLLLLLKSVVYFAIITCCLLRRTAFCCNGEKS

SEQ ID NO: 329 - protein, 21 aa, synthetic construct
GSGEGRGSLLTCGDVEENPGP

SEQ ID NO: 330 - protein, 9 aa, Homo sapiens
SLYPSAPFL

SEQ ID NO: 331 - protein, 10 aa, Homo sapiens
SLYPSAPFLA

SEQ ID NO: 332 - protein, 10 aa, Homo sapiens
EMFPQVPYHL

SEQ ID NO: 333 - protein, 10 aa, Homo sapiens
RLFPNAKFLL

SEQ ID NO: 334 - protein, 9 aa, synthetic construct
  VARIANT 1: note = Any amino acid
  VARIANT 2: note = L, M, or V
  VARIANT 9: note = L, V, or I
XXFPNAPYX

SEQ ID NO: 335 - protein, 257 aa, synthetic construct
QSVVTQPPSVSAAPGQKVTISCSGSSSNIGNNYVSWYQQLPGTAPKLLIYYNNKRPSGIPDRFSGSKSGTSATLGITGLQTGDEADYYCGTWDSSLSAGQVFGGGTKLTVLGSRGGGGSGGGGSGGGGSLEMAEVQLVQSGAEVKKPGASVKVSCKASGYNFTSYGISWVRQAPGQGLEWMGWISAYNGNTNYAQKLQGRVTMTTDTSTSTAYMELRSLRSDDTAVYYCARDWDYDFLTGWDGMDVWGQGTTVTVSS

SEQ ID NO: 336 - protein, 252 aa, synthetic construct
QSALTQPASVSGSPGQSITISCTGSSSDLGGYPFVSWYQQHPDKAPKLIIYDVTNRPSGISNRFSGSKSGYRASLTISGLQAEDEADYYCTSYTDANTLVFGTGTKVTVLGSRGGGGSGGGGSGGGGSLEMAQVQLVQSGAEVKKPGASVKVSCKASGYTFTDYYIHWVRQAPGQGLEWMGWIDPDNGGTNYAQNFQDRVTMTRDTSVSTAYLEVTSLKSDDAAVYYCARALWFGYGFLDYWGQGTLVTVSS

SEQ ID NO: 337 - protein, 261 aa, synthetic construct
NFMLTQPHSVSESPGKTVTISCTRSSGGIATNYVQWYQQRPGNAPTAVIYVDNERPSGVPERFSGSIDTSSNSASLTISGLETEDEADYYCQSYDSNNHVVFGGGTKVTVLGSRGGGGSGGGGSGGGGSLEMAEVQLVESGGGVVQPGGSLRLSCAASGFTFDDYAMHWVRQAPGKGLEWVSLISGDGGSTYYADSVKGRFTISRDNSKNSLYLQMNSLRTEDTALYYCAKDMVAAAGWNPYYYYYGMDVWGQGTTVTVSS

SEQ ID NO: 338 - protein, 254 aa, synthetic construct
QSVVTQPPSVSAAPGQRVTISCSGGSSNIGNNYVSWYQHLPGTAPKLLIYENNKRPSGIPDRFSGSKSGTSATLGITGLQTGDEADYYCGTWDSSLNAGVFGGGTKLTVLGSRGGGGSGGGGSGGGGSLEMAEVQLVQSGAEVKKPGASVKVSCKASGYTFTGYYMHWVRQAPGQGLEWMGWINPNSGGTNYAQKFQGRVTMTRDTSISTAYMELGRLRSDDTAVYYCARHSSRDHYYYGMDVWGQGTTVTVSS

SEQ ID NO: 339 - protein, 251 aa, synthetic construct
SSELTQDPTVSVALGQTVRITCQGDSLRSYYGGWYQQKPGQAPILVFHGQTNRPSGIPDRFSGSSSGNTVSLTITGAQAEDEADYYCSSRDSSGNHWVFGGGTKLTVLGSRGGGGSGGGGSGGGGSLEMAEVQLVESGGGLVQPGGSLRLSCVVSGITVSNNYMTWVRQAPGKGLEWVSVIYSDGSTYYADSVRGRFTISRDISKNTVFLQMNSLRAEDTAMYYCASDKVAVAWTDGLDSWGQGTMVTVSS

SEQ ID NO: 340 - protein, 251 aa, synthetic construct
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLAVVFGGGTKLTVLGSRGGGGSGGGGSGGGGSLEMAEVQLVQSGAEVKKPGASVKISCKTSGYTFTDYYLHWVRQAPVQGLEWMGYVDPYDGYTHYAQNFQGRVTMTTDTSTSTAYMELSSLRSEDTAVYYCARFSGTRQDSWGQGTLVTVSS

SEQ ID NO: 341 - protein, 255 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGAVFGTGTKVTVLGSRGGGGSGGGGSGGGGSLEMAQVQLVQSGAEVKKPGASVKVSCKASGYTFTDFYIHWVRQAPGQGPEWMGWVNPKSGGTNYAQKFQGRVTMTRDTSISTAYMALSRLRSDDTAVYYCARSWQGMSWESVEDVWGQGTLVTVSS

SEQ ID NO: 342 - protein, 249 aa, synthetic construct
SYVLTQPPSVSEAPRQRVTISCSGSRSNVGNNAVSWYQHLPGKAPKLLIYYDDLMPSGVSDRFSGSKSGTSASLAISGLQSEDEADYYCAAWDDSLNGLVFGGGTKLTVLGSRGGGGSGGGGSGGGGSLEMAEVQLVQSGAEVKKPGESLKISCKGSGYSFTSYWIGWVRQMPGKGLEWMGIIYPGDSDTRYSPSFQGQVTISADKSISTAYLQWSSLKASDTAVYYCARGGYIYYDAWGQGTLVTVSS

SEQ ID NO: 343 - protein, 259 aa, synthetic construct
QSVLTQPPSVSGAPGQRVSISCTGSSSNIGAGYVVHWYQHLPGTVPKLLIYDNSNRPSGVPDRFSGSNSGSSASLAVTGLQAEDEADYYCQSYDSSLSGWVFGGGTKLTVLGSRGGGGSGGGGSGGGGSLEMAEVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARWGWGPVGRVESHTSGDSWGQGTLVTVSS

SEQ ID NO: 344 - protein, 248 aa, synthetic construct
LPVLTQPPSVSVAPGKTARITCGGNDIGSKAVHWYQQKPGQAPVLVIYYNRDRPSGIPERFSGSNSGNTATLTISRVEAGDEADYYCQVWDSFSDHYVFGTGTKVTVLGSRGGGGSGGGGSGGGGSLEMAQVQLVQSGGGLVQPGRSLRLSCAASGFTFDDYAMHWVRQAPGKGLEWVSGISWNSGSIGYADSVKGRFTISRDNAKNSLYLQMNSLRPEDTAVYYCARGYMGHNWYDWGQGTLVTVSS

SEQ ID NO: 345 - protein, 247 aa, synthetic construct
NFMLTQPPSLSVAPGKTATITCGGNNIGSKSVHWYQQKPGQAPVLVISYDSDRPSGIPERFSGSKSGNTATLTISRVEAGDEADYYCQVWDSSSDQYVFGSGTKVTVLGSRGGGGSGGGGSGGGGSLEMAQVQLQESGGGLVQPGGSLRLSCAASGFTFSSYWMHWVRQAPGKGLVWVSRINSDGSSTSYADSVKGRFTISRDNAKNTLYLQMNSLRAEDTAVYYCARSGYYMSDIWGQGTLVTVSS

SEQ ID NO: 346 - protein, 254 aa, synthetic construct
QAVLTQPPSVSVAPGKTARITCGGNNIGSKSVHWYQQKPGQAPVLVVYDDSDRPSGIPERFSGSNSGNTATLTISRVEAGDEADYYCQVWDSSSDHYVFGTGTKVTVLGSRGGGGSGGGGSGGGGSLEMAQVQLVQSGAEVKKPGSSVKVSCKASGDTFSSYAVSWVRQAPGQGLEWMGAINLIFGTVKYAQKFQGRITITADESTSTAYMELSSLRSEDTAVYYCARGHWSQVWWTSHSYDLWGQGTLVTVSS

SEQ ID NO: 347 - protein, 249 aa, synthetic construct
SSELTQDPAVSVALGQTVRITCQGDSLRSYYASWYQQKPGQAPVLVIYDKNNRPSGIPDRFSGSSSGNTASLTITGAQAEDEADYYCNSRDSSGNHHVVFGGGTKLTVLGSRGGGGSGGGGSGGGGSLEMAQVQLVQSGTEVRKPGASLKVSCTTSGYIFTGYYIHWVRQVPGQGLEWMGWINPNSGVTEFAQGFQGRITMTRDTSTSTVYMELSRLTSDDTAVYYCAREGVWGYYDSWGQGTLVTVSS

SEQ ID NO: 348 - protein, 246 aa, synthetic construct
DIQLTQSPSSLSASVGDRVTISCRASQSINKYLNWYQQKPGEAPKLLIYGALRLQSGVPSRFSGSGSGTDYALTITSLQPEDFATYYCQQSYSTPLTFGGGTKVDIKRSRGGGGSGGGGSGGGGSLEMAEVQLVQSGAEVKKPGESLRISCKDSGYSFTSYWISWVRQMPGKGLEWMGRIDPSDSYTNYSPSFQGHVTISADKSISTAYLQWSSLKASDTAMYYCARMEIYSPDYWGQGTLVTVSS

SEQ ID NO: 349 - protein, 4 aa, Homo sapiens
FPNA